(19) United States
(12) Patent Application Publication
(10) Pub. No.: US 2024/0190962 A1
(43) Pub. Date: Jun. 13, 2024

(54) IMMUNOCYTOKINES AND USES THEREOF

(71) Applicant: Immunowake Inc., Birmingham, AL (US)

(72) Inventors: Ellen WU, Birmingham, AL (US); Xiaoyun WU, Birmingham, AL (US); John WAKEFIELD, Birmingham, AL (US)

(21) Appl. No.: 18/269,203

(22) PCT Filed: Dec. 23, 2021
(86) PCT No.: PCT/US2021/073107

§ 371 Date: Jun. 22, 2023

Related U.S. Application Data
(60) Provisional application No. 63/130,339, filed on Dec. 23, 2020.

(51) Int. Cl. C07K 16/28; A61P 35/00; C07K 14/54; C07K 14/55; C07K 14/555

(52) U.S. Cl. CPC C07K 16/2818; A61P 35/00; C07K 14/5434; C07K 14/55; C07K 14/555; C07K 2317/53; C07K 2317/92; C07K 2319/30

(57) ABSTRACT

The present application relates to immunocytokines comprising a cytokine or variant thereof positioned at the hinge region of a heavy chain of an antibody (e.g., full-length antibody), or positioned at a hinge region between an antigen-binding fragment (e.g., ligand, receptor, or antibody fragment) and an Fc domain subunit or portion thereof, methods of making, and uses thereof.

### CROSS REFERENCE TO RELATED APPLICATIONS

This application claims priority benefit of U.S. Provisional Patent Application No. 63/130,339 filed Dec. 23, 2020, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 754392000640SEQLIST.TXT, date recorded: Dec. 23, 2021, size: 1,037,246 bytes).

### FIELD OF THE INVENTION

The present invention relates to immunocytokines comprising a cytokine or variant thereof positioned at the hinge region of a heavy chain of an antibody (e.g., full-length antibody), or positioned at a hinge region between an antigen-binding fragment (e.g., ligand, receptor, or antibody fragment) and an Fc domain subunit or portion thereof, methods of making, and uses thereof.

### BACKGROUND OF THE INVENTION

Cytokines are key regulators of the innate and adaptive immune system that enable immune cells to communicate with each other. Cytokine therapy for activating the immune system of cancer patients continue to be a key area of interest for clinical cancer research. A significant challenge for cytokine monotherapy is to achieve effective anti-tumor responses without causing treatment-limiting toxicities. This dilemma is well exemplified by the low response rates and notorious toxicities of IL-2 and IL-12 therapy. High doses of IL-2 are found to induce vascular leak syndrome (VLS), tumor tolerance caused by activation-induced cell death (AICD), and immunosuppression caused by the activation of regulatory T cells (Tregs). These severe side effects often restrict optimal IL-2 dosing, which limits the number of patients who successfully respond to the therapy. IL-12 has demonstrated modest anti-tumor responses in clinical trials, but often accompanied by significant issues with toxicity (Lasek et al., Cancer Immunollmmunother., 2014). IL-12 treatment was found to associate with systemic flu-like symptoms (e.g., fever, chills, fatigue, erythromelalgia, and headache) and toxic effects on bone marrow and liver. Dosing studies showed that patients could only tolerate IL-12 under 1 μg/kg, far below therapeutically effective dose. Either used as monotherapy or in combination with other agents, IL-12 failed to demonstrate potent sustained therapeutic efficacy in clinical trials (Lasek et al., 2014).

Several approaches have been taken to overcome issues with cytokine monotherapy. Recently, NKTR-214, a recombinant human IL-2 conjugated with polyethylene glycol (PEG; “IL-2-PEG”), has shown promising results in animal models. IL-2-PEG offers two benefits.

First, steric hindrance of PEG masks the region on IL-2 that interacts with IL-2 receptor a (IL-2Rα) subunit responsible for activating immunosuppressive Tregs, biasing activity towards tumor killing CD8+ T cells (Charych et al., Clin Cancer Res., 2016). Second, the conjugation of PEG greatly improves plasma half-life and inproteolytic-stability and decreases immunogenicity and hepatic uptake (Chaffee et al., J Clin Invest., 1992; Pyatak et al., Res Commun Chem Pathol Pharmacol., 1980). Targeted delivery of cytokines (e.g., IL-12) to tumor sites by localized injection or by use of immunocytokines (cytokines fused to antibodies, antibody fragments, or ligand/receptor-Fc fusion protein) have also been developed to overcome side effects of cytokine therapy. Immunocytokines can target cytokines to cells or tissues of interest, such as tumor cells or immune effector cells (Klein et al., Oncoimmunology, 2017; King et al., J Clin Oncol., 2004).

The disclosures of all publications, patents, patent applications and published patent applications referred to herein are hereby incorporated herein by reference in their entirety.

### BRIEF SUMMARY OF THE INVENTION

One aspect of the present application provides an immunocytokine comprising: a) an antigen-binding protein (e.g., antibody, or ligand/receptor-Fc fusion protein) specifically recognizing a target antigen; and b) a cytokine or variant thereof, the antigen-binding protein comprises an antigen-binding polypeptide (e.g., heavy chain, or ligand/receptor-Fc fusion polypeptide) comprising from N′ to C′: an antigen-binding fragment (e.g., antibody fragment, ligand, or receptor), a hinge region, and an Fc domain subunit or portion thereof, and the cytokine or variant thereof is positioned at (e.g., at the N-terminus of, at the C-terminus of, or within) the hinge region.

In some embodiments according to any one of the immunocytokines described above, in the presence of binding of the antigen-binding protein (e.g., antibody, or ligand/receptor-Fc fusion protein) to the target antigen, the activity of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein to the target antigen.

In some embodiments according to any one of the immunocytokines described above, in the absence of binding of the antigen-binding protein (e.g., antibody, or ligand/receptor-Fc fusion protein) to the target antigen, the activity of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state.

In some embodiments according to any one of the immunocytokines described above, the cytokine or variant thereof is a cytokine variant, and the activity of the cytokine variant in a free state is no more than about 80% (such as no more than about any of 70%, 60%, 50%, 40%, 30%, 20%, 10%, or 5%) of that of a corresponding wildtype cytokine in a free state.

In some embodiments according to any one of the immunocytokines described above, the antigen-binding protein (e.g., antibody, or ligand/receptor-Fc fusion protein) comprises two antigen-binding polypeptides (e.g., heavy chain, or ligand/receptor-Fc fusion polypeptide) each comprising a hinge region, and only one antigen-binding polypeptide comprises the cytokine or variant thereof positioned at the hinge region. In some embodiments, the antigen-binding protein comprises two antigen-binding polypeptides each comprising a hinge region, and each antigen-binding polypeptide comprises a cytokine or variant thereof positioned at the hinge region.

In some embodiments according to any one of the immunocytokines described above, the immunocytokine comprises two or more (e.g., 2, 3, 4, or more) cytokines or variants thereof, and the two or more cytokines or variant thereof are positioned in tandem at the hinge region of the antigen-binding polypeptide (e.g., heavy chain, or ligand/receptor-Fc fusion polypeptide).

In some embodiments according to any one of the immunocytokines described above, the cytokine or variant thereof is a monomeric cytokine or variant thereof (e.g., IL-2, IFN-α such as IFN-α2b). In some embodiments, the cytokine or variant thereof is a dimeric cytokine or variant thereof (e.g., IFN-γ, IL-10, IL-12, or IL-23). In some embodiments, both subunits of the dimeric cytokine or variant thereof are positioned in tandem at the hinge region of the antigen-binding polypeptide (e.g., heavy chain, or ligand/receptor-Fc fusion polypeptide). In some embodiments, the antigen-binding protein comprises two antigen-binding polypeptides each comprising a hinge region, and one subunit of the dimeric cytokine or variant thereof is positioned at the hinge region of one antigen-binding polypeptide, and the other subunit of the dimeric cytokine or variant thereof is positioned at the hinge region of the other antigen-binding polypeptide.

In some embodiments according to any one of the immunocytokines described above, the two or more (e.g., 2, 3, 4, or more) cytokines or variants thereof are the same. In some embodiments, the two or more (e.g., 2, 3, 4, or more) cytokines or variants thereof are different.

In some embodiments according to any one of the immunocytokines described above, the antigen-binding protein is a monospecific antigen-binding protein (e.g., monospecific antibody or ligand/receptor-Fc fusion protein). In some embodiments, the antigen-binding protein is a multispecific antigen-binding protein (e.g., multispecific antibody or ligand/receptor-Fc fusion protein).

In some embodiments according to any one of the immunocytokines described above, the target antigen is selected from the group consisting of TIGIT, PD-1, PD-L1, PD-L2, CTLA-4, CD3, CD4, CD8, CD123, CD25, and HER2.

In some embodiments according to any one of the immunocytokines described above, the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, VEGF, erythropoietin, thrombopoietin, G-CSF, M-CSF, SCF, and GM-CSF. In some embodiments, the cytokine or variant thereof is selected from the group consisting of IL-2, IL-10, IL-12, IL-23, IFN-α, and IFN-γ. In some embodiments, the cytokine or variant thereof is IL-2 or variant thereof. In some embodiments, the IL-2 variant comprises one or more mutations at a position selected from the group consisting of L18, Q22, F24, K35, R38, F42, K43, E61, P65, Q126, and S130 relative to a wildtype IL-2 comprising the sequence of SEQ ID NO: 1. In some embodiments, the IL-2 variant comprises one or more mutations selected from the group consisting of L18R, Q22E, F24A, R38D, K43E, E61R, P65L, Q126T, and S130R relative to a wildtype IL-2 comprising the sequence of SEQ ID NO: 1. In some embodiments, the IL-2 variant comprises an R38D/K43E/E61R mutation, an L18R/Q22E/R38D/K43E/E61R mutation, an R38D/K43E/E61R/Q126T mutation, an L18R/Q22E/R38D/K43E/E61R/Q126T mutation, or an L18R/Q22E/R38D/K43E/E61R/Q126T/S130R mutation relative to a wildtype IL-2 comprising the sequence of SEQ ID NO: 1. In some embodiments, the IL-2 variant comprises the sequence of any one of SEQ ID NOs: 2 and 251-254. In some embodiments, the cytokine or variant thereof is IFN-α or variant thereof. In some embodiments, the IFN-α variant comprises one or more mutations at a position selected from the group consisting of R22, L26, F27, L30, K31, D32, R33, H34, D35, F36, S68, T79, K83, Y85, Y89, R120, K121, Y122, Q124, Y129, K131, E132, R144, and E146 relative to an IFN-α comprising the sequence of SEQ ID NO: 3. In some embodiments, the IFN-α variant comprises one or more mutations selected from the group consisting of L30A, K31A, D32A, R33A, H34A, and D35A relative to an IFN-α comprising the sequence of SEQ ID NO: 3. In some embodiments, the IFN-α variant comprises the sequence of any of SEQ ID NOs: 4-9. In some embodiments, the IFN-α variant comprises an L30A mutation relative to an IFN-α comprising the sequence of SEQ ID NO: 3. In some embodiments, the IFN-α variant comprises the sequence of SEQ ID NO: 4. In some embodiments, the cytokine or variant thereof is IFN-γ or variant thereof. In some embodiments, the IFN-γ variant comprises one or more mutations within one or both IFN-γ subunits at a position selected from the group consisting of V5, S20, D21, V22, A23, D24, N25, G26, H111, and Q115 relative to a wildtype IFN-γ subunit comprising the sequence of SEQ ID NO: 10. In some embodiments, the IFN-γ variant comprises one or more mutations within one or both IFN-γ subunits selected from the group consisting of S20A, D21A, D21K, V22A, A23S, A23E, A23Q, A23V, D24A, D24E, N25A, N25K, and H111D relative to a wildtype IFN-γ subunit comprising the sequence of SEQ ID NO: 10. In some embodiments, the IFN-γ variant comprises one or more mutations within one or both IFN-γ subunits selected from the group consisting of S20A/D21A, D21K, V22A/A23S, D24A/N25A, A23E/D24E/N25K, A23Q, and A23V relative to a wildtype IFN-γ subunit comprising the sequence of SEQ ID NO: 10. In some embodiments, one or both subunits of the IFN-γ variant comprises the sequence of any of SEQ ID NOs: 11-17. In some embodiments, the IFN-γ variant comprises an A23V mutation within one or both IFN-γ subunits relative to a wildtype IFN-γ subunit comprising the sequence of SEQ ID NO: 10. In some embodiments, the one or both subunits of the IFN-γ variant comprises the sequence of SEQ ID NO: 13. In some embodiments, the two subunits of the IFN-γ or variant thereof are connected by a linker. In some embodiments, the IFN-γ variant comprises the sequence of SEQ ID NO: 19. In some embodiments, the cytokine or variant thereof is IL-10 or variant thereof. In some embodiments, the IL-10 variant comprises one or more mutations within one or both IL-10 subunits at a position selected from the group consisting of N21, M22, R24, D25, L26, R27, D28, A29, F30, S31, R32, H90, and S93 relative to a wildtype IL-10 subunit comprising the sequence of SEQ ID NO: 20. In some embodiments, the IL-10 variant comprises one or more mutations within one or both IL-10 subunits selected from the group consisting of R24A, D25A, L26A, R27A, D28A, A29S, F30A, S31A, and R32A relative to a wildtype IL-10 subunit comprising the sequence of SEQ ID NO: 20. In some embodiments, the IL-10 variant comprises one or more mutations within one or both IL-10 subunits selected from the group consisting of R24A, D25A/L26A, R27A, D28A/A29S, F30A/S31A, and R32A relative to a wildtype IL-10 subunit comprising the sequence of SEQ ID NO: 20. In some embodiments, one or both subunits of the IL-10 variant comprises the sequence of any of SEQ ID NOs: 21-26. In some embodiments, the IL-10 variant comprises an R27A mutation within one or both IL-10 subunits relative to a wildtype IL-10 subunit comprising the sequence of SEQ ID NO: 20. In some embodiments, the one or both subunits of the IL-10 variant comprises the sequence of SEQ ID NO: 23. In some embodiments, the two subunits of the IL-10 or variant thereof are connected by a linker. In some embodiments, the IL-10 variant comprises the sequence of SEQ ID NO: 28. In some embodiments, the cytokine or variant thereof is IL-12 or variant thereof. In some embodiments, the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30. In some embodiments, the IL-12 variant comprises one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30. In some embodiments, the p40 subunit of the IL-12 variant comprises the sequence of any of SEQ ID NOs: 31-34. In some embodiments, the IL-12 variant comprises an E59A/F60A mutation within the p40 subunit relative to a wildtype p40 subunit comprising the sequence of SEQ ID NO: 30. In some embodiments, the p40 subunit of the IL-12 variant comprises the sequence of SEQ ID NO: 31. In some embodiments, the IL-12 variant comprises an F60A mutation within the p40 subunit relative to a wildtype p40 subunit comprising the sequence of SEQ ID NO: 30. In some embodiments, the p40 subunit of the IL-12 variant comprises the sequence of SEQ ID NO: 33. In some embodiments, the p40 subunit and the p35 subunit of the IL-12 or variant thereof are connected by a linker. In some embodiments, the IL-12 variant comprises the sequence of SEQ ID NO: 36, 275, 331, or 332. In some embodiments, the cytokine or variant thereof is IL-23 or variant thereof. In some embodiments, the IL-23 variant comprises one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30. In some embodiments, the IL-23 variant comprises one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30. In some embodiments, the p40 subunit of the IL-23 variant comprises the sequence of any of SEQ ID NOs: 31-34. In some embodiments, the IL-23 variant comprises an E59A/F60A mutation within the p40 subunit relative to a wildtype p40 subunit comprising the sequence of SEQ ID NO: 30. In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the linker comprises the sequence of any of SEQ ID NOs: 194-242, such as any of SEQ ID NOs: 227-229.

In some embodiments according to any one of the immunocytokines described above, the cytokine or variant thereof is positioned within the hinge region of the antigen-binding polypeptide (e.g., heavy chain, or ligand/receptor-Fc fusion polypeptide).

In some embodiments according to any one of the immunocytokines described above, the Fc domain subunit or portion thereof comprises a knobs-into-holes (KIH) mutation.

In some embodiments according to any one of the immunocytokines described above, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain. In some embodiments, the antibody is an anti-HER2 antibody comprising: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the antibody comprises a VH comprising the sequence of SEQ ID NO: 150 and a VL comprising the sequence of SEQ ID NO: 151. In some embodiments, the antibody comprises a light chain comprising the sequence of SEQ ID NO: 154, (i) the cytokine or variant thereof is an IL-2 variant comprising the sequence of SEQ ID NO: 2, and the heavy chain comprising the IL-2 variant positioned at the hinge region comprises the sequence of SEQ ID NO: 156; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 157; (iii) the cytokine or variant thereof is an IFN-γ variant comprising the sequence of SEQ ID NO: 19, and the heavy chain comprising the IFN-γ variant positioned at the hinge region comprises the sequence of SEQ ID NO: 158; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 159; (v) the cytokine or variant thereof is an IL-12 variant comprising the sequence of SEQ ID NO: 36, and the heavy chain comprising the IL-12 variant positioned at the hinge region comprises the sequence of SEQ ID NO: 160; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 161. In some embodiments, the antibody comprises a second heavy chain not comprising the cytokine or variant thereof positioned at the hinge region, and the second heavy chain comprises the sequence of SEQ ID NO: 155. In some embodiments, the antibody is an anti-CD3 antibody comprising: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the antibody comprises a VH comprising the sequence of SEQ ID NO: 91 and a VL comprising the sequence of SEQ ID NO: 92. In some embodiments, the antibody comprises a light chain comprising the sequence of SEQ ID NO: 94, (i) the cytokine or variant thereof is an IL-2 variant comprising the sequence of SEQ ID NO: 2, and the heavy chain comprising the IL-2 variant positioned at the hinge region comprises the sequence of SEQ ID NO: 96; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 97; (iii) the cytokine or variant thereof is an IFN-γ variant comprising the sequence of SEQ ID NO: 19, and the heavy chain comprising the IFN-γ variant positioned at the hinge region comprises the sequence of SEQ ID NO: 98; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 99; (v) the cytokine or variant thereof is an IL-12 variant comprising the sequence of SEQ ID NO: 36, and the heavy chain comprising the IL-12 variant positioned at the hinge region comprises the sequence of SEQ ID NO: 100; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 101. In some embodiments, the antibody comprises a second heavy chain not comprising the cytokine or variant thereof positioned at the hinge region, and the second heavy chain comprises the sequence of SEQ ID NO: 95. In some embodiments, the antibody is an anti-PD-1 antibody comprising VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the antibody comprises a VH comprising the sequence of SEQ ID NO: 102 and a VL comprising the sequence of SEQ ID NO: 103. In some embodiments, the antibody comprises a light chain comprising the sequence of SEQ ID NO: 106, (i) the cytokine or variant thereof is an IL-2 variant comprising the sequence of SEQ ID NO: 2, and the heavy chain comprising the IL-2 variant positioned at the hinge region comprises the sequence of SEQ ID NO: 108; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 109; (iii) the cytokine or variant thereof is an IFN-γ variant comprising the sequence of SEQ ID NO: 19, and the heavy chain comprising the IFN-γ variant positioned at the hinge region comprises the sequence of SEQ ID NO: 110; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 111; (v) the cytokine or variant thereof is an IL-12 variant comprising the sequence of SEQ ID NO: 36, and the heavy chain comprising the IL-12 variant positioned at the hinge region comprises the sequence of SEQ ID NO: 112; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 113. In some embodiments, the antibody comprises a second heavy chain not comprising the cytokine or variant thereof positioned at the hinge region, and the second heavy chain comprises the sequence of SEQ ID NO: 107. In some embodiments, the antibody is an anti-CD4 antibody comprising: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the antibody comprises a VH comprising the sequence of SEQ ID NO: 73 and a VL comprising the sequence of SEQ ID NO: 74. In some embodiments, the antibody comprises a light chain comprising the sequence of SEQ ID NO: 77, (i) the cytokine or variant thereof is an IL-2 variant comprising the sequence of SEQ ID NO: 2, and the heavy chain comprising the IL-2 variant positioned at the hinge region comprises the sequence of SEQ ID NO: 79; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 80; (iii) the cytokine or variant thereof is an INF-γ variant comprising the sequence of SEQ ID NO: 19, and the heavy chain comprising the IFN-γ variant positioned at the hinge region comprises the sequence of SEQ ID NO: 81; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 82; (v) the cytokine or variant thereof is an IL-12 variant comprising the sequence of SEQ ID NO: 36, and the heavy chain comprising the IL-12 variant positioned at the hinge region comprises the sequence of SEQ ID NO: 83; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 84. In some embodiments, the antibody comprises a second heavy chain not comprising the cytokine or variant thereof positioned at the hinge region, and the second heavy chain comprises the sequence of SEQ ID NO: 78. In some embodiments, the antibody is an anti-CD8 antibody comprising VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the antibody comprises a VH comprising the sequence of SEQ ID NO: 114 and a VL comprising the sequence of SEQ ID NO: 115. In some embodiments, the antibody comprises a light chain comprising the sequence of SEQ ID NO: 117, (i) the cytokine or variant thereof is an IL-2 variant comprising the sequence of SEQ ID NO: 2, and the heavy chain comprising the IL-2 variant positioned at the hinge region comprises the sequence of SEQ ID NO: 119; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 120; (iii) the cytokine or variant thereof is an INF-γ variant comprising the sequence of SEQ ID NO: 19, and the heavy chain comprising the IFN-γ variant positioned at the hinge region comprises the sequence of SEQ ID NO: 121; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 122; (v) the cytokine or variant thereof is an IL-12 variant comprising the sequence of SEQ ID NO: 36, and the heavy chain comprising the IL-12 variant positioned at the hinge region comprises the sequence of SEQ ID NO: 123; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 124. In some embodiments, the antibody comprises a second heavy chain not comprising the cytokine or variant thereof positioned at the hinge region, and the second heavy chain comprises the sequence of SEQ ID NO: 118. In some embodiments, the antibody is an anti-CTLA-4 antibody comprising VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the antibody comprises a VH comprising the sequence of SEQ ID NO: 125 and a VL comprising the sequence of SEQ ID NO: 126. In some embodiments, the antibody comprises a light chain comprising the sequence of SEQ ID NO: 129, (i) the cytokine or variant thereof is an IL-2 variant comprising the sequence of SEQ ID NO: 2, and the heavy chain comprising the IL-2 variant positioned at the hinge region comprises the sequence of SEQ ID NO: 131; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 132; (iii) the cytokine or variant thereof is an INF-γ variant comprising the sequence of SEQ ID NO: 19, and the heavy chain comprising the IFN-γ variant positioned at the hinge region comprises the sequence of SEQ ID NO: 133; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 134; (v) the cytokine or variant thereof is an IL-12 variant comprising the sequence of SEQ ID NO: 36, and the heavy chain comprising the IL-12 variant positioned at the hinge region comprises the sequence of SEQ ID NO: 135; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 136. In some embodiments, the antibody comprises a second heavy chain not comprising the cytokine or variant thereof positioned at the hinge region, and the second heavy chain comprises the sequence of SEQ ID NO: 130. In some embodiments, the antibody is an anti-PD-L1 antibody comprising: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the antibody comprises a VH comprising the sequence of SEQ ID NO: 137 and a VL comprising the sequence of SEQ ID NO: 138. In some embodiments, the antibody comprises a light chain comprising the sequence of SEQ ID NO: 140, (i) the cytokine or variant thereof is an IL-2 variant comprising the sequence of SEQ ID NO: 2, and the heavy chain comprising the IL-2 variant positioned at the hinge region comprises the sequence of SEQ ID NO: 144; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 145; (iii) the cytokine or variant thereof is an INF-γ variant comprising the sequence of SEQ ID NO: 19, and the heavy chain comprising the IFN-γ variant positioned at the hinge region comprises the sequence of SEQ ID NO: 146; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 147; (v) the cytokine or variant thereof is an IL-12 variant comprising the sequence of SEQ ID NO: 36, and the heavy chain comprising the IL-12 variant positioned at the hinge region comprises the sequence of SEQ ID NO: 148; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 149. In some embodiments, the antibody comprises a second heavy chain not comprising the cytokine or variant thereof positioned at the hinge region, and the second heavy chain comprises the sequence of SEQ ID NO: 143. In some embodiments, the antibody is an anti-CD25 antibody comprising VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the antibody comprises a VH comprising the sequence of SEQ ID NO: 162 and a VL comprising the sequence of SEQ ID NO: 163. In some embodiments, the antibody comprises a light chain comprising the sequence of SEQ ID NO: 165, (i) the cytokine or variant thereof is an IL-2 variant comprising the sequence of SEQ ID NO: 2, and the heavy chain comprising the IL-2 variant positioned at the hinge region comprises the sequence of SEQ ID NO: 169; (ii) the cytokine or variant thereof is an IFN-α variant comprising the sequence of SEQ ID NO: 4, and the heavy chain comprising the IFN-α variant positioned at the hinge region comprises the sequence of SEQ ID NO: 170; (iii) the cytokine or variant thereof is an INF-γ variant comprising the sequence of SEQ ID NO: 19, and the heavy chain comprising the IFN-γ variant positioned at the hinge region comprises the sequence of SEQ ID NO: 171; (iv) the cytokine or variant thereof is an IL-10 variant comprising the sequence of SEQ ID NO: 28, and the heavy chain comprising the IL-10 variant positioned at the hinge region comprises the sequence of SEQ ID NO: 172; (v) the cytokine or variant thereof is an IL-12 variant comprising the sequence of SEQ ID NO: 36, and the heavy chain comprising the IL-12 variant positioned at the hinge region comprises the sequence of SEQ ID NO: 173; or (vi) the cytokine or variant thereof is an IL-23 variant comprising the sequence of SEQ ID NO: 39, and the heavy chain comprising the IL-23 variant positioned at the hinge region comprises the sequence of SEQ ID NO: 174. In some embodiments, the antibody comprises a second heavy chain not comprising the cytokine or variant thereof positioned at the hinge region, and the second heavy chain comprises the sequence of SEQ ID NO: 168. In some embodiments, the antibody is and anti-PD-1 antibody with reduced (reduced at least about 2, 3, 4, 5, 6, 7, 8, 9, 10, 20, 50, 100, 1000, or more fold) binding affinity to PD-1 compared to nivolumab. In some embodiments, the antibody is an anti-PD-1 antibody comprising VH-CDR1 and VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103, wherein the VH-CDR3 comprises any one of following mutations relative to SEQ ID NO: 102: D100N, D100G, D100R, N99G, N99A, or N99M. In some embodiments, the antibody comprises a light chain comprising the sequence of SEQ ID NO: 106, wherein the cytokine or variant thereof is an IL-12 variant comprising the sequence of SEQ ID NO: 36, and wherein the heavy chain comprising the IL-12 variant positioned at the hinge region comprises the sequence of any one of SEQ ID NOs: 268-273.

In some embodiments according to any one of the immunocytokines described above, (i) the antigen-binding fragment is a ligand, and the target antigen is a receptor specifically recognized by the ligand; or (ii) the antigen-binding fragment is a receptor, and the target antigen is a ligand specifically recognized by the receptor. In some embodiments, the ligand or receptor is selected from the group consisting of IL-2, IL-2Rα (CD25), PD-1, PD-L1, PD-L2, NKG2A, NKG2C, NKG2F, NKG2D, BCMA, APRIL, BAFF, IL-3, IL-13, LLT1, AICL, DNAM-1, and NKp80. In some embodiments, the ligand is PD-L2. In some embodiments, the ligand comprises the sequence of SEQ ID NO: 176. In some embodiments, (i) the cytokine or variant thereof is an IL-2 variant comprising the sequence of SEQ ID NO: 2, and the antigen-binding polypeptide comprising the IL-2 variant positioned at the hinge region comprises the sequence of SEQ ID NO: 180; (ii) the cytokine or variant thereof is an IFN-α variant comprising the sequence of SEQ ID NO: 4, and the antigen-binding polypeptide comprising the IFN-α variant positioned at the hinge region comprises the sequence of SEQ ID NO: 182; (iii) the cytokine or variant thereof is an INF-γ variant comprising the sequence of SEQ ID NO: 19, and the antigen-binding polypeptide comprising the IFN-γ variant positioned at the hinge region comprises the sequence of SEQ ID NO: 183; (iv) the cytokine or variant thereof is an IL-10 variant comprising the sequence of SEQ ID NO: 28, and the antigen-binding polypeptide comprising the IL-10 variant positioned at the hinge region comprises the sequence of SEQ ID NO: 184; (v) the cytokine or variant thereof is an IL-12 variant comprising the sequence of SEQ ID NO: 36, and the antigen-binding polypeptide comprising the IL-12 variant positioned at the hinge region comprises the sequence of SEQ ID NO: 186; (vi) wherein the cytokine or variant thereof is an IL-12 variant comprising the sequence of SEQ ID NO: 275, and wherein the antigen-binding polypeptide comprising the IL-12 variant positioned at the hinge region comprises the sequence of SEQ ID NO: 274; or (vii) the cytokine or variant thereof is an IL-23 variant comprising the sequence of SEQ ID NO: 39, and the antigen-binding polypeptide comprising the IL-23 variant positioned at the hinge region comprises the sequence of SEQ ID NO: 187. In some embodiments, the antigen-binding protein comprises a second antigen-binding polypeptide not comprising the cytokine or variant thereof positioned at the hinge region, and the second antigen-binding polypeptide comprises the sequence of any of SEQ ID NOs: 178, 179, 181, and 185. In some embodiments, the ligand is or derives from extracellular domain of PD-L1. In some embodiments, the PD-L1 ligand has increased (e.g., increased at least about any of 2, 3, 4, 5, 6, 10, 20, 50, 100, 100, or more fold) binding to PD-1 compared to wild-type PD-L1. In some embodiments, the PD-L1 ligand comprises any mutations or combinations thereof, relative to SEQ ID NO: 249: 154Q, E58M, R113T, Mi 15L, S117A, and G119K. In some embodiments, the PD-L1 ligand comprises any of below mutations relative to SEQ ID NO: 249: E58M/R113T/M115L/S117A/G119K, I54Q/E58M/R113T/M115L/S117A/G119K, I54Q/R113T/M115L/S117A/G119K, I54Q/E58M/M115L/S117A/G119K, I54Q/E58M/R113T/S117A/G119K, I54Q/E58M/R113T/M115L/G119K, or I54Q/E58M/R113T/M115L/S117A. In some embodiments, the PD-L1 ligand comprises the sequence of any one of SEQ ID NOs: 255-261. In some embodiments, the cytokine or variant thereof is an IL-12 variant comprising the sequence of SEQ ID NO: 36, and the antigen-binding polypeptide comprising the IL-12 variant positioned at the hinge region comprises the sequence of SEQ ID NO: 277. In some embodiments, the antigen-binding protein comprises a second antigen-binding polypeptide comprising a second cytokine or variant thereof positioned at the hinge region or at the C′ of the Fc fragment. In some embodiments, the second cytokine or variant is any cytokine or variant described herein, such as IL-2 or IFN-γ or variant thereof. In some embodiments, the antigen-binding protein comprises a second antigen-binding polypeptide comprising a second cytokine or variant thereof positioned at the hinge region, and the second antigen-binding polypeptide comprises the sequence of any of SEQ ID NOs: 285-288 and 327. In some embodiments, the ligand is or derives from extracellular domain of PD-L2. In some embodiments, the PD-L2 ligand has increased (e.g., increased at least about any of 2, 3, 4, 5, 6, 10, 20, 50, 100, 100, or more fold) binding to PD-1 compared to wild-type PD-L2. In some embodiments, the PD-L2 ligand comprises any mutations or combinations thereof, relative to SEQ ID NO: 176: T56V, S58V, Q60L, or T56V/S58V/Q60L. In some embodiments, the PD-L2 ligand comprises the sequence of any one of SEQ ID NOs: 262-265. In some embodiments, the cytokine or variant thereof is an IL-12 variant comprising the sequence of SEQ ID NO: 36, and wherein the antigen-binding polypeptide comprising the IL-12 variant positioned at the hinge region comprises the sequence of SEQ ID NO: 293. In some embodiments, the antigen-binding protein comprises a second antigen-binding polypeptide comprising a second cytokine or variant thereof positioned at the hinge region or C′ of Fc fragment. In some embodiments, the second cytokine or variant is any cytokine or variant described herein, such as IL-2 or IFN-γ or variant thereof. In some embodiments, the ligand is or derives from extracellular domain of CD155. In some embodiments, the CD155 ligand comprises the sequence of SEQ ID NO: 267.

The present application also provides any anti-PD-1 antibodies described herein, such as anti-PD-1 antibody with HC-CDR3 mutations described herein.

The present application also provides any immunocytokines described herein, such as any constructs described in the Examples, including single chain fusion of cytokine at the hinge region, double chain fusion of cytokein (same or different) at the hinge region, or one cytokine fused at hinge region of one chain and the other fused to C′ of Fc of the other chain. In some embodiments, the cytokine is any of the cytokein or variant described herein, such as IL-2, IL-12, IFN-γ etc.

Also provided herein are immunocytokines comprising cytokine or variant thereof fused to the C′ of an antigen-binding protein specifically recognizing a target antigen (e.g., at C′ of Fc fragment). In some embodiments, the immunocytokine comprises: a) an antigen-binding protein specifically recognizing a target antigen; and b) a cytokine or variant thereof (e.g., any described herein), wherein the antigen-binding protein comprises an antigen-binding polypeptide comprising from N′ to C′: an antigen-binding fragment (e.g., anti-PD-1, PD-L1, or PD-L2), a hinge region, and an Fc domain subunit or portion thereof, and wherein the cytokine or variant thereof is positioned at the C′ of the Fc domain subunit or portion thereof.

Also provided herein are immunocytokines comprising a) an antigen-binding protein specifically recognizing a target antigen; b) a first cytokine (e.g., IL-2 or IFN-γ) or variant thereof, and c) a second cytokine (e.g., IL-12) or variant thereof wherein the antigen-binding protein comprises a first antigen-binding polypeptide comprising from N′ to C′: an antigen-binding fragment, a hinge region, and an Fc domain subunit or portion thereof, and wherein the first cytokine or variant thereof is positioned at the hinge region; and a second antigen-binding polypeptide comprising from N′ to C′: an antigen-binding fragment, a hinge region, and an Fc domain subunit or portion thereof, and wherein the second cytokine or variant thereof is positioned at the hinge region.

Also provided herein are immunocytokines comprising a) an antigen-binding protein specifically recognizing a target antigen; b) a first cytokine (e.g., IL-2 of IFN-γ) or variant thereof, and c) a second cytokine (e.g., IL-12) or variant thereof wherein the antigen-binding protein comprises a first antigen-binding polypeptide comprising from N′ to C′: an antigen-binding fragment, a hinge region, and an Fc domain subunit or portion thereof, and wherein the first cytokine or variant thereof is positioned at the hinge region; and a second antigen-binding polypeptide comprising from N′ to C′: an antigen-binding fragment, a hinge region, and an Fc domain subunit or portion thereof, and wherein the second cytokine or variant thereof is positioned at the C′ of Fc domain.

Another aspect of the present application provides a method of selectively activating the activity of a cytokine or variant thereof to a cell expressing a target antigen in an individual, comprising administering to the individual an effective amount of an immunocytokine, the immunocytokine comprises: a) an antigen-binding protein (e.g., antibody, or ligand/receptor-Fc fusion protein) specifically recognizing the target antigen; and b) a cytokine or variant thereof, the antigen-binding protein comprises an antigen-binding polypeptide (e.g., heavy chain, or ligand/receptor-Fc fusion polypeptide) comprising from N′ to C′: an antigen-binding fragment (e.g., antibody fragment, ligand, or receptor), a hinge region, and an Fc domain subunit or portion thereof, and the cytokine or variant thereof is positioned at (e.g., at the N-terminus of, at the C-terminus of, or within) the hinge region, and the activity of the cytokine or variant thereof is selectively activated upon binding of the antigen-binding protein to the target antigen. In some embodiments, the present application provides a method of selectively activating the activity of a cytokine or variant thereof to a cell expressing a target antigen in an individual, comprising administering to the individual an effective amount of any one of the immunocytokines described above, and the activity of the cytokine or variant thereof is selectively activated upon binding of the antigen-binding protein to the target antigen. In some embodiments, in the presence of binding of the antigen-binding protein to the target antigen, the activity of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein to the target antigen. In some embodiments, in the absence of binding of the antigen-binding protein to the target antigen, the activity of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant, and the activity of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain. In some embodiments, (i) the antigen-binding fragment is a ligand, and the target antigen is a receptor specifically recognized by the ligand; or (ii) the antigen-binding fragment is a receptor, and the target antigen is a ligand specifically recognized by the receptor.

Further provided are isolated nucleic acids encoding any one of the immunocytokines described herein, vectors (e.g., lentiviral vector) comprising such nucleic acids, host cells (e.g., CHO cell) comprising such nucleic acids or vectors, and methods of producing any one of the immunocytokines described herein.

Also provided are compositions (e.g., pharmaceutical compositions), kits, and articles of manufacture comprising any of the immunocytokines described herein. Methods of treating a disease or disorder (e.g., cancer, infection, autoimmune disease, allergy, graft rejection, or graft-versus-host disease (GvHD)) in an individual using an effective amount of any of the immunocytokines or compositions (e.g., pharmaceutical compositions) described herein are also provided.

### BRIEF DESCRIPTION OF THE DRAWINGS

FIG. 1A depicts full-length antibody structure. FIG. 1B depicts an exemplary immunocytokine structure comprising a cytokine or variant thereof fused to the N-terminus of a subunit of the Fc fragment of a parental full-length antibody. FIG. 1C depicts an exemplary immunocytokine structure comprising a cytokine or variant thereof fused to the C-terminus of a heavy chain of a parental full-length antibody. FIG. 1D depicts an exemplary immunocytokine structure comprising a cytokine or variant thereof fused to the N-terminus of a heavy chain variable domain (VH) of a parental full-length antibody. FIG. 1E depicts an exemplary immunocytokine structure comprising a cytokine or variant thereof fused to the C-terminus of a light chain constant region (CL) of a parental full-length antibody. Filled light grey portions indicate parental antibody backbone.

FIGS. 2A-2D depict exemplary immunocytokine structures of the present invention, in which one or more cytokines or variants thereof (or subunits thereof) are positioned at the hinge region of one or both heavy chains of a parental full-length antibody. FIG. 2A depicts a monomeric cytokine or variant thereof (e.g., IL-2, or IFN-α) positioned at the hinge region of one heavy chain of a parental full-length antibody. FIG. 2B depicts a dimeric (homodimeric or heterodimeric) cytokine or variant thereof (e.g., IFN-γ, IL-10, IL-12, or IL-23) expressed in a single chain and positioned at the hinge region of one heavy chain of a parental full-length antibody. FIG. 2C depicts two cytokines or variants thereof positioned in tandem at the hinge region of one heavy chain of a parental full-length antibody. FIG. 2D depicts two cytokines or variants thereof each positioned at the hinge region of one heavy chain of a parental full-length antibody, or a dimeric (homodimeric or heterodimeric) cytokine or variant thereof with each subunit positioned at the hinge region of one heavy chain of a parental full-length antibody. Filled light grey portions indicate parental antibody backbone.

FIGS. 3A-3C depict alternative exemplary immunocytokine structures of the present invention, in which a cytokine or variant thereof is positioned between a VH (e.g., within a Fab or an scFv) and a subunit of an Fc fragment. Light grey portions indicate parental antibody backbone.

FIGS. 4A-4C depict exemplary immunocytokine structures of the present invention, in which one or more cytokines or variants thereof (or subunits thereof) are positioned at the hinge region of one or both polypeptides of a parental ligand/receptor-hinge-Fc fusion protein. FIG. 4A depicts a monomeric cytokine or variant thereof (e.g., IL-2, or IFN-α) positioned at the hinge region of one polypeptide of a parental ligand/receptor-hinge-Fc fusion protein. FIG. 4B depicts two cytokines or variants thereof each positioned at the hinge region of one polypeptide of a parental ligand/receptor-hinge-Fc fusion protein, or a dimeric (homodimeric or heterodimeric) cytokine or variant thereof with each subunit positioned at the hinge region of one polypeptide of a parental ligand/receptor-hinge-Fc fusion protein. FIG. 4C depicts a dimeric (homodimeric or heterodimeric) cytokine or variant thereof (e.g., IFN-γ, IL-10, IL-12, or IL-23) expressed in a single chain, or two cytokines or variants thereof fused in tandem, positioned at the hinge region of one polypeptide of a parental ligand/receptor-hinge-Fc fusion protein. Light grey portions indicate parental ligand/receptor-hinge-Fc fusion protein backbone.

FIG. 5 depicts tumor volume in 4T1 syngeneic tumor mice treated with IL-12(E59A/F60A)/anti-PD-1 immunocytokine (#48), IL-12(E59A/F60A)/PD-L2-Fc immunocytokine (#29), IL-12(E59A/F60A)/IL-2(R38D/K43E/E61R)/anti-PD-1 immunocytokine (#54), or PBS (negative control). Black arrows indicate injection days.

FIG. 6 depicts tumor volume in EMT6 syngeneic tumor mice treated with IL-12(E59A/F60A)/anti-PD-1 immunocytokine (#48), IL-12(E59A/F60A)/PD-L2-Fc immunocytokine (#29), IL-2(R38D/K43E/E61R)/PD-L2-Fc immunocytokine (#11), or PBS (negative control). Black arrows indicate injection days.

FIGS. 7A and 7B depict tumor volume in CT26 syngeneic tumor mice treated with IL-12(F60A)/PD-L2-Fc immunocytokine (#30), PD-L2-Fc/IL-12(F60A) immunocytokine (#34), or PBS (negative control). Black arrows indicate injection days. FIG. 7A shows the average tumor volume for all mice receiving the indicated IL-12 immunocytokine or control. The average tumor size (±STD) of each group when the first treatment was administered is shown in parenthesis. FIG. 7B shows tumor volumes for individual mice receiving the indicated IL-12 immunocytokine.

FIG. 8 depicts tumor volumes in mice previously treated with IL-12(F60A)/PD-L2-Fc immunocytokine (#30) or PD-L2-Fc/IL-12(F60A) immunocytokine (#34) against CT26 tumor, then re-challenged with CT26 murine colon cancer cells on the right flank and EMT6 murine breast cancer cells on the left flank (as control).

FIG. 9 depicts tumor volume in late-stage CT26 syngeneic tumor mice treated with IL-12(E59A/F60A)/PD-L2 Fc immunocytokine (#29) or IL-12(F60A)/PD-L2-Fc immunocytokine (#30). Black arrows indicate injection days. The tumor size of each mouse when the first treatment was administered is shown in parenthesis.

FIG. 10 shows the regression of tumor size in a late-stage CT26 syngeneic tumor mouse model treated with IL-12(F60A)/PD-L2-Fc immunocytokine (construct #30).

FIGS. 11A and 11B depict tumor volume in EMT6 syngeneic breast tumor mice treated with IL-12(E59A/F60A)/PD-L2 Fc immunocytokine (#29), IL-12(F60A)/PD-L2-Fc immunocytokine (#30), IL-12(E59A/F60A)/anti-PD-1 immunocytokine (#48), or PBS (negative control). Black arrows indicate injection days. FIG. 11A shows the average tumor volume of all mouse groups, with the average tumor size (±STD) when the first treatment was administered shown in parenthesis. FIG. 11B shows tumor volumes for individual mouse receiving the indicated IL-12 immunocytokines.

FIG. 12 depicts tumor volumes in mice previously treated with IL-12(E59A/F60A)/PD-L2 Fc immunocytokine (#29), IL-12(F60A)/PD-L2-Fc immunocytokine (#30), or IL-12(E59A/F60A)/anti-PD-1 immunocytokine (#48) against EMT6 tumor, then re-challenged with EMT6 murine breast cancer cells on the right flank and CT26 murine colon cancer cells on the left flank (as control).

FIG. 13 shows 4T1 murine breast cancer tumors extracted from mammary gland fat pad of mice treated with IL-12(E59A/F60A)/PD-L2-Fc immunocytokine (#29), IL-12(F60A)/PD-L2-Fc (#30), a combination of anti-PD-1 and anti-CTLA-4 antibodies, or PBS (negative control).

FIG. 14 depicts 4T1 murine breast cancer cells metastasized to lungs in mice injected with 4T1 cells at mammary gland fat pad and treated with IL-12(E59A/F60A)/PD-L2-Fc immunocytokine (#29), IL-12(F60A)/PD-L2-Fc (#30), a combination of anti-PD-1 and anti-CTLA-4 antibodies, or PBS (negative control).

FIGS. 15A and 15B depict tumor volume in B16 melanoma syngeneic tumor mice treated with IL-12(F60A)/PD-L2-Fc immunocytokine (#30), PD-L2-Fc/IL-12(F60A) immunocytokine (#34), or PBS (negative control). Black arrows indicate injection days. FIG. 15A shows the average tumor volume of all mouse groups, with the average tumor size (±STD) when the first treatment was administered shown in parenthesis. FIG. 15B shows tumor volumes for individual mouse receiving the indicated IL-12 immunocytokines.

FIGS. 16A and 16B depict tumor volume in LL2 lung cancer syngeneic mice treated with IL-12(F60A)/PD-L2-Fc immunocytokine (#30), PD-L2-Fc/IL-12(F60A) immunocytokine (#34), or PBS (negative control). Black arrows indicate injection days. FIG. 16A shows the average tumor volume of all mouse groups, with the average tumor size (±STD) when the first treatment was administered shown in parenthesis. FIG. 16B shows tumor volumes for individual mouse receiving the indicated IL-12 immunocytokines.

### DETAILED DESCRIPTION OF THE INVENTION

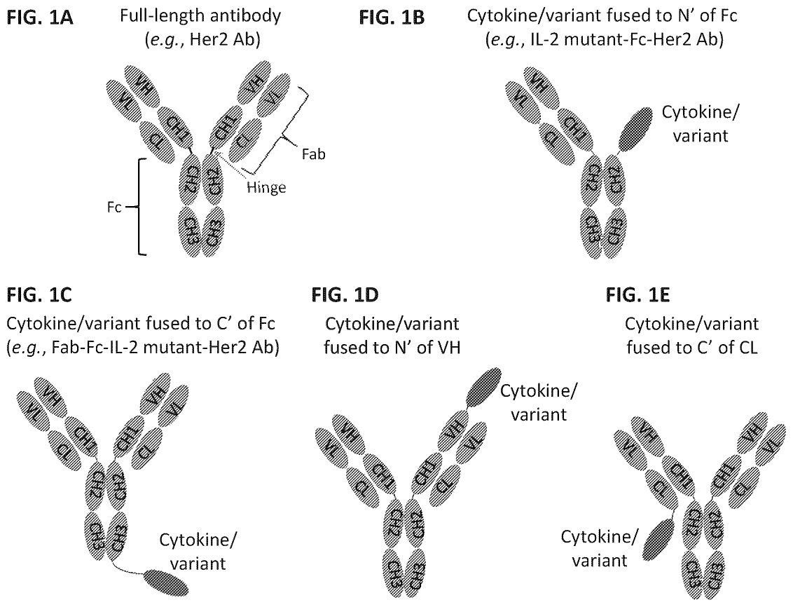

Cytokines are key mediators of innate and adaptive immunity. However, cytokine therapy (e.g., for treating cancer) have shown limited success due to severe toxicity, which limits the dosing far below therapeutically effective dose. Immunocytokines, which are constructs with cytokines fused to antibodies, antigen-binding fragments, ligand-Fc fusion protein, or receptor-Fc fusion protein (hereinafter collectively referred to as “ligand/receptor-Fc fusion protein” or “ligand/receptor-hinge-Fc fusion protein”) can deliver cytokines to target cells (e.g., tumor cells, or immune effector cells) or tissues with the recognition of target antigens by the antibodies or antigen-binding fragments (e.g., antibody fragments, ligands, or receptors) within immunocytokines, which can both reduce non-specific (off-target) cytokine activities and/or associated toxicities (e.g., toxicities on healthy cells or tissues), and concentrate cytokine therapeutic effects at target sites (e.g., disease sites). The activation of immunocytokines can occur via trans-activation, which requires specific binding of the antibody or antigen-binding fragment to target antigens on tumor cells; or cis-activation, which requires specific binding of the antibody or antigen-binding fragment to target antigens on immune cells. Most immunocytokines developed nowadays have the cytokine moiety fused to the N-terminus or the C-terminus of the heavy chain or the light chain of a full-length antibody (such as Hul4.8-IL2, NHS-IL2LT, NHS-IL12, BC1-IL12; see, e.g., FIGS. 1C-1E) or fused to the N-terminus or the C-terminus of an antigen-binding fragment (e.g., diabody, scFv, such as L19-IL2 or F16-IL2), so cytokine-receptor binding/activation can still occur even in the absence of antibody-antigen recognition, leading to off-target toxicities.

The present invention provides immunocytokines with unique configurations that address the issues faced by current cytokine/immunocytokine therapy. Particularly, immunocytokines of the present invention decrease non-specific activities (i.e., antibody or antigen-binding fragment-independent binding) and increase specific activities (i.e., antibody or antigen-binding fragment-dependent binding) of cytokines by positioning the cytokine moiety (e.g., cytokine or variant thereof) at a hinge region in between an antigen-binding fragment (e.g., ligand, receptor, VHH, scFv, or Fab) and an Fc domain subunit or portion thereof (e.g., CH2-CH3 fragment, or CH2 only, or CH3 only), for example, at a hinge region in between an scFv and an Fc domain subunit (e.g., an antigen-binding polypeptide comprising VH-VL-cytokine-Fc subunit, or VL-VH-cytokine-Fc subunit), at a hinge region in between the Fab and the Fc domain of a full-length antibody (e.g., an antigen-binding polypeptide comprising VH-CH1-cytokine-Fc subunit), or at a hinge region in between a ligand (or a receptor) and an Fc domain subunit (e.g., an antigen-binding polypeptide comprising ligand-cytokine-Fc subunit, or receptor-cytokine-Fc subunit). Without being bound by theory, it is believed that steric hindrance of the antigen-binding fragment (e.g., ligand, receptor, VHH, scFv, Fab) and the Fc domain or portion thereof reduces accessibility of the cytokine or variant thereof to its receptor, or “masks” the cytokine or variant thereof from binding to its receptor, in the absence of target antigen binding by the antigen-binding fragment. Upon binding of the target antigen, on the other hand, the cytokine becomes activated. Surprisingly, unlike other immunocytokine designs which “expose” the cytokine moiety at its N-terminus or C-terminus, the unique immunocytokine configuration of the present invention requires binding of the antigen-binding fragment (e.g., ligand, receptor, VHH, scFv, or Fab) to its target antigen first before binding of the cytokine moiety to its receptor can occur, thus ensuring that cytokine receptor activation is entirely target antigen-binding dependent (on-target). With this enhanced targeting specificity design, cytokines can be safely delivered to target sites (e.g., tumor cells, or immune cells) to achieve therapeutic effects.

Further, Inventors of the application discovered that certain immunocytokines, such as constructed with a parental antigen-binding protein (e.g., antibody or fragment thereof, or receptor- or ligand-Fc fusion protein) and a cytokine moiety with opposing effects in regulating immune responses, demonstrated significantly better toxicity profile and therapeutic efficacy. For example, when positioning an IL-12 cytokine (pro-inflammatory) at the hinge region of a PD-L2 extracellular domain-hinge-Fc fusion protein, the resulting IL-12/PD-L2-Fc immunocytokine not only specifically targeted IL-12 activity (e.g., activity of binding to IL-12 receptor, and/or IL-12 pro-inflammatory activity) to PD-1+ target cells, but also stimulated PD-1 inhibitory immune checkpoint signaling via PD-L2-PD-1 binding, thus creating an immunosuppression signal that “balances” against the immunostimulating activity of IL-12 (hereinafter also referred to as “balancing immunocytokine”). Any agonist antibodies or ligands (e.g., PD-L2, PD-L1, CD80, or CD86) that can activate or stimulate an immunosuppressive signaling pathway (e.g., by binding to an inhibitory immune checkpoint molecule such as PD-1 or CTLA-4), or any antagonist antibodies, ligands, or receptors that can reduce or block an immunostimulatory signaling pathway (e.g., e.g., by binding to a stimulatory immune checkpoint molecule such as CD27 or CD28 or an immunostimulatory receptor such as IL-2R) can be used in combination with an immunostimulating cytokine or variant thereof (e.g., IL-2, IL-12, or IL-23) to construct a balancing immunocytokine with any of the immunocytokine configurations described herein. Any antagonist antibodies, ligands, or receptors that can reduce or block an immunosuppressive signaling pathway (e.g., by binding to an inhibitory immune checkpoint molecule such as PD-1 or CTLA-4), or any agonist antibodies or ligands (e.g., CD70, CD80, CD86, or IL-2) that can activate or stimulate an immunostimulatory signaling pathway (e.g., e.g., by binding to a stimulatory immune checkpoint molecule such as CD27 or CD28 or an immunostimulatory receptor such as IL-2R) can be used in combination with an immunosuppressive cytokine or variant thereof (e.g., IL-10, IL-27, IL-35, TGF-β) to construct a balancing immunocytokine with any of the immunocytokine configurations described herein. Such “balancing” of immune response design adds an additional regulatory layer to fine-tune the bioactivity and toxicity of immunocytokines described herein, in addition to their unique targeting specificity design.

The immunocytokines described herein can also treat various advanced and/or hard-to-treat cancer types (e.g., TNBC, melanoma, lung cancer), inhibiting cancer metastasis, treating or delaying tumor progression of cancer types that are resistant to current immunotherapies (e.g., anti-PD-1 therapy, anti-CTLA-4 therapy, or a combination therapy thereof), and/or extending life-span of such patients. The immunocytokines described herein have superior safety profiles with significantly reduced side effects comprared to other immunocytokine formats, such as C′ Fc fusion.

In combination with this unique immunocytokine structure design, various cytokine variants have also been generated and positioned at the hinge region to screen for cytokine variants with little or no activity (e.g., activity of binding to cytokine receptor, and/or cytokine biological activity) in the absence of target antigen-binding, but increased activity “rescued” or “revealed” in the presence of target antigen-binding. Various immunocytokines have been obtained that can specifically target cytokine (e.g., IFN-α2b, IL-12, IL-23, IL-10, or IFN-γ) activity towards target cells (e.g., PD-1+ cells, CD4+ T cells, or CD8+ T cells), with minimal or no cytokine activity towards non-target cells (e.g., PD-1− cells, CD8+ T cells, or CD4+ T cells).

Accordingly, one aspect of the present application provides an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., cell surface antigen, receptor, or ligand); and b) a cytokine or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region.

In some embodiments, the parental antigen-binding protein (i.e., the antigen-binding protein backbone used for constructing the immunocytokine described herein) is an antibody (e.g., full-length antibody) or fragment thereof specifically recognizing the target antigen. In some embodiments, the antigen-binding polypeptide comprising the hinge region is a heavy chain of the antibody, and wherein the cytokine or variant thereof is positioned at the hinge region of the heavy chain. Thus, in some embodiments, the present invention provides an immunocytokine comprising: a) an antibody (e.g., a full-length antibody) specifically recognizing a target antigen; and b) a cytokine or variant thereof, wherein the antibody comprises a heavy chain comprising a hinge region, and wherein the cytokine or variant thereof is positioned at (e.g., at the N′ of, at the C′ of, or within) the hinge region. The immunocytokine described herein in some embodiments comprises: (a) a first antigen-binding polypeptide comprising from N-terminus to C-terminus: a first VH domain, an optional first CH1, a cytokine moiety at a hinge region, and a first subunit of an Fc domain or portion thereof (e.g., CH2+CH3, or CH2 only); (b) a second antigen-binding polypeptide comprising from N-terminus to C-terminus: a second VH domain, an optional second CH1, a hinge region, and a second subunit of the Fc domain or portion thereof (e.g., CH2+CH3, or CH2 only); (c) a third antigen-binding polypeptide comprising from N-terminus to C-terminus: a first VL domain and an optional first CL; and (d) a fourth antigen-binding polypeptide comprising from N-terminus to C-terminus: a second VL domain and an optional second CL; wherein the first VH and the first VL and optionally the first CH1 and first CL form a first antigen-binding domain (e.g., Fab) specifically recognizing a first target antigen; and wherein the second VH and the second VL and optionally the second CH1 and second CL form a second antigen-binding domain (e.g., Fab) specifically recognizing a second target antigen. In some embodiments, the antigen-binding polypeptide comprising the hinge region is an scFv-hinge-Fc fusion polypeptide. The immunocytokine described herein in some embodiments comprises: (a) a first antigen-binding polypeptide comprising from N-terminus to C-terminus: (a first VH domain and a first VL domain) or (a first VL domain and a first VH domain), a cytokine moiety at a hinge region, and a first subunit of an Fc domain or portion thereof (e.g., CH2+CH3, or CH2 only); and (b) a second antigen-binding polypeptide comprising from N-terminus to C-terminus: (a second VH domain and a second VL domain) or (a second VL domain and a second VH domain), a hinge region, and a second subunit of the Fc domain or portion thereof (e.g., CH2+CH3, or CH2 only); wherein the first VH and the first VL form a first antigen-binding domain (e.g., scFv) specifically recognizing a first target antigen; and wherein the second VH and the second VL and form a second antigen-binding domain (e.g., scFv) specifically recognizing a second target antigen. In some embodiments, the antigen-binding polypeptide comprising the hinge region is a VHH-hinge-Fc fusion polypeptide. The immunocytokine described herein in some embodiments comprises: (a) a first antigen-binding polypeptide comprising from N-terminus to C-terminus: a first VHH, a cytokine moiety at a hinge region, and a first subunit of an Fc domain or portion thereof (e.g., CH2+CH3, or CH2 only); and (b) a second antigen-binding polypeptide comprising from N-terminus to C-terminus: a second VHH, a hinge region, and a second subunit of the Fc domain or portion thereof (e.g., CH2+CH3, or CH2 only); wherein the first VHH specifically recognizes a first target antigen; and wherein the second VHH specifically recognizes a second target antigen.

In some embodiments, the parental antigen-binding protein is an antigen-binding fragment-hinge-Fc fusion protein. In some embodiments, the antigen-binding fragment is an scFv. In some embodiments, the antigen-binding fragment is a VHH. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. Thus, in some embodiments, the present invention provides an immunocytokine comprising: a) an antigen-binding fragment-hinge-Fc fusion protein specifically recognizing a target antigen (e.g., cell surface antigen, receptor, or ligand); and b) a cytokine or variant thereof, wherein the antigen-binding fragment-hinge-Fc fusion protein comprises an antigen-binding fragment-hinge-Fc fusion polypeptide comprising from N′ to C′: an antigen-binding fragment (e.g., ligand, receptor, VHH, or scFv), a hinge region, and an Fc domain subunit or portion thereof, and wherein the cytokine or variant thereof is positioned at (e.g., at the N′ of, at the C′ of, or within) the hinge region. The immunocytokine described herein in some embodiments comprises: (a) a first antigen-binding polypeptide comprising from N-terminus to C-terminus: a first antigen-binding fragment (e.g., ligand, receptor, VHH, or scFv), a cytokine moiety at a hinge region, and a first subunit of an Fc domain or portion thereof (e.g., CH2+CH3, or CH2 only); and (b) a second antigen-binding polypeptide comprising from N-terminus to C-terminus: a second antigen-binding fragment (e.g., ligand, receptor, VHH, or scFv), a hinge region, and a second subunit of the Fc domain or portion thereof (e.g., CH2+CH3, or CH2 only); wherein the first antigen-binding fragment specifically recognizes a first target antigen (e.g., cell surface antigen, receptor, or ligand); and wherein the second antigen-binding fragment specifically recognizes a second target antigen (e.g., cell surface antigen, receptor, or ligand).

Also provided are isolated nucleic acids encoding such immunocytokines, vectors comprising such nucleic acids, host cells comprising such nucleic acids or vectors, methods of producing such immunocytokines, pharmaceutical compositions and articles of manufacture comprising such immunocytokines, methods of selectively activating the activity of a cytokine or variant thereof to a cell expressing a target antigen with such immunocytokines or pharmaceutical compositions thereof, and methods of treating diseases (e.g., cancer, viral infection, autoimmune diseases) with such immunocytokines or pharmaceutical compositions thereof.

### I. Definitions

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

The term “immunocytokine”, as used herein refers to an antigen-binding protein (e.g., antibody, or antigen-binding fragment (e.g., ligand, receptor, or antibody fragment)) format, which is fused to a cytokine molecule. The antigen-binding protein (e.g., antibody, or antigen-binding fragment (e.g., ligand, receptor, or antibody fragment)) format may be any of those described herein, and the cytokine may be fused directly, or by means of a linker or chemical conjugation to the antigen-binding protein format.

The term “cytokine storm,” also known as a “cytokine cascade” or “hypercytokinemia,” is a potentially fatal immune reaction typically consisting of a positive feedback loop between cytokines and immune cells, with highly elevated levels of various cytokines (e.g., INF-γ, IL-10, IL-6, CCL2, etc.).

As used herein, when an antigen-binding protein (e.g., antibody, antigen-binding fragment, or ligand) is referred to as an “antagonist” of a target antigen (e.g., a receptor, or an immune checkpoint molecule), it means that upon target antigen binding, the antigen-binding protein (e.g., antibody, antigen-binding fragment, or ligand) blocks, suppresses, or reduces the biological activity of the target antigen (e.g., blocks receptor signaling). For example, an anti-PD-1 antagonist antibody is an antibody that reduces or blocks PD-1 signaling. When an antigen-binding protein (e.g., antibody, antigen-binding fragment, or ligand) is referred to as an “agonist” of a target antigen (e.g., a receptor, or an immune checkpoint molecule), it means that upon target antigen binding, the antigen-binding protein (e.g., antibody, antigen-binding fragment, or ligand) stimulates, activates, or enhances the biological activity of the target antigen (e.g., activates receptor signaling). For example, a wildtype PD-L2 ligand (e.g., extracellular domain) is an agonist that activates PD-1 signaling.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment. For example, an individual is successfully “treated” if one or more symptoms associated with viral infection are mitigated or eliminated, including, but are not limited to, reducing the proliferation of (or destroying) infectious virus, decreasing symptoms resulting from the disease (e.g., cytokine storm), increasing the quality of life of those suffering from the disease, decreasing the dose of other medications required to treat the disease, and/or prolonging survival of individuals.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease (e.g., cancer) progression that may be initially undetectable and includes occurrence, recurrence, and onset.

The term “effective amount” used herein refers to an amount of an agent or a combination of agents, sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with the cancer; (viii) stimulate or activate immune cells (e.g., immune effector cells), e.g. for immune response, such as to produce cytokine(s), or for immune cell proliferation and/or differentiation; and/or (ix) prevent, reduce, or eliminate inflammation or autoimmune response, such as inhibiting pro-inflammatory cytokine secretion. In the case of viral infection, the effective amount of the agent may inhibit (i.e., reduce to some extent and preferably abolish) virus activity; control and/or attenuate and/or inhibit inflammation or a cytokine storm induced by said viral pathogen; prevent worsening, arrest and/or ameliorate at least one symptom of said viral infection or damage to said subject or an organ or tissue of said subject, emanating from or associated with said viral infection; control, reduce, and/or inhibit cell necrosis in infected and/or non-infected tissue and/or organ; control, ameliorate, and/or prevent the infiltration of inflammatory cells (e.g., NK cells, cytotoxic T cells, neutrophils) in infected or non-infected tissues and/or organs; and/or stimulate or activate immune cells (e.g., immune effector cells), e.g., for immune response, such as to produce cytokine(s), or for immune cell proliferation and/or differentiation.

As used herein, an “individual” or a “subject” refers to a mammal, including, but not limited to, human, bovine, horse, feline, canine, rodent, or primate. In some embodiments, the individual is a human.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody” includes conventional 4-chain antibodies, single-domain antibodies, and antigen-binding fragments thereof.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for p and E isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see e.g., Basic and ClinicalImmunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

An “isolated” antibody (or construct) is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody (or construct) includes the antibody in situ within recombinant cells since at least one component of the antibody's natural environment will not be present. Ordinarily, however, an isolated polypeptide, antibody, or construct will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH”. VHH is thus a special type of V_{H}. The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the heavy chain and light chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts.

Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: MonoclonalAntibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

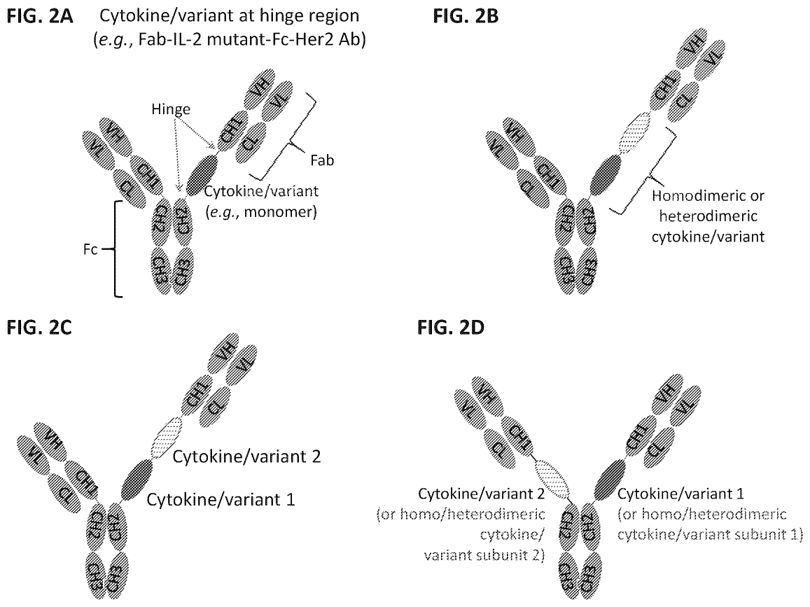

The terms “full-length antibody”, “intact antibody”, or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain variable domain (such as VHH) and an Fc region. The constant domains may be native sequence constant domains (e.g., human native sequence constant domains) or amino acid sequence variants thereof. In some cases, the intact antibody may have one or more effector functions. It is to be understood that for the present invention, reference to a “full-length antibody” also includes a full-length antibody backbone or parental full-length antibody (e.g., full-length 4-chain antibody, or full-length heavy-chain only antibody) whose hinge region has a cytokine moiety positioned therein (see, e.g., FIGS. 2A-2D).

An “antibody fragment”, “antigen-binding domain”, or “antigen-binding fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody (scFv) molecules; single-domain antibodies (such as VHH), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy-terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. It is to be understood that for the present invention, reference to an “antigen-binding domain” or “antigen-binding fragment” also includes a ligand that can specifically recognizes a target receptor, or a receptor that can specifically recognizes a target ligand.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or CL) domain of the light chain.

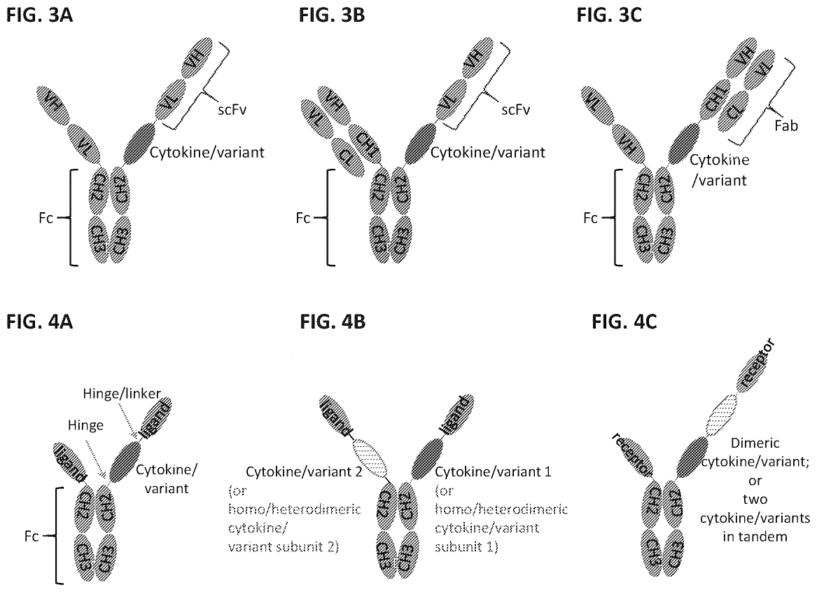

The “heavy chain” of antibodies (immunoglobulins) can be divided into three functional regions: the Fd region, the hinge region, and the Fc region (fragment crystallizable). The Fd region comprises the VH and CH1 domains and, in combination with the light chain, forms Fab—the antigen-binding fragment. The Fc fragment is responsible for the immunoglobulin effector functions, which include, for example, complement fixation and binding to cognate Fc receptors of effector cells. The hinge region, found in IgG, IgA, and IgD immunoglobulin classes, acts as a flexible spacer that allows the Fab portion to move freely in space relative to the Fc region. In contrast to the constant regions, the hinge domains are structurally diverse, varying in both sequence and length among immunoglobulin classes and subclasses. For heavy-chain only antibody, “heavy chain” includes the heavy chain variable domain (such as VHH), a hinge region, and an Fc region. It is to be understood that for the present invention, reference to a “heavy chain” also includes a heavy chain comprising a VH domain, a hinge region, and an Fc domain or portion thereof (e.g., VL-VH-hinge-Fc domain subunit, or VH-VL-hinge-Fc domain subunit), and a heavy chain (e.g., heavy chain of a full-length 4-chain antibody, an VH-hinge-Fc-containing antibody, or heavy chain of a heavy-chain only antibody) comprising a cytokine moiety positioned at the hinge region (see, e.g., FIGS. 2A-3C).

The “light chains” of antibodies (immunoglobulins) from any mammalian species can be assigned to one of two clearly distinct types, called kappa (“x”) and lambda (“X”), based on the amino acid sequences of their constant domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site. “Single-chain Fv” also abbreviated as “sFv” or “scFv” are antibody fragments that comprise the V_{H} and V_{L} antibody domains connected into a single polypeptide chain. Preferably, the scFv polypeptide further comprises a polypeptide linker between the V_{H} and V_{L} domains which enables the scFv to form the desired structure for antigen binding. For a review of the scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natd. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). “Humanized antibody” is used as a subset of “chimeric antibodies”.

“Humanized” forms of non-human (e.g., llama or camelid) antibodies are chimeric antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991).

A number of HVR delineations are in use and are encompassed herein. The Kabat Complementarity Determining Regions (CDRs) are based on sequence variabilitγ and α re the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table A.

TABLE A
HVR delineations

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the VH, the subgroup may be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “affinity-matured” antibody is one with one or more alterations in one or more CDRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by V_{H}- and V_{L}-domain shuffling. Random mutagenesis of CDR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “epitope” means a protein determinant capable of specific binding to an antibody or antigen-binding fragment (e.g., ligand, receptor, VHH, scFv, Fab, etc.). Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. As used herein, the term “specifically binds”, “specifically recognizes”, or is “specific for” refers to measurable and reproducible interactions such as binding between a target and an antigen-binding protein (or cytokine and cytokine receptor), which is determinative of the presence of the target (or cytokine) in the presence of a heterogeneous population of molecules including biological molecules. For example, an antigen binding protein (such as a Fab) that specifically binds a target (which can be an epitope) is an antigen binding protein that binds this target with greater affinity, avidity, more readily, and/or with greater duration than it binds other targets. A cytokine that specifically binds a cytokine receptor is a cytokine that binds this cytokine receptor with greater affinity, avidity, more readily, and/or with greater duration than it binds other cytokine receptors. In some embodiments, the extent of binding of an antigen binding protein (or cytokine) to an unrelated target (or unrelated cytokine receptor) is less than about 10% of the binding of the antigen binding protein (or cytokine) to the target as measured (or the cytokine receptor as measured), e.g., by a radioimmunoassay (RIA). In some embodiments, an antigen binding protein that specifically binds a target (or a cytokine that specifically binds a cytokine receptor) has a dissociation constant (K_{D}) of ≤10⁻⁵ M, ≤10⁻⁶ M, ≤10⁻⁷ M, ≤10⁻¹ M, ≤10⁻⁹ M, ≤10¹⁰ M, ≤10⁻¹ M, or ≤10⁻¹² M. In some embodiments, an antigen binding protein (or cytokine receptor) specifically binds an epitope on a protein (or cytokine) that is conserved among the protein from different species. In some embodiments, specific binding can include, but does not require exclusive binding. Binding specificity of the antigen-binding protein (or cytokine and cytokine receptor) can be determined experimentally by any protein binding methods known in the art. Such methods comprise, but are not limited to Western blots, ELISA-, RIA-, ECL-, IRMA-, EIA-, BIACORE™-tests and peptide scans.

The term “specificity” refers to selective recognition of an antigen binding protein for a particular epitope of an antigen. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. Unless otherwise indicated, the order in which the antigens bound by a bispecific antibody listed is arbitrary. That is, for example, the terms “anti-CD3/HIER2,” “anti-HER2/CD3,” “CD3 xHER2” and “HER2xCD3” may be used interchangeably to refer to bispecific antibodies that specifically bind to both CD3 and HER2. The term “monospecific” as used herein denotes an antigen binding protein that has one or more binding sites each of which bind the same epitope of the same antigen.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

“Antibody effector functions” refer to those biological activities attributable to the Fc region (a native sequence Fc region or amino acid sequence variant Fc region) of an antibody and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectorless mutation.” In some embodiments, the effectorless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased Clq binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

The term “Fc region,” “fragment crystallizable region,” “Fc fragment,” or “Fc domain” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody or Fc-fusion protein, or by recombinantly engineering the nucleic acid encoding a heavy chain of the antibody or Fc-fusion protein. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the immunocytokines described herein include human IgG1, IgG2 (IgG2A, IgG2B), IgG3 and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fc receptor” or “FcR” describes a receptor that binds the Fc region of an antibody or Fc-fusion protein. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Binding affinity” generally refers to the strength of the sum total of non-covalent interactions between a single binding site of a molecule (e.g., an antibody, antigen-binding fragment (such as ligand, receptor, VHH, scFv, etc.), or cytokine) and its binding partner (e.g., an antigen (such as cell surface molecule, receptor, ligand, etc.), or cytokine receptor). Unless indicated otherwise, as used herein, “binding affinity” refers to intrinsic binding affinity that reflects a 1:1 interaction between members of a binding pair. Binding affinity can be indicated by K_{d}, K_{off}, Kₒₙ, or Kₐ. The term “K_{off}”, as used herein, is intended to refer to the off-rate constant for dissociation of an antibody (or antigen-binding fragment) from the antibody (or antigen-binding fragment)/antigen complex (e.g., ligand-receptor complex), or the off rate constant for dissociation of a cytokine from the cytokine/cytokine receptor complex, as determined from a kinetic selection set up, expressed in units of s⁻¹. The term “Kₒₙ”, as used herein, is intended to refer to the on-rate constant for association of an antibody (or antigen-binding fragment) to the antigen to form the antibody (or antigen-binding fragment)/antigen complex, or the on rate constant for association of a cytokine to the cytokine receptor to form the cytokine/cytokine receptor complex, expressed in units of M⁻¹s⁻¹. The term equilibrium dissociation constant “K_{D}” or “K_{d}”, as used herein, refers to the dissociation constant of a particular antibody (or antigen-binding fragment)-antigen interaction (or cytokine-cytokine receptor interaction), and describes the concentration of antigen (or cytokine) required to occupy one half of all of the antibody-binding domains (or antigen-binding fragment) present in a solution of antibody (or antigen-binding fragment) molecules (or cytokine receptor) at equilibrium, and is equal to K_{off}/Kₒₙ, expressed in units of M. The measurement of Kₐ presupposes that all binding agents are in solution. In the case where the antibody (or antigen-binding fragment) is tethered to a cell wall, e.g., in a yeast expression system, the corresponding equilibrium rate constant is expressed as EC50, which gives a good approximation of Kₐ. The affinity constant, K_{d}, is the inverse of the dissociation constant, K_{d}, expressed in units of M⁻¹. The dissociation constant (K_{D} or K_{d}) is used as an indicator showing affinity of antibodies (or antigen-binding fragments) to antigens (or cytokines to cytokine receptors). For example, easy analysis is possible by the Scatchard method using antibodies (or antigen-binding fragments) marked with a variety of marker agents, as well as by using BIACORE™ X (made by Amersham Biosciences), which is an over-the-counter, measuring kit, or similar kit, according to the user's manual and experiment operation method attached with the kit. The K_{D} value that can be derived using these methods is expressed in units of M (Mols). An antibody or antigen-binding fragment thereof (or cytokine) that specifically binds to a target (or cytokine receptor) may have a dissociation constant (K_{d}) of, for example, ≤10⁻⁵ M, ≤10⁻⁶ M, ≤10⁻⁷ M, ≤10⁻¹ M, ≤10⁻⁹ M, ≤10⁻¹⁰ M, ≤10⁻¹¹ M, or ≤10⁻² M.

Half maximal inhibitory concentration (IC₅₀) is a measure of the effectiveness of a substance (such as an antibody or antigen-binding fragment) in inhibiting a specific biological or biochemical function. It indicates how much of a particular drug or other substance (inhibitor, such as an antibody or antigen-binding fragment) is needed to inhibit a given biological process by half. The values are typically expressed as molar concentration. IC₅₀ is comparable to an “EC₅₀” for agonist drug or other substance (such as an antibody, antigen-binding fragment, or a cytokine). EC₅₀ also represents the plasma concentration required for obtaining 50% of a maximum effect in vivo. As used herein, an “IC₅₀” is used to indicate the effective concentration of an antibody or antigen-binding fragment needed to neutralize 50% of the antigen bioactivity in vitro. IC₅₀ or EC₅₀ can be measured by bioassays such as inhibition of ligand binding by FACS analysis (competition binding assay), cell-based cytokine release assay, or amplified luminescent proximity homogeneous assay (AlphaLISA).

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments (or cytokine subunits) by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the constructs, polypeptides, and antibodies described herein is in a form other than in the form or setting in which it is found in nature. Isolated nucleic acid molecules therefore are distinguished from nucleic acid encoding the constructs, polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Immunocytokines

The present invention in one aspect provides an immunocytokine comprising a cytokine or variant thereof positioned at a hinge region of a heavy chain of an antibody, or positioned at a hinge region between an antigen-binding fragment (e.g., ligand, receptor, or antibody fragment) and an Fc domain subunit or portion thereof. The parental antibody that serves as the backbone for constructing the immunocytokines described herein can be of any antibody or antigen-binding fragment format that comprises a heavy chain comprising a hinge region, such as a full-length 4-chain antibody, a heavy chain only antibody, or an antigen-binding fragment (e.g., scFv, Fab) fused to an Fc domain subunit or portion thereof (e.g., CH2+CH3, or CH2 only) via a hinge region. The parental antigen-binding protein that serves as the backbone for constructing the immunocytokines described herein can also be an antigen-binding fragment-hinge-Fc fusion protein, such as an antigen-binding fragment-hinge-Fc fusion protein comprising two antigen-binding fragment-hinge-Fc fusion polypeptides each comprising from N′ to C′: an antigen-binding fragment (e.g., ligand, or receptor), a hinge region, and an Fc domain subunit or portion thereof.

In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and the cytokine or variant thereof is positioned at the hinge region of the heavy chain. Thus, in some embodiments, there is provided an immunocytokine comprising: a) an antibody (e.g., full-length antibody, heavy chain only antibody, or antigen-binding fragment (e.g., scFv, Fab) fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, between the C-terminus of CH1 and the N-terminus of the hinge region of a heavy chain of the full-length antibody, or between the C-terminus of the antigen-binding fragment and the N-terminus of the hinge region). In some embodiments, there is provided an immunocytokine comprising: a) an antibody specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antibody comprises a heavy chain, and wherein the heavy chain comprises from N-terminus to C-terminus: a VH domain, optionally a CH1 domain, the cytokine or variant thereof at a hinge region, a CH2 domain, and optionally a CH3 domain. In some embodiments, there is provided an immunocytokine comprising: (a) a first antigen-binding polypeptide comprising from N-terminus to C-terminus: a first VH domain, an optional first CH1, a cytokine (e.g., IL-2, IFN-α (e.g., IFN-α2b), IFN-γ, IL-10, IL-12, or IL-23) or variant thereof at a first hinge region (e.g., the cytokine moiety is between the C-terminus of CH1 and the N-terminus of the hinge region, or between the C-terminus of the hinge region and the N-terminus of the Fc domain subunit, or within the hinge region), and a first subunit of an Fc domain or portion thereof (e.g., CH2+CH3, or CH2 only); (b) a second antigen-binding polypeptide comprising from N-terminus to C-terminus: a second VH domain, an optional second CH1, a second hinge region, and a second subunit of the Fc domain or portion thereof (e.g., CH2+CH3, or CH2 only); (c) a third antigen-binding polypeptide comprising from N-terminus to C-terminus: a first VL domain and an optional first CL; and (d) a fourth antigen-binding polypeptide comprising from N-terminus to C-terminus: a second VL domain and an optional second CL; wherein the first VH and the first VL and optionally the first CH1 and first CL form a first antigen-binding domain (e.g., Fab) specifically recognizing a first target antigen (e.g., CTLA-4, PD-L1, PD-L2, CD25, HER2, PD-1, CD3, CD4, CD123, or CD8); and wherein the second VH and the second VL and optionally the second CH1 and second CL form a second antigen-binding domain (e.g., Fab) specifically recognizing a second target antigen (e.g., CTLA-4, PD-L1, PD-L2, CD25, HER2, PD-1, CD3, CD4, CD123, or CD8). In some embodiments, the immunocytokine described herein comprises a full-length parental antibody (hereinafter also referred to as “full-length antibody immunocytokine”). Thus in some embodiments, there is provided an immunocytokine comprising: a) a full-length antibody specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, the antigen-binding protein is an antigen-binding fragment-hinge-Fc fusion protein (the corresponding immunocytokine hereinafter is also referred to as “Fc-fusion protein immunocytokine”), such as scFv-hinge-Fc fusion protein, VHH-hinge-Fc fusion protein, ligand-hinge-Fc fusion protein, or receptor-hinge-Fc fusion protein. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, the parental antigen-binding fragment-hinge-Fc fusion protein used as backbone for constructing the immunocytokine described herein comprises two antigen-binding fragment-hinge-Fc fusion polypeptides each comprising from N′ to C′: an antigen-binding fragment (e.g., ligand, receptor, VHH, or scFv), a hinge region, and an Fc domain subunit or portion thereof. Thus, in some embodiments, there is provided an immunocytokine comprising: a) an antigen-binding fragment-hinge-Fc fusion protein specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding fragment-hinge-Fc fusion protein comprises an antigen-binding fragment-hinge-Fc fusion polypeptide comprising from N′ to C′: an antigen-binding fragment (e.g., ligand, receptor, VHH, or scFv), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided an immunocytokine comprising: (a) a first antigen-binding polypeptide comprising from N-terminus to C-terminus: a first antigen-binding fragment (e.g., ligand, receptor, VHH, or scFv), a cytokine (e.g., IL-2, IFN-α (e.g., IFN-α2b), IFN-γ, IL-10, IL-12, or IL-23) or variant thereof at a first hinge region (e.g., the cytokine moiety is between the C-terminus of the antigen-binding fragment and the N-terminus of the hinge region, between the C-terminus of the hinge region and the N-terminus of the Fc domain subunit, or within the hinge region), and a first subunit of an Fc domain or portion thereof (e.g., CH2+CH3, or CH2 only); (b) a second antigen-binding polypeptide comprising from N-terminus to C-terminus: a second antigen-binding fragment (e.g., ligand, receptor, VHH, or scFv), a second hinge region, and a second subunit of the Fc domain or portion thereof (e.g., CH2+CH3, or CH2 only); wherein the first antigen-binding fragment specifically recognizes a first target antigen (e.g., CTLA-4, PD-L1, PD-L2, CD25, HER2, PD-1, CD3, CD4, CD123, or CD8); and wherein the second antigen-binding fragment specifically recognizes a second target antigen (e.g., CTLA-4, PD-L1, PD-L2, CD25, HER2, PD-1, CD3, CD4, CD123, or CD8). In some embodiments, in the presence of binding of the antigen-binding protein (e.g., antibody (e.g., full-length antibody), or ligand/receptor-hinge-Fc fusion protein) or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen. In some embodiments, in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the antibody (e.g., full-length antibody, or antigen binding fragment fused to Fc domain or portion thereof) comprises two heavy chains each comprising a hinge region, and only one heavy chain comprises the cytokine or variant thereof positioned at the hinge region. In some embodiments, the antigen-binding protein (e.g., antibody such as full-length antibody, or antigen-binding fragment-hinge-Fc fusion protein such as ligand/receptor-hinge-Fc fusion protein) comprises two antigen-binding polypeptides (e.g., antibody heavy chain, or antigen-binding fragment-hinge-Fc fusion polypeptide such as ligand/receptor-hinge-Fc fusion polypeptide) each comprising a hinge region, and wherein only one antigen-binding polypeptide comprises the cytokine or variant thereof positioned at (e.g., at the N′ of, at the C′ of, or within) the hinge region. In some embodiments, the antigen-binding protein (e.g., antibody such as full-length antibody, or antigen-binding fragment-hinge-Fc fusion protein such as ligand/receptor-hinge-Fc fusion protein) comprises two antigen-binding polypeptides (e.g., antibody heavy chain, or antigen-binding fragment-hinge-Fc fusion polypeptide such as ligand/receptor-hinge-Fc fusion polypeptide) each comprising a hinge region, and wherein each antigen-binding polypeptide comprises a cytokine or variant thereof positioned at (e.g., at the N′ of, at the C′ of, or within) the hinge region. In some embodiments, the antibody (e.g., full-length antibody, or antigen binding fragment fused to Fc domain or portion thereof) comprises two heavy chains each comprising a hinge region, and each heavy chain comprises a cytokine or variant thereof positioned at the hinge region. In some embodiments, the immunocytokine comprises two or more (e.g., 2, 3, 4, 5, or more) cytokines or variants thereof, wherein the two or more cytokines or variant thereof are positioned in tandem (e.g., connected via an optional peptide linker) at the hinge region of the antigen-binding polypeptide (e.g., heavy chain of an antibody, or antigen-binding fragment-hinge-Fc fusion polypeptide such as ligand/receptor-hinge-Fc fusion polypeptide). In some embodiments, the cytokine or variant thereof is a monomeric cytokine or variant thereof, such as IL-2 or IFN-α (e.g., IFN-α2a, IFN-α2b, IFN-α2c). In some embodiments, the cytokine or variant thereof is a dimeric cytokine or variant thereof. In some embodiments, the cytokine or variant thereof is a homodimeric cytokine or variant thereof, such as IL-10 or IFN-γ. In some embodiments, the cytokine or variant thereof is a heterodimeric cytokine or variant thereof, such as IL-12 or IL-23.

In some embodiments, both subunits of the dimeric cytokine or variant thereof are positioned in tandem (e.g., connected via an optional peptide linker) at the hinge region of the antigen-binding polypeptide (e.g., heavy chain of an antibody, or antigen-binding fragment-hinge-Fc fusion polypeptide such as ligand/receptor-hinge-Fc fusion polypeptide). In some embodiments, the antigen-binding protein (e.g., antibody such as full-length antibody, or antigen-binding fragment-hinge-Fc fusion protein such as ligand/receptor-hinge-Fc fusion protein) comprises two antigen-binding polypeptides (e.g., heavy chains, or antigen-binding fragment-hinge-Fc fusion polypeptides such as ligand/receptor-hinge-Fc fusion polypeptides) each comprising a hinge region, one subunit of the dimeric cytokine or variant thereof is positioned at the hinge region of one antigen-binding polypeptide (e.g., heavy chain, or antigen-binding fragment-hinge-Fc fusion polypeptide such as ligand/receptor-hinge-Fc fusion polypeptide), and the other subunit of the dimeric cytokine or variant thereof is positioned at the hinge region of the other antigen-binding polypeptide (e.g., heavy chain, or antigen-binding fragment-hinge-Fc fusion polypeptide such as ligand/receptor-hinge-Fc fusion polypeptide). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein (e.g., antibody such as full-length antibody, or antigen-binding fragment-hinge-Fc fusion protein such as ligand/receptor-hinge-Fc fusion protein) is heterodimeric. In some embodiments, the two or more cytokines or variants thereof are the same. In some embodiments, the two or more cytokines or variants thereof are different. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein (e.g., antibody or fragment thereof, or antigen-binding fragment-hinge-Fc fusion protein such as ligand/receptor-hinge-Fc fusion protein) is multispecific. In some embodiments, the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, erythropoietin, VEGF, thrombopoietin, G-CSF, M-CSF, SCF, and GM-CSF. In some embodiments, the cytokine or variant thereof is positioned between the C-terminus of antigen-binding fragment (e.g., scFv, VHH, ligand, receptor) and the N-terminus of the hinge region of the antigen-binding polypeptide. In some embodiments, the heavy chain of the antibody (e.g., full-length antibody) comprises a constant region 1 (CH1), and the cytokine or variant thereof is positioned between the C-terminus of CH1 and the N-terminus of the hinge region of the heavy chain of the antibody. In some embodiments, the cytokine or variant thereof is positioned between the C-terminus of the hinge region and the N-terminus of the Fc domain subunit (or portion thereof) of the antigen-binding polypeptide (e.g., heavy chain of an antibody, or antigen-binding fragment-hinge-Fc fusion protein). In some embodiments, the cytokine or variant thereof is positioned within the hinge region (e.g., replaces an internal portion of the hinge region, inserted within the hinge region and introducing one or more additional hinge or linker amino acids, or inserted within the hinge region without deleting hinge region amino acid) of the antigen-binding polypeptide (e.g., heavy chain of an antibody, or fragment thereof, or antigen-binding fragment-hinge-Fc fusion protein such as ligand/receptor-hinge-Fc fusion protein). In some embodiments, the antigen-binding polypeptide (e.g., heavy chain of an antibody or antigen-binding fragment-hinge-Fc fusion protein such as ligand/receptor-hinge-Fc fusion protein), such as the Fc domain subunit or portion thereof, comprises a knobs-into-holes (KIH) mutation (e.g., each antigen-binding polypeptide (e.g., heavy chain of a full-length antibody) comprises a KIH mutation). In some embodiments, the antigen-binding protein is an agonist antibody (or antigen-binding fragment thereof) or ligand (e.g., PD-L2, PD-L1, CD80, or CD86) that can activate or stimulate an immunosuppressive signaling pathway (e.g., by binding to an inhibitory immune checkpoint molecule such as PD-1 or CTLA-4 on a target cell), and the cytokine or variant thereof is an immunostimulating cytokine or variant thereof (e.g., IL-2, IL-12, or IL-23). In some embodiments, the antigen-binding protein is an antagonist antibody (or antigen-binding fragment thereof), a ligands, or a receptor that can reduce or block an immunostimulatory signaling pathway (e.g., by binding to a stimulatory immune checkpoint molecule such as CD27 or CD28 or an immunostimulatory receptor such as IL-2R), and the cytokine or variant thereof is an immunostimulating cytokine or variant thereof (e.g., IL-2, IL-12, or IL-23). In some embodiments, the antigen-binding protein is an antagonist antibody (or antigen-binding fragment thereof), a ligand, or a receptor that can reduce or block an immunosuppressive signaling pathway (e.g., by binding to an inhibitory immune checkpoint molecule such as PD-1 or CTLA-4), and the cytokine or variant thereof is an immunosuppressive cytokine or variant thereof (e.g., IL-10, IL-27, IL-35, TGF-β). In some embodiments, the antigen-binding protein is an agonist antibody (or antigen-binding fragment thereof) or ligand (e.g., CD70, CD80, CD86, or IL-2) that can activate or stimulate an immunostimulatory signaling pathway (e.g., by binding to a stimulatory immune checkpoint molecule such as CD27 or CD28, or an immunostimulatory receptor such as IL-2R), and the cytokine or variant thereof is an immunosuppressive cytokine or variant thereof (e.g., IL-10, IL-27, IL-35, TGF-β).

In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the cytokine or variant thereof is positioned at (e.g., at the N′ of, at the C′ of, or within) the hinge region; and wherein in the presence of binding of the antigen-binding protein (e.g., antibody (e.g., full-length antibody), or ligand/receptor-hinge-Fc fusion protein) or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen. In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the cytokine or variant thereof is positioned at (e.g., at the N′ of, at the C′ of, or within) the hinge region; and wherein in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the cytokine or variant thereof is positioned at (e.g., at the N′ of, at the C′ of, or within) the hinge region; wherein in the presence of binding of the antigen-binding protein (e.g., antibody (e.g., full-length antibody), or ligand/receptor-hinge-Fc fusion protein) or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen; and wherein in the absence of binding of the antigen-binding protein (e.g., antibody (e.g., full-length antibody), or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, wherein the cytokine or variant thereof is positioned at the hinge region (e.g., within the hinge region, between the C-terminus of CH1 and the N-terminus of the hinge region of a heavy chain of the full-length antibody, or between the C-terminus of the antigen-binding fragment and the N-terminus of the hinge region); and wherein in the presence of binding of the antibody (or antigen binding fragment) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen binding fragment) to the target antigen. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, wherein the cytokine or variant thereof is positioned at the hinge region (e.g., within the hinge region, between the C-terminus of CH1 and the N-terminus of the hinge region of a heavy chain of the full-length antibody, or between the C-terminus of the antigen-binding fragment and the N-terminus of the hinge region); and in the absence of binding of the antibody (or antigen binding fragment) to the target antigen, the activity (binding affinity to corresponding cytokine receptor or subunit thereof, and/or biological activity) of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, wherein the cytokine or variant thereof is positioned at the hinge region (e.g., within the hinge region, between the C-terminus of CH1 and the N-terminus of the hinge region of a heavy chain of the full-length antibody, or between the C-terminus of the antigen-binding fragment and the N-terminus of the hinge region); wherein in the presence of binding of the antibody (or antigen binding fragment) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen binding fragment) to the target antigen; and in the absence of binding of the antibody (or antigen binding fragment) to the target antigen, the activity (binding affinity to corresponding cytokine receptor or subunit thereof, and/or biological activity) of the cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, there is provided an immunocytokine comprising: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; and wherein in the presence of binding of the full-length antibody to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the full-length antibody to the target antigen. In some embodiments, there is provided an immunocytokine comprising: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; and wherein in the absence of binding of the full-length antibody to the target antigen, the activity (binding affinity to corresponding cytokine receptor or subunit thereof, and/or biological activity) of the cytokine or variant thereof positioned at the hinge region of the heavy chain of the full-length antibody is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, there is provided an immunocytokine comprising: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; wherein in the presence of binding of the full-length antibody to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the full-length antibody to the target antigen; and wherein in the absence of binding of the full-length antibody to the target antigen, the activity (binding affinity to corresponding cytokine receptor or subunit thereof, and/or biological activity) of the cytokine or variant thereof positioned at the hinge region of the heavy chain of the full-length antibody is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist.

In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the antigen-binding protein comprises two antigen-binding polypeptides (e.g., antibody heavy chain, or antigen-binding fragment-hinge-Fc fusion polypeptide such as ligand/receptor-hinge-Fc fusion polypeptide) each comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the first cytokine or variant thereof is positioned at (e.g., at the N′ of, at the C′ of, or within) the hinge region of the first antigen-binding polypeptide, and wherein the second cytokine or variant thereof is positioned at (e.g., at the N′ of, at the C′ of, or within) the hinge region of the second antigen-binding polypeptide. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain.

In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the antibody comprises two heavy chains each comprising a hinge region, wherein the first cytokine or variant thereof is positioned at the hinge region (e.g., within the hinge region, or between the C-terminus of CH1 and the N-terminus of the hinge region) of a first heavy chain of the antibody, and wherein the second cytokine or variant thereof is positioned at the hinge region (e.g., within the hinge region, or between the C-terminus of CH1 and the N-terminus of the hinge region) of a second heavy chain of the antibody. In some embodiments, there is provided an immunocytokine comprising: a) an antibody specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the antibody comprises two heavy chains, wherein the first heavy chain comprises from N-terminus to C-terminus: a VH domain, optionally a CH1 domain, the first cytokine or variant thereof at a first hinge region, a CH2 domain, and optionally a CH3 domain; and wherein the second heavy chain comprises from N-terminus to C-terminus: a VH domain, optionally a CH1 domain, the second cytokine or variant thereof at a second hinge region, a CH2 domain, and optionally a CH3 domain. In some embodiments, there is provided an immunocytokine comprising: a) a full-length antibody specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the first cytokine or variant thereof is positioned at the hinge region (e.g., within the hinge region, or between the C-terminus of CH1 and the N-terminus of the hinge region) of a first heavy chain of the full-length antibody, and wherein the second cytokine or variant thereof is positioned at the hinge region (e.g., within the hinge region, or between the C-terminus of CH1 and the N-terminus of the hinge region) of a second heavy chain of the full-length antibody. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the first heavy chain and a first light chain of the antibody forms a first antigen-binding fragment (e.g., the first Fab) specifically recognizing a first target antigen, and the second heavy chain and a second light chain of the antibody forms a second antigen-binding fragment (e.g., the second Fab) specifically recognizing a second target antigen. In some embodiments, in the presence of binding of the antigen-binding protein (e.g., antibody such as full-length antibody, or ligand/receptor-hinge-Fc fusion protein) or antigen-binding fragment (e.g., ligand, receptor, VHH, scFv, Fab) to the target antigen (e.g., binding of the first antigen-binding fragment such as Fab or ligand to the first target antigen, and/or binding of the second antigen-binding fragment such as such as Fab or ligand to the second target antigen), the activity (binding affinity to corresponding cytokine receptor or subunit thereof, and/or biological activity) of the first and/or second cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen (neither antigen-binding fragment such as Fab or ligand binds to the target antigen). In some embodiments, in the absence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen (e.g., neither antigen-binding fragment such as Fab or ligand binds to the target antigen), the activity (binding affinity to corresponding cytokine receptor or subunit thereof, and/or biological activity) of the first and/or second cytokine or variant thereof positioned at the hinge region of each antigen-binding polypeptide (e.g., antibody heavy chain, or antigen-binding fragment-hinge-Fc polypeptide) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding first and/or second cytokine or variant thereof in a free state. In some embodiments, the first and/or second cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the two cytokines or variants thereof are the same. In some embodiments, the two cytokines or variants thereof are different. In some embodiments, the first cytokine or variant thereof is IL-2 or variant thereof (e.g., SEQ ID NO: 2). In some embodiments, the second cytokine or variant thereof is IL-12 or variant thereof (e.g., SEQ ID NO: 36). In some embodiments, the antigen-binding protein is an agonist. In some embodiments, the antigen-binding protein is an antagonist.

In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the antigen-binding protein comprises a antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), an wherein the first cytokine or variant thereof and the second cytokine or variant thereof are positioned in tandem (e.g., connected via an optional peptide linker) at the hinge region (e.g., within the hinge region, or between the C-terminus of CH1 and the N-terminus of the hinge region) of the antigen-binding polypeptide. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain.

In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the first cytokine or variant thereof and the second cytokine or variant thereof are positioned in tandem (e.g., connected via an optional peptide linker) at the hinge region (e.g., within the hinge region, or between the C-terminus of CH1 and the N-terminus of the hinge region) of the heavy chain. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, and wherein the antibody comprises a heavy chain comprising from N-terminus to C-terminus: a VH domain, optionally a CH1 domain, the first cytokine or variant thereof at the hinge region, optionally a linker (e.g., peptide linker), the second cytokine or variant thereof, a CH2 domain, and optionally a CH3 domain. In some embodiments, there is provided an immunocytokine comprising: a) a full-length antibody specifically recognizing a target antigen (e.g., CTLA-4, PD-L1, PD-L2, CD25, HER2, PD-1, CD3, CD4, CD123, or CD8); b) a first cytokine (e.g., IL-2, IFN-α (e.g., IFN-α2b), IFN-γ, IL-10, IL-12, or IL-23) or variant thereof, and c) a second cytokine (e.g., IL-2, IFN-α (e.g., IFN-α2b), IFN-γ, IL-10, IL-12, or IL-23) or variant thereof, wherein the first cytokine or variant thereof and the second cytokine or variant thereof are positioned in tandem (e.g., connected via an optional peptide linker) at the hinge region (e.g., within the hinge region, or between the C-terminus of CH1 and the N-terminus of the hinge region) of a heavy chain of the full-length antibody. In some embodiments, in the presence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the first and second cytokines or variants thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen. In some embodiments, in the absence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the first and second cytokines or variants thereof positioned at the hinge region of the antigen-binding polypeptide (e.g., antibody heavy chain, or antigen-binding fragment-hinge-Fc fusion protein) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of corresponding first and second cytokines or variants thereof in a free state. In some embodiments, the first and/or second cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific).

In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the two cytokines or variants thereof are the same. In some embodiments, the two cytokines or variants thereof are different. In some embodiments, the first cytokine or variant thereof is IL-2 or variant thereof (e.g., SEQ ID NO: 2). In some embodiments, the second cytokine or variant thereof is IL-12 or variant thereof (e.g., SEQ ID NO: 36). In some embodiments, the antigen-binding protein is an agonist. In some embodiments, the antigen-binding protein is an antagonist.

In some embodiments, the cytokine or variant thereof is a dimeric cytokine or variant thereof. In some embodiments, the cytokine or variant thereof is a homodimeric cytokine or variant thereof, such as IL-10 or IFN-γ. In some embodiments, the cytokine or variant thereof is a heterodimeric cytokine or variant thereof, such as IL-12 or IL-23. In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein both subunits of the dimeric cytokine or variant thereof are positioned in tandem (e.g., connected via an optional peptide linker such as any of SEQ ID NOs: 227-229) at (e.g., at the N′ of, at the C′ of, or within) the hinge region of the antigen-binding polypeptide. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein the antibody comprises a heavy chain comprising a hinge region, and wherein both subunits of the dimeric cytokine or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an immunocytokine comprising: a) an antibody specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, and wherein the antibody comprises a heavy chain comprising from N-terminus to C-terminus: a VH domain, optionally a CH1 domain, [a first subunit of the dimeric cytokine or variant thereof, optionally a linker (e.g., peptide linker), a second subunit of the dimeric cytokine or variant thereof] at a hinge region, a CH2 domain, and optionally a CH3 domain. In some embodiments, there is provided an immunocytokine comprising: a) a full-length antibody specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein both subunits of the dimeric cytokine or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, in the presence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen, the activity (binding affinity to corresponding cytokine receptor or subunit thereof, and/or biological activity) of the dimeric cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or ligand/receptor-hinge-Fc fusion protein) or antigen binding fragment (e.g., ligand, receptor, VHH, scFv, Fab) to the target antigen. In some embodiments, in the absence of binding of the antigen-binding protein (e.g., antibody such as full-length antibody, or ligand/receptor-hinge-Fc fusion protein) or antigen-binding fragment (e.g., ligand, receptor, VHH, scFv, Fab) to the target antigen, the activity (binding affinity to corresponding cytokine receptor or subunit thereof, and/or biological activity) of the dimeric cytokine or variant thereof positioned at the hinge region of the antigen-binding polypeptide (e.g., antibody heavy chain, or antigen-binding fragment-hinge-Fc fusion polypeptide) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding dimeric cytokine or variant thereof in a free state. In some embodiments, the dimeric cytokine or variant thereof is a dimeric cytokine variant, and the activity (binding affinity to corresponding cytokine receptor or subunit thereof, and/or biological activity) of the dimeric cytokine variant in a free state is no more than about 80% (such as no more than about any of 70%, 60%, 50%, 40%, 30%, 20%, 10%, or 5%) of that of a corresponding wildtype dimeric cytokine in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the cytokine or variant thereof is a homodimeric cytokine or variant thereof, such as IL-10 or IFN-γ. In some embodiments, the cytokine or variant thereof is a heterodimeric cytokine or variant thereof, such as IL-12 or IL-23. In some embodiments, the antigen-binding protein is an agonist. In some embodiments, the antigen-binding protein is an antagonist.

In some embodiments, there is provided an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein the antigen-binding protein comprises two antigen-binding polypeptides (e.g., antibody heavy chain, or antigen-binding fragment-hinge-Fc fusion polypeptide such as ligand/receptor-hinge-Fc fusion polypeptide) each comprising from N′ to C′: an antigen-binding fragment (e.g., ligand, receptor, VHH, scFv, or VH), a hinge region, and an Fc domain subunit or portion thereof (e.g., CH2+CH3, or CH2 only); wherein one subunit of the dimeric cytokine or variant thereof is positioned at (e.g., at the N′ of, at the C′ of, or within) the hinge region of the first antigen-binding polypeptide, and the other subunit of the dimeric cytokine or variant thereof is positioned at (e.g., at the N′ of, at the C′ of, or within) the hinge region of the second antigen-binding polypeptide. In some embodiments, the antigen-binding protein is an antibody specifically recognizing the target antigen, the antigen-binding polypeptide comprising the hinge region is a heavy chain of the antibody, and the cytokine or variant thereof is positioned at the hinge region of the heavy chain.

In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein the antibody comprises two heavy chains each comprising a hinge region, wherein one subunit of the dimeric cytokine or variant thereof is positioned at the hinge region (e.g., within the hinge region, or between the C-terminus of CH1 and the N-terminus of the hinge region) of the first heavy chain, and the other subunit of the dimeric cytokine or variant thereof is positioned at the hinge region (e.g., within the hinge region, or between the C-terminus of CH1 and the N-terminus of the hinge region) of the second heavy chain. In some embodiments, there is provided an immunocytokine comprising: a) an antibody specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein the antibody comprises two heavy chains, wherein the first heavy chain comprises from N-terminus to C-terminus: a VH domain, optionally a CH1 domain, a first subunit of the dimeric cytokine or variant thereof at a first hinge region, a CH2 domain, and optionally a CH3 domain, and wherein the second heavy chain comprises from N-terminus to C-terminus: a V_{H} domain, optionally a C_{H}1 domain, a second subunit of the dimeric cytokine or variant thereof at a second hinge region, a CH2 domain, and optionally a CH3 domain. In some embodiments, there is provided an immunocytokine comprising: a) a full-length antibody specifically recognizing a target antigen (e.g., CTLA-4, PD-L1, PD-L2, CD25, HER2, PD-1, CD3, CD4, CD123, or CD8); and b) a dimeric cytokine (e.g., IFN-γ, IL-10, IL-12, or IL-23) or variant thereof, wherein one subunit of the dimeric cytokine or variant thereof is positioned at the hinge region (e.g., within the hinge region, or between the C-terminus of CH1 and the N-terminus of the hinge region) of a first heavy chain of the full-length antibody, and the other subunit of the dimeric cytokine or variant thereof is positioned at the hinge region (e.g., between the C-terminus of CH1 and the N-terminus of the hinge region) of a second heavy chain of the full-length antibody. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the first heavy chain and a first light chain of the antibody forms a first antigen-binding fragment (e.g., the first Fab) specifically recognizing a first target antigen, and the second heavy chain and a second light chain of the antibody forms a second antigen-binding fragment (e.g., the second Fab) specifically recognizing a second target antigen. In some embodiments, in the presence of binding of the antigen-binding protein (e.g., antibody such as full-length antibody, or ligand/receptor-hinge-Fc fusion protein) or antigen-binding fragment (e.g., ligand, receptor, VHH, scFv, Fab) to the target antigen (e.g., binding of the first antigen-binding fragment such as Fab or ligand to the first target antigen, and/or binding of the second antigen-binding fragment such as Fab or ligand to the second target antigen), the activity (binding affinity to corresponding cytokine receptor or subunit thereof, and/or biological activity) of the dimeric cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or ligand/receptor-hinge-Fc fusion protein) or antigen-binding fragment (e.g., ligand, receptor, VHH, scFv, Fab) to the target antigen (e.g., neither antigen-binding fragment such as Fab or ligand binds to the target antigen). In some embodiments, in the absence of binding of the antigen-binding protein (e.g., antibody such as full-length antibody, or ligand/receptor-hinge-Fc fusion protein) or antigen-binding fragment (e.g., ligand, receptor, VHH, scFv, Fab) to the target antigen (e.g., neither antigen-binding fragment such as Fab or ligand binds to the target antigen), the activity (binding affinity to corresponding cytokine receptor or subunit thereof, and/or biological activity) of the dimeric cytokine or variant thereof with each subunit positioned at the hinge region of each antigen-binding polypeptide (e.g., antibody heavy chain, or antigen-binding fragment-hinge-Fc polypeptide) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding dimeric cytokine or variant thereof in a free state.

In some embodiments, the dimeric cytokine or variant thereof is a cytokine variant, and wherein the activity (binding affinity to corresponding cytokine receptor or subunit thereof, and/or biological activity) of the dimeric cytokine variant in a free state is no more than about 80% (such as no more than about any of 70%, 60%, 50%, 40%, 30%, 20%, 10%, or 5%) of that of a corresponding wildtype dimeric cytokine in a free state. In some embodiments, the cytokine or variant thereof is a homodimeric cytokine or variant thereof, such as IL-10 or IFN-γ. In some embodiments, the cytokine or variant thereof is a heterodimeric cytokine or variant thereof, such as IL-12 or IL-23. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist.

In some embodiments, the cytokine or variant thereof is IL-2 or variant thereof. In some embodiments, there is provided an IL-2 immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IL-2 or variant thereof (e.g., SEQ ID NO: 1 or 2), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein the IL-2 or variant thereof is positioned at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and the IL-2 or variant thereof is positioned at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IL-2 immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IL-2 or variant thereof (e.g., SEQ ID NO: 1 or 2), wherein the antibody comprises a heavy chain comprising a hinge region, and wherein the IL-2 or variant thereof is positioned at the hinge region (e.g., within the hinge region, or between the C-terminus of CH1 and the N-terminus of the hinge region) of the heavy chain. In some embodiments, there is provided an IL-2 immunocytokine comprising: a) an antibody specifically recognizing a target antigen (e.g., CTLA-4, PD-L1, PD-L2, CD25, HER2, PD-1, CD3, CD4, CD123, or CD8); and b) an anti-IL-2 or variant thereof (e.g., SEQ ID NO: 1 or 2), wherein the antibody comprises a heavy chain, and wherein the heavy chain comprises from N-terminus to C-terminus: a VH domain, optionally a CH1 domain, the IL-2 or variant thereof at a hinge region, a CH2 domain, and optionally a CH3 domain. In some embodiments, there is provided an IL-2 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-2 or variant thereof (e.g., SEQ ID NO: 1 or 2), wherein the IL-2 or variant thereof is positioned at the hinge region (e.g., within the hinge region, or between the C-terminus of CH1 and the N-terminus of the hinge region) of a heavy chain of the full-length antibody. In some embodiments, there is provided an IL-2 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-2 variant comprising one or more mutations at a position selected from the group consisting of L18, Q22, F24, K35, R38, F42, K43, E61, and P65 relative to a wildtype IL-2 comprising the sequence of SEQ ID NO: 1, wherein the IL-2 variant is positioned at the hinge region (e.g., within the hinge region, or between the C-terminus of CH1 and the N-terminus of the hinge region) of a heavy chain of the full-length antibody. In some embodiments, there is provided an IL-2 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-2 variant comprising one or more mutations selected from the group consisting of F24A, R38D, K43E, E61R, and P65L relative to a wildtype IL-2 comprising the sequence of SEQ ID NO: 1, wherein the IL-2 variant is positioned at the hinge region (e.g., within the hinge region, or between the C-terminus of CH1 and the N-terminus of the hinge region) of a heavy chain of the full-length antibody. In some embodiments, there is provided an IL-2 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-2 variant comprising an R38D/K43E/E61R mutation relative to a wildtype IL-2 comprising the sequence of SEQ ID NO: 1, wherein the IL-2 variant is positioned at the hinge region (e.g., within the hinge region, or between the C-terminus of CH1 and the N-terminus of the hinge region) of a heavy chain of the full-length antibody. In some embodiments, there is provided an IL-2 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-2 variant comprising the sequence of SEQ ID NO: 2, wherein the IL-2 variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IL-2 variant in a free state is no more than about 80% (such as no more than about any of 70%, 60%, 50%, 40%, 30%, 20%, 10%, or 5%) of that of a corresponding wildtype IL-2 in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific).

In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IL-2 immunocytokine (“IL-2/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-HER2 immunocytokine comprising: a) a full-length antibody specifically recognizing HER2; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154. In some embodiments, the IL-2 variant comprises the sequence of SEQ ID NO: 2. In some embodiments, the heavy chain comprising the IL-2 variant positioned at the hinge region comprises the sequence of SEQ ID NO: 156. In some embodiments, there is provided an IL-2/anti-HER2 immunocytokine comprising: two light chains comprising the sequence of SEQ ID NO: 154, one heavy chain comprising an IL-2 variant positioned at the hinge region comprising the sequence of SEQ ID NO: 156, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IL-2 immunocytokine (“IL-2/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-CD3 immunocytokine comprising: a) a full-length antibody specifically recognizing CD3; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, the IL-2 variant comprises the sequence of SEQ ID NO: 2. In some embodiments, the heavy chain comprising the IL-2 variant positioned at the hinge region comprises the sequence of SEQ ID NO: 96. In some embodiments, there is provided an IL-2/anti-CD3 immunocytokine comprising: two light chains comprising the sequence of SEQ ID NO: 94, one heavy chain comprising an IL-2 variant positioned at the hinge region comprising the sequence of SEQ ID NO: 96, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-1 antibody. Thus in some embodiments, there is provided an IL-2 immunocytokine (“IL-2/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., any of SEQ ID NOs: 40-47, 50-52, and 55-59)(e.g., within the hinge region, or between the C-terminus of CH1 and the N-terminus of the hinge region) of the heavy chain. In some embodiments, there is provided an IL-2/anti-PD-1 immunocytokine comprising: a) a full-length antibody specifically recognizing PD-1; and b) an IL-2 or variant thereof (e.g., SEQ ID NO: 1 or 2), wherein the IL-2 or variant thereof is positioned at the hinge region (e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, the IL-2 variant comprises the sequence of SEQ ID NO: 2. In some embodiments, the heavy chain comprising the IL-2 variant positioned at the hinge region comprises the sequence of SEQ ID NO: 108. In some embodiments, there is provided an IL-2/anti-PD-1 immunocytokine comprising: two light chains comprising the sequence of SEQ ID NO: 106, one heavy chain comprising an IL-2 variant positioned at the hinge region comprising the sequence of SEQ ID NO: 108, and one heavy chain comprises the sequence of SEQ ID NO: 107. In some embodiments, in the presence of binding of the antibody (or antigen-binding fragment) to PD-1, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-1. In some embodiments, in the absence of binding of the antibody (or antigen-binding fragment) to PD-1, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific).

In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD4 antibody. Thus in some embodiments, there is provided an IL-2 immunocytokine (“IL-2/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-CD4 immunocytokine comprising: a) a full-length antibody specifically recognizing CD4; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, the IL-2 variant comprises the sequence of SEQ ID NO: 2. In some embodiments, the heavy chain comprising the IL-2 variant positioned at the hinge region comprises the sequence of SEQ ID NO: 79. In some embodiments, there is provided an IL-2/anti-CD4 immunocytokine comprising: two light chains comprising the sequence of SEQ ID NO: 77, one heavy chain comprising an IL-2 variant positioned at the hinge region comprising the sequence of SEQ ID NO: 79, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD8 antibody. Thus in some embodiments, there is provided an IL-2 immunocytokine (“IL-2/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-CD8 immunocytokine comprising: a) a full-length antibody specifically recognizing CD8; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CD8 antibody comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, the IL-2 variant comprises the sequence of SEQ ID NO: 2. In some embodiments, the heavy chain comprising the IL-2 variant positioned at the hinge region comprises the sequence of SEQ ID NO: 119. In some embodiments, there is provided an IL-2/anti-CD8 immunocytokine comprising: two light chains comprising the sequence of SEQ ID NO: 117, one heavy chain comprising an IL-2 variant positioned at the hinge region comprising the sequence of SEQ ID NO: 119, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IL-2 immunocytokine (“IL-2/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-CTLA-4 immunocytokine comprising: a) a full-length antibody specifically recognizing CTLA-4; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, the IL-2 variant comprises the sequence of SEQ ID NO: 2. In some embodiments, the heavy chain comprising the IL-2 variant positioned at the hinge region comprises the sequence of SEQ ID NO: 131. In some embodiments, there is provided an IL-2/anti-CTLA-4 immunocytokine comprising: two light chains comprising the sequence of SEQ ID NO: 129, one heavy chain comprising an IL-2 variant positioned at the hinge region comprising the sequence of SEQ ID NO: 131, and one heavy chain comprises the sequence of SEQ ID NO: 130.

In some embodiments, in the presence of binding of the antibody (or antigen-binding fragment) to CTLA-4, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IL-2 immunocytokine (“IL-2/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, between the C-terminus of CH1 and the N-terminus of the hinge region) of the heavy chain. In some embodiments, there is provided an IL-2/anti-PD-L1 immunocytokine comprising: a) a full-length antibody specifically recognizing PD-L1; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, the IL-2 variant comprises the sequence of SEQ ID NO: 2. In some embodiments, the heavy chain comprising the IL-2 variant positioned at the hinge region comprises the sequence of SEQ ID NO: 144. In some embodiments, there is provided an IL-2/anti-PD-L1 immunocytokine comprising: two light chains comprising the sequence of SEQ ID NO: 140, one heavy chain comprising an IL-2 variant positioned at the hinge region comprising the sequence of SEQ ID NO: 144, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IL-2 immunocytokine (“IL-2/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IL-2 or variant thereof (e.g., SEQ ID NO: 1 or 2), wherein the antibody comprises a heavy chain comprising a hinge region (e.g., any of SEQ ID NOs: 40-47, 50-52, and 55-59), and wherein the IL-2 or variant thereof is positioned at the hinge region (e.g., within the hinge region, or between the C-terminus of CH1 and the N-terminus of the hinge region) of the heavy chain. In some embodiments, there is provided an IL-2/anti-CD25 immunocytokine comprising: a) a full-length antibody specifically recognizing CD25; and b) an IL-2 or variant thereof (e.g., SEQ ID NO: 1 or 2), wherein the IL-2 or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, the IL-2 variant comprises the sequence of SEQ ID NO: 2. In some embodiments, the heavy chain comprising the IL-2 variant positioned at the hinge region comprises the sequence of SEQ ID NO: 169. In some embodiments, there is provided an IL-2/anti-CD25 immunocytokine comprising: two light chains comprising the sequence of SEQ ID NO: 165, one heavy chain comprising an IL-2 variant positioned at the hinge region comprising the sequence of SEQ ID NO: 169, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IL-2 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IL-2 immunocytokine (“IL-2/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region, and an Fc domain subunit or portion thereof; and b) an IL-2 or variant thereof (e.g., SEQ ID NO: 1 or 2), and wherein the IL-2 or variant thereof is positioned at the hinge region (e.g., any of SEQ ID NOs: 40-47, 50-52, and 55-59)(e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, the IL-2 variant comprises the sequence of SEQ ID NO: 2. In some embodiments, the PD-L2-hinge-Fc fusion polypeptide comprising the IL-2 variant positioned at the hinge region comprises the sequence of SEQ ID NO: 180. In some embodiments, there is provided an IL-2/PD-L2-Fc immunocytokine comprising: a PD-L2-hinge-Fc fusion polypeptide comprising an IL-2 variant positioned at the hinge region comprises the sequence of SEQ ID NO: 180, and a PD-L2-hinge-Fc fusion polypeptide comprises the sequence of SEQ ID NO: 179. In some embodiments, in the presence of binding of the PD-L2 ligand to PD-1, the activity (binding affinity to corresponding cytokine receptor or subunit thereof, and/or biological activity) of the IL-2 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IL-2 cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-2 cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the cytokine or variant thereof is IFN-α (e.g., IFN-α2b) or variant thereof. In some embodiments, there is provided an IFN-α (e.g., IFN-α2b) immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IFN-α (e.g., IFN-α2b) or variant thereof (e.g., any of SEQ ID NOs: 3-9), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein the IFN-α or variant thereof is positioned at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and the IFN-α or variant thereof is positioned at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IFN-α (e.g., IFN-α2b) immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IFN-α (e.g., IFN-α2b) or variant thereof, wherein the antibody comprises a heavy chain comprising a hinge region, and wherein the IFN-α (e.g., IFN-α2b) or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b) immunocytokine comprising: a) an antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) or variant thereof, wherein the antibody comprises a heavy chain, and wherein the heavy chain comprises from N-terminus to C-terminus: a VH domain, optionally a CH1 domain, the IFN-α (e.g., IFN-α2b) or variant thereof at the hinge region, a CH2 domain, and optionally a CH3 domain. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) or variant thereof, wherein the IFN-α (e.g., IFN-α2b) or variant thereof is positioned at the hinge region (e.g., within the hinge region, or between the C-terminus of CH1 and the N-terminus of the hinge region) of a heavy chain of the full-length antibody. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) variant comprising one or more mutations at a position selected from the group consisting of R22, L26, F27, L30, K31, D32, R33, H34, D35, F36, S68, T79, K83, Y85, Y89, R120, K121, Y122, Q124, Y129, K131, E132, R144, and E146 relative to an IFN-α comprising the sequence of SEQ ID NO: 3, wherein the IFN-α (e.g., IFN-α2b) variant is positioned at the hinge region (e.g., within the hinge region, or between the C-terminus of CH1 and the N-terminus of the hinge region) of a heavy chain of the full-length antibody. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) variant comprising one or more mutations selected from the group consisting of L30A, K31A, D32A, R33A, H34A, and D35A relative to an IFN-α comprising the sequence of SEQ ID NO: 3, wherein the IFN-α (e.g., IFN-α2b) variant is positioned at the hinge region (e.g., within the hinge region, or between the C-terminus of CH1 and the N-terminus of the hinge region) of a heavy chain of the full-length antibody. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) variant comprising the sequence of any of SEQ ID NOs: 4-9, wherein the IFN-α (e.g., IFN-α2b) variant is positioned at the hinge region (e.g., within the hinge region, or between the C-terminus of CH1 and the N-terminus of the hinge region) of a heavy chain of the full-length antibody. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) variant comprising an L30A mutation relative to an IFN-α comprising the sequence of SEQ ID NO: 3, wherein the IFN-α (e.g., IFN-α2b) variant is positioned at the hinge region (e.g., within the hinge region, or between the C-terminus of CH1 and the N-terminus of the hinge region) of a heavy chain of the full-length antibody. In some embodiments, there is provided an IFN-α (e.g., IFN-α2b) immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-α (e.g., IFN-α2b) variant comprising the sequence of SEQ ID NO: 4, wherein the IFN-α (e.g., IFN-α2b) variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IFN-α (e.g., IFN-α2b) variant in a free state is no more than about 80% (such as no more than about any of 70%, 60%, 50%, 40%, 30%, 20%, 10%, or 5%) of that of a corresponding wildtype IFN-α (e.g., IFN-α2b) in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IFN-α (e.g., IFN-α2b) immunocytokine (“IL-2/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-HER2 immunocytokine comprising: a) a full-length antibody specifically recognizing HER2; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154. In some embodiments, the IFN-α (e.g., IFN-α2b) variant comprises the sequence of SEQ ID NO: 4. In some embodiments, the heavy chain comprising the IFN-α variant positioned at the hinge region comprises the sequence of SEQ ID NO: 157. In some embodiments, there is provided an IFN-α (e.g., IFN-α2b)/anti-HER2 immunocytokine comprising: two light chains comprising the sequence of SEQ ID NO: 154, one heavy chain comprising an IFN-α variant positioned at the hinge region comprising the sequence of SEQ ID NO: 157, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IFN-α cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IFN-α cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IFN-α (e.g., IFN-α2b) immunocytokine (“IL-2/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-CD3 immunocytokine comprising: a) a full-length antibody specifically recognizing CD3; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, the IFN-α (e.g., IFN-α2b) variant comprises the sequence of SEQ ID NO: 4. In some embodiments, the heavy chain comprising the IFN-α variant positioned at the hinge region comprises the sequence of SEQ ID NO: 97. In some embodiments, there is provided an IFN-α (e.g., IFN-α2b)/anti-CD3 immunocytokine comprising: two light chains comprising the sequence of SEQ ID NO: 94, one heavy chain comprising an IFN-α variant positioned at the hinge region comprising the sequence of SEQ ID NO: 97, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-1 antibody. Thus in some embodiments, there is provided an IFN-α (e.g., IFN-α2b) immunocytokine (“IFN-α/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α (e.g., IFN-α2b) or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-PD-1 immunocytokine comprising: a) a full-length antibody specifically recognizing PD-1; and b) an IFN-α (e.g., IFN-α2b) or variant thereof (e.g., any of SEQ ID NOs: 3-9), wherein the IFN-α (e.g., IFN-α2b) or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, the IFN-α (e.g., IFN-α2b) variant comprises the sequence of SEQ ID NO: 4. In some embodiments, the heavy chain comprising the IFN-α (e.g., IFN-α2b) variant positioned at the hinge region comprises the sequence of SEQ ID NO: 109. In some embodiments, there is provided an IFN-α (e.g., IFN-α2b)/anti-PD-1 immunocytokine comprising: two light chains comprising the sequence of SEQ ID NO: 106, one heavy chain comprising an IFN-α variant positioned at the hinge region comprising the sequence of SEQ ID NO: 109, and one heavy chain comprises the sequence of SEQ ID NO: 107.

In some embodiments, in the presence of binding of the antibody (or antigen-binding fragment) to PD-1, the activity (binding affinity to corresponding cytokine receptor or subunit thereof, and/or biological activity) of the IFN-α (e.g., IFN-α2b) or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-1. In some embodiments, in the absence of binding of the antibody (or antigen-binding fragment) to PD-1, the activity (binding affinity to corresponding cytokine receptor or subunit thereof, and/or biological activity) of the IFN-α (e.g., IFN-α2b) or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α (e.g., IFN-α2b) or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD4 antibody. Thus in some embodiments, there is provided an IFN-α (e.g., IFN-α2b) immunocytokine (“IFN-α/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-CD4 immunocytokine comprising: a) a full-length antibody specifically recognizing CD4; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, the IFN-α (e.g., IFN-α2b) variant comprises the sequence of SEQ ID NO: 4. In some embodiments, the heavy chain comprising the IFN-α variant positioned at the hinge region comprises the sequence of SEQ ID NO: 80. In some embodiments, there is provided an IFN-α (e.g., IFN-α2b)/anti-CD4 immunocytokine comprising: two light chains comprising the sequence of SEQ ID NO: 77, one heavy chain comprising an IFN-α variant positioned at the hinge region comprising the sequence of SEQ ID NO: 80, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IFN-α (e.g., IFN-α2b) cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD8 antibody. Thus in some embodiments, there is provided an IFN-α (e.g., IFN-α2b) immunocytokine (“IFN-α/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-CD8 immunocytokine comprising: a) a full-length antibody specifically recognizing CD8; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CD8 antibody comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, the IFN-α (e.g., IFN-α2b) variant comprises the sequence of SEQ ID NO: 4. In some embodiments, the heavy chain comprising the IFN-α variant positioned at the hinge region comprises the sequence of SEQ ID NO: 120. In some embodiments, there is provided an IFN-α (e.g., IFN-α2b)/anti-CD8 immunocytokine comprising: two light chains comprising the sequence of SEQ ID NO: 117, one heavy chain comprising an IFN-α variant positioned at the hinge region comprising the sequence of SEQ ID NO: 120, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IFN-α (e.g., IFN-α2b) immunocytokine (“IFN-α/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-CTLA-4 immunocytokine comprising: a) a full-length antibody specifically recognizing CTLA-4; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, the IFN-α (e.g., IFN-α2b) variant comprises the sequence of SEQ ID NO: 4. In some embodiments, the heavy chain comprising the IFN-α variant positioned at the hinge region comprises the sequence of SEQ ID NO: 132. In some embodiments, there is provided an IFN-α (e.g., IFN-α2b)/anti-CTLA-4 immunocytokine comprising: two light chains comprising the sequence of SEQ ID NO: 129, one heavy chain comprising an IFN-α variant positioned at the hinge region comprising the sequence of SEQ ID NO: 132, and one heavy chain comprises the sequence of SEQ ID NO: 130. In some embodiments, in the presence of binding of the antibody (or antigen-binding fragment) to CTLA-4, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IFN-α (e.g., IFN-α2b) immunocytokine (“IFN-α/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, between the C-terminus of CH1 and the N-terminus of the hinge region) of the heavy chain. In some embodiments, there is provided an IFN-α (e.g., IFN-α2b)/anti-PD-L1 immunocytokine comprising: a) a full-length antibody specifically recognizing PD-L1; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, the IFN-α (e.g., IFN-α2b) variant comprises the sequence of SEQ ID NO: 4. In some embodiments, the heavy chain comprising the IFN-α (e.g., IFN-α2b) variant positioned at the hinge region comprises the sequence of SEQ ID NO: 145. In some embodiments, there is provided an IFN-α (e.g., IFN-α2b)/anti-PD-L1 immunocytokine comprising: two light chains comprising the sequence of SEQ ID NO: 140, one heavy chain comprising an IFN-α variant positioned at the hinge region comprising the sequence of SEQ ID NO: 145, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IFN-α (e.g., IFN-α2b) immunocytokine (“IFN-α/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IFN-α (e.g., IFN-α2b) or variant thereof (e.g., any of SEQ ID NOs: 3-9), wherein the antibody comprises a heavy chain comprising a hinge region (e.g., any of SEQ ID NOs: 40-47, 50-52, and 55-59), and wherein the IFN-α or variant thereof is positioned at the hinge region (e.g., within the hinge region, or between the C-terminus of CH1 and the N-terminus of the hinge region) of the heavy chain. In some embodiments, there is provided an IFN-α (e.g., IFN-α2b)/anti-CD25 immunocytokine comprising: a) a full-length antibody specifically recognizing CD25; and b) an IFN-α (e.g., IFN-α2b) or variant thereof (e.g., any of SEQ ID NOs: 3-9), wherein the IFN-α or variant thereof is positioned at the hinge region (e.g., any of SEQ ID NOs: 40-47, 50-52, and 55-59)(e.g., within the hinge region, between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, the IFN-α (e.g., IFN-α2b) variant comprises the sequence of SEQ ID NO: 4. In some embodiments, the heavy chain comprising the IFN-α (e.g., IFN-α2b) variant positioned at the hinge region comprises the sequence of SEQ ID NO: 170. In some embodiments, there is provided an IFN-α (e.g., IFN-α2b)/anti-CD25 immunocytokine comprising: two light chains comprising the sequence of SEQ ID NO: 165, one heavy chain comprising an IFN-α variant positioned at the hinge region comprising the sequence of SEQ ID NO: 170, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IFN-α (e.g., IFN-α2b) cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IFN-α (e.g., IFN-α2b) immunocytokine (“IFN-α/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region (e.g., any of SEQ ID NOs: 40-47, 50-52, and 55-59), and an Fc domain subunit or portion thereof; and b) an IFN-α (e.g., IFN-α2b) or variant thereof (e.g., any of SEQ ID NOs: 3-9), and wherein the IFN-α or variant thereof is positioned at the hinge region (e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, the IFN-α (e.g., IFN-α2b) variant comprises the sequence of SEQ ID NO: 4. In some embodiments, the PD-L2-hinge-Fc fusion polypeptide comprising the IFN-α (e.g., IFN-α2b) variant positioned at the hinge region comprises the sequence of SEQ ID NO: 182. In some embodiments, there is provided an IFN-α/PD-L2-Fc immunocytokine comprising: a PD-L2-hinge-Fc fusion polypeptide comprising an IFN-α variant positioned at the hinge region comprises the sequence of SEQ ID NO: 182, and a PD-L2-hinge-Fc fusion polypeptide comprises the sequence of SEQ ID NO: 181. In some embodiments, in the presence of binding of the PD-L2 ligand to PD-1, the activity (binding affinity to corresponding cytokine receptor or subunit thereof, and/or biological activity) of the IFN-α (e.g., IFN-α2b) cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IFN-α (e.g., IFN-α2b) cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-α cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the cytokine or variant thereof is IFN-γ or variant thereof. In some embodiments, there is provided an IFN-γ immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IFN-γ or variant thereof (e.g., SEQ ID NO: 18 or 19), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IFN-γ immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IFN-γ or variant thereof (e.g., SEQ ID NO: 18 or 19), wherein the antibody comprises a heavy chain comprising a hinge region, and wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ immunocytokine comprising: a) an antibody specifically recognizing a target antigen (e.g., CTLA-4, PD-L1, PD-L2, CD25, HER2, PD-1, CD3, CD4, CD123, or CD8); and b) an IFN-γ or variant thereof (e.g., SEQ ID NO: 18 or 19), wherein the antibody comprises a heavy chain comprising from N-terminus to C-terminus: a VH domain, optionally a CH1 domain, [a first subunit of IFN-γ or variant thereof (e.g., any of SEQ ID NOs: 10⁻¹⁷), optionally a linker (e.g., peptide linker), a second subunit of IFN-γ or variant thereof (e.g., any of SEQ ID NOs: 10⁻¹⁷)] at a hinge region, a CH2 domain, and optionally a CH3 domain. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ or variant thereof (e.g., SEQ ID NO: 18 or 19), wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant comprising one or more mutations within one or both IFN-γ subunits at a position selected from the group consisting of V5, S20, D21, V22, A23, D24, N25, G26, H111, and Q115 relative to a wildtype IFN-γ subunit comprising the sequence of SEQ ID NO: 10, wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant comprising one or more mutations within one or both IFN-γ subunits selected from the group consisting of S20A, D21A, D21K, V22A, A23S, A23E, A23Q, A23V, D24A, D24E, N25A, N25K, and H111D relative to a wildtype IFN-γ subunit comprising the sequence of SEQ ID NO: 10, wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant comprising one or more mutations within one or both IFN-γ subunits selected from the group consisting of S20A/D21A, D21K, V22A/A23S, D24A/N25A, A23E/D24E/N25K, A23Q, and A23V relative to a wildtype IFN-γ subunit comprising the sequence of SEQ ID NO: 10, wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant, wherein one or both subunits of the IFN-γ variant comprises the sequence of any of SEQ ID NOs: 11-17, wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant comprising an A23V mutation within one or both IFN-γ subunits relative to a wildtype IFN-γ subunit comprising the sequence of SEQ ID NO: 10, wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant, wherein one or both subunits of the IFN-γ variant comprises the sequence of SEQ ID NO: 13, and wherein both subunits of the IFN-γ variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, the two subunits of the IFN-γ or variant thereof are connected by a linker (e.g., any of SEQ ID NOs: 227-229). In some embodiments, there is provided an IFN-γ immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IFN-γ variant comprising the sequence of SEQ ID NO: 19, wherein the IFN-γ variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IFN-γ variant in a free state is no more than about 80% (such as no more than about any of 70%, 60%, 50%, 40%, 30%, 20%, 10%, or 5%) of that of a corresponding wildtype IFN-γ in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IFN-γ immunocytokine (“IFN-γ/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-HER2 immunocytokine comprising: a) a full-length antibody specifically recognizing HER2; and b) an IFN-γ or variant thereof (e.g., SEQ ID NO: 18 or 19), wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 158. In some embodiments, there is provided an IFN-γ/anti-HER2 immunocytokine comprising: two light chains comprising the sequence of SEQ ID NO: 154, one heavy chain comprising an IFN-γ variant positioned at the hinge region comprising the sequence of SEQ ID NO: 158, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IFN-γ immunocytokine (“IFN-γ/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-CD3 immunocytokine comprising: a) a full-length antibody specifically recognizing CD3; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 98. In some embodiments, there is provided an IFN-γ/anti-CD3 immunocytokine comprising: two light chains comprising the sequence of SEQ ID NO: 94, one heavy chain comprising an IFN-γ variant positioned at the hinge region comprising the sequence of SEQ ID NO: 98, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-PD-1 antibody. Thus in some embodiments, there is provided an IFN-γ immunocytokine (“IFN-γ/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-PD-1 immunocytokine comprising: a) a full-length antibody specifically recognizing PD-1; and b) an IFN-γ or variant thereof (e.g., SEQ ID NO: 18 or 19), wherein both subunits (e.g., any of SEQ ID NOs: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 110. In some embodiments, there is provided an IFN-γ/anti-PD-1 immunocytokine comprising: two light chains comprising the sequence of SEQ ID NO: 106, one heavy chain comprising an IFN-γ variant positioned at the hinge region comprising the sequence of SEQ ID NO: 110, and one heavy chain comprises the sequence of SEQ ID NO: 107. In some embodiments, in the presence of binding of the antibody (or antigen-binding fragment) to PD-1, the activity (binding affinity to corresponding cytokine receptor or subunit thereof, and/or biological activity) of the IFN-γ or variant thereof increases at least about 20% (such as at least about any of 30%, 40%, 50%, 60%, 70%, 80%, 90%, 100%, 200%, 300%, 400%, 500%, or more) compared to that in the absence of binding of the antibody to PD-1 (or antigen-binding fragment). In some embodiments, in the absence of binding of the antibody (or antigen-binding fragment) to PD-1, the activity (binding affinity to corresponding cytokine receptor or subunit thereof, and/or biological activity) of the IFN-γ or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-CD4 antibody. Thus in some embodiments, there is provided an IFN-γ immunocytokine (“IFN-γ/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-CD4 immunocytokine comprising: a) a full-length antibody specifically recognizing CD4; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 81. In some embodiments, there is provided an IFN-γ/anti-CD4 immunocytokine comprising: two light chains comprising the sequence of SEQ ID NO: 77, one heavy chain comprising an IFN-γ variant positioned at the hinge region comprising the sequence of SEQ ID NO: 81, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IFN-γ or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IFN-γ or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IFN-γ or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD8 antibody. Thus in some embodiments, there is provided an IFN-γ immunocytokine (“IFN-γ/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-CD8 immunocytokine comprising: a) a full-length antibody specifically recognizing CD8; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD8 antibody comprises VH-CDR1, VH-CDR2, and VH-CDR3 of a V_{H} comprising the sequence of SEQ ID NO: 114, and VL-CDR1, VL-CDR2, and VL-CDR3 of a V_{L} comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 121. In some embodiments, there is provided an IFN-γ/anti-CD8 immunocytokine comprising: two light chains comprising the sequence of SEQ ID NO: 117, one heavy chain comprising an IFN-γ variant positioned at the hinge region comprising the sequence of SEQ ID NO: 121, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IFN-γ immunocytokine (“IFN-γ/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-CTLA-4 immunocytokine comprising: a) a full-length antibody specifically recognizing CTLA-4; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 133. In some embodiments, there is provided an IFN-γ/anti-CTLA-4 immunocytokine comprising: two light chains comprising the sequence of SEQ ID NO: 129, one heavy chain comprising an IFN-γ variant positioned at the hinge region comprising the sequence of SEQ ID NO: 133, and one heavy chain comprises the sequence of SEQ ID NO: 130. In some embodiments, in the presence of binding of the antibody (or antigen-binding fragment) to CTLA-4, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IFN-γ immunocytokine (“IFN-γ/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-PD-L1 immunocytokine comprising: a) a full-length antibody specifically recognizing PD-L1; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 146. In some embodiments, there is provided an IFN-γ/anti-PD-L1 immunocytokine comprising: two light chains comprising the sequence of SEQ ID NO: 140, one heavy chain comprising an IFN-γ variant positioned at the hinge region comprising the sequence of SEQ ID NO: 146, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IFN-γ immunocytokine (“IFN-γ/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IFN-γ or variant thereof (e.g., SEQ ID NO: 18 or 19), wherein the antibody comprises a heavy chain comprising a hinge region (e.g., any of SEQ ID NOs: 40-47, 50-52, and 55-59), and wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IFN-γ/anti-CD25 immunocytokine comprising: a) a full-length antibody specifically recognizing CD25; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, one or both subunits of the IFN-γ variant comprises the sequence of SEQ ID NO: 13. In some embodiments, the IFN-γ variant comprises the sequence of SEQ ID NO: 19. In some embodiments, the heavy chain comprising the IFN-γ variant positioned at the hinge region comprises the sequence of SEQ ID NO: 171. In some embodiments, there is provided an IFN-γ/anti-CD25 immunocytokine comprising: two light chains comprising the sequence of SEQ ID NO: 165, one heavy chain comprising an IFN-γ variant positioned at the hinge region comprising the sequence of SEQ ID NO: 171, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IFN-γ cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IFN-γ immunocytokine (“IFN-γ/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region (e.g., any of SEQ ID NOs: 40-47, 50-52, and 55-59), and an Fc domain subunit or portion thereof; and b) an IFN-γ or variant thereof (e.g., SEQ ID NO: 18 or 19), wherein both subunits (e.g., any of SEQ ID NO: 10⁻¹⁷) of the IFN-γ or variant thereof are positioned in tandem (e.g., connected via an optional peptide linker such as any of SEQ ID NOs: 227-229) at the hinge region (e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, one or both subunits of the IFN-γ variant comprises the sequence of SEQ ID NO: 13. In some embodiments, the IFN-γ variant comprises the sequence of SEQ ID NO: 19. In some embodiments, the PD-L2-hinge-Fc fusion polypeptide comprising the IFN-γ variant positioned at the hinge region comprises the sequence of SEQ ID NO: 183. In some embodiments, there is provided an IFN-γ/PD-L2-Fc immunocytokine comprising: a PD-L2-hinge-Fc fusion polypeptide comprising an IFN-γ variant positioned at the hinge region comprises the sequence of SEQ ID NO: 183, and a PD-L2-hinge-Fc fusion polypeptide comprises the sequence of SEQ ID NO: 181. In some embodiments, in the presence of binding of the PD-L2 ligand to PD-1, the activity (binding affinity to corresponding cytokine receptor or subunit thereof, and/or biological activity) of the IFN-γ cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IFN-γ cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IFN-γ cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the cytokine or variant thereof is IL-10 or variant thereof. In some embodiments, there is provided an IL-10 immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IL-10 or variant thereof (e.g., SEQ ID NO: 27 or 28), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein both subunits (e.g., any of SEQ ID NOs: 20-26) of the IL-10 or variant thereof are positioned in tandem (e.g., connected via an optional peptide linker such as any of SEQ ID NOs: 227-229) at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and both subunits (e.g., any of SEQ ID NOs: 20-26) of the IL-10 or variant thereof are positioned in tandem (e.g., connected via an optional peptide linker such as any of SEQ ID NOs: 227-229) at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IL-10 immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IL-10 or variant thereof (e.g., SEQ ID NO: 27 or 28), wherein the antibody comprises a heavy chain comprising a hinge region, and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10 immunocytokine comprising: a) an antibody specifically recognizing a target antigen (e.g., CTLA-4, PD-L1, PD-L2, CD25, HER2, PD-1, CD3, CD4, CD123, or CD8); and b) an IL-10 or variant thereof (e.g., SEQ ID NO: 27 or 28), wherein the antibody comprises a heavy chain comprising from N-terminus to C-terminus: a VH domain, optionally a CH1 domain, [a first subunit of IL-10 or variant thereof (e.g., any of SEQ ID NOs: 20-26), optionally a linker (e.g., peptide linker), a second subunit of IL-10 or variant thereof (e.g., any of SEQ ID NOs: 20-26)] at a hinge region, a CH2 domain, and optionally a CH3 domain. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 or variant thereof (e.g., SEQ ID NO: 27 or 28), wherein both subunits of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant comprising one or more mutations within one or both IL-10 subunits at a position selected from the group consisting of N21, M22, R24, D25, L26, R27, D28, A29, F30, S31, R32, H90, and S93 relative to a wildtype IL-10 subunit comprising the sequence of SEQ ID NO: 20, wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant comprising one or more mutations within one or both IL-10 subunits selected from the group consisting of R24A, D25A, L26A, R27A, D28A, A29S, F30A, S31A, and R32A relative to a wildtype IL-10 subunit comprising the sequence of SEQ ID NO: 20, wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant comprising one or more mutations within one or both IL-10 subunits selected from the group consisting of R24A, D25A/L26A, R27A, D28A/A29S, F30A/S31A, and R32A relative to a wildtype IL-10 subunit comprising the sequence of SEQ ID NO: 20, wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant, wherein one or both subunits of the IL-10 variant comprises the sequence of any of SEQ ID NOs: 21-26, and wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant comprising an R27A mutation within one or both IL-10 subunits relative to a wildtype IL-10 subunit comprising the sequence of SEQ ID NO: 20, wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant, wherein one or both subunits of the IL-10 variant comprises the sequence of SEQ ID NO: 23, and wherein both subunits of the IL-10 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, the two subunits of the IL-10 or variant thereof are connected by a linker. In some embodiments, there is provided an IL-10 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-10 variant comprising the sequence of SEQ ID NO: 28, wherein the IL-10 variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IL-10 variant in a free state is no more than about 80% (such as no more than about any of 70%, 60%, 50%, 40%, 30%, 20%, 10%, or 5%) of that of a corresponding wildtype IL-10 in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IL-10 immunocytokine (“IL-10/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-HER2 immunocytokine comprising: a) a full-length antibody specifically recognizing HER2; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 159. In some embodiments, there is provided an IL-10/anti-HER2 immunocytokine comprising: two light chains comprising the sequence of SEQ ID NO: 154, one heavy chain comprising an IL-10 variant positioned at the hinge region comprising the sequence of SEQ ID NO: 159, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IL-10 immunocytokine (“IL-10/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-CD3 immunocytokine comprising: a) a full-length antibody specifically recognizing CD3; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 99. In some embodiments, there is provided an IL-10/anti-CD3 immunocytokine comprising: two light chains comprising the sequence of SEQ ID NO: 94, one heavy chain comprising an IL-10 variant positioned at the hinge region comprising the sequence of SEQ ID NO: 99, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antibody is an agonist antibody. In some embodiments, the antibody is an antagonist antibody.

In some embodiments, the antibody is an anti-PD-1 antibody. Thus in some embodiments, there is provided an IL-10 immunocytokine (“IL-10/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10 immunocytokine comprising: a) a full-length antibody specifically recognizing PD-1; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 111. In some embodiments, there is provided an IL-10/anti-PD-1 immunocytokine comprising: two light chains comprising the sequence of SEQ ID NO: 106, one heavy chain comprising an IL-10 variant positioned at the hinge region comprising the sequence of SEQ ID NO: 111, and one heavy chain comprises the sequence of SEQ ID NO: 107. In some embodiments, in the presence of binding of the antibody (or antigen-binding fragment) to PD-1, the activity (binding affinity to corresponding cytokine receptor or subunit thereof, and/or biological activity) of the IL-10 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-1. In some embodiments, in the absence of binding of the antibody (or antigen-binding fragment) to PD-1, the activity (binding affinity to corresponding cytokine receptor or subunit thereof, and/or biological activity) of the IL-10 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antibody is an agonist antibody. In some embodiments, the antibody is an antagonist antibody.

In some embodiments, the antibody is an anti-CD4 antibody. Thus in some embodiments, there is provided an IL-10 immunocytokine (“IL-10/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-CD4 immunocytokine comprising: a) a full-length antibody specifically recognizing CD4; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 82. In some embodiments, there is provided an IL-10/anti-CD4 immunocytokine comprising: two light chains comprising the sequence of SEQ ID NO: 77, one heavy chain comprising an IL-10 variant positioned at the hinge region comprising the sequence of SEQ ID NO: 82, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IL-10 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IL-10 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antibody is an agonist antibody. In some embodiments, the antibody is an antagonist antibody.

In some embodiments, the antigen-binding protein is an anti-CD8 antibody. Thus in some embodiments, there is provided an IL-10 immunocytokine (“IL-10/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-CD8 immunocytokine comprising: a) a full-length antibody specifically recognizing CD8; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD8 antibody comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 122. In some embodiments, there is provided an IL-10/anti-CD8 immunocytokine comprising: two light chains comprising the sequence of SEQ ID NO: 117, one heavy chain comprising an IL-10 variant positioned at the hinge region comprising the sequence of SEQ ID NO: 122, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IL-10 immunocytokine (“IL-10/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-CTLA-4 immunocytokine comprising: a) a full-length antibody specifically recognizing CTLA-4; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 134. In some embodiments, there is provided an IL-10/anti-CTLA-4 immunocytokine comprising: two light chains comprising the sequence of SEQ ID NO: 129, one heavy chain comprising an IL-10 variant positioned at the hinge region comprising the sequence of SEQ ID NO: 134, and one heavy chain comprises the sequence of SEQ ID NO: 130. In some embodiments, in the presence of binding of the antibody (or antigen-binding fragment) to CTLA-4, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IL-10 immunocytokine (“IL-10/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-10/anti-PD-L1 immunocytokine comprising: a) a full-length antibody specifically recognizing PD-L1; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 147. In some embodiments, there is provided an IL-10/anti-PD-L1 immunocytokine comprising: two light chains comprising the sequence of SEQ ID NO: 140, one heavy chain comprising an IL-10 variant positioned at the hinge region comprising the sequence of SEQ ID NO: 147, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IL-10 immunocytokine (“IL-10/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IL-10 or variant thereof (e.g., SEQ ID NO: 27 or 28), wherein the antibody comprises a heavy chain comprising a hinge region (e.g., any of SEQ ID NOs: 40-47, 50-52, and 55-59), and wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain, or between the C-terminus of CH1 and the N-terminus of the hinge region) of the heavy chain. In some embodiments, there is provided an IL-10/anti-CD25 immunocytokine comprising: a) a full-length antibody specifically recognizing CD25; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, one or both subunits of the IL-10 variant comprises the sequence of SEQ ID NO: 23. In some embodiments, the IL-10 variant comprises the sequence of SEQ ID NO: 28. In some embodiments, the heavy chain comprising the IL-10 variant positioned at the hinge region comprises the sequence of SEQ ID NO: 172. In some embodiments, there is provided an IL-10/anti-CD25 immunocytokine comprising: two light chains comprising the sequence of SEQ ID NO: 165, one heavy chain comprising an IL-10 variant positioned at the hinge region comprising the sequence of SEQ ID NO: 172, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IL-10 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IL-10 immunocytokine (“IL-10/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region (e.g., any of SEQ ID NOs: 40-47, 50-52, and 55-59), and an Fc domain subunit or portion thereof; and b) an IL-10 or variant thereof (e.g., SEQ ID NO: 27 or 28), wherein both subunits (e.g., any of SEQ ID NOs: 20-26) of the IL-10 or variant thereof are positioned in tandem (e.g., connected via an optional peptide linker such as any of SEQ ID NOs: 227-229) at the hinge region (e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, one or both subunits of the IL-10 variant comprises the sequence of SEQ ID NO: 23. In some embodiments, the IL-10 variant comprises the sequence of SEQ ID NO: 28. In some embodiments, the PD-L2-hinge-Fc fusion polypeptide comprising the IL-10 variant positioned at the hinge region comprises the sequence of SEQ ID NO: 184. In some embodiments, there is provided an IL-10/PD-L2-Fc immunocytokine comprising: a PD-L2-hinge-Fc fusion polypeptide comprising an IL-10 variant positioned at the hinge region comprises the sequence of SEQ ID NO: 184, and a PD-L2-hinge-Fc fusion polypeptide comprises the sequence of SEQ ID NO: 181. In some embodiments, in the presence of binding of the PD-L2 ligand to PD-1, the activity (binding affinity to corresponding cytokine receptor or subunit thereof, and/or biological activity) of the IL-10 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IL-10 cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-10 cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the cytokine or variant thereof is IL-12 or variant thereof. In some embodiments, there is provided an IL-12 immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IL-12 or variant thereof (e.g., SEQ ID NO: 35 or 36), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IL-12 immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IL-12 or variant thereof (e.g., SEQ ID NO: 35 or 36), wherein the antibody comprises a heavy chain comprising a hinge region, and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12 immunocytokine comprising: a) an antibody specifically recognizing a target antigen (e.g., CTLA-4, PD-L1, PD-L2, CD25, HER2, PD-1, CD3, CD4, CD123, or CD8); and b) an IL-12 or variant thereof (e.g., SEQ ID NO: 35 or 36), wherein the antibody comprises a heavy chain comprising from N-terminus to C-terminus: a VH domain, optionally a CH1 domain, [a first subunit of IL-12 or variant thereof (e.g., p40, such as any of SEQ ID NOs: 30-34), optionally a linker (e.g., peptide linker), a second subunit of IL-12 or variant thereof (e.g., p35, such as SEQ ID NO: 29)] at a hinge region, a CH2 domain, and optionally a CH3 domain. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant comprising one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30, wherein both p35 subunit (e.g., SEQ ID NO: 29) and p40 subunit of the IL-12 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant comprising one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30, wherein both p35 subunit (e.g., SEQ ID NO: 29) and p40 subunit of the IL-12 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant, wherein the p40 subunit of the IL-12 variant comprises the sequence of any of SEQ ID NOs: 31-34, wherein both p35 subunit (e.g., SEQ ID NO: 29) and p40 subunit of the IL-12 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant comprising an E59A/F60A mutation within the p40 subunit relative to a wildtype p40 subunit comprising the sequence of SEQ ID NO: 30, wherein both p35 subunit (e.g., SEQ ID NO: 29) and p40 subunit of the IL-12 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant, wherein the p40 subunit of the IL-12 variant comprises the sequence of SEQ ID NO: 31, and wherein both p35 subunit (e.g., SEQ ID NO: 29) and p40 subunit of the IL-12 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, the p40 subunit or variant thereof is at N-terminus of the p35 subunit or variant thereof (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, there is provided an IL-12 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-12 variant comprising the sequence of SEQ ID NO: 36, wherein the IL-12 variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IL-12 variant in a free state is no more than about 80% (such as no more than about any of 70%, 60%, 50%, 40%, 30%, 20%, 10%, or 5%) of that of a corresponding wildtype IL-12 in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IL-12 immunocytokine (“IL-12/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-HER2 immunocytokine comprising: a) a full-length antibody specifically recognizing HER2; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154.

In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit of the IL-12 variant comprises the sequence of SEQ ID NO: 31. In some embodiments, the p40 subunit and the p35 subunit of the IL-12 or variant thereof are connected by a linker. In some embodiments, the IL-12 variant comprises the sequence of SEQ ID NO: 36. In some embodiments, the heavy chain comprising the IL-12 variant positioned at the hinge region comprises the sequence of SEQ ID NO: 160. In some embodiments, there is provided an IL-12/anti-HER2 immunocytokine comprising: two light chains comprising the sequence of SEQ ID NO: 154, one heavy chain comprising an IL-12 variant positioned at the hinge region comprising the sequence of SEQ ID NO: 160, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IL-12 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IL-12 immunocytokine (“IL-12/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-CD3 immunocytokine comprising: a) a full-length antibody specifically recognizing CD3; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit of the IL-12 variant comprises the sequence of SEQ ID NO: 31. In some embodiments, the p40 subunit and the p35 subunit of the IL-12 or variant thereof are connected by a linker. In some embodiments, the IL-12 variant comprises the sequence of SEQ ID NO: 36. In some embodiments, the heavy chain comprising the IL-12 variant positioned at the hinge region comprises the sequence of SEQ ID NO: 100. In some embodiments, there is provided an IL-12/anti-CD3 immunocytokine comprising: two light chains comprising the sequence of SEQ ID NO: 94, one heavy chain comprising an IL-12 variant positioned at the hinge region comprising the sequence of SEQ ID NO: 100, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IL-12 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-PD-1 antibody. Thus in some embodiments, there is provided an IL-12 immunocytokine (“IL-12/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-PD-1 immunocytokine comprising: a) a full-length antibody specifically recognizing PD-1; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit of the IL-12 variant comprises the sequence of SEQ ID NO: 31. In some embodiments, the p40 subunit and the p35 subunit of the IL-12 or variant thereof are connected by a linker. In some embodiments, the IL-12 variant comprises the sequence of SEQ ID NO: 36. In some embodiments, the heavy chain comprising the IL-12 variant positioned at the hinge region comprises the sequence of SEQ ID NO: 112. In some embodiments, there is provided an IL-12/anti-PD-1 immunocytokine comprising: two light chains comprising the sequence of SEQ ID NO: 106, one heavy chain comprising an IL-12 variant positioned at the hinge region comprising the sequence of SEQ ID NO: 112, and one heavy chain comprises the sequence of SEQ ID NO: 107. In some embodiments, in the presence of binding of the antibody (or antigen-binding fragment) to PD-1, the activity (binding affinity to corresponding cytokine receptor or subunit thereof, and/or biological activity) of the IL-12 or variant thereof increases at least about 20% (such as at least about any of 30%, 40%, 0%, 60%, 70%, 80%, 90%, 100%, 200%, 300%, 400%, 500%, or more) compared to that in the absence of binding of the antibody (or antigen-binding fragment) to PD-1. In some embodiments, in the absence of binding of the antibody (or antigen-binding fragment) to PD-1, the activity (binding affinity to corresponding cytokine receptor or subunit thereof, and/or biological activity) of the IL-12 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, there is provided an IL-12/IL-2 immunocytokine (“IL-12/IL-2/anti-PD-1 immunocytokine”) comprising: a) an antigen-binding protein (e.g., full-length antibody, heavy chain only antibody, or antigen-binding fragment fused to an Fc domain subunit or portion thereof) specifically recognizing PD-1; b) an IL-12 or variant thereof (e.g., SEQ ID NO: 35 or 36); and c) an IL-2 or variant thereof (e.g., SEQ ID NO: 1 or 2), wherein the antigen-binding protein comprises two antigen-binding polypeptides (e.g., antibody heavy chain, or antigen-binding fragment-Fc fusion polypeptide) each comprising from N′ to C′: an antigen-binding fragment (e.g., VHH, scFv, or VH), a hinge region (e.g., any of SEQ ID NOs: 40-47, 50-52, and 55-59), and an Fc domain subunit or portion thereof (e.g., CH2+CH3, or CH2 only), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the first antigen-binding polypeptide (e.g., antibody heavy chain), and wherein the IL-2 or variant thereof is positioned at the hinge region (e.g., within the hinge region, or between the C-terminus of CH1 and the N-terminus of the hinge region) of the second antigen-binding polypeptide (e.g., antibody heavy chain). In some embodiments, there is provided an IL-12/IL-2/anti-PD-1 immunocytokine comprising: a) a full-length antibody specifically recognizing PD-1 comprising two heavy chains; b) an IL-12 or variant thereof (e.g., SEQ ID NO: 35 or 36); and c) an IL-2 or variant thereof (e.g., SEQ ID NO: 1 or 2); wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of the first heavy chain of the full-length antibody; wherein the IL-2 or variant thereof (e.g., SEQ ID NO: 1 or 2) is positioned at the hinge region (e.g., any of SEQ ID NOs: 40-47, 50-52, and 55-59)(e.g., within the hinge region, or between the C-terminus of CH1 and the N-terminus of the hinge region) of the second heavy chain of the full-length antibody; and wherein the anti-PD-1 antibody comprises VH-CDR1, VH-CDR2, and VH-CDR3 of a V_{H} comprising the sequence of SEQ ID NO: 102, and VL-CDR1, VL-CDR2, and VL-CDR3 of a V_{L} comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit of the IL-12 variant comprises the sequence of SEQ ID NO: 31. In some embodiments, the p40 subunit and the p35 subunit of the IL-12 or variant thereof are connected by a linker. In some embodiments, the IL-12 variant comprises the sequence of SEQ ID NO: 36.

In some embodiments, the IL-2 variant comprises the sequence of SEQ ID NO: 2. In some embodiments, the heavy chain comprising the IL-12 variant positioned at the hinge region comprises the sequence of SEQ ID NO: 112. In some embodiments, the heavy chain comprising the IL-2 variant positioned at the hinge region comprises the sequence of SEQ ID NO: 108. In some embodiments, in the presence of binding of the antigen-binding protein (e.g., antibody) or antigen-binding fragment to PD-1 (e.g., binding of the first antigen-binding fragment to PD-1, and/or binding of the second antigen-binding fragment to PD-1), the activity (binding affinity to corresponding cytokine receptor or subunit thereof, and/or biological activity) of the IL-12 and/or IL-2 (or variant thereof) increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-1 (e.g., neither antigen-binding fragment binds to PD-1). In some embodiments, in the absence of binding of the antigen-binding protein (e.g., antibody) or antigen-binding fragment to PD-1 (e.g., neither antigen-binding fragment binds to PD-1), the activity (binding affinity to corresponding cytokine receptor or subunit thereof, and/or biological activity) of the IL-12 and/or IL-2 (or variant thereof) positioned at the hinge region of each antigen-binding polypeptide (e.g., antibody heavy chain, or antigen-binding fragment-Fc polypeptide) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 and/or IL-2 (or variant thereof) in a free state. The parental antigen-binding protein (e.g., antibody) can be monospecific or multispecific (e.g., bispecific). The parental antigen-binding protein (e.g., antibody) can be homodimeric or heterodimeric. The parental antigen-binding protein (e.g., antibody) can be an agonist or an antagonist.

In some embodiments, the antibody is an anti-CD4 antibody. Thus in some embodiments, there is provided an IL-12 immunocytokine (“IL-12/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-CD4 immunocytokine comprising: a) a full-length antibody specifically recognizing CD4; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, the p40 subunit of the IL-12 variant comprises the sequence of SEQ ID NO: 31. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the IL-12 variant comprises the sequence of SEQ ID NO: 36. In some embodiments, the heavy chain comprising the IL-12 variant positioned at the hinge region comprises the sequence of SEQ ID NO: 83. In some embodiments, there is provided an IL-12/anti-CD4 immunocytokine comprising: two light chains comprising the sequence of SEQ ID NO: 77, one heavy chain comprising an IL-12 variant positioned at the hinge region comprising the sequence of SEQ ID NO: 83, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IL-12 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IL-12 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IL-12 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-CD8 antibody. Thus in some embodiments, there is provided an IL-12 immunocytokine (“IL-12/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-CD8 immunocytokine comprising: a) a full-length antibody specifically recognizing CD8; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD8 antibody comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, the p40 subunit of the IL-12 variant comprises the sequence of SEQ ID NO: 31. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the IL-12 variant comprises the sequence of SEQ ID NO: 36. In some embodiments, the heavy chain comprising the IL-12 variant positioned at the hinge region comprises the sequence of SEQ ID NO: 123. In some embodiments, there is provided an IL-12/anti-CD8 immunocytokine comprising: two light chains comprising the sequence of SEQ ID NO: 117, one heavy chain comprising an IL-12 variant positioned at the hinge region comprising the sequence of SEQ ID NO: 123, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IL-12 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IL-12 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IL-12 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IL-12 immunocytokine (“IL-12/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-CTLA-4 immunocytokine comprising: a) a full-length antibody specifically recognizing CTLA-4; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the p40 subunit of the IL-12 variant comprises the sequence of SEQ ID NO: 31. In some embodiments, the IL-12 variant comprises the sequence of SEQ ID NO: 36. In some embodiments, the heavy chain comprising the IL-12 variant positioned at the hinge region comprises the sequence of SEQ ID NO: 135. In some embodiments, there is provided an IL-12/anti-CTLA-4 immunocytokine comprising: two light chains comprising the sequence of SEQ ID NO: 129, one heavy chain comprising an IL-12 variant positioned at the hinge region comprising the sequence of SEQ ID NO: 135, and one heavy chain comprises the sequence of SEQ ID NO: 130. In some embodiments, in the presence of binding of the antibody (or antigen-binding fragment) to CTLA-4, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IL-12 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IL-12 immunocytokine (“IL-12/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-12/anti-PD-L1 immunocytokine comprising: a) a full-length antibody specifically recognizing PD-L1; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the p40 subunit of the IL-12 variant comprises the sequence of SEQ ID NO: 31. In some embodiments, the IL-12 variant comprises the sequence of SEQ ID NO: 36. In some embodiments, the heavy chain comprising the IL-12 variant positioned at the hinge region comprises the sequence of SEQ ID NO: 148. In some embodiments, there is provided an IL-12/anti-PD-L1 immunocytokine comprising: two light chains comprising the sequence of SEQ ID NO: 140, one heavy chain comprising an IL-12 variant positioned at the hinge region comprising the sequence of SEQ ID NO: 148, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IL-12 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IL-12 immunocytokine (“IL-12/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IL-12 or variant thereof (e.g., SEQ ID NO: 35 or 36), wherein the antibody comprises a heavy chain comprising a hinge region (e.g., any of SEQ ID NOs: 40-47, 50-52, and 55-59), and wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain, or between the C-terminus of CH1 and the N-terminus of the hinge region) of the heavy chain. In some embodiments, there is provided an IL-12/anti-CD25 immunocytokine comprising: a) a full-length antibody specifically recognizing CD25; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the p40 subunit of the IL-12 variant comprises the sequence of SEQ ID NO: 31. In some embodiments, the IL-12 variant comprises the sequence of SEQ ID NO: 36. In some embodiments, the heavy chain comprising the IL-12 variant positioned at the hinge region comprises the sequence of SEQ ID NO: 173. In some embodiments, there is provided an IL-12/anti-CD25 immunocytokine comprising: two light chains comprising the sequence of SEQ ID NO: 165, one heavy chain comprising an IL-12 variant positioned at the hinge region comprising the sequence of SEQ ID NO: 173, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IL-12 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IL-12 immunocytokine (“IL-12/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region (e.g., any of SEQ ID NOs: 40-47, 50-52, and 55-59), and an Fc domain subunit or portion thereof; and b) an IL-12 or variant thereof (e.g., SEQ ID NO: 35 or 36), wherein both p35 subunit (e.g., SEQ ID NO: 29) and p40 subunit (e.g., any of SEQ ID NOs: 30-34) of the IL-12 or variant thereof are positioned in tandem (e.g., connected via an optional peptide linker such as any of SEQ ID NOs: 227-229) at the hinge region (e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, the p40 subunit or variant thereof (e.g., any of SEQ ID NOs: 30-34) is at N-terminus of the p35 subunit or variant thereof (e.g., SEQ ID NO: 29) (e.g., p40-optional linker (e.g., any of SEQ ID NOs: 227-229)-p35). In some embodiments, the p35 subunit or variant thereof is at N-terminus of the p40 subunit or variant thereof (e.g., p35-optional linker (e.g., any of SEQ ID NOs: 227-229)-p40). In some embodiments, the p40 subunit and the p35 subunit of the IL-12 or variant thereof are connected by a linker. In some embodiments, the p40 subunit of the IL-12 variant comprises the sequence of SEQ ID NO: 31. In some embodiments, the IL-12 variant comprises the sequence of SEQ ID NO: 36. In some embodiments, the PD-L2-hinge-Fc fusion polypeptide comprising the IL-12 variant positioned at the hinge region comprises the sequence of SEQ ID NO: 186. In some embodiments, there is provided an IL-12/PD-L2-Fc immunocytokine comprising: a PD-L2-hinge-Fc fusion polypeptide comprising an IL-12 variant positioned at the hinge region comprises the sequence of SEQ ID NO: 186, and a PD-L2-hinge-Fc fusion polypeptide comprises the sequence of SEQ ID NO: 185. In some embodiments, the p40 subunit of the IL-12 variant comprises the sequence of SEQ ID NO: 33. In some embodiments, the IL-12 variant comprises the sequence of SEQ ID NO: 275. In some embodiments, the PD-L2-hinge-Fc fusion polypeptide comprising the IL-12 variant positioned at the hinge region comprises the sequence of SEQ ID NO: 274. In some embodiments, there is provided an IL-12/PD-L2-Fc immunocytokine comprising: a PD-L2-hinge-Fc fusion polypeptide comprising an IL-12 variant positioned at the hinge region comprises the sequence of SEQ ID NO: 274, and a PD-L2-hinge-Fc fusion polypeptide comprises the sequence of SEQ ID NO: 185. In some embodiments, in the presence of binding of the PD-L2 ligand to PD-1, the activity (binding affinity to corresponding cytokine receptor or subunit thereof, and/or biological activity) of the IL-12 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IL-12 cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-12 cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the cytokine or variant thereof is IL-23 or variant thereof. In some embodiments, there is provided an IL-23 immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) an IL-23 or variant thereof (e.g., SEQ ID NO: 38 or 39), wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2 extracellular domain, receptor, VHH, scFv, or VH), a hinge region, and an Fc domain subunit or portion thereof (e.g., CH2+CH3, or CH2 only), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at (e.g., at the N′ of, at the C′ of, or within) the hinge region. In some embodiments, the antigen-binding protein is an antibody (e.g., full-length antibody) specifically recognizing the target antigen, the antigen-binding polypeptide comprising the hinge region is a heavy chain of the antibody, and both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region of the heavy chain. In some embodiments, the antigen-binding fragment is a ligand, and the target antigen is a receptor specifically recognized by the ligand. In some embodiments, the antigen-binding fragment is a receptor, and the target antigen is a ligand specifically recognized by the receptor. In some embodiments, there is provided an IL-23 immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD3, CD4, CD123, or CD8); and b) an IL-23 or variant thereof (e.g., SEQ ID NO: 38 or 39), wherein the antibody comprises a heavy chain comprising a hinge region, and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23 immunocytokine comprising: a) an antibody specifically recognizing a target antigen (e.g., CTLA-4, PD-L1, PD-L2, CD25, HER2, PD-1, CD3, CD4, CD123, or CD8); and b) an IL-23 or variant thereof (e.g., SEQ ID NO: 38 or 39), wherein the antibody comprises a heavy chain comprising from N-terminus to C-terminus: a VH domain, optionally a CH1 domain, [a first subunit of IL-23 or variant thereof (e.g., p40, such as any of SEQ ID NOs: 30-34), optionally a linker (e.g., peptide linker), a second subunit of IL-23 or variant thereof (e.g., p19, such as SEQ ID NO: 37)] at a hinge region, a CH2 domain, and optionally a CH3 domain. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., between the C-terminus of CH1 and the N-terminus of the hinge region) of a heavy chain of the full-length antibody. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant comprising one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30, wherein both p19 subunit (e.g., SEQ ID NO: 37) and p40 subunit of the IL-23 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant comprising one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30, wherein both p19 subunit (e.g., SEQ ID NO: 37) and p40 subunit of the IL-23 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant, wherein the p40 subunit of the IL-23 variant comprises the sequence of any of SEQ ID NOs: 31-34, wherein both p19 subunit (e.g., SEQ ID NO: 37) and p40 subunit of the IL-23 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant comprising an E59A/F60A mutation within the p40 subunit relative to a wildtype p40 subunit comprising the sequence of SEQ ID NO: 30, wherein both p19 subunit (e.g., SEQ ID NO: 37) and p40 subunit of the IL-23 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant, wherein the p40 subunit of the IL-23 variant comprises the sequence of SEQ ID NO: 31, and wherein both p19 subunit (e.g., SEQ ID NO: 37) and p40 subunit of the IL-23 variant are positioned in tandem (e.g., connected via an optional peptide linker such as any of SEQ ID NOs: 227-229) at the hinge region (e.g., within the hinge region, or between the C-terminus of CH1 and the N-terminus of the hinge region) of a heavy chain of the full-length antibody. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit and the p19 subunit of the IL-23 or variant thereof are connected by a linker. In some embodiments, there is provided an IL-23 immunocytokine comprising: a) a full-length antibody specifically recognizing a target antigen (e.g., CTLA-4, PD-L1, PD-L2, CD25, HER2, PD-1, CD3, CD4, CD123, or CD8); and b) an IL-23 variant comprising the sequence of SEQ ID NO: 39, wherein the IL-23 variant is positioned at the hinge region (e.g., within the hinge region, or between the C-terminus of CH1 and the N-terminus of the hinge region) of a heavy chain of the full-length antibody. In some embodiments, the activity (binding affinity to corresponding cytokine receptor or subunit thereof, and/or biological activity) of the IL-23 variant in a free state is no more than about 80% (such as no more than about any of 70%, 60%, 50%, 40%, 30%, 20%, 10%, or 5%) of that of a corresponding wildtype IL-23 in a free state. In some embodiments, the antigen-binding protein (e.g., antibody or fragment thereof, or antigen-binding fragment-hinge-Fc fusion protein such as ligand/receptor-hinge-Fc fusion protein) is monospecific. In some embodiments, the antigen-binding protein is multispecific (e.g., bispecific). In some embodiments, the parental antigen-binding protein (e.g., antibody such as full-length antibody, or antigen-binding fragment-hinge-Fc fusion protein such as ligand/receptor-hinge-Fc fusion protein) is homodimeric. In some embodiments, the parental antigen-binding protein is heterodimeric. In some embodiments, the antigen-binding protein is an agonist. In some embodiments, the antigen-binding protein is an antagonist. In some embodiments, the hinge region comprises the sequence of any of SEQ ID NOs: 40-47, 50-52, and 55-59.

In some embodiments, the antigen-binding protein is an anti-HER2 antibody. Thus in some embodiments, there is provided an IL-23 immunocytokine (“IL-23/anti-HER2 immunocytokine”) comprising: a) an antibody (e.g., full-length antibody, heavy chain only antibody, or antigen-binding fragment fused to an Fc domain subunit or portion thereof) specifically recognizing HER2; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-HER2 immunocytokine comprising: a) a full-length antibody specifically recognizing HER2; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-HER2 antibody comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the parental anti-HER2 antibody comprises two heavy chains each comprising the sequence of SEQ ID NO: 152 or 153, and two light chains each comprising the sequence of SEQ ID NO: 154.

In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 161. In some embodiments, there is provided an IL-23/anti-HER2 immunocytokine comprising: two light chains comprising the sequence of SEQ ID NO: 154, one heavy chain comprising an IL-23 variant positioned at the hinge region comprising the sequence of SEQ ID NO: 161, and one heavy chain comprises the sequence of SEQ ID NO: 155. In some embodiments, in the presence of binding of the antibody (or antigen-binding fragment) to HER2, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to HER2. In some embodiments, in the absence of binding of the antibody (or antigen-binding fragment) to HER2, the activity (binding affinity to corresponding cytokine receptor or subunit thereof, and/or biological activity) of the IL-23 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CD3 antibody. Thus in some embodiments, there is provided an IL-23 immunocytokine (“IL-23/anti-CD3 immunocytokine”) comprising: a) an antibody (e.g., full-length antibody, heavy chain only antibody, or antigen-binding fragment fused to an Fc domain subunit or portion thereof) specifically recognizing CD3; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-CD3 immunocytokine comprising: a) a full-length antibody specifically recognizing CD3; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD3 antibody comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 antibody comprises a VH comprising the sequence of SEQ ID NO: 91 and a VL comprising the sequence of SEQ ID NO: 92. In some embodiments, the parental anti-CD3 antibody comprises two heavy chains each comprising the sequence of SEQ ID NO: 93, and two light chains each comprising the sequence of SEQ ID NO: 94. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 101. In some embodiments, there is provided an IL-23/anti-CD3 immunocytokine comprising: two light chains comprising the sequence of SEQ ID NO: 94, one heavy chain comprising an IL-23 variant positioned at the hinge region comprising the sequence of SEQ ID NO: 101, and one heavy chain comprises the sequence of SEQ ID NO: 95. In some embodiments, in the presence of binding of the antibody (or antigen-binding fragment) to CD3, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD3. In some embodiments, in the absence of binding of the antibody (or antigen-binding fragment) to CD3, the activity (binding affinity to corresponding cytokine receptor or subunit thereof, and/or biological activity) of the IL-23 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-PD-1 antibody. Thus in some embodiments, there is provided an IL-23 immunocytokine (“IL-23/anti-PD-1 immunocytokine”) comprising: a) an antibody (e.g., full-length antibody, heavy chain only antibody, or antigen-binding fragment fused to an Fc domain subunit or portion thereof) specifically recognizing PD-1; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-PD-1 immunocytokine comprising: a) a full-length antibody specifically recognizing PD-1; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-1 antibody comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody comprises a VH comprising the sequence of SEQ ID NO: 102 and a VL comprising the sequence of SEQ ID NO: 103. In some embodiments, the parental anti-PD-1 antibody comprises two heavy chains each comprising the sequence of SEQ ID NO: 104 or 105, and two light chains each comprising the sequence of SEQ ID NO: 106. In some embodiments, the p40 subunit of the IL-23 variant comprises the sequence of SEQ ID NO: 31. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 113. In some embodiments, there is provided an IL-23/anti-PD-1 immunocytokine comprising: two light chains comprising the sequence of SEQ ID NO: 106, one heavy chain comprising an IL-23 variant positioned at the hinge region comprising the sequence of SEQ ID NO: 113, and one heavy chain comprises the sequence of SEQ ID NO: 107. In some embodiments, in the presence of binding of the antibody (or antigen-binding fragment) to PD-1, the activity (binding affinity to corresponding cytokine receptor or subunit thereof, and/or biological activity) of the IL-23 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-1. In some embodiments, in the absence of binding of the antibody (or antigen-binding fragment) to PD-1, the activity (binding affinity to corresponding cytokine receptor or subunit thereof, and/or biological activity) of the IL-23 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-CD4 antibody. Thus in some embodiments, there is provided an IL-23 immunocytokine (“IL-23/anti-CD4 immunocytokine”) comprising: a) an antibody (e.g., full-length antibody, heavy chain only antibody, or antigen-binding fragment fused to an Fc domain subunit or portion thereof) specifically recognizing CD4; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-CD4 immunocytokine comprising: a) a full-length antibody specifically recognizing CD4; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD4 antibody comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody comprises a VH comprising the sequence of SEQ ID NO: 73 and a VL comprising the sequence of SEQ ID NO: 74. In some embodiments, the parental anti-CD4 antibody comprises two heavy chains each comprising the sequence of SEQ ID NO: 75 or 76, and two light chains each comprising the sequence of SEQ ID NO: 77. In some embodiments, the p40 subunit of the IL-23 variant comprises the sequence of SEQ ID NO: 31. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 84. In some embodiments, there is provided an IL-23/anti-CD4 immunocytokine comprising: two light chains comprising the sequence of SEQ ID NO: 77, one heavy chain comprising an IL-23 variant positioned at the hinge region comprising the sequence of SEQ ID NO: 84, and one heavy chain comprises the sequence of SEQ ID NO: 78. In some embodiments, in the presence of binding of the antibody (or antigen-binding fragment) to CD4, the activity (binding affinity to corresponding cytokine receptor or subunit thereof, and/or biological activity) of the IL-23 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD4. In some embodiments, in the absence of binding of the antibody (or antigen-binding fragment) to CD4, the activity (binding affinity to corresponding cytokine receptor or subunit thereof, and/or biological activity) of the IL-23 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IL-23 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antibody is an anti-CD8 antibody. Thus in some embodiments, there is provided an IL-23 immunocytokine (“IL-23/anti-CD8 immunocytokine”) comprising: a) an antibody (e.g., full-length antibody, heavy chain only antibody, or antigen-binding fragment fused to an Fc domain subunit or portion thereof) specifically recognizing CD8; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-CD8 immunocytokine comprising: a) a full-length antibody specifically recognizing CD8; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD8 antibody comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody comprises a VH comprising the sequence of SEQ ID NO: 114 and a VL comprising the sequence of SEQ ID NO: 115. In some embodiments, the parental anti-CD8 antibody comprises two heavy chains each comprising the sequence of SEQ ID NO: 116, and two light chains each comprising the sequence of SEQ ID NO: 117. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 124. In some embodiments, there is provided an IL-23/anti-CD8 immunocytokine comprising: two light chains comprising the sequence of SEQ ID NO: 117, one heavy chain comprising an IL-23 variant positioned at the hinge region comprising the sequence of SEQ ID NO: 124, and one heavy chain comprises the sequence of SEQ ID NO: 118. In some embodiments, in the presence of binding of the antibody (or antigen-binding fragment) to CD8, the activity (binding affinity to corresponding cytokine receptor or subunit thereof, and/or biological activity) of the IL-23 or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD8. In some embodiments, in the absence of binding of the antibody (or antigen-binding fragment) to CD8, the activity (binding affinity to corresponding cytokine receptor or subunit thereof, and/or biological activity) of the IL-23 or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding IL-23 or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-CTLA-4 antibody. Thus in some embodiments, there is provided an IL-23 immunocytokine (“IL-23/anti-CTLA-4 immunocytokine”) comprising: a) an antibody (e.g., full-length antibody, heavy chain only antibody, or antigen-binding fragment fused to an Fc domain subunit or portion thereof) specifically recognizing CTLA-4; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-CTLA-4 immunocytokine comprising: a) a full-length antibody specifically recognizing CTLA-4; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CTLA-4 antibody comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody comprises a VH comprising the sequence of SEQ ID NO: 125 and a VL comprising the sequence of SEQ ID NO: 126. In some embodiments, the parental anti-CTLA-4 antibody comprises two heavy chains each comprising the sequence of SEQ ID NO: 127 or 128, and two light chains each comprising the sequence of SEQ ID NO: 129. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 124. In some embodiments, the heavy chain comprising the IL-23 variant positioned at the hinge region comprises the sequence of SEQ ID NO: 136. In some embodiments, there is provided an IL-23/anti-CTLA-4 immunocytokine comprising: two light chains comprising the sequence of SEQ ID NO: 129, one heavy chain comprising an IL-23 variant positioned at the hinge region comprising the sequence of SEQ ID NO: 136, and one heavy chain comprises the sequence of SEQ ID NO: 130. In some embodiments, in the presence of binding of the antibody (or antigen-binding fragment) to CTLA-4, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CTLA-4. In some embodiments, in the absence of binding of the antibody (or antigen-binding fragment) to CTLA-4, the activity (binding affinity to corresponding cytokine receptor or subunit thereof, and/or biological activity) of the IL-23 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is an anti-PD-L1 antibody. Thus in some embodiments, there is provided an IL-23 immunocytokine (“IL-23/anti-PD-L1 immunocytokine”) comprising: a) an antibody (e.g., full-length antibody, heavy chain only antibody, or antigen-binding fragment fused to an Fc domain subunit or portion thereof) specifically recognizing PD-L1; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain. In some embodiments, there is provided an IL-23/anti-PD-L1 immunocytokine comprising: a) a full-length antibody specifically recognizing PD-L1; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-PD-L1 antibody comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody comprises a VH comprising the sequence of SEQ ID NO: 137 and a VL comprising the sequence of SEQ ID NO: 138. In some embodiments, the parental anti-PD-L1 antibody comprises two heavy chains each comprising the sequence of SEQ ID NO: 139, and two light chains each comprising the sequence of SEQ ID NO: 140. In some embodiments, the parental anti-PD-L1 antibody comprises two heavy chains each comprising the sequence of SEQ ID NO: 141, and two light chains each comprising the sequence of SEQ ID NO: 142. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 149. In some embodiments, there is provided an IL-23/anti-PD-L1 immunocytokine comprising: two light chains comprising the sequence of SEQ ID NO: 140, one heavy chain comprising an IL-23 variant positioned at the hinge region comprising the sequence of SEQ ID NO: 149, and one heavy chain comprises the sequence of SEQ ID NO: 143. In some embodiments, in the presence of binding of the antibody (or antigen-binding fragment) to PD-L1, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to PD-L1. In some embodiments, in the absence of binding of the antibody (or antigen-binding fragment) to PD-L1, the activity (binding affinity to corresponding cytokine receptor or subunit thereof, and/or biological activity) of the IL-23 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric.

In some embodiments, the antigen-binding protein is an anti-CD25 antibody. Thus in some embodiments, there is provided an IL-23 immunocytokine (“IL-23/anti-CD25 immunocytokine”) comprising: a) an antibody (e.g., full-length antibody, heavy chain only antibody, or antigen-binding fragment fused to an Fc domain subunit or portion thereof) specifically recognizing CD25; and b) an IL-23 or variant thereof (e.g., SEQ ID NO: 38 or 39), wherein the antibody comprises a heavy chain comprising a hinge region (e.g., any of SEQ ID NOs: 40-47, 50-52, and 55-59), and wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of CH1 and the N-terminus of the hinge region) of the heavy chain, or between the C-terminus of CH1 and the N-terminus of the hinge region) of the heavy chain. In some embodiments, there is provided an IL-23/anti-CD25 immunocytokine comprising: a) a full-length antibody specifically recognizing CD25; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., any of SEQ ID NOs: 40-47, 50-52, and 55-59)(e.g., within the hinge region, or between the C-terminus of CH1 and the N-terminus of the hinge region) of a heavy chain of the full-length antibody; and wherein the anti-CD25 antibody comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody comprises a VH comprising the sequence of SEQ ID NO: 162 and a VL comprising the sequence of SEQ ID NO: 163. In some embodiments, the parental anti-CD25 antibody comprises two heavy chains each comprising the sequence of SEQ ID NO: 164, and two light chains each comprising the sequence of SEQ ID NO: 165. In some embodiments, the parental anti-CD25 antibody comprises two heavy chains each comprising the sequence of SEQ ID NO: 166, and two light chains each comprising the sequence of SEQ ID NO: 167. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the heavy chain comprising the IL-23 variant positioned at the hinge region comprises the sequence of SEQ ID NO: 174. In some embodiments, there is provided an IL-23/anti-CD25 immunocytokine comprising: two light chains comprising the sequence of SEQ ID NO: 165, one heavy chain comprising an IL-23 variant positioned at the hinge region comprising the sequence of SEQ ID NO: 174, and one heavy chain comprises the sequence of SEQ ID NO: 168. In some embodiments, in the presence of binding of the antibody (or antigen-binding fragment) to CD25, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antibody (or antigen-binding fragment) to CD25. In some embodiments, in the absence of binding of the antibody (or antigen-binding fragment) to CD25, the activity (binding affinity to corresponding cytokine receptor or subunit thereof, and/or biological activity) of the IL-23 cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the antibody is monospecific. In some embodiments, the antibody is multispecific (e.g., bispecific). In some embodiments, the antibody is homodimeric. In some embodiments, the antibody is heterodimeric. In some embodiments, the antibody is an agonist antibody. In some embodiments, the antibody is an antagonist antibody.

In some embodiments, the antigen-binding protein is a PD-L2 (e.g., PD-L2 extracellular domain)-hinge-Fc fusion protein. Thus in some embodiments, there is provided an IL-23 immunocytokine (“IL-23/PD-L2-Fc immunocytokine”) comprising: a) a PD-L2-hinge-Fc fusion polypeptide comprising from N′ to C′: a PD-L2 (e.g., SEQ ID NO: 176) specifically recognizing PD-1, a hinge region (e.g., any of SEQ ID NOs: 40-47, 50-52, and 55-59), and an Fc domain subunit or portion thereof; and b) an IL-23 or variant thereof (e.g., SEQ ID NO: 38 or 39), wherein both p19 subunit (e.g., SEQ ID NO: 37) and p40 subunit (e.g., any of SEQ ID NOs: 30-34) of the IL-23 or variant thereof are positioned in tandem (e.g., connected via an optional peptide linker such as any of SEQ ID NOs: 227-229) at the hinge region (e.g., within the hinge region, or between the C-terminus of PD-L2 and the N-terminus of the hinge region) of the PD-L2-hinge-Fc fusion polypeptide. In some embodiments, the parental PD-L2-hinge-Fc fusion protein comprises two PD-L2-hinge-Fc fusion polypeptides each comprising the sequence of SEQ ID NO: 177. In some embodiments, the p40 subunit or variant thereof (e.g., any of SEQ ID NOs: 30-34) is at N-terminus of the p19 subunit or variant thereof (e.g., SEQ ID NO: 37) (e.g., p40-optional linker (e.g., any of SEQ ID NOs: 227-229)-p19). In some embodiments, the p19 subunit or variant thereof is at N-terminus of the p40 subunit or variant thereof (e.g., p19-optional linker (e.g., any of SEQ ID NOs: 227-229)-p40). In some embodiments, the p40 subunit of the IL-23 variant comprises the sequence of SEQ ID NO: 31. In some embodiments, the p40 subunit and the p19 subunit of the IL-23 or variant thereof are connected by a linker. In some embodiments, the IL-23 variant comprises the sequence of SEQ ID NO: 39. In some embodiments, the PD-L2-hinge-Fc fusion polypeptide comprising the IL-23 variant positioned at the hinge region comprises the sequence of SEQ ID NO: 187. In some embodiments, there is provided an IL-23/PD-L2-Fc immunocytokine comprising: a PD-L2-hinge-Fc fusion polypeptide comprising an IL-23 variant positioned at the hinge region comprises the sequence of SEQ ID NO: 187, and a PD-L2-hinge-Fc fusion polypeptide comprises the sequence of SEQ ID NO: 185. In some embodiments, in the presence of binding of the PD-L2 ligand to PD-1, the activity (binding affinity to corresponding cytokine receptor or subunit thereof, and/or biological activity) of the IL-23 cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the PD-L2 ligand to PD-1. In some embodiments, in the absence of binding of the PD-L2 ligand to PD-1, the activity (binding affinity to corresponding cytokine receptor or subunit thereof, and/or biological activity) of the IL-23 cytokine or variant thereof positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding IL-23 cytokine or variant thereof in a free state. In some embodiments, the PD-L2-hinge-Fc fusion protein is monospecific. In some embodiments, the PD-L2-hinge-Fc fusion protein is multispecific (e.g., bispecific). In some embodiments, the PD-L2-hinge-Fc fusion protein is homodimeric. In some embodiments, the PD-L2-hinge-Fc fusion protein is heterodimeric. In some embodiments, the PD-L2 moiety is an agonist that can stimulate or enhance PD-1 signaling. In some embodiments, the PD-L2 moiety is an antagonist that can reduce or block PD-1 signaling.

In some embodiments, the immunocytokine described herein does not comprise a full-length parental antibody (hereinafter also referred to as “partial antibody immunocytokine” or “partial Fc-fusion immunocytokine”). See, e.g., light grey portions of FIGS. 3A-3C. In some embodiments, the present invention also provides an immunocytokine comprising: (a) a first antigen-binding polypeptide comprising from N-terminus to C-terminus: a first scFv (V_{H}-V_{L}, or VL-V_{H} configuration) specifically recognizing a first target antigen (e.g., CTLA-4, PD-L1, PD-L2, CD25, HER2, PD-1, CD3, CD4, CD123, or CD8), a cytokine or variant thereof (e.g., IL-2, IFN-α (e.g., IFN-α2b), IFN-γ, IL-10, IL-12, or IL-23) at a hinge region or portion thereof (e.g., within the hinge region, or between the C′ of the first scFv and the N′ of the hinge region), and a first subunit of an Fc domain (e.g., IgG1, IgG4, or effectorless IgG1) or portion thereof (e.g., CH2); and (b) a second antigen-binding polypeptide comprising from N-terminus to C-terminus: a second scFv (VH-VL, or VL-VH configuration) specifically recognizing a second target antigen (e.g., CTLA-4, PD-L1, PD-L2, CD25, HER2, PD-1, CD3, CD4, CD123, or CD8), a hinge region or portion thereof (e.g., any of SEQ ID NOs: 40-47, 50-52, and 55-59), and a second subunit of the Fc domain or portion thereof. See, e.g., FIG. 3A. In some embodiments, there is provided an immunocytokine comprising: (a) a first antigen-binding polypeptide comprising from N-terminus to C-terminus: an scFv (VH-VL, or VL-VH configuration) specifically recognizing a first target antigen (e.g., CTLA-4, PD-L1, PD-L2, CD25, HER2, PD-1, CD3, CD4, CD123, or CD8), a cytokine or variant thereof (e.g., IL-2, IFN-α (e.g., IFN-α2b), IFN-γ, IL-10, IL-12, or IL-23) at a hinge region or portion thereof (e.g., within the hinge region, or between the C′ of the scFv and the N′ of the hinge region), and a first subunit of an Fc domain (e.g., IgG1, IgG4, or effectorless IgG1) or portion thereof (e.g., CH2); (b) a second antigen-binding polypeptide comprising from N-terminus to C-terminus: a VH, a CH1, a hinge region or portion thereof (e.g., any of SEQ ID NOs: 40-47, 50-52, and 55-59), and a second subunit of the Fc domain or portion thereof; and (c) a third antigen-binding polypeptide comprising from N-terminus to C-terminus: a VL, and a CL; wherein the VH and CH1 of the second antigen-binding polypeptide and the VL and CL of the third antigen-binding polypeptide form a Fab specifically recognizing a second target antigen (e.g., CTLA-4, PD-L1, PD-L2, CD25, HER2, PD-1, CD3, CD4, CD123, or CD8). See, e.g., FIG. 3B. In some embodiments, there is provided an immunocytokine comprising: (a) a first antigen-binding polypeptide comprising from N-terminus to C-terminus: a VH, a CH1, a cytokine or variant thereof (e.g., IL-2, IFN-α (e.g., IFN-α2b), IFN-γ, IL-10, IL-12, or IL-23) at a hinge region or portion thereof (e.g., any of SEQ ID NOs: 40-47, 50-52, and 55-59), and a first subunit of an Fc domain (e.g., IgG1, IgG4, or effectorless IgG1) or portion thereof (e.g., CH2); (b) a second antigen-binding polypeptide comprising from N-terminus to C-terminus: a VL, and a CL; wherein the VH and CH1 of the first antigen-binding polypeptide and the VL and CL of the second antigen-binding polypeptide form a Fab specifically recognizing a first target antigen (e.g., CTLA-4, PD-L1, PD-L2, CD25, HER2, PD-1, CD3, CD4, CD123, or CD8); and (c) a third antigen-binding polypeptide comprising from N-terminus to C-terminus: an scFv (VH-VL, or VL-VH configuration) specifically recognizing a second target antigen (e.g., CTLA-4, PD-L1, PD-L2, CD25, HER2, PD-1, CD3, CD4, CD123, or CD8), a hinge region or portion thereof, and a second subunit of the Fc domain or portion thereof. See, e.g., FIG. 3C. In some embodiments, there is provided an immunocytokine comprising: (a) a first antigen-binding polypeptide comprising from N-terminus to C-terminus: a VH, a CH1, a cytokine or variant thereof (e.g., IL-2, IFN-α (e.g., IFN-α2b), IFN-γ, IL-10, IL-12, or IL-23) at a hinge region or portion thereof (e.g., any of SEQ ID NOs: 40-47, 50-52, and 55-59), and a portion (e.g., CH2) of a first subunit of an Fc domain (e.g., IgG1, IgG4, or effectorless IgG1); (b) a second antigen-binding polypeptide comprising from N-terminus to C-terminus: a VL, and a CL; (c) a third antigen-binding polypeptide comprising from N-terminus to C-terminus: a VH, a CH1, a hinge region or portion thereof, and a portion (e.g., CH2) of a second subunit of the Fc domain; and (d) a fourth antigen-binding polypeptide comprising from N-terminus to C-terminus: a VL, and a CL; wherein the VH and CH1 of the first antigen-binding polypeptide and the VL and CL of the second antigen-binding polypeptide form a first Fab specifically recognizing a first target antigen (e.g., CTLA-4, PD-L1, PD-L2, CD25, HER2, PD-1, CD3, CD4, CD123, or CD8), and wherein the VH and CH1 of the third antigen-binding polypeptide and the VL and CL of the fourth antigen-binding polypeptide form a second Fab specifically recognizing a second target antigen (e.g., CTLA-4, PD-L1, PD-L2, CD25, HER2, PD-1, CD3, CD4, CD123, or CD8). Other immunocytokine formats wherein the cytokine or variant thereof is positioned at a hinge region (e.g., within the hinge region, between the C-terminus of the antigen-binding fragment and the N-terminus of the hinge region, or between the C-terminus of the hinge region and the N-terminus of the Fc domain subunit) between an antigen-binding fragment (e.g., a ligand such as PD-L2 extracellular domain, a receptor, an antibody fragment such as VHH) and an Fc domain subunit or portion thereof are also contemplated herein. Any of the cytokine moiety and hinge configuration described for “full-length antibody immunocytokines” can be applied to any of the “partial antibody immunocytokines” or “partial Fc-fusion immunocytokines” described herein. In some embodiments, in the presence of binding of the antigen-binding protein or antigen binding fragment (e.g., ligand, receptor, VHH, scFv, or Fab) of the immunocytokine to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein or antigen binding fragment to the target antigen. In some embodiments, in the absence of binding of the antigen-binding protein or antigen-binding fragment (e.g., ligand, receptor, VHH, scFv, or Fab) of the immunocytokine to the target antigen, the activity (binding affinity to corresponding cytokine receptor or subunit thereof, and/or biological activity) of the cytokine or variant thereof positioned at the hinge region between the antigen-binding fragment (e.g., ligand, receptor, VHH, scFv, or Fab) and an Fc domain subunit (or portion thereof)) is no more than about 70% (such as no more than about any of 60%, 50%, 40%, 30%, 20%, 10%, 9%, 8%, 7%, 6%, 5%, 4%, 3%, 2%, %, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant. In some embodiments, the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, only one antigen-binding polypeptide comprising the Fc domain subunit or portion thereof comprises the cytokine or variant thereof positioned at the hinge region.

In some embodiments, each antigen-binding polypeptide comprising the Fc domain subunit or portion thereof comprises a cytokine or variant thereof positioned at the hinge region. In some embodiments, the immunocytokine comprises two or more (e.g., 2, 3, 4, 5, or more) cytokines or variants thereof, wherein the two or more cytokines or variant thereof are positioned in tandem (e.g., connected via an optional peptide linker) at the hinge region of one antigen-binding polypeptide. In some embodiments, the cytokine or variant thereof is a monomeric cytokine or variant thereof, such as IL-2 or IFN-α (e.g., IFN-α2a, IFN-α2b, IFN-α2c). In some embodiments, the cytokine or variant thereof is a dimeric cytokine or variant thereof. In some embodiments, the cytokine or variant thereof is a homodimeric cytokine or variant thereof, such as IL-10 or IFN-γ.

In some embodiments, the cytokine or variant thereof is a heterodimeric cytokine or variant thereof, such as IL-12 or IL-23. In some embodiments, both subunits of the dimeric cytokine or variant thereof are positioned in tandem (e.g., connected via an optional peptide linker) at the hinge region of one antigen-binding polypeptide. In some embodiments, one subunit of the dimeric cytokine or variant thereof is positioned at the hinge region of one antigen-binding polypeptide, and the other subunit of the dimeric cytokine or variant thereof is positioned at the hinge region of the other antigen-binding polypeptide. In some embodiments, the two or more cytokines or variants thereof are the same. In some embodiments, the two or more cytokines or variants thereof are different. In some embodiments, the immunocytokine is monospecific with regard to target antigen binding. In some embodiments, the immunocytokine is multispecific (e.g., bispecific) with regard to target antigen binding. In some embodiments, the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, erythropoietin, thrombopoietin, VEGF, G-CSF, M-CSF, SCF, and GM-CSF. In some embodiments, the cytokine or variant thereof is positioned between the C-terminus of CH1, scFv, or ligand/receptor and the N-terminus of the hinge region. In some embodiments, the cytokine or variant thereof is positioned between the C-terminus of the hinge region and the N-terminus of the Fc domain subunit or portion thereof. In some embodiments, the cytokine or variant thereof is positioned within the hinge region (e.g., replaces an internal portion of the hinge region, or inserted within the hinge region without deleting hinge region amino acid, or inserted within the hinge region and introducing one or more amino acids). In some embodiments, the Fc domain or portion thereof comprises knobs-into-holes (KIH) mutations. In some embodiments, the parental antigen-binding protein (e.g., antibody, antigen-binding fragment, or ligand/receptor-hinge-Fc fusion protein) is homodimeric. In some embodiments, the parental antigen-binding protein is heterodimeric. In some embodiments, the parental antigen-binding protein (e.g., antibody, or ligand-hinge-Fc fusion protein such as PD-L2-hinge-Fc fusion protein) is an agonist. In some embodiments, the parental antigen-binding protein is an antagonist.

### Cytokines or Variants Thereof

Cytokines (also referred to as “cytokine molecule” or “cytokine protein” interchangeably) are secreted proteins that modulate the activity of cells of the immune system. Examples of cytokines include the interleukins, interferons, chemokines, lymphokines, tumor necrosis factors, colony-stimulating factors for immune cell precursors, and so on. In some embodiments, the cytokine is a wildtype cytokine. In some embodiments, the cytokine is a naturally existing cytokine species variant. In some embodiments, the cytokine is a naturally existing cytokine subtype. A “cytokine variant” herein refers to any cytokine molecule that is not naturally existing, such as a cytokine active fragment (e.g., a cytokine fragment that retains at least about 10% biological activity or cytokine receptor binding activity of a full-length cytokine), a mutant, or a derivative thereof. A “cytokine or variant thereof” is also interchangeably referred to herein as a “cytokine moiety,” which can be a cytokine molecule, or a species variant, subtype, active fragment, mutant, or derivative thereof.

As used herein, “heterodimeric cytokine” or “cytokine heterodimer” refers to a cytokine consisting of two distinct protein subunits. At present, IL-12 family (includes IL-12, IL-23, IL-27, and IL-35) is the only naturally occurring heterodimeric cytokine family that is known. However, artificial heterodimeric cytokines can be constructed. For example, IL-6 and a soluble fragment of IL-6R can be combined to form a heterodimeric cytokine, as can CNTF and CNTF-R alpha (Trinchieri (1994) Blood 84:4008). “Homodimeric cytokine” or “cytokine homodimer” herein refers to a cytokine consisting of two identical protein subunits, such as IFN-γ or IL-10. “Monomeric cytokine” or “cytokine monomer” refers to a cytokine that consists of one unit of cytokine molecule. In some embodiments, the cytokine or variant thereof is a monomeric cytokine or variant thereof. In some embodiments, the cytokine or variant thereof is a homodimeric cytokine or variant thereof. In some embodiments, the cytokine or variant thereof is a heterodimeric cytokine or variant thereof.

In some embodiments, the cytokine moiety is a full-length cytokine molecule. In some embodiments, the cytokine moiety is a functional fragment of the cytokine molecule that is capable of producing some (e.g., at least about 10%, 20%, 30%, 40%, 50%, 60%, 70%, 80%, 90%, or 95%) or full biological activity and/or cytokine receptor binding activity of a full-length cytokine molecule. In some embodiments, the cytokine moiety is a precursor cytokine molecule. In some embodiments, the cytokine moiety is a mature cytokine molecule (e.g., no signal peptide). In some embodiments, the cytokine moiety is a wild-type cytokine. In some embodiments, the cytokine moiety is a naturally existing cytokine species variant. In some embodiments, the cytokine moiety is a naturally existing cytokine subtype. In some embodiments, the cytokine moiety is a cytokine variant, such as a mutant cytokine capable of producing some (e.g., at least about 10%, 20%, 30%, 40%, 50%, 60%, 70%, 80%, 90%, or 95%) or full biological activity and/or cytokine receptor binding activity of a wild-type cytokine. In some embodiments, the cytokine variant is a modified cytokine, such as glycosylated cytokine. The cytokine or variant thereof described herein can be a cytokine isolated from a variety of sources, such as from human tissue types or from another source, or prepared by recombinant or synthetic methods. In some embodiments, the cytokine moiety is a recombinant cytokine. In some embodiments, the cytokine moiety described herein can be a cytokine derived from any organism, such as mammals, including, but are not limited to, livestock animals (e.g., cows, sheep, goats, cats, dogs, donkeys, and horses), primates (e.g., human and non-human primates such as monkeys or chimpanzees), rabbits, and rodents (e.g., mice, rats, gerbils, and hamsters). In some embodiments, the cytokine moiety is a human cytokine, such as recombinant human cytokine. In some embodiments, the cytokine moiety is a murine cytokine, such as recombinant murine cytokine. In some embodiments, the cytokine moiety is a mature human cytokine. In some embodiments, the cytokine moiety comprises a signal peptide at the N-terminus of the cytokine molecule, the signal peptide is either from a different molecule or from the same cytokine molecule.

Cytokine variants can be of truncated versions, post-translationally modified versions, hybrid variants, peptide mimetics, biologically active fragments, deletion variants, substitution variants, or addition variants that maintain at least some degree (e.g., at least about 10%) of the parental cytokine activity (cytokine receptor binding activity and/or biological activity). “Parental cytokine” or “parent cytokine” described herein refers to the cytokine reference sequence from which the cytokine variant is engineered, modified, or derived from.

When immunocytokine of the subject invention is described to contain two or more different cytokines (and optionally including additional protein moieties), it means that the immunocytokine contains two or more different cytokine molecules (rather than two or more different cytokine subunits). For example, a homodimeric cytokine (e.g. IFN-α, IFN-β, IFN-γ, IL-5, IL-8, or the like) is referred to herein a single cytokine molecule. For example, an immunocytokine comprising two IL-5 monomers/subunits (either on the same polypeptide chain as a single-chain fusion or on different polypeptide chains), is considered to contain only one cytokine molecule, i.e., IL-5. Similarly, a heterodimeric cytokine such as IL-12, although it contains different subunits, is a single cytokine. For example, an immunocytokine comprising a p35 subunit and a p40 subunit (either on the same polypeptide chain as a single-chain fusion or on different polypeptide chains), is considered to contain only one cytokine molecule, i.e., IL-12. Furthermore, a heterodimeric form of normally homodimeric cytokines, such as a MCP-1/MCP-2 heterodimer, or of two alleles of a normally homodimeric cytokine (e.g., Zhang, J. Biol. Chem. [1994] 269:15918-24) is a single cytokine. In some embodiments, the cytokine subunit (e.g., p35 of IL-12) on one polypeptide chain of an immunocytokine can dimerize with the pairing cytokine subunit (e.g., p40) either on the same polypeptide chain or on a different polypeptide chain within the same immunocytokine. In some embodiments, the cytokine subunit (e.g., p35 of IL-12) of an immunocytokine can dimerize with the pairing cytokine subunit (e.g., p40) of a nearby immunocytokine.

In some embodiments, the cytokine variant comprises a mutation or modification (e.g., post-translational modification) that results in selectivity against a first type of receptor (e.g., trimeric receptor, or higher affinity receptor) versus a second type of receptor of the corresponding cytokine molecule (e.g., dimeric receptor, or weaker affinity receptor), measured as a ratio of activation of cells expressing the first type of receptor relative to activation of cells expressing the second type of receptor. For example, in some embodiments, the cytokine variant is a mutant IL-2 (or post-translationally modified IL-2), which binds IL-2Rβγ with stronger affinity (e.g., at least about any of 2, 3, 4, 5, 6, 7, 8, 9, or 10-fold stronger affinity) compared to IL-2Rαβγ, or activates cells expressing IL-2Rβγ more than (e.g., at least about any of 2, 3, 4, 5, 6, 7, 8, 9, or 10-fold activation) those expressing IL-2Rαβγ; or vice versa. In some embodiments, depending on the disease types to be treated, the preferred mutations or alterations increase cytokine moiety's activation of immune effector cells (e.g., CD8+ T cells for treating cancer). For example, in some embodiments, the IL-2 variant has a mutation (or post-translationally modification) that reduces the IL-2 variant's activation of cells expressing IL-2Rβγ receptor relative to the IL-2 variant's activation of cells expressing IL-2Rαβγreceptor.

In some embodiments, the mutation or modification of the cytokine variant leads to a differential effect (e.g., such as reduced binding or cell activation), compared to an immunocytokine without mutation or modification to such cytokine moiety. In one aspect, the differential effect is measured by the proliferative response of cells or cell lines that depend on the cytokine (e.g., IL-2) for growth. This response to the immunocytokine is expressed as an EC50 value, which is obtained from plotting a dose response curve and determining the protein concentration that results in a half-maximal response. In some embodiments, the ratio of the EC50 values obtained for cells expressing the first receptor type (e.g., IL-2Rβγ receptor) to cells expressing the second receptor type (e.g., IL-2Rαβγreceptor) for an immunocytokine of the invention (e.g., IL-2 variant immunocytokine) relative to the ratio of EC50 values for a reference immunocytokine (e.g., IL-2 wildtype immunocytokine of the same configuration) gives a measure of the differential effect for the immunocytokine. In some embodiments, the EC50 value obtained for an immunocytokine of the invention (e.g., IL-2 variant immunocytokine) relative to the EC50 value for a reference immunocytokine (e.g., IL-2 wildtype immunocytokine of the same configuration) gives a measure of the differential effect for the immunocytokine.

In some embodiments, the cytokine variant includes a mutation in one or more amino acids of the parental cytokine molecule (e.g., mature wildtype cytokine). In one embodiment, the cytokine variant includes an amino acid substitution at one or more amino acid positions in the cytokine. In another embodiment, the cytokine variant includes deletions or insertions of amino acids at one or more amino acid positions in the cytokine. In some embodiments, the cytokine variant includes modifications of one or more amino acids in the cytokine.

In some embodiments, the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, VEGF, erythropoietin, thrombopoietin, G-CSF, M-CSF, SCF, and GM-CSF, or natural variants or subtypes thereof. In some embodiments, the cytokine or variant thereof is an anti-inflammatory or immunosuppressive cytokine or variant thereof, such as IL-10, IL-27, IL-35, TGF-β, and VEGF. In some embodiments, the cytokine or variant thereof is a pro-inflammatory or immunostimulating cytokine or variant thereof, such as IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF. In some embodiments, the cytokine or variant thereof is selected from the group consisting of IL-2, IL-10, IL-12, IL-23, IFN-α (e.g., IFN-α2 or IFN-α2b), and IFN-γ. In some embodiments, the cytokine is IL-12, and the cytokine subunits are p35 and p40. In some embodiments, the cytokine is IL-23, and the cytokine subunits are p40 and p19. In some embodiments, the cytokine is IL-27, and the cytokine subunits are Epstein-Barr virus-induced gene 3 (EBI3) and IL-27p28. In some embodiments, the cytokine is IL-35, and the cytokine subunits are IL-12a (p35) and IL-270. In some embodiments, the cytokine variant is a single chain fusion of two or more subunits from different cytokines.

IL-2

In some embodiments, the cytokine or variant thereof is IL-2 or variant thereof. Interleukin-2 (IL-2), also known as T cell growth factor (TCGF), is a 15.5 kDa monomeric protein that plays a key role in lymphocyte generation, survival, and homeostasis. It is involved in body's natural response to microbial infection and discriminating between “self” and “non-self.” IL-2 is an interleukin, it belongs to a cytokine family that includes IL-4, IL-7, IL-9, IL-15, and IL-21. IL-2 mediates its effects by binding to IL-2 receptors (IL-2R) expressed on lymphocytes. Activated CD4⁺ T cells and activated CD8⁺ T cells are the major sources of IL-2. Its ability to expand lymphocyte populations and increase effector functions of these cells makes IL-2 an attractive therapy against cancer. IL-2 has been suggested for treating acute myeloid leukemia (AML), non-Hodgkin's lymphoma (NHL), cutaneous T-cell lymphoma (CTCL), breast cancer, and bladder cancer.

IL-2 receptor (IL-2R) is a complex consisting of three chains, a (CD25, p55), 13(CD122, p75), and γ (CD132, p65). The γ chain is shared by all IL-2 cytokine family members. IL-2 binding to either intermediate-affinity dimeric CD122/CD132 IL-2R (IL-2Rβγ, Kd˜10⁻⁹ M) or high-affinity trimeric CD25/CD122/CD132 IL-2R (IL-2Rαβγ, K_{d}˜10⁻⁴ M) can lead to signal transduction, while binding to CD25 alone cannot. The 1 chain is complexed with Janus kinase 1 (JAK1). The γ chain is complexed with JAK3. Upon IL-2 binding to IL-2R, JAK1 and JAK3 are activated and capable of adding phosphate groups to molecules, thus initiating three intracellular signaling pathways: the MAP kinase pathway, the Phosphoinositide 3-kinase (PI3K) pathway, and the JAK-STAT pathway. Dimeric IL-2Rβγ is expressed by memory CD8⁺ T cells, NK cells, and B cells, whereas high levels of trimeric IL-2Rαβγ is expressed by regulatory T cells (Tregs) and activated T cells.

Aldesleukin (Proleukin®), recombinant human IL-2, was the first cancer immunotherapy, and one of the first recombinant proteins, approved by the FDA in 1992. Currently, Aldesleukin is used for the treatment of metastatic renal cell carcinoma (mRCC) and metastatic melanoma (mM) by IV infusion. Due to the requirement of frequent intravenous infusion over multiple doses, administration of Aldesleukin occurs within a clinical setting. Aldesleukin has demonstrated complete cancer regression in about 10% of patients treated for metastatic melanoma and renal cancer (Klapper et al., Cancer, 2008; Rosenberg, Sci TranslMed., 2012; Smith et al., Clin Cancer Res., 2008). Approximately 70% of patients with complete responses have been cured, maintaining complete regression for more than 25 years after initial treatment (Atkins et al., J Clin Oncol., 1999; Klapper et al., Cancer, 2008; Rosenberg, Sci Transl Med., 2012; Rosenberg et al., Ann Surg., 1998; Smith et al., Clin Cancer Res., 2008). However, high doses of IL-2 can induce vascular leak syndrome (VLS), tumor tolerance caused by activation-induced cell death (AICD), and immunosuppression caused by the activation of Tregs. An additional concern of systemic IL-2 treatment is related to severe side effects upon intravenous administration, which include severe cardiovascular, pulmonary edema, hepatic, gastrointestinal (GI), neurological, and hematological events (Proleukin (aldesleukin) Summary of Product Characteristics [SmPC]: http://www.medicines.org.uk/emc/medicine/19322/SPC). The severe side effects often restrict optimal IL-2 dosing, which limits the number of patients who successfully respond to therapy. For more prevalent application in the future, toxicity and short half-life concerns of IL-2 need to be addressed.

Native human IL-2 precursor polypeptide consists of 153 amino acid residues (amino acids 1-20 are signal peptide), while the mature polypeptide consists of 133 amino acid residues (SEQ ID NO: 1). In some embodiments, the IL-2 moiety is a human mature IL-2. In some embodiments, the IL-2 moiety is a polypeptide substantially homologous to amino acid sequence of SEQ ID NO: 1, e.g., having at least about 85% (such as at least about any of 90%, 91%, 92%, 93%, 94%, 95%, 96%, 97%, 98%, 99%, or 100%) amino acid sequence identity to SEQ ID NO: 1. In some embodiments, the IL-2 moiety is not glycosylated. In some embodiments, the IL-2 moiety is glycosylated.

In some embodiments, the IL-2 moiety is (or consists essentially of) Aldesleukin (e.g., Proleukin®; see, e.g., https://www.drugbank.ca/drugs/DB00041). Aldesleukin (desalanyl-1, serine-125 human interleukin-2) is an antineoplastic (anticancer) biologic response modifier approved by the FDA. It has a molecular weight of approximately 15.3 kDa, and synonym recombinant interleukin-2 human, Interleukin-2 aldesleukin, 125-L-serine-2-133-interleukin 2 (human reduced), or Interleukin-2(2-133), 125-ser. Aldesleukin is a recombinant IL-2, it differs from native IL-2 in the following ways: a) Aldesleukin is not glycosylated because it is produced from E. coli; b) Aldesleukin has no N-terminal alanine (A); c) Aldesleukin has a cysteine to serine substitution at position 125 (C125S); and d) the aggregation state of Aldesleukin is likely different from that of native IL-2. Thus in some embodiments, the IL-2 variant comprises a cysteine to serine substitution at position 125 (C125S) from the human IL-2 mature form.

K. Sauvé et al. (Proc Natl Acad Sci USA. 1991; 88(11):4636-4640) found that amino acid residues K35, R38, F42, and K43 of wildtype IL-2 were found to be crucial for IL-2 receptor binding (IL-2Rα, low affinity form), and R38A and F24A mutations retained substantial IL-2 biological activity. R. Vazquez-Lombardi et al. (Nat Commun. 2017; 8:15373) discovered that R38D, K43E, and E61R mutations in IL-2 drove strong expansion of CD25⁻ cytotoxic subsets with minimal expansion of Tregs compared to wildtype IL-2. P65L mutation in IL-2 was found to have reduced systemic toxicity and greater antitumor efficacy compared to wildtype IL-2 (Chen et al., Cell Death Dis. 2018; 9(10):989).

In some embodiments, the IL-2 variant comprises one or more mutations at a position selected from the group consisting of F24, K35, R38, F42, K43, E61, and P65 relative to a wildtype IL-2 comprising the sequence of SEQ ID NO: 1. In some embodiments, the IL-2 variant comprises one or more mutations selected from the group consisting of F24A, R38D, K43E, E61R, and P65L relative to a wildtype IL-2 comprising the sequence of SEQ ID NO: 1. In some embodiments, the IL-2 variant comprises an R38D/K43E/E61R mutation relative to a wildtype IL-2 comprising the sequence of SEQ ID NO: 1. In some embodiments, the IL-2 variant comprises the sequence of SEQ ID NO: 2.

IFN-α

In some embodiments, the cytokine or variant thereof is IFN-α or variant thereof, such as IFN-α2 or variant thereof, or IFN-α2b or variant thereof. Human type I interferons (IFNs) are a large group of IFNs that help regulate the activity of the immune system. They bind to a specific cell surface receptor complex known as the IFN-α receptor (IFNAR) consisting of IFNAR1 and IFNAR2 chains. Mammalian type I IFNs contain IFN-α, IFN-β, IFN-κ, IFN-δ, IFN-ε, IFN-τ, IFN-ω, and IFN-ξ (a.k.a. limitin).

IFN-α proteins are mainly produced by plasmacytoid dendritic cells (pDCs), and mainly involved in innate immunity against viral infection. IFN-α proteins are 19-26 kDa monomeric proteins that have been extensively used for the treatment of cancer and viral diseases, such as Hepatitis B and C. There are 13 genes responsible for synthesis of 13 IFN-α subtypes: IFNA1, IFNA2, IFNA4, IFNA5, IFNA6, IFNA7, IFNA8, IFNA10, IFNA13, IFNA14, IFNA16, IFNA17, IFNA21.

Human IFN-α2a, IFN-α2b, and IFN-α2c represent allelic variants of the same gene. IFN-α2a and IFN-α2b have a lysine and an arginine at position 23 of the mature protein, respectively. Human IFN-α2a and IFN-α2b are the only IFN-α subtypes with an O-glycosylation site (on Thr106). Interferon alfa-2a (IFN-α2a; marketed by Hoffmann-La Roche as Roferon-A®) and interferon alfa-2b (IFN-α2b, recombinant form of IFN-α2; marketed by Schering-Plough as Intron-A®) have been approved for the treatment of hairy cell leukemia, melanoma, follicular lymphoma, renal cell carcinoma, AIDS-related Kaposi's sarcoma, and chronic myelogenous leukemia (M. Ferrantini et al., Biochimie. June-July 2007; 89(6-7):884-893). Recent studies have underscored new immunomodulatory effects of IFN-α, including activities on T cells and dendritic cells, which may lead to generation of a durable antitumor response. However, the use of IFN-α in clinical oncology is still generally based on exploiting the anti-proliferative and anti-angiogenic activities of these cytokines. Full exploit of the role of IFN-α as a regulator of immune response and tumor immunity would require novel approaches in the use of these cytokines. [0206] hIFN-α2b is a glycoprotein consisting of 166 amino acids with O-glycosylated threonine at position 106. Each rhIFN-2b consists of five a helices (called helix A to E) connected by a loop AB, BC, CD, and DE. Residues that are important in receptor binding are the AB loop (Arg22, Leu26, Phe27, Leu30, Lys31, Arg33, and His34), helix B (Ser68), helix C (Thr79, Lys83, Tyr85, and Tyr89), D helix (Arg120, lys121, Gln124, Lys131, and Glu132), and helix E (Arg144 and Glu146). Amino acid residues that are important in the biological activity are Leu30, Lys31, Arg33, His34, Phe36, Arg120, Lys121, Gln124, Tyrl22, Tyrl29, Lys131, Glu132, Arg144, and Glu146 (Ratih Asmana Ningrum, Scientifica (Cairo). 2014; 2014:970315).

In some embodiments, the IFN-α moiety is IFN-α2. In some embodiments, the IFN-α moiety is IFN-α2a. In some embodiments, the IFN-α moiety is IFN-α2b. In some embodiments, the IFN-α moiety is IFN-α2c. In some embodiments, the IFN-α moiety is a mature IFN-α. Native human IFN-α2b precursor polypeptide consists of 188 amino acid residues (amino acids 1-23 are signal peptide), while the mature polypeptide consists of 165 amino acid residues (SEQ ID NO: 3). In some embodiments, the IFN-α moiety is a polypeptide substantially homologous to amino acid sequence of SEQ ID NO: 3, e.g., having at least about 85% (such as at least about any of 90%, 91%, 92%, 93%, 94%, 95%, 96%, 97%, 98%, 99%, or 100%) amino acid sequence identity to SEQ ID NO: 3. In some embodiments, the IFN-α moiety is not glycosylated. In some embodiments, the IFN-α moiety is glycosylated.

In some embodiments, the IFN-α variant (e.g., IFN-α2b variant) comprises one or more mutations at a position selected from the group consisting of R22, L26, F27, L30, K31, D32, R33, H34, D35, F36, S68, T79, K83, Y85, Y89, R120, K121, Y122, Q124, Y129, K131, E132, R144, and E146 relative to an IFN-α (e.g., IFN-α2b) comprising the sequence of SEQ ID NO: 3. In some embodiments, the IFN-α variant (e.g., IFN-α2b variant) comprises one or more mutations selected from the group consisting of L30A, K31A, D32A, R33A, H34A, and D35A relative to an IFN-α comprising the sequence of SEQ ID NO: 3. In some embodiments, the IFN-α variant (e.g., IFN-α2b variant) comprises the sequence of any of SEQ ID NOs: 4-9. In some embodiments, the IFN-α variant (e.g., IFN-α2b variant) comprises an L30A mutation relative to an IFN-α comprising the sequence of SEQ ID NO: 3. In some embodiments, the IFN-α variant (e.g., IFN-α2b variant) comprises the sequence of SEQ ID NO: 4.

IFN-γ

In some embodiments, the cytokine or variant thereof is IFN-γ or variant thereof. Interferon gamma (IFNγ) is a disulfide-linked dimerized soluble cytokine that is the only member of the type II class of interferons. IFN-γ is a homodimer of −25 kDa with a tertiary fold built around an unusual pattern of interdigitating a helices. It is produced predominantly by T cells and NK cells in response to a variety of inflammatory or immune stimuli. IFN-γ can serve both as an immune system activator and suppressor. Studies showed that cancer immunotherapy (checkpoint inhibitors) acts partially through an increase of IFN-γ expression, leading to the elimination of cancer cells. Resistance to immunotherapy is attributed to defects in IFN-γ signaling. However, IFN-γ can also contribute to cancer evasion by promoting tumorigenesis and angiogenesis, eliciting expression of tolerant molecules such as PD-L1, and inducing homeostasis program. Due to its opposite and competing effects on the immune system, IFN-γ has not been approved by FDA to treat cancer patients except in the case of malignant osteoporosis (L. Ni and J. Lu, CancerMed. 2018; 7(9):4509-4516).

Monomeric native human IFN-γ (hIFN-γ) pre-pro-polypeptide consists of 166 amino acid residues (amino acids 1-23 are signal peptide); the monomeric mature polypeptide consists of 138 amino acid residues (SEQ ID NO: 10), corresponding to amino acids 24-161 of the pre-pro-polypeptide; amino acids 162-166 are propeptide sequence of the pre-pro-polypeptide. In some embodiments, the monomeric IFN-γ moiety is a monomeric mature IFN-γ. In some embodiments, the monomeric IFN-γ moiety is a polypeptide substantially homologous to amino acid sequence of SEQ ID NO: 10, e.g., having at least about 85% (such as at least about any of 90%, 91%, 92%, 93%, 94%, 95%, 96%, 97%, 98%, 99%, or 100%) amino acid sequence identity to SEQ ID NO: 10. In some embodiments, the IFN-γ moiety (or subunit) is not glycosylated. In some embodiments, the IFN-γ moiety (or subunit) is glycosylated. In some embodiments, the IFN-γ moiety comprises two identical IFN-γ monomers/subunits. In some embodiments, the IFN-γ moiety comprises two different IFN-γ monomers/subunits. For example, in some embodiments, the IFN-γ moiety comprises one wildtype IFN-γ monomer and one IFN-γ variant monomer. In some embodiments, the IFN-γ moiety comprises two IFN-γ monomers (e.g., two IFN-γ variant or wildtype monomers) linked together, such as via a peptide linker (e.g., any of SEQ ID NOs: 227-229) or a chemical linker.

IFN-γ amino acid residues S20, A23, H111, and Q115 are important for receptor binding; amino acid residues V5, S20, A23, G26, and H111 are important for IFN-γ biological activity (M. Randal and A. A. Kossiakoff, Structure. 2001; 9(2):155-63). Lander et al. (J Mol Biol. 2000; 299(1):169-79) developed a biologically active single chain variant of IFN-γ (IFN-γSC1), by linking two monomeric IFN-γ with a 7-amino acid residue linker and changing His 111 in the first IFN-γ monomer to an aspartic acid residue. Due to the H111D mutation, IFN-γSC1 can only bind one IFN-γRα but can fully retain its biological activity in cell proliferation, MHC class I induction, and anti-viral assays.

In some embodiments, the monomeric IFN-γ comprises the sequence of SEQ ID NO: 10. In some embodiments, the IFN-γ variant comprises one or more mutations within one or both IFN-γ subunits at a position selected from the group consisting of V5, S20, D21, V22, A23, D24, N25, G26, H111, and Q115 relative to a wildtype IFN-γ subunit comprising the sequence of SEQ ID NO: 10. In some embodiments, the IFN-γ variant comprises one or more mutations within one or both IFN-γ subunits selected from the group consisting of S20A, D21A, D21K, V22A, A23S, A23E, A23Q, A23V, D24A, D24E, N25A, N25K, and HI ID relative to a wildtype IFN-γ subunit comprising the sequence of SEQ ID NO: 10. In some embodiments, the IFN-γ variant comprises one or more mutations within one or both IFN-γ subunits selected from the group consisting of S20A/D21A, D21K, V22A/A23S, D24A/N25A, A23E/D24E/N25K, A23Q, and A23V relative to a wildtype IFN-γ subunit comprising the sequence of SEQ ID NO: 10. In some embodiments, one or both subunits of the IFN-γ variant comprises the sequence of any of SEQ ID NOs: 11-17. In some embodiments, the IFN-γ variant comprises an A23V mutation within one or both IFN-γ subunits relative to a wildtype IFN-γ subunit comprising the sequence of SEQ ID NO: 10. In some embodiments, the one or both subunits of the IFN-γ variant comprises the sequence of SEQ ID NO: 13. In some embodiments, the two subunits of the IFN-γ or variant thereof are connected by a linker (e.g., any of SEQ ID NOs: 227-229). In some embodiments, the IFN-γ variant comprises the sequence of SEQ ID NO: 19. In some embodiments, both subunits of the IFN-γ comprises the sequence of SEQ ID NO: 10. In some embodiments, the IFN-γ moiety is a recombinant “wildtype” IFN-γ comprising two wildtype IFN-γ subunits connected by a linker (e.g., any of SEQ ID NOs: 227-229), such as comprising the sequence of SEQ ID NO: 18.

IL-10

In some embodiments, the cytokine or variant thereof is IL-10 or variant thereof. Interleukin 10 (IL-10) is an α-helical cytokine that is expressed as a non-covalently linked homodimer of ˜37 kDa, also known as human cytokine synthesis inhibitory factor (CSIF). It plays a key role in the induction and maintenance of tolerance. IL-10 signals through a JAK-STAT complex. The IL-10 receptor (IL-10R) has two subunits, an a subunit that is primarily expressed on immune cells, particularly monocytes and macrophages with the highest expression, and an ubiquitously expressed p subunit. IL-10 is mainly produced by monocytes and, to a lesser extent, lymphocytes, including type-II T helper cells (TH2), mast cells, CD4⁺CD25⁺Foxp3⁺ regulatory T cells, and subsets of activated T cells and B cells. Dendritic cells and NK cells can also produce IL-10. IL-10 suppresses the secretion of pro-inflammatory cytokines like TNFα, IL-1, IL-6, IL-12 as well as Th1 cytokines such as IL-2 and IFN-γ and controls differentiation and proliferation of macrophages, B-cells and T-cells (Glocker, E. O. et al., Ann. N.Y. Acad. Sci. 1246, 102-107 (2011); Moore, K. W. et al., Annu. Rev. Immunol. 19, 683-765 (2001); R. de Waal Malefyt et al., J. Exp. Med. 174, 915-924 (1991); Williams, L. M. et al., Immunology 113, 281-292 (2004)). Moreover, it is a potent inhibitor of antigen presentation, inhibiting MHC II expression as well as upregulation of co-stimulatory molecules CD80 and CD86 (Mosser, D. M. & Zhang, X. Immunological Reviews 226, 205-218 (2008)). If IL-10 is not present or not functional, inflammation cannot be controlled. This makes IL-10 an attractive therapeutic candidate for autoimmune diseases. However, clinical trials using IL-10 and the development of a recombinant IL-10 (ilodecakin, TENOVIL®, Schering-Plough Research Institue, Kenilworth, N.J.) have been discontinued due to lack of efficacy. Recent studies have shed light on IL-10's potential role in tumor treatment (Fujii et al., (October 2001). “Interleukin-10 promotes the maintenance of antitumor CD8(+) T-cell effector function in situ”. Blood. 98(7):2143-51).

Monomeric native human IL-10 precursor polypeptide consists of 178 amino acid residues (amino acids 1-18 are signal peptide), while the monomeric mature IL-10 polypeptide consists of 160 amino acid residues (SEQ ID NO: 20). In some embodiments, the monomeric IL-10 moiety is a monomeric mature IL-10. In some embodiments, the monomeric IL-10 moiety is a polypeptide substantially homologous to amino acid sequence of SEQ ID NO: 20, e.g., having at least about 85% (such as at least about any of 90%, 91%, 92%, 93%, 94%, 95%, 96%, 97%, 98%, 99%, or 100%) amino acid sequence identity to SEQ ID NO: 20. In some embodiments, the IL-10 moiety (or subunit) is not glycosylated. In some embodiments, the IL-10 moiety (or subunit) is glycosylated. In some embodiments, the IL-10 moiety comprises two identical IL-10 monomers/subunits. In some embodiments, the IL-10 moiety comprises two different IL-10 monomers/subunits. For example, in some embodiments, the IL-10 moiety comprises one wildtype IL-10 monomer and one IL-10 variant monomer. In some embodiments, the IL-10 moiety comprises two IL-10 monomers (e.g., two IL-10 variant or wildtype monomers) linked together, such as via a peptide linker (e.g., any of SEQ ID NOs: 227-229) or a chemical linker, see e.g., a biologically active single chain IL-10 in US20130316404, the content of which is incorporated herein by reference in its entirety.

IL-10 amino acid residues N21, M22, R24, R32, H90, S31, and S93 are important in IL-10 receptor binding; residue R24 is crucial for IL-10 biological activity (Yoon et al., J Biol Chem. 2006; 281(46):35088-35096; E. S. Acuner-Ozbabacan et al. BMC Genomics. 2014; 15 Suppl 4(Suppl 4):S2).

In some embodiments, the monomeric IL-10 comprises the sequence of SEQ ID NO: 20. In some embodiments, the IL-10 variant comprises one or more mutations within one or both IL-10 subunits at a position selected from the group consisting of N21, M22, R24, D25, L26, R27, D28, A29, F30, S31, R32, H90, and S93 relative to a wildtype IL-10 subunit comprising the sequence of SEQ ID NO: 20. In some embodiments, the IL-10 variant comprises one or more mutations within one or both IL-10 subunits selected from the group consisting of R24A, D25A, L26A, R27A, D28A, A29S, F30A, S31A, and R32A relative to a wildtype IL-10 subunit comprising the sequence of SEQ ID NO: 20. In some embodiments, the IL-10 variant comprises one or more mutations within one or both IL-10 subunits selected from the group consisting of R24A, D25A/L26A, R27A, D28A/A29S, F30A/S31A, and R32A relative to a wildtype IL-10 subunit comprising the sequence of SEQ ID NO: 20. In some embodiments, the one or both subunits of the IL-10 variant comprises the sequence of any of SEQ ID NOs: 21-26. In some embodiments, the IL-10 variant comprises an R27A mutation within one or both IL-10 subunits relative to a wildtype IL-10 subunit comprising the sequence of SEQ ID NO: 20. In some embodiments, the one or both subunits of the IL-10 variant comprises the sequence of SEQ ID NO: 23. In some embodiments, the two subunits of the IL-10 or variant thereof are connected by a linker. In some embodiments, the IL-10 variant comprises the sequence of SEQ ID NO: 28. In some embodiments, both subunits of IL-10 comprises the sequence of SEQ ID NO: 20. In some embodiments, the IL-10 moiety is a recombinant “wildtype” IL-10 comprising two wildtype IL-10 monomers connected by a linker (e.g., any of SEQ ID NOs: 227-229), such as comprising the sequence of SEQ ID NO: 27.

IL-12

In some embodiments, the cytokine or variant thereof is IL-12 or variant thereof. IL-12 is a 70 kDa heterodimeric protein consisting of two covalently (disulfide bond) linked p35 (IL-12A) and p40 (IL-12B) subunits. P40 subunit is shared between IL-12 and IL-23. The active heterodimer (referred to as “p70”), and a homodimer of p40 are formed following protein synthesis. IL-12 is an interleukin belonging to the IL-12 family, which is the only family comprising heterodimeric cytokines, including IL-12, IL-23, IL-27, and IL-35. IL-12 is produced by dendritic cells, macrophages, neutrophils, and human B-lymphoblastoid cells (NC-37) in response to antigenic stimulation. IL-12 functions by binding to the IL-12 receptor (IL-12R), which is a heterodimeric receptor formed by IL-12Rβ1 and IL-12Rβ2, and in turn leading to JAK-STAT pathway activation. IL-12 promotes the development of Th1 responses and greatly induces IFNγ production by T and NK cells. IL-12's ability to activate both innate (NK cells) and adaptive (cytotoxic T lymphocytes) immunities has made it a promising candidate for cancer immunotherapy. Despite positive results from animal trials, IL-12 has only showed modest anti-tumor responses in clinical trials and was often accompanied by significant issues with toxicity (Lasek et al., Cancer Immunol Immunother., 2014). Treatment with IL-12 was associated with systemic flu-like symptoms (fever, chills, fatigue, erythromelalgia, or headache) and toxic effects on the bone marrow and liver. Dosing studies showed that patients could only tolerate doses under 1 μg/kg, far below the therapeutic dose. The result is that clinical trials with IL-12—used either as monotherapy or combined with other agents—failed to demonstrate potent sustained therapeutic efficacy ((Lasek et al., Cancer Immunol Immunother., 2014).

Native human p35 (IL-12A) precursor polypeptide consists of 219 amino acid residues (amino acids 1-22 are signal peptide), while the mature polypeptide consists of 197 amino acid residues (SEQ ID NO: 29). Native human p40 (IL-12B) precursor polypeptide consists of 328 amino acid residues (amino acids 1-22 are signal peptide), while the mature polypeptide consists of 306 amino acid residues (SEQ ID NO: 30). In some embodiments, the IL-12 moiety (or IL-12 subunit) is a mature IL-12 (or mature subunit). In some embodiments, the IL-12A (p35) subunit or variant thereof is a polypeptide substantially homologous to amino acid sequence of SEQ ID NO: 29, e.g., having at least about 85% (such as at least about any of 90%, 91%, 92%, 93%, 94%, 95%, 96%, 97%, 98%, 99%, or 100%) amino acid sequence identity to SEQ ID NO: 29. In some embodiments, the IL-12B (p40) subunit or variant thereof is a polypeptide substantially homologous to amino acid sequence of SEQ ID NO: 30, e.g., having at least about 85% (such as at least about any of 90%, 91%, 92%, 93%, 94%, 95%, 96%, 97%, 98%, 99%, or 100%) amino acid sequence identity to SEQ ID NO: 30. In some embodiments, the IL-12 (or subunit) or variant thereof is not glycosylated. In some embodiments, the IL-12 (or subunit) or variant thereof is glycosylated. In some embodiments, the IL-12 variant comprises one wildtype subunit (e.g., wt p35) and one mutant subunit (e.g., variant p40). In some embodiments, the IL-12 variant comprises two variant subunits (p35 variant and p40 variant). In some embodiments, the IL-12 variant comprises two wildtype subunits (e.g., wt p35 and p40) that are linked together via a synthetic peptide linker (e.g., any of SEQ ID NOs: 227-229) or a chemical linker.

Within the p40 subunit, amino acid residues that are important for IL-12 receptor binding are C177, E45, E59, and D62 (Luo et al. J MolBiol. 2010; 402(5):797-812). Studies suggested that an accessible N terminus of the p40 subunit is important for IL-12 bioactivity. Lieschke et al. constructed a single chain IL-12 (scIL-12) and noted that the order of the subunits affected IL-12 biologic activity: when the p35 subunit was at the N-terminus of p40 subunit, IL-12 activity greatly decreased; when p40 subunit was at the N-terminus of the p35 subunit, scIL-12 had biological activity comparable to rIL-12 (Lieschke et al. Nat Biotechnol. 1997; 15(1):35-40).

In some embodiments, the IL-12 moiety comprises a wildtype p35 subunit (SEQ ID NO: 29). In some embodiments, the IL-12 moiety comprises a variant p35 subunit. In some embodiments, the IL-12 moiety comprises a wildtype p40 subunit (SEQ ID NO: 30). In some embodiments, the IL-12 moiety comprises a variant p40 subunit. In some embodiments, the IL-12 moiety comprises a wildtype or variant p35 subunit and a wildtype or variant p40 subunit connected by a peptide linker (e.g., any of SEQ ID NOs: 227-229). In some embodiments, the IL-12 moiety comprises from N-terminus to C-terminus: wildtype or variant p40 subunit—linker (e.g., any of SEQ ID NOs: 227-229)—wildtype or variant p35 subunit. In some embodiments, the IL-12 moiety comprises from N-terminus to C-terminus: wildtype or variant p35 subunit—linker (e.g., any of SEQ ID NOs: 227-229)—wildtype or variant p40 subunit. In some embodiments, the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30. In some embodiments, the IL-12 variant comprises one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30. In some embodiments, the p40 subunit of the IL-12 variant comprises the sequence of any of SEQ ID NOs: 31-34. In some embodiments, the IL-12 variant comprises an E59A/F60A mutation within the p40 subunit relative to a wildtype p40 subunit comprising the sequence of SEQ ID NO: 30. In some embodiments, the p40 subunit of the IL-12 variant comprises the sequence of SEQ ID NO: 31. In some embodiments, the IL-12 variant comprises an F60A mutation within the p40 subunit relative to a wildtype p40 subunit comprising the sequence of SEQ ID NO: 30. In some embodiments, the p40 subunit of the IL-12 variant comprises the sequence of SEQ ID NO: 33. In some embodiments, the p40 subunit and the p35 subunit of the IL-12 or variant thereof are connected by a linker. In some embodiments, the IL-12 variant comprises the sequence of SEQ ID NO: 36 or 275. In some embodiments, the IL-12 moiety is a recombinant “wildtype” IL-12 comprising a wildtype p35 subunit and a wildtype p40 subunit connected by a linker (e.g., any of SEQ ID NOs: 227-229), such as comprising the sequence of SEQ ID NO: 35.

IL-23

In some embodiments, the cytokine or variant thereof is IL-23 or variant thereof. Interleukin-23 (IL-23) belongs to the IL-12 cytokine family, is a heterodimeric cytokine consisting of an IL12B (p40) subunit (shared with IL-12) and the IL23A (p19) subunit. IL-23 functions through binding to IL-23 receptor composed of IL-12R β1 and IL-23R (p19 subunit binds IL-23R while p40 subunit binds IL-12Rβ1), resulting in Janus kinase 2 and Tyrosine kinase 2 kinases recruitment and phosphorylation of STAT3 and STAT4, leading to gene activation. STAT3 is responsible for key Th17 development characteristics such as RORγt expression, or transcription of Th17 cytokines such as IL-17, IL-21, IL-22, and GM-CSF which mediate protection against fungi and bacteria and participate in barrier immunity. IL-23 is mainly secreted by activated dendritic cells, macrophages or monocytes stimulated by antigen stimulus. IL-23 receptor is expressed on Th17 and NK cells. It was found that autoimmune and cancerous diseases are associated with IL-23 imbalance and increase. The most important function of IL-23 is its role in the development and differentiation of effector Th17 cells. In the context of chronic inflammation, activated DCs and macrophages produce IL-23, which promotes the development of Th17 cells. Autoimmune diseases such as psoriasis, Crohn's disease, rheumatoid arthritis, or multiple sclerosis have recently been found to be associated with IL-23-mediated signaling promoted by IL-23 receptor-expressing TH-17 and other lymphocyte subsets.

Native human p19 (IL-23A) precursor polypeptide consists of 189 amino acid residues (amino acids 1-19 are signal peptide), while the mature polypeptide consists of 170 amino acid residues (SEQ ID NO: 37). Native human p40 (IL-12B) precursor polypeptide consists of 328 amino acid residues (amino acids 1-22 are signal peptide), while the mature polypeptide consists of 306 amino acid residues (SEQ ID NO: 30). In some embodiments, the IL-23 moiety (or IL-23 subunit) is a mature IL-23 (or IL-23 mature subunit). In some embodiments, the IL-23A (p19) or variant thereof is a polypeptide substantially homologous to amino acid sequence of SEQ ID NO: 37, e.g., having at least about 85% (such as at least about any of 90%, 91%, 92%, 93%, 94%, 95%, 96%, 97%, 98%, 99%, or 100%) amino acid sequence identity to SEQ ID NO: 37. In some embodiments, the IL-12B (p40) subunit or variant thereof is a polypeptide substantially homologous to amino acid sequence of SEQ ID NO: 30, e.g., having at least about 85% (such as at least about any of 90%, 91%, 92%, 93%, 94%, 95%, 96%, 97%, 98%, 99%, or 100%) amino acid sequence identity to SEQ ID NO: 30. In some embodiments, the IL-23 (or subunit) or variant thereof is not glycosylated. In some embodiments, the IL-23 (or subunit) or variant thereof is glycosylated. In some embodiments, the IL-23 variant comprises one wildtype subunit (e.g., wt p19) and one mutant subunit (e.g., variant p40). In some embodiments, the IL-23 variant comprises two variant subunits (p19 variant and p40 variant). In some embodiments, the IL-23 variant comprises two wildtype subunits (e.g., wt p19 and p40) that are linked together via a synthetic peptide linker (e.g., any of SEQ ID NOs: 227-229) or a chemical linker. Within the p40 subunit, amino acid residues that are important for IL-23 receptor binding are C177, E45, E59, and D62 (Luo et al. J Mol Biol. 2010; 402(5):797-812).

In some embodiments, the IL-23 moiety comprises a wildtype p19 subunit (SEQ ID NO: 37). In some embodiments, the IL-23 moiety comprises a variant p19 subunit. In some embodiments, the IL-23 moiety comprises a wildtype p40 subunit (SEQ ID NO: 30). In some embodiments, the IL-23 moiety comprises a variant p40 subunit. In some embodiments, the IL-23 moiety comprises a wildtype or variant p19 subunit and a wildtype or variant p40 subunit connected by a peptide linker (e.g., any of SEQ ID NOs: 227-229). In some embodiments, the IL-23 moiety comprises from N-terminus to C-terminus: wildtype or variant p40 subunit—linker (e.g., any of SEQ ID NOs: 227-229)—wildtype or variant p19 subunit. In some embodiments, the IL-23 moiety comprises from N-terminus to C-terminus: wildtype or variant p19 subunit—linker (e.g., any of SEQ ID NOs: 227-229)—wildtype or variant p40 subunit. In some embodiments, the IL-23 variant comprises one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30. In some embodiments, the IL-23 variant comprises one or more mutations within the p40 subunit selected from the group consisting of Q56A, V57A, K58A, E59A, F60A, G61A, D62A, A63S, G64A, and Q65A relative to a wildtype p40 subunit comprising the sequence of SEQ ID NO: 30. In some embodiments, the p40 subunit of the IL-23 variant comprises the sequence of any of SEQ ID NOs: 31-34. In some embodiments, the IL-23 variant comprises an E59A/F60A mutation within the p40 subunit relative to a wildtype p40 subunit comprising the sequence of SEQ ID NO: 30. In some embodiments, the p40 subunit of the IL-23 variant comprises the sequence of SEQ ID NO: 31. In some embodiments, the p40 subunit and the p19 subunit of the IL-23 or variant thereof are connected by a linker (e.g., any of SEQ ID NOs: 227-229). In some embodiments, the IL-23 variant comprises the sequence of SEQ ID NO: 39. In some embodiments, the IL-23 moiety is a recombinant “wildtype” IL-23 comprising a wildtype p35 subunit and a wildtype p40 subunit connected by a linker (e.g., any of SEQ ID NOs: 227-229), such as comprising the sequence of SEQ ID NO: 38.

IL-17

In some embodiments, the cytokine or variant thereof is IL-17 or variant thereof. The IL-17 family comprises IL17A, IL-17B, IL-17C, IL-17D, IL-17E (a.k.a. IL-25) and IL-17F. Interleukin 17A (IL-17A or IL-17) is a disulfide-linked, homodimeric, secreted glycoprotein with a molecular mass of about 35 kDa. Each subunit of the homodimer is approximately 15-20 KDa. IL-17A is a pro-inflammatory cytokine produced by T helper 17 (Th17) cells in response to their stimulation with IL-23. IL-17 interacts with IL-17R and activates several signaling cascades that, in turn, lead to the induction of chemokines. These chemokines act as chemoattractant to recruit immune cells, such as monocytes and neutrophils to the site of inflammation.

### Activities of Cytokines or Variants Thereof

The “activity” of a cytokine or a variant thereof described herein comprises the binding affinity of the cytokine or a variant thereof to corresponding cytokine receptor; and/or the biological activity (or bioactivity) of the cytokine or a variant thereof, such as inducing or inhibiting signal transduction, inducing or inhibiting cell proliferation, differentiation, and/or activation, inducing or inhibiting the secretion of effecting cytokine(s) (e.g., pro-inflammatory cytokines), etc., upon cytokine/cytokine receptor binding. These biological activities are also referred to herein as direct biological activities. In some embodiments, the biological activity of a cytokine or a variant thereof also comprises indirect biological activities, such as any biological activity resulting from the direct biological activities. For example, in some embodiments, the biological activity also comprises cancer cell killing by immune cells attracted to the tumor site due to the secreted effecting cytokines, such as inflammatory markers IL-6, IP-2 (GRO-0)/CXCL2, G-CSF/CSF3, TIMP-1, KC (GRO-α)/CXCL1, etc.

In some embodiments, in the presence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody, or ligand/receptor-hinge-Fc fusion protein) or antigen-binding fragment to the target antigen. In some embodiments, in the presence of binding of the antigen-binding protein (e.g., antibody (e.g., full-length antibody), or ligand/receptor-hinge-Fc fusion protein) or antigen-binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine or variant thereof increases to at least about 2-fold (such as at least about any of 3, 4, 5, 6, 7, 8, 9, 10, 20, 30, 40, 50, 60, 70, 80, 90, or 100-fold) of that in the absence of binding of the antigen-binding protein (e.g., antibody, or ligand/receptor-hinge-Fc fusion protein) or antigen-binding fragment to the target antigen.

In some embodiments, in the absence of binding of the antigen-binding protein (e.g., antibody (e.g., full-length antibody), or ligand/receptor-hinge-Fc fusion protein) or antigen-binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine or variant thereof positioned at the hinge region of the antigen-binding polypeptide (such as positioned at the hinge region of a heavy chain of an antibody (e.g., full-length antibody), or positioned at the hinge region between an antigen binding fragment (e.g., ligand, receptor, VHH, scFv, or Fab) and an Fc domain subunit (or portion thereof), see FIGS. 2A-4C) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state.

In some embodiments, the “corresponding cytokine or variant thereof” is the same as the cytokine or variant thereof positioned at the hinge region, but expressed under a different state or at a different position. A cytokine or variant thereof “in a free state” herein refers to a cytokine or variant thereof in a soluble form, without attaching to any moiety such as cell membrane or another molecule (e.g., Fc fragment, or N-terminus or C-terminus of a full-length antibody or antigen binding fragment (e.g., ligand, receptor, VHH, scFv, or Fab)).

In some embodiments, in the absence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine or variant thereof positioned at the hinge region of the antigen-binding polypeptide (such as positioned at the hinge region of a heavy chain of an antibody (e.g., full-length antibody), or positioned at the hinge region between an antigen binding fragment (e.g., ligand, receptor, VHH, scFv, or Fab) and an Fc domain subunit (or portion thereof), see FIGS. 2A-4C)) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof in a free state; and in the presence of binding of the antigen-binding protein (e.g., antibody (e.g., full-length antibody), or ligand/receptor-hinge-Fc fusion protein) or antigen-binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody (e.g., full-length antibody), or ligand/receptor-hinge Fc fusion protein) or antigen-binding fragment (e.g., ligand, receptor, VHH, scFv, or Fab) to the target antigen.

In some embodiments, in the absence of binding of the full-length antibody to the target antigen, the activity (binding affinity to corresponding cytokine receptor or subunit thereof, and/or biological activity) of the cytokine or variant thereof positioned at the hinge region of a heavy chain of the full-length antibody is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc domain subunit of the full-length antibody. In some embodiments, in the absence of binding of the antigen-binding fragment (e.g., scFv or Fab) to the target antigen, the activity (binding affinity to corresponding cytokine receptor or subunit thereof, and/or biological activity) of the cytokine or variant thereof positioned at the hinge region between the antigen binding fragment (e.g., scFv or Fab) and an Fc domain subunit (or portion thereof) (see FIGS. 3A-3C) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof expressed at any of: i) the N-terminus of a VH of the antigen binding fragment (e.g., scFv or Fab), ii) the N-terminus of a VL of the antigen binding fragment (e.g., scFv or Fab), iii) the C-terminus of the Fc domain subunit (or portion thereof), iv) the C-terminus of a CL of the antigen binding fragment (Fab), and v) the N-terminus of the Fc domain subunit. In some embodiments, in the absence of binding of the antigen-binding fragment (e.g., VHH, ligand, or receptor) to the target antigen, the activity (binding affinity to corresponding cytokine receptor or subunit thereof, and/or biological activity) of the cytokine or variant thereof positioned at the hinge region between the antigen binding fragment (e.g., VHH, ligand, or receptor) and an Fc domain subunit (or portion thereof) (see FIGS. 4A-4C) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.10%, or 0%) of that of a corresponding cytokine or variant thereof expressed at any of: i) the N-terminus of the antigen-binding fragment (e.g., VHH, ligand, or receptor), ii) the C-terminus of the Fc domain subunit (or portion thereof), and iii) the N-terminus of the Fc domain subunit.

In some embodiments, in the presence of binding of the full-length antibody to the target antigen, the activity (binding affinity to corresponding cytokine receptor or subunit thereof, and/or biological activity) of the cytokine or variant thereof positioned at the hinge region of a heavy chain of the full-length antibody is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc subunit of the full-length antibody. In some embodiments, in the presence of binding of the antigen binding fragment (e.g., scFv or Fab) to the target antigen, the activity (binding affinity to corresponding cytokine receptor or subunit thereof, and/or biological activity) of the cytokine or variant thereof positioned at the hinge region between the antigen binding fragment (e.g., scFv or Fab) and an Fc domain subunit (or portion thereof) (see FIGS. 3A-3C) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of a VH of the antigen binding fragment (e.g., scFv or Fab), ii) the N-terminus of a VL of the antigen binding fragment (e.g., scFv or Fab), iii) the C-terminus of the Fc domain subunit (or portion thereof), iv) the C-terminus of a CL of the antigen binding fragment (Fab), and v) the N-terminus of the Fc domain subunit. In some embodiments, in the presence of binding of the antigen binding fragment (e.g., VHH, ligand, or receptor) to the target antigen, the activity (binding affinity to corresponding cytokine receptor or subunit thereof, and/or biological activity) of the cytokine or variant thereof positioned at the hinge region between the antigen binding fragment (e.g., VHH, ligand, or receptor) and an Fc domain subunit (or portion thereof) (see FIGS. 4A-4C) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of the antigen-binding fragment (e.g., VHH, ligand, or receptor), ii) the C-terminus of the Fc domain subunit (or portion thereof), and iii) the N-terminus of the Fc domain subunit.

In some embodiments, in the absence of binding of the full-length antibody to the target antigen, the activity (binding affinity to corresponding cytokine receptor or subunit thereof, and/or biological activity) of the cytokine or variant thereof positioned at the hinge region of a heavy chain of the full-length antibody is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc domain subunit of the full-length antibody; and in the presence of binding of the full-length antibody to the target antigen, the activity (binding affinity to corresponding cytokine receptor or subunit thereof, and/or biological activity) of the cytokine or variant thereof positioned at the hinge region of a heavy chain of the full-length antibody is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc subunit of the full-length antibody. In some embodiments, in the absence of binding of the antigen binding fragment (e.g., scFv or Fab) to the target antigen, the activity (binding affinity to corresponding cytokine receptor or subunit thereof, and/or biological activity) of the cytokine or variant thereof positioned at the hinge region between the antigen binding fragment (e.g., scFv or Fab) and an Fc domain subunit (or portion thereof) (see FIGS. 3A-3C) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof expressed at any of: i) the N-terminus of a VH of the antigen binding fragment (e.g., scFv or Fab), ii) the N-terminus of a VL of the antigen binding fragment (e.g., scFv or Fab), iii) the C-terminus of the Fc domain subunit (or portion thereof), iv) the C-terminus of a CL of the antigen binding fragment (Fab), and v) the N-terminus of the Fc domain subunit; and in the presence of binding of the antigen binding fragment (e.g., scFv or Fab) to the target antigen, the activity (binding affinity to corresponding cytokine receptor or subunit thereof, and/or biological activity) of the cytokine or variant thereof positioned at the hinge region between the antigen binding fragment (e.g., scFv or Fab) and an Fc domain subunit (or portion thereof) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of a VH of the antigen binding fragment (e.g., scFv or Fab), ii) the N-terminus of a VL of the antigen binding fragment (e.g., scFv or Fab), iii) the C-terminus of the Fc domain subunit (or portion thereof), iv) the C-terminus of a CL of the antigen binding fragment (Fab), and v) the N-terminus of the Fc domain subunit. In some embodiments, in the absence of binding of the antigen binding fragment (e.g., VHH, ligand, or receptor) to the target antigen, the activity (binding affinity to corresponding cytokine receptor or subunit thereof, and/or biological activity) of the cytokine or variant thereof positioned at the hinge region between the antigen binding fragment (e.g., VHH, ligand, or receptor) and an Fc domain subunit (or portion thereof) (see FIGS. 4A-4C) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding cytokine or variant thereof expressed at any of: i) the N-terminus of the antigen-binding fragment (e.g., VHH, ligand, or receptor), ii) the C-terminus of the Fc domain subunit (or portion thereof), and iii) the N-terminus of the Fc domain subunit; and in the presence of binding of the antigen binding fragment (e.g., VHH, ligand, or receptor) to the target antigen, the activity (binding affinity to corresponding cytokine receptor or subunit thereof, and/or biological activity) of the cytokine or variant thereof positioned at the hinge region between the antigen binding fragment (e.g., VHH, ligand, or receptor) and an Fc domain subunit (or portion thereof) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding cytokine or variant thereof expressed at any of: i) the N-terminus of the antigen-binding fragment (e.g., VHH, ligand, or receptor), ii) the C-terminus of the Fc domain subunit (or portion thereof), and iii) the N-terminus of the Fc domain subunit.

In some embodiments, the cytokine or variant thereof is a cytokine variant. In some embodiments, the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the activity (binding affinity to corresponding cytokine receptor or subunit thereof, and/or biological activity) of the cytokine variant in a free state is the same or similar (such as within about ±20% difference) of that of a corresponding wildtype cytokine in a free state.

In some embodiments, in the absence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine variant positioned at the hinge region of the antigen-binding polypeptide (such as positioned at the hinge region of a heavy chain of the antibody (e.g., full-length antibody), or positioned at the hinge region between an antigen binding fragment (e.g., ligand, receptor, VHH, scFv, or Fab) and an Fc domain subunit (or portion thereof)) is no more than about 80% (such as no more than about any of 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding wildtype cytokine (or a corresponding recombinant “wildtype” cytokine expressed in the same format but comprising wildtype subunits) positioned at the same region. For example, in some embodiments, the IL-12 variant comprises from N-terminus to C-terminus: variant p40 subunit—linker (e.g., any of SEQ ID NOs: 227-229)—wildtype p35 subunit, and the corresponding recombinant “wildtype” IL-12 comprises from N-terminus to C-terminus: wildtype p40 subunit—linker (e.g., any of SEQ ID NOs: 227-229)—wildtype p35 subunit. In some embodiments, the cytokine variant is an IL-2 variant, and the corresponding wildtype cytokine is a “wildtype” IL-2.

In some embodiments, in the presence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine variant positioned at the hinge region of the antigen-binding polypeptide (such as positioned at the hinge region a heavy chain of an antibody (e.g., full-length antibody), or positioned at the hinge region between an antigen binding fragment (e.g., ligand, receptor, VHH, scFv, or Fab) and an Fc domain subunit (or portion thereof)) is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, or more) of that of a corresponding wildtype cytokine (or a corresponding recombinant “wildtype” cytokine expressed in the same format but comprising wildtype subunits) positioned at the same region.

In some embodiments, in the absence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine variant positioned at the hinge region of the antigen-binding polypeptide (such as positioned at the hinge region of a heavy chain of an antibody (e.g., full-length antibody), or positioned at the hinge region between an antigen binding fragment (e.g., ligand, receptor, VHH, scFv, or Fab) and an Fc domain subunit (or portion thereof)) is no more than about 80% (such as no more than about any of 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding wildtype cytokine (or a corresponding recombinant “wildtype” cytokine in the same format but comprising wildtype subunits) positioned at the same region; and in the presence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen, the activity (binding affinity to corresponding cytokine receptor or subunit thereof, and/or biological activity) of the cytokine variant positioned at the hinge region of the antigen-binding polypeptide (such as positioned at the hinge region of a heavy chain of an antibody (e.g., full-length antibody), or positioned at the hinge region between an antigen binding fragment (e.g., ligand, receptor, VHH, scFv, or Fab) and an Fc domain subunit (or portion thereof)) is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, or more) of that of a corresponding wildtype cytokine (or a corresponding recombinant “wildtype” cytokine in the same format but comprising wildtype subunits) positioned at the same region.

Binding Affinity

Binding affinity of a molecule (e.g., cytokine moiety, or immunocytokine comprising a cytokine moiety) and its binding partner (e.g., cytokine receptor or subunits thereof) can be determined experimentally by any suitable ligand binding assays or antibody/antigen binding assays known in the art, e.g., Western blots, sandwich enzyme-linked immunosorbent assay (ELISA), Meso Scale Discovery (MSD) electrochemiluminescence, bead based multiplex immunoassays (MIA), RIA, Surface Plasma Resonance (SPR), ECL, IRMA, FACS, EIA, Biacore assay, Octet analysis, peptide scans, etc. For example, easy analysis is possible by using the cytokine or variant thereof, immunocytokine comprising the cytokine or variant thereof, or its corresponding receptor or subunits thereof marked with a variety of marker agents, as well as by using BiacoreX (made by Amersham Biosciences), which is an over-the-counter, measuring kit, or similar kit, according to the user's manual and experiment operation method attached with the kit.

In some embodiments, protein microarray is used for analyzing the interaction, function and activity of the cytokine moiety or immunocytokine described herein to its corresponding cytokine receptor, on a large scale. The protein chip has a support surface bound with a range of capture proteins (e.g., cytokine receptor or subunits thereof). Fluorescently labeled probe molecules (e.g., cytokine moiety or immunocytokine described herein) are then added to the array and upon interaction with the bound capture protein, a fluorescent signal is released and read by a laser scanner.

In some embodiments, the binding affinity of a cytokine moiety or immunocytokine described herein and its corresponding cytokine receptor (or subunit thereof) is measured using SPR (Biacore T-200). For example, anti-human antibody is coupled to the surface of a CM-5 sensor chip (e.g., using EDC/NHS chemistry). Then a human cytokine receptor-Fc fusion protein (e.g., IL-2Rα-Fc, IL-2Rβ-Fc, IL-2Rγ-Fc) is used as the captured ligand over this surface. Serial dilutions of immunocytokine comprising a cytokine moiety (e.g., IL-2 variant) are allowed to bind to the captured ligands (free state IL-2 variant serves as control), and the response units (RU) can be plotted against immunocytokine concentration to determine EC50 values, or plotted against time to monitor the binding and dissociation of immunocytokine to cytokine receptor-Fc in real time. Equilibrium dissociation constant (K_{D}) and dissociation rate constant can be determined by performing kinetic analysis using Biacore evaluation software. The binding affinity of each test immunocytokine to the cytokine receptor can be calculated as percentage relative to that of a corresponding free state cytokine moiety. In some embodiments, a cell line expressing a cytokine receptor (e.g., IL-2R) on the cell surface is incubated with an immunocytokine comprising a cytokine moiety (e.g., IL-2 variant) described herein, after incubation, the cells are washed, then an anti-IgG-conjugated with fluorescent protein (e.g., APC) is added to detect binding affinity of the immunocytokine to the cells, such as by FACS.

In some embodiments, the K_{D} of the binding between the cytokine or variant thereof in free state and its corresponding cytokine receptor (or subunits thereof) is about any of ≤10⁻⁵ M ≤10⁻⁶M, ≤10⁻⁷ M, ≤10⁻⁸ M, ≤10⁻⁹ M, ≤10¹⁰ M, ≤10⁻¹¹ M, or ≤10⁻¹ M. In some embodiments, in the absence of binding of the antigen-binding protein (e.g., antibody (e.g., full-length antibody), or ligand/receptor-hinge-Fc fusion protein) or antigen-binding fragment (e.g., ligand, receptor, VHH, scFv, or Fab) of the immunocytokine described herein to the target antigen, the K_{D} of the binding between the cytokine or variant thereof positioned at the hinge region of the antigen-binding polypeptide (such as positioned at the hinge region of a heavy chain of the antibody (e.g., full-length antibody), or positioned at the hinge region between an antigen binding fragment (e.g., ligand, receptor, VHH, scFv, or Fab) and an Fc domain subunit (or portion thereof)) and its corresponding cytokine receptor (or subunits thereof) is undetectable (e.g., no binding), or the K_{D} is higher than (i.e., binds weaker than) that in the presence of binding of the antigen-binding protein (e.g., antibody (e.g., full-length antibody), or ligand/receptor-hinge-Fc fusion protein) or antigen binding fragment (e.g., ligand, receptor, VHH, scFv, or Fab) of the immunocytokine described herein to the target antigen.

Biological Activity

Various methods for determining the biological activities (or bioactivities) of cytokines or variants thereof, or immunocytokines described herein are described in the art, such as bioassays. Any cytokine assays known in the art can be adapted to test bioactivities of cytokine moieties or immunocytokines described herein.

A bioassay focuses on biological activity of cytokines and using it as a read out. In a bioassay, the activity of a sample is tested on a sensitive cell line (e.g., primary cell cultures or in vitro adapted cell lines that are dependent and/or responsive to the test sample) and the results of this activity (e.g., cellular proliferation) are compared to a standard cytokine preparation. Other aspects of biological activity of cytokines include induction of further cytokine secretion, induction of killing, antiviral activity, degranulation, cytotoxicity, chemotaxis, and promotion of colony formation. In vitro assays to measure all of these activities are available. See, e.g., “Cytokine Bioassays” of Bioassays—BestProtocols®, from eBioscience® (http://tools.thermofisher.com/content/sfs/manuals/cytokine-bioassays.pdf), the content of which is incorporated herein by reference in its entirety.

For example, in a cytokine-induced proliferation assay, samples (e.g., IL-2 moiety or IL-2 immunocytokine) and standard (e.g., IL-2 in free state) are diluted via serial dilution in an assay plate filled with culture medium, indicator cells (e.g., CTLL-2, or PBMC stimulated with anti-CD3 Ab) are washed and resuspend in culture medium then added into each well. The cells are incubated for sufficient time (e.g., 24 hours or longer) at 37° C., 5% C02 in a humidified incubator.

Then cell viability test agents (e.g., resazurin, MTT assay agents) can be added to the plate and allow for sufficient incubation, then read with spectrophotometer. The EC50 values (concentration of test sample required to exhibit 50% of maximal response) for cell proliferation can then be obtained from non-linear regression analysis of dose-response curves. Cell number can also be counted under microscope, and compare to that treated with standard or control. For another example, in a cytokine-induced cytokine production assay, samples (e.g., IL-12 or IL-23 moiety, or IL-12 or IL-23 immunocytokine) and standard (e.g., IL-12 or IL-23 in free state) are diluted via serial dilution in an assay plate filled with culture medium, indicator cells (e.g., splenocytes, activated CD4+ T cells, or activated CD8+ T cells) are washed and resuspend in culture medium then added into each well. Cells are incubated for sufficient time (e.g., 24-48 hours) at 37° C., 5% C02 in a humidified incubator, then supernatants are harvest for determination of cytokine expression by ELISA, following ELISA protocol for target cytokine of interest (e.g., IFN-γ). For another example, in a cytokine-induced cell surface marker expression assay, samples (e.g., IFN-γ moiety, or IFN-γ immunocytokine) and standard (e.g., IFN-γ in free state) are diluted via serial dilution in an assay plate filled with culture medium, indicator cells (e.g., HEK-Blue™ IFN-γ cells) are washed and resuspend in culture medium then added into each well. Cells are incubated for sufficient time (e.g., 24-48 hours) at 37° C., 5% C02 in a humidified incubator, then cell surface expression of biomarker (e.g., PD-L1) can be detected (e.g., using anti-human PD-L1 APC-conjugated antibody) and measured by ELISA or FACS. Also see Examples 1, 5, and 11 for exemplary methods.

Bioactivities of cytokine moieties or immunocytokines described herein can also be reflected by in vivo or ex vivo experiments, for example, by measuring the proliferation of indicator cells (e.g., after administering IL-2 moieties or IL-2 immunocytokines, the proliferation of CD8+ cells, NK cells, or Tregs); by measuring the induction or inhibition of cytokine secretion; by measuring tumor volume reduction in tumor xenograft mice after injecting the test cytokine moieties or immunocytokines described herein; or by measuring autoimmune score.

Cell signaling assays can also be used to test bioactivities of cytokine moieties or immunocytokines described herein. Various cell signaling assay kits are commercially available, for example, to detect analytes produced during enzymatic reactions involved in signaling such as ADP, AMP, UDP, GDP, and growth factors, or phosphatase assays, to quantify both total and phosphorylated forms of signaling proteins. For example, after incubating the cells with cytokine moieties or immunocytokines described herein, to determine whether a particular kinase is active, the cell lysate is exposed to a known substrate for the enzyme in the presence of radioactive phosphate. The products are separated by electrophoresis (with or without immunoprecipitation), then the gel is exposed to x-ray film to determine whether the proteins incorporated the isotope. In some embodiments, the bioactivities of cytokine moieties or immunocytokines described herein on cells are measured by immunohistochemistry to locate signaling proteins. For example, antibodies to the signal proteins themselves or signal proteins in their activated state can be used. These antibodies have recognition epitopes that include the phosphate or other activating conformation. In some embodiments, movement of specific signaling proteins (e.g., nuclear translocation of signaling molecules) can be tracked by incorporating a fluorescent protein gene, e.g., green fluorescent protein (GFP), into genetic vectors encoding the protein to be studied. In some embodiments, bioactivities of cytokine moieties or immunocytokines described herein on cells are tested by western blots. For example, all tyrosine-phosphorylated proteins (or other phosphorylated amino acids, e.g., serine or threonine) can be detected with an anti-phosphotyrosine antibody (or antibodies against other phosphorylated amino acids) on a Western blot of cell lysates obtained after stimulation in a temporal sequence. In some embodiments, the bioactivities of cytokine moieties or immunocytokines described herein on cells can be measured by immunoprecipitation. For example, primary antibodies to a specific signaling protein or all tyrosine-phosphorylated proteins are cross-linked to the beads. The cells after incubating with cytokine moieties or immunocytokines described herein are lysed in buffer containing protease inhibitors and then incubated with the antibody-coated beads. The proteins are separated by using SDS electrophoresis, and then the proteins are identified by using the procedures described for Western blots. In some embodiments, glutathione S-transferase (GST) binding, or “pull-down” assay, can also be used, which determines direct protein-protein (e.g., signaling protein) interactions. Cell-based signal transduction assays can also be used. Briefly, a reporter cell line (e.g., HEK-Blue™) stably expressing the corresponding receptor of the test cytokine moiety or immunocytokine, corresponding signaling factors of the cytokine signaling pathway (e.g., STAT, JAK), and cytokine signaling pathway-inducible reporter (e.g., fluorescent protein, or secreted embryonic alkaline phosphatase) can be cultured in the presence of the test cytokine moiety or immunocytokine at 37° C. in a C02 incubator for sufficient time (e.g., 24-48 hours), then the reporter can be detected, such as using microscopy or FACS for fluorescent protein, or to detect secreted embryonic alkaline phosphatase in cell culture medium using colorimetric enzyme assay for alkaline phosphatase activity (e.g., QUANTI-Blue™). Also see Examples 1, 4, 7, 9, 10, and 12 for exemplary methods.

Using IL-2 as an example, STAT5 and ERK1/2 signaling can be measured to reflect IL-2 moiety or immunocytokine bioactivity, for example, by measuring phosphorylation of STAT5 and ERK1/2 using any suitable method known in the art. For example, STAT5 and ERK1/2 phosphorylation can be measured using antibodies specific for the phosphorylated version of these molecules in combination with flow cytometry analysis. For example, freshly isolated PBMCs are incubated at 37° C. with IL-2 or variant thereof, or IL-2 immunocytokine. After incubation, cells are immediately fixed (e.g., with Cytofix buffer) to preserve the phosphorylation status and permeabilized (e.g., with Phosflow Perm buffer III). The cells are stained with fluorophore-labeled antibodies against phosphorylated STAT5 or ERK1/2, and analyzed by flow cytometry. Alternatively, test samples (e.g., IL-2 cytokine moieties or IL-2 immunocytokines described herein) can be injected i.p. into mice, then total splenocytes can be isolated, immediately fixed (e.g., Phosphoflow™ Lyse/Fix buffer), washed with ice cold PBS, stained using anti-CD4 and anti-CD25 antibodies, and then permeabilized (e.g., PhosFlow Perm Buffer III). Cells are then washed with ice-cold FACS buffer, stained with anti-FoxP3, washed with ice-cold FACS buffer, and stained with fluorophore-labeled anti-phospho-STAT5 at room temperature. Cells are washed with FACS buffer, then data can be acquired on a FACS cytometer and analyzed. PI 3-kinase signaling can be measured using any suitable method known in the art to reflect IL-2 bioactivity, too. For example, PI 3-kinase signaling can be measured using antibodies that are specific for phospho-S6 ribosomal protein in conjunction with flow cytometry analysis.

In some embodiments, the cytokine moieties or immunocytokines described herein is capable of activating an immune cell, such as inducing test cytokine (e.g., IL-2 moiety or IL-2 immunocytokine described herein) dependent immune cell (e.g., PBMC, NK cell, CD8+ T cell, Th17 cell) proliferation, differentiation, and/or activation, cytokine secretion, activating signaling transduction (e.g., inducing STAT5 phosphorylation, ERK1/2 phosphorylation, or stimulating PI 3-kinase signaling), and/or inducing immune cells to kill tumor cells or infected cells. In some embodiments, the cytokine moieties or immunocytokines described herein is capable of inhibiting an immune cell, such as inhibiting cytokine (e.g., pro-inflammatory cytokine) production, antigen presentation, or MHC molecule expression from the immune cell, or inhibiting or ameliorating signaling transduction. In some embodiments, the immune cell is selected from the group consisting of a monocyte, a dendritic cell, a macrophage, a B cell, a killer T cell (T_{c}, cytotoxic T lymphocyte, or CTL), a helper T cell (Th), a regulatory T cells (Treg), a γδ T cell, a natural killer T (NKT) cell, and a natural killer (NK) cell.

In some embodiments, the activity in activating/inhibiting an immune cell of the cytokine variant in a free state is the same or similar (such as within about ±20% difference) of that of a corresponding wildtype cytokine in a free state. In some embodiments, the cytokine variant comprises a mutation or a modification (e.g., post-translational modification), which reduces its activity in activating/inhibiting an immune cell compared to the wildtype cytokine (e.g., no more than about any of 80%, 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.10%, or 0% of the bioactivity of wildtype cytokine), when in a free state or in the absence of target antigen-antibody binding of the immunocytokine described herein. In some embodiments, in the presence of target antigen-antibody binding of the immunocytokine described herein, the activity in activating/inhibiting an immune cell of the cytokine variant is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, or 200%) of that of a corresponding wildtype cytokine.

### Antigen-Binding Protein, Antigen-Binding Polypeptide, Antibody, and Antigen-Binding Fragment

A “parental” antigen-binding protein described herein is an antigen-binding protein (e.g., antibody, antibody fragment, or ligand/receptor-hinge-Fc fusion protein) that can specifically recognize a target antigen and serve as backbone for constructing the immunocytokines described herein. In some embodiments, the antigen-binding protein (e.g., parental antigen-binding protein) comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, Fab, VG, or VL), an optional linker (such as any peptide linkers described herein), a hinge region, and an Fc domain subunit or portion thereof. In some embodiments, the antigen-binding protein (e.g., parental antigen-binding protein) comprises (e.g., consists essentially of, or consists of) homodimeric antigen-binding polypeptides, i.e., two identical antigen-binding polypeptides. For example, in some embodiments, the parental antigen-binding protein comprises (e.g., consists essentially of, or consists of) two antigen-binding polypeptides each comprising from N′ to C′: an antigen-binding fragment (e.g., a ligand (such as PD-L2) or portion thereof, a receptor (such as PD-1) or portion thereof, VHH, scFv, VG, or VL), an optional linker, a hinge region, and an Fc domain subunit or portion thereof. In some embodiments, the antigen-binding protein (e.g., parental antigen-binding protein) comprises (e.g., consists essentially of, or consists of) heterodimeric antigen-binding polypeptides, i.e., two antigen-binding polypeptides different in one or more of antigen-binding fragment (e.g., ligand, receptor, VHH, scFv, VG, or VL), optional linker, hinge region, and Fc domain subunit or portion thereof. For example, in some embodiments, the two antigen-binding polypeptides comprise different antigen-binding fragments (e.g., bind to the same target epitope or different target epitopes). In some embodiments, the two antigen-binding polypeptides comprise different hinge regions and/or different Fc domain subunits or portion thereof, e.g., for heterodimeric pairing purpose. For example, in some embodiments, one antigen-binding polypeptide comprises a hinge region comprising the sequence of SEQ ID NO: 41, and the pairing antigen-binding polypeptide comprises a hinge region comprising the sequence of SEQ ID NO: 42. In some embodiments, one antigen-binding polypeptide comprises a hinge region comprising the sequence of SEQ ID NO: 51, and the pairing antigen-binding polypeptide comprises a hinge region comprising the sequence of SEQ ID NO: 52. In some embodiments, one antigen-binding polypeptide comprises an Fc domain subunit comprising the sequence of SEQ ID NO: 61, and the pairing antigen-binding polypeptide comprises an Fc domain subunit comprising the sequence of SEQ ID NO: 62. In some embodiments, the parental antigen-binding protein (e.g., antibody such as full-length antibody, antigen-binding fragment-hinge-Fc fusion protein such as ligand/receptor-hinge-Fc fusion protein) comprises two or more antigen-binding fragments (e.g., ligand, receptor, VHH, scFv, Fab, VH, or VL). In some embodiments, one antigen-binding polypeptide comprises two or more antigen-binding fragments (e.g., two or more ligands and/or receptors fused in tandem). In some embodiments, the two or more antigen-binding fragments are the same. In some embodiments, the two or more antigen-binding fragments are different, e.g., one is Fab, the other is scFv; one is a ligand, the other is a receptor. In some embodiments, the parental antigen-binding protein (e.g., antibody such as full-length antibody, antigen-binding fragment-hinge-Fc fusion protein such as ligand/receptor-hinge-Fc fusion protein) or two or more antigen-binding fragments specifically recognize two or more target epitopes (on the same target antigen or different target antigens). In some embodiments, the parental antigen-binding protein or two or more antigen-binding fragments specifically recognize one target epitope. In some embodiments, the parental antigen-binding protein, antigen-binding polypeptide, or antigen-binding domain is monospecific and multivalent (e.g., bivalent). In some embodiments, the parental antigen-binding protein, antigen-binding polypeptide, or antigen-binding domain is multispecific (e.g., bispecific) and multivalent (e.g., bivalent). In some embodiments, the parental antigen-binding protein or antigen-binding domain is monospecific and monovalent, i.e., the cytokine moiety is positioned at the hinge region between an antigen-binding fragment (e.g., Fab, scFv, VHH, ligand, or receptor) and one subunit of the Fc domain (or portion thereof), and the other subunit of the Fc domain (or portion thereof) is not fused with another antigen-binding fragment; or the cytokine moiety is positioned at the hinge region between one antigen-binding fragment (e.g., Fab, scFv, VHH, ligand, or receptor) and a monomeric Fc subunit (or portion thereof).

It is to be understood that for the present invention, reference to an “antigen-binding protein,” “antigen-binding fragment-hinge-Fc fusion protein,” “ligand-hinge-Fc fusion protein,” or “receptor-hinge-Fc fusion protein” includes: i) an antigen-binding protein backbone (e.g., antibody such as full-length antibody), an antigen-binding fragment-hinge-Fc fusion protein backbone, or a ligand/receptor-hinge-Fc fusion protein backbone (see, e.g., FIGS. 2A-4C light grey portions); or ii) parental antigen-binding protein, parental antigen-binding fragment-hinge-Fc fusion protein, or parental ligand/receptor-hinge-Fc fusion protein whose hinge region has a cytokine moiety positioned therein (see, e.g., FIGS. 2A-4C). It is also to be understood that for the present invention, reference to an “antigen-binding fragment-hinge-Fc fusion polypeptide,” “ligand-hinge-Fc fusion polypeptide,” or “receptor-hinge-Fc fusion polypeptide” includes: i) an antigen-binding fragment-hinge-Fc fusion polypeptide backbone (e.g., antibody heavy chain) or ligand/receptor-hinge-Fc fusion polypeptide backbone (see, e.g., light grey portions of an Fc-containing polypeptide chain in FIGS. 2A-4C), or ii) parental antigen-binding fragment-hinge-Fc fusion polypeptide or ligand/receptor-hinge-Fc fusion polypeptide whose hinge region has a cytokine moiety positioned therein (see, e.g., the cytokine-Fc-containing polypeptide chain in FIGS. 2A-4C).

In some embodiments, the “parental” antigen-binding protein is an antigen-binding fragment-optional linker-hinge-Fc fusion protein comprising (e.g., consisting essentially of, or consisting of) two antigen-binding fragment-optional linker-hinge-Fc polypeptides. In some embodiments, the “parental” antigen-binding protein is a ligand or portion thereof-optional linker-hinge-Fc fusion protein comprising (e.g., consisting essentially of, or consisting of) two ligand or portion thereof-optional linker-hinge-Fc polypeptides (see FIGS. 4A-4C light grey portions). In some embodiments, the “parental” antigen-binding protein is a receptor or portion thereof-optional linker-hinge-Fc fusion protein comprising (e.g., consisting essentially of, or consisting of) two receptor or portion thereof-optional linker-hinge-Fc polypeptides. In some embodiments, the ligand or receptor portion thereof is extracellular domain of the ligand or receptor. In some embodiments, the parental antigen-binding protein is a PD-L2 extracellular domain-optional linker-hinge-Fc fusion protein. The one or more cytokine moieties (or cytokine subunits) are positioned at the hinge region of one or both of the parental antigen-binding fragment-hinge-Fc polypeptides (see, e.g., dark grey or pattern filled ovals in FIGS. 4A-4C). In some embodiments, the antigen-binding fragment can be any ligand or receptor (or portion thereof, such as extracellular domain) known in the art or derived from any suitable ligands or receptors, as long as binding of the cytokine moiety to its cytokine receptor is reduced in the absence of target antigen(s) binding of the antigen-binding fragment(s), for example, without target antigen (e.g., corresponding receptor or ligand) binding, reducing the activity (binding affinity to cytokine receptor and/or biological activity) of the cytokine or variant thereof positioned at the hinge region to be no more than about 70% of that of a corresponding cytokine or variant thereof in a free state. For example, the ligand or receptor can be selected from any ligand or receptor in below “Cell surface ligand or receptor” subsection.

In some embodiments, the antigen-binding protein is an antibody or antigen-binding fragments (e.g., antibody fragments, such as Fab, scFv, VHH etc.) thereof. A “parental” antibody or antigen-binding fragment described herein is an antibody or antigen-binding fragment that served as backbone for constructing the immunocytokine. In some embodiments, the parental antibody of the immunocytokine described herein is a full-length antibody. See, e.g., filled light grey portions in FIGS. 1A-2D. In some embodiments, the parental antibody of the immunocytokine described herein is an antigen-binding fragment. See, e.g., light grey portions in FIGS. 3A-3C. For example, in some embodiments, the parental antibody comprises two scFvs each fused at the N-terminus of a subunit of an Fc domain via a hinge or portion thereof (see FIG. 3A light grey portions). In some embodiments, the parental antibody comprises two scFvs each fused at the N-terminus of a subunit of an Fc domain portion (e.g., CH2) via a hinge or portion thereof. In some embodiments, the parental antibody comprises two Fabs each fused at the N-terminus of a subunit of an Fc domain portion (e.g., CH2) via a hinge or portion thereof. In some embodiments, the parental antibody comprises one scFv fused at the N-terminus of one subunit of an Fc domain via a hinge or portion thereof, and one Fab fused at the N-terminus of the other subunit of the Fc domain via a hinge or portion thereof (see FIGS. 3B-3C light grey portions). In some embodiments, the parental antibody comprises one scFv fused at the N-terminus of one subunit of an Fc domain portion (e.g., CH2) via a hinge or portion thereof, and one Fab fused at the N-terminus of the other subunit of the Fc domain portion (e.g., CH2) via a hinge or portion thereof. In some embodiments, the parental antibody comprises a first VHH fused at the N-terminus of one subunit of an Fc domain or portion thereof via a hinge or portion thereof, and a second VHH fused at the N-terminus of the other subunit of the Fc domain or portion thereof via a hinge or portion thereof (see FIGS. 4A-4C light grey portions and replace “ligand” or “receptor” with VHH). The one or more cytokine moieties (or cytokine subunits) are positioned at the hinge region of any of the parental antibody (e.g., full-length antibody) or antigen-binding fragment (see, e.g., dark grey or pattern filled ovals in FIGS. 2A-4C).

The antigen-binding fragment can be of any format known in the art or derived from any suitable antibodies, as long as binding of the cytokine moiety to its cytokine receptor is reduced in the absence of target antigen(s) binding of the antigen-binding fragment(s), for example, without target antigen binding, reducing the activity (binding affinity to cytokine receptor and/or biological activity) of the cytokine or variant thereof positioned at the hinge region to be no more than about 70% of that of a corresponding cytokine or variant thereof in a free state. For example, the antigen-binding fragment can be selected from an scFv, a VH, a VL, an scFv-scFv, an Fv, a Fab, a Fab′, a (Fab′)2, a minibody, a diabody, a domain antibody variant (dAb), a single domain antibody (sdAb) such as a camelid antibody (VHH) or a VNAR, a fibronectin 3 domain variant, an ankyrin repeat variant, and other antigen-specific binding domains derived from other protein scaffolds. In some embodiments, the antigen-binding fragment is an scFv. In some embodiments, the antigen-binding fragment is a Fab. In some embodiments, the antigen-binding fragment is formed by a VH from a first polypeptide chain and a VL from a second polypeptide chain. In some embodiments, the antigen-binding fragment is human. In some embodiments, the antigen-binding fragment is humanized. In some embodiments, the antigen-binding fragment is chimeric. In some embodiments, the antigen-binding fragment is derived from a monoclonal antibody of mouse, rat, monkey, or rabbit, etc.

In some embodiments, two or more antigen-binding fragments are connected in tandem via optional linker(s). For example, in some embodiments, the immunocytokine comprises from N′ to C′: Fab1—optional linker1—Fab2—optional linker2—(optional hinge or portion thereof-cytokine moiety—optional hinge or portion thereof)—Fc subunit. For example, CH1 or CL of Fab1 is linked to VH or VL of Fab2 via the optional linker 1. In some embodiments, the immunocytokine comprises from N′ to C′: scFv1 (or sdAb1)—optional linker1—scFv2 (or sdAb2)—optional linker 2—(optional hinge or portion thereof—cytokine moiety—optional hinge or portion thereof)—Fc subunit. In some embodiments, the immunocytokine comprises from N′ to C′: ligand1—optional linker1—ligand2—optional linker 2—(optional hinge or portion thereof-cytokine moiety—optional hinge or portion thereof)—Fc subunit. The cytokine moiety in parenthesis can be absent for the other pairing immunocytokine chain. For example, the immunocytokine can comprise a first polypeptide chain comprising from N′ to C′: scFv1 (or sdAb1)—optional linker1—scFv2 (or sdAb2)—optional linker2—cytokine moiety—hinge or portion thereof—Fc subunit1; and a second polypeptide chain from N′ to C′: scFv3 (or sdAb3)-optional linker3—scFv4 (or sdAb4)—optional linker4—hinge or portion thereof—Fc subunit2.

Binding affinity of an antigen-binding protein (e.g., antibody or ligand/receptor-hinge-Fc fusion protein) or antigen-binding fragment (e.g., scFv, Fab, VHH, ligand, or receptor) and its target antigen can be determined experimentally by any suitable antibody/antigen binding assays or other protein binding assays (e.g., ligand-receptor binding) known in the art, e.g., Western blots, ELISA, MSD electrochemiluminescence, bead based MIA, RIA, SPR, ECL, IRMA, EIA, Biacore assay, Octet analysis, peptide scans, FACS, etc. Also see “binding affinity” subsection above for exemplary methods. In some embodiments, the Kd of the binding between the antibody or antigen-binding fragment and its target antigen is about any of ≤10⁻⁵ M, ≤10⁻⁶ M, ≤10⁻⁷ M, ≤10⁻¹ M, ≤10⁻⁹ M, ≤10¹⁰ M, ≤10⁻¹¹ M, or 10⁻⁵ M.

Amino acid sequence variants of an antigen-binding protein (e.g., antibody or ligand/receptor-hinge-Fc fusion protein) or antigen-binding fragment may be prepared by introducing appropriate modifications into the nucleic acid sequence encoding the antigen-binding protein or antigen-binding fragment, or by peptide synthesis. Such modifications include, for example, deletions from, and/or insertions into and/or substitutions of residues within the amino acid sequences of the antibody or antigen-binding fragment. Any combination of deletion, insertion, and substitution can be made to arrive at the final construct, provided that the final construct possesses the desired characteristics, e.g., antigen-binding.

In some embodiments, the antigen-binding protein (e.g., antibody or ligand/receptor-hinge-Fc fusion protein) or antigen-binding fragment (e.g., scFv, Fab, VHH, ligand, or receptor) has one or more amino acid substitutions. Sites of interest for substitutional mutagenesis include the HVRs (or CDRs) and FRs of antibodies or antigen-binding fragments. Conservative substitutions are shown in Table B under the heading of “Preferred substitutions.” More substantial changes are provided in Table B under the heading of “exemplary substitutions,” and as further described below in reference to amino acid side chain classes. Amino acid substitutions may be introduced into an antigen-binding fragment of interest and the products screened for a desired activity, e.g., retained/improved antigen binding, decreased immunogenicity.

TABLE B
Amino acid substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes for another class.

One type of substitutional variant involves substituting one or more HVR residues of a parent antibody. Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

In some embodiments, substitutions, insertions, or deletions may occur within one or more HVRs so long as such alterations do not substantially reduce the ability of the antibody or antigen-binding domain to bind antigen. For example, conservative alterations (e.g., conservative substitutions as provided herein) that do not substantially reduce binding affinity may be made in HVRs. Such alterations may be outside of HVR “hotspots” or CDRs.

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

### Target Antigens

In some embodiments, the immunocytokine described herein comprises an antigen-binding protein specifically recognizing a target antigen. In some embodiments, the antigen-binding protein comprises an antigen-binding polypeptide comprising from N′ to C′: an antigen-binding fragment (e.g., ligand, receptor, VHH, scFv, or Fab), a hinge region, and an Fc domain subunit or portion thereof “Target antigen” or “target epitope” used herein can refer to any protein or polypeptide that can be specifically recognized by the antigen-binding protein, antigen-binding polypeptide, or antigen-binding fragment described herein, such as tumor antigen or epitope, pathogen antigen or epitope, antigen or epitope involved in autoimmune diseases, allergy, and/or graft rejection, ligand or receptor or portion thereof (e.g., extracellular domain of a ligand/receptor), immune cell surface antigen or epitope, etc. In some embodiments, the antigen-binding protein is monovalent and monospecific. In some embodiments, the antigen-binding protein is multivalent (e.g., bivalent) and monospecific. In some embodiments, the antigen-binding protein is multivalent (e.g., bivalent) and multispecific (e.g., bispecific). The valency and specificity of the antigen-binding protein herein is referring to valency and specificity of the antigen-binding fragment(s) (e.g., ligand, receptor, VHH, scFv, or Fab) of the immunocytokine, not including valency or specificity of the cytokine or variant thereof.

In some embodiments, the antigen-binding fragment is a ligand (e.g., CD155, PD-L1 or PD-L2 extracellular domain) that specifically recognizes a cell surface receptor (e.g., PD-1). In some embodiments, the antigen-binding fragment is a receptor (e.g., TIGIT or PD-1 extracellular domain) that specifically recognizes a ligand (e.g., CD155, PD-L1 or PD-L2). In some embodiments, the antigen-binding protein comprises two or more (such as 2) antigen-binding polypeptides each comprising from N′ to C′: an antigen-binding fragment, a hinge region, and an Fc domain subunit or portion thereof. In some embodiments, the antigen-binding polypeptide comprises from N′ to C′: two or more antigen-binding fragments (e.g., ligand or receptor), e.g., fused in tandem, a hinge region, and an Fc domain subunit or portion thereof. The two or more antigen-binding fragments can be the same or different, can specifically recognize the same target epitope or different target epitopes (on the same target antigen or different target antigens). For example, in some embodiments, one antigen-binding fragment is derived from a first ligand (e.g., extracellular domain of first ligand) that specifically recognizes a first receptor or epitope thereof (e.g., extracellular domain of first receptor), another antigen-binding fragment is derived from a second receptor (e.g., extracellular domain of second receptor) that specifically recognizes a second ligand or epitope thereof (e.g., extracellular domain of second ligand). In some embodiments, one antigen-binding fragment is derived from a first ligand (e.g., extracellular domain of first ligand) that specifically recognizes a first receptor or epitope thereof (e.g., extracellular domain of first receptor), another antigen-binding fragment is derived from a second ligand (e.g., extracellular domain of second ligand) that specifically recognizes a second receptor or epitope thereof (e.g., extracellular domain of second receptor). The first and second receptors (or epitopes thereof) can be the same or different. The first and second ligands (or epitopes thereof) can be the same or different. In some embodiments, the first and second antigen-binding fragments (ligand or receptor) specifically recognize the same epitope of a target antigen (corresponding receptor or ligand, respectively), i.e., the immunocytokine is bivalent and monospecific. In some embodiments, the first and second antigen-binding fragments (ligand or receptor) specifically recognize different epitopes of the same target antigen (corresponding receptor or ligand, respectively), i.e., the immunocytokine is bivalent and bispecific. In some embodiments, the first and second antigen-binding fragments (ligand or receptor) specifically recognize different target antigens (e.g., different epitopes of different target antigens, such as corresponding receptor or ligand, respectively), i.e., the immunocytokine is bivalent and bispecific.

In some embodiments, the antigen-binding protein is an antibody or portion thereof. Thus in some embodiments, the immunocytokine described herein comprises an antibody (e.g., full-length antibody, heavy chain only antibody, or antigen-binding fragment fused to an Fc domain subunit or portion thereof) specifically recognizing a target antigen. In some embodiments, the antibody comprises two or more antigen-binding fragments (e.g., VHH, scFv, or Fab). The two antigen-binding domains (Fabs) of the full-length antibody, or the two or more antigen-binding fragments (e.g., VHH, scFv, or Fab) of the antibody, can be the same or different, can specifically recognize the same target epitope or different target epitopes (on the same target antigen or different target antigens). In some embodiments, the immunocytokine described herein comprises one or more antigen-binding domains (e.g., scFv or Fab) that specifically recognize one or more target epitopes (can be the same or different, can be on the same target antigen or different target antigens). In some embodiments, the immunocytokine comprises a first antigen-binding domain (e.g., VHH, scFv, or Fab) specifically recognizing a first target epitope, and a second antigen-binding domain (e.g., VHH, scFv, or Fab) specifically recognizing a second target epitope. In some embodiments, the first and the second target epitopes are the same. In some embodiments, the first and the second target epitopes are different (e.g., different epitopes on the same target antigen, or different epitopes of different target antigens). In some embodiments, the first and second antigen-binding domains specifically recognize the same epitope of a target antigen, i.e., the immunocytokine is bivalent and monospecific. In some embodiments, the first and second antigen-binding domains specifically recognize different epitopes of the same target antigen, i.e., the immunocytokine is bivalent and bispecific. In some embodiments, the first and second antigen-binding domains specifically recognize different target antigens (e.g., different epitopes of different target antigens), i.e., the immunocytokine is bivalent and bispecific. In some embodiments, the first target antigen is an immune cell surface antigen, and the second target antigen is a tumor antigen.

In some embodiments, the target antigen is a cell surface molecule (e.g., extracellular domain of a receptor/ligand). In some embodiments, the target antigen acts as a cell surface marker on a target cell (e.g., tumor cell, immune cell) associated with a special disease state. The target antigens (e.g., tumor antigen, extracellular domain of a receptor/ligand) specifically recognized by the antigen-binding domain may be antigens on a single diseased cell or antigens that are expressed on different cells that each contribute to the disease. The target antigens specifically recognized by the antigen-binding domain(s) may be directly or indirectly involved in the diseases.

Tumor Antigen

In some embodiments, the target antigen or epitope is a tumor antigen or epitope.

Tumor antigens are proteins that are produced by tumor cells that can elicit an immune response, particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), β-human chorionic gonadotropin, alpha-fetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-1, IGF-II, IGF-I receptor, and mesothelin. In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens is onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells. Non-limiting examples of TSA or TAA antigens include the following: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4 (TPBG), 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

In some embodiments, the tumor antigen is selected from the group consisting of FIXa, FX, DLL3, DLL4, Ang-2, Nectin-4, FOLRa, GPNMB, CD56 (NCAM), TACSTD2 (TROP-2), tissue factor, ENPP3, P-cadherin, STEAPI, CEACAM5, Mucin 1 (Sialoglycotope CA6), Guanylyl cyclase C (GCC), SLC44A4, LIV1 (ZIP6), NaPi2b, SLITRK6, SC-16, fibronectin, extra-domain B (EDB), Endothelium receptor ETB, ROBO4, Collagen IV, Periostin, Tenascin c, CD74, CD98, Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), RORI, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2 (CD309), LewisY, CD24, platelet-derived growth factor receptor-beta (PDGFR-beta), SSEA-4, CD20, Folate receptor alpha, ERBB2 (HER2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, CLDN18.2, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTi, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIBI, BORIS, SART3, PAX5, OY-TESI, LCK, AKAP-4, SSX2, RAGE-1, human telomerase reverse transcriptase, RU1, RU2, intestinal carboxyl esterase, mut hsp70-2, CD79a, CD79b, CD72, LAIRi, FCAR, LILRA2, CD300LF, CLEC12A, BST2, EMR2, LY75, GPC3, FCRL5, and IGLL1. In some embodiments, the tumor antigen is selected from the group consisting of BCMA, EphA2, HER2, GD2, Glypican-3, 5T4, 8H9, Wv06 integrin, B7-H3, B7-H6, CAIX, CA9, CD19, CD20, CD22, CD30, CD33, CD38, CD44, CD44v6, CD44v7/8, CD70 (TNFSF7), CD123, CD138, CD171, CEA, CSPG4, EGFR, EGFRvIII, EGP2, EGP40, EpCAM, ERBB3, ERBB4, ErbB3/4, FAP, FAR, FBP, fetal AchR, Folate Receptor a, GD2, GD3, HLA-A1 MAGE A1, HLA-A2, IL11Ra, IL13Ra2, KDR, Lewis-Y, MCSP, Mesothelin, Muci, Muc16, NCAM, NKG2D ligands, NY-ESO-I, PRAME, PSCA, PSCI, PSMA, RORI, SURVIVIN, TAG72, TEM1, TEM8, VEGFR2, carcinoembryonic antigen, and HMW-MAA. Also see exemplary tumor antigens described in Shim H. (Biomolecules. 2020 March; 10(3): 360), and Diamantis N. and Banerji U. Br J Cancer. 2016; 114(4): 362-367, the contents of which are incorporated herein by reference in their entirety.

In some embodiments, the tumor antigen is HER2. In some embodiments, the antibody or antigen-binding domain specifically recognizing HER2 is derived from trastuzumab (e.g., Herceptin®), pertuzumab (e.g., Perjeta®), margetuximab, or 7C2. In some embodiments, the antibody or antigen-binding domain specifically recognizing HER2 comprises heavy chain CDRs, light chain CDRs, or all 6 CDRs of any of trastuzumab, pertuzumab, margetuximab, or 7C2. In some embodiments, the antibody or antigen-binding domain specifically recognizing HER2 comprises VH and/or VL of trastuzumab, pertuzumab, margetuximab, or 7C2. In some embodiments, the immunocytokine comprises a parental anti-HER2 antibody (e.g., full-length antibody). In some embodiments, anti-HER2 antibody or antigen binding fragment thereof comprises: i) a VH-CDR1 comprising the sequence of SEQ ID NO: 188; ii) a VH-CDR2 comprising the sequence of SEQ ID NO: 189; iii) a VH-CDR3 comprising the sequence of SEQ ID NO: 190; iv) a VL-CDR1 comprising the sequence of SEQ ID NO: 191; v) a VL-CDR2 comprising the sequence of SEQ ID NO: 192; and vi) a VL-CDR3 comprising the sequence of SEQ ID NO: 193. In some embodiments, the anti-HER2 antibody or antigen binding fragment thereof comprises a VH comprising the sequence of SEQ ID NO: 150 and a VL comprising the sequence of SEQ ID NO: 151. In some embodiments, the anti-HER2 antibody (e.g., parental full-length antibody) comprises two heavy chains each comprising the sequence of SEQ ID NO: 152 or 153, and two light chains each comprising the sequence of SEQ ID NO: 154. In some embodiments, the anti-HER2 antibody (e.g., parental antibody) is a homodimeric full-length antibody (i.e., comprising two identical heavy chains and two identical light chains). In some embodiments, the anti-HER2 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

Pathogen Antigen

In some embodiments, the target antigen or epitope is a pathogen antigen or epitope, such as a fungal, viral, bacterial, protozoal or other parasitic antigen or epitope.

In some embodiments, the fungal antigen is from Aspergillus or Candida. Fungal antigens for use with compositions and methods of the invention include, but are not limited to, e.g., candida fungal antigen components; aspergillus fungal antigens; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Bacterial antigens for use with the immunocytokine disclosed herein include, but are not limited to, e.g., bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: haemophilus influenza; Plasmodium falciparum; Neisseria meningitidis; Streptococcus pneumoniae; Neisseria gonorrhoeae; salmonella serotype typhi; shigella; Vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; smallpox; allergies and cancers.

Examples of protozoal and other parasitic antigens include, but are not limited to, e.g., Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

In some embodiments, the viral antigen is from Herpes simplex virus (HSV), respiratory syncytial virus (RSV), metapneumovirus (hMPV), rhinovirus, parainfluenza (PIV), Epstein-Barr virus (EBV), Cytomegalovirus (CMV), JC virus (John Cunningham virus), BK virus, HIV, Zika virus, human coronavirus, norovirus, encephalitis virus, or Ebola. In some embodiments, the virus is an Orthomyxoviridae virus selected from the group consisting of Influenza A virus, Influenza B virus, Influenza C virus, and any subtype or reassortant thereof. In some embodiments, the virus is an Influenza A virus or any subtype or reassortant thereof, such as Influenza A virus subtype H1N1 (H1N1) or Influenza A virus subtype H5N1 (H5N1). In some embodiments, the virus is a Coronaviridae virus selected from the group consisting of alpha coronaviruses 229E (HCoV-229E), New Haven coronavirus NL63 (HCoV-NL63), beta coronaviruses OC43 (HCoV-OC43), coronavirus HKU1 (HCoV-HKU1), Severe Acute Respiratory Syndrome coronavirus (SARS-CoV), Middle East Respiratory Syndrome coronavirus (MERS-CoV), and Severe Acute Respiratory Syndrome Coronavirus 2 (SARS-CoV-2). In some embodiments, the virus is SARS-CoV, MERS-CoV, or SARS-CoV-2. In some embodiments, the virus is a Filoviridae virus selected from Ebola virus (EBOV) and Marburg virus (MARV). In some embodiments, the virus is a Flaviviridae virus selected from the group consisting of Zika virus (ZIKV), West Nile virus (WNV), Dengue virus (DENV), and Yellow Fever virus (YFV).

Antigens Involved in Autoimmune Diseases, Allergy, and Graft Rejection

In some embodiments, the target antigen or epitope is an antigen or epitope involved in autoimmune diseases, allergy, and/or graft rejection. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, inflammatory bowel disease (IBD), Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatibility antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease. In some embodiments, the target antigen is CD3, CD4, CD123, or CD8.

Immune Checkpoint Molecule

In some embodiments, the target antigen or epitope is an immune checkpoint molecule. Immune checkpoints are regulators of the immune system.

In some embodiments, the immune checkpoint molecule is a stimulatory immune checkpoint molecule. In some embodiments, the stimulatory immune checkpoint molecule is selected from the group consisting of CD27, CD28, OX40, ICOS, GITR, 4-1BB, CD27, CD40, CD3, and HVEM. Thus, in some embodiments, the antigen-binding protein (e.g., antibody, antigen-binding domain, or ligand/receptor-Fc fusion protein) described herein is an activator of a stimulatory immune checkpoint molecule, which can stimulate, activate, or increase the intensity of an immune response mediated by a stimulatory immune checkpoint molecule. The antibody or antigen-binding fragment described herein can be derived from any antibody known in the art that activates a stimulatory immune checkpoint molecule. In some embodiments, the antigen-binding fragment is a ligand or receptor of a stimulatory immune checkpoint molecule, e.g., can activate stimulatory immune checkpoint signaling. In some embodiments, the antigen-binding protein (e.g., antibody, antigen-binding domain, or ligand/receptor-Fc fusion protein) described herein is an antagonist of a stimulatory immune checkpoint molecule, which can reduce or block the intensity of an immune response mediated by a stimulatory immune checkpoint molecule.

In some embodiments, the immune checkpoint molecule is an inhibitory immune checkpoint molecule. In some embodiments, the inhibitory immune checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, and VISTA. In some embodiments, the inhibitory immune checkpoint molecule is PD-1, PD-L2, or PD-L1. In some embodiments, the inhibitory immune checkpoint molecule is CTLA-4. In some embodiments, the inhibitory immune checkpoint molecule is TIGIT. Thus, in some embodiments, the antigen-binding protein (e.g., antibody, antigen-binding domain, or ligand/receptor-Fc fusion protein) described herein is an immune checkpoint inhibitor, which totally or partially reduces, inhibits, or interferes with one or more inhibitory immune checkpoint molecules. The antibody or antigen-binding domain described herein can be derived from any antibody known in the art that serves as an immune checkpoint inhibitor. In some embodiments, the antigen-binding fragment is a ligand (e.g., CD155, PD-L2 or PD-L1) or receptor of an inhibitory immune checkpoint molecule (e.g., TIGIT or PD-1), e.g., can activate or stimulate an inhibitory immune checkpoint signaling (e.g., TIGIT or PD-1 signaling). In some embodiments, the antigen-binding protein (e.g., antibody, antigen-binding domain, or ligand/receptor-Fc fusion protein) described herein is an agonist of an inhibitory immune checkpoint molecule, which can stimulate, activate, or increase the intensity of an immune response mediated by an inhibitory immune checkpoint molecule.

PD-1 (programmed cell death protein 1) is a part of the B7/CD28 family of co-stimulatory molecules that regulate T-cell activation and tolerance, and thus antagonistic anti-PD-1 antibodies or PD-1 ligand-Fc fusion protein can be useful for overcoming tolerance. PD-1 has been defined as a receptor for B7-4. B7-4 can inhibit immune cell activation upon binding to an inhibitory receptor on an immune cell. Engagement of the PD-I/PD-L1 pathway results in inhibition of T-cell effector function, cytokine secretion and proliferation. (Turnis et al., OncoImmunology 1(7):1172-1174, 2012). High levels of PD-1 are associated with exhausted or chronically stimulated T cells. Moreover, increased PD-I expression correlates with reduced survival in cancer patients. Agents for down modulating PD-1, B7-4, and the interaction between B7-4 and PD-I inhibitory signal in an immune cell can result in enhancement of the immune response. Exemplary anti-PD-1 antibodies include, but are not limited to, pembrolizumab (e.g., Keytruda®), cemiplimab (Libtayo®), and nivolumab (e.g., Opdivo®). In some embodiments, the antibody or antigen-binding domain specifically recognizing PD-1 comprises heavy chain CDRs, light chain CDRs, or all six CDRs of pembrolizumab, cemiplimab, or nivolumab. In some embodiments, the antibody or antigen-binding domain specifically recognizing PD-1 comprises VH and/or VL of pembrolizumab, cemiplimab, or nivolumab. In some embodiments, the immunocytokine comprises a parental anti-PD-1 antibody (e.g., full-length antibody). In some embodiments, anti-PD-1 antibody or antigen binding fragment thereof comprises VH-CDR1, VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103. In some embodiments, the anti-PD-1 antibody or antigen binding fragment thereof comprises a VH comprising the sequence of SEQ ID NO: 102 and a VL comprising the sequence of SEQ ID NO: 103. In some embodiments, the anti-PD-1 antibody (e.g., parental full-length antibody) comprises two heavy chains each comprising the sequence of SEQ ID NO: 104 or 105, and two light chains each comprising the sequence of SEQ ID NO: 106. In some embodiments, the anti-PD-1 antibody (e.g., parental antibody) is a homodimeric full-length antibody. In some embodiments, the anti-PD-1 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

PD-L1 (programmed cell death-ligand 1) is also known as cluster of differentiation 274 (CD274) or B7 homolog 1 (B7-H1). PD-L1 serves as a ligand for PD-1 to play a major role in suppressing the immune system during particular events such as pregnancy, tissue allographs, autoimmune disease and other disease states such as hepatitis and cancer. The formation of PD-1 receptor/PD-L1 ligand complex transmits an inhibitory signal, which reduces the proliferation of CD8⁺ T cells at the lymph nodes. Exemplary anti-PD-L1 antibodies include, but are not limited to, atezolizumab (e.g., Tecentriq®), avelumab (e.g., Bavencio®), and durvalumab (e.g., IMFINZI™) In some embodiments, the antibody or antigen-binding domain specifically recognizing PD-L1 comprises heavy chain CDRs, light chain CDRs, or all six CDRs of atezolizumab, avelumab, or durvalumab. In some embodiments, the antibody or antigen-binding domain specifically recognizing PD-L1 comprises VH and/or VL of atezolizumab, avelumab, or durvalumab. In some embodiments, the immunocytokine comprises a parental anti-PD-L1 antibody (e.g., full-length antibody). In some embodiments, the anti-PD-L1 antibody or antigen binding fragment thereof comprises: i) a VH-CDR1 comprising the sequence of SEQ ID NO: 243; ii) a VH-CDR2 comprising the sequence of SEQ ID NO: 244; iii) a VH-CDR3 comprising the sequence of SEQ ID NO: 245; iv) a VL-CDR1 comprising the sequence of SEQ ID NO: 246; v) a VL-CDR2 comprising the sequence of SEQ ID NO: 247; and vi) a VL-CDR3 comprising the sequence of SEQ ID NO: 248. In some embodiments, the anti-PD-L1 antibody or antigen binding fragment thereof comprises a VH comprising the sequence of SEQ ID NO: 137 and a VL comprising the sequence of SEQ ID NO: 138. In some embodiments, the anti-PD-L1 antibody (e.g., parental full-length antibody) comprises two heavy chains each comprising the sequence of SEQ ID NO: 139, and two light chains each comprising the sequence of SEQ ID NO: 140. In some embodiments, the anti-PD-L1 antibody (e.g., parental full-length antibody) comprises two heavy chains each comprising the sequence of SEQ ID NO: 141, and two light chains each comprising the sequence of SEQ ID NO: 142. In some embodiments, the anti-PD-L1 antibody (e.g., parental antibody) is a homodimeric full-length antibody. In some embodiments, the anti-PD-L1 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62. In some embodiments, the parental antigen-binding protein comprises (e.g., consists essentially of, or consists of) two antigen-binding polypeptides each comprising (e.g., consists essentially of, or consists of) from N′ to C′: PD-L1 extracellular domain or portion thereof—optional peptide linker—hinge region—Fc domain subunit or portion thereof. In some embodiments, PD-L1 comprises the sequence of SEQ ID NO: 249. In some embodiments, the PD-L1 extracellular domain comprises the sequence of SEQ ID NO: 250. In some embodiments, the PD-L1 extracellular domain or portion thereof is derived from wildtype (e.g., wildtype human) PD-L1. In some embodiments, the PD-L1 extracellular domain or portion thereof comprises one or more mutations (e.g., deletion, insertion, or replacement). In some embodiments, the mutated PD-L1 extracellular domain or portion thereof has increased affinity to PD-1 compared to wildtype PD-L1 extracellular domain or portion thereof. In some embodiments, the mutated PD-L1 extracellular domain or portion thereof has reduced affinity to PD-1 compared to wildtype PD-L1 extracellular domain or portion thereof. In some embodiments, the PD-Li-hinge-Fc fusion protein (e.g., parental fusion protein) is a homodimeric PD-Li-hinge-Fc fusion protein, i.e., two PD-L1-hinge-Fc polypeptides are identical. In some embodiments, the PD-L1-hinge-Fc fusion protein (e.g., parental fusion protein) is a heterodimeric full-length antibody, e.g., one PD-L1-hinge-Fc polypeptide comprises an Fc domain subunit comprising the sequence of SEQ ID NO: 61 and the other PD-L1-hinge-Fc polypeptide comprises an Fc domain subunit comprising the sequence of SEQ ID NO: 62. In some embodiments, such as within the parental PD-Li-hinge-Fc fusion protein, PD-L1 (e.g., PD-L1 extracellular domain) and the hinge region are connected by a peptide linker, such as any peptide linkers described herein. In some embodiments, the peptide linker comprises the sequence of SEQ ID NO: 203, 206, or 213.

PD-L2 (programmed cell death 1 ligand 2, B7-DC, CD273) is another immune checkpoint receptor ligand of PD-1. PD-L2 plays a role in negative regulation of the adaptive immune response. Engagement of PD-1 by PD-L2 dramatically inhibits T cell receptor (TCR)-mediated proliferation and cytokine production by T cells. At low antigen concentrations, PD-L2-PD-1 interactions inhibit strong B7-CD28 signals. In contrast, at high antigen concentrations, PD-L2-PD-1 interactions reduce cytokine production but do not inhibit T cell proliferation. In some embodiments, the antigen-binding protein (e.g., parental antigen-binding protein) is a PD-L2 (e.g., full length or portion thereof)-hinge-Fc fusion protein. In some embodiments, the antigen-binding protein comprises (e.g., consists essentially of, or consists of) two antigen-binding polypeptides each comprising (e.g., consists essentially of, or consists of) from N′ to C′: PD-L2 extracellular domain or portion thereof—optional peptide linker—hinge region—Fc domain subunit or portion thereof. In some embodiments, PD-L2 comprises the sequence of SEQ ID NO: 175. In some embodiments, the PD-L2 extracellular domain comprises the sequence of SEQ ID NO: 176. In some embodiments, the PD-L2 extracellular domain or portion thereof is derived from wildtype (e.g., wildtype human) PD-L2. In some embodiments, the PD-L2 extracellular domain or portion thereof comprises one or more mutations (e.g., deletion, insertion, or replacement). In some embodiments, the mutated PD-L2 extracellular domain or portion thereof has increased affinity to PD-1 compared to wildtype PD-L2 extracellular domain or portion thereof. In some embodiments, the mutated PD-L2 extracellular domain or portion thereof has reduced affinity to PD-1 compared to wildtype PD-L2 extracellular domain or portion thereof. In some embodiments, the PD-L2-hinge-Fc fusion protein (e.g., parental fusion protein) comprises two PD-L2-hinge-Fc polypeptides each comprising the sequence of SEQ ID NO: 177. In some embodiments, the PD-L2-hinge-Fc fusion protein (e.g., parental fusion protein) is a homodimeric PD-L2-hinge-Fc fusion protein, i.e., two PD-L2-hinge-Fc polypeptides are identical. In some embodiments, the PD-L2-hinge-Fc fusion protein (e.g., parental fusion protein) is a heterodimeric full-length antibody, e.g., one PD-L2-hinge-Fc polypeptide comprises an Fc domain subunit comprising the sequence of SEQ ID NO: 61 and the other PD-L2-hinge-Fc polypeptide comprises an Fc domain subunit comprising the sequence of SEQ ID NO: 62. In some embodiments, such as within the parental PD-L2-hinge-Fc fusion protein, PD-L2 (e.g., PD-L2 extracellular domain) and the hinge region are connected by a peptide linker, such as any peptide linkers described herein. In some embodiments, the peptide linker comprises the sequence of SEQ ID NO: 203, 206, or 213.

Cytotoxic T-Lymphocyte-Associated protein 4 (CTLA-4, or CD152) is a homolog of CD28, and is known as an inhibitory immune checkpoint molecule upregulated on activated T-cells. CTLA-4 also binds to B7-1 and B7-2, but with greater affinity than CD28. The interaction between B7 and CTLA-4 dampens T cell activation, which constitutes an important mechanism of tumor immune escape. Anti-CTLA-4 antibody therapy has shown promise in a number of cancers, such as melanoma. Exemplary anti-CTLA-4 antibodies include, but are not limited to, ipilimumab (e.g., Yervoy®). In some embodiments, the antibody or antigen-binding domain specifically recognizing CTLA-4 comprises heavy chain CDRs, light chain CDRs, or all six CDRs of ipilimumab. In some embodiments, the antibody or antigen-binding domain specifically recognizing CTLA-4 comprises VH and/or VL of ipilimumab. In some embodiments, the immunocytokine comprises a parental anti-CTLA-4 antibody (e.g., full-length antibody). In some embodiments, the anti-CTLA-4 antibody or antigen binding fragment thereof comprises VH-CDR1, VH-CDR2, and VH-CDR3 of a VH comprising the sequence of SEQ ID NO: 125, and VL-CDR1, VL-CDR2, and VL-CDR3 of a VL comprising the sequence of SEQ ID NO: 126. In some embodiments, the anti-CTLA-4 antibody or antigen binding fragment thereof comprises a VH comprising the sequence of SEQ ID NO: 125 and a VL comprising the sequence of SEQ ID NO: 126. In some embodiments, the anti-CTLA-4 antibody (e.g., parental full-length antibody) comprises two heavy chains each comprising the sequence of SEQ ID NO: 127 or 128, and two light chains each comprising the sequence of SEQ ID NO: 129. In some embodiments, the anti-CTLA-4 antibody or antigen binding fragment thereof comprises a VH of a heavy chain comprising the sequence of SEQ ID NO: 128 and a VL of a light chain comprising the sequence of SEQ ID NO: 129. In some embodiments, the anti-CTLA-4 antibody (e.g., parental antibody) is a homodimeric full-length antibody. In some embodiments, the anti-CTLA-4 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

TIGIT (also called T cell immunoreceptor with Ig and ITIM domains or VSTM3) is an immune receptor on some T cells and NK cells. For example, TIGIT is found to overexpress on tumor antigen-specific CD8+ T cells and CD8+ tumor infiltrating lymphocytes (TILs) in melanoma. When TIGIT is bound by its ligand CD155 (PVR), which is expressed on tumor cells, dendritic cells, macrophages, etc., TIGIT sends an inhibitory signal to T cells or NK cells.

Cell Surface Ligand or Receptor

In some embodiments, the target antigen or epitope is a ligand or receptor or portion thereof, such as extracellular domain of a ligand/receptor. In some embodiments, the ligand or receptor is derived from a molecule selected from the group consisting of IL-2, IL-2Rα (CD25), IL-3Ru (CD123), PD-1, PD-L1, PD-L2, CD155, NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL and/or BAFF, which can bind to BCMA. In some embodiments, the receptor is an FcR and the ligand is an Fc-containing molecule. In some embodiments, the FcR is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b). In some embodiments, the target antigen or epitope is PD-1, such as extracellular domain (or portion thereof) of PD-1. In some embodiments, the antigen-binding fragment of the antigen-binding polypeptide is PD-L2 or PD-L1, such as extracellular domain (or portion thereof) of PD-L2 or PD-L1. In some embodiments, the PD-L2 extracellular domain comprises the sequence of SEQ ID NO: 176. In some embodiments, the PD-L1 extracellular domain comprises the sequence of SEQ ID NO: 250. In some embodiments, the antigen-binding fragment of the antigen-binding polypeptide is CD155, such as extracellular domain (or portion thereof) of CD155. In some embodiments, the CD155 extracellular domain comprises the sequence of SEQ ID NO: 267.

The receptor of IL-2, interleukin-2 receptor (IL-2R), is a heterotrimeric protein expressed on the surface of certain immune cells, such as lymphocytes. IL-2R has three forms generated by different combinations of α chain (IL-2Rα, CD25, Tac antigen), β chain (IL-2Rβ, CD122), and γ chain (IL-2Rγ, γ_{c}, common gamma chain, or CD132). IL-2Rα binds IL-2 with low affinity, and the complex of IL-2Rβ and IL-2Ry binds IL-2 with intermediate affinity, primarily on memory T cells and NK cells. The complex of all α, β, and γ chains bind IL-2 with high affinity on activated T cells and regulatory T cells (Tregs). CD25 (IL-2Rα) plays a critical role in the development and maintenance of Tregs, and may play a role in Treg expression of CD62L, which is required for the entry of Tregs into lymph nodes (Malek and Bayer, 2004). CD25 is a marker for activated T cells and Treg. Experimental data suggested an immunosuppressive capacity of anti-CD25 that significantly delayed rejection of heart allografts in the mouse (Kirkman et al., 1985) and of renal allografts in nonhuman primates (Reed et al., 1989). Exemplary anti-CD25 antibodies include, but are not limited to basiliximab (e.g., Simulect®), daclizumab (e.g., Zinbryta®). In some embodiments, the antibody or antigen-binding domain specifically recognizing CD25 comprises heavy chain CDRs, light chain CDRs, or all six CDRs of basiliximab or daclizumab. In some embodiments, the antibody or antigen-binding domain specifically recognizing CD25 comprises VH and/or VL of basiliximab or daclizumab. In some embodiments, the immunocytokine comprises a parental anti-CD25 antibody (e.g., full-length antibody). In some embodiments, the anti-CD25 antibody or antigen binding fragment thereof comprises VH-CDR1, VH-CDR2, and VH-CDR3 of a VH comprising the sequence of SEQ ID NO: 162, and VL-CDR1, VL-CDR2, and VL-CDR3 of a VL comprising the sequence of SEQ ID NO: 163. In some embodiments, the anti-CD25 antibody or antigen binding fragment thereof comprises a VH comprising the sequence of SEQ ID NO: 162 and a VL comprising the sequence of SEQ ID NO: 163. In some embodiments, the anti-CD25 antibody (e.g., parental full-length antibody) comprises two heavy chains each comprising the sequence of SEQ ID NO: 164, and two light chains each comprising the sequence of SEQ ID NO: 165. In some embodiments, the anti-CD25 antibody or antigen binding fragment thereof comprises VH-CDR1, VH-CDR2, and VH-CDR3 of a heavy chain comprising the sequence of SEQ ID NO: 166, and VL-CDR1, VL-CDR2, and VL-CDR3 of a light chain comprising the sequence of SEQ ID NO: 167. In some embodiments, the anti-CD25 antibody or antigen binding fragment thereof comprises a VH of a heavy chain comprising the sequence of SEQ ID NO: 166, and a VL of a light chain comprising the sequence of SEQ ID NO: 167. In some embodiments, the anti-CD25 antibody (e.g., parental full-length antibody) comprises two heavy chains each comprising the sequence of SEQ ID NO: 166, and two light chains each comprising the sequence of SEQ ID NO: 167. In some embodiments, the anti-CD25 antibody (e.g., parental antibody) is a homodimeric full-length antibody. In some embodiments, the anti-CD25 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

Immune Cell Surface Antigen

In some embodiments, the target antigen or epitope is an immune cell surface antigen or epitope. Immune cells have different cell surface molecules. For example CD3 is a cell surface molecule on T-cells, whereas CD16, NKG2D, or NKp30 are cell surface molecules on NK cells, and CD3 or an invariant T-cell receptor (TCR) are the cell surface molecules on NKT-cells. In some embodiments, wherein the immune cell is a T-cell, the activation molecule is one or more of CD3, e.g., CD3R, CD36, or CD3γ; or CD2, CD4, CD8, CD27, CD28, CD40, CD134, CD137, CD278, inhibitory immune checkpoint molecules (e.g., CTLA-4, PD-1, TIM3, BTLA, VISTA, LARG-3, or TIGIT), and stimulatory immune checkpoint molecules (CD27, CD28, CD137, OX40, GITR, or HVEM). In some embodiments, wherein the immune cell is a B cell, the cell surface molecule is CD19, CD20, or CD138. In other some embodiments, wherein the immune cell is a NK cell, the cell surface molecule is CD16, CD56 (NCAM), NKp46, NKp44, CD244, CD226, TIGIT, CD96, LAG3, TIM3, PD-1, KLRG1, CD161, CD94/NKG2, KIR, NKG2D, or NKp30. In some embodiments, wherein the immune cell is a NKT-cell, the cell surface molecule is CD3 or an invariant TCR. In some embodiments, wherein the immune cell is a myeloid dendritic cell (mDC), the cell surface molecule is CD11c, CD11b, CD13, CD45RO, or CD33. In some embodiments, wherein the immune cell is a plasma dendritic cell (pDC), the cell surface molecule is CD123, CD62L, CD45RA, or CD36. In some embodiments, wherein the immune cell is a macrophage, the cell surface molecule is CD163 or CD206. In some embodiments, the immune cell is selected from the group consisting of a monocyte, a dendritic cell, a macrophage, a B cell, a killer T cell (T_{c}, cytotoxic T lymphocyte, or CTL), a helper T cell (Th), a regulatory T cells (Treg), a γ6 T cell, a natural killer T (NKT) cell, and a natural killer (NK) cell.

In some embodiments, the immune cell surface antigen is selected from the group consisting of CD3 (e.g., CD3R, CD36, CD3γ), CD4, CD5, CD8, CD16, CD27, CD28, CD40, CD64, CD89, CD134, CD137, CD278, NKp46, NKp30, NKG2D, TCRa, TCRP, TCRy, and TCR6. In some embodiments, the immune cell surface antigen is CD3, CD4, or CD8.

CD3 comprises three different polypeptide chains (ε, δ and γ chains), is an antigen expressed by T cells, including cytotoxic T cell (CD8+ naive T cells) and T helper cells (CD4+ naive T cells). The three CD3 polypeptide chains associate with the TCR and the ξ-chain to form the TCR complex, which has the function of activating signaling cascades in T cells. Currently, many therapeutic strategies target the TCR signal transduction to treat diseases using anti-human CD3 monoclonal antibodies. The CD3 specific antibody OKT3 is the first monoclonal antibody approved for human therapeutic use, and is clinically used as an immunomodulator for the treatment of allogenic transplant rejections. Otelixizumab (TRX4) is a monoclonal antibody specifically targeting CD3R, and is being developed for the treatment of type 1 diabetes and other autoimmune diseases. In some embodiments, the antibody or antigen-binding domain specifically recognizing CD3 comprises heavy chain CDRs, light chain CDRs, or all six CDRs of OKT3 or otelixizumab. In some embodiments, the antibody or antigen-binding domain specifically recognizing CD3 comprises VH and/or VL of OKT3 or otelixizumab. In some embodiments, the anti-CD3 antibody or antigen binding fragment thereof does not compete with OKT3 antibody for binding to CD3, and is unable to activate TCR signaling (i.e., non-active anti-CD3 antibody). In some embodiments, the anti-CD3 antibody or antigen binding fragment specifically recognizes CD3. In some embodiments, the immunocytokine comprises a parental anti-CD3 antibody (e.g., full-length antibody). In some embodiments, anti-CD3 (e.g., CD3E) antibody or antigen binding fragment thereof comprises: i) a VH-CDR1 comprising the sequence of SEQ ID NO: 85; ii) a VH-CDR2 comprising the sequence of SEQ ID NO: 86; iii) a VH-CDR3 comprising the sequence of SEQ ID NO: 87; iv) a VL-CDR1 comprising the sequence of SEQ ID NO: 88; v) a VL-CDR2 comprising the sequence of SEQ ID NO: 89; and vi) a VL-CDR3 comprising the sequence of SEQ ID NO: 90. In some embodiments, the anti-CD3 (e.g., CD3E) antibody or antigen binding fragment thereof comprises a VH comprising the sequence of SEQ ID NO: 91 and a VL comprising the sequence of SEQ ID NO: 92. In some embodiments, the anti-CD3 antibody (e.g., parental full-length antibody) comprises two heavy chains each comprising the sequence of SEQ ID NO: 93, and two light chains each comprising the sequence of SEQ ID NO: 94. In some embodiments, the anti-CD3 antibody (e.g., parental antibody) is a homodimeric full-length antibody. In some embodiments, the anti-CD3 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

CD4 is a glycoprotein expressed on the surface of immune cells such as T helper cells (CD4+T helper cells), monocytes, macrophages, and dendritic cells. CD4 is a co-receptor of the TCR and assists TCR in communicating with antigen-presenting cells. Exemplary anti-CD4 antibodies include, but are not limited to, Ibalizumab (e.g., Trogarzo®), MAX.16H5, and IT1208. MAX.16H5 is an anti-human CD4 antibody applied intravenously in clinical trials for the treatment of autoimmune diseases (e.g., rheumatoid arthritis) and acute late-onset rejection after transplantation of a renal allograft. IT1208 is a defucosylated humanized anti-CD4 depleting antibody currently under clinical trial for treating advanced solid tumors. In some embodiments, Ibalizumab was used as the parental anti-CD4 antibody (e.g., full-length antibody). Ibalizumab is a non-immunosuppressive humanized monoclonal antibody that has been approved by FDA in HV treatment. In some embodiments, the antigen-binding domain specifically recognizing CD4 comprises heavy chain CDRs, light chain CDRs, or all six CDRs of Ibalizumab, MAX.16H5 or IT1208. In some embodiments, the antigen-binding domain specifically recognizing CD4 comprises VH and/or VL of Ibalizumab, MAX.16H5 or IT1208. In some embodiments, the immunocytokine comprises a parental anti-CD4 antibody (e.g., full-length antibody). In some embodiments, anti-CD4 antibody or antigen binding fragment thereof comprises: i) a VH-CDR1 comprising the sequence of SEQ ID NO: 67; ii) a VH-CDR2 comprising the sequence of SEQ ID NO: 68; iii) a VH-CDR3 comprising the sequence of SEQ ID NO: 69; iv) a VL-CDR1 comprising the sequence of SEQ ID NO: 70; v) a VL-CDR2 comprising the sequence of SEQ ID NO: 71; and vi) a VL-CDR3 comprising the sequence of SEQ ID NO: 72. In some embodiments, the anti-CD4 antibody or antigen binding fragment thereof comprises a VH comprising the sequence of SEQ ID NO: 73 and a VL comprising the sequence of SEQ ID NO: 74. In some embodiments, the anti-CD4 antibody (e.g., parental full-length antibody) comprises two heavy chains each comprising the sequence of SEQ ID NO: 75 or 76, and two light chains each comprising the sequence of SEQ ID NO: 77. In some embodiments, the anti-CD4 antibody (e.g., parental antibody) is a homodimeric full-length antibody. In some embodiments, the anti-CD4 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

CD8 is a transmembrane glycoprotein that serves as a co-receptor for TCR. CD8 binds to and is specific for MHC class I protein. The most common form of CD8 is composed of a CD8-α and CD8-β chain. CD8 is predominantly expressed on the surface of cytotoxic T cells, but can also be found on natural killer cells, cortical thymocytes, and dendritic cells. CD8 is a marker for cytotoxic T cell population. CD8 is expressed in T cell lymphoblastic lymphoma and hypo-pigmented mycosis fungoides. Exemplary anti-CD8 antibodies include, but are not limited to, G10⁻¹, OKT8, YTC182.20, 4B11, and DK25. In some embodiments, the antigen-binding domain specifically recognizing CD8 comprises heavy chain CDRs, light chain CDRs, or all six CDRs of any of G10⁻¹, OKT8, YTC182.20, 4B11, or DK25. In some embodiments, the antigen-binding domain specifically recognizing CD8 comprises VH and/or VL of any of G10⁻¹, OKT8, YTC182.20, 4B11, or DK25. In some embodiments, the immunocytokine comprises a parental anti-CD8 antibody (e.g., full-length antibody). In some embodiments, anti-CD8 antibody or antigen binding fragment thereof comprises VH-CDR1, VH-CDR2, and VH-CDR3 of a VH comprising the sequence of SEQ ID NO: 114, and VL-CDR1, VL-CDR2, and VL-CDR3 of a VL comprising the sequence of SEQ ID NO: 115. In some embodiments, the anti-CD8 antibody or antigen binding fragment thereof comprises a VH comprising the sequence of SEQ ID NO: 114 and a VL comprising the sequence of SEQ ID NO: 115. In some embodiments, the anti-CD8 antibody (e.g., parental full-length antibody) comprises two heavy chains each comprising the sequence of SEQ ID NO: 116, and two light chains each comprising the sequence of SEQ ID NO: 117. In some embodiments, the anti-CD8 antibody (e.g., parental antibody) is a homodimeric full-length antibody. In some embodiments, the anti-CD8 antibody (e.g., parental antibody) is a heterodimeric full-length antibody, e.g., KIH antibody, for example, one heavy chain comprises an Fc domain subunit comprising the sequence of SEQ ID NO: 61 and the other heavy chain comprises an Fc domain subunit comprising the sequence of SEQ ID NO: 62.

### Hinge

Hinge connects the Fd region (VH and CH1 domains) and the Fc region of a heavy chain of an immunoglobulin. In some embodiments, a hinge region connects an antigen-binding fragment (e.g., ligand, receptor, VHH, scFv, or Fab) and an Fc domain subunit or portion thereof (e.g., CH2+CH3, or CH2 only). The hinge region, found in IgG, IgA, and IgD immunoglobulin classes, acts as a flexible spacer that allows the Fab portion of an immunoglobulin to move freely in space relative to the Fc region. The hinge domains are structurally diverse, varying in both sequence and length among immunoglobulin classes and subclasses. The heavy chains are inter-connected via disulfide bonds in the hinge region. According to crystallographic studies, the immunoglobulin hinge region can be further subdivided structurally and functionally into three regions: the upper hinge, the core, and the lower hinge. See Shin et al., Immunological Reviews 130:87 (1992). The upper hinge includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges. The lower hinge region joins the amino terminal end of, and includes residues in, the CH2 domain. Id. The hinge region of a human IgG1 antibody corresponds to amino acids 216-230 according to the EU numbering as set forth in Kabat. The core hinge region of human IgG1 contains the sequence Cys-Pro-Pro-Cys that, when dimerized by disulfide bond formation, results in a cyclic octapeptide believed to act as a pivot, thus conferring flexibility. Conformational changes permitted by the structure and flexibility of the immunoglobulin hinge region polypeptide sequence may affect the effector functions of the Fc portion of the antibody.

In some embodiments, the hinge region may contain one or more glycosylation site(s), which include a number of structurally distinct types of sites for carbohydrate attachment. For example, IgA₁ contains five glycosylation sites within a 17 amino acid segment of the hinge region, conferring exceptional resistance of the hinge region polypeptide to intestinal proteases, considered an advantageous property for a secretory immunoglobulin.

In some embodiments, the immunocytokine comprises a hinge region that is present in a naturally occurring parental antibody. For example, the parental antibody is an IgG1 antibody, and the hinge region of the antibody or antigen-binding fragment within the immunocytokine described herein is an IgG1-type hinge region. In some embodiments, the immunocytokine contains a modification of the antibody heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class (e.g., IgG, IgA, or IgE) and subclass (e.g., IgG1, IgG2, IgG3, and IgG4, etc.). For example, an IgG1, IgG2, or IgG3 antibody may contain an IgG4-type hinge region. In some embodiments, the hinge region or a portion thereof comprises a mutation, e.g., deletion, insertion, or replacement, at one or more of the upper hinge, the core, and the lower hinge of the hinge region, as long as inter-chain disulfide bond(s) can still be formed, the immunocytokine has flexibility to ensure target antigen-antigen binding fragment binding, masking cytokine activity in the absence of target antigen-antibody binding, while unmasking cytokine activity in the presence of target antigen-antibody binding, providing flexibility and/or sufficient space between two cytokine subunits or two cytokine moieties to ensure proper cytokine activity (binding affinity and/or bioactivity), and/or optionally does not abolish effector function(s) of the Fc portion. In some embodiments, the hinge region is or is derived from a human IgG1, IgG2, IgG3, or IgG4 hinge. In some embodiments, the hinge region is a mutated human IgG1, IgG2, IgG3, or IgG4 hinge. In some embodiments, one or more mutations, e.g., deletion, insertion, or replacement, are introduced at one or more of the upper hinge, the core, and the lower hinge of the hinge region in order to reduce or eliminate effector function (e.g., ADCC, and/or CDC) of the Fc domain, such as L234 and/or L235 mutations in the IgG1 lower hinge region, e.g., one or two of L234A, L234K, L234D, L235E, L235K, and L235A mutations. In some embodiments, the hinge region comprises L234K and L235K mutations. In some embodiments, the hinge region comprises L234D and L235E mutations. In some embodiments, the hinge region is truncated or mutated with less cysteines in order to reduce disulfide bond mis-pairing during dimerization of the Fc domain. In some embodiments, one or more asymmetric charged mutation(s) is introduced into the lower hinge to facilitate heterodimer formation, e.g., one polypeptide comprises L234K+L235K in the IgG1 lower hinge region, while the pairing polypeptide comprises L234D+L235E in the IgG1 lower hinge region. In some embodiments, the hinge region comprises the amino acid sequence of EPKSCDKTHTCPPCPAPELLGGP (SEQ ID NO: 40). In some embodiments, the hinge region is an IgG1 hinge comprising L234K and L235K mutations. In some embodiments, the hinge region comprises the amino acid sequence of EPKSCDKTHTCPPCPAPEKKGGP (SEQ ID NO: 41). In some embodiments, the hinge region is an IgG1 hinge comprising L234D and L235E mutations. In some embodiments, the hinge region comprises the amino acid sequence of EPKSCDKTHTCPPCPAPEDEGGP (SEQ ID NO: 42). In some embodiments, the hinge region comprises the amino acid sequence of ERKCCVECPPCPAPPVAGP (SEQ ID NO: 46). In some embodiments, the hinge region comprises the amino acid sequence of ESKYGPPCPSCPAPEFLGGP (SEQ ID NO: 47). In some embodiments, the hinge region comprises the amino acid sequence of ESKYGPPCPPCPAPEFLGGP (SEQ ID NO: 58). In some embodiments, the hinge region comprises the amino acid sequence of any of EPKSCDKDKTHTCPPCPAPELLGGP (SEQ ID NO: 43), EPKSCDKDKTHTCPPCPAPEKKGGP (SEQ ID NO: 44), or EPKSCDKDKTHTCPPCPAPEDEGGP (SEQ ID NO: 45). In some embodiments, the hinge region comprises the amino acid sequence of any of EPKSCDKPDKTHTCPPCPAPELLGGP (SEQ ID NO: 55), EPKSCDKPDKTHTCPPCPAPEKKGGP (SEQ ID NO: 56), EPKSCDKPDKTHTCPPCPAPEDEGGP (SEQ ID NO: 57), or EPPKSCDKTHTCPPCPAPELLGGP (SEQ ID NO: 59). In some embodiments, the hinge region, such as the hinge N′ portion, comprises the amino acid sequence of any of EPKSCDKP (SEQ ID NO: 54), EPKSCDK (SEQ ID NO: 48), or EPKSC (SEQ ID NO: 49). In some embodiments, the hinge region comprises the amino acid sequence of DKTHT (SEQ ID NO: 53). In some embodiments, the hinge region, such as the hinge C′ portion, comprises the amino acid sequence of any of DKTHTCPPCPAPELLGGP (SEQ ID NO: 50), DKTHTCPPCPAPEKKGGP (SEQ ID NO: 51), or DKTHTCPPCPAPEDEGGP (SEQ ID NO: 52).

In some embodiments, the cytokine or variant thereof described herein is positioned at the N-terminus of the hinge region of a heavy chain of a full-length antibody, i.e., positioned between the C-terminus of the CH1 and the N-terminus of the hinge region of the heavy chain of the full-length antibody. In some embodiments, the heavy chain fusion polypeptide comprises from N′ to C′: VH-CH1-cytokine moiety-hinge-CH2-CH3. In some embodiments, the cytokine or variant thereof is positioned at the N-terminus of the hinge region between an antigen-binding fragment (e.g., ligand, receptor, VHH, scFv, or Fab) and an Fc domain subunit or portion thereof (e.g., CH2-CH3, or CH2). For example, in some embodiments, the immunocytokine comprises a polypeptide of any of from N′ to C′: (1) VH-cytokine moiety-hinge-CH2-CH3; (2) VL-cytokine moiety-hinge-CH2-CH3; (3) VH-optional linker-VL-cytokine moiety-hinge-CH2-CH3; (4) VL-optional linker-VH-cytokine moiety-hinge-CH2-CH3; (5) VH-CH1-cytokine moiety-hinge-CH2-CH3; (6) VH-cytokine moiety-hinge-CH2; (7) VL-cytokine moiety-hinge-CH2; (8) VH-optional linker-VL-cytokine moiety-hinge-CH2; (9) VL-optional linker-VH-cytokine moiety-hinge-CH2; (10) VH-CH1-cytokine moiety-hinge-CH2; (11) ligand-optional linker-cytokine moiety-hinge-CH2-CH3; (12) ligand-optional linker-cytokine moiety-hinge-CH2; (13) receptor-optional linker-cytokine moiety-hinge-CH2-CH3; or (14) receptor-optional linker-cytokine moiety-hinge-CH2. In some embodiments, the cytokine or variant thereof is positioned at the N-terminus of a hinge region comprising the sequence of any of SEQ ID NOs: 50-52.

In some embodiments, the cytokine or variant thereof described herein is positioned at the C-terminus of the hinge region of a heavy chain of a full-length antibody, i.e., the heavy chain fusion polypeptide comprises from N′ to C′: VH-CH1-hinge-cytokine moiety-CH2-CH3. In some embodiments, the cytokine or variant thereof is positioned at the C-terminus of the hinge region between an antigen-binding fragment (e.g., ligand, receptor, VHH, scFv, or Fab) and an Fc domain subunit or portion thereof (e.g., CH2). For example, in some embodiments, the immunocytokine comprises a polypeptide of any of from N′ to C′: (1) VH-hinge-cytokine moiety-CH2-CH3; (2) VL-hinge-cytokine moiety-CH2-CH3; (3) VH-optional linker-VL-hinge-cytokine moiety-CH2-CH3; (4) VL-optional linker-VH-hinge-cytokine moiety-CH2-CH3; (5) VH-CH1-hinge-cytokine moiety-CH2-CH3; (6) VH-hinge-cytokine moiety-CH2; (7) VL-hinge-cytokine moiety-CH2; (8) VH-optional linker-VL-hinge-cytokine moiety-CH2; (9) VL-optional linker-VH-hinge-cytokine moiety-CH2; (10) VH-CH1-hinge-cytokine moiety-CH2; (11) ligand-hinge-cytokine moiety-CH2-CH3; (12) ligand-hinge-cytokine moiety-CH2; (13) receptor-hinge-cytokine moiety-CH2-CH3; or (14) receptor-hinge-cytokine moiety-CH2. In some embodiments, the cytokine or variant thereof is positioned at the C-terminus of a hinge region comprising the sequence of any of SEQ ID NOs: 40-47, 50-52, and 55-59.

In some embodiments, the cytokine or variant thereof described herein is positioned within the hinge region of a heavy chain of a full-length antibody, i.e., the heavy chain fusion polypeptide comprises from N′ to C′: VH-CH1-hinge N′ portion-cytokine moiety-hinge C′ portion-CH2-CH3. In some embodiments, the cytokine or variant thereof replaces a portion of the hinge region. In some embodiments, the cytokine or variant thereof is inserted within the hinge region, without deleting any hinge amino acid. In some embodiments, the cytokine or variant thereof is inserted within a hinge region comprising the sequence of any of SEQ ID NOs: 40-47 and 55-59. In some embodiments, the cytokine or variant thereof is inserted between an N′ hinge portion comprising the sequence of any of SEQ ID NOs: 48, 49, and 54, and a C′ hinge portion comprising the sequence of any of SEQ ID NOs: 50-52. In some embodiments, the cytokine or variant thereof with a peptide linker fused to the N′ of the cytokine or variant thereof is inserted within the hinge region. In some embodiments, the cytokine or variant thereof with a peptide linker fused to the C′ of the cytokine or variant thereof is inserted within the hinge region. For example, in some embodiments, the hinge-cytokine portion comprises a structure of from N′ to C′: hinge N′ portion-optional N′ peptide linker-cytokine moiety-optional C′ peptide linker-hinge C′ portion.

In some embodiments, the hinge region is an IgG1 hinge, and the cytokine or variant thereof is inserted between “EPKSC” (SEQ ID NO: 49) and “DKTHT” (SEQ ID NO: 53). In some embodiments, the N′ peptide linker comprises the amino acid sequence of DKP (SEQ ID NO: 231) or P (SEQ ID NO: 242). Hence in some embodiments, the cytokine or variant thereof is inserted between an additionally introduced “DKP” and the “DKTHT” sequence. In some embodiments, the N′ peptide linker comprises the amino acid sequence of DKPGS (SEQ ID NO: 232), PGS (SEQ ID NO: 233), or GS (SEQ ID NO: 234). In some embodiments, the N′ peptide linker comprises the amino acid sequence of DKPGSG (SEQ ID NO: 235), PGSG (SEQ ID NO: 236), or GSG (SEQ ID NO: 203). In some embodiments, the N′ peptide linker comprises the amino acid sequence of DKPGSGS (SEQ ID NO: 237), PGSGS (SEQ ID NO: 238), or GSGS (SEQ ID NO: 239). In some embodiments, the N′ peptide linker comprises the amino acid sequence of DKPGSGGGGG (SEQ ID NO: 240), PGSGGGGG (SEQ ID NO: 241), GSGGGGG (SEQ ID NO: 206). In some embodiments, the cytokine or variant thereof is positioned within the hinge region between an antigen-binding fragment (e.g., ligand, receptor, VHH, scFv, or Fab) and an Fc domain subunit or portion thereof (e.g., CH2). For example, in some embodiments, the immunocytokine comprises a polypeptide of any of from N′ to C′: (1) VH-hinge N′ portion-optional N′ peptide linker-cytokine moiety-optional C′ peptide linker-hinge C′ portion-CH2-CH3; (2) VL-hinge N′ portion-optional N′ peptide linker-cytokine moiety-optional C′ peptide linker-hinge C′ portion-CH2-CH3; (3) VH-optional linker-VL-hinge N′ portion-optional N′ peptide linker-cytokine moiety-optional C′ peptide linker-hinge C′ portion-CH2-CH3; (4) VL-optional linker-VH-hinge N′ portion-optional N′ peptide linker-cytokine moiety-optional C′ peptide linker-hinge C′ portion-CH2-CH3; (5) VH-CH1-hinge N′ portion-optional N′ peptide linker-cytokine moiety-optional C′ peptide linker-hinge C′ portion-CH2-CH3; (6) VH-hinge N′ portion-optional N′ peptide linker-cytokine moiety-optional C′ peptide linker-hinge C′ portion-CH2; (7) VL-hinge N′ portion-optional N′ peptide linker-cytokine moiety-optional C′ peptide linker-hinge C′ portion-CH2; (8) VH-optional linker-VL-hinge N′ portion-optional N′ peptide linker-cytokine moiety-optional C′ peptide linker-hinge C′ portion-CH2; (9) VL-optional linker-VH-hinge N′ portion-optional N′ peptide linker-cytokine moiety-optional C′ peptide linker-hinge C′ portion-CH2; (10) VH-CH1-hinge N′ portion-optional N′ peptide linker-cytokine moiety-optional C′ peptide linker-hinge C′ portion-CH2; (11) ligand-optional linker-hinge N′ portion-optional N′ peptide linker-cytokine moiety-optional C′ peptide linker-hinge C′ portion-CH2-CH3; (12) ligand-optional linker-hinge N′ portion-optional N′ peptide linker-cytokine moiety-optional C′ peptide linker-hinge C′ portion-CH2; (13) receptor-optional linker-hinge N′ portion-optional N′ peptide linker-cytokine moiety-optional C′ peptide linker-hinge C′ portion-CH2-CH3; or (14) receptor-optional linker-hinge N′ portion-optional N′ peptide linker-cytokine moiety-optional C′ peptide linker-hinge C′ portion-CH2.

### Fc Domains

The immunocytokine descried herein comprises an Fc domain or portion thereof at the C-terminus. Fc domain comprises a CH2 domain and a CH3 domain. In some embodiments, the Fc domain portion comprises (consists essentially of or consists of) a CH2 domain. In some embodiments, the Fc domain portion comprises (consists essentially of or consists of) a CH3 domain.

In some embodiments, the Fc domain is derived from any of IgA, IgD, IgE, IgG, and IgM, and subtypes thereof. In some embodiments, the Fc domain comprises CH2 and CH3. In some embodiments, the Fc domain is derived from an IgG (e.g., IgG1, IgG2, IgG3, or IgG4). In some embodiments, the Fc domain is derived from a human IgG. In some embodiments, the Fc domain is derived from a human IgG1 or human IgG4. In some embodiments, the two subunits of the Fc domain dimerize via one or more (e.g., 1, 2, 3, 4, or more) disulfide bonds. In some embodiments, each subunit of the Fc domain comprises a full-length Fc sequence. In some embodiments, each subunit of the Fc domain comprises an N-terminus truncated Fc sequence. In some embodiments, the Fc domain is truncated at the N-terminus, e.g., lacks the first 1, 2, 3, 4, 5, 6, 7, 8, 9, or 10 amino acids of a complete immunoglobulin Fc domain. In some embodiments, the Fc domain comprises the amino acid sequence of any of SEQ ID NOs: 60-66.

Via the Fc domain, immunocytokines can activate complement and interact with Fc receptors. This inherent immunoglobulin feature has been viewed unfavorably because immunocytokines may be targeted to cells expressing Fc receptors rather than the preferred antigen-bearing cells. Moreover, the simultaneous activation of cytokine receptors and Fc receptor signaling pathways leading to cytokine release, especially in combination with the long half-life of immunoglobulin fusion proteins, make their application in a therapeutic setting difficult due to systemic toxicity. Thus in some embodiments, the Fc domain is engineered to have altered binding to an Fc receptor (FcR), specifically altered binding to an Fcγ receptor, and/or altered effector function, such as altered (e.g., reduced or eliminated) antibody-dependent cell-mediated cytotoxicity (ADCC), Antibody-dependent Cellular Phagocytosis (ADCP), and/or Complement-dependent cytotoxicity (CDC).

Although the presence of an Fc domain is essential for prolonging the half-life of the immunocytokine, in some situations it will be beneficial to eliminate effector functions associated with engagement of Fc receptors by the Fc domain. Hence, in some embodiments the altered binding to an Fc receptor and/or effector function is reduced binding and/or effector function. In some embodiments, the Fc domain comprises one or more amino acid mutation that reduces the binding of the Fc domain to an Fc receptor, particularly an Fcγ receptor (responsible for ADCC).

Preferably, such an amino acid mutation does not reduce binding to FcRn receptors (responsible for half-life). In some embodiments, the Fc domain subunit comprises the amino acid sequence of SEQ ID NO: 64 or 65. In some embodiments, the Fc domain is derived from human IgG1 and comprises the amino acid substitution N295A. In some embodiments, the Fc domain is derived from human IgG4 and comprises the amino acid substitutions S228P and L235E at the hinge region. In some embodiments, the Fc domain is derived from human IgG1 and comprises the amino acid substitutions L234A and L235A (“LALA”) at the hinge region. In some embodiments, the Fc domain is derived from human IgG1 and comprises the amino acid substitutions L234A and L235A at the hinge region, and P329G, e.g., in each of its subunits. See, e.g., Lo M. et al. J Biol Chem. 2017 Mar. 3; 292(9):3900-3908; Schlothauer T. et al. Protein Eng Des Sel. 2016 October; 29(10):457-466.

In some embodiments, the Fc domain (e.g., human IgG1) is mutated to remove one or more effector functions such as ADCC, ADCP, or CDC, namely, an “effectorless” or “almost effectorless” Fc domain. For example, in some embodiments, the Fc domain is an effectorless IgG1 Fc comprising one or more of the following mutations (such as in each of its subunits): L234A, L235E, G237A, A330S, and P331S. The combinations of K322A, L234A, and L235A in IgG1 are sufficient to almost completely abolish FcγR and Clq binding (Hezareh et al. J Virol 75, 12161-12168, 2001). MedImmune identified that a set of three mutations L234F/L235E/P331S have a very similar effect (Oganesyan et al., Acta Crystallographica 64, 700-704, 2008). In some embodiments, the Fc domain comprises a modification of the glycosylation on N297 of the IgG1 Fc domain, which is known to be required for optimal FcR interaction. The Fc domain modification can be any suitable IgG Fc engineering mentioned in Wang et al. (“IgG Fc engineering to modulate antibody effector functions,” Protein Cell. 2018 January; 9(1): 63-73), the content of which is incorporated herein by reference in its entirety.

In some embodiments, the Fc domain comprises two identical polypeptide chains (identical Fc subunits). Such Fc domains are herein also referred to as “homodimeric Fc domains.” In some embodiments, each subunit of the homodimeric Fc domain comprises the amino acid sequence of any of SEQ ID NOs: 60, and 63-66.

In some embodiments, the Fc domain comprises a modification promoting heterodimerization of two non-identical polypeptide chains. Such Fc domains are herein also referred to as “heterodimeric Fc domains.” In some embodiments, the Fc domain comprises a knob-into-hole (KIH) modification, comprising a knob modification in one of the subunits of the Fc domain and a hole modification in the other one of the two subunits of the Fc domain. Any suitable knob-into-hole modifications can be applied to the immunocytokine described herein, such as amino acid changes of T22>Y (creating the knob) in strand B of the first CH3 domain and Y86>T (creating the hole) in strand E of the partner CH3 domain. Also see US20200087414, the content of which is incorporated herein by reference in its entirety. In some embodiments, one subunit of the Fc domain comprises one or more of T350V, L351Y, S400E, F405A, and Y407V mutations relative to a wildtype human IgG1 Fc comprising the sequence of SEQ ID NO: 60, and the other subunit of the Fc domain comprises one or more of T350V, T366L, N390R, K392M, T394W mutations relative to a wildtype human IgG1 Fc comprising the sequence of SEQ ID NO: 60. In some embodiments, one subunit of the Fc domain comprises the sequence of SEQ ID NO: 61, and the other subunit of the Fc domain comprises the sequence of SEQ ID NO: 62.

In some embodiments, the Fc domain is a single chain Fc domain as described in WO2017134140, the content of which is incorporated herein by reference in its entirety.

### Linkers

In some embodiments, within the immunocytokine described herein, between the two or more antigen-binding fragments connected in tandem, the antigen-binding fragment (e.g., ligand, receptor, VHH, scFv, or Fab) and the cytokine moiety, the CH1 domain and the cytokine moiety, the two or more cytokine moieties connected in tandem, the two or more subunits of a cytokine or variant thereof connected in tandem, the cytokine moiety and the Fc domain subunit or portion thereof, the hinge region and the CH1 domain, the hinge region and the CH2 domain, the hinge region and the cytokine moiety, the Fc domain subunit or portion thereof and the antigen-binding fragment, and/or the CH1 domain and the Fc domain subunit or portion thereof, are connected via one or more optional linkers (e.g., peptide linker, non-peptide linker). In some embodiments, the one or more linkers are the same. In some embodiments, the one or more linkers are different (e.g., different from each other). In some embodiments, the one or more linkers are flexible linkers. In some embodiments, the one or more linkers are stable linkers. In some embodiments, some of the linkers are flexible, while others are stable. In general, a linker does not affect or significantly affect the proper fold and conformation formed by the configuration of the immunocytokine. Preferably, the linker confers flexibility and spatial space for each portion of the immunocytokine, such as allows target antigen-antigen binding fragment binding, allows ligand-receptor binding, masking cytokine activity in the absence of target antigen-antibody (or ligand-receptor) binding, while unmasking cytokine activity in the presence of target antigen-antibody (or ligand-receptor) binding, providing flexibility and/or sufficient space between two cytokine subunits or two cytokine moieties to ensure proper cytokine activity (binding affinity and/or bioactivity), etc.

The linkers can be peptide linkers of any length. In some embodiments, the peptide linker is from about 1 amino acid (aa) to about 10 aa long, from about 2 aa to about 15 aa long, from about 3 aa to about 12 aa long, from about 4 aa to about 10 aa long, from about 5 aa to about 9 aa long, from about 6 aa to about 8 aa long, from about 1 amino acid to about 20 aa long, from about 21 aa to about 30 aa long, from about 1 amino acid to about 30 aa long, from about 2 aa to about 20 aa long, from about 10 aa to about 30 aa long, from about 1 amino acid to about 50 aa long, from about 2 aa to about 19 aa long, from about 2 aa to about 18 aa long, from about 2 aa to about 17 aa long, from about 2 aa to about 16 aa long, from about 2 aa to about 10 aa long, from about 2 aa to about 14 aa long, from about 2 aa to about 13 aa long, from about 2 aa to about 12 aa long, from about 2 aa to about 11 aa long, from about 2 aa to about 9 aa long, from about 2 aa to about 8 aa long, from about 2 aa to about 7 aa long, from about 2 aa to about 6 aa long, from about 2 aa to about 5 aa long, or from about 6 aa to about 30 aa long. In some embodiments, the peptide linker is about any of 1, 2, 3, 4, 5, 6, 7, 8, 9, 10, 11, 12, 13, 14, 15, 16, 17, 18, 19 or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. In some embodiments, the linker is about 10 to about 20 amino acids in length.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge or portion thereof. In some embodiments, the peptide linker is a mutated human IgG1, IgG2, IgG3, or IgG4 hinge or portion thereof. In some embodiments, the linker is a flexible linker. Exemplary flexible linkers include, but are not limited to, glycine polymers (G)ₙ(SEQ ID NO: 194), glycine-serine polymers (including, for example, (GS)ₙ(SEQ ID NO: 195), (GGS)ₙ(SEQ ID NO: 196), (GGGS)ₙ(SEQ ID NO: 197), (GGS)ₙ(GGGS)ₙ(SEQ ID NO: 198), (GSGGS)ₙ(SEQ ID NO: 199), (GGSGS)ₙ(SEQ ID NO: 200), or (GGGGS)ₙ(SEQ ID NO: 201), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev.

Computational Chem. 11 173-142 (1992)). Exemplary flexible linkers include, but are not limited to GG (SEQ ID NO: 202), GSG (SEQ ID NO: 203), GGSG (SEQ ID NO: 204), GGSGG (SEQ ID NO: 205), GSGGGGG (SEQ ID NO: 206), GSGSG (SEQ ID NO: 207), GSGGG (SEQ ID NO: 208), GGGSG (SEQ ID NO: 209), GSSSG (SEQ ID NO: 210), GGSGGS (SEQ ID NO: 211), SGGGGS (SEQ ID NO: 212), GGGGS (SEQ ID NO: 213), (GA)ₙ(SEQ ID NO: 214, n is an integer of at least 1), GRAGGGGAGGGG (SEQ ID NO: 215), GRAGGG (SEQ ID NO: 216), GSGGGSGGGGSGGGGS (SEQ ID NO: 217), GGGSGGGGSGGGGS (SEQ ID NO: 218), GGGSGGSGGS (SEQ ID NO: 219), GGSGGSGGSGGSGGG (SEQ ID NO: 220), GGSGGSGGGGSGGGGS (SEQ ID NO: 221), GGSGGSGGSGGSGGSGGS (SEQ ID NO: 222), GGGGSGGGGSGGGGS (SEQ ID NO: 229), GGGGGGSGGGGSGGGGSA (SEQ ID NO: 223), GSGGGSGGGGSGGGGSGGGGS (SEQ ID NO: 224), KTGGGSGGGS (SEQ ID NO: 225), GGPGGGGSGGGSGGGGS (SEQ ID NO: 226), GGGSGGGGSGGGGSGGGGS (SEQ ID NO: 227), GGGGSGGGGSGGGGSGGGGSG (SEQ ID NO: 228), and the like. In some embodiments, the linker comprises the sequence of ASTKGP (SEQ ID NO: 230). The ordinarily skilled artisan will recognize that design of an immunocytokine can include linkers that are all or partially flexible, such that the linker can include a flexible linker portion as well as one or more portions that confer less flexible structure to provide a desired immunocytokine structure and function (e.g., masking cytokine activity in the absence of target antigen-antibody binding, while unmasking cytokine activity in the presence of target antigen-antibody binding; or providing flexibility and/or sufficient space between two cytokine subunits to ensure proper cytokine activity (binding affinity and/or bioactivity)). In some embodiments, the peptide linker is enriched in serine-glycine. In some embodiments, the peptide linker comprises the amino acid sequence of any one of SEQ ID NOs: 194-242. In some embodiments, the peptide linker comprises (or consists essentially of, or consists of) the amino acid sequence of any of SEQ ID NOs: 227-229. In some embodiments, the cytokine moiety described herein comprises two cytokine subunits (wildtype or mutant) connected by a linker, such as a linker comprising the sequence of any of SEQ ID NOs: 227-229.

In some embodiments, the linker is a stable linker (e.g., not cleavable by protease, especially MMPs).

Any one or all of the linkers described herein can be accomplished by any chemical reaction that will connect the antigen-binding fragment (e.g., ligand, receptor, VHH, scFv, or Fab) and the hinge region, the hinge region and the cytokine moiety, the C_{H}1 domain and the cytokine moiety, the cytokine moieties connected in tandem, the cytokine moiety and the Fc domain subunit, between two cytokine subunits, between antigen-binding fragments, between antigen binding fragment and the cytokine moiety, between antigen-binding fragment and Fc domain subunit or portion thereof, and/or the C_{H}1 domain and the Fc domain subunit or portion thereof, so long as the components or fragments retain their respective activities, i.e. binding to cytokine receptor, binding to target antigen(s), binding to ligand or receptor, binding to FcR, or ADCC. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester)). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above can contain components that have different attributes, thus leading to immunocytokines with differing physio-chemical properties. For example, sulfo-NHS esters of alkyl carboxylates are more stable than sulfo-NHS esters of aromatic carboxylates.

NHS-ester containing linkers are less soluble than sulfo-NHS esters. Further, the linker SIPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodiimide couplings. Carbodiimide couplings (such as EDC) when used in conjunction with sulfo-NHS, forms esters that are more resistant to hydrolysis than the carbodiimide coupling reaction alone.

Other linker considerations include the effect on physical or pharmacokinetic properties of the resulting immunocytokine, such as solubility, lipophilicity, hydrophilicity, hydrophobicity, stability (more or less stable as well as planned degradation), rigidity, flexibility, immunogenicity, modulation of cytokine moiety/cytokine receptor binding, modulation of antigen-binding domain/target antigen binding, modulation of ligand-receptor binding, the ability to be incorporated into a micelle or liposome, and the like.

### Immunocytokine Variants

Glycosylation Variants

In some embodiments, the immunocytokine is altered to increase or decrease the extent to which the construct is glycosylated. Addition or deletion of glycosylation sites to an Fc domain may be conveniently accomplished by altering the amino acid sequence such that one or more glycosylation sites is created or removed.

Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc domain may be made in order to create certain improved properties.

In some embodiments, the immunocytokine described herein is provided having a carbohydrate structure that lacks fucose attached (directly or indirectly) to the Fc domain. For example, the amount of fucose in such immunocytokine may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function.

See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Effector Function Variants

In some embodiments, the present application contemplates an immunocytokine that possesses some but not all Fc effector functions, which makes it a desirable candidate for applications in which the half-life of the immunocytokine in vivo is important yet certain effector functions (such as CDC and ADCC) are unnecessary or deleterious. Some of the Fc domain variants have been discussed above. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad.

Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M.S. and M.J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Fc domains with reduced effector function include those with substitution of one or more of Fc region residues 238, 265, 269, 270, 297, 327 and 329 (U.S. Pat. No. 6,737,056). Such Fc mutants include substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9(2): 6591-6604 (2001)). In some embodiments, alterations are made in the Fc domain that result in altered (i.e., either improved or diminished) Clq binding and/or CDC, e.g., as described in U.S. Pat. No. 6,194,551, WO 99/51642, and Idusogie et al. J. Immunol. 164: 4178-4184 (2000).

In some embodiments, the Fc domain comprises one or more amino acid substitutions, which increase half-life and/or improve binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc domain variants.

Cysteine Engineered Variants

In some embodiments, it may be desirable to create cysteine-engineered immunocytokines, e.g., “thioMAbs,” in which one or more residues of an immunocytokine are substituted with cysteine residues. In particular, embodiments, the substituted residues occur at accessible sites of the immunocytokine. By substituting those residues with cysteine, reactive thiol groups are thereby positioned at accessible sites of the immunocytokine and may be used to conjugate the immunocytokine to other moieties, such as drug moieties or linker-drug moieties, to create an immunocytokine-conjugate. In some embodiments, any one or more of the following residues may be substituted with cysteine: A1 18 (EU numbering) of the heavy chain; and S400 (EU numbering) of the heavy chain Fc domain. Cysteine engineered immunocytokines may be generated as described, e.g., in U.S. Pat. No. 7,521,541.

Immunocytokine Derivatives

In some embodiments, immunocytokines provided herein may further comprise an additional therapeutic compound, such as any therapeutic compounds known in the art. For example, the parental antibody in some embodiments can be an antibody drug conjugate (ADC).

See, e.g., any ADC described in Shim H. (Biomolecules. 2020 March; 10(3): 360), and Diamantis N. and Banerji U. Br J Cancer. 2016; 114(4): 362-367, the contents of which are incorporated herein by reference in their entirety. In some embodiments, the therapeutic compound is conjugated to the Fc domain or portion thereof. In some embodiments, the therapeutic compound is a cytotoxic agent, a chemotherapeutic agent, a growth inhibitory agent, or an antibiotic.

In some embodiments, the immunocytokine further comprises a label selected from the group consisting of a chromophore, a fluorophore (e.g., coumarin, a xanthene, a cyanine, a pyrene, a borapolyazaindacene, an oxazine, and derivatives thereof), a fluorescent protein (e.g., GFP, phycobiliproteins, and derivatives thereof), a phosphorescent dye (e.g., dioxetanes, xanthene, or carbocyanine dyes, lanthanide chelates), a tandem dye (e.g., cyanine-phycobiliprotein derivative and xanthene-phycobiliprotein derivative), a particle (e.g., gold clusters, colloidal gold, microspheres, quantum dots), a hapten, an enzyme (e.g., peroxidase, a phosphatase, a glycosidase, a luciferase), and a radioisotope (e.g., ¹²⁵, ³H, ⁴C, ³²P)

### III. Vectors Encoding Immunocytokines

The present invention also provides isolated nucleic acids encoding any of the immunocytokines described herein (e.g., IL-2/anti-HER2 immunocytokine, IL-2/anti-CD3 immunocytokine, IL-2/anti-PD-1 immunocytokine, IL-2/anti-CD4 immunocytokine, IL-2/anti-CD8 immunocytokine, IL-2/anti-CTLA-4 immunocytokine, IL-2/anti-PD-L1 immunocytokine, IL-2/anti-CD25 immunocytokine, IL-2/PD-L2-Fc immunocytokine, IL-12/anti-HER2 immunocytokine, IL-12/anti-CD3 immunocytokine, IL-12/anti-PD-1 immunocytokine, IL-12/anti-CD4 immunocytokine, IL-12/anti-CD8 immunocytokine, IL-12/anti-CTLA-4 immunocytokine, IL-12/anti-PD-L1 immunocytokine, IL-12/anti-CD25 immunocytokine, IL-12/PD-L2-Fc immunocytokine, IL-23/anti-HER2 immunocytokine, IL-23/anti-CD3 immunocytokine, IL-23/anti-PD-1 immunocytokine, IL-23/anti-CD4 immunocytokine, IL-23/anti-CD8 immunocytokine, IL-23/anti-CTLA-4 immunocytokine, IL-23/anti-PD-L1 immunocytokine, IL-23/anti-CD25 immunocytokine, IL-23/PD-L2-Fc immunocytokine, IL-10/anti-HER2 immunocytokine, IL-10/anti-CD3 immunocytokine, IL-10/anti-PD-1 immunocytokine, IL-10/anti-CD4 immunocytokine, IL-10/anti-CD8 immunocytokine, IL-10/anti-CTLA-4 immunocytokine, IL-10/anti-PD-L1 immunocytokine, IL-10/anti-CD25 immunocytokine, IL-10/PD-L2-Fc immunocytokine, IFN-γ/anti-HER2 immunocytokine, IFN-γ/anti-CD3 immunocytokine, IFN-γ/anti-PD-1 immunocytokine, IFN-γ/anti-CD4 immunocytokine, IFN-γ/anti-CD8 immunocytokine, IFN-γ/anti-CTLA-4 immunocytokine, IFN-γ/anti-PD-L1 immunocytokine, IFN-γ/anti-CD25 immunocytokine, IFN-γ/PD-L2-Fc immunocytokine, IFN-α/anti-HER2 immunocytokine, IFN-α/anti-CD3 immunocytokine, IFN-α/anti-PD-1 immunocytokine, IFN-α/anti-CD4 immunocytokine, IFN-α/anti-CD8 immunocytokine, IFN-α/anti-CTLA-4 immunocytokine, IFN-α/anti-PD-L1 immunocytokine, IFN-α/anti-CD25 immunocytokine, or IFN-α/PD-L2-Fc immunocytokine), vectors comprising nucleic acids encoding any of the immunocytokines described herein. Also provided are isolated host cells (e.g., CHO cells, HEK 293 cells, Hela cells, COS cells) comprising nucleic acids encoding any of the immunocytokines described herein, or vectors comprising nucleic acids encoding any of the immunocytokines described herein.

Thus in some embodiments, there is provided an isolated nucleic acid encoding an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand such as PD-L2, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided an isolated nucleic acid encoding an immunocytokine comprising: a) an antibody (e.g., full-length antibody, heavy chain only antibody, or antigen-binding fragment fused to an Fc domain subunit or portion thereof) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided an isolated nucleic acid encoding an immunocytokine comprising: a) an antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising from N-terminus to C-terminus: a VH domain, optionally a CH1 domain, the cytokine or variant thereof at a hinge region, a CH2 domain, and optionally a CH3 domain. In some embodiments, there is provided an isolated nucleic acid encoding an immunocytokine comprising: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, each polypeptide of the immunocytokine is encoded by a single isolated nucleic acid. In some embodiments, one isolated nucleic acid encode two or more polypeptides of the immunocytokine.

In some embodiments, the vector comprising a nucleic acid encoding any of the immunocytokines described herein is suitable for replication and integration in eukaryotic cells, such as mammalian cells (e.g., CHO cells, HEK 293 cells, Hela cells, COS cells). In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunocytokine coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the immunocytokine-encoding nucleic acid(s) to the host cells. See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids or vectors encoding the immunocytokines described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the immunocytokines described herein from the population of host cells transfected through vectors (e.g., lentiviral vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some embodiments, the vector (e.g., viral vector) comprises any one of the nucleic acids encoding the immunocytokines described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

In some embodiments, the nucleic acid encoding the immunocytokines described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the nucleic acid encoding the immunocytokines described herein is operably linked to a CMV promoter.

In some embodiments, the nucleic acid encoding the immunocytokines described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of: inducer, irradiation (such as ionizing radiation, light), temperature (such as heat), redox state, and the activation state of the host cell. In some embodiments, the inducible promoter can be an NFAT promoter, a TETON® promoter, or an NFxB promoter. In some embodiments, the inducible promoter is a tet-inducible promoter.

In some embodiments, the vector comprises more than one nucleic acids encoding the immunocytokines described herein, e.g., different polypeptides of the immunocytokine. In some embodiments, each vector comprises 2 nucleic acids encoding 2 polypeptides of the immunocytokines described herein.

In some embodiments, the two or more nucleic acids encoding the immunocytokines described herein are operably regulated under the same promoter in the vector. In some embodiments, the two or more nucleic acids are linked in tandem via a linking sequence (e.g., IRES) or a nucleic acid sequence encoding a self-cleaving 2A peptide, such as P2A, T2A, E2A, F2A, BmCPV 2A, BmIFV 2A. In some embodiments, the nucleic acid encoding two or more polypeptides of the immunocytokines comprises linking sequence(s) (e.g., IRES) or nucleic acid sequence(s) encoding self-cleaving 2A peptide(s) (such as P2A, T2A, E2A, F2A, BmCPV 2A, BmIFV 2A) between the polypeptide encoding sequences. In some embodiments, the two or more nucleic acids encoding the immunocytokines described herein are operably regulated under separate promoters in the vector. In some embodiments, the promoters operably linked to each nucleic acid are different. In some embodiments, the promoters operably linked to each nucleic acid are the same. In some embodiments, the immunocytokine described herein is encoded by two or more vectors, e.g., each vector encodes one heavy chain (or one polypeptide comprising V_{H} and cytokine moiety) and one pairing light chain, or each vector encodes one polypeptide of the immunocytokine.

### IV. Methods of Preparation

Also provided are methods of preparing any of the immunocytokines described herein (e.g., IL-2/anti-HER2 immunocytokine, IL-2/anti-CD3 immunocytokine, IL-2/anti-PD-1 immunocytokine, IL-2/anti-CD4 immunocytokine, IL-2/anti-CD8 immunocytokine, IL-2/anti-CTLA-4 immunocytokine, IL-2/anti-PD-L1 immunocytokine, IL-2/anti-CD25 immunocytokine, IL-2/PD-L2-Fc immunocytokine, IL-12/anti-HER2 immunocytokine, IL-12/anti-CD3 immunocytokine, IL-12/anti-PD-1 immunocytokine, IL-12/anti-CD4 immunocytokine, IL-12/anti-CD8 immunocytokine, IL-12/anti-CTLA-4 immunocytokine, IL-12/anti-PD-L1 immunocytokine, IL-12/anti-CD25 immunocytokine, IL-12/PD-L2-Fc immunocytokine, IL-23/anti-HER2 immunocytokine, IL-23/anti-CD3 immunocytokine, IL-23/anti-PD-1 immunocytokine, IL-23/anti-CD4 immunocytokine, IL-23/anti-CD8 immunocytokine, IL-23/anti-CTLA-4 immunocytokine, IL-23/anti-PD-L1 immunocytokine, IL-23/anti-CD25 immunocytokine, IL-23/PD-L2-Fc immunocytokine, IL-10/anti-HER2 immunocytokine, IL-10/anti-CD3 immunocytokine, IL-10/anti-PD-1 immunocytokine, IL-10/anti-CD4 immunocytokine, IL-10/anti-CD8 immunocytokine, IL-10/anti-CTLA-4 immunocytokine, IL-10/anti-PD-L1 immunocytokine, IL-10/anti-CD25 immunocytokine, IL-10/PD-L2-Fc immunocytokine, IFN-γ/anti-HER2 immunocytokine, IFN-γ/anti-CD3 immunocytokine, IFN-γ/anti-PD-1 immunocytokine, IFN-γ/anti-CD4 immunocytokine, IFN-γ/anti-CD8 immunocytokine, IFN-γ/anti-CTLA-4 immunocytokine, IFN-γ/anti-PD-L1 immunocytokine, IFN-γ/anti-CD25 immunocytokine, IFN-γ/PD-L2-Fc immunocytokine, IFN-α/anti-HER2 immunocytokine, IFN-α/anti-CD3 immunocytokine, IFN-α/anti-PD-1 immunocytokine, IFN-α/anti-CD4 immunocytokine, IFN-α/anti-CD8 immunocytokine, IFN-α/anti-CTLA-4 immunocytokine, IFN-α/anti-PD-L1 immunocytokine, IFN-α/anti-CD25 immunocytokine, or IFN-α/PD-L2-Fc immunocytokine). Thus, in some embodiments, there is provided a method of producing an immunocytokine, comprising: (a) culturing a host cell (e.g., CHO cell, HEK 293 cell, Hela cell, or COS cell) comprising any of the nucleic acids or vectors encoding the immunocytokines described herein under a condition effective to express the encoded immunocytokine; and (b) obtaining the expressed immunocytokine from said host cell. In some embodiments, the method of step (a) further comprises producing a host cell comprising the nucleic acid or vector encoding the immunocytokine described herein. The immunocytokine described herein may be prepared using any methods known in the art or as described herein. Also see Examples 1, 4, 5, 7, 9, 10, and 12 for exemplary methods. In some embodiments, the immunocytokines is expressed with eukaryotic cells, such as mammalian cells. In some embodiments, the immunocytokines is expressed with prokaryotic cells.

### 1. Recombinant Production in Prokaryotic Cells

### a) Vector Construction

Polynucleic acid sequences encoding the immunocytokines of the present application can be obtained using standard recombinant techniques. Desired polynucleic acid sequences may be isolated and sequenced from antibody or immunocytokine producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector, which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the polypeptide by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the —galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e., cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In some embodiments of the present application, the signal sequences used in both cistrons of the expression system are STII signal sequences or variants thereof.

In some embodiments, the production of the immunocytokine according to the present application can occur in the cytoplasm of the host cell, and therefore does not require the presence of secretion signal sequences within each cistron. In some embodiments, polypeptide components are expressed, folded, and assembled to form an immunocytokine (or portion of the immunocytokine) within the cytoplasm. Certain host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

The present invention provides an expression system in which the quantitative ratio of expressed polypeptide components can be modulated in order to maximize the yield of secreted and properly assembled immunocytokines of the present application. Such modulation is accomplished at least in part by simultaneously modulating translational strengths for the polypeptide components. One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleic acid sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired protein products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the present application.

### b) Prokaryotic Host Cells

Prokaryotic host cells suitable for expressing the immunocytokines of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the immunocytokines of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed immunocytokines of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the immunocytokines of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD, or DsbG) or FkpA (a peptidylprolyl cis, trans-isomerase with chaperone activity) can be used to co-transform the host prokaryotic cells.

The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001)Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the immunocytokines of the present application.

### d) Protein Purification

The immunocytokines produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the immunocytokines comprising an Fc region of the present application. Protein A is a 42 kDa surface protein from Staphylococcus aureas which binds with a high affinity to Fc-containing constructs, e.g., antigen-binding fragment-hinge-Fc fusion proteins, antibodies, or immunocytokines described herein. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the immunocytokines of interest are recovered from the solid phase by elution.

### 2. Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

### a) Signal Sequence Component

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the immunocytokines of the present application.

b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the immunocytokines of the present application, such as DHFR, thymidine kinase, met allothionein-I and —II, preferably primate met allothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences.

Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors. Also see “Promoters” subsection under “III. Vectors encoding immunocytokines” above.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the immunocytokines of the present application by higher eukaryotes is often increased by inserting an enhancer sequence into the vector. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for immunocytokine production and cultured in conventional nutrient media modified as appropriate for inducing promoters, selecting transformants, or amplifying the genes encoding the desired sequences.

### h) Culturing the Host Cells

The host cells used to produce the immunocytokines of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### i) Protein Purification

When using recombinant techniques, the immunocytokine can be produced intracellularly, in the periplasmic space, or directly secreted into the medium. If the immunocytokine is produced intracellularly, as a first step, the particulate debris, either host cells or lysed fragments, are removed, for example, by centrifugation or ultrafiltration. Carter et al., Bio Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the immunocytokine is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the immunocytokine. Protein A can be used to purify the immunocytokines, antigen-binding fragment-Fc fusion proteins, or antibodies that are based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly(styrene-divinyl) benzene allow for faster flow rates and shorter processing times than can be achieved with agarose.

Where the immunocytokine comprises a C_{H}3 domain, the Bakerbond ABXTMresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the immunocytokine to be recovered.

Following any preliminary purification step(s), the mixture comprising the immunocytokine of interest and contaminants may be subjected to low pH hydrophobic interaction chromatography using an elution buffer at a pH between about 2.5-4.5, preferably performed at low salt concentrations (e.g., from about 0-0.25M salt).

### V. Pharmaceutical Compositions

Further provided are pharmaceutical compositions comprising any of the immunocytokines described herein (e.g., IL-2/anti-HER2 immunocytokine, IL-2/anti-CD3 immunocytokine, IL-2/anti-PD-1 immunocytokine, IL-2/anti-CD4 immunocytokine, IL-2/anti-CD8 immunocytokine, IL-2/anti-CTLA-4 immunocytokine, IL-2/anti-PD-L1 immunocytokine, IL-2/anti-CD25 immunocytokine, IL-2/PD-L2-Fc immunocytokine, IL-12/anti-HER2 immunocytokine, IL-12/anti-CD3 immunocytokine, IL-12/anti-PD-1 immunocytokine, IL-12/anti-CD4 immunocytokine, IL-12/anti-CD8 immunocytokine, IL-12/anti-CTLA-4 immunocytokine, IL-12/anti-PD-L1 immunocytokine, IL-12/anti-CD25 immunocytokine, IL-12/PD-L2-Fc immunocytokine, IL-23/anti-HER2 immunocytokine, IL-23/anti-CD3 immunocytokine, IL-23/anti-PD-1 immunocytokine, IL-23/anti-CD4 immunocytokine, IL-23/anti-CD8 immunocytokine, IL-23/anti-CTLA-4 immunocytokine, IL-23/anti-PD-L1 immunocytokine, IL-23/anti-CD25 immunocytokine, IL-23/PD-L2-Fc immunocytokine, IL-10/anti-HER2 immunocytokine, IL-10/anti-CD3 immunocytokine, IL-10/anti-PD-1 immunocytokine, IL-10/anti-CD4 immunocytokine, IL-10/anti-CD8 immunocytokine, IL-10/anti-CTLA-4 immunocytokine, IL-10/anti-PD-L1 immunocytokine, IL-10/anti-CD25 immunocytokine, IL-10/PD-L2-Fc immunocytokine, IFN-γ/anti-HER2 immunocytokine, IFN-γ/anti-CD3 immunocytokine, IFN-γ/anti-PD-1 immunocytokine, IFN-γ/anti-CD4 immunocytokine, IFN-γ/anti-CD8 immunocytokine, IFN-γ/anti-CTLA-4 immunocytokine, IFN-γ/anti-PD-L1 immunocytokine, IFN-γ/anti-CD25 immunocytokine, IFN-γ/PD-L2-Fc immunocytokine, IFN-α/anti-HER2 immunocytokine, IFN-α/anti-CD3 immunocytokine, IFN-α/anti-PD-1 immunocytokine, IFN-α/anti-CD4 immunocytokine, IFN-α/anti-CD8 immunocytokine, IFN-α/anti-CTLA-4 immunocytokine, IFN-α/anti-PD-L1 immunocytokine, IFN-α/anti-CD25 immunocytokine, or IFN-α/PD-L2-Fc immunocytokine), and optionally a pharmaceutically acceptable carrier. Pharmaceutical compositions can be prepared by mixing an immunocytokine described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Also provided are cancer vaccines comprising any of the immunocytokines described herein, or pharmaceutical compositions thereof.

A reconstituted formulation can be prepared by dissolving a lyophilized immunocytokine described herein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical composition comprises a homogeneous population of immunocytokines described herein. A homogeneous population means the immunocytokines are exactly the same to each other, e.g., same immunocytokine configuration, same cytokine moiety, same antigen-binding fragments (e.g., ligand, receptor, VHH, scFv, or Fab), same linker if any, same hinge region, and same Fc domain. In some embodiments, at least about 70% (such as at least about any of 75%, 80%, 85%, 90%, 95%, 96%, 97%, 98%, or 99%) of the immunocytokines in the pharmaceutical composition are homogeneous.

The pharmaceutical composition is preferably to be stable, in which the immunocytokine here essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of immunocytokines described herein may comprise less than about 10% (preferably less than about 5%) of the immunocytokines present as an aggregate in the formulation.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g., sodium chloride), stabilizers, metal complexes (e.g., Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™ polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). The addition of a preservative may, for example, facilitate the production of a multi-use (multiple dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the immunocytokines as well as to protect the immunocytokines against agitation-induced aggregation, which also permits the formulation to be exposed to shear surface stress without causing denaturation of the active immunocytokines. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 18th edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### VI. Methods of Treating Diseases or Directing Cytokine Activity

The immunocytokines described herein (e.g., IL-2/anti-HER2 immunocytokine, IL-2/anti-CD3 immunocytokine, IL-2/anti-PD-1 immunocytokine, IL-2/anti-CD4 immunocytokine, IL-2/anti-CD8 immunocytokine, IL-2/anti-CTLA-4 immunocytokine, IL-2/anti-PD-L1 immunocytokine, IL-2/anti-CD25 immunocytokine, IL-2/PD-L2-Fc immunocytokine, IL-12/anti-HER2 immunocytokine, IL-12/anti-CD3 immunocytokine, IL-12/anti-PD-1 immunocytokine, IL-12/anti-CD4 immunocytokine, IL-12/anti-CD8 immunocytokine, IL-12/anti-CTLA-4 immunocytokine, IL-12/anti-PD-L1 immunocytokine, IL-12/anti-CD25 immunocytokine, IL-12/PD-L2-Fc immunocytokine, IL-23/anti-HER2 immunocytokine, IL-23/anti-CD3 immunocytokine, IL-23/anti-PD-1 immunocytokine, IL-23/anti-CD4 immunocytokine, IL-23/anti-CD8 immunocytokine, IL-23/anti-CTLA-4 immunocytokine, IL-23/anti-PD-L1 immunocytokine, IL-23/anti-CD25 immunocytokine, IL-23/PD-L2-Fc immunocytokine, IL-10/anti-HER2 immunocytokine, IL-10/anti-CD3 immunocytokine, IL-10/anti-PD-1 immunocytokine, IL-10/anti-CD4 immunocytokine, IL-10/anti-CD8 immunocytokine, IL-10/anti-CTLA-4 immunocytokine, IL-10/anti-PD-L1 immunocytokine, IL-10/anti-CD25 immunocytokine, IL-10/PD-L2-Fc immunocytokine, IFN-γ/anti-HER2 immunocytokine, IFN-γ/anti-CD3 immunocytokine, IFN-γ/anti-PD-1 immunocytokine, IFN-γ/anti-CD4 immunocytokine, IFN-γ/anti-CD8 immunocytokine, IFN-γ/anti-CTLA-4 immunocytokine, IFN-γ/anti-PD-L1 immunocytokine, IFN-γ/anti-CD25 immunocytokine, IFN-γ/PD-L2-Fc immunocytokine, IFN-α/anti-HER2 immunocytokine, IFN-α/anti-CD3 immunocytokine, IFN-α/anti-PD-1 immunocytokine, IFN-α/anti-CD4 immunocytokine, IFN-α/anti-CD8 immunocytokine, IFN-α/anti-CTLA-4 immunocytokine, IFN-α/anti-PD-L1 immunocytokine, IFN-α/anti-CD25 immunocytokine, or IFN-α/PD-L2-Fc immunocytokine) and compositions (e.g., pharmaceutical compositions) thereof are useful for a variety of applications, such as in diagnosis, molecular assays, and therapy. In some embodiments, there is provided a method of treating a disease (e.g., cancer, infection such as viral infection, autoimmune disease, allergy, graft rejection, or GvHD) in an individual (e.g., human), comprising administering to the individual an effective amount of any of the immunocytokines described herein or pharmaceutical compositions thereof. In some embodiments, there is also provided a method of selectively activating the activity of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, HER2, PD-1, CD123, CD3, CD4, or CD8) in an individual (e.g., human), comprising administering to the individual an effective amount of any of the immunocytokines described herein or pharmaceutical compositions thereof, wherein the activity of the cytokine or variant thereof is selectively activated upon binding of the immunocytokine to the target antigen. In some embodiments, the individual has been previously treated with any of the immunocytokines described herein, or pharmaceutical composition thereof against a cancer, and the treatment comprises preventing the cancer recurrence in the individual. In some embodiments, the treatment comprises preventing a cancer in the individual, regardless if the individual has the cancer before or not, or has been treated by the immunocytokines described herein or not. In some embodiments, there is also provided a method of preventing a cancer in an individual, comprising administering to the individual an effective amount of any of the immunocytokines described herein, pharmaceutical composition or cancer vaccine thereof.

Thus in some embodiments, there is provided a method of treating a disease (e.g., cancer, infection such as viral infection, autoimmune disease, allergy, GvHD, or graft rejection) in an individual (e.g., human), comprising administering to the individual an effective amount of an immunocytokine comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region; and optionally a pharmaceutical acceptable carrier. In some embodiments, there is provided a method of treating a disease (e.g., cancer, infection such as viral infection, autoimmune disease, allergy, GvHD, or graft rejection) in an individual (e.g., human), comprising administering to the individual an effective amount of an immunocytokine comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain; and optionally a pharmaceutical acceptable carrier. In some embodiments, there is provided a method of treating a disease (e.g., cancer, infection such as viral infection, autoimmune disease, allergy, GvHD, or graft rejection) in an individual (e.g., human), comprising administering to the individual an effective amount of an immunocytokine comprising: a) an antibody specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising from N-terminus to C-terminus: a V_{H} domain, optionally a C_{H}1 domain, the cytokine or variant thereof at a hinge region, a C_{H}2 domain, and optionally a C_{H}3 domain; and optionally a pharmaceutical acceptable carrier. In some embodiments, there is provided a method of treating a disease (e.g., cancer, infection such as viral infection, autoimmune disease, allergy, GvHD, or graft rejection) in an individual (e.g., human), comprising administering to the individual an effective amount of an immunocytokine comprising: a) a full-length antibody specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; and optionally a pharmaceutical acceptable carrier. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks.

In some embodiments, there is provided a method of treating a cancer in an individual (e.g., human), such as by selectively directing cytokine activity to an immune cell (e.g., T cell, B cell, monocyte, NK cell, myeloid DC cell, plasma DC cell, macrophage) in the individual, or by selectively directing cytokine activity to a cancer cell in the individual, comprising administering to the individual an effective amount of any of the IL-2/anti-HER2 immunocytokines, IL-10/anti-HER2 immunocytokines, IL-12/anti-HER2 immunocytokines, IL-23/anti-HER2 immunocytokines, IFN-γ/anti-HER2 immunocytokines, IFN-α/anti-HER2 immunocytokines, IL-2/anti-PD-1 immunocytokines, IL-10/anti-PD-1 immunocytokines, IL-12/anti-PD-1 immunocytokines, IL-23/anti-PD-1 immunocytokines, IFN-γ/anti-PD-1 immunocytokines, IFN-α/anti-PD-1 immunocytokines, IL-2/anti-PD-L1 immunocytokines, IL-10/anti-PD-L1 immunocytokines, IL-12/anti-PD-L1 immunocytokines, IL-23/anti-PD-L1 immunocytokines, IFN-γ/anti-PD-L1 immunocytokines, IFN-α/anti-PD-L1 immunocytokines, IL-2/anti-CTLA-4 immunocytokines, IL-10/anti-CTLA-4 immunocytokines, IL-12/anti-CTLA-4 immunocytokines, IL-23/anti-CTLA-4 immunocytokines, IFN-γ/anti-CTLA-4 immunocytokines, IFN-α/anti-CTLA-4 immunocytokines, IL-2/anti-CD3 immunocytokines, IL-10/anti-CD3 immunocytokines, IL-12/anti-CD3 immunocytokines, IL-23/anti-CD3 immunocytokines, IFN-γ/anti-CD3 immunocytokines, IFN-α/anti-CD3 immunocytokines, IL-2/anti-CD4 immunocytokines, IL-10/anti-CD4 immunocytokines, IL-12/anti-CD4 immunocytokines, IL-23/anti-CD4 immunocytokines, IFN-γ/anti-CD4 immunocytokines, IFN-α/anti-CD4 immunocytokines, IL-2/anti-CD8 immunocytokines, IL-10/anti-CD8 immunocytokines, IL-12/anti-CD8 immunocytokines, IL-23/anti-CD8 immunocytokines, IFN-γ/anti-CD8 immunocytokines, IFN-α/anti-CD8 immunocytokines, IL-2/anti-CD25 immunocytokines, IL-10/anti-CD25 immunocytokines, IL-12/anti-CD25 immunocytokines, IL-23/anti-CD25 immunocytokines, IFN-γ/anti-CD25 immunocytokines, IFN-α/anti-CD25 immunocytokines, IL-2/PD-L2-Fc immunocytokines, IL-10/PD-L2-Fc immunocytokines, IL-12/PD-L2-Fc immunocytokines, IL-23/PD-L2-Fc immunocytokines, IFN-γ/PD-L2-Fc immunocytokines, IFN-α/PD-L2-Fc immunocytokines described herein, or pharmaceutical composition thereof. In some embodiments, there is provided a method of treating a cancer in an individual (e.g., human), such as by selectively directing cytokine activity to an immune cell (e.g., T cell, B cell, monocyte, NK cell, myeloid DC cell, plasma DC cell, macrophage) in the individual, or by selectively directing cytokine activity to a cancer cell in the individual, comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating a cancer in an individual (e.g., human), such as by selectively directing cytokine activity to an immune cell (e.g., T cell, B cell, monocyte, NK cell, myeloid DC cell, plasma DC cell, macrophage) in the individual, or by selectively directing cytokine activity to a cancer cell in the individual, comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating a cancer in an individual (e.g., human), such as by selectively directing cytokine activity to an immune cell (e.g., T cell, B cell, monocyte, NK cell, myeloid DC cell, plasma DC cell, macrophage) in the individual, or by selectively directing cytokine activity to a cancer cell in the individual, comprising administering to the individual an effective amount of an immunocytokine (or pharmaceutical composition thereof) comprising: a) a full-length antibody specifically recognizing a target antigen (e.g., CTLA-4, PD-L1, PD-L2, CD25, HER2, PD-1, CD123, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer.

In some embodiments, there is provided a method of treating a cancer in an individual (e.g., human), or a method of selectively directing cytokine activity to a cancer cell in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen on the cancer cell (e.g., any tumor antigen such as HER2, any cancer cell surface ligand or receptor, or any immune checkpoint molecule expressed on cancer cells such as PD-L1 or PD-L2) of the cancer cell; and b) a cytokine (e.g., any cytokine described herein, such as IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating a cancer in an individual (e.g., human), or a method of selectively directing cytokine activity to a cancer cell in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen on the cancer cell (e.g., any tumor antigen such as HER2, any cancer cell surface ligand or receptor, or any immune checkpoint molecule expressed on cancer cells such as PD-L1 or PD-L2) of the cancer cell; and b) a cytokine (e.g., any cytokine described herein, such as IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating a cancer in an individual (e.g., human), or a method of selectively directing cytokine activity to a cancer cell in an individual (e.g., human), comprising administering to the individual an effective amount of an immunocytokine (or pharmaceutical composition thereof) selected from the group consisting of: IL-2/anti-HER2 immunocytokine, IL-10/anti-HER2 immunocytokine, IL-12/anti-HER2 immunocytokine, IL-23/anti-HER2 immunocytokine, IFN-γ/anti-HER2 immunocytokine, IFN-α/anti-HER2 immunocytokine, IL-2/anti-PD-L1 immunocytokine, IL-10/anti-PD-L1 immunocytokine, IL-12/anti-PD-L1 immunocytokine, IL-23/anti-PD-L1 immunocytokine, IFN-γ/anti-PD-L1 immunocytokine, and IFN-α/anti-PD-L1 immunocytokine. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer. In some embodiments, the cancer is HER2+ cancer, such as HER2+ breast cancer. In some embodiments, the cancer is PD-L1+ and/or PD-L2+ cancer.

In some embodiments, there is provided a method of treating a cancer in an individual (e.g., human), or a method of selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen on an immune cell (e.g., any immune cell surface antigen such as CD3, CD4, CD8, CD123, or CD25, or any immune checkpoint molecule expressed on immune cells such as PD-1 or CTLA-4); and b) a cytokine (e.g., any cytokine described herein, such as IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating a cancer in an individual (e.g., human), or a method of selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen on an immune cell (e.g., any immune cell surface antigen such as CD3, CD4, CD8, CD123, or CD25, or any immune checkpoint molecule expressed on immune cells such as PD-1 or CTLA-4); and b) a cytokine (e.g., any cytokine described herein, such as IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating a cancer in an individual (e.g., human), or a method of selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) selected from the group consisting of: IL-2/anti-PD-L1 immunocytokine, IL-10/anti-PD-L1 immunocytokine, IL-12/anti-PD-L1 immunocytokine, IL-23/anti-PD-L1 immunocytokine, IFN-γ/anti-PD-L1 immunocytokine, IFN-α/anti-PD-L1 immunocytokine, IL-2/anti-PD-1 immunocytokine, IL-10/anti-PD-1 immunocytokine, IL-12/anti-PD-1 immunocytokine, IL-23/anti-PD-1 immunocytokine, IFN-γ/anti-PD-1 immunocytokine, IFN-α/anti-PD-1 immunocytokine, IL-2/anti-CTLA-4 immunocytokine, IL-10/anti-CTLA-4 immunocytokine, IL-12/anti-CTLA-4 immunocytokine, IL-23/anti-CTLA-4 immunocytokine, IFN-γ/anti-CTLA-4 immunocytokine, IFN-α/anti-CTLA-4 immunocytokine, IL-2/anti-CD3 immunocytokine, IL-10/anti-CD3 immunocytokine, IL-12/anti-CD3 immunocytokine, IL-23/anti-CD3 immunocytokine, IFN-γ/anti-CD3 immunocytokine, IFN-α/anti-CD3 immunocytokine, IL-2/anti-CD4 immunocytokine, IL-10/anti-CD4 immunocytokine, IL-12/anti-CD4 immunocytokine, IL-23/anti-CD4 immunocytokine, IFN-γ/anti-CD4 immunocytokine, IFN-α/anti-CD4 immunocytokine, IL-2/anti-CD8 immunocytokine, IL-10/anti-CD8 immunocytokine, IL-12/anti-CD8 immunocytokine, IL-23/anti-CD8 immunocytokine, IFN-γ/anti-CD8 immunocytokine, IFN-α/anti-CD8 immunocytokine, IL-2/anti-CD25 immunocytokine, IL-10/anti-CD25 immunocytokine, IL-12/anti-CD25 immunocytokine, IL-23/anti-CD25 immunocytokine, IFN-γ/anti-CD25 immunocytokine, IFN-α/anti-CD25 immunocytokine, IL-2/PD-L2-Fc immunocytokine, IL-10/PD-L2-Fc immunocytokine, IL-12/PD-L2-Fc immunocytokine, IL-23/PD-L2-Fc immunocytokine, IFN-γ/PD-L2-Fc immunocytokine, and IFN-α/PD-L2-Fc immunocytokine. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer. In some embodiments, the cancer is PD-L1+ and/or PD-L2+ cancer.

In some embodiments, the method of treating cancer has one or more of the following biological activities: (1) killing cancer cells; (2) inhibiting proliferation of cancer cells; (3) inducing immune response in a tumor (e.g., inducing infiltration of immune effector cells to tumor site, inducing immune cell proliferation, differentiation and/or activation, and/or inducing pro-inflammatory cytokine secretion by immune cells); (4) reducing tumor size; (5) alleviating one or more symptoms in an individual having cancer; (6) inhibiting tumor metastasis; (7) prolonging survival; (8) prolonging time to cancer progression; and (9) preventing, inhibiting, or reducing the likelihood of the recurrence of a cancer. In some embodiments, the method of killing cancer cells mediated by the immunocytokine or pharmaceutical composition described herein can achieve a tumor cell death rate of at least about any of 10%, 20%, 30%, 40%, 50%, 60%, 70%, 80%, 90%, 95%, or more. In some embodiments, the method of reducing tumor size mediated by the immunocytokine or pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the immunocytokine or pharmaceutical composition described herein can inhibit at least about 10% (including for example at least about any of 20%, 30%, 40%, 60%, 70%, 80%, 90%, or 100%) of the metastasis. In some embodiments, the method of prolonging survival of an individual (e.g., human) mediated by the immunocytokine or pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the immunocytokine or pharmaceutical composition described herein can prolong the time to cancer progression by at least any of 1, 2, 3, 4, 5, 6, 7, 8, 9, 10, 11, or 12 weeks. In some embodiments, the method of inducing immune response to a tumor can increase, enhance, or stimulate an immune response or function in a subject. In some embodiments, the immune response or function is increased, enhanced, and/or stimulated by activating effector cells (e.g., T cells, e.g., CD8+ and/or CD4+ T cells), expanding (increasing) an effector cell population, and/or killing target cells (e.g., target tumor cells) in the subject. In some embodiments, the CD4 and/or CD8 T cells in the individual have increased or enhanced priming, activation, proliferation, cytokine release and/or cytolytic activity relative to prior to the administration of the immunocytokine or pharmaceutical composition described herein.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as surgery, radiation, chemotherapy, immunotherapy, hormone therapy, or a combination thereof. In some embodiments, the method is used to treat an individual who has previously been treated. In some embodiments, the cancer has been refractory to prior therapy. In some embodiments, the method is used to treat an individual who has not previously been treated.

In some embodiments, the cancer is partially resistant to immune checkpoint inhibitor monotherapy (e.g., partially resistant to anti-PD-1 or anti-PD-L1 antibody monotherapy treatment).

In some embodiments, the cancer is a HER2-positive cancer, and the method comprises administering to the individual (e.g., human) an effective amount of any suitable immunocytokines described herein, such as any of IL-2/anti-HER2 immunocytokine, IL-10/anti-HER2 immunocytokine, IL-23/anti-HER2 immunocytokine, IL-12/anti-HER2 immunocytokine, IFN-γ/anti-HER2 immunocytokine, IFN-α/anti-HER2 immunocytokine, IL-2/anti-PD-1 immunocytokine, IL-10/anti-PD-1 immunocytokine, IL-23/anti-PD-1 immunocytokine, IL-12/anti-PD-1 immunocytokine, IFN-γ/anti-PD-1 immunocytokine, IFN-α/anti-PD-1 immunocytokine, IL-2/anti-PD-L1 immunocytokine, IL-10/anti-PD-L1 immunocytokine, IL-12/anti-PD-L1 immunocytokine, IL-23/anti-PD-L1 immunocytokine, IFN-γ/anti-PD-L1 immunocytokine, IFN-α/anti-PD-L1 immunocytokine, IL-2/anti-CTLA-4 immunocytokine, IL-10/anti-CTLA-4 immunocytokine, IL-12/anti-CTLA-4 immunocytokine, IL-23/anti-CTLA-4 immunocytokine, IFN-γ/anti-CTLA-4 immunocytokine, IFN-α/anti-CTLA-4 immunocytokine, IL-2/anti-CD3 immunocytokine, IL-10/anti-CD3 immunocytokine, IL-12/anti-CD3 immunocytokine, IL-23/anti-CD3 immunocytokine, IFN-γ/anti-CD3 immunocytokine, IFN-α/anti-CD3 immunocytokine, IL-2/anti-CD4 immunocytokine, IL-10/anti-CD4 immunocytokine, IL-12/anti-CD4 immunocytokine, IL-23/anti-CD4 immunocytokine, IFN-γ/anti-CD4 immunocytokine, IFN-α/anti-CD4 immunocytokine, IL-2/anti-CD8 immunocytokine, IL-10/anti-CD8 immunocytokine, IL-12/anti-CD8 immunocytokine, IL-23/anti-CD8 immunocytokine, IFN-γ/anti-CD8 immunocytokine, IFN-α/anti-CD8 immunocytokine, IL-2/anti-CD25 immunocytokine, IL-10/anti-CD25 immunocytokine, IL-12/anti-CD25 immunocytokine, IL-23/anti-CD25 immunocytokine, IFN-γ/anti-CD25 immunocytokine, IFN-α/anti-CD25 immunocytokine, IL-2/PD-L2-Fc immunocytokine, IL-10/PD-L2-Fc immunocytokine, IL-12/PD-L2-Fc immunocytokine, IL-23/PD-L2-Fc immunocytokine, IFN-γ/PD-L2-Fc immunocytokine, IFN-α/PD-L2-Fc immunocytokine. In some embodiments, the HER2-positive cancer is breast cancer or gastric cancer. In some embodiments, the HER2-positive breast cancer is early-stage breast cancer. In some embodiments, the HER2-positive breast cancer is metastatic breast cancer. In some embodiments, the HER2-positive cancer is recurrent cancer. In some embodiments, the recurrent cancer is locally recurrent cancer. In some embodiments, the HER2-positive cancer is advanced cancer. In some embodiments, the HER2-positive cancer is non-resectable.

In some embodiments, the cancer is a PD-L1 expressing cancer, and the method comprises administering to the individual (e.g., human) an effective amount of any suitable immunocytokines described herein, such as any of IL-2/anti-HER2 immunocytokine, IL-10/anti-HER2 immunocytokine, IL-12/anti-HER2 immunocytokine, IL-23/anti-HER2 immunocytokine, IFN-γ/anti-HER2 immunocytokine, IFN-α/anti-HER2 immunocytokine, IL-2/anti-PD-1 immunocytokine, IL-10/anti-PD-1 immunocytokine, IL-12/anti-PD-1 immunocytokine, IL-23/anti-PD-1 immunocytokine, IFN-γ/anti-PD-1 immunocytokine, IFN-α/anti-PD-1 immunocytokine, IL-2/anti-PD-L1 immunocytokine, IL-10/anti-PD-L1 immunocytokine, IL-12/anti-PD-L1 immunocytokine, IL-23/anti-PD-L1 immunocytokine, IFN-γ/anti-PD-L1 immunocytokine, IFN-α/anti-PD-L1 immunocytokine, IL-2/anti-CTLA-4 immunocytokine, IL-10/anti-CTLA-4 immunocytokine, IL-12/anti-CTLA-4 immunocytokine, IL-23/anti-CTLA-4 immunocytokine, IFN-γ/anti-CTLA-4 immunocytokine, IFN-α/anti-CTLA-4 immunocytokine, IL-2/anti-CD3 immunocytokine, IL-10/anti-CD3 immunocytokine, IL-12/anti-CD3 immunocytokine, IL-23/anti-CD3 immunocytokine, IFN-γ/anti-CD3 immunocytokine, IFN-α/anti-CD3 immunocytokine, IL-2/anti-CD4 immunocytokine, IL-10/anti-CD4 immunocytokine, IL-12/anti-CD4 immunocytokine, IL-23/anti-CD4 immunocytokine, IFN-γ/anti-CD4 immunocytokine, IFN-α/anti-CD4 immunocytokine, IL-2/anti-CD8 immunocytokine, IL-10/anti-CD8 immunocytokine, IL-12/anti-CD8 immunocytokine, IL-23/anti-CD8 immunocytokine, IFN-γ/anti-CD8 immunocytokine, IFN-α/anti-CD8 immunocytokine, IL-2/anti-CD25 immunocytokine, IL-10/anti-CD25 immunocytokine, IL-12/anti-CD25 immunocytokine, IL-23/anti-CD25 immunocytokine, IFN-γ/anti-CD25 immunocytokine, IFN-α/anti-CD25 immunocytokine, IL-2/PD-L2-Fc immunocytokine, IL-10/PD-L2-Fc immunocytokine, IL-12/PD-L2-Fc immunocytokine, IL-23/PD-L2-Fc immunocytokine, IFN-γ/PD-L2-Fc immunocytokine, IFN-α/PD-L2-Fc immunocytokine. In some embodiments, the method is suitable for treating cancers with aberrant PD-1 or PD-L1/PD-L2 expression (e.g., HER2+ cancer), activity and/or signaling include, by way of non-limiting example, hematological cancer and/or solid tumors. Some cancers whose growth may be inhibited using the immunocytokines of the invention include cancers typically responsive to immunotherapy. Non-limiting examples of other cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, the invention includes refractory or recurrent malignancies whose growth may be inhibited using the immunocytokines of the invention. The present invention is also useful for treatment of metastatic cancers, especially metastatic cancers that express PD-L1 (Iwai et al. (2005) Int. Immunol. 17:133-144). In some embodiments, the cancer with aberrant PD-1 or PD-L1/PD-L2 expression, activity and/or signaling is partially resistant to PD-1 or PD-L1 blockade (e.g., partially resistant to anti-PD-1 antibody or anti-PD-L1 antibody treatment).

In some embodiments, the methods described herein are suitable for treating a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), cutaneous T-cell lymphoma (CTCL), cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the methods described herein are suitable for treating a hematologic cancer chosen from one or more of acute myeloid leukemia (AML), chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the methods described herein are for treating infection, e.g., fungal, viral, bacterial, protozoal, or other parasitic infection. The infection can be caused by any pathogen described above in the “Pathogen antigen” subsection. Thus in some embodiments, there is provided a method of treating an infection (e.g., viral, fungal, bacterial, protozoal, or other parasitic infection) in an individual (e.g., human), such as by selectively directing cytokine activity to an immune cell (e.g., T cell, B cell, monocyte, NK cell, myeloid DC cell, plasma DC cell, macrophage) in an individual (e.g., human), comprising administering to the individual an effective amount of any of the IL-2/anti-CD8 immunocytokines, IL-12/anti-CD8 immunocytokines, IL-10/anti-CD8 immunocytokines, IL-23/anti-CD8 immunocytokines, IFN-γ/anti-CD8 immunocytokines, IFN-α/anti-CD8 immunocytokines, IL-2/anti-CD3 immunocytokines, IL-10/anti-CD3 immunocytokines, IL-12/anti-CD3 immunocytokines, IL-23/anti-CD3 immunocytokines, IFN-γ/anti-CD3 immunocytokines, IFN-α/anti-CD3 immunocytokines, IL-2/anti-CD4 immunocytokines, IL-10/anti-CD4 immunocytokines, IL-12/anti-CD4 immunocytokines, IL-23/anti-CD4 immunocytokines, IFN-γ/anti-CD4 immunocytokines, IFN-α/anti-CD4 immunocytokines, IL-2/anti-CD25 immunocytokines, IL-10/anti-CD25 immunocytokines, IL-12/anti-CD25 immunocytokines, IL-23/anti-CD25 immunocytokines, IFN-γ/anti-CD25 immunocytokines, and IFN-α/anti-CD25 immunocytokines described herein, or pharmaceutical composition thereof. In some embodiments, there is provided a method of treating an infection (e.g., viral, fungal, bacterial, protozoal, or other parasitic infection) in an individual (e.g., human), such as by selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CD123, CTLA-4, PD-L1, PD-L2, CD25, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating an infection (e.g., viral, fungal, bacterial, protozoal, or other parasitic infection) in an individual (e.g., human), such as by selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen (e.g., CD25, CTLA-4, PD-L1, PD-L2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating an infection (e.g., viral, fungal, bacterial, protozoal, or other parasitic infection) in an individual (e.g., human), such as by selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) a full-length antibody specifically recognizing a target antigen (e.g., CD25, CTLA-4, PD-L1, PD-L2, PD-1, CD3, CD4, CD123, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, there is provided a method of treating an infection (e.g., viral, fungal, bacterial, protozoal, or other parasitic infection) in an individual (e.g., human), such as by selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen on an immune cell (e.g., any immune cell surface antigen such as CD3, CD4, CD8, CD123, or CD25, or any immune checkpoint molecule expressed on immune cells such as PD-L1, PD-1, or CTLA-4); and b) a cytokine (e.g., any cytokine described herein, such as IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating an infection (e.g., viral, fungal, bacterial, protozoal, or other parasitic infection) in an individual (e.g., human), such as selectively directing cytokine activity to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen on an immune cell (e.g., any immune cell surface antigen such as CD3, CD4, CD8, CD123, or CD25, or any immune checkpoint molecule expressed on immune cells such as PD-L1, PD-1, or CTLA-4); and b) a cytokine (e.g., any cytokine described herein, such as IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, the immunocytokine or pharmaceutical composition thereof is administered intravenously or subcutaneously. In some embodiments, the immunocytokine or pharmaceutical composition thereof is administered in an amount of about 1 pg/kg to about 10 mg/kg. In some embodiments, the immunocytokine or pharmaceutical composition thereof is administered once every three weeks. In some embodiments, the method of treating infection described herein prevent worsening of, arrest and/or ameliorate at least one symptom of a pathogen infection in an individual in need thereof, reduce or eliminate pathogen, prevent damage to said individual or an organ or tissue of said individual, and/or prevent death. In some embodiments, the methods described herein can achieve one or more of the following: (a) controlling, ameliorating, and/or preventing tissue and/or organ injury or failure, such as induced by virus infection; (b) controlling, reducing, and/or inhibiting cell necrosis (such as reducing at least about 10% (including for example at least about any of 20%, 30%, 40%, 60%, 70%, 80%, 90%, or 100%) cell necrosis), such as necrosis in infected and/or non-infected tissue and/or organ; (c) controlling, and/or increasing the infiltration of inflammatory cells (e.g., NK cells, cytotoxic T cells, neutrophils) in infected tissues and/or organs, such as increasing at least about 10% (including for example at least about any of 20%, 30%, 40%, 60%, 70%, 80%, 90%, or 100%) inflammatory cell infiltration; (d) controlling, ameliorating and/or preventing inflammation in non-infected tissue and/or organ, systemic inflammation, and/or cytokine storm, such as downregulating at least about 10% (including for example at least about any of 20%, 30%, 40%, 60%, 70%, 80%, 90%, or 100%); (e) reducing mortality rate associated with pathogen infection, and/or preventing death, such as reducing at least about 10% (including for example at least about any of 20%, 30%, 40%, 60%, 70%, 80%, 90%, or 100%) death rate; and (f) reducing or eliminating at least about 10% (including for example at least about any of 20%, 30%, 40%, 60%, 70%, 80%, 90%, or 100%) pathogen.

In some embodiments, the methods described herein are for treating an immune disease, such as an autoimmune disease, or an immune suppression.

In some embodiments, the methods described herein are for treating immune suppression. Immunosuppression is a reduction or entirely absent of the activation or efficacy of the immune system, resulting in immune system's inability to fight diseases, for example infectious diseases or cancer. Immunosuppression can either be the result of diseases, or be produced by pharmaceuticals or an infection, resulting in an increased susceptibility to secondary infections by pathogens such as bacteria and viruses. Many diseases are characterized by the development of progressive immunosuppression in the patient. The presence of an impaired immune response in patients with malignancies (e.g. leukemia, lymphoma, multiple myeloma) is well documented. Progressive immunosuppression has also been observed in certain chronic infection such as AIDS, sepsis, leprosy, cytomegalovirus infections, malaria, lupus, and the like.

Immunodeficiency is also a potential adverse effect of many therapeutic treatments (radiotherapy or chemotherapy for example). By means of example and not limitation, diseases and conditions associated with immunodeficiency or immunosuppression comprise: human immunodeficiency virus (HV) infection and acquired immune deficiency syndrome (AIDS), hypogammaglobulinemia, hematologic cancers such as leukaemia and lymphoma, lymphocytopenia (lymphopenia) of any origin, lupus erythematosus, cachexia, opioids abuse, mastocytosis, rheumatic fever, trypanosomiasis, and alcohol abuse. In some embodiments, immunosuppression is associated with immune checkpoint signaling (e.g., PD-1 or CTLA-4 signaling). In such non-deliberate immunosuppression situations, patients are usually treated with immunostimulants (e.g. cytokines) to boost immune system. However, due to the lack of specificity, such immunostimulants activate the immune system in general and may trigger an overactivation of the immune system.

Thus in some embodiments, there is provided a method of treating an immune suppression in an individual (e.g., human), such as by selectively directing cytokine activity (e.g., immunostimulatory or pro-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of any of the IL-2/anti-PD-L1 immunocytokine, IL-10/anti-PD-L1 immunocytokine, IL-12/anti-PD-L1 immunocytokine, IL-23/anti-PD-L1 immunocytokine, IFN-γ/anti-PD-L1 immunocytokine, IFN-α/anti-PD-L1 immunocytokine, IL-2/PD-L2-Fc immunocytokine, IL-10/PD-L2-Fc immunocytokine, IL-12/PD-L2-Fc immunocytokine, IL-23/PD-L2-Fc immunocytokine, IFN-γ/PD-L2-Fc immunocytokine, IFN-α/PD-L2-Fc immunocytokine, IL-2/anti-PD-1 immunocytokines, IL-12/anti-PD-1 immunocytokines, IL-23/anti-PD-1 immunocytokines, IFN-γ/anti-PD-1 immunocytokines, IFN-α/anti-PD-1 immunocytokines, IL-2/anti-CTLA-4 immunocytokines, IL-12/anti-CTLA-4 immunocytokines, IL-23/anti-CTLA-4 immunocytokines, IFN-γ/anti-CTLA-4 immunocytokines, IFN-α/anti-CTLA-4 immunocytokines, IL-2/anti-CD3 immunocytokines, IL-12/anti-CD3 immunocytokines, IL-23/anti-CD3 immunocytokines, IFN-γ/anti-CD3 immunocytokines, IFN-α/anti-CD3 immunocytokines, IL-2/anti-CD4 immunocytokines, IL-12/anti-CD4 immunocytokines, IL-23/anti-CD4 immunocytokines, IFN-γ/anti-CD4 immunocytokines, IFN-α/anti-CD4 immunocytokines, IL-2/anti-CD8 immunocytokines, IL-12/anti-CD8 immunocytokines, IL-23/anti-CD8 immunocytokines, IFN-γ/anti-CD8 immunocytokines, IFN-α/anti-CD8 immunocytokines, IL-2/anti-CD25 immunocytokines, IL-12/anti-CD25 immunocytokines, IL-23/anti-CD25 immunocytokines, IFN-γ/anti-CD25 immunocytokines, and IFN-α/anti-CD25 immunocytokines described herein, or pharmaceutical composition thereof. In some embodiments, there is provided a method of treating an immune suppression in an individual (e.g., human), such as by selectively directing cytokine activity (e.g., immunostimulatory or pro-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: ) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., PD-1, PD-L1, CTLA-4, CD3, CD4, CD8, CD123, or CD25) on an immune cell; and b) an immunostimulatory cytokine (e.g., IL-2, IFN-α (e.g., IFN-α2b), IFN-γ, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating an immune suppression in an individual (e.g., human), such as by selectively directing cytokine activity (e.g., immunostimulatory or pro-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen (e.g., PD-1, PD-L1, CTLA-4, CD3, CD4, CD8, CD123, or CD25) on an immune cell; and b) an immunostimulatory cytokine (e.g., IL-2, IFN-α (e.g., IFN-α2b), IFN-γ, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating an immune suppression in an individual (e.g., human), such as by selectively directing cytokine activity (e.g., immunostimulatory or pro-inflammatory cytokine activity) to an immune cell (e.g., T cell, B cell, monocyte, NK cell, myeloid DC cell, plasma DC cell, macrophage), comprising administering to the individual an effective amount of an immunocytokine (or pharmaceutical composition thereof) comprising: a) a full-length antibody specifically recognizing a target antigen (e.g., PD-1, PD-L1, CTLA-4, CD3, CD4, CD8, CD123, or CD25) on an immune cell; and b) an immunostimulatory cytokine (e.g., IL-2, IFN-α (e.g., IFN-α2b), IFN-γ, IL-12, or IL-23) or variant thereof, wherein the cytokine or variant thereof is positioned at the hinge region (e.g., between the C-terminus of CH1 and the N-terminus of the hinge region) of a heavy chain of the full-length antibody. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the methods of treating an immune suppression described herein activate or enhance immune response, increase CD8 to CD4 ratio, promote immune cell proliferation and/or differentiation, induce or enhance cytokine release (e.g., IL-2, IL-6, IFN-γ), prevent worsening of, arrest and/or ameliorate at least one symptom of an immune suppression in an individual in need thereof, and/or prevent death.

In some embodiments, the methods described herein are for treating autoimmune diseases. Autoimmune disease is a disease resulting from an immune response against a self-tissue or tissue component, including both self-antibody responses and cell-mediated responses. The term “autoimmune disease,” as used herein, encompasses organ-specific autoimmune diseases, in which an autoimmune response is directed against a single tissue, such as type I diabetes mellitus (T1D), Crohn's disease, ulcerative colitis, myasthenia gravis, vitiligo, Graves' disease, Hashimoto's disease, Addison's disease and autoimmune gastritis and autoimmune hepatitis. The term “autoimmune disease” also encompasses non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include pernicious anemia including some of autoimmune gastritis, primary biliary cirrhosis, autoimmune thrombocytopenia, Sjogren's syndrome, multiple sclerosis and psoriasis. In some embodiments, the autoimmune disease is selected from the group consisting of diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, inflammatory bowel disease (IBD), Crohn's disease, Graves ophthalmopathy, sarcoidosis, primary biliary cirrhosis, uveitis posterior, and interstitial lung fibrosis. One skilled in the art understands that the methods of the invention can be applied to these or other autoimmune diseases, as desired.

Thus in some embodiments, there is provided a method of treating an autoimmune disease in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of any of the IL-10/anti-CD4 immunocytokines, IL-10/anti-CD3 immunocytokines, IL-10/anti-CD8 immunocytokines, IL-10/anti-CD25 immunocytokines, IL-10/anti-PD-1 immunocytokines, IL-10/anti-PD-L1 immunocytokines, IL-10/anti-CTLA-4 immunocytokines, and IL-10/PD-L2-Fc immunocytokines described herein, or pharmaceutical composition thereof. In some embodiments, there is provided a method of treating an autoimmune disease in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., PD-1, PD-L1, CTLA-4, CD3, CD4, CD8, or CD25) on an immune cell; and b) an immunosuppressive cytokine (e.g., IL-10, IL-27, IL-35, or TGF-β) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating an autoimmune disease in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen (e.g., PD-1, CTLA-4, PD-L1, CD3, CD4, CD123, CD25, or CD8) on an immune cell; and b) an immunosuppressive cytokine (e.g., IL-10, IL-27, IL-35, or TGF-β) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating an autoimmune disease in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) a full-length antibody specifically recognizing a target antigen (e.g., CTLA-4, PD-L1, PD-1, CD3, CD4, CD123, CD25, or CD8) on an immune cell; and b) an immunosuppressive cytokine (e.g., IL-10, IL-27, IL-35, or TGF-β) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the methods of treating an autoimmune disease described herein prevent worsening of, arrest and/or ameliorate at least one symptom of an autoimmune disease in an individual in need thereof, prevent damage to healthy self tissues or organ, control, ameliorate and/or prevent infiltration of immune cells to healthy self tissue and/or organ, systemic inflammation, and/or cytokine storm, and/or prevent death.

In some embodiments, there is provided a method of treating a graft rejection or GvHD in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of any of the IL-10/anti-CD4 immunocytokines, IL-10/anti-CD3 immunocytokines, IL-10/anti-CD8 immunocytokines, IL-10/anti-CD25 immunocytokines, IL-10/anti-PD-1 immunocytokines, IL-10/anti-PD-L1 immunocytokines, IL-10/anti-CTLA-4 immunocytokines, and IL-10/PD-L2-Fc immunocytokines described herein, or pharmaceutical composition thereof. In some embodiments, there is provided a method of treating a graft rejection or GvHD in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., PD-1, PD-L1, PD-L2, CTLA-4, CD3, CD4, CD8, CD123, or CD25) on an immune cell; and b) an immunosuppressive cytokine (e.g., IL-10, IL-27, IL-35, or TGF-β) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), and wherein the cytokine or variant thereof is positioned at (e.g., at the N′ of, at the C′ of, or within) the hinge region. In some embodiments, there is provided a method of treating a graft rejection or GvHD in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) an antibody (e.g., full-length antibody, heavy chain only antibody, or antigen-binding fragment fused to an Fc domain subunit or portion thereof via a hinge region) specifically recognizing a target antigen (e.g., PD-1, PD-L1, PD-L2, CTLA-4, CD3, CD4, CD25, CD123, or CD8) on an immune cell; and b) an immunosuppressive cytokine (e.g., IL-10, IL-27, IL-35, or TGF-β) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain. In some embodiments, there is provided a method of treating a graft rejection or GvHD in an individual (e.g., human), such as by selectively directing cytokine activity (e.g., immunosuppressive or anti-inflammatory cytokine activity) to an immune cell (e.g., T cell, B cell, monocyte, NK cell, myeloid DC cell, plasma DC cell, macrophage) in an individual (e.g., human), comprising administering to the individual an effective amount of an immunocytokine (or pharmaceutical composition thereof) comprising: a) a full-length antibody specifically recognizing a target antigen (e.g., CTLA-4, PD-L1, PD-L2, PD-1, CD3, CD4, CD25, CD123, or CD8) on an immune cell; and b) an immunosuppressive cytokine (e.g., IL-10, IL-27, IL-35, or TGF-β) or variant thereof, wherein the cytokine or variant thereof is positioned at the hinge region (e.g., within the hinge region, or between the C-terminus of CH1 and the N-terminus of the hinge region) of a heavy chain of the full-length antibody. In some embodiments, the immunocytokine or pharmaceutical composition thereof is administered intravenously, subcutaneously, or intratumorally. In some embodiments, the immunocytokine or pharmaceutical composition thereof is administered in an amount of about 1 μg/kg to about 10 mg/kg. In some embodiments, the immunocytokine or pharmaceutical composition thereof is administered once every three weeks. In some embodiments, the methods of treating a graft rejection described herein prevent worsening of, arrest and/or ameliorate at least one symptom of a graft rejection in an individual in need thereof; prevent damage to donor/foreign tissues or organ; control, ameliorate and/or prevent infiltration of immune cells to donor/foreign tissues or organ, systemic inflammation, and/or cytokine storm; reduce Th17 cell activation; improve graft survival; prolong survival, increase survival rate, and/or prevent death. In some embodiments, the methods of treating a GvHD described herein prevent worsening of, arrest and/or ameliorate at least one symptom of a GvHD in an individual in need thereof; reduce Th17 cell activation; prevent damage to self/healthy tissues or organ; control, ameliorate and/or prevent infiltration of immune cells to self/healthy tissues or organ, systemic inflammation, and/or cytokine storm; improve graft survival; prolong survival, increase survival rate, and/or prevent death; and/or improve disease activity score (see, e.g., P.J. Martin, Biol Blood Marrow Transplant. 2009 July; 15(7):777-784).

In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD123, CD25, HER2, PD-1, CD3, CD4, or CD8) in an individual (e.g., human), comprising administering to the individual an effective amount of an immunocytokine (or pharmaceutical compositions thereof), wherein the immunocytokine comprises: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD123, CD25, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the cytokine or variant thereof is positioned at (e.g., at the N′ of, at the C′ of, or within) the hinge region; and wherein the activity of the cytokine or variant thereof is selectively activated upon binding of the antigen-binding protein to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunocytokine (or pharmaceutical compositions thereof), wherein the immunocytokine comprises: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain; and wherein the activity of the cytokine or variant thereof is selectively activated upon binding of the antibody to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunocytokine (or pharmaceutical compositions thereof), wherein the immunocytokine comprises: a) an antibody (e.g., full-length antibody, or antigen-binding fragment fused to an Fc domain subunit or portion thereof via a hinge region) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising from N-terminus to C-terminus: a VH domain, optionally a CH1 domain, the cytokine or variant thereof at a hinge region, a CH2 domain, and optionally a CH3 domain; and wherein the activity of the cytokine or variant thereof is selectively activated upon binding of the antibody to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunocytokine (or pharmaceutical compositions thereof), wherein the immunocytokine comprises: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; and wherein the activity of the cytokine or variant thereof is selectively activated upon binding of the full-length antibody to the target antigen. In some embodiments, in the presence of binding of the antigen-binding protein (e.g., antibody such as full-length antibody, or antigen-binding fragment-hinge-Fc fusion protein such as ligand/receptor-hinge-Fc fusion protein) or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or antigen-binding fragment-hinge-Fc fusion protein such as ligand/receptor-hinge-Fc fusion protein) or antigen-binding fragment (e.g., ligand, receptor, VHH, scFv, Fab) to the target antigen. In some embodiments, in the absence of binding of the antigen-binding protein (e.g., antibody such as full-length antibody, or antigen-binding fragment-hinge-Fc fusion protein such as ligand/receptor-hinge-Fc fusion protein or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%,, i⁰, 0.9%, 0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state.

Administration of the immunocytokines described herein or pharmaceutical compositions thereof may be carried out in any convenient manner, including by injection or transfusion. The route of administration is in accordance with known and accepted methods, such as by single or multiple bolus or infusion over a long period of time in a suitable manner. The immunocytokines or pharmaceutical compositions thereof may be administered to a patient transarterially, subcutaneously, intradermally, intratumorally, intranodally, intramedullary, intramuscularly, intravenously, or intraperitoneally. In some embodiments, the immunocytokine or pharmaceutical composition thereof is administered systemically. In some embodiments, the immunocytokine or pharmaceutical composition thereof is administered to an individual by infusion, such as intravenous infusion. Infusion techniques for immunotherapy are known in the art (see, e.g., Rosenberg et al., New Eng. J. of Med. 319: 1676 (1988)). In some embodiments, the immunocytokine or pharmaceutical composition thereof is administered to an individual by intradermal or subcutaneous (i.e., beneath the skin) injection. For subcutaneous injections, the immunocytokines or pharmaceutical compositions may be injected using a syringe. However, other devices for administration of the immunocytokines or pharmaceutical compositions are available such as injection devices; injector pens; auto-injector devices, needleless devices; and subcutaneous patch delivery systems. In some embodiments, the immunocytokine or pharmaceutical composition thereof is administered by intravenous injection. In some embodiments, the immunocytokine or pharmaceutical composition thereof is injected directly into a tumor, or a lymph node. In some embodiments, the immunocytokine or pharmaceutical composition thereof is administered locally to a site of tumor, such as directly into tumor cells, or to a tissue having tumor cells. In some embodiments, the immunocytokine or pharmaceutical composition thereof is administered by sustained release or extended-release means.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics andNew Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

When in vivo administration of the immunocytokines described herein or pharmaceutical compositions thereof are used, normal dosage amounts may vary from about 1 μg/kg to about 10 mg/kg of mammal body weight depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays. In some embodiments, the immunocytokine described herein or pharmaceutical composition thereof is administered in an amount of about 1 μg/kg to about 10 mg/kg, such as any of about 1 μg/kg to about 500 μg/kg, about 500 μg/kg to about 1 mg/kg, about 1 mg/kg to about 10 mg/kg, about 1 μg/kg to about 1 mg/kg, about 1 μg/kg to about 200 μg/kg, about 100 μg/kg to about 500 μg/kg, about 100 μg/kg to about 1 mg/kg, or about 500 μg/kg to about 1 mg/kg.

In some embodiments, the immunocytokine described herein or pharmaceutical composition thereof is administered (e.g., infused) to the individual (e.g., human) over a period of time no more than about any of 24 hours, 20 hours, 15 hours, 10 hours, 8 hours, 6 hours, 3 hours, 2 hours, 1 hours, 30 minutes, or less. In some embodiments, the immunocytokine described herein or pharmaceutical composition thereof is administered (e.g., infused) to the individual (e.g., human) over a period of time of any one of about 30 minutes to about 1 hour, about 1 hour to about 2 hours, about 2 hours to about 4 hours, about 4 hours to about 6 hours, about 6 hours to about 8 hours, about 8 hours to about 10 hours, about 10 hours to about 12 hours, about 12 hours to about 18 hours, about 18 hours to about 24 hours, about 30 minutes to about 2 hours, about 2 hours to about 5 hours, about 5 hours to about 10 hours, about 10 hours to about 20 hours, about 30 minutes to about 10 hours, or about 30 minutes to about 24 hours.

In some embodiments, the immunocytokine described herein or pharmaceutical composition thereof is administered for a single time (e.g., bolus injection). In some embodiments, the immunocytokine described herein or pharmaceutical composition thereof is administered for multiple times (such as any of 2, 3, 4, 5, 6, or more times). If multiple administrations, they may be performed by the same or different routes and may take place at the same site or at alternative sites. The immunocytokine described herein or pharmaceutical composition thereof may be administered daily to once per year. The interval between administrations can be about any one of 24 hours to a year. Intervals can also be irregular (e.g., following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly. In some embodiments, the immunocytokine described herein or pharmaceutical composition thereof is administered once per day (daily), once per 2 days, once per 3 days, once per 4 days, once per 5 days, once per 6 days, once per week, once per 10 days, once every 2 weeks, once every 3 weeks, once every 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. In some embodiments, the immunocytokine described herein or pharmaceutical composition thereof is administered once every three weeks.

In some embodiments, the pharmaceutical composition is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### VII. Articles of Manufacture and Kits

Further provided are kits, unit dosages, and articles of manufacture comprising any of the immunocytokines described herein. In some embodiments, a kit is provided which contains any one of the pharmaceutical compositions described herein and preferably provides instructions for its use, such as for use in the treatment of the disorders described herein (e.g., cancer, infection, or autoimmune disease).

Kits of the invention include one or more containers comprising an immunocytokine described herein for treating a disease. For example, the instructions comprise a description of administration of the immunocytokine to treat a disease, such as cancer. The kit may further comprise a description of selecting an individual (e.g., human) suitable for treatment based on identifying whether that individual has the disease and the stage of the disease. The instructions relating to the use of the immunocytokine generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is an immunocytokine as described herein. The container may further comprise a second pharmaceutically active agent. The kits may optionally provide additional components such as buffers and interpretive information. Normally, the kit comprises a container and a label or package insert(s) on or associated with the container.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXAMPLES

The examples below are intended to be purely exemplary of the invention and should therefore not be considered to limit the invention in any way. The following examples and detailed description are offered by way of illustration and not by way of limitation.

### Example 1: Position of Cytokine or Variant Thereof within the Immunocytokine Affects Biological Activity of the Cytokine or Variant Thereof

### Construction of IL-2/Anti-HER2 Immunocytokines of Various Formats

Anti-HER2 antibody comprising trastuzumab (Herceptin®) VH (SEQ ID NO: 150) and VL (SEQ ID NO: 151) sequences was used as the parental full-length antibody (FIG. 1A), comprising two light chains each comprising the amino acid sequence of SEQ ID NO: 154. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. An IL-2 variant comprising R38D/K43E/E61R triple mutations (SEQ ID NO: 2) was used as the cytokine variant, which block the binding of IL-2 to IL-2Rα (CD25). The IL-2 variant was positioned at below positions for constructing various IL-2/anti-HER2 immunocytokines: i) an IL-2 variant was fused to the N-terminus of a subunit of the Fc fragment (see FIG. 1B for exemplary structure), hereinafter referred to as “IL-2 mutant-Fc-Her2 Ab”; ii) an IL-2 variant was fused to the C-terminus of a heavy chain of the anti-HER2 antibody (see FIG. 1C for exemplary structure), hereinafter referred to as “Fab-Fc-IL-2 mutant-Her2 Ab”; and iii) an IL-2 variant was positioned within the hinge region of a heavy chain of the anti-HER2 antibody (see FIG. 2A for exemplary structure), hereinafter referred to as “Fab-IL-2 mutant-Fc-Her2 Ab.” The Fab-IL-2 mutant-Fc-Her2 Ab immunocytokine comprises two light chains each comprising the amino acid sequence of SEQ ID NO: 154, one heavy chain comprising the amino acid sequence of SEQ ID NO: 155, and one heavy chain with the IL-2 variant positioned at the hinge region comprising the amino acid sequence of SEQ ID NO: 156.

Nucleic acids encoding various formats of IL-2/anti-HER2 immunocytokines were chemically synthesized, cloned into a lentiviral vector, and transfected into CHO cells for immunocytokine expression. Expressed immunocytokines were collected from supernatant, purified by protein A chromatography, and verified on SDS-PAGE for purity.

### IL-2 Signal Transduction Assay

HEK-Blue™ IL-2 Cells (InvivoGen Cat. #hkb-i12) and HEK-PD-1-IL-2 cells (generated in-house by overexpressing human PD-I in HEK-Blue™ IL-2 Cells using a lentiviral vector) were used to assess IL-2 signal activation activity of the various IL-2/anti-HER2 immunocytokines, following the InvivoGen user manual (InvivoGen Cat. #hkb-i12), hereinafter also referred to as “HEK-IL-2 reporter assay” or “HEK-PD-1-IL-2 reporter assay.” HEK-Blue™ IL-2 reporter cells and HEK-PD-1-IL-2 reporter cells stably express the human IL-2 receptor (human IL-2Rα, IL-2RD, and IL-2Rγ), along with the human JAK3 and STAT5 genes to obtain a fully functional IL-2 signaling pathway. In addition, these reporter cells also carry a STAT5-inducible secreted embryonic alkaline phosphatase (SEAP) reporter gene. Upon IL-2 stimulation, HEK-Blue™ IL-2 reporter cells and HEK-PD-1-IL-2 reporter cells trigger the activation of STAT5 and the subsequent secretion of SEAP, the levels of which can be monitored using QUANTI-Blue™ (InvivoGen Cat. #rep-qbs) colorimetric enzyme assay for alkaline phosphatase activity.

Briefly, HEK-Blue™ IL-2 cells were added to various IL-2/anti-HER2 immunocytokines in each plate well (or recombinant human IL-2 in a well as positive control, anti-HER2 antibody trastuzumab (Herceptin®) in a well as negative control), and incubated at 37° C. in a C02 incubator for 20-24 hours or overnight. After incubation, supernatant was transferred to fresh plate wells, added QUANTI-Blue™ solution, and incubated at 37° C. incubator for 30 minutes-3 hours. Then SEAP levels were determined using a spectrophotometer at 620-655 nm. The activity of recombinant human IL-2 (positive control) in activating IL-2 signaling pathway was measured as 10 unit/ng and served as a reference. Percent IL-2 signal transduction for various IL-2/anti-HER2 immunocytokines was calculated by dividing the IL-2/anti-HER2 immunocytokine readout by the recombinant human IL-2 readout.

### PBMC Proliferation Assay

Biological activity of IL-2 can also be tested by peripheral blood mononuclear cell (PBMC) proliferation/survival assay. IL-2 is essential for the proliferation and survival of activated T-cells. Human PBMCs (80,000 cells/well) were stimulated by an anti-CD3 antibody (OKT3, 0.5 pg/mL) in the presence of increasing concentrations of recombinant human IL-2 (“rIL-2”; 0, 0.04, 0.2, 1.0, or 5.0 ng/mL). 1 ng/mL of rIL-2 was determined to be the minimal concentration required for T-cell proliferation, based on PBMC cell number (≤80,000 cells/well) and viability after a 6-day culture. To determine the minimal concentration of IL-2/anti-HER2 immunocytokine required for T-cell proliferation, PBMCs (80,000 cells/well) were stimulated by an anti-CD3 antibody (OKT3, 0.5 μg/mL) in the presence of increasing concentrations of various formats of IL-2/anti-HER2 immunocytokines (0, 0.32, 1.6, 8, 40, 200, or 1000 ng/mL). Free state rIL-2 served as positive control (0.2 or 1 ng/mL). Percent T-cell proliferation of IL-2/anti-HER2 immunocytokine relative to rIL-2 was calculated by normalizing to corresponding molecular weights. The molecular weights of IL-2/anti-HER2 immunocytokine and rIL-2 are about 162 kDa and 12 kDa, respectively, hence about 13 ng of IL-2/anti-HER2 immunocytokine is equivalent to about 1 ng of rIL-2 for the same IL-2 molar concentration.

As can be seen from Table 1, when the IL-2 variant was fused to the N-terminus of a subunit of the Fc fragment of the anti-HER2 antibody (IL-2 mutant-Fc-Her2 Ab), such IL-2 variant retained about 12.0% biological activity as measured by IL-2 signal transduction assay, and about 4.3% biological activity as measured by PBMC proliferation assay. When the IL-2 variant was fused to the C-terminus of a heavy chain of the anti-HER2 antibody (Fab-Fc-IL-2 mutant-Her2 Ab), such IL-2 variant retained about 6.3% biological activity as measured by IL-2 signal transduction assay, and about 3.1% biological activity as measured by PBMC proliferation assay.

When the IL-2 variant was positioned at the hinge region of a heavy chain of the anti-HER2 antibody (Fab-IL-2 mutant-Fc-Her2 Ab), such IL-2 variant only retained about 2.3% biological activity as measured by IL-2 signal transduction assay, and lost all biological activity as measured by PBMC proliferation assay. These results can be considered as reflecting IL-2 variant activity in the absence of binding of the anti-HER2 antigen-binding fragment to HER2, since HEK-IL-2 cells and PBMCs have minimal HER2 expression on the cell surface.

Hence, cytokine (e.g., IL-2 variant) positioned at the hinge region of a heavy chain of a full-length antibody (e.g., anti-HER2 antibody) in the absence of binding of the antibody to the target antigen (e.g., HER2) showed more restricted biological activity compared to when such cytokine was positioned at the N-terminus of a subunit of the Fc fragment, or at the C-terminus of a heavy chain of the full-length antibody, such as measured by signal transduction assay or PBMC proliferation assay.

TABLE 1
IL-2 biological activity of various
IL-2/anti-HER2 immunocytokine formats

| | Percent IL-2 | Percent T-cell |
|---|---|---|
| | signal transduction | proliferation |
| | (HEK-IL-2 | (PBMC proliferation |
| | reporter assay) | assay) |
| Recombinant human IL-2 | 100.0% | 100.0% |
| (free state) | | |
| Anti-HER2 antibody | 0.0% | 0.0% |
| (trastuzumab Herceptin ®) | | |
| IL-2 mutant-Fc-HER2 Ab | 12.0% | 4.3% |
| Fab-Fc-IL-2 mutant-HER2 | 6.3% | 3.1% |
| Ab | | |
| Fab-IL-2 mutant-Fc-HER2 | 2.3% | 0.0% |
| Ab | | |

### Example 2: Immunocytokine with Cytokine Positioned at the Hinge Region Demonstrates Target Antigen Binding-Dependent Cytokine Activity

An anti-CD3 antibody specifically recognizing CD3E (made in-house) comprising VH (SEQ ID NO: 91) and VL (SEQ ID NO: 92) sequences was used as the parental full-length antibody, comprising two light chains each comprising the amino acid sequence of SEQ ID NO: 94. This anti-CD3E antibody does not compete with OKT3 antibody for binding to CD3, and is unable to activate TCR signaling (i.e., non-active anti-CD3 antibody), thus cannot stimulate PBMC or T cell proliferation. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. The IL-2 variant comprising R38D/K43E/E61R triple mutations (SEQ ID NO: 2) was positioned at below positions for constructing various IL-2/anti-CD3 immunocytokines: i) an IL-2 variant was fused to the N-terminus of a subunit of the Fc fragment (see FIG. 1B for exemplary structure), hereinafter referred to as “IL-2 mutant-Fc-CD3 Ab”; ii) an IL-2 variant was fused to the C-terminus of a heavy chain of the anti-CD3 antibody (see FIG. 1C for exemplary structure), hereinafter referred to as “Fab-Fc-IL-2 mutant-CD3 Ab”; and iii) an IL-2 variant was positioned within the hinge region of a heavy chain of the anti-CD3 antibody (see FIG. 2A for exemplary structure), hereinafter referred to as “Fab-IL-2 mutant-Fc-CD3 Ab.” The Fab-IL-2 mutant-Fc-CD3 Ab immunocytokine comprises two light chains each comprising the amino acid sequence of SEQ ID NO: 94, one heavy chain comprising the amino acid sequence of SEQ ID NO: 95, and one heavy chain with the IL-2 variant positioned at the hinge region comprising the amino acid sequence of SEQ ID NO: 96. IL-2/anti-CD3 immunocytokines were constructed, expressed, and purified as described in Example 1. The binding activity of different formats of IL-2/anti-CD3 immunocytokines to CD3+ T cells was verified using FACS.

IL-2 signal transduction assay using HEK-Blue™ IL-2 cells, and PBMC proliferation assay were similarly conducted as described in Example 1, with various formats of IL-2/anti-CD3 immunocytokines, rIL-2 (positive control), or anti-CD3 antibody (made in-house; negative control).

TABLE 2
IL-2 biological activity of various
IL-2/anti-CD3 immunocytokine formats

| | Percent IL-2 | Percent T-cell |
|---|---|---|
| | signal transduction | proliferation |
| | (HEK-IL-2 | (PBMC proliferation |
| | reporter assay) | assay) |
| Recombinant human IL-2 | 100.0% | 100.0% |
| (free state) | | |
| Anti-CD3 antibody (in- | 0.0% | 0.0% |
| house) | | |
| IL-2 mutant-Fc-CD3 Ab | 11.0% | 39.0% |
| Fab-Fc-IL-2 mutant-CD3 Ab | 6.7% | 52.0% |
| Fab-IL-2 mutant-Fc-CD3 Ab | 2.5% | 45.0% |

Since HEK-IL-2 cells have no CD3 expression on the cell surface, IL-2 signal transduction assay with HEK-IL-2 reporter cells reflects IL-2 variant activity in the absence of binding of the anti-CD3 antigen-binding fragment to CD3. As can be seen from Table 2, when the IL-2 variant was fused to the N-terminus of a subunit of the Fc fragment of the anti-CD3 antibody (IL-2 mutant-Fc-CD3 Ab), such IL-2 variant retained about 11.0% biological activity; when the IL-2 variant was fused to the C-terminus of a heavy chain of the anti-CD3 antibody (Fab-Fc-IL-2 mutant-CD3 Ab), such IL-2 variant retained about 6.7% biological activity; when the IL-2 variant was positioned at the hinge region of a heavy chain of the anti-CD3 antibody (Fab-IL-2 mutant-Fc-CD3 Ab), such IL-2 variant only retained about 2.5% biological activity. This result is consistent with that shown in Table 1 for IL-2/anti-HER2 immunocytokines as measured by IL-2 signal transduction assay.

In the presence of binding of the anti-CD3 antigen-binding fragment to CD3 in PBMC (CD3+ T cells), IL-2/anti-CD3 immunocytokines all demonstrated much higher IL-2 biological activity compared to that without CD3 binding. As shown in PBMC proliferation assay in Table 2, when the IL-2 variant was fused to the N-terminus of a subunit of the Fc fragment of the anti-CD3 antibody (IL-2 mutant-Fc-CD3 Ab), the biological activity of the IL-2 variant increased to about 39.0% upon anti-CD3 antibody/CD3 binding; when the IL-2 variant was fused to the C-terminus of a heavy chain of the anti-CD3 antibody (Fab-Fc-IL-2 mutant-CD3 Ab), the biological activity of the IL-2 variant increased to about 52.0% upon anti-CD3 antibody/CD3 binding. Such increase in cytokine biological activity was likely because antigen-antibody binding brought the cytokine moiety close to its receptor and increased the likelihood of cytokine-receptor binding, and/or due to the stabilization effect from antibody-antigen binding. Surprisingly, IL-2 variant positioned at the hinge region of a heavy chain of the anti-CD3 antibody revealed IL-2 biological activity upon anti-CD3 antibody/CD3 binding, increased to about 45.0%, which was even stronger than that of cytokine N-terminus fusion (IL-2 mutant-Fc-CD3 Ab).

In view of results from Tables 1 and 2, the biological activity of cytokine (e.g., IL-2 variant) positioned at the hinge region of a heavy chain of the full-length antibody is target antigen (e.g., CD3) binding-dependent. IL-2/anti-HER2 immunocytokines, regardless of format, were unable to bind to T cells or HEK-IL-2 cells. Especially for immunocytokine with cytokine positioned at the hinge region of a heavy chain of the full-length antibody, without target antigen-binding by the antibody portion, the biological activity of such cytokine cannot be revealed. See 0.0% PBMC proliferation in Table 1 for Fab-IL-2 mutant-Fc-Her2 Ab. On the contrary, IL-2/anti-CD3 immunocytokines, which are able to bind to T cells via CD3, revealed the biological activity of cytokine positioned at the hinge region of a heavy chain of the full-length antibody. See 45.0% PBMC proliferation in Table 2 for Fab-IL-2 mutant-Fc-CD3 Ab. These results suggest that immunocytokine comprising cytokine positioned at the hinge region of a heavy chain of a full-length antibody may provide target specificity and reduce cytokine storm.

### Example 3: Immunocytokines with Cytokine Positioned at the Hinge Region Favor Target Antigen-Antibody (or Ligand-Receptor) Binding First, then Cytokine-Cytokine Receptor Binding Second

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. The IL-2 variant comprising R38D/K43E/E61R triple mutations (SEQ ID NO: 2) was positioned within the hinge region of a heavy chain of the anti-PD-1 antibody (see FIG. 2A for exemplary structure), to construct the IL-2/anti-PD-1 immunocytokine “Fab-IL-2 mutant-Fc-PD-1 Ab.” The Fab-IL-2 mutant-Fc-PD-1 Ab immunocytokine (or “IL-2(R38D/K43E/E61R)/anti-PD-1 immunocytokine”) comprises two light chains each comprising the amino acid sequence of SEQ ID NO: 106, one heavy chain comprising the amino acid sequence of SEQ ID NO: 107, and one heavy chain with the IL-2 variant positioned at the hinge region comprising the amino acid sequence of SEQ ID NO: 108.

A PD-L2-Fc fusion protein (PD-L2 extracellular domain fused to the N-terminus of an Fc fragment through an N′ truncated IgG1 hinge (SEQ ID NO: 50); SEQ ID NO: 177) was used as another parental construct to construct immunocytokines that bind to PD-1. To construct heterodimeric PD-L2-Fc fusion protein, one PD-L2-Fc fusion polypeptide comprises a hinge region comprising SEQ ID NO: 52, and an Fc domain subunit comprising SEQ ID NO: 61; the other PD-L2-Fc fusion polypeptide comprises a hinge region comprising SEQ ID NO: 51, and an Fc domain subunit comprising SEQ ID NO: 62. The IL-2/PD-L2-Fc immunocytokine “ligand-IL-2 mutant-PD-L2-Fc” or “IL-2(R38D/K43E/E61R)/PD-L2-Fc immunocytokine” (or “construct #11”) comprises one fusion polypeptide (SEQ ID NO: 180) from N′ to C′: PD-L2 extracellular domain—GGGGS linker (SEQ ID NO: 213)—IL-2 variant (SEQ ID NO: 2)—N′ truncated IgG1 hinge (SEQ ID NO: 52)—an Fc fragment, and one pairing polypeptide (SEQ ID NO: 179) comprising from N′ to C′: PD-L2 extracellular domain—GGGGS linker (SEQ ID NO: 213)—IL-2 variant (SEQ ID NO: 2)—N′ truncated IgG1 hinge (SEQ ID NO: 51)—a pairing Fc fragment.

Fab-IL-2 mutant-Fc-PD-1 Ab and ligand-IL-2 mutant-PD-L2-Fc were constructed, expressed, and purified as described in Example 1. FACS was used to confirm that both Fab-IL-2 mutant-Fc-PD-1 Ab and ligand-IL-2 mutant-PD-L2-Fc bind to HEK-PD-I-IL-2 cells (generated in-house by overexpressing human PD-1 in HEK-Blue™ IL-2 Cells using a lentiviral vector), but not to HEK-Blue™ IL-2 Cells (InvivoGen Cat. #hkb-i12).

HEK-Blue™ IL-2 Cells and HEK-PD-1-IL-2 cells were used to assess IL-2 signal activation activity of Fab-IL-2 mutant-Fc-PD-1 Ab and ligand-IL-2 mutant-PD-L2-Fc, as described in Example 1. Recombinant human IL-2 (rIL-2) served as positive control. Anti-PD-1 antibody nivolumab (Opdivo®) and parental PD-L2-Fc fusion protein (each polypeptide chain comprises SEQ ID NO: 177) served as negative controls.

TABLE 3
IL-2 biological activity of Fab-IL-2 mutant-
Fc-PD-1 Ab and ligand-IL-2 mutant-PD-L2-Fc

| | Percent IL-2 | Percent IL-2 signal |
|---|---|---|
| | signal transduction | transduction |
| | (HEK-IL-2 | (HEK-PD-1-IL-2 |
| | reporter assay) | reporter assay) |
| Recombinant human IL-2 | 100.0% | 100.0% |
| (free state) | | |
| Anti-PD-1 antibody | 0.0% | 0.0% |
| (nivolumab (Opdivo ®)) | | |
| Parental PD-L2-Fc fusion | 0.0% | 0.0% |
| protein | | |
| Fab-IL-2 mutant-Fc-PD-1 Ab | 2.1% | 35.2% |
| ligand-IL-2 mutant-PD-L2-Fc | 2.4% | 43.5% |

Consistent with data shown in Examples 1 and 2, in the absence of target antigen (PD-1) binding, IL-2 positioned at the hinge region of the immunocytokine showed little biological activity (2.1% or 2.4%) compared to free state rIL-2 (100.0%), as measured by HEK-IL-2 reporter assay. Comparing HEK-IL-2 reporter assay and HEK-PD-I-IL-2 reporter assay results in Table 3, binding of anti-PD-1 antigen-binding fragment or PD-L2 extracellular domain to PD-1 on cell surface greatly facilitated the engagement of IL-2 variant with IL-2 receptor. In other words, immunocytokines with cytokine positioned at the hinge region favored target antigen-antibody binding (Fab-IL-2 mutant-Fc-PD-1 Ab, 35.2% vs. 2.1%) or ligand-receptor binding (ligand-IL-2 mutant-PD-L2-Fc, 43.5% vs. 2.4%) first, then cytokine-cytokine receptor binding second.

### Example 4: Generation of IL-12 Variants with Reduced Biological Activity in the Absence of Target Antigen-Binding of Immunocytokines

### Construction of IL-12 Variants and Immunocytokines Thereof

IL-12 is a heterodimeric cytokine composed of covalently linked p35 and p40 subunits. IL-12 variants comprising amino acid substitution in the p40 subunit were constructed by replacing amino acids from position 56 to 65 of the p40 subunit with Alanine or Serine (see Table 4), and a single chain IL-12 variant was made, from N′ to C′: p40 variant subunit—linker (SEQ ID NO: 228)—p35 wildtype subunit (SEQ ID NO: 29). A single chain “wildtype” IL-12 was also constructed as a control (SEQ ID NO: 35), from N′ to C′: p40 wildtype subunit-linker (SEQ ID NO: 228)—p35 wildtype subunit, referred to as “WT” in Table 4.

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Various single chain IL-12 variants (or single chain “wildtype” IL-12 control) were positioned within the hinge region of a heavy chain of the anti-PD-1 antibody (see FIG. 2B for exemplary structure), to construct IL-12/anti-PD-1 immunocytokines “Fab-IL-12-Fc-PD-1 Ab.” For example, Fab-IL-12(E59A/F60A)-Fc-PD-1 Ab immunocytokine (or “IL-12(E59A/F60A)/anti-PD-1 immunocytokine” or “construct #48”) comprising a single-chain IL-12 variant IL-12B (p40 E59A/F60A)-linker-IL-12A (wt p35) positioned at the hinge region comprises two light chains each comprising the amino acid sequence of SEQ ID NO: 106, one heavy chain comprising the amino acid sequence of SEQ ID NO: 107, and one heavy chain with the single-chain IL-12(E59A/F60A) variant (SEQ ID NO: 36) positioned at the hinge region comprising the amino acid sequence of SEQ ID NO: 112. IL-12(G64A)/anti-PD-1 immunocytokine (“construct #47”) comprising a single-chain IL-12 variant IL-12B (p40 G64A)-linker-IL-12A (wt p35) positioned at the hinge region comprises two light chains each comprising the amino acid sequence of SEQ ID NO: 106, one heavy chain comprising the amino acid sequence of SEQ ID NO: 107, and one heavy chain with the single-chain IL-12(G64A) variant (SEQ ID NO: 332) positioned at the hinge region. IL-12(E59A)/anti-PD-1 immunocytokine (“construct #46”) comprising a single-chain IL-12 variant IL-12B (p40 E59A)-linker-IL-12A (wt p35) positioned at the hinge region comprises two light chains each comprising the amino acid sequence of SEQ ID NO: 106, one heavy chain comprising the amino acid sequence of SEQ ID NO: 107, and one heavy chain with the single-chain IL-12(E59A) variant (SEQ ID NO: 331) positioned at the hinge region. Immunocytokines were constructed, expressed, and purified as described in Example 1.

### IL-12 Signal Transduction Assay

HEK-Blue™ IL-12 Cells (InvivoGen Cat. #hkb-i112) and HEK-PD-1-IL-12 cells (generated in-house by overexpressing human PD-1 in HEK-Blue™ IL-12 Cells using a lentiviral vector) were used to assess IL-12 signal activation activity of the various Fab-IL-12-Fc-PD-1 Ab immunocytokines comprising different IL-12 moieties, following the InvivoGen user manual (InvivoGen Cat. #hkb-il12), hereinafter also referred to as “HEK-IL-12 reporter assay” or “HEK-PD-1-IL-12 reporter assay.” HEK-Blue™ IL-12 reporter cells and HEK-PD-I-IL-12 reporter cells stably express the human IL-12 receptor complex consisting of the IL-12 receptor β1 (IL-12Rβ1) and IL-12Rβ2, along with the human STAT4 gene to obtain a fully functional IL-12 signaling pathway (TyK2/JAK2/STAT4). In addition, these reporter cells also carry a STAT4-inducible SEAP reporter gene. Upon IL-2 stimulation, HEK-Blue™ IL-12 reporter cells and HEK-PD-I-IL-12 reporter cells trigger the activation of STAT4 and the subsequent secretion of SEAP, the levels of which can be monitored using QUANTI-Blue™ (InvivoGen Cat. #rep-qbs) colorimetric enzyme assay for alkaline phosphatase activity. Experimental procedure was similar as described in Example 1 for IL-2 signal transduction assay. Recombinant human IL-12 (rIL-12) in free state served as positive control and reference for percent activity calculation.

TABLE 4
IL-12 biological activity of Fab-IL-12-Fc-PD-1
Abs comprising different IL-12 moieties

| | rIL-12 | “WT” | Q56A | V57A | K58A | E59A | F60A |
|---|---|---|---|---|---|---|---|
| HEK-IL-12 cells | 100.0% | 50.0% | 55.0% | 48.0% | 39.0% | 10.0% | 9.0% |
| HEK-PD-1-IL-12 cells | 100.0% | 120.0% | 150.0% | 91.0% | 98.0% | 45.0% | 56.0% |

| | G61A | D62A | A63S | G64A | Q65A | E59A/F60A |
|---|---|---|---|---|---|---|
| HEK-IL-12 cells | 56.0% | 57.0% | 62.0% | 69.0% | 62.0% | 0.1% |
| HEK-PD-1-IL-12 cells | 130.0% | 93.0% | 89.0% | 150.0% | 130.0% | 5.9% |

In HEK-IL-12 reporter assay, Fab-IL-12-Fc-PD-1 Abs were only able to bind to HEK-IL-12 cells via IL-12 moiety/IL-12 receptor interaction. Positioning IL-12 comprising wildtype p40 subunit at the hinge region of the anti-PD-1 antibody (“Fab-IL-12(WT)-Fc-PD-1 Ab”) reduced IL-12 activity to 50.0%, in the absence of PD-1 binding. As can be seen from Table 4, positions 59 and 60 of p40 subunit are crucial for IL-12 biological activity. Fab-IL-12-Fc-PD-1 Ab comprising E59A/F60A double mutations in the IL-12 p40 subunit (“Fab-IL-12(E59A/F60A)-Fc-PD-1 Ab”) showed almost completely aborted IL-12 activity as measured by IL-12 signal transduction (0.1%).

In HEK-PD-1-IL-12 reporter assay, Fab-IL-12-Fc-PD-1 Abs were able to bind to HEK-PD-1-IL-12 cells via both IL-12 moiety/IL-12 receptor interaction, and anti-PD-1 antigen-binding fragment/PD-1 interaction. As can be seen from Table 4, the biological activity of all IL-12 variants (and “WT” IL-12) in Fab-IL-12-Fc-PD-1 Ab increased with the presence of PD-1 binding. Especially, the IL-12 activity of Fab-IL-12(E59A/F60A)-Fc-PD-1 Ab was rescued by PD-1/anti-PD-1 antibody binding to 5.9%, which was 59-fold of that in the absence of PD-1/anti-PD-1 antibody binding (0.1%).

E59A/F60A double mutations in the IL-12 p40 subunit demonstrated superior effect compared to other mutations in IL-12 p40 subunit. By positioning this IL-12 E59A/F60A variant at the hinge region of a heavy chain of the anti-PD-1 full-length antibody, the obtained Fab-IL-12(E59A/F60A)-Fc-PD-1 Ab immunocytokine only exhibited IL-12 biological activity in the presence of target antigen (PD-1)-antibody binding, but not in the absence of target antigen (PD-1)-antibody binding, demonstrating targeted specificity.

### Example 5: Generation of IL-12/Anti-CD4 Immunocytokine (Fab-IL-12-Fc-CD4 Ab) with IL-12 Biological Activity Directed to CD4+ T Cells

### Construction of Fab-IL-12-Fc-CD4 Ab Immunocytokines

An anti-CD4 antibody comprising Ibalizumab (Trogarzo®) VH (SEQ ID NO: 73) and VL (SEQ ID NO: 74) sequences was used as the parental anti-CD4 full-length antibody, comprising two light chains each comprising the amino acid sequence of SEQ ID NO: 77. Ibalizumab is a non-immunosuppressive humanized monoclonal antibody that has been approved by FDA in HIV treatment. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Single chain “wildtype” IL-12, or IL-12 variant comprising E59A, F60A, or E59A/F60A double mutation in p40 subunit (SEQ ID NOs: 31-33) as described in Example 4, was positioned within the hinge region of a heavy chain of the anti-CD4 antibody (see FIG. 2B for exemplary structure), to construct IL-12/anti-CD4 immunocytokines “Fab-IL-12-Fc-CD4 Ab.” For example, Fab-IL-12(E59A/F60A)-Fc-CD4 Ab immunocytokine comprises two light chains each comprising the amino acid sequence of SEQ ID NO: 77, one heavy chain comprising the amino acid sequence of SEQ ID NO: 78, and one heavy chain with the single-chain IL-12 variant (SEQ ID NO: 36) positioned at the hinge region comprising the amino acid sequence of SEQ ID NO: 83. Immunocytokines were constructed, expressed, and purified as described in Example 1. FACS was used to confirm that Fab-IL-12-Fc-CD4 Abs were able to bind to CD4+ T cells, but not to CD8+ T cells (data not shown).

### Interferon-Gamma Release Assay (IGRA) for Measuring IL-12 or IL-23 Biological Activity

The biological activity of IL-12 or IL-23 can be measured by the amount of IFN-γ released from activated T cells. Binding of IL-12 to its receptor (heterodimeric receptor composed of IL-12R-β1 and IL-12R-β2 subunits) triggers a signaling pathway involving TyK2 (tyrosine kinase 2), JAK2 (Janus kinase 2) and STAT4 (signal transducer and activator of transcription 4) which results in the production of IFN-γ. CD4+ T cells or CD8+ T cells were isolated from PBMC, and stimulated by anti-CD3 antibody (1 μg/mL OKT3) in the presence of recombinant human IL-2 (30 units/mL) for 5 days. After 5 days, the activated CD4+ or CD8+ T cells (80,000 cells/well) were cultured in the presence of increasing concentrations of recombinant human IL-12 (rIL-12) or recombinant human IL-23 (rIL-23) (0, 0.62, 1.25, 2.5, 5, 10, or 20 ng/mL). The following day, the amount of IFN-γ released into the cell culture medium was measured by an ELISA assay. Percent IL-12 or IL-23 biological activity was calculated by dividing the readout of IL-12-immunocytokine or IL-23-immunocytokine by the readout of rIL-12 or rIL-23.

The minimal concentration of rIL-12 required to stimulate the release of IFN-γ from activated T cells was 2.5 ng/ml, while for rIL-23 it was 5 ng/ml. To determine the minimal concentration of the IL-12-immunocytokine or the IL-23-immunocytokine to observe a positive biological response, activated CD4+ or CD8+ T cells (80,000 cells/well) were cultured overnight in the presence of increasing concentrations of IL-12-immunocytokines or IL-23-immunocytokines (0, 0.32, 1.6, 8, 40, 200, or 1000 ng/mL). rIL-12 (2.5 ng/mL) or rIL-23 (5 ng/mL) served as positive control. The percent biological activity of the IL-12-immunocytokine or the IL-23-immunocytokine relative to corresponding free state cytokine (rIL-12 or rIL-23) was calculated by normalizing to corresponding molecular weights. The molecular weights of IL-12-immunocytokine and rIL-12 are about 220 kDa and about 70 kDa, respectively. The molecular weights of IL-23-immunocytokine and rIL-23 are about 215 kDa and about 65 kDa, respectively. Hence, about 3 ng of IL-12-immunocytokine or IL-23-immunocytokine is equivalent to about 1 ng of rIL-12 or rIL-23 for the same IL-12 or IL-23 molar concentration.

TABLE 5
IL-12 biological activity of Fab-IL-12-Fc-
CD4 Abs comprising different IL-12 moieties

| | rIL-12 | “WT” | E59A | F60A | E59A/F60A |
|---|---|---|---|---|---|
| CD4+ T cells | 100.0% | 43.0% | 29.8% | 32.2% | 8.2% |
| CD8+ T cells | 100.0% | 25.0% | 1.1% | 1.5% | 0.0% |

As can be seen from Table 5, positioning IL-12 comprising a wildtype p40 subunit at the hinge region still retained IL-12 activity of about 25.0%, even in the absence of target antigen (CD4)-antibody binding in CD8+ T cells. E59A and F60A mutations in p40 subunit significantly reduced IL-12 activity to about 1.1% or 1.5% in the absence of CD4/anti-CD4 antibody binding, which was rescued to about 29.8% or 32.2% in the presence of CD4+ T cells/anti-CD4 antibody binding. Fab-IL-12-Fc-CD4 Ab comprising E59A/F60A double mutations in IL-12 p40 subunit (“Fab-IL-12(E59A/F60A)-Fc-CD4 Ab”) demonstrated CD4+ T cells specific IL-12 biological activity (8.2%), with no cross reactivity with CD8+ T cells (0.0%). These data demonstrate successful generation of anti-CD4 antibody-based immunocytokines that can specifically target cytokine (e.g., IL-12) biological activity towards CD4+ T cells.

### Example 6: Generation of IL-12/Anti-CD8 Immunocytokine (Fab-IL-12-Fc-CD8 Ab) with IL-12 Biological Activity Directed to CD8+ T Cells

An anti-CD8 antibody comprising clone G10⁻¹ VH (SEQ ID NO: 114) and VL (SEQ ID NO: 115) sequences was used as the parental anti-CD8 full-length antibody, comprising two light chains each comprising the amino acid sequence of SEQ ID NO: 117. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Single chain “wildtype” IL-12, or IL-12 variant comprising E59A, F60A, or E59A/F60A double mutation in p40 subunit (SEQ ID NOs: 31-33) as described in Example 4, was positioned within the hinge region of a heavy chain of the anti-CD8 antibody (see FIG. 2B for exemplary structure), to construct IL-12/anti-CD8 immunocytokines “Fab-IL-12-Fc-CD8 Ab.” For example, Fab-IL-12(E59A/F60A)-Fc-CD8 Ab immunocytokine comprises two light chains each comprising the amino acid sequence of SEQ ID NO: 117, one heavy chain comprising the amino acid sequence of SEQ ID NO: 118, and one heavy chain with the single-chain IL-12 variant (SEQ ID NO: 36) positioned at the hinge region comprising the amino acid sequence of SEQ ID NO: 123. Immunocytokines were constructed, expressed, and purified as described in Example 1. FACS was used to confirm that Fab-IL-12-Fc-CD8 Abs were able to bind to CD8+ T cells, but not to CD4+ T cells (data not shown).

IFN-γ release assay was conducted similarly as described in Example 5, by culturing activated CD4+ or CD8+ T cells in the presence of increasing concentrations of rIL-12 (positive control) or Fab-IL-12-Fc-CD8 Abs. The following day, the amount of IFN-γ released into the cell culture medium was measured by an ELISA assay. Percent IL-12 biological activity was calculated by dividing the readout of Fab-IL-12-Fc-CD8 Ab by the readout of rIL-12.

TABLE 6
IL-12 biological activity of Fab-IL-12-Fc-
CD8 Abs comprising different IL-12 moieties

| | rIL-12 | “WT” | E59A | F60A | E59A/F60A |
|---|---|---|---|---|---|
| CD4+ T cells | 100.0% | 21.0% | 2.1% | 1.2% | 0.0% |
| CD8+ T cells | 100.0% | 56.0% | 24.2% | 21.9% | 11.0% |

As can be seen from Table 6, positioning IL-12 comprising a wildtype p40 subunit at the hinge region still retained IL-12 activity of about 21.0%, even in the absence of target antigen (CD8)-antibody binding in CD4+ T cells. E59A and F60A mutations in p40 subunit significantly reduced IL-12 activity to about 2.1% or 1.2% in the absence of CD8/anti-CD8 antibody binding, which was rescued to about 24.2% or 21.9% in the presence of CD8+ T cells/anti-CD8 antibody binding. Fab-IL-12-Fc-CD8 Ab comprising E59A/F60A double mutations in IL-12 p40 subunit (“Fab-IL-12(E59A/F60A)-Fc-CD8 Ab”) demonstrated CD8+ T cells specific IL-12 biological activity (11.0%), with no cross reactivity with CD4+ T cells (0.0%). These data demonstrate successful generation of anti-CD8 antibody-based immunocytokines that can specifically target cytokine (e.g., IL-12) biological activity towards CD8+ T cells.

### Example 7: Generation of IL-23/Anti-PD-1 Immunocytokine (Fab-IL-23-Fc-PD-1 Ab) with IL-23 Biological Activity Directed to PD-1-Positive Cells

### Construction of IL-23 Variants and Immunocytokines Thereof

IL-23 is a heterodimeric cytokine composed of p19 subunit and p40 subunit. The p40 subunit is shared with IL-12. IL-23 variants were constructed similarly as described in Example 4, by generating amino acid substitutions in the shared p40 subunit (see Table 7). A single chain IL-23 variant was made, from N′ to C′: p40 variant subunit (SEQ ID NOs: 31-34)—linker (SEQ ID NO: 229)—p19 wildtype subunit (SEQ ID NO: 37). A single chain “wildtype” IL-23 was also constructed as a control (SEQ ID NO: 38), from N′ to C′: p40 wildtype subunit-linker (SEQ ID NO: 229)—p19 wildtype subunit, referred to as “WT” in Table 7.

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Various single chain IL-23 variants (or single chain “wildtype” IL-23 control) were positioned within the hinge region of a heavy chain of the anti-PD-1 antibody (see FIG. 2B for exemplary structure), to construct IL-23/anti-PD-1 immunocytokines “Fab-IL-23-Fc-PD-1 Ab.” For example, Fab-IL-23(E59A/F60A)-Fc-PD-1 Ab immunocytokine comprising a single-chain IL-23 variant IL-12B (p40 E59A/F60A)-linker-IL-23A (wt p19) positioned at the hinge region comprises two light chains each comprising the amino acid sequence of SEQ ID NO: 106, one heavy chain comprising the amino acid sequence of SEQ ID NO: 107, and one heavy chain with the single-chain IL-23 variant (SEQ ID NO: 39) positioned at the hinge region comprising the amino acid sequence of SEQ ID NO: 113. Immunocytokines were constructed, expressed, and purified as described in Example 1.

### IL-23 Signal Transduction Assay

HEK-Blue™ IL-23 Cells (InvivoGen Cat. #hkb-i123) and HEK-PD-1-IL-23 cells (generated in-house by overexpressing human PD-1 in HEK-Blue™ IL-23 Cells using a lentiviral vector) were used to assess IL-23 signal activation activity of the various Fab-IL-23-Fc-PD-1 Ab immunocytokines comprising different IL-23 moieties, following the InvivoGen user manual (InvivoGen Cat. #hkb-i123), hereinafter also referred to as “HEK-IL-23 reporter assay” or “HEK-PD-1-IL-23 reporter assay.” HEK-Blue™ IL-23 reporter cells and HEK-PD-1-IL-23 reporter cells stably express the receptor complex consisting of IL-12R1 and the IL-23 receptor (IL-23R), along with the human STAT3 gene to obtain a fully functional IL-23 signaling pathway (TyK2/JAK2/STAT3). In addition, these reporter cells also carry a STAT3-inducible SEAP reporter gene. Upon IL-2 stimulation, HEK-Blue™ IL-23 reporter cells and HEK-PD-1-IL-23 reporter cells trigger the activation of STAT3 and the subsequent secretion of SEAP, the levels of which can be monitored using QUANTI-Blue™ (InvivoGen Cat. #rep-qbs) colorimetric enzyme assay for alkaline phosphatase activity. Experimental procedure was similar as described in Example 1 for IL-2 signal transduction assay. Recombinant human IL-23 (rIL-23) in free state served as positive control and reference for percent activity calculation.

TABLE 7
IL-23 biological activity of Fab-IL-23-Fc-PD-1
Abs comprising different IL-23 moieties

| | rIL-23 | “WT” | G64A | E59A | F60A | E59A/F60A |
|---|---|---|---|---|---|---|
| HEK-IL-23 cells | 100.0% | 70.0% | 56.0% | 6.9% | 8.2% | 0.0% |
| HEK-PD-1-IL-23 cells | 100.0% | 150.0% | 180.0% | 39.0% | 46.0% | 4.8% |

As can be seen from Table 7, positioning IL-23 comprising a wildtype p40 subunit at the hinge region retained IL-23 activity of about 70.0%, even in the absence of target antigen (PD-1)-antibody binding in HEK-IL-23 reporter cells. E59A and F60A mutations in p40 subunit significantly reduced IL-23 activity to about 6.9% or 8.2% in the absence of PD-1/anti-PD-1 antibody binding, which was rescued to about 39.0% or 46.0% in the presence of PD-1/anti-PD-1 antibody binding in HEK-PD-1-IL-23 cells. Fab-IL-23-Fc-PD-1 Ab comprising E59A/F60A double mutations in IL-23 p40 subunit (“Fab-IL-23(E59A/F60A)-Fc-PD-1 Ab”) demonstrated PD-1-positive cell specific IL-23 biological activity (4.8%), with no cross reactivity with PD-1-negative cells (0.0%). These data demonstrate successful generation of anti-PD-1 antibody-based immunocytokines that can specifically target cytokine (e.g., IL-23) biological activity towards PD-1+ cells.

### Example 8: Generation of IL-23/Anti-CD4 Immunocytokine (Fab-IL-23-Fc-CD4 Ab) with IL-23 Biological Activity Directed to CD4+ T Cells

An anti-CD4 antibody comprising Ibalizumab (Trogarzo®) VH (SEQ ID NO: 73) and VL (SEQ ID NO: 74) sequences was used as the parental anti-CD4 full-length antibody, comprising two light chains each comprising the amino acid sequence of SEQ ID NO: 77. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Single chain “wildtype” IL-23, or IL-23 variant comprising E59A, F60A, or E59A/F60A double mutation in p40 subunit (SEQ ID NOs: 31-33) as described in Example 7, was positioned within the hinge region of a heavy chain of the anti-CD4 antibody (see FIG. 2B for exemplary structure), to construct IL-23/anti-CD4 immunocytokines “Fab-IL-23-Fc-CD4 Ab.” For example, Fab-IL-23(E59A/F60A)-Fc-CD4 Ab immunocytokine comprises two light chains each comprising the amino acid sequence of SEQ ID NO: 77, one heavy chain comprising the amino acid sequence of SEQ ID NO: 78, and one heavy chain with the single-chain IL-23 variant (SEQ ID NO: 39) positioned at the hinge region comprising the amino acid sequence of SEQ ID NO: 84. Immunocytokines were constructed, expressed, and purified as described in Example 1. FACS was used to confirm that Fab-IL-23-Fc-CD4 Abs were able to bind to CD4+ T cells, but not to CD8+ T cells (data not shown).

IL-23 can stimulate activated CD4+ or CD8+ T cells to release IFN-γ. The IL-23 biological activity of various Fab-IL-23-Fc-CD4 Abs was measured using the IFN-γ release assay as described in Example 5. rIL-23 served as positive control and percent activity reference.

TABLE 8
IL-23 biological activity of Fab-IL-23-Fc-
CD4 Abs comprising different IL-23 moieties

| | rIL-23 | “WT” | E59A | F60A | E59A/F60A |
|---|---|---|---|---|---|
| CD4+ T cells | 100.0% | 34.0% | 24.0% | 29.0% | 6.8% |
| CD8+ T cells | 100.0% | 21.0% | 2.0% | 1.5% | 0.0% |

As can be seen from Table 8, positioning IL-23 comprising a wildtype p40 subunit at the hinge region still retained IL-23 activity of about 21.0%, even in the absence of target antigen (CD4)-antibody binding in CD8+ T cells. E59A and F60A mutations in p40 subunit significantly reduced IL-23 activity to about 2.0% or 1.5% in the absence of CD4/anti-CD4 antibody binding, which was rescued to about 24.0% or 29.0% in the presence of CD4+ T cells/anti-CD4 antibody binding. Fab-IL-23-Fc-CD4 Ab comprising E59A/F60A double mutations in IL-23 p40 subunit (“Fab-IL-23(E59A/F60A)-Fc-CD4 Ab”) demonstrated CD4+ T cells specific IL-23 biological activity (6.8%), with no cross reactivity with CD8+ T cells (0.0%). These data demonstrate successful generation of anti-CD4 antibody-based immunocytokines that can specifically target cytokine (e.g., IL-23) biological activity towards CD4+ T cells.

### Example 9: Generation of IL-10/Anti-PD-1 Immunocytokine (Fab-IL-10-Fc-PD-1 Ab) with IL-10 Biological Activity Directed to PD-1-Positive Cells

### Construction of IL-10 Variants and Immunocytokines Thereof

IL-10 is naturally expressed as a non-covalently linked homodimer. IL-10 variants were constructed by replacing amino acids from position 24 to 32 with Alanine or Serine (see Table 9), and a single chain IL-10 variant was made, from N′ to C′: IL-10 variant subunit (SEQ ID NOs: 21-26)-linker (SEQ ID NO: 227)—IL-10 variant subunit. A single chain “wildtype” IL-10 was also constructed as a control (SEQ ID NO: 27), from N′ to C′: IL-10 wildtype subunit (SEQ ID NO: 20) —linker (SEQ ID NO: 227)—IL-10 wildtype subunit, referred to as “WT” in Table 9.

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Various single chain IL-10 variants (or single chain “wildtype” IL-10 control) were positioned within the hinge region of a heavy chain of the anti-PD-1 antibody (see FIG. 2A for exemplary structure), to construct IL-10/anti-PD-1 immunocytokines “Fab-IL-10-Fc-PD-1 Ab.” For example, Fab-IL-I0(R27A)-Fc-PD-1 Ab immunocytokine comprising a single-chain IL-10 variant IL-I0(R27A)-linker-IL-I0(R27A) positioned at the hinge region comprises two light chains each comprising the amino acid sequence of SEQ ID NO: 106, one heavy chain comprising the amino acid sequence of SEQ ID NO: 107, and one heavy chain with the single-chain IL-10 variant (SEQ ID NO: 28) positioned at the hinge region comprising the amino acid sequence of SEQ ID NO: 111. Immunocytokines were constructed, expressed, and purified as described in Example 1.

### IL-10 Signal Transduction Assay

HEK-Blue™ IL-10 Cells (InvivoGen Cat. #hkb-il10) and HEK-PD-1-IL-10 cells (generated in-house by overexpressing human PD-1 in HEK-Blue™ IL-10 Cells using a lentiviral vector) were used to assess IL-10 signal activation activity of the various Fab-IL-10-Fc-PD-1 Abs comprising different IL-10 moieties, following the InvivoGen user manual (InvivoGen Cat. #hkb-il10), hereinafter also referred to as “HEK-IL-10 reporter assay” or “HEK-PD-I-IL-10 reporter assay.” HEK-Blue™ IL-10 reporter cells and HEK-PD-I-IL-10 reporter cells stably express IL-10 receptor hIL-10Ra and hIL-10RD chains, human STAT3, and STAT3-inducible SEAP. Binding of IL-10 to its receptor on the surface of HEK-Blue™ IL-10 cells or HEK-PD-I-IL-23 reporter cells triggers JAK1/STAT3 signaling and the subsequent production of SEAP, the level of which in the cell culture supernatant can be monitored using QUANTI-Blue™ (InvivoGen Cat. #rep-qbs). Experimental procedure was similar as described in Example 1 for IL-2 signal transduction assay. Recombinant human IL-10 (rIL-10) in free state served as positive control and reference for percent activity calculation.

TABLE 9
IL-10 biological activity of Fab-IL-10-Fc-PD-1
Abs comprising different IL-10 moieties

| | rIL-10 | “WT” | R24A | D25A/L26A |
|---|---|---|---|---|
| HEK-IL-10 cells | 100.0% | 26.0% | 15.0% | 4.0% |
| HEK-PD-1-IL-10 cells | 100.0% | 200.0% | 150.0% | 56.0% |
| | R27A | D28A/A29S | F30A/S31A | R32A |
| HEK-IL-10 cells | <0.1% | 17.0% | 13.0% | 10.0% |
| HEK-PD-1-IL-10 cells | 21.0% | 70.0% | 67.0% | 42.0% |

As can be seen from Table 9, positioning IL-10 comprising wildtype IL-10 subunit at the hinge region still retained IL-10 activity of about 26.0%, even in the absence of target antigen (PD-1)-antibody binding in HEK-IL-10 cells. All IL-10 variants tested reduced IL-10 activity in the absence of PD-1/anti-PD-1 antibody binding compared to that of “wildtype” IL-10, and their IL-10 activity was rescued in the presence of PD-1/anti-PD-1 antibody binding in HEK-PD-I-IL-10 cells. Fab-IL-10-Fc-PD-1 Ab comprising R27A mutation in IL-10 (“Fab-IL-10(R27A)-Fc-PD-1 Ab”) demonstrated PD-1-positive cell specific IL-10 biological activity (21.0%), with minimal cross reactivity with PD-1-negative cells (≤0.1%). These data demonstrate successful generation of anti-PD-1 antibody-based immunocytokines that can specifically target cytokine (e.g., IL-10) biological activity towards PD-1-positive cells.

### Example 10: Generation of IFN-γ/Anti-PD-1 Immunocytokine (Fab-IFN-γ-Fc-PD-1 Ab) with IFN-γ Biological Activity Directed to PD-1-Positive Cells

### Construction of IFN-γ Variants and Immunocytokines Thereof

IFN-γ is naturally expressed as a symmetric homodimer. IFN-γ variants were constructed by replacing amino acids from position 20 to 25 with A, K, S, E, Q, or V (see Table 10), and a single chain IFN-γ variant was made, from N′ to C′: IFN-γ variant subunit (SEQ ID NOs: 11-17)—linker (SEQ ID NO: 227)—IFN-γ variant subunit. A single chain “wildtype” IFN-γ was also constructed as a control (SEQ ID NO: 18), from N′ to C′: IFN-γ wildtype subunit (SEQ ID NO: 10)-linker (SEQ ID NO: 227)—IFN-γ wildtype subunit, referred to as “WT” in Table 10.

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Various single chain IFN-γ variants (or single chain “wildtype” IFN-γ control) were positioned within the hinge region of a heavy chain of the anti-PD-1 antibody (see FIG. 2A for exemplary structure), to construct IFN-γ/anti-PD-1 immunocytokines “Fab-IFN-γ-Fc-PD-1 Ab.” For example, Fab-IFN-γ(A23V)-Fc-PD-1 Ab immunocytokine comprising a single-chain IFN-γ variant IFN-γ(A23V)-linker-IFN-γ(A23V) positioned at the hinge region comprises two light chains each comprising the amino acid sequence of SEQ ID NO: 106, one heavy chain comprising the amino acid sequence of SEQ ID NO: 107, and one heavy chain with the single-chain IFN-γ variant (SEQ ID NO: 19) positioned at the hinge region comprising the amino acid sequence of SEQ ID NO: 110. Immunocytokines were constructed, expressed, and purified as described in Example 1.

### IFN-γ Signal Transduction Assay

HEK-Blue™ IFN-γ Cells (InvivoGen Cat. #hkb-ifng) and HEK-PD-1-IFN-γ cells (generated in-house by overexpressing human PD-1 in HEK-Blue™ IFN-γ Cells using a lentiviral vector) were used to assess IFN-γ signal activation activity of the various Fab-IFN-γ-Fc-PD-1 Abs comprising different IFN-γ moieties, following the InvivoGen user manual (InvivoGen Cat. #hkb-ifng), hereinafter also referred to as “HEK-IFN-γ reporter assay” or “HEK-PD-1-IFN-γ reporter assay.” HEK-Blue™ IFN-γ reporter cells and HEK-PD-1-IFN-γ reporter cells stably express human STAT1 gene, and STAT1-inducible SEAP. The other genes of the pathway are naturally expressed in sufficient amounts in the reporter cells. Binding of IFN-γ to its heterodimeric receptor consisting of IFNGR1 and IFNGR2 chains on the surface of HEK-Blue™ IFN-γ cells or HEK-PD-1-IFN-γ reporter cells triggers JAK1/JAK2/STAT1 signaling and the subsequent production of SEAP, the level of which in the cell culture supernatant can be monitored using QUANTI-Blue™ (InvivoGen Cat. #rep-qbs). Experimental procedure was similar as described in Example 1 for IL-2 signal transduction assay. Recombinant human IFN-γ (rIFN-γ) in free state served as positive control and reference for percent activity calculation.

TABLE 10
IFN-γ biological activity of Fab-IFN-γ-Fc-PD-1
Abs comprising different IFN-γ moieties

| | rIFN-γ | “WT” | S20A/D21A | D21K | V22A/A23S |
|---|---|---|---|---|---|
| HEK-IFN-γ cells | 100.0% | 36.0% | 30.0% | 35.0% | 1.2% |
| HEK-PD-1-IFN-γ cells | 100.0% | 130.0% | 89.0% | 110.0% | 24.0% |

| | D24A/N25A | A23E/D24E/N25K | A23Q | A23V |
|---|---|---|---|---|
| HEK-IFN-γ cells | 21.0% | 0.2% | 0.8% | 0.6% |
| HEK-PD-1-IFN-γ cells | 70.0% | 23.0% | 31.0% | 27.0% |

As can be seen from Table 10, positioning IFN-γ comprising wildtype IFN-γ subunit at the hinge region retained IFN-γ activity of about 36.0%, even in the absence of target antigen (PD-1)-antibody binding in HEK-IFN-γ cells. A23 residue appears critical for IFN-γ biological activity, as all IFN-γ variants comprising A23 mutation greatly reduced IFN-γ activity in the absence of PD-1/anti-PD-1 antibody binding compared to that of “wildtype” IFN-γ, and their IFN-γ activity was rescued in the presence of PD-1/anti-PD-1 antibody binding in HEK-PD-1-IFN-γ cells. Fab-IFN-γ-Fc-PD-1 Ab comprising A23V mutation in IFN-γ (“Fab-IFN-γ(A23V)-Fc-PD-1 Ab”) demonstrated PD-1-positive cell specific IFN-γ biological activity (27.0%), with minimal cross reactivity with PD-1-negative cells (0.6%). Fab-IFN-γ-Fc-PD-1 Ab comprising A23E/D24E/N25K triple mutations in IFN-γ (“Fab-IFN-γ(A23E/D24E/N25K)-Fc-PD-1 Ab”) demonstrated PD-1-positive cell specific IFN-γ biological activity (23.0%), with minimal cross reactivity with PD-1-negative cells (0.2%). These data demonstrate successful generation of anti-PD-1 antibody-based immunocytokines that can specifically target cytokine (e.g., IFN-γ) biological activity towards PD-1-positive cells.

### Example 11: Generation of IFN-γ/Anti-CD4 Immunocytokine (Fab-IFN-γ-Fc-CD4 Ab) with IFN-γ Biological Activity Directed to CD4+ T Cells

An anti-CD4 antibody comprising Ibalizumab (Trogarzo®) VH (SEQ ID NO: 73) and VL (SEQ ID NO: 74) sequences was used as the parental anti-CD4 full-length antibody, comprising two light chains each comprising the amino acid sequence of SEQ ID NO: 77. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Single chain “wildtype” IFN-γ, or IFN-γ variant comprising various mutations as described in Example 10, was positioned within the hinge region of a heavy chain of the anti-CD4 antibody (see FIG. 2B for exemplary structure), to construct IFN-γ/anti-CD4 immunocytokines “Fab-IFN-γ-Fc-CD4 Ab.” For example, Fab-IFN-γ(A23V)-Fc-CD4 Ab immunocytokine comprises two light chains each comprising the amino acid sequence of SEQ ID NO: 77, one heavy chain comprising the amino acid sequence of SEQ ID NO: 78, and one heavy chain with the single-chain IFN-γ variant (SEQ ID NO: 19) positioned at the hinge region comprising the amino acid sequence of SEQ ID NO: 81. Immunocytokines were constructed, expressed, and purified as described in Example 1.

IFN-γ can induce PD-L1 expression on cell surface. To assess biological activity of various IFN-γ moiety within the Fab-IFN-γ-Fc-CD4 Ab, HEK-CD4-IFN-γ cells were generated in-house by overexpressing human CD4 in HEK-Blue™ IFN-γ Cells (InvivoGen Cat. #hkb-ifng) using a lentiviral vector. rIFN-γ served as positive control. Anti-PD-1 antibody nivolumab (Opdivo®) and no IFN-γ treatment served as negative controls. Briefly, HEK-Blue™ IFN-γ Cells or HEK-CD4-IFN-γ cells were cultured in the presence of culture medium only (no IFN-γ), rIFN-γ (20 ng/mL), anti-PD-1 antibody (1 μg/mL), or various Fab-IFN-γ-Fc-CD4 Abs (1 μg/mL) at 37° C. in a C02 incubator overnight. The following day, cells were stained with anti-human PD-L1 Allophycocyanin (APC)-conjugated antibody (BioLegend, Cat. #374513) to detect PD-L1 expression. The binding activity of different Fab-IFN-γ-Fc-CD4 Abs (or controls) to HEK-Blue™ IFN-γ Cells or HEK-CD4-IFN-γ cells was reflected as mean fluorescence intensity (MFI) measured using FACS.

TABLE 11
IFN-γ biological activity of Fab-IFN-γ-Fc-CD4
Abs comprising different IFN-γ moieties

| (unit: MFI) | No IFN-γ | rIFN-γ | Anti-PD-1 Ab | “WT” | V22A/A23S |
|---|---|---|---|---|---|
| HEK-IFN-γ cells | 15 | 75 | 14 | 57 | 18 |
| HEK-CD4-IFN-γ cells | 13 | 67 | 15 | 85 | 68 |

| (unit: MFI) | D24A/N25A | A23E/D24E/N25K | A23Q | A23V |
|---|---|---|---|---|
| HEK-IFN-γ cells | 63 | 13 | 17 | 12 |
| HEK-CD4-IFN-γ cells | 81 | 59 | 72 | 68 |

As can be seen from Table 11, no IFN-γ treatment or anti-PD-1 antibody (negative control) treatment of reporter cells showed base line PD-L1 expression of ˜15 MFI. Positioning “WT” IFN-γ comprising wildtype IFN-γ subunit at the hinge region of the anti-CD4 antibody retained IFN-γ activity of about 57 MFI (compared to free state rIFN-γ of 75 MFI), in the absence of target antigen (CD4)-antibody binding in HEK-IFN-γ cells. Consistent with data in Example 10, A23 residue appears critical for IFN-γ biological activity, as all IFN-γ variants comprising A23 mutation greatly reduced IFN-γ activity close to baseline level (˜15 MFI) in the absence of CD4/anti-CD4 antibody binding compared to that of “WT” IFN-γ or D24A/N25A IFN-γ variant, and their IFN-γ activity was rescued in the presence of CD4/anti-CD4 antibody binding in HEK-CD4-IFN-γ cells. Fab-IFN-γ-Fc-CD4 Ab comprising A23E/D24E/N25K triple mutations in IFN-γ (“Fab-IFN-γ(A23E/D24E/N25K)-Fc-CD4 Ab”) or A23V mutation (“Fab-IFN-γ(A23V)-Fc-CD4 Ab”) demonstrated CD4+ cell specific IFN-γ biological activity (59 MFI or 68 MFI, respectively), with no or little cross reactivity with CD4-negative cells (13 MFI or 12 MFI, respectively, at or below baseline). These data demonstrate successful generation of anti-CD4 antibody-based immunocytokines that can specifically target cytokine (e.g., IFN-γ) biological activity towards CD4-positive cells.

### Example 12: Generation of IFN-α2b/Anti-PD-1 Immunocytokine (Fab-IFN-α2b-Fc-PD-1 Ab) with IFN-α2b Biological Activity Directed to PD-1-Positive Cells

### Construction of IFN-α2b Variants and Immunocytokines Thereof

IFN-α2b (Intron-A®) is an antiviral or antineoplastic drug. It is a recombinant form of IFN-α2. IFN-α2b variants were constructed by replacing amino acids at positions 30 and 32-34 with Alanine (SEQ ID NOs: 4, 6, 7, and 8; see Table 12). An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. Various IFN-α2b variants (or wildtype IFN-α2b control “WT”) were positioned within the hinge region of a heavy chain of the anti-PD-1 antibody (see FIG. 2A for exemplary structure), to construct IFN-α2b/anti-PD-1 immunocytokines “Fab-IFN-α2b-Fc-PD-1 Ab.” For example, the Fab-IFN-α2b(L30A)-Fc-PD-1 Ab immunocytokine comprises two light chains each comprising the amino acid sequence of SEQ ID NO: 106, one heavy chain comprising the amino acid sequence of SEQ ID NO: 107, and one heavy chain with the IFN-α2b(L30A) variant (SEQ ID NO: 4) positioned at the hinge region comprising the amino acid sequence of SEQ ID NO: 109. Immunocytokines were constructed, expressed, and purified as described in Example 1.

### IFN-α/β Signal Transduction Assay

HEK-Blue™ IFN-α/P cells (InvivoGen Cat. #hkb-ifnab) and HEK-PD-I-IFN-α/P cells (generated in-house by overexpressing human PD-1 in HEK-Blue™ IFN-α/β cells using a lentiviral vector) were used to assess IFN-α2b signal activation activity of the various Fab-IFN-α2b-Fc-PD-1 Abs comprising different IFN-α2b moieties, following the InvivoGen user manual (InvivoGen Cat. #hkb-ifnab), hereinafter also referred to as “HEK-IFN-α/D reporter assay” or “HEK-PD-1-IFN-α/β reporter assay.” HEK-Blue™ IFN-α/β reporter cells and HEK-PD-I-IFN-α/β reporter cells were generated by stable transfection of HEK293 cells with the human STAT2 and IRF9 genes to obtain a fully active type I IFN signaling pathway, and inducible SEAP under the control of IFN-α/β inducible ISG54 promoter. The other genes of the pathway (IFNAR1, IFNAR2, JAK1, TyK2, and STAT1) are naturally expressed by these cells. Binding of IFN-α or IFN-β to its heterodimeric receptor consisting of IFNAR1 and IFNAR2 chains triggers JAK/STAT/ISGF3 signaling and subsequent production of SEAP, the level of which in the cell culture supernatant can be monitored using QUANTI-Blue™ (InvivoGen Cat. #rep-qbs). Experimental procedure was similar as described in Example 1 for IL-2 signal transduction assay. IFN-α2b in free state served as positive control and reference for percent activity calculation.

TABLE 12
IFN-α2b biological activity of Fab-IFN-α2b-Fc-PD-1
Abs comprising different IFN-α2b moieties

| | IFN-α2b | | | | | |
|---|---|---|---|---|---|---|
| | (free state) | “WT” | L30A | D32A | R33A | H34A |
| HEK-IFN-α/β cells | 100.0% | 54.0% | 15.0% | 9.0% | 5.0% | 20.0% |
| HEK-PD-1-IFN-α/β cells | 100.0% | 96.0% | 110.0% | 50.0% | 25.0% | 56.0% |

As can be seen from Table 12, positioning wildtype IFN-α2b at the hinge region of the anti-PD-1 antibody retained IFN-α2b activity of about 54.0%, even in the absence of target antigen (PD-1)-antibody binding in HEK-IFN-α/D cells. L30, D32, R33, and H34 residues all appear critical for IFN-α2b biological activity, as all IFN-α2b variants greatly reduced IFN-α2b activity in the absence of PD-1/anti-PD-1 antibody binding compared to that of wildtype IFN-α2b, and their IFN-α2b activity was rescued in the presence of PD-1/anti-PD-1 antibody binding in HEK-PD-I-IFN-α/p cells. Fab-IFN-α2b-Fc-PD-1 Ab comprising L30A mutation in IFN-α2b (“Fab-IFN-α2b(L30A)-Fc-PD-1 Ab”) demonstrated PD-1-positive cell specific IFN-α2b biological activity (110.0%), with greatly reduced cross reactivity with PD-1-negative cells (15.0%). Fab-IFN-α2b-Fc-PD-1 Ab comprising R33A mutation in IFN-α2b (“Fab-IFN-α2b(R33A)-Fc-PD-1 Ab”) demonstrated PD-1-positive cell specific IFN-α2b biological activity (25.0%), with greatly reduced cross reactivity with PD-1-negative cells (5.0%). These data demonstrate successful generation of anti-PD-1 antibody-based immunocytokines that can specifically target cytokine (e.g., IFN-α2b) biological activity towards PD-1-positive cells, with reduced cytokine biological activity towards PD-1-negative cells.

### Example 13: Placing IL-2 Variant at the Hinge Region of the IL-2/PD-L2-Fc Immunocytokine Significantly Reduces Toxicity in Mice

Similarly as described in Example 3, a PD-L2-hinge-Fc fusion protein (two PD-L2-hinge-Fc polypeptides each comprising SEQ ID NO: 177) was used as parental antigen-binding protein to construct immunocytokines that bind to PD-1. To construct heterodimeric PD-L2-hinge-Fc fusion protein, one PD-L2-hinge-Fc fusion polypeptide comprises a hinge region comprising SEQ ID NO: 52, and an Fc domain subunit comprising SEQ ID NO: 61; the other PD-L2-Fc fusion polypeptide comprises a hinge region comprising SEQ ID NO: 51, and an Fc domain subunit comprising SEQ ID NO: 62. An IL-2 variant (R38D/K43E/E61R; SEQ ID NO: 2) was either positioned at the hinge region of one PD-L2-hinge-Fc polypeptide (hereinafter referred to as “IL-2(R38D/K43E/E61R)/PD-L2-Fc immunocytokine” or “construct #11”), or fused to the C-terminus of one PD-L2-hinge-Fc polypeptide (hereinafter referred to as “PD-L2-Fc/IL-2(R38D/K43E/E61R) immunocytokine”). IL-2(R38D/K43E/E61R)/PD-L2-Fc immunocytokine comprises one IL-2 fusion polypeptide comprising SEQ ID NO: 180 and one pairing polypeptide comprising SEQ ID NO: 179.

25 BALB/c mice were randomly divided into 5 groups (5 mice each group), and intraperitoneally injected with 200 μg or 1000 μg of IL-2(R38D/K43E/E61R)/PD-L2-Fc immunocytokine, PD-L2-Fc/IL-2(R38D/K43E/E61R) immunocytokine, or parental PD-L2-hinge-Fc fusion protein as control. Each group received intraperitoneal injections on Day 1 and Day 5. Mice were monitored daily for four parameters: i) fur texture, ii) reduced activity, iii) morbidity, and iv) weight loss greater than 10%.

As can be seen from Table 13, when placing IL-2 variant at the C-terminus of the PD-L2-hinge-Fc polypeptide, five out of five mice died 4-6 days post injection, in both high and low dose groups. In contrast, when IL-2 variant was positioned at the hinge region, all mice survived, even administered with high dose of immunocytokines (1000 μg). Same survival rate was observed in the control group (PD-L2-Fc without IL-2 fusion). For immunocytokines with IL-2 variant positioned at the hinge region, toxicity appeared to be dose-dependent, as indicated by increased weight loss and greater reduced activity when dose was increased from 200 μg to 1000 μg.

TABLE 13
In vivo toxicity of IL-2/PD-L2-Fc immunocytokines of different formats

| | Dose | Deaths | |
|---|---|---|---|
| Construct | (μg) | in group | Toxicity symptoms |
| IL-2(R38D/K43E/E61R)/ | 200 | None | Fur texture, reduced activity |
| PD-L2-Fc immunocytokine | 1000 | None | Fur texture, reduced activity, |
| (IL-2 positioned at | | | weight loss |
| hinge region; | | | |
| construct #11) | | | |
| PD-L2-Fc/IL-2(R38D/ | 200 | 5/5 | Fur texture, reduced activity, |
| K43E/E61R) immunocytokine | | | weight loss, morbidity |
| (IL-2 fused to the | 1000 | 5/5 | Fur texture, reduced activity, |
| C′ of Fc) | | | weight loss, morbidity |
| Parental PD-L2-hinge-Fc | 1000 | None | None |
| fusion protein (control) | | | |

### Example 14: Placing IL-12 Variant at the Hinge Region of the IL-12/Anti-PD-1 Immunocytokine Significantly Reduces Toxicity in Mice

### Construction of IL-12 Variants and Immunocytokines Thereof

Similarly as described in Example 4, 3 single chain IL-12 variants were generated. “IL-12 (F60A)” (SEQ ID NO: 275) comprises from N′ to C′: p40 F60A variant subunit (SEQ ID NO: 33)—linker (SEQ ID NO: 228)—p35 wildtype subunit (SEQ ID NO: 29). “IL-12 (G64A)” comprises from N′ to C′: p40 F60A variant subunit (SEQ ID NO: 34)—linker (SEQ ID NO: 228)—p35 wildtype subunit (SEQ ID NO: 29). “IL-12 (E59A/F60A)” (SEQ ID NO: 36) comprises from N′ to C′: p40 E59A/F60A variant subunit (SEQ ID NO: 31)—linker (SEQ ID NO: 228) —p35 wildtype subunit (SEQ ID NO: 29).

As described in Example 4, IL-12(E59A/F60A)/anti-PD-1 immunocytokine (construct #48) comprises two light chains each comprising the sequence of SEQ ID NO: 106, one heavy chain comprising the sequence of SEQ ID NO: 107, and one heavy chain with the single-chain IL-12(E59A/F60A) variant (SEQ ID NO: 36) positioned at the hinge region comprising the amino acid sequence of SEQ ID NO: 112. The single-chain IL-12(E59A/F60A) variant within IL-12(E59A/F60A)/anti-PD-1 immunocytokine was replaced with either single-chain IL-12 (F60A) variant (SEQ ID NO: 275) or single-chain IL-12 (G64A) variant (SEQ ID NO: 332) to construct IL-12(F60A)/anti-PD-1 immunocytokine and IL-12(G64A)/anti-PD-1 immunocytokine, respectively. Immunocytokines were constructed, expressed, and purified as described in Example 1.

30 BALB/c mice were randomly divided into 6 groups (5 mice each group), and intraperitoneally injected with 200 μg or 1000 μg of IL-12(F60A)/anti-PD-1 immunocytokine, IL-12(G64A)/anti-PD-1 immunocytokine, or IL-12(E59A/F60A)/anti-PD-1 immunocytokine. Each group received intraperitoneal injections on Day 1 and Day 5. Mice were monitored daily for four parameters: i) fur texture, ii) reduced activity, iii) morbidity, and iv) weight loss greater than 10%.

As can be seen from Table 14, IL-12/anti-PD-1 immunocytokine comprising IL-12 (G64A) variant showed the highest toxicity, as indicated by the death of 4/5 mice in low dose group and the death of 5/5 mice in high dose group. In contrast, treatment with IL-12/anti-PD-1 immunocytokine comprising IL-12 (F60A) variant only induced one death in high dose group (1000 μg) and no death in low dose group (200 μg); treatment with IL-12/anti-PD-1 immunocytokine comprising IL-12 (E59A/F60A) variant did not induce death in either dose. IL-12/anti-PD-1 immunocytokine comprising IL-12 double mutation E59A/F60A also demonstrated less toxicity compared to that comprising IL-12 single F60A mutation, as indicated by the differences in severity of toxicity symptoms.

TABLE 14
In vivo toxicity of IL-12/anti-PD-1 immunocytokines

| | Dose | Deaths | |
|---|---|---|---|
| Construct | (μg) | in group | Toxicity symptoms |
| IL-12(F60A)/anti-PD-1 | 200 | None | Fur texture, reduced activity |
| immunocytokine | 1000 | 1/5 | Fur texture, reduced activity, |
| | | | weight loss, morbidity |
| IL-12(G64A)/anti-PD-1 | 200 | 4/5 | Fur texture, reduced activity, |
| immunocytokine | | | weight loss, morbidity |
| (construct #47) | 1000 | 5/5 | Fur texture, reduced activity, |
| | | | weight loss, morbidity |
| IL-12(E59A/F60A)/anti- | 200 | None | Fur texture (moderate) |
| PD-1 immunocytokine | | | |
| (construct #48) | 1000 | None | Fur texture, reduced activity |

### Example 15: Replacing Anti-PD-1 Parental Antibody with PD-L2-Hinge-Fc Fusion Protein Significantly Reduces Toxicity of IL-12 Immunocytokines

### Construction of IL-12/PD-L2-Fc Immunocytokines

A PD-L2-hinge-Fc fusion protein (two PD-L2-hinge-Fc polypeptides each comprising SEQ ID NO: 177) was used as parental antigen-binding protein to construct immunocytokines that bind to PD-1. To construct heterodimeric PD-L2-hinge-Fc fusion protein, one PD-L2-hinge-Fc fusion polypeptide comprises a hinge region comprising SEQ ID NO: 52, and an Fc domain subunit comprising SEQ ID NO: 61; the other PD-L2-Fc fusion polypeptide comprises a hinge region comprising SEQ ID NO: 51, and an Fc domain subunit comprising SEQ ID NO: 62. Single chain IL-12 variant (F60A, G64A, or E59A/F60A) as described in Example 14 was either positioned at the hinge region of one PD-L2-hinge-Fc polypeptide (hereinafter referred to as “IL-12/PD-L2-Fc immunocytokine”), or fused to the C-terminus of one PD-L2-hinge-Fc polypeptide (hereinafter referred to as “PD-L2-Fc/IL-12 immunocytokine”). For example, IL-12(E59A/F60A)/PD-L2-Fc immunocytokine (“construct #29”) comprises one IL-12 fusion polypeptide comprising SEQ ID NO: 186 (from N′ to C′: PD-L2 extracellular domain (SEQ ID NO: 176)—GSG linker (SEQ ID NO: 203)—single chain IL-12(E59A/F60A) variant (SEQ ID NO: 36)—hinge (SEQ ID NO: 52)—Fc domain subunit (SEQ ID NO: 61)); and one pairing polypeptide comprising SEQ ID NO: 185 (from N′ to C′: PD-L2 extracellular domain (SEQ ID NO: 176)—GSG linker—hinge (SEQ ID NO: 51)—Fc domain subunit (SEQ ID NO: 62)). The single chain IL-12(E59A/F60A) variant within IL-12(E59A/F60A)/PD-L2-Fc immunocytokine was replaced with either single-chain IL-12 (F60A) variant (SEQ ID NO: 275) or single-chain IL-12 (G64A) variant (SEQ ID NO: 332) to construct IL-12(F60A)/PD-L2-Fc immunocytokine (“construct #30”) and IL-12(G64A)/PD-L2-Fc immunocytokine, respectively. For example, IL-12(F60A)/PD-L2-Fc immunocytokine comprises one IL-12 fusion polypeptide comprising SEQ ID NO: 274 (from N′ to C′: PD-L2 extracellular domain (SEQ ID NO: 176)—GSG linker (SEQ ID NO: 203)—single chain IL-12(F60A) variant (SEQ ID NO: 275)—hinge (SEQ ID NO: 52) —Fc domain subunit (SEQ ID NO: 61)); and one pairing polypeptide comprising SEQ ID NO: 185 (from N′ to C′: PD-L2 extracellular domain (SEQ ID NO: 176)—GSG linker (SEQ ID NO: 203)—hinge (SEQ ID NO: 51)—Fc domain subunit (SEQ ID NO: 62)). PD-L2-Fc/IL-12(F60A) immunocytokine (“construct #34”) comprises one IL-12 fusion polypeptide comprising SEQ ID NO: 276 (from N′ to C′: PD-L2 extracellular domain (SEQ ID NO: 176)—GSG linker (SEQ ID NO: 203)—hinge (SEQ ID NO: 52)—Fc domain subunit (SEQ ID NO: 61) —GGGGSGGGGSGGGGS linker (SEQ ID NO: 229)—single chain IL-12(F60A) variant (SEQ ID NO: 275)); and one pairing polypeptide comprising SEQ ID NO: 185 (from N′ to C′: PD-L2 extracellular domain (SEQ ID NO: 176)—GSG linker (SEQ ID NO: 203)—hinge (SEQ ID NO: 51)—Fc domain subunit (SEQ ID NO: 62)). Immunocytokines were constructed, expressed, and purified as described in Example 1.

50 BALB/c mice were randomly divided into 10 groups (5 mice each group), and intraperitoneally injected with 200 μg or 1000 μg of: i) IL-12(F60A)/PD-L2-Fc immunocytokine, ii) IL-12(G64A)/PD-L2-Fc immunocytokine, iii) IL-12(E59A/F60A)/PD-L2-Fc immunocytokine, iv) PD-L2-Fc/IL-12(F60A) immunocytokine (IL-12 at C′ of Fc), or v) PD-L2-Fc/IL-12(E59A/F60A) immunocytokine (IL-12 at C′ of Fc). Each group received intraperitoneal injections on Day 1 and Day 5. Mice were monitored daily for four parameters: i) fur texture, ii) reduced activity, iii) morbidity, and iv) weight loss greater than 10%.

As can be seen from Table 15, among immunocytokines with IL-12 variant positioned at the hinge region, IL-12/PD-L2-Fc immunocytokine comprising IL-12 (G64A) variant showed the highest toxicity, as indicated by the death of 3/5 mice in low dose group and the death of 5/5 mice in high dose group. This is consistent with the highest toxicity results of IL-12 (G64A) among all IL-12 variants in Example 14. When placing IL-12 (F60A) variant at the C-terminus of the PD-L2-hinge-Fc polypeptide, 2 out of 5 mice died in high dose group. In contrast, when IL-12 (F60A) variant was positioned at the hinge region, all mice survived, even administered with high dose of immunocytokines (1000 μg). Consistent with results in Example 14, IL-12/PD-L2-Fc immunocytokine comprising IL-12 double mutation E59A/F60A demonstrated less toxicity compared to that comprising IL-12 single F60A mutation, no matter IL-12 variant was positioned at the hinge region or at the C-terminus of Fc, as indicated by the differences in severity of toxicity symptoms. Dose-dependent toxicity was observed for most immunocytokines, as indicated by increased severity of toxicity symptoms such as worse fur texture, increased weight loss, and/or greater reduced activity when dose was increased from 200 μg to 1000 μg. IL-12(E59A/F60A)/PD-L2-Fc immunocytokine comprising IL-12 variant positioned at the hinge region actually demonstrated the least toxicity in vivo among all IL-12/PD-L2-Fc and PD-L2-Fc/IL-12 immunocytokines, with 0 death rate and no toxicity symptom even when administered at high dose.

Comparing Table 14 and Table 15, our results indicate that replacing anti-PD-1 antigen-binding fragment with PD-L2 ligand in the IL-12-based immunocytokines can further reduce overall toxicity. For example, compare 3/5 death rate in low dose group of IL-12(G64A)/PD-L2-Fc immunocytokine vs. 4/5 death rate in low dose group of IL-12(G64A)/anti-PD-1 immunocytokine; compare 0 death rate in high dose group of IL-12(F60A)/PD-L2-Fc immunocytokine vs. 1/5 death rate in high dose group of IL-12(F60A)/anti-PD-1 immunocytokine. When comparing toxicity symptoms between the respective IL-12 variant immunocytokines in Example 14 and Example 15, the lower toxicity of PD-L2-Fc based immunocytokines is even more obvious. For example, IL-12(E59A/F60A)/PD-L2-Fc immunocytokine (construct #29) completely eliminated toxicity symptom compared to IL-12(E59A/F60A)/anti-PD-1 immunocytokine (construct #48), administered with either low or high dose; IL-12(F60A)/PD-L2-Fc immunocytokine (#30) showed fewer toxicity symptoms (fur texture only) compared to those of IL-12(F60A)/anti-PD-1 immunocytokine, either in low or high dose group. The reduced toxicity seen in PD-L2-Fc based IL-12 immunocytokines was likely due to stimulated PD-1 inhibitory immune checkpoint signaling upon PD-L2-PD-1 binding, which created an immunosuppression signal that “balances” against the immunostimulating/pro-inflammatory activity of IL-12.

TABLE 15
In vivo toxicity of IL-12/PD-L2-Fc immunocytokines

| | | Dose | | |
|---|---|---|---|---|
| Position | Construct | (μg) | Deaths | Toxicity Symptoms |
| Hinge | IL-12(F60A)/PD-L2-Fc | 200 | None | Fur texture (moderate) |
| | immunocytokine | 1000 | None | Fur texture |
| | (construct #30) | | | |
| | IL-12(G64A)/PD-L2-Fc | 200 | 3/5 | Fur texture, reduced activity, |
| | immunocytokine | | | weight loss, morbidity |
| | | 1000 | 5/5 | Fur texture, reduced activity, |
| | | | | weight loss, morbidity |
| | IL-12(E59A/F60A)/ | 200 | None | None |
| | PD-L2-Fc immunocytokine | 1000 | None | None |
| | (construct #29) | | | |
| C- | PD-L2-Fc/IL-12(F60A) | 200 | none | Fur texture, reduced activity, |
| terminus | immunocytokine | | | weight loss |
| | (construct #34) | 1000 | 2/5 | Fur texture, reduced activity, |
| | | | | weight loss, morbidity |
| | PD-L2-Fc/IL-12(E59A/ | 200 | None | Fur texture |
| | F60A) | 1000 | None | Fur texture, reduced activity, |
| | immunocytokine | | | weight loss |

### Example 16: In Vivo Efficacy of IL-12 Immunocytokines in 4T1 Syngeneic Tumor Mice Model

### Construction of IL-12(E59A/F60A)/IL-2(R38D/K43E/E61R)/Anti-PD-1 Immunocytokine

As described in Examples 3 and 4, an anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. The IL-2 R38D/K43E/E61R variant (SEQ ID NO: 2) was positioned within the hinge region of one heavy chain of the heterodimeric anti-PD-1 antibody, and the single-chain IL-12 E59A/F60A variant (SEQ ID NO: 36) was positioned within the hinge region of the other heavy chain of the heterodimeric anti-PD-1 antibody, to construct the “IL-12(E59A/F60A)/IL-2(R38D/K43E/E61R)/anti-PD-1 immunocytokine” (or “construct #54”). The immunocytokine was expressed and purified as described in Example 1.

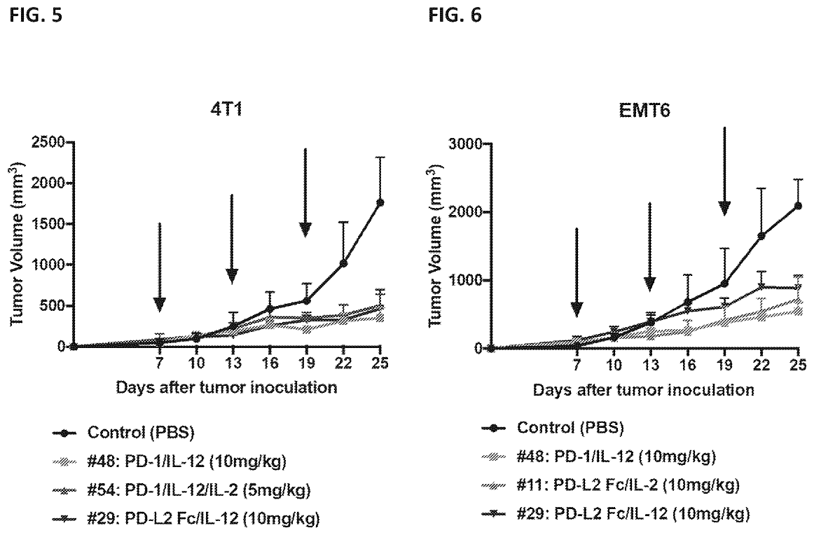

Mice (˜20 g body weight) were inoculated with 0.25×10⁶ 4T1 murine breast cancer cells. Seven days after tumor inoculation, tumor size was measured to be about 50-150 mm³. After measuring tumor size, mice were injected with 10 mg/kg (˜200 μg) IL-12(E59A/F60A)/anti-PD-1 immunocytokine (constructed in Examples 4 and 14; construct #48 in FIG. 5), 10 mg/kg (˜200 pg) IL-12(E59A/F60A)/PD-L2-Fc immunocytokine (constructed in Example 15; construct #29 in FIG. 5), 5 mg/kg (˜100 μg) IL-12(E59A/F60A)/IL-2(R38D/K43E/E61R)/anti-PD-1 immunocytokine (construct #54 in FIG. 5), or PBS (negative control). A total of three injections (10 mg/kg or 5 mg/kg per injection, respectively) were given on days 7, 13, and 19 post-inoculation (indicated by black arrows in FIG. 5). Tumor size was measured every 3 days since the first injection. Mice were sacrificed once tumor size reached over 2000 mm³.

Breast cancer as reflected by 4T1 mice model is highly resistant to current immunotherapies, including anti-PD-1, anti-CTLA-4, and combination treatment with anti-PD-1 and anti-CTLA-4 antibodies. As can be seen from FIG. 5, all three IL-12 immunocytokines significantly inhibited 4T1 tumor growth, demonstrating promising in vivo efficacy.

Further, IL-12(E59A/F60A)/anti-PD-1 (#48) and IL-12(E59A/F60A)/PD-L2-Fc (#29) showed similar cytotoxicity against 4T1 tumor when administered at the same dose (FIG. 5). In combination with results from Examples 14 and 15, these data further demonstrate that compared to anti-PD-1 antigen-binding domain which blocks PD-1 immunosuppression signal, using PD-L2 extracellular domain in the immunocytokine construct can not only achieve similar anti-tumor effect, but also reduce unwanted toxicity, likely by balancing the immunostimulating/pro-inflammatory activity of cytokines (e.g., IL-12) with an immunosuppression signal from PD-L2/PD-I signaling.

### Example 17: In Vivo Efficacy of IL-12 Immunocytokines and IL-2 Immunocytokines in EMT6 Syngeneic Tumor Mice Model

Mice (˜20 g body weight) were inoculated with 0.25×10⁶ EMT6 murine breast cancer cells. Seven days after tumor inoculation, tumor size was measured to be about 50-150 mm³. After measuring tumor size, mice were injected with 10 mg/kg (˜200 μg) IL-I2(E59A/F60A)/anti-PD-1 immunocytokine (constructed in Examples 4 and 14; construct #48 in FIG. 6), 10 mg/kg (˜200 pg) IL-12(E59A/F60A)/PD-L2-Fc immunocytokine (constructed in Example 15; construct #29 in FIG. 6), 10 mg/kg (˜200 μg) IL-2(R38D/K43E/E61R)/PD-L2-Fc immunocytokine (constructed in Example 3; construct #11 in FIG. 6), or PBS (negative control). A total of three injections (10 mg/kg per injection) were given on days 7, 13, and 19 post-inoculation (indicated by black arrows in FIG. 6). Tumor size was measured every 3 days since the first injection. Mice were sacrificed once tumor size reached over 2000 mm³.

EMT6 tumor growth is resistant to anti-PD-1 immunotherapy. As can be seen from FIG. 6, all immunocytokine significantly inhibited EMT6 tumor growth, of which IL-12(E59A/F60A)/anti-PD-1 and IL-2(R38D/K43E/E61R)/PD-L2-Fc immunocytokines demonstrated better efficacy compared to IL-12(E59A/F60A)/PD-L2-Fc immunocytokine. The slightly lower efficacy seen in PD-L2-Fc based IL-12 immunocytokine was likely due to stimulated PD-1 inhibitory immune checkpoint signaling upon PD-L2-PD-1 binding, which created an immunosuppression signal that “balances” against the immunostimulating/pro-inflammatory activity of IL-12.

### Example 18: In Vivo Efficacy of IL-12 Immunocytokines in CT26 Syngeneic Tumor Mice Model

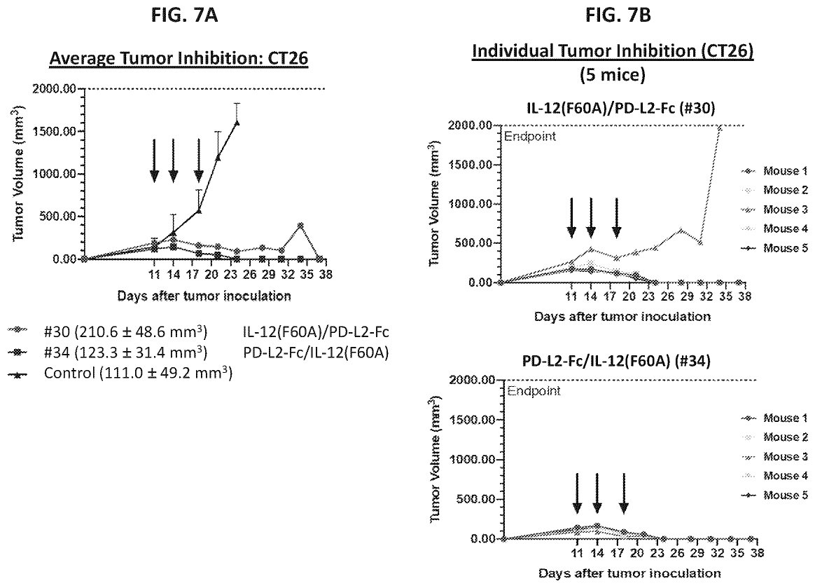

Mice (˜20 g body weight) were inoculated with 0.25×10⁶ CT26 murine colon cancer cells. When tumor size reached about 100-200 mm³ (about day 11 post-inoculation), mice were injected with 10 mg/kg IL-12(F60A)/PD-L2-Fc immunocytokine (constructed in Example 15; construct #30; 5 mice), 10 mg/kg PD-L2-Fc/IL-12(F60A) immunocytokine (constructed in Example 15; construct #34; 5 mice), or PBS (negative control). A total of three injections (10 mg/kg per injection) were given on days 11, 14, and 18 post-inoculation (indicated by black arrows in FIGS. 7A and 7B). Tumor size was recorded over time. Mice were sacrificed once tumor size reached over 2000 mm³. Tumor size in parenthesis of FIG. 7A indicates average tumor size (±standard deviation) of each group when the first treatment was administered.

As can be seen in FIGS. 7A and 7B, PD-L2-Fc/IL-12(F60A) immunocytokine (#34) successfully inhibited tumor growth in all five mice (100% cure rate), and IL-12(F60A)/PD-L2-Fc immunocytokine (#30) successfully inhibited tumor growth in four out of five mice (80% cure rate). Among cured mice, the tumor inhibition efficacy of both IL-12 immunocytokines was similar, suggesting no significant efficacy difference between IL-12 hinge fusion and C-terminus fusion. This result shows both IL-12 immunocytokines tested can completely regress CT26 syngeneic colon tumors in mice.

### Example 19: In Vivo Efficacy of IL-12 Immunocytokines as a Cancer Vaccine Against CT26 Tumors

To investigate if immunocytokines described herein can function as cancer vaccine, or prevent cancer recurrence, a tumor re-challenge was conducted on all cured mice from Example 18. Thirty days after the final immunocytokine injection, cured mice were inoculated with 0.25×10⁶ CT26 murine colon cancer cells on the right flank and 0.25×10⁶ EMT6 murine breast cancer cells on the left flank (as control). Tumor sizes were recorded every 4 days following re-challenge tumor inoculation.

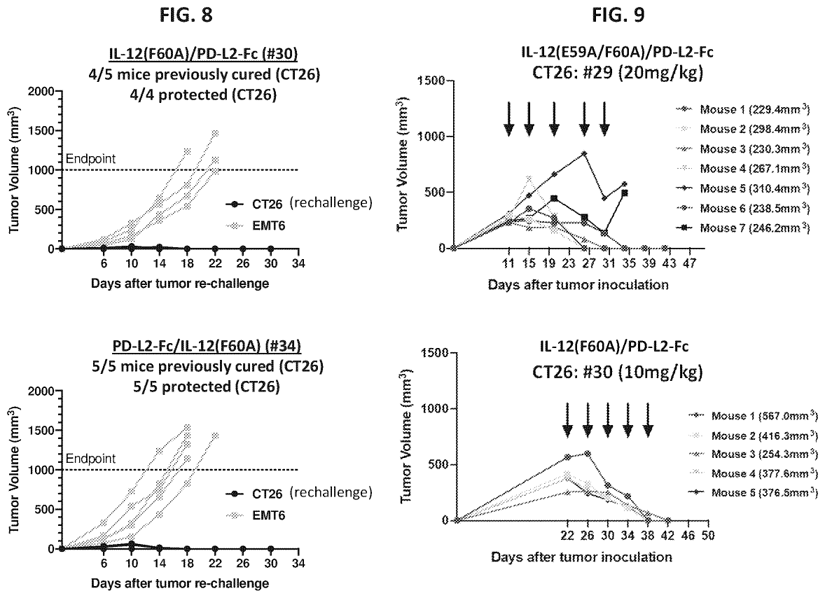

As shown in FIG. 8, all cured mice previously treated with immunocytokines against CD26 tumor were protected from CT26 tumor re-challenge but not from EMT6. IL-12(F60A)/PD-L2-Fc immunocytokine (#30) and PD-L2-Fc/IL-12(F60A) immunocytokine (#34) demonstrated similar protection efficacy against CT26 tumor re-challenge. These results indicate successful generation of anti-CT26 tumor memory, suggesting that immunocytokines described herein, such as both PD-L2-Fc/IL-12(F60A) immunocytokine (#34) and IL-12(F60A)/PD-L2-Fc immunocytokine (#30), can serve as a cancer vaccine (e.g., against CT26 colon cancer) in mice, and/or can prevent cancer recurrence.

### Example 20: In Vivo Efficacy of IL-12 Immunocytokines in Late-Stage CT26 Syngeneic Tumor Mice Model

Successful therapies for late-stage cancers remain a huge unmet clinical need. To study if immunocytokines described herein are effective in treating late-stage cancers, mice were inoculated with cancer cells, tumor was allowed to grow to bigger than 250 mm³, which is considered untreatable with immunotherapy in mice. Such murine tumor volume may mimic tumor burdens in advanced, late-stage human cancer patients.

Briefly, mice (˜20 g body weight) were inoculated with 0.25×10⁶ CT26 murine colon cancer cells. When tumor size reached about 250-400 mm³, mice were injected with 20 mg/kg IL-12(E59A/F60A)/PD-L2 Fc immunocytokine (constructed in Example 15; construct #29; 7 mice) or 10 mg/kg IL-12(F60A)/PD-L2-Fc immunocytokine (constructed in Example 15; construct #30; 5 mice). A total of five injections were given on days 11, 15, 20, 26, and 30 post-inoculation for mice treated with construct #29, or on days 22, 26, 30, 34, and 38 post-inoculation for mice treated with construct #30 (indicated by black arrows in FIG. 9). Tumor size in parenthesis of FIG. 9 indicates tumor size of each mouse when the first treatment was administered, which was similar to or bigger than 250 mm³. Tumor size was recorded over time.

As shown in FIG. 9, all mice receiving IL-12(F60A)/PD-L2-Fc immunocytokine (#30) had significantly reduced tumor size (100% cure rate); while 5/7 (71.4% cure rate) mice receiving IL-12(E59A/F60A)/PD-L2 Fc immunocytokine (#29) had significantly reduced tumor size. These indicate that both IL-12 immunocytokines are effective in treating late-stage cancer (e.g., late stage colon cancer). The efficacy difference seen was due to lower potency (e.g., receptor binding and/or signal activation ability) of double mutation IL-12(E59A/F60A) compared to single mutation IL-12(F60A). Such efficacy difference may be compensated by higher dosing per injection (e.g., 20 mg/kg vs. 10 mg/kg), or more injections (e.g., increase from 3 to 5 injections) of IL-12(E59A/F60A)-based immunocytokines.

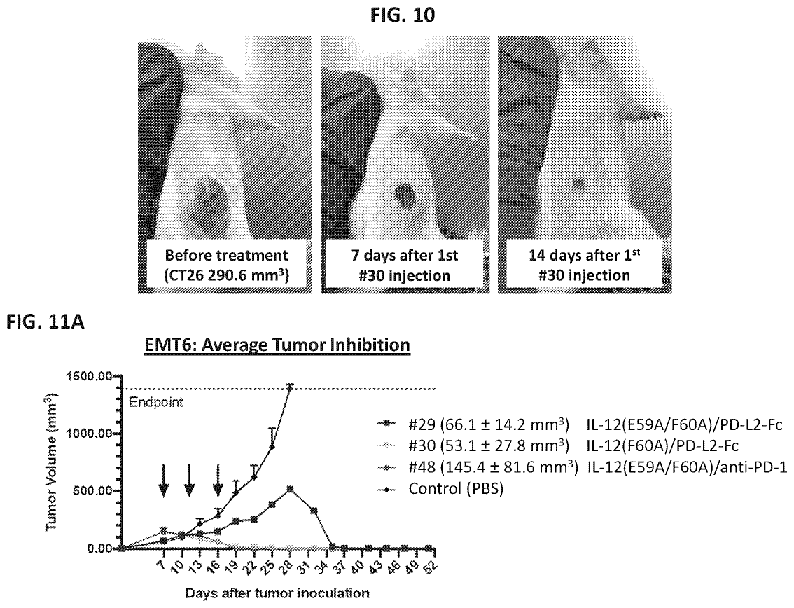

FIG. 10 shows one exemplary mouse inoculated with CT26 tumor, the initial tumor volume was measured to be 290.6 mm³. Pictures were taken on 0, 7, and 14 days after initial injection of IL-12(F60A)/PD-L2-Fc immunocytokine (#30), indicating successful regression of huge tumor (equivalent to advanced, late-stage human cancer).

### Example 21: In Vivo Efficacy of IL-12 Immunocytokines in EMT6 Syngeneic Tumor Mice Model

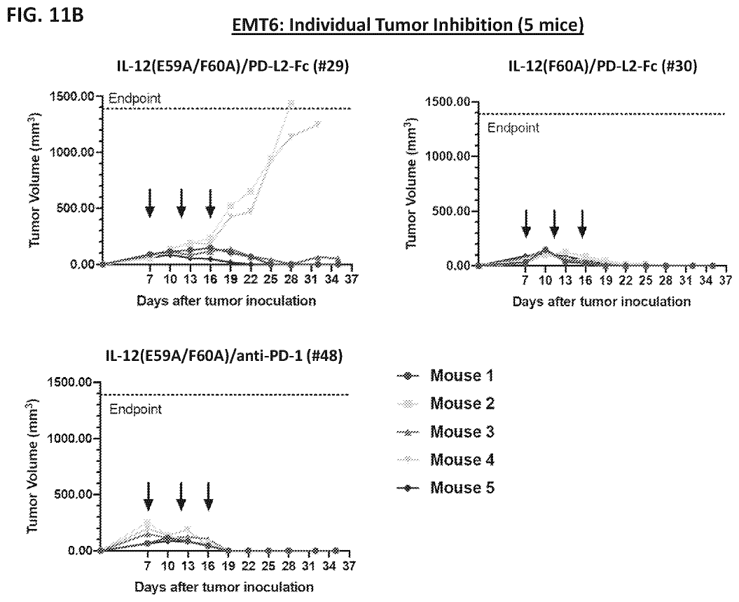

Mice (˜20 g body weight) were inoculated with 0.25×10⁶ EMT6 murine breast cancer cells. When tumor size reached about 50-100 mm³ (˜7 days after tumor inoculation), mice were injected with 10 mg/kg IL-12(E59A/F60A)/PD-L2 Fc immunocytokine (constructed in Example 15; construct #29), 10 mg/kg IL-12(F60A)/PD-L2-Fc immunocytokine (constructed in Example 15; construct #30), 10 mg/kg IL-12(E59A/F60A)/anti-PD-1 immunocytokine (constructed in Example 4; construct #48), or PBS (negative control). A total of three injections (10 mg/kg per injection) were given on days 7, 12, and 16 post-innoculation (indicated by black arrows in FIGS. 11A and 11B). Tumor size was recorded over time. Mice were sacrificed once tumor size reached over 1500 mm³. Tumor size in parenthesis of FIG. 11A indicates average tumor size (±standard deviation) of each group when the first treatment was administered.

As seen in FIGS. 11A and 11B, IL-12(E59A/F60A)/PD-L2 Fc immunocytokine (#29) successfully inhibited tumor growth in 3/5 mice (60% cure rate); IL-12(F60A)/PD-L2-Fc immunocytokine (#30) and IL-12(E59A/F60A)/anti-PD-1 immunocytokine (#48) both successfully inhibited tumor growth in all five mice (100% cure rate). The initial average tumor size of the mouse group treated with IL-12(E59A/F60A)/anti-PD-1 immunocytokine (#48) was more than twice of that of the other two test groups. These results show all three IL-12 immunocytokines tested can completely regress EMT6 syngeneic breast tumors in mice, with anti-PD-1 based immunocytokine having the best efficacy.

### Example 22: In Vivo Efficacy of IL-12 Immunocytokines as a Cancer Vaccine Against EMT6 Tumors

To investigate if immunocytokines described herein can function as cancer vaccine, or prevent cancer recurrence, a tumor re-challenge was conducted on all cured mice from Example 21. Thirty days after the final immunocytokine injection, cured mice were inoculated with 0.25×10⁶ EMT6 murine breast cancer cells on the right flank and 0.25×10⁶ CT26 murine colon cancer cells on the left flank (as control). Tumor sizes were recorded every 4 days following re-challenge tumor inoculation.

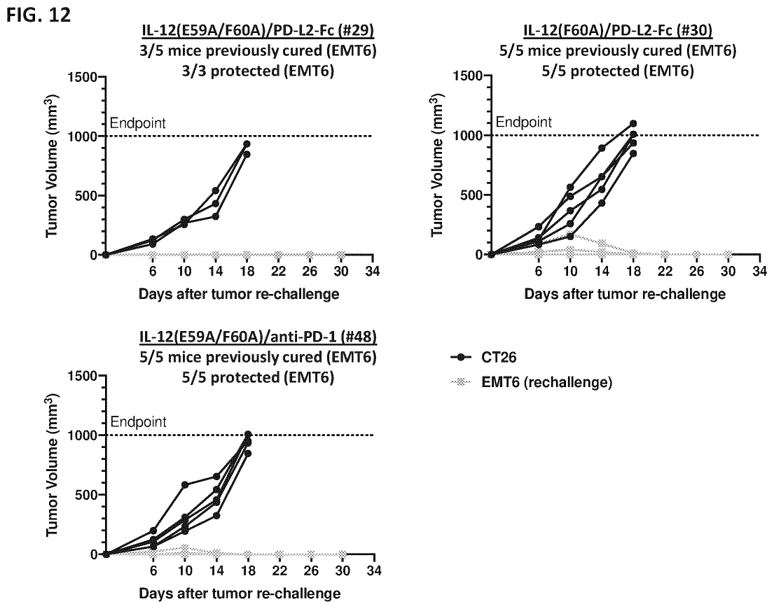

As seen in FIG. 12, all cured mice previously treated with immunocytokines against EMT6 tumor were protected from EMT6 tumor re-challenge but not from CT26. Further, all three IL-12 immunocytokines demonstrated similar protection efficacy against EMT6 tumor re-challenge. These results indicate successful generation of anti-EMT6 tumor memory, suggesting that immunocytokines described herein, such as IL-12(E59A/F60A)/PD-L2 Fc immunocytokine (#29), IL-12(F60A)/PD-L2-Fc immunocytokine (#30), and IL-12(E59A/F60A)/anti-PD-1 immunocytokine (#48), can serve as a cancer vaccine (e.g., against breast cancer (such as EMT6) tumors) in mice, and/or can prevent cancer recurrence.

### Example 23: In Vivo Efficacy of IL-12 Immunocytokines in 4T1 Triple Negative Breast Cancer (TNBC) Orthotopic Tumor Mice Model

4T1 is a standard murine mammary tumor model used in preclinical studies on breast cancer metastasis. 4T1 is a refractory model for immunotherapy and does not respond to anti-PD-1, anti-CTLA-4, or combination of anti-PD-1 and anti-CTLA-4 antibody therapy. Mammary fat pad injection of 4T1 can reproducibly generate 4T1 breast-cancer-derived lung metastases.

To test the therapeutic efficacy of immunocytokines described herein on immunotherapy-resistant cancer types as well as cancer metastasis, mice (˜20 g body weight) were inoculated with 0.25×10⁶ 4T1 murine breast cancer cells in the #3 mammary gland fat pad. Tumor development was monitored for approximately 21-30 days. Four days after tumor inoculation, mice were injected with 20 mg/kg (per injection) IL-12(E59A/F60A)/PD-L2-Fc immunocytokine (constructed in Example 15; construct #29), 20 mg/kg (per injection) IL-12(F60A)/PD-L2-Fc immunocytokine (constructed in Example 15; construct #30), a combination of 10 mg/kg anti-PD-1 antibody and 10 mg/kg anti-CTLA-4 antibody (per injection), or PBS (negative control). A total of five injections were given every four days. Mice were sacrificed after four weeks and primary tumor was extracted from the mammary fat pad.

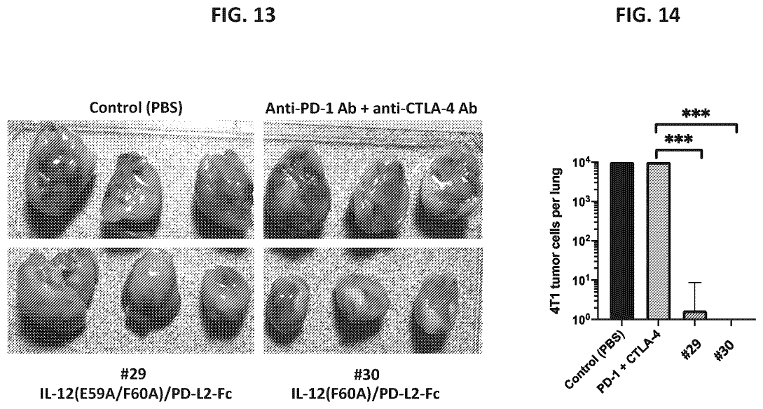

As seen in FIG. 13, compared to PBS control, IL-12(F60A)/PD-L2-Fc immunocytokine (#30) inhibited 4T1 growth in mammary gland in all mice tested, IL-12(E59A/F60A)/PD-L2-Fc immunocytokine (#29) inhibited 4T1 growth in mammary gland in 1 out of 3 mice tested, while anti-PD-1+anti-CTLA-4 combination treatment failed inhibiting 4T1 growth in mammary gland in all 3 mice tested. As discussed above, the efficacy difference was likely due to lower potency (e.g., receptor binding and/or signal activation ability) of double mutation IL-12(E59A/F60A) compared to single mutation IL-12(F60A). Such efficacy difference may be compensated by higher dosing per injection (e.g., 40 mg/kg vs. 20 mg/kg), or more injections (e.g., increase from 5 to 7 injections) of IL-12(E59A/F60A)-based immunocytokines.

To investigate the therapeutic efficacy on cancer metastasis, lungs were retrieved from sacrificed mice. Lung tissue was resuspended in collagenase/DNase solution and filtered through a 70 μm cell strainer. Cells were washed with PBS and resuspended in media. Four 1:10 serial dilutions were made. Cells were cultured in a 7% C02 incubator at 37° C. for 14 days to allow the formation of 4T1 cell colonies.

As seen in FIG. 14, IL-12(E59A/F60A)/PD-L2-Fc immunocytokine (#29) and IL-12(F60A)/PD-L2-Fc immunocytokine (#30) both significantly inhibited 4T1 metastasis in the lungs compared to the combination of anti-PD-1 and anti-CTLA-4 antibodies, or PBS (negative control). These findings were statistically significantly different (p-value≤0.001).

These data demonstrating promising in vivo efficacy of IL-12 immunocytokines in treating advanced and/or hard-to-treat breast cancer (e.g., TNBC), inhibiting cancer metastasis, and possibly in treating other cancer types that are resistant to current immunotherapies.

### Example 24: In Vivo Efficacy of IL-12 Immunocytokines in B16 Syngeneic Tumor Mice Model

B16 a murine melanoma tumor cell line used for research as a model for human skin cancers. B16 is a refractory model for immunotherapy and does not respond to anti-PD-1, anti-CTLA-4, or combination of anti-PD-1 and anti-CTLA-4 antibody therapy.

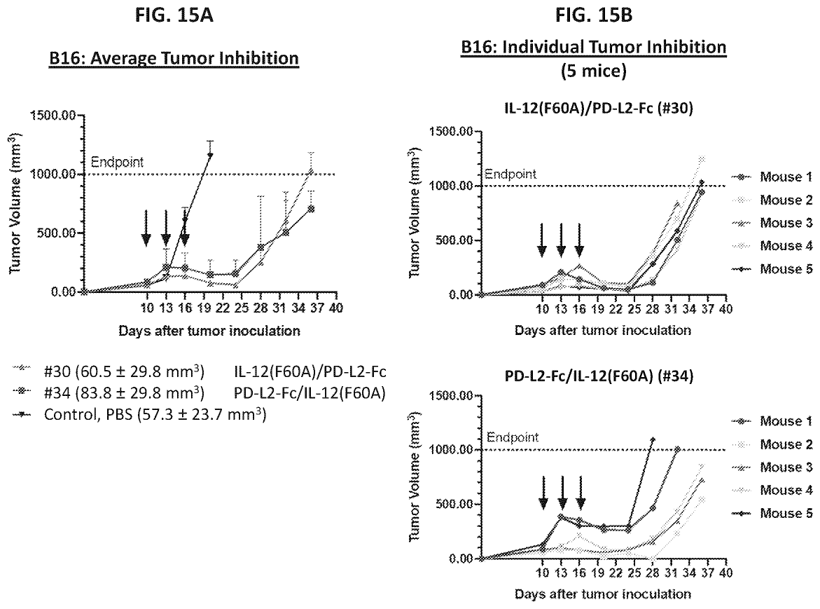

To test the therapeutic efficacy of immunocytokines described herein on more immunotherapy-resistant cancer types, mice (˜20 g body weight) were inoculated with 0.25×10⁶ B16 murine melanoma cells. When tumor size reached about 50-100 mm³, mice were injected with 10 mg/kg IL-12(F60A)/PD-L2-Fc immunocytokine (constructed in Example 15; construct #30), 10 mg/kg PD-L2-Fc/IL-12(F60A) immunocytokine (constructed in Example 15, construct #34), or PBS (negative control). A total of three injections (10 mg/kg per injection) were given on days 10, 13, and 16 post-innoculation (indicated by black arrows in FIGS. 15A and 15B). Tumor size was recorded over time. Mice were sacrificed once tumor size reached over 1000 mm³. Tumor size in parenthesis of FIG. 15A indicates average tumor size (±standard deviation) of each group when the first treatment was administered.

As seen in FIGS. 15A and 15B, compared to PBS treatment group in which B16 tumor grew drastically since day 13 post-innoculation, PD-L2-Fc/IL-12(F60A) immunocytokine (#34) and IL-12(F60A)/PD-L2-Fc immunocytokine (#30) both significantly inhibited B16 tumor growth until after day 24 post-innoculation, indicating that both IL-12 immunocytokines can slow down tumor progression and/or extend life-span of individuals with immunotherapy-resistant cancers (e.g., melanoma), demonstrating promising in vivo efficacy.

### Example 25: In Vivo Efficacy of IL-12 Immunocytokines in LL2 Syngeneic Tumor Mice Model

LL2 murine lung carcinoma model is a refractory model for immunotherapy that does not respond to anti-PD-1, anti-CTLA-4, or combination of anti-PD-1 and anti-CTLA-4 antibody therapy.

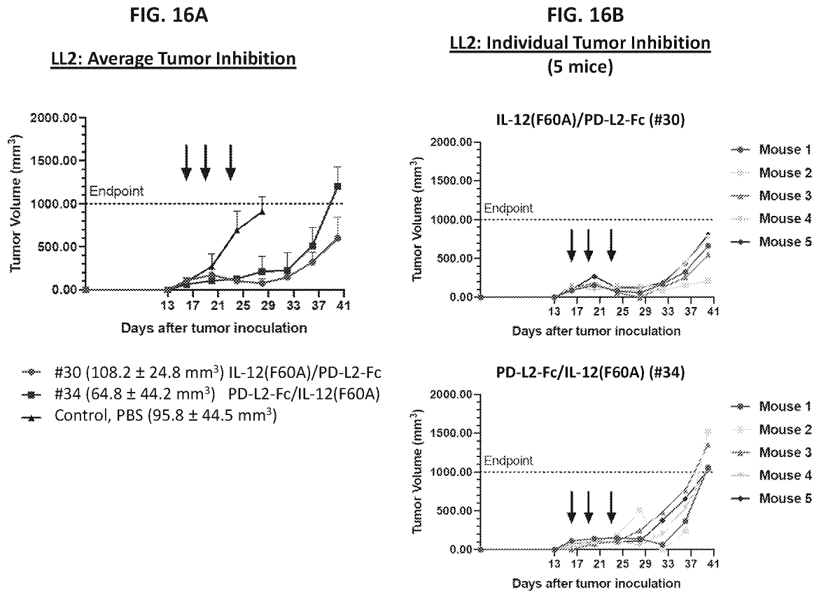

To test the therapeutic efficacy of immunocytokines described herein on more immunotherapy-resistant cancer types, mice (˜20 g body weight) were inoculated with 0.25×10⁶ LL2 murine lung cancer cells. When tumor size reached about 50-100 mm³ (˜16 days post-inoculation), mice were injected with 10 mg/kg IL-12(F60A)/PD-L2-Fc immunocytokine (constructed in Example 15; construct #30), 10 mg/kg PD-L2-Fc/IL-12(F60A) immunocytokine (constructed in Example 15, construct #34), or PBS (negative control). A total of three injections (10 mg/kg per injection) were given on days 16, 20, and 23 post-innoculation (indicated by black arrows in FIGS. 16A and 16B). Tumor size was recorded over time. Mice were sacrificed once tumor size reached over 1000 mm³. Tumor size in parenthesis of FIG. 16A indicates average tumor size (±standard deviation) of each group when the first treatment was administered.

As seen in FIGS. 16A and 16B, compared to PBS treatment group in which LL2 tumor grew drastically since about day 20 post-innoculation, PD-L2-Fc/IL-12(F60A) immunocytokine (#34) and IL-12(F60A)/PD-L2-Fc immunocytokine (#30) significantly inhibited LL2 tumor growth until after day 32-35 post-innoculation, indicating that both IL-12 immunocytokines can slow down tumor progression and/or extend life-span of individuals with immunotherapy-resistant cancers (e.g., lung cancer), demonstrating promising in vivo efficacy.

To summarize, above data (e.g., see Examples 23-25) demonstrate promising in vivo efficacy of immunocytokines described herein (e.g., IL-12/PD-L2-Fc based immunocytokines) in treating various advanced and/or hard-to-treat cancer types (e.g., TNBC, melanoma, lung cancer), inhibiting cancer metastasis, treating or delaying tumor progression of cancer types that are resistant to current immunotherapies (e.g., anti-PD-1 therapy, anti-CTLA-4 therapy, or a combination therapy thereof), and/or extending life-span of such patients.

### Example 26: Position of Cytokine or Variant Thereof within the Immunocytokine Affects Non-Specific Activities of the Immunocytokine

Two immunocytokine designs were generated to test whether placement of the cytokine or variant thereof at the hinge region (between antigen-binding domain and Fc fragment; hidden format) or at the C-terminus of the Fc fragment (e.g., C′ of antibody heavy chain; exposed format) could affect the targeted activity of the cytokine or variant thereof. The first design incorporated the cytokine at the hinge region of one heavy chain of an anti-PD-1 antibody: within the hinge region between CH1 and CH2 (the immunocytokines were named in the format of “IL-12/anti-PD-1”). The second design fused the cytokine to the C-terminus of one heavy chain of an anti-PD-1 antibody through a linker (the immunocytokines were named in the format of “anti-PD-1/IL-12”), which is a common design among current immunocytokines.

An anti-human PD-1 antibody comprising nivolumab (Opdivo®) VH (SEQ ID NO: 102) and VL (SEQ ID NO: 103) sequences was used as the parental full-length antibody, comprising two light chains each comprising the amino acid sequence of SEQ ID NO: 106. To construct heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62.

IL-12(E59A/F60A)/anti-PD-1 immunocytokine (construct #48), IL-12(E59A)/anti-PD-1 immunocytokine (construct #46), and IL-12(G64A)/anti-PD-1 immunocytokine (construct #47) with IL-12 variant (single-chain N′ to C′ IL-12B (p40 variant)-linker-IL-12A (wt p35)) positioned within the hinge region of one heavy chain of the heterodimeric anti-PD-1 antibody were constructed as described in Examples 4 and 14.

To make the heavy chain C′ cytokine fusion constructs, single-chain IL-12(E59A) variant (SEQ ID NO: 331), single-chain IL-12(G64A) variant (SEQ ID NO: 332), or single-chain IL-12(E59A/F60A) variant (SEQ ID NO: 36) was fused to the C′ of one heavy chain of the heterodimeric anti-PD-1 antibody via a G/S containing peptide linker. The constructs are hereinafter referred to as anti-PD-1/IL-12(E59A) (construct #46HC′; heavy chain cytokine fusion polypeptide, heavy chain non-fusion polypeptide SEQ ID NO: 107), anti-PD-1/IL-12(G64A) (construct #47HC′; heavy chain cytokine fusion polypeptide, heavy chain non-fusion polypeptide SEQ ID NO: 107), and anti-PD-1/IL-12(E59A/F60A) (construct #48HC′; heavy chain cytokine fusion polypeptide, heavy chain non-fusion polypeptide SEQ ID NO: 107), respectively.

IL-12 signal transduction assays using HEK-Blue™ IL-12 and HEK-PD-I-IL-12 (generated in-house by overexpressing human PD-1 in HEK-Blue™ IL-12 Cells using a lentiviral vector) cells were similarly conducted as described in Example 4 with two configurations of immunocytokines described above and rIL-12 (positive control).

TABLE 16
IL-12 biological activity of different IL-12 immunocytokine formats

| | Location of | IL-12 mutation | | HEK-PD-1- |
|---|---|---|---|---|
| Construct | cytokine | (in p40 subunit) | HEK-IL-12 | IL-12 |
| rIL12 (control) | / | / | 100% | 100% |
| IL-12(G64A)/anti-PD-1 | Hinge of | G64A | 90% | 230% |
| (construct #47) | one heavy | | | |
| IL-12(E59A)/anti-PD-1 | chain | E59A | 5% | 78% |
| (construct #46) | | | | |
| IL-12(E59A/F60A)/anti-PD-1 | | E59A/ | <0.2% | 32% |
| (construct #48) | | F60A | | |
| anti-PD-1/IL-12(G64A) | C-terminus | G64A | 85% | 210% |
| (construct #47HC′) | of one | | | |
| anti-PD-1/IL-12(E59A) | heavy chain | E59A | 15% | 86% |
| (construct #46HC′) | | | | |
| anti-PD-1/IL-12(E59A/F60A) | | E59A/ | 4% | 37% |
| (construct #48HC′) | | F60A | | |

In HEK-IL-12 reporter assay, both IL-12 immunocytokine formats were only able to bind to HEK-IL-12 cells via IL-12 moiety/IL-12 receptor interaction, if the IL-12 moiety was accessible (e.g., heavy chain C′ fusion format). In HEK-PD-1-IL-12 reporter assay, both IL-12 immunocytokine formats were able to bind to HEK-PD-I-IL-12 cells via both IL-12 moiety/IL-12 receptor interaction, and anti-PD-1 antigen-binding fragment/PD-1 interaction.

As shown in Table 16, the hinge fusion design had significantly decreased non-specific activity (i.e., cytokine activity in the absence of PD-1 binding) compared to the heavy chain C-terminus fusion design. In PD-1 negative cells (HEK-IL-12), construct #48 showed almost undetectable levels of IL-12 activity (≤0.2%), compared to 4% for construct #48HC′. Similar results were observed for construct #47 and construct #47HC′ (5% compared to 15%, respectively). The IL-12 double mutation E59A/F60A also significantly reduced non-specific activity compared to single mutation E59A or G64A. In PD-1 positive cells (HEK-PD-1-IL-12), IL-12 targeted activity was similar between the corresponding hinge fusion format and heavy chain C-terminus fusion format, suggesting that the hinge fusion design does not significantly inhibit IL-12 activity in the presence of antigen-positive cells (or antigen-binding). Taken together, the hinge placement of cytokine (especially certain cytokine variants) can greatly reduce non-specific IL-12 activity in the absence of binding of the antigen-binding domain.

### Example 27: Generation of IL-12/Anti-PD-1 Immunocytokines with Reduced Affinity for PD-1

Generation of Anti-PD-1 Antibody Variants with Reduced PD-1 Binding Affinity

Due to nivolumab's high binding affinity for PD-1, IL-12/anti-PD-1 immunocytokines using wildtype nivolumab as parental antibody may direct IL-12 activity to all PD-1 positive cells, regardless of PD-1 expression levels. Activation of such a large population of PD-1 positive cells could result in a cytokine storm or other adverse side effects, from activating any PD-I positive immune cells.

To generate anti-PD-1 mutants with reduced binding affinity for PD-1, so that it only targets high expressing PD-1 cells (e.g., T cells), mutations were introduced to HC-CDR3 at D100 or N99 positions of nivolumab: HC-CDR3(D100N), HC-CDR3(D100G), HC-CDR3(D100R), HC-CDR3(N99G), HC-CDR3(N99A) and HC-CDR3(N99M). The affinity of these anti-PD-1 antibodies were measured by Biacore and cell-based assays, calibrated by wildtype nivolumab binding affinity (see Table 17). “N/A” indicates non-detected PD-1 binding.

To construct anti-PD-1 heterodimer, one heavy chain comprises a hinge region comprising SEQ ID NO: 42, and an Fc domain subunit comprising SEQ ID NO: 61; the other heavy chain comprises a hinge region comprising SEQ ID NO: 41, and an Fc domain subunit comprising SEQ ID NO: 62. The two light chains each comprises the amino acid sequence of SEQ ID NO: 106.

TABLE 17
PD-1 binding affinities of various anti-PD-1 heavy chain mutants

| Heavy chain mutation | Affinity to PD-1 (Kd) | |
|---|---|---|
| WT | 2.6 | nM |
| D100N | 25 | nM |
| D100G | 130 | nM |
| D100R | 910 | nM |
| N99G | 2300 | nM |

| | |
|---|---|
| N99A | N/A |
| N99M | N/A |

Construction of IL-12/Anti-PD-1 Immunocytokines with Reduced Affinity for PD-1

Various IL-12/anti-PD-1 immunocytokines were generated as described in Example 4 by placing single-chain IL-12(E59A/F60A) variant (SEQ ID NO: 36) within the hinge region of one heavy chain of the various anti-PD-1 mutants described above. The sequence of the heavy chain cytokine fusion polypeptide is provided in Table 18 for each construct. The corresponding pairing non-fusion heavy chain comprises from N′ to C′ VH (with corresponding HC-CDR3 mutation) —CH1—hinge (SEQ ID NO: 41)—Fc domain subunit (SEQ ID NO: 62).

### IL-12 Signal Transduction Assay

IL-12 signal transduction assays were similarly conducted as described in Example 4 using IL-12/anti-PD-1(mut) immunocytokines with reduced PD-1 binding affinity (IL-12(E59A/F60A)/anti-PD-1(wt) and rIL-12 served as control), on HEK-Blue™ IL-12 cells and HEK-PD-1-IL-12 cells. Two variations of HEK-PD-1-IL-12 cells were used: one with high PD-1 expression “HEK-IL-12-PD-1(high)” (as described in Example 4, over-expressing PD-1), and one with 30-fold lower PD-1 expression “HEK-IL-12-PD-1(low)” (generated in-house by expressing lower amount of human PD-1 in HEK-Blue™ IL-12 Cells using a lentiviral vector). Cells were incubated with 20 ng/mL of the various IL-12/anti-PD-1 immunocytokines (or control) for 24 hours.

### IFN-γ Release Assay

IFN-γ release assays were similarly conducted as described in Example 5 using the IL-12(E59A/F60A)/anti-PD-1(mut) immunocytokines with reduced PD-1 binding affinity. IL-12(E59A/F60A)/anti-PD-1(wt) and rIL-12 served as control. Briefly, T cells were activated by incubating PBMCs with an anti-CD3 antibody (OKT3, 100 ng/mL) for three days. PBMCs were washed to remove the anti-CD3 antibody and incubated with 200 ng/mL of the various IL-12(E59A/F60A)/anti-PD-1(mut) immunocytokines (or control) for 24 hours. After one day, the amount of IFN-γ released into the cell culture medium was measured.

TABLE 18
IL-12 biological activity of IL-12(E59A/F60A)/anti-PD-1 immunocytokines comprising
various anti-PD-1 heavy chain mutations (reduced PD-1 binding affinity)

| | Affinity | | | | | |
|---|---|---|---|---|---|---|
| Construct | PD-1 | | HEK- | HEK-IL-12- | HEK-IL-12- | PBMC |
| (heavy chain fusion sequence) | (Kd) | | IL-12 | PD-1 (high) | PD-1 (low) | (IFN-γ ng/ml) |
| rIL-12 | | | 100% | 100% | 100% | 2300 |
| IL-12(E59A/F60A)/anti-PD-1(WT) | 2.6 | nM | <0.2% | 36% | 38% | 1400 |
| (construct #48; SEQ ID NO: 112) | | | | | | |
| IL-12(E59A/F60A)/anti-PD- | 25 | nM | <0.2% | 38% | 33% | 820 |
| 1(D100N) | | | | | | |
| (SEQ ID NO: 268) | | | | | | |
| IL-12(E59A/F60A)/anti-PD- | 130 | nM | <0.2% | 33% | 17% | 120 |
| 1(D100G) | | | | | | |
| (SEQ ID NO: 269) | | | | | | |
| IL-12(E59A/F60A)/anti-PD- | 910 | nM | <0.2% | 10% | 3% | 69 |
| 1(D100R) | | | | | | |
| (SEQ ID NO: 270) | | | | | | |
| IL-12(E59A/F60A)/anti-PD- | 2300 | nM | <0.2% | 5% | <0.2% | 34 |

| | | | | | |
|---|---|---|---|---|---|
| 1(N99G) | | | | | |
| (SEQ ID NO: 271) | | | | | |
| IL-12(E59A/F60A)/anti-PD- | N/A | <0.2% | 3% | <0.2% | 12 |
| 1(N99A) | | | | | |
| (SEQ ID NO: 272) | | | | | |
| IL-12(E59A/F60A)/anti-PD- | N/A | <0.2% | <0.2% | <0.2% | 13 |
| 1(N99M) | | | | | |
| (SEQ ID NO: 273) | | | | | |

As can be seen from Table 18, for all immunotyokines tested, no non-specific IL-12 activity was observed in the absence of anti-PD-1 binding (see HEK-IL-12 column). Their ability of transducing IL-12 signal in the presence of PD-1 binding, as well as their ability in inducing IFN-γ release, decreases as anti-PD-1 binding affinity decreases, demonstrating antigen-binding dependent cytokine activity of the hinge fusion design. IL-12(E59A/F60A)/anti-PD-1 immunocytokines with the D100G, D100R, or N99G mutations in anti-PD-1 heavy chain showed notable differences in binding between high and low PD-1 expressing cells. These results indicate that cells expressing a higher level of PD-1 can be specifically targeted using IL-12(E59A/F60A)/anti-PD-1(mut) immunocytokines with reduced affinity for PD-1. IFN-γ secretion induced by these constructs were also much lower compared to the IL-12/anti-PD-1(wt) immunotyokine and rIL-12 control.

Hence IL-12(E59A/F60A)/anti-PD-1(mut) immunocytokines described herein, and maybe other immunocytokines constructed based on antigen-binding domain with reduced antigen binding affinity, may be used to specifically target cells of interest with high-antigen expression, with reduced off-target effect and/or cytokine storm.

### Example 28: Reducing PD-1 Binding Affinity in IL-12/Anti-PD-1 Immunocytokines Reduces Toxicity in Mice

Humanized PD-1 mice (by inserting, within the mouse PD-1 locus, a chimeric PD-1 with a human extracellular domain, a murine transmembrane domain and a murine intracellular domain) derived from the C57 strain (5-6 weeks age, 20 g females) were injected with 10 mg/kg or 50 mg/kg (per injection) of various IL-12(E59A/F60A)/anti-PD-1(mut) immunocytokines described in Example 27. IL-12(E59A/F60A)/anti-PD-1(wt) immunocytokine (construct #48) served as control. A total of four injections were given on Days 0, 4, 8, and 12. Mice were monitored daily for mortality and four toxicity symptoms: i) fur texture, ii) reduced activity, iii) morbidity, and iv) weight loss.

Mice injected with the IL-12(E59A/F60A)/anti-PD-1(wt) immunocytokine comprising wildtype nivolumab showed the greatest toxicity, with all mice in the group dying after receiving either the second or third injection, even for lower dosing. In contrast, mice injected with IL-12(E59A/F60A)/anti-PD-1(mut) immunocytokines comprising anti-PD-1 with reduced PD-1 binding affinity showed reduced toxicity, with death observed only in the group treated with IL-12(E59A/F60A)/anti-PD-1(D100N). As can be seen from Table 19, the severity of toxicity symptom reduces as PD-I binding affinity decreases, and/or as the dose decrease, among the constructs.

TABLE 19
In vivo toxicity of IL-12/anti-PD-1 immunocytokines

| | Affinity | | | | |
|---|---|---|---|---|---|
| | PD-1 | | Dose | | |
| Construct | (Kd) | | (mg/kg) | Toxicity Symptoms | Deaths |
| IL-12(E59A/F60A)/ | 2.6 | nM | 10 | Fur texture, reduced activity, | 5/5 |
| anti-PD-1(WT) | | | | weight loss, morbidity | |
| (construct #48) | | | 50 | Fur texture, reduced activity, | 5/5 |
| | | | | weight loss, morbidity | |
| IL-12(E59A/F60A)/ | 25 | nM | 10 | Fur texture, reduced activity, | 3/5 |
| anti-PD-1(D100N) | | | | weight loss, morbidity | |
| | | | 50 | Fur texture, reduced activity, | 5/5 |
| | | | | weight loss, morbidity | |
| IL-12(E59A/F60A)/ | 130 | nM | 10 | Fur texture, reduced activity | None |
| anti-PD-1(D100G) | | | 50 | Fur texture, reduced activity | None |
| IL-12(E59A/F60A)/ | 910 | nM | 10 | Fur texture (moderate) | None |
| anti-PD-1(D100R) | | | 50 | Fur texture, reduced activity | None |
| IL-12(E59A/F60A)/ | 2300 | nM | 10 | None | None |

| | | | | |
|---|---|---|---|---|
| anti-PD-1(N99G) | | 50 | Fur texture (moderate) | None |
| IL-12(E59A/F60A)/ | N/A | 10 | None | None |
| anti-PD-1(N99M) | | 50 | Fur texture (moderate) | None |

Due to wildtype nivolumab's high binding affinity to PD-1 (K_{d}˜10⁸-10⁻⁹ M), IL-12(E59/F60A)/anti-PD-1(WT) most likely binds and stimulates (via the cytokine activity) any PD-1 positive cell. This would include activated T-cells and NK cells, which would result in cytokine release syndrome. In contrast, IL-12/anti-PD-1 based immunocytokines with reduced binding affinity to hPD-1 can only bind a smaller population of PD-1 positive cells, particulary cells with very high PD-I expression levels, such as exhausted T-cells. The data shown here is consistent with the data from the in vitro PBMC IFN-γ release assay in Example 27. These findings indicate that reducing the PD-1 binding affinity of anti-PD-1 antigen-binding domain to a Kₐ of between about 10⁻⁶-10⁻⁷ M (see, e.g., D100G, D100R, N99G in anti-PD-1 heavy chain) can greatly improve the safety of IL-12/anti-PD-1 immunocytokines, while retaining therapeutic efficacy.

### Example 29: Increasing the Binding Affinity of PD-L1 and PD-L2 does not Increase Toxicity of IL-12/PD-L1-Fc and IL-12/PD-L2-Fc Immunocytokines

As shown in Examples 14-17, replacing anti-PD-1 antigen-binding fragment with PD-L2 extracellular domain in IL-12 immunocytokines can significantly reduce toxicity, likely due to stimulated PD-1 inhibitory immune checkpoint signaling upon PD-L2-PD-1 binding, which created an immunosuppression signal that “balances” against the immunostimulating/pro-inflammatory activity of IL-12. To investigate if safety profiles of these “balancing” constructs can be further improved, IL-12 immunocytokines comprising PD-L1 or PD-L2 extracellular domain with increased PD-1 binding affinity were constructed, in order to enhance PD-1 immunosuppression signal.

Generation of PD-L1 Variants with Increased PD-1 Binding Affinity

Wildtype PD-L1 has a binding affinity for PD-1 of about 10⁵-10⁻⁶ M, which is lower than that of nivolumab(Kd≈10⁻⁸-10⁻⁹ M). To increase the affinity for PD-1, PD-L1 mutants were generated. Mutations were introduced into the extracellular domain of wildtype PD-L1 with amino acid positions relative to SEQ ID NO: 249. These mutant PD-L1 extracellular domains were then fused to an Fc fragment via a hinge region to construct parental PD-L1-Fc constructs. To construct PD-L1-Fc heterodimer, one polypeptide chain comprises a hinge region comprising SEQ ID NO: 52, and an Fc domain subunit comprising SEQ ID NO: 61; the other polypeptide chain comprises a hinge region comprising SEQ ID NO: 51, and an Fc domain subunit comprising SEQ ID NO: 62. Mutation constructs were named in the format of PD-L1(mut)-Fc.

A description of the mutations made and PD-1 binding affinities (measured in PD-L1-Fc format) are shown in Table 20. Binding affinity for each PD-L1(mut)-Fc was calibrated based on PD-L1(wt)-Fc binding affinity. N/A indicates non-detectable PD-1 binding. These results indicate that all PD-L1(mut) achieved about 4-60 fold increase in PD-1 binding affinity compared to wildtype PD-L1. Among these, PD-L1(I54Q/E58M/R113T/M115L/S117A/G119K) (PD-L1(mut2)), PD-L1(I54Q/E58M/R113T/M115L/G119K) (PD-L1(mut6)), and PD-L1(I54Q/E58M/R113T/M115L/S117A) (PD-L1(mut7)) showed the highest fold increase in affinity for PD-1 as compared to wildtype PD-Li.

TABLE 20
PD-1 binding affinities of various PD-L1 mutants

| | Affinity (Kd) human | | Affinity (Kd) mouse | |
|---|---|---|---|---|
| PD-L1 mutations | PD-1 | | PD-1 | |
| None | 7500 | nM | 5100 | nM |
| (PD-L1(WT); SEQ ID NO: 250) | | | | |
| E58M/R113T/M115L/S117A/G119K | N/A | | N/A | |
| (PD-L1(mut1); SEQ ID NO: 255) | | | | |
| I54Q/E58M/R113T/M115L/S117A/G119K | 150 | nM | 120 | nM |
| (PD-L1(mut2); SEQ ID NO: 256) | | | | |
| I54Q/R113T/M115L/S117A/G119K | 910 | nM | 820 | nM |
| (PD-L1(mut3); SEQ ID NO: 257) | | | | |
| I54Q/E58M/M115L/S117A/G119K | 1090 | nM | 980 | nM |
| (PD-L1(mut4); SEQ ID NO: 258) | | | | |
| I54Q/E58M/R113T/S117A/G119K | 1203 | nM | 1100 | nM |
| (PD-L1(mut5); SEQ ID NO: 259) | | | | |
| I54Q/E58M/R113T/M115L/G119K | 555 | nM | 420 | nM |
| (PD-L1(mut6); SEQ ID NO: 260) | | | | |
| I54Q/E58M/R113T/M115L/S117A | 98 | nM | 110 | nM |
| (PD-L1(mut7); SEQ ID NO: 261) | | | | |

Generation of PD-L2 Variants with Increased PD-1 Binding Affinity

PD-L2 has a binding affinity for PD-1 of about 10⁻⁶-10⁻⁷ M, which is lower than that of nivolumab(Kd≈10⁻⁸-10⁻⁹ M). To increase the affinity for PD-1, PD-L2 mutants were generated. Mutations were introduced into the extracellular domain of wildtype PD-L2 with amino acid positions relative to SEQ ID NO: 175. These mutant PD-L2 extracellular domains were then fused to an Fc fragment via a hinge region to construct parental PD-L2-Fc constructs. To construct PD-L2-Fc heterodimer, one polypeptide chain comprises a hinge region comprising SEQ ID NO: 52, and an Fc domain subunit comprising SEQ ID NO: 61; the other polypeptide chain comprises a hinge region comprising SEQ ID NO: 51, and an Fc domain subunit comprising SEQ ID NO: 62. Mutation constructs were named in the format of PD-L2(mut)-Fc.

A description of the mutations made and PD-1 binding affinities (measured in PD-L2-Fc format) are shown in Table 21. Binding affinity for each PD-L2(mut)-Fc was calibrated based on PD-L2(wt)-Fc binding affinity. These results indicate that all PD-L2(mut) achieved about 2-5 fold increase in PD-1 binding affinity compared to wildtype PD-L2. Among these, PD-L2(S58V) (PD-L2(mut2)) and PD-L2(T56V/S58V/Q60L) (PD-L2(mut4)) showed the highest fold increase in affinity for PD-1 as compared to wildtype PD-L2.

TABLE 21
PD-1 binding affinities of various PD-L2 mutants

| | | |
|---|---|---|
| | Affinity (Kd) | Affinity (Kd) |
| PD-L2 Mutations | human PD-1 | mouse PD-1 |

| | | | |
|---|---|---|---|
| None | 1200 | nM | 980 nM |
| (PD-L2(WT); SEQ ID NO: 176) | | | |
| T56V | 520 | nM | 430 nM |
| (PD-L2(mut1); SEQ ID NO: 262) | | | |
| S58V | 350 | nM | 230 nM |
| (PD-L2(mut2); SEQ ID NO: 263) | | | |
| Q60L | 490 | nM | 320 nM |
| (PD-L2(mut3); SEQ ID NO: 264) | | | |
| T56V/S58V/Q60L | 255 | nM | 220 nM |
| (PD-L2(mut4); SEQ ID NO: 265) | | | |

Construction of IL-12/PD-L1-Fc and IL-12/PD-L2-Fc Immunocytokines with Increased Affinity for PD-1

Similarly as described in Example 15, heterodimeric PD-L1(mut)-Fc or PD-L2(mut)-Fc generated herein were used as parental antigen-binding proteins to construct IL-12 immunocytokines that bind PD-1. Single chain IL-12(E59A/F60A) variant was placed at the N′ of the hinge of one polypeptide chain within the parental PD-L1(mut)-Fc or PD-L2(mut)-Fc heterodimers.

Briefly, IL-12(E59A/F60A)/PD-L2(wt)-Fc immunocytokine (“construct #29”) comprises one IL-12 fusion polypeptide comprising SEQ ID NO: 186 (from N′ to C′: PD-L2 extracellular domain (SEQ ID NO: 176)—GSG linker (SEQ ID NO: 203)—single chain IL-12(E59A/F60A) variant (SEQ ID NO: 36)—hinge (SEQ ID NO: 52)—Fc domain subunit (SEQ ID NO: 61)); and one pairing polypeptide comprising SEQ ID NO: 185 (from N′ to C′: PD-L2 extracellular domain (SEQ ID NO: 176)—GSG linker—hinge (SEQ ID NO: 51)—Fc domain subunit (SEQ ID NO: 62)). IL-12(E59A/F60A)/PD-L2(mut)-Fc immunocytokine comprises one IL-12 fusion polypeptide (from N′ to C′: PD-L2(mut) extracellular domain—GGGGSGGG linker (SEQ ID NO: 334)—single chain IL-12(E59A/F60A) variant—GGGGSGGG linker (SEQ ID NO: 334)—hinge (SEQ ID NO: 52)—Fc domain subunit (SEQ ID NO: 61)); and one pairing polypeptide (from N′ to C′: PD-L2(mut) extracellular domain—GGGGSGGG linker (SEQ ID NO: 334)—hinge (SEQ ID NO: 51)—Fc domain subunit (SEQ ID NO: 62)).

IL-12(E59A/F60A)/PD-L1(wt)-Fc immunocytokine comprises one IL-12 fusion polypeptide (from N′ to C′: PD-L1(wt) extracellular domain (SEQ ID NO: 250)—GGGGSGGG linker (SEQ ID NO: 334)—single chain IL-12(E59A/F60A) variant—GGGGSGGG linker (SEQ ID NO: 334)—hinge (SEQ ID NO: 52)—Fc domain subunit (SEQ ID NO: 61)); and one pairing polypeptide (from N′ to C′: PD-L1(wt) extracellular domain (SEQ ID NO: 250)—GGGGSGGG linker (SEQ ID NO: 334)—hinge (SEQ ID NO: 51)—Fc domain subunit (SEQ ID NO: 62)). IL-12(E59A/F60A)/PD-L1(mut)-Fc immunocytokine comprises one IL-12 fusion polypeptide (from N′ to C′: PD-L1(mut) extracellular domain—GGGGSGGG linker (SEQ ID NO: 334)—single chain IL-12(E59A/F60A) variant—GGGGSGGG linker (SEQ ID NO: 334)—hinge (SEQ ID NO: 52)—Fc domain subunit (SEQ ID NO: 61)); and one pairing polypeptide (from N′ to C′: PD-L1(mut) extracellular domain—GGGGSGGG linker (SEQ ID NO: 334)—hinge (SEQ ID NO: 51)—Fc domain subunit (SEQ ID NO: 62)).

To test the safety profiles of the IL-12 immunocytokines constructed with increased affinity to PD-1, wildtype C57 mice (5-6 weeks age, 20 g females) were injected with 10 mg/kg or 50 mg/kg (per injection) of IL-12(E59A/F60A)/PD-L1(mut2)-Fc imunocytokine or IL-12(E59A/F60A)/PD-L2(mut2)-Fc immunocytokine, as these two constructs showed similar PD-1 binding affinity to both human and mouse PD-1 (˜10⁻⁷ M). IL-12(E59A/F60A)/PD-L1(wt)-Fc imunocytokine or IL-12(E59A/F60A)/PD-L2(wt)-Fc immunocytokine served as control. A total of four injections were given on Days 0, 4, 8, and 12. Mice were monitored daily for mortality and four toxicity symptoms: i) fur texture, ii) reduced activity, iii) morbidity, and iv) weight loss.

As can be seen from Table 22, increasing binding affinity to PD-1 does not significantly affect the safety profiles of IL-12(E59A/F60A)/PD-L1-Fc immunocytokine or IL-12(E59A/F60A)/PD-L2-Fc immunocytokine. These results show that IL-12 immunocytokines comprising mutant versions of PD-L1 and PD-L2 with increased binding affinities to PD-1 retain the safety profile of wildtype IL-12/PD-L1-Fc and IL-12/PD-L2-Fc immunocytokines. This may be applied to other PD-L1-Fc or PD-L2-Fc based immunocytokines as well, to construct other immunocytokines (e.g., IL-2 immunocytokines).

TABLE 22
In vivo toxicity of IL-12/PD-L2 immunocytokines

| | Affinity | | | |
|---|---|---|---|---|
| Construct | (Kd) | Dose | Toxicity | Deaths |
| (cytokine fusion chain sequence) | mPD-1 | (mg/kg) | Symptoms | in group |
| IL-12(E59A/F60A)/PD-L1(WT)-Fc | 5100 nM | 10 | None | None |
| | | 50 | None | None |
| IL-12(E59A/F60A)/PD-L1 | 120 nM | 10 | None | None |
| (I54Q/E58M/R113T/M115L/S117A/G119K)-Fc | | 50 | Fur texture | None |
| (“IL-12(E59A/F60A)/PD-L1 | | | (moderate) | |
| (mut2)-Fc”; SEQ ID NO: 277) | | | | |
| IL-12(E59A/F60A)/PD-L2(WT)-Fc | 980 nM | 10 | None | None |
| (IL-12(E59A/F60A)/PD-L2(mut2)-Fc; | | 50 | None | None |
| (construct #29; SEQ ID NO: 186) | | 50 | None | None |
| IL-12(E59A/F60A)/PD-L2(S58V)-Fc | 230 nM | 10 | None | None |
| SEQ ID NO: 293 | | | | |

### Example 30: Generation of IL-2/PD-L1 Immunocytokines with IL-2 Biological Activity Directed to PD-1-Positive Cells

Certain cytokines have synergistic action, such as IL-12 and IL-2, IL-12 and IFN-γ. To reduce toxicity of IL-2 and immunocytokines thereof, two sets of IL-2 mutations were generated: mutations within IL-2 domain that interacts with IL2Ru (CD25) (R38D/K43E/E61R), and mutations within IL-2 domain that interacts with IL2Ry (CD132) (L18R, Q22E, Q126T, S130R, or any combinations thereof). See Table 23.

Heterodimeric PD-L1(mut2)-Fc was used as the parental PD-1 binding protein. First polypeptide chain comprises SEQ ID NO: 281 (N′ to C′: PD-L1(mut2) extracellular domain (SEQ ID NO: 256)—GGGGSGGG linker (SEQ ID NO: 334)—hinge (SEQ ID NO: 52)—Fc domain subunit (SEQ ID NO: 61)), second polypeptide chain comprises SEQ ID NO: 283 (N′ to C′: PD-L1(mut2) extracellular domain (SEQ ID NO: 256)—GGGGSGGG linker (SEQ ID NO: 334) —hinge (SEQ ID NO: 51)—Fc domain subunit (SEQ ID NO: 62)). To construct IL-2 immunocytokines, IL-2 variant was placed between the PD-L1(mut2) extracellular domain and the hinge. Briefly, IL-2(mut)/PD-L1(mut2)-Fc immunocytokine comprises one IL-2 fusion polypeptide (from N′ to C′: PD-L1(mut2) extracellular domain (SEQ ID NO: 256)—GGGSG linker (SEQ ID NO: 209) —IL-2(mut) variant—GGGGSGGG linker (SEQ ID NO: 334)—hinge (SEQ ID NO: 51)—Fc domain subunit (SEQ ID NO: 62)); and one pairing polypeptide SEQ ID NO: 281.

HEK-Blue™ IL-2 Cells and HEK-PD-1-IL-2 cells were used to assess IL-2 signal activation activity of the constructs, as described in Examples 1-3. As can be seen from Table 23, IL-2(R38D/K43E/E61R)/PD-L1(mut2)-Fc with IL-2 mutations only in CD25 binding domain (R38D/K43E/E61R) still retained about 16% IL-2 activity based on HEK-IL-2 assay (no PD-1 binding), while IL-2 immunocytokines further carrying IL-2 mutations in the CD132 binding domain significantly decreased IL-2 activity based on HEK-IL-2 assay, in the absence of PD-1 binding. Notably, IL-2 activity of some of the IL-2 immunocytokines further carrying CD132 binding domain mutations (see constructs comprising SEQ ID NO: 285, SEQ ID NO: 286, or SEQ ID NO: 287 chain) can be partialy rescued when binding to PD-1 (based on HEK-IL-2-PD-1 assay). S130 may be crutial for IL-2 activity, as IL-2 immunocytokine further carrying S130R mutation in CD132 binding domain (see construct comprising SEQ ID NO: 288 chain), in combination with other IL-2 mutations, fail to exhibit any IL-2 activity even in the presence of PD-1 binding.

TABLE 23
IL-2 biological activity of IL-2/anti-PD-1 immunocytokines

| | Non-fusion | IL-2 fusion or | | | | | |
|---|---|---|---|---|---|---|---|
| | polypeptide | non-fusion | IL-2 | CD25 | CD132 | | |
| | PD- | polypeptide PD- | SEQ | binding | binding | | HEK- |
| | L1(mut2)-Fc | L1(mut2)-Fc | ID | site | site | HEK- | IL-2- |
| Construct | SEQ ID NO: | SEQ ID NO: | NO: | mutations | mutations | IL-2 | PD-1 |
| rIL2 | / | / | | / | / | 100% | 100% |
| PD-L1(mut2)-Fc | 281 | 283 | / | / | / | <0.1% | <0.1% |
| IL- | 281 | 327 | 2 | R38D/ | / | 16% | 76% |
| 2(R38D/K43E/E61R)/ | | | | K43E/E61R | | | |
| PD-L1(mut2)-Fc | | | | | | | |
| IL- | 281 | 285 | 251 | R38D/ | L18R/Q22E | 4% | 35% |
| 2(L18R/Q22E/R38D/ | | | | K43E/E61R | | | |
| K43E/E61R)/PD-L1(mut2)- | | | | | | | |
| Fc | | | | | | | |
| IL- | 281 | 286 | 252 | R38D/ | Q126T | 2% | 20% |
| 2(R38D/K43E/E61R/ | | | | K43E/E61R | | | |
| Q126T)/PD-L1(mut2)-Fc | | | | | | | |
| IL- | 281 | 287 | 253 | R38D/ | L18R/Q22E/ | <0.1% | 5% |
| 2(L18R/Q22E/R38D/ | | | | K43E/E61R | Q126T | | |
| K43E/E61R/Q126T)/PD- | | | | | | | |
| L1(mut2)-Fc | | | | | | | |
| IL- | 281 | 288 | 254 | R38D/ | L18R/Q22E/ | <0.1% | <0.1% |
| 2(L18R/Q22E/R38D/ | | | | K43E/E61R | Q126T/ | | |
| K43E/E61R/Q126T/S130R)/ | | | | | S130R | | |
| PD-L1(mut2)-Fc | | | | | | | |

### Example 31: Generation of IL-2/IL-12/PD-L1 Immunocytokines with IL-2 and IL-12 Biological Activity Directed to PD-1-Positive Cells

Certain cytokines have synergistic action, such as IL-12 and IL-2. As shown in Examples above, IL-12(E59A/F60A)/PD-L1-Fc immunocytokine (hinge region) showed PD-1 binding dependent IL-12 activity. To investigate whether immunocytokines can be constructed with synergistic IL-12 and IL-2 activity, while retaining PD-1 binding dependent cytokine activity, different configurations of immunocytokines were constructed. Heterodimeric PD-L1(mut2)-Fc was used as parental PD-1 binding fusion protein (constructed in Example 29). Set I: one polypeptide chain comprises single chain IL-12(E59A/F60A) polypeptide positioned at the hinge region of PD-L1(mut2)-Fc (see SEQ ID NO: 277 constructed in Example 29); the pairing polypeptide chain does not comprise IL-2 moiety (control; SEQ ID NO: 283 constructed in Example 29), or comprises IL-2 variant (either with L18R/Q22E/R38D/K43E/E61R mutation (SEQ ID NO: 251), or with R38D/K43E/E61R/Q126T mutation (SEQ ID NO: 252)) positioned at the hinge region of PD-L1(mut2)-Fc. These immunocytokines are named in the format of IL-2/IL-12(E59A/F60A)/PD-L1(mut2)-Fc. Set II: one polypeptide chain comprises IL-12(E59A/F60A) fused to the C′ of PD-L1(mut2)-Fc via GGGGSGGG linker (see SEQ ID NO: 279); the pairing polypeptide chain does not comprise IL-2 moiety (control; SEQ ID NO: 283 constructed in Example 29), or comprises IL-2 variant (either with L18R/Q22E/R38D/K43E/E61R mutation (SEQ ID NO: 251), or with R38D/K43E/E61R/Q126T mutation (SEQ ID NO: 252)) positioned at the hinge region of PD-L1(mut2)-Fc. These immunocytokines are named in the format of IL-2/PD-L1(mut2)-Fc/IL-12(E59A/F60A), indicating that IL-12 moiety is at the C′ of Fc. See Table 24 for construct sequences.

HEK-Blue™ IL-2 Cells and HEK-PD-1-IL-2 cells were used to assess IL-2 signal activation activity of the constructs, as described in Examples 1-3. HEK-Blue™ IL-12 Cells and HEK-PD-1-IL-12 cells were used to assess IL-12 signal activation activity of the constructs, as described in Example 4.

As can be seen from Table 24, IL-12(E59A/F60A)/PD-L1(mut2)-Fc (hinge fusion) did not have detectable IL-12 activity in the absence of PD-1 binding, while PD-1 binding rescued the IL-12 activity to 24%. When IL-12(E59A/F60A) was placed at C′ of Fc as PD-L1(mut2)-Fc/IL-12(E59A/F60A), IL-12 activity was about 10%-2% in the absence of PD-1 binding, and IL-12 activity was further rescued by PD-1 binding (˜25%), to similar extent as the IL-12 hinge fusion.

By adding on IL-2 in the pairing chain at the hinge region, for both IL-12 hinge fusion and C′ fusion formats, IL-2 activity was about 20-40 in the absence of PD-1 binding, but was rescued to about 200DN35% by PD-1 binding.

These data indicate that IL-i2 and IL-2 can both retain PD-1-binding dependent activity when constructed in trispecific immunocytokine format. Further, IL-12 and IL-2 moeities did not have significant negative impact on each other's activity.

TABLE 24
IL-2 and IL-12 biological activity of IL-2/IL-12/PD-L1-Fc
immunocytokines and IL-2/PD-L1-Fc/IL-12 immunocytokines

| | | IL-2 fusion or | | | | |
|---|---|---|---|---|---|---|
| | IL-12 fusion | non-fusion | | HEK- | | HEK- |
| | polypeptide | polypeptide | | IL-2- | HEK- | IL-12- |
| Construct | SEQ ID NO: | SEQ ID NO: | HEK-IL-2 | PD-1 | IL-12 | PD-1 |
| rIL-2 | / | / | 100% | 100% | / | / |
| rIL-12 | / | / | / | / | 100% | 100% |
| IL-12(E59A/F60A)/PD- | 277 | 283 (no IL-2) | <0.1% | <0.1% | <0.2% | 24% |
| L1(mut2)-Fc | (IL-12 hinge) | | | | | |
| IL- | 277 | 285 | 4% | 35% | <0.2% | 19% |
| 2(L18R/Q22E/R38D/K43E/ | (IL-12 hinge) | | | | | |
| E61R)/IL-12(E59A/F60A)/PD- | | | | | | |
| L1(mut2)-Fc | | | | | | |
| IL- | 277 | 286 | 2% | 20% | <0.2% | 25% |
| 2(R38D/K43E/E61R/Q126T)/ | (IL-12 hinge) | | | | | |
| IL-12(E59A/F60A)/PD- | | | | | | |
| L1(mut2)-Fc | | | | | | |
| PD-L1(mut2)-Fc/IL- | 279 | 283 (no IL-2) | <0.1% | <0.1% | 1% | 25% |
| 12(E59A/F60A) | (IL-12 at C′) | | | | | |
| IL- | 279 | 285 | 3% | 32% | 2% | 27% |
| 2(L18R/Q22E/R38D/K43E/ | (IL-12 at C′) | | | | | |
| E61R)/PD-L1(mut2)-Fc/IL- | | | | | | |
| 12(E59A/F60A) | | | | | | |
| IL- | 279 | 286 | 3% | 31% | 1% | 30% |
| 2(R38D/K43E/E61R/Q126T)/ | (IL-12 at C′) | | | | | |
| PD-L1(mut2)-Fc/IL- | | | | | | |
| 12(E59A/F60A) | | | | | | |

### Example 32: Placing IL-12 Moiety at the Hinge Region can Greatly Improve Safety Profiles of IL-12/PD-L1-Fc Immunocytokines and IL-2/IL-12/PD-L1-Fc Immunocytokines

PD-L1(mut2)-Fc constructed in Example 29 was used as parental PD-1 binding fusion protein, as it showed similar PD-1 binding affinity in both human and mice.

To test safety profiles in vivo, a mouse single-chain IL-12 variant (SEQ ID NO: 330) with E59A/F60A mutations in the p40 subunit and a p35 wildtype subunit was similarly constructed as described herein: from N′ to C′ p40(E59A/F60A)-GGPGGGGSGGGSGGGG linker (SEQ ID NO: 335)-p35(wt) (human IL-12 does not function in mouse). Two sets of IL-12 fusion polypeptides were constructed, similar to Example 31. Set I: one polypeptide chain comprises single chain mIL-12(E59A/F60A) polypeptide positioned at the hinge region of PD-L1(mut2)-Fc (see SEQ ID NO: 328); the pairing polypeptide chain does not comprise IL-2 moiety (control; SEQ ID NO: 283 constructed in Example 29), or comprises IL-2 variant (with R38D/K43E/E61R mutation (SEQ ID NO: 2), L18R/Q22E/R38D/K43E/E61R mutation (SEQ ID NO: 251), or with R38D/K43E/E61R/Q126T mutation (SEQ ID NO: 252)) positioned at the hinge region of PD-L1(mut2)-Fc. These immunocytokines are named in the format of IL-2/IL-12(E59A/F60A)/PD-L1(mut2)-Fc. Set II: one polypeptide chain comprises single-chain mIL-12(E59A/F60A) fused to the C′ of PD-L1(mut2)-Fc via GGGGSGGG linker (see SEQ ID NO: 279); the pairing polypeptide chain does not comprise IL-2 moiety (control; SEQ ID NO: 283 constructed in Example 29), or comprises IL-2 variant (with R38D/K43E/E61R mutation (SEQ ID NO: 2), L18R/Q22E/R38D/K43E/E61R mutation (SEQ ID NO: 251) positioned at the hinge region of PD-L1(mut2)-Fc. These immunocytokines are named in the format of IL-2/PD-L1(mut2)-Fc/IL-12(E59A/F60A), indicating that IL-12 moiety is at the C′ of Fc. See Table 24 for construct sequences. A Control Set did not have any IL-12 moiety fusion to PD-L1(mut2)-Fc (SEQ ID NO: 281).

To test safety profiles of these constructs, wild-type C57 mice (5-6 weeks age, weight 20 g, female) were injected with PBS (control), or the immunocytokines (10 mg/kg per injection) described herein. A total of 4 injections were given every 4 days. Mice were monitored for death and toxicity symptoms, such as fur texture, reduced activity, and weight loss. 48 hours after the 2^{nd} injection, blood was collected and serum concentrations of IFN-γ was measured.

As shown in Table 25, immunocytokines comprising IL-2 additional mutations in CD132 binding domain (L18R/Q22E, or Q126T) in addition to R38D/K43E/E61R in CD25 binding domain, showed much greater safety profiles compared to those without CD132 binding domain mutations (see constructs comprising SEQ ID NO: 327 chain), irrespective ofifthe L-12 moiety is at C′ or at hinge.

Immunocytokines with IL-12 at the C′ of Fc, IL-2(mut)/PD-L1(mut2)-Fc/mTL-12(E59A/F6 pA) showed higher toxicity compared to DL-12 positioned at hinge region (IL-2(mut)/mIL-12(E59A/F6A)/PD-L1(mut2)-Fc). IL-2(mut)/PD-L1(mut2)-Fc/mL-12(E59A/F60A) also induced much higher (20-30 folds) cytokine release (see IFN-γ level) compared to IL-12 hinge fusion design.

Taken together, our in vivo and in vitro data presented herein suggested that immunocytokines with cytokine (e.g., IL-12 or variant thereof) positioned at the hinge region can significantly improve the safety profile, even when more cytokines with synergistic acttions are present in the same construct (e.g., IL-2/IL-12/PD-L-Fc). Mutations in cytokines to reduce their activities, and/or mutations in antigen-binding domain (e.g., anti-PD-1 or PD-Li, or PD-L2), can further improve safety and/or therapeutic efficacy of the constructs.

TABLE 25
IL-2 and IL-12 biological activity of IL-2/IL-12/PD-L1-Fc
immunocytokines and IL-2/PD-L1-Fc/IL-12 immunocytokines

| | IL-12 fusion or | IL-2 fusion or | | | | | |
|---|---|---|---|---|---|---|---|
| | non-fusion | non-fusion | | | | | |
| | polypeptide SEQ | polypeptide SEQ | Dose | Toxicity | Deaths | Blood | |
| Construct | ID NO: | ID NO: | (mg/kg) | Symptoms | in group | IFN-γ | |
| PBS | / | / | / | None | None | 5 | pg/ml |
| PD-L1(mut2)-Fc | 281 | 283 | 10 mg/kg | None | None | 6 | pg/ml |
| | (no IL-12) | (no IL-2) | | | | | |
| IL- | 281 | 327 | 10 mg/kg | Fur texture, | None | 45 | pg/ml |
| 2(R38D/K43E/E61R)/ | (no IL-12) | | | reduced | | | |
| PD-L1(mut2)-Fc | | | | activity | | | |
| IL- | 281 | 285 | 10 mg/kg | None | None | 32 | pg/ml |
| 2(L18R/Q22E/R38D/ | (no IL-12) | | | | | | |
| K43E/E61R)/PD- | | | | | | | |
| L1(mut2)-Fc | | | | | | | |
| IL- | 281 | 286 | 10 mg/kg | None | None | 24 | pg/ml |
| 2(R38D/K43E/E61R/ | (no IL-12) | | | | | | |
| Q126T)/PD-L1(mut2)-Fc | | | | | | | |
| mIL- | 328 | 283 | 10 mg/kg | None | None | 80 | pg/ml |
| 12(E59A/F60A)/PD- | (mIL-12 at hinge) | (no IL-2) | | | | | |
| L1(mut2)-Fc | | | | | | | |
| IL- | 328 | 327 | 10 mg/kg | Fur texture, | None | 90 | pg/ml |
| 2(R38D/K43E/E61R)/ | (mIL-12 at hinge) | | | reduced | | | |
| mIL- | | | | activity | | | |
| 12(E59A/F60A)/PD- | | | | | | | |
| L1(mut2)-Fc | | | | | | | |
| IL- | 328 | 285 | 10 mg/kg | None | None | 64 | pg/ml |
| 2(L18R/Q22E/R38D/ | (mIL-12 at hinge) | | | | | | |
| K43E/E61R)/mIL- | | | | | | | |
| 12(E59A/F60A)/PD- | | | | | | | |
| L1(mut2)-Fc | | | | | | | |
| IL- | 328 | 286 | 10 mg/kg | None | None | 45 | pg/ml |
| 2(R38D/K43E/E61R/ | (mIL-12 at hinge) | | | | | | |
| Q126T)/mIL- | | | | | | | |
| 12(E59A/F60A)/PD- | | | | | | | |
| L1(mut2)-Fc | | | | | | | |
| PD-L1(mut2)-Fc/mIL- | 329 | 283 | 10 mg/kg | Fur texture | None | 1400 | pg/ml |
| 12(E59A/F60A) | (mIL-12 at C′) | (no IL-2) | | (moderate) | | | |
| IL- | 329 | 327 | 10 mg/kg | Fur texture, | 1/5 | 5600 | pg/ml |
| 2(R38D/K43E/E61R)/ | (mIL-12 at C′) | | | reduced | | | |
| PD-L1(mut2)-Fc/mIL- | | | | activity | | | |
| 12(E59A/F60A) | | | | | | | |
| IL- | 329 | 285 | 10 mg/kg | Fur texture, | None | 1800 | pg/ml |
| 2(L18R/Q22E/R38D/ | (mIL-12 at C′) | | | reduced | | | |
| K43E/E61R)/PD- | | | | activity | | | |
| L1(mut2)-Fc/mIL- | | | | | | | |
| 12(E59A/F60A) | | | | | | | |
| IL- | 329 | 286 | 10 mg/kg | Fur texture | None | 2100 | pg/ml |
| 2(R38D/K43E/E61R/ | (mIL-12 at C′) | | | (moderate) | | | |
| Q126T)/PD-L1(mut2)- | | | | | | | |
| Fc/mIL- | | | | | | | |
| 12(E59A/F60A) | | | | | | | |

SEQUENCE LISTING
(wildtype mature human IL-2)
SEQ ID NO: 1
APTSSSTKKTQLQLEHLLLDLQMILNGINNYKNPKLTRMLTFKFYMPKKATELKHLQCLEEELKPLEEVLN
LAQSKNFHLRPRDLISNINVIVLELKGSETTFMCEYADETATIVEFLNRWITFCQSIISTLT
(IL-2 mutant R38D/K43E/E61R)
SEQ ID NO: 2

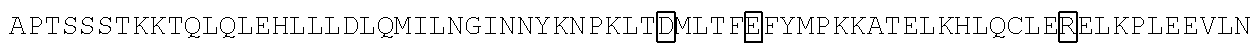

LAQSKNFHLRPRDLISNINVIVLELKGSETTFMCEYADETATIVEFLNRWITFCQSIISTLT
(wildtype mature human IFN-α2b)
SEQ ID NO: 3
CDLPQTHSLGSRRTLMLLAQMRKISLFSCLKDRHDFGFPQEEFGNQFQKAETIPVLHEMIQQIFNLFSTKDSS
AAWDETLLDKFYTELYQQLNDLEACVIQGVGVTETPLMKEDSILAVRKYFQRITLYLKEKKYSPCAWEVV
RAEIMRSFSLSTNLQESLRSKE
(IFN-α2b mutant L30A)
SEQ ID NO: 4

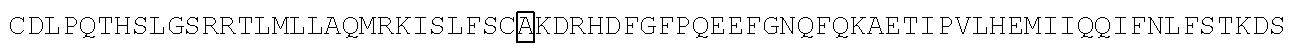

SAAWDETLLDKFYTELYQQLNDLEACVIQGVGVTETPLMKEDSILA VRKYFQRITLYLKEKKYSPCAWEV
VRAEIMRSFSLSTNLQESLRSKE
(IFN-α2b mutant K31A)
SEQ ID NO: 5

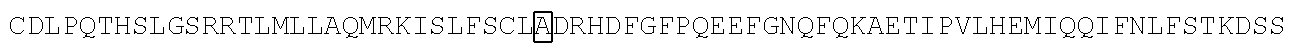

AAWDETLLDKFYTELYQQLNDLEACVIQGVGVTETPLMKEDSILAVRKYFQRITLYLKEKKYSPCAWEVV
RAEIMRSFSLSTNLQESLRSKE
(IFN-α2b mutant D32A)
SEQ ID NO: 6

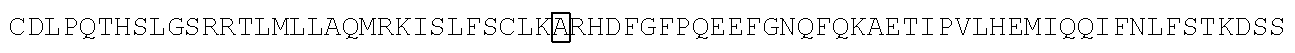

AAWDETLLDKFYTELYQQLNDLEACVIQGVGVTETPLMKEDSILAVRKYFQRITLYLKEKKYSPCAWEVV
RAEIMRSFSLSTNLQESLRSKE
(IFN-α2b mutant R33A)
SEQ ID NO: 7

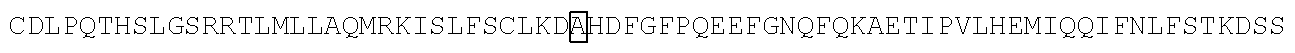

AAWDETLLDKFYTELYQQLNDLEACVIQGVGVTETPLMKEDSILAVRKYFQRITLYLKEKKYSPCAWEVV
RAEIMRSFSLSTNLQESLRSKE
(IFN-α2b mutant H34A)
SEQ ID NO: 8

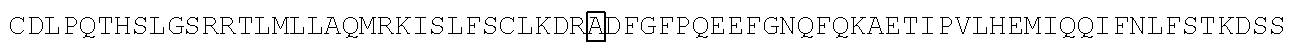

AAWDETLLDKFYTELYQQLNDLEACVIQGVGVTETPLMKEDSILAVRKYFQRITLYLKEKKYSPCAWEVV
RAEIMRSFSLSTNLQESLRSKE
(IFN-α2b mutant D35A)
SEQ ID NO: 9

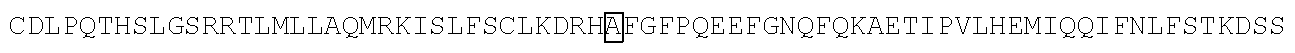

AAWDETLLDKFYTELYQQLNDLEACVIQGVGVTETPLMKEDSILAVRKYFQRITLYLKEKKYSPCAWEVV
RAEIMRSFSLSTNLQESLRSKE
(wildtype mature human IFN-γ monomer)
SEQ ID NO: 10
QDPYVKEAENLKKYFNAGHSDVADNGTLFLGILKNWKEESDRKIMQSQIVSFYFKLFKNFKDDQSIQKSVE
TIKEDMNVKFFNSNKKKRDDFEKLTNYSVTDLNVQRKAIHELIQVMAELSPAAKTGKRKRSQMLFRG
(IFN-γ mutant S20A/D21A monomer)
SEQ ID NO: 11

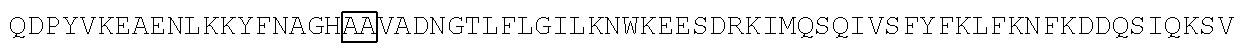

ETIKEDMNVKFFNSNKKKRDDFEKLTNYSVTDLNVQRKAIHELIQVMAELSPAAKTGKRKRSQMLFRG
(IFN-γ mutant V22A/A23S monomer)
SEQ ID NO: 12

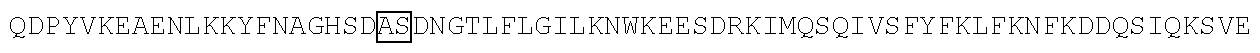

TIKEDMNVKFFNSNKKKRDDFEKLTNYSVTDLNVQRKAIHELIQVMAELSPAAKTGKRKRSQMLFRG
(IFN-γ mutant A23V monomer)
SEQ ID NO: 13

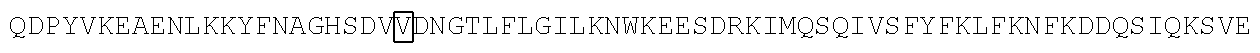

TIKEDMNVKFFNSNKKKRDDFEKLTNYSVTDLNVQRKAIHELIQVMAELSPAAKTGKRKRSQMLFRG
(IFN-γ mutant D24A/N25A monomer)
SEQ ID NO: 14

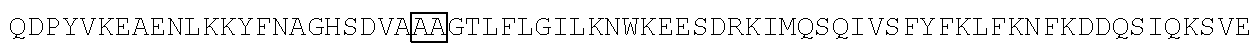

TIKEDMNVKFFNSNKKKRDDFEKLTNYSVTDLNVQRKAIHELIQVMAELSPAAKTGKRKRSQMLFRG
IFN-γ mutant A23E/D24E/N25K monomer)
SEQ ID NO: 15

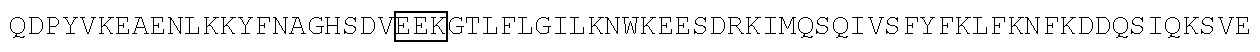

TIKEDMNVKFFNSNKKKRDDFEKLTNYSVTDLNVQRKAIHELIQVMAELSPAAKTGKRKRSQMLFRG
IFN-γ mutant A23Q monomer)
SEQ ID NO: 16

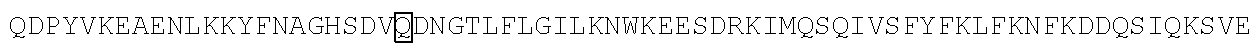

TIKEDMNVKFFNSNKKKRDDFEKLTNYSVTDLNVQRKAIHELIQVMAELSPAAKTGKRKRSQMLFRG
(IFN-γ mutant D21K monomer)
SEQ ID NO: 17

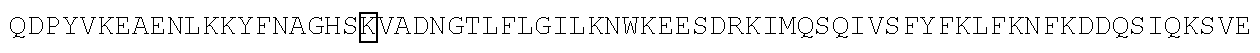

TIKEDMNVKFFNSNKKKRDDFEKLTNYSVTDLNVQRKAIHELIQVMAELSPAAKTGKRKRSQMLFRG
(single-chain “wildtype” IFN-γ homodimer; linker is bolded; wildtype IFN-γ monomer is
italicized)
SEQ ID NO: 18
QDPYVKEAENLKKYFNAGHSDVADNGTLFLGILKNWKEESDRKIMQSQIVSFYFKLFKNFKDDQSIQKSVETIKE
DMNVKFFNSNKKKRDDFEKLTNYSVTDLNVORKAIHELIQVMAELSPAAKTGKRKRSOMLFRGFEGGGSGGGG
SGGGGSGGGGSQDPYVKEAENLKKYFNAGHSDVADNGTLFLGILKNWKEESDRKIMQSQIVSFYFKLFKNFKD
DQSIQKSVETIKEDMNVKFFNSNKKKRDDFEKLTNYSVTDLNVQRKAIHELIQVMAELSPAAKTGKRKRSQMLFR
G
(single-chain IFN-γ mutant A23V homodimer; linker is bolded; IFN-γ mutant monomer is
italicized)
SEQ ID NO: 19

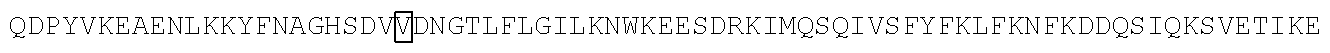

DMNVKFFNSNKKKRDDFEKLTNYSVTDLNVORKAIHELIQVMAELSPAAKTGKRKRSQMLFRGFEGGGSGGGG

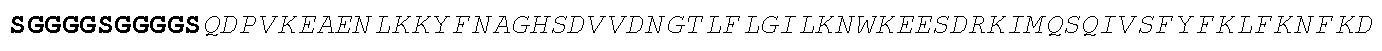

DQSIQKSVETIKEDMNVKFFNSNKKKRDDFEKLTNYSVTDLNVORKAIHELIQVMAELSPAAKTGKRKRSQMLFR
G
(wildtype mature human IL-10 monomer)
SEQ ID NO: 20
SPGQGTQSENSCTHFPGNLPNMLRDLRDAFSRVKTFFQMKDQLDNLLLKESLLEDFKGYLGCQALSEMIQF
YLEEVMPQAENQDPDIKAHVNSLGENLKTLRLRLRRCHRFLPCENKSKAVEQVKNAFNKLQEKGIYKAMS
EFDIFINYIEAYMTMKIRN
(IL-10 mutant R24A monomer)
SEQ ID NO: 21

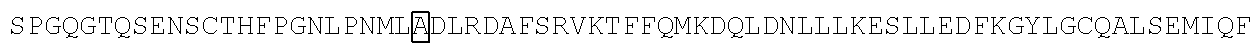

YLEEVMPQAENQDPDIKAHVNSLGENLKTLRLRLRRCHRFLPCENKSKAVEQVKNAFNKLQEKGIYKAMS
EFDIFINYIEAYMTMKIRN
(IL-10 mutant D25A/L26A monomer)
SEQ ID NO: 22

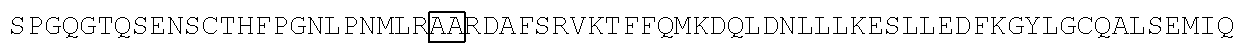

FYLEEVMPQAENQDPDIKAHVNSLGENLKTLRLRLRRCHRFLPCENKSKAVEQVKNAFNKLQEKGIYKAM
SEFDIFINYIEAYMTMKIRN
(IL-10 mutant R27A monomer)
SEQ ID NO: 23

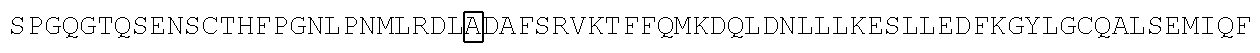

YLEEVMPQAENQDPDIKAHVNSLGENLKTLRLRLRRCHRFLPCENKSKAVEQVKNAFNKLQEKGIYKAMS
EFDIFINYIEAYMTMKIRN
(IL-10 mutant D28A/A29S monomer)
SEQ ID NO: 24

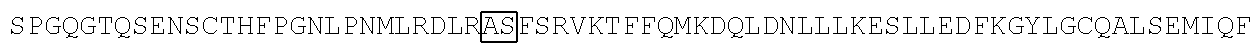

YLEEVMPQAENQDPDIKAHVNSLGENLKTLRLRLRRCHRFLPCENKSKAVEQVKNAFNKLQEKGIYKAMS
EFDIFINYIEAYMTMKIRN
(IL-10 mutant F30A/S31A monomer)
SEQ ID NO: 25

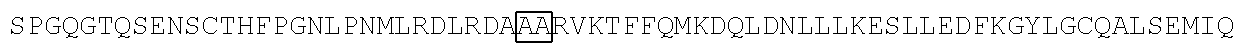

FYLEEVMPQAENQDPDIKAHVNSLGENLKTLRLRLRRCHRFLPCENKSKAVEQVKNAFNKLQEKGIYKAM
SEFDIFINYIEAYMTMKIRN
(IL-10 mutant R32A monomer)
SEQ ID NO: 26

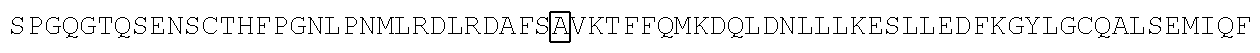

YLEEVMPQAENQDPDIKAHVNSLGENLKTLRLRLRRCHRFLPCENKSKAVEQVKNAFNKLQEKGIYKAMS
EFDIFINYIEAYMTMKIRN
(single-chain “wildtype” IL-10 homodimer; linker is bolded; wildtype IL-10 monomer is
italicized)
SEQ ID NO: 27
SPGQGTQSENSCTHFPGNLPNMLRDLRDAFSRVKTFFQMKDQLDNLLLKESLLEDFKGYLGCQALSEMIQFYLE
EVMPQAENQDPDIKAHVNSLGENLKTLRLRLRRCHRFLPCENKSKAVEQVKNAFNKLOEKGIYKAMSEFDIFINYI
EAYMTMKIRNFEGGGSGGGGSGGGGSGGGGSSPGQGTQSENSCTHFPGNLPNMLRDLRDAFSRVKTFFQMK
DQLDNLLLKESLLEDFKGYLGCQALSEMIQFYLEEVMPQAENQDPDIKAHVNSLGENLKTLRLRLRRCHRFLPCE
NKSKAVEQVKNAFNKLQEKGIYKAMSEFDIFINYIEAYMTMKIRN
(single-chain IL-10 mutant R27A homodimer; linker is bolded; IL-10 mutant monomer is
italicized)
SEQ ID NO: 28

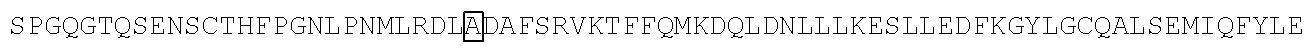

EVMPQAENQDPDIKAHVNSLGENLKTLRLRLRRCHRFLPCENKSKAVEQVKNAFNKLQEKGIYKAMSEFDIFINYI

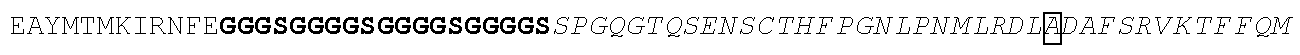

KDQLDNLLLKESLLEDFKGYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN
(wildtype mature human IL-12A (p35) subunit)
SEQ ID NO: 29
RNLPVATPDPGMFPCLHHSQNLLRAVSNMLQKARQTLEFYPCTSEEIDHEDITKDKTSTVEACLPLELTKNE
SCLNSRETSFITNGSCLASRKTSFMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
(wildtype mature human IL-12B (p40) subunit)
SEQ ID NO: 30
IWELKKDVYVVELDWYPDAPGEMVVLTCDTPEEDGITWTLDQSSEVLGSGKTLTIQVKEFGDAGQYTCHK
GGEVLSHSLLLLHKKEDGIWSTDILKDQKEPKNKTFLRCEAKNYSGRFTCWWLTTISTDLTFSVKSSRGSSD
PQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASIS
VRAQDRYYSSSWSEWASVPCS
(IL-12B (p40) mutant E59A/F60A subunit)
SEQ ID NO: 31

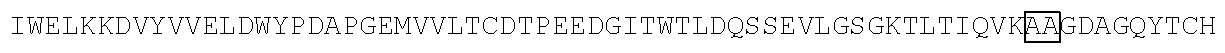

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
(IL-12B (p40) mutant E59A subunit)
SEQ ID NO: 32

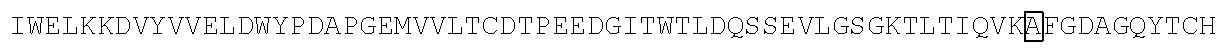

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
(IL-12B (p40) mutant F60A subunit)
SEQ ID NO: 33

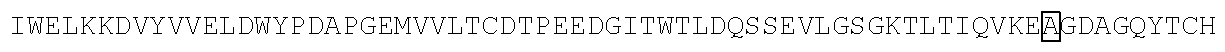

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
(IL-12B (p40) mutant G64A subunit)
SEQ ID NO: 34

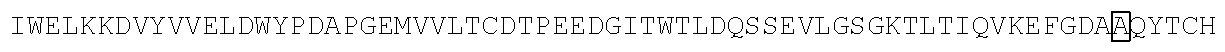

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
(single-chain “wildtype” IL-12 heterodimer IL-12B (wt p40)-linker-IL-12A (wt p35); linker is
bolded)
SEQ ID NO: 35
IWELKKDVYVVELDWYPDAPGEMVVLTCDTPEEDGITWTLDQSSEVLGSGKTLTIQVKEFGDAGQYTCHKGGEV
LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECOEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKTSTVEACLPLELTKNESCLNSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKLCILLHAFRIRAVTIDRVMSYLNAS
(single-chain IL-12 mutant heterodimer IL-12B (p40 E59A/F60A)-linker-IL-12A (wt p35);
linker is bolded; IL-12 subunits are italicized)
SEQ ID NO: 36

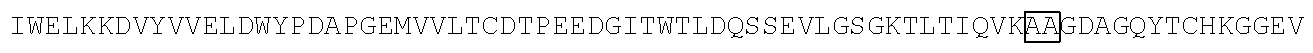

LSHSLLLLHKKEDGIWSTDILKDQKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSONLLRAVSNMLQKARQTLEFYPCTSEEI
DHEDITKDKTSTVEACLPLELTKNESCLNSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKLCILLHAFRIRAVTIDRVMSYLNAS
(wildtype mature human IL-23A (p19) subunit)
SEQ ID NO: 37
RAVPGGSSPAWTQCQQLSQKLCTLAWSAHPLVGHMDLREEGDEETTNDVPHIQCGDGCDPQGLRDNSQF
CLORIHQGLIFYEKLLGSDIFTGEPSLLPDSPVGQLHASLLGLSQLLQPEGHHWETQQIPSLSPSQPWQRLLL
RFKILRSLQAFVAVAARVFAHGAATLSP
(single-chain “wildtype” IL-23 heterodimer IL-12B (wt p40)-linker-IL-23A (wt p19); linker is
bolded; IL-23 subunits are italicized)
SEQ ID NO: 38
IWELKKDVYVVELDWYPDAPGEMVVLTCDTPEEDGITWTLDQSSEVLGSGKTLTIQVKEFGDAGQYTCHKGGEV
LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAODRYYSSSWSEWASV
PCSGGGGSGGGGSGGGGSGGGGSGRAVPGGSSPAWTQCQQLSQKLCTLAWSAHPLVGHMDLREEGDEETT
NDVPHIQCGDGCDPQGLRDNSQFCLQRIHQGLIFYEKLLGSDIFTGEPSLLPDSPVGQLHASLLGLSQLLQPEGH
HWETQQIPSLSPSQPWQRLLLRFKILRSLQAFVAVAARVFAHGAATLSP
(single-chain IL-23 mutant heterodimer IL-12B (p40 E59A/F60A)-linker-IL-23A (wt p19);
linker is bolded; IL-23 subunits are italicized)
SEQ ID NO: 39

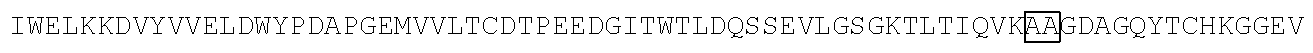

LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRAVPGGSSPAWTQCQQLSQKLCTLAWSAHPLVGHMDLREEGDEETT
NDVPHIQCGDGCDPQGLRDNSQFCLQRIHQGLIFYEKLLGSDIFTGEPSLLPDSPVGQLHASLLGLSQLLOPEGH
HWETQQIPSLSPSQPWQRLLLRFKILRSLQAFVAVAARVFAHGAATLSP
(hinge)
SEQ ID NO: 40
EPKSCDKTHTCPPCPAPELLGGP
(hinge)
SEQ ID NO: 41

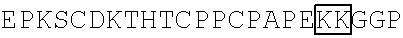

(hinge)
SEQ ID NO: 42

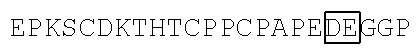

(hinge)
SEQ ID NO: 43

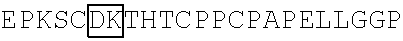

(hinge)
SEQ ID NO: 44

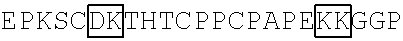

(hinge)
SEQ ID NO: 45

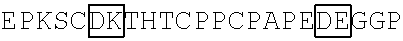

(hinge)
SEQ ID NO: 46
ERKCCVECPPCPAPPVAGP
(hinge)
SEQ ID NO: 47
ESKYGPPCPSCPAPEFLGGP
(hinge, e.g., hinge N′ portion)
SEQ ID NO: 48
EPKSCDK
(hinge, e.g., hinge N' portion)
SEQ ID NO: 49
EPKSC
(hinge, e.g., hinge C' portion)
SEQ ID NO: 50
DKTHTCPPCPAPELLGGP
(hinge, e.g., hinge C' portion)
SEQ ID NO: 51

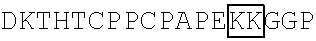

(hinge, e.g., hinge C' portion)
SEQ ID NO: 52

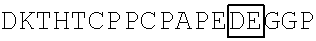

(hinge)
SEQ ID NO: 53
DKTHT
(hinge, e.g., hinge N' portion)
SEQ ID NO: 54

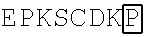

(hinge)
SEQ ID NO: 55

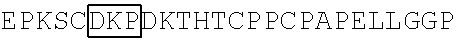

(hinge)
SEQ ID NO: 56

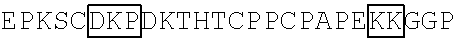

(hinge)
SEQ ID NO: 57

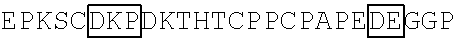

(hinge)
SEQ ID NO: 58
ESKYGPPCPPCPAPEFLGGP
(hinge)
SEQ ID NO: 59

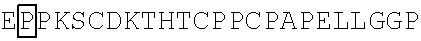

(wildtype human IgG1 Fc)
SEQ ID NO: 60
SVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCL VKGFYPSDIAV
EWESNGQPENNYKTTPPVLDSDGSFFLYSKLTVDKSRWQQGNVFSCSVMHEALHNHYTQKSLSLSPGK
(IgG1 Fc mutant1 T350V/L351Y/S400E/F405A/Y407V)
SEQ ID NO: 61
SVFLFPPKPKDTLMISRTPEVTCVVVDVSHEDPEVKFNWYVDGVEVHNAKTKPREEQYNSTYRVVSVLTV

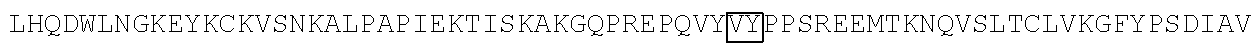

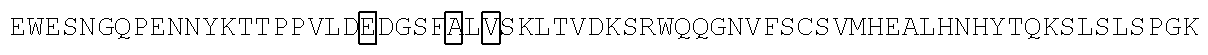

(IgG1 Fc mutant2 T350V/T366L/N390R/K392M/T394W)
SEQ ID NO: 62
SVFLFPPKPKDTLMISRTPEVTCVVVDVSHEDPEVKFNWYVDGVEVHNAKTKPREEQYNSTYRVVSVLTV

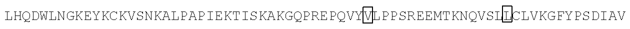

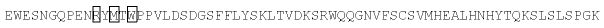

(wildtype human IgG4 Fc)
SEQ ID NO: 63
SVFLFPPKPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
(IgG1 Fc mutant)
SEQ ID NO: 64

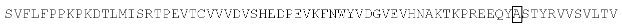

LHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
(IgG1 Fc mutant)
SEQ ID NO: 65

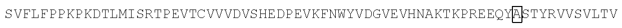

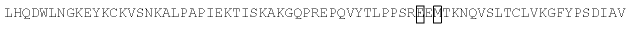

EWESNGQPENNYKTTPPVLDSDGSFFLYSKLTVDKSRWQQGNVFSCSVMHEALHNHYTQKSLSLSPGK
(IgG1 Fc mutant)
SEQ ID NO: 66
SVFLFPPKPKDTLMISRTPEVTCVVVDVSHEDPEVKFNWYVDGVEVHNAKTKPREEQYNSTYRVVSVLTV

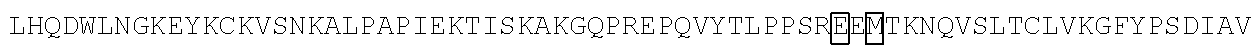

EWESNGQPENNYKTTPPVLDSDGSFFLYSKLTVDKSRWQQGNVFSCSVMHEALHNHYTQKSLSLSPGK
(ibalizumab/Trogarzo anti-CD4 Ab HC-CDR1)
SEQ ID NO: 67
GYTFTSYVIH
(ibalizumab/Trogarzo anti-CD4 Ab HC-CDR2)
SEQ ID NO: 68
YNDGTDYDEKFKG
(ibalizumab/Trogarzo anti-CD4 Ab HC-CDR3)
SEQ ID NO: 69
EKDNYATGAWFAY
(ibalizumab/Trogarzo anti-CD4 Ab LC-CDR1)
SEQ ID NO: 70
KSSQSLLYSTNQKNYLA
(ibalizumab/Trogarzo anti-CD4 Ab LC-CDR2)
SEQ ID NO: 71
WASTRES
(ibalizumab/Trogarzo anti-CD4 Ab LC-CDR3)
SEQ ID NO: 72
QQYYSYRT
(ibalizumab/Trogarzo anti-CD4 Ab VH; CDRs are underlined)
SEQ ID NO: 73
QVQLQQSGPEVVKPGASVKMSCKASGYTFTSYVIHWVRQKPGQGLDWIGYINPYNDGTDYDEKFKGKAT
LTSDTSTSTAYMELSSLRSEDTAVYYCAREKDNYATGAWFAYWGQGTLVTVSSAS
(ibalizumab/Trogarzo anti-CD4 Ab VL; CDRs are underlined)
SEQ ID NO: 74
DIVMTQSPDSLAVSLGERVTMNCKSSQSLLYSTNQKNYLAWYQQKPGQSPKLLIYWASTRESGVPDRFSGS
GSGTDFTLTISSVQAEDVAVYYCQQYYSYRTFGGGTKLEIK
(anti-CD4 Ab HC (IgG1 Fc mutant); VH is underlined; hinge is bolded)
SEQ ID NO: 75
QVQLQQSGPEVVKPGASVKMSCKASGYTFTSYVIHWVROKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNW
YVDGVEVHNAKTKPREEQYASTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
(ibalizumab/Trogarzo anti-CD4 Ab HC (IgG4 Fc); VH is underlined; hinge is bolded)
SEQ ID NO: 76
QVQLQQSGPEVVKPGASVKMSCKASGYTFTSYVIHWVROKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPCSRST
SESTAALGCLVKDYFPEPVTVSWNSGALTSGVHTFPAVLQSSGLYSLSSVVTVPSSSLGTKTYTCNVDHKPS
NTKVDKRVESKYGPPCPSCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQPREPQVYT
LPPSQEEMTKNQVSLTCLVKGFYPSDIA VEWESNGQPENNYKTTPPVLDSDGSFFLYSRLTVDKSRWQEGN
VFSCSVMHEALHNHYTQKSLSLSLGK
(ibalizumab/Trogarzo anti-CD4 Ab LC; VL is underlined)
SEQ ID NO: 77
DIVMTQSPDSLAVSLGERVTMNCKSSQSLLYSTNQKNYLAWYQQKPGQSPKLLIYWASTRESGVPDRFSGS
GSGTDFTLTISSVQAEDVAVYYCQQYYSYRTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTKS
FNRGEC
(anti-CD4 Ab HC (IgG1 Fc mutant2); VH is underlined; hinge is bolded)
SEQ ID NO: 78
QVQLQQSGPEVVKPGASVKMSCKASGYTFTSYVIHWVROKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS

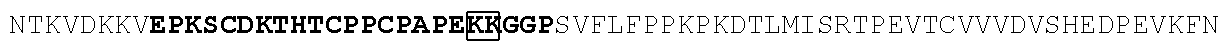

WYVDGVEVHNAKTKPREEQYNSTYRVVSVLTVLHQDWLNGKEYKCKVSNKALPAPIEKTISKAKGQPRE

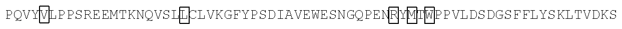

RWQQGNVFSCSVMHEALHNHYTQKSLSLSPGK
(anti-CD4 Ab VH-CH1-N' hinge portion-IL-2 mutant R38D/K43E/E61R-C' hinge portion-
SEQ ID NO: 79
IgG1 Fc mutant1; VH is underlined; hinge is bolded; IL-2 mutant is italicized)
QVQLQQSGPEVVKPGASVKMSCKASGYTFTSYVIHWVROKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS

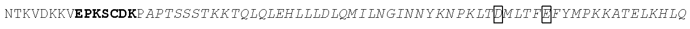

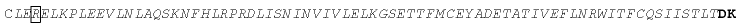

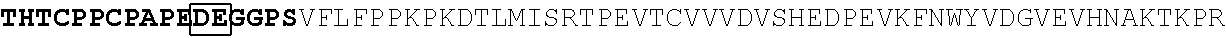

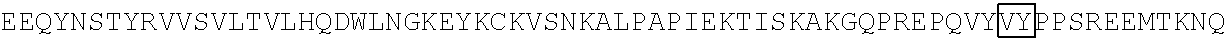

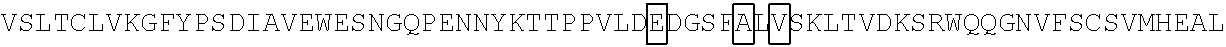

HNHYTQKSLSLSPGK
(anti-CD4 Ab VH-CH1-N' hinge portion-linker-IFN-a2b mutant L30A-C' hinge portion-
IgG1 Fc mutant1; VH is underlined; hinge is bolded; linker is bolded and underlined; IFN-a2b mutant is
italicized)
SEQ ID NO: 80
QVQLQQSGPEVVKPGASVKMSCKASGYTFTSYVIHWVRQKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS

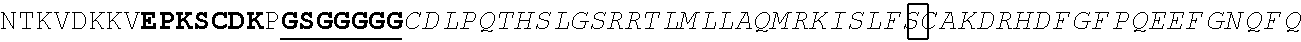

KAETIPVLHEMIQQIFNLFSTKDSSAAWDETLLDKFYTELYQQLNDLEACVIQGVGVTETPLMKEDSILAVRKYFQ

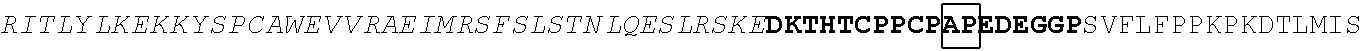

RTPEVTCVVVDVSHEDPEVKFNWYVDGVEVHNAKTKPREEQYNSTYRVVSVLTVLHQDWLNGKEYKCK

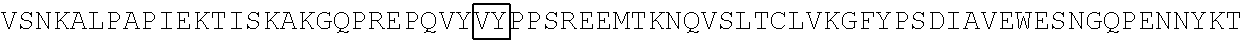

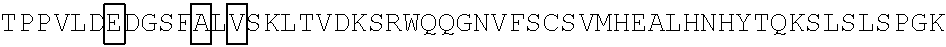

(anti-CD4 Ab VH-CH1-N' hinge portion-linker-single-chain IFN-γ mutant A23V
homodimer-C' hinge portion-IgG1 Fc mutant1; VH is underlined; hinge is bolded; linker is bolded and
underlined; IFN-γ mutant monomer is italicized)
SEQ ID NO: 81
QVQLQQSGPEVVKPGASVKMSCKASGYTFTSYVIHWVRQKPGQGLDWIGYINPYNDGTDYDEKFKGKAT
LTSDTSTSTAYMELSSLRSEDTAVYYCAREKDNYATGAWFAYWGOGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS

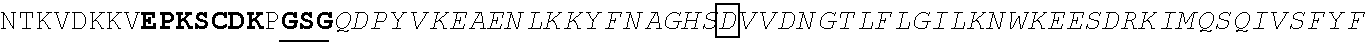

KLFKNFKDDQSIQKSVETIKEDMNVKFFNSNKKKRDDFEKLTNYSVTDLNVORKAIHELIQVMAELSPAAKTGKR

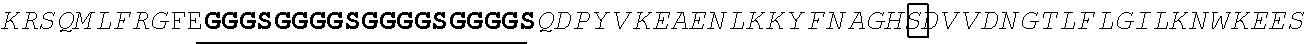

DRKIMQSQIVSFYFKLFKNFKDDQSIOKSVETIKEDMNVKFFNSNKKKRDDFEKLTNYSVTDLNVORKAIHELIQV

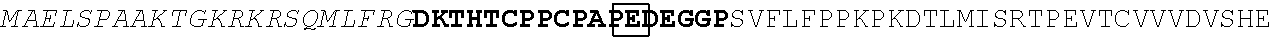

DPEVKFNWYVDGVEVHNAKTKPREEQYNSTYRVVSVLTVLHQDWLNGKEYKCKVSNKALPAPIEKTISK

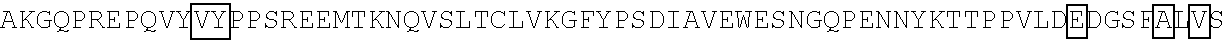

KLTVDKSRWQQGNVFSCSVMHEALHNHYTQKSLSLSPGK
(anti-CD4 Ab VH-CH1-N' hinge portion-linker-single-chain IL-10 mutant R27A homodimer-
C' hinge portion-IgG1 Fc mutant1; VH is underlined; hinge is bolded; linker is bolded and underlined;
IL-10 mutant monomer is italicized)
SEQ ID NO: 82
QVQLQQSGPEVVKPGASVKMSCKASGYTFTSYVIHWVRQKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS

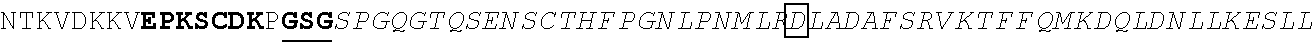

EDFKGYLGCQALSEMIQFYLEEVMPQAENQDPDIKAHVNSLGENLKTLRLRLRRCHRFLPCENKSKAVEQVKNA
FNKLOEKGIYKAMSEFDIFINYIEAYMTMKIRNFEGGGSGGGGSGGGGSGGGGSSPGQGTQSENSCTHFPGNL

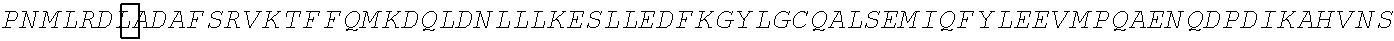

LGENLKTLRLRLRRCHRFLPCENKSKAVEQVKNAFNKLQEKGIYKAMSEFDIFINYIEAYMTMKIRNDKTHTCPP

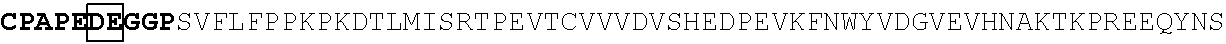

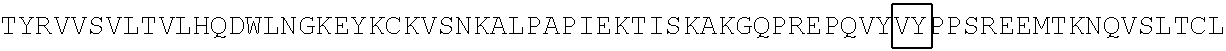

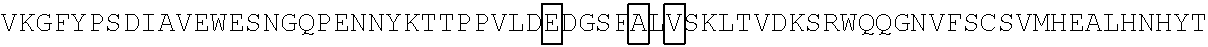

QKSLSLSPGK
(anti-CD4 Ab VH-CH1-N' hinge portion-linker-single-chain IL-12 mutant heterodimer IL-
12B (p40 E59A/F60A)-linker-IL-12A (wt p35)-C' hinge portion-IgG1 Fc mutant1; VH is underlined; hinge
is bolded; linker is bolded and underlined; IL-12 subunits are italicized)
SEQ ID NO: 83
QVQLQQSGPEVVKPGASVKMSCKASGYTFTSYVIHWVRQKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS
NTKVDKKVEPKSCDKPGSGIWELKKDVYVVELDWYPDAPGEMVVLTCDTPEEDGITWTLDQSSEVLGSGKTL

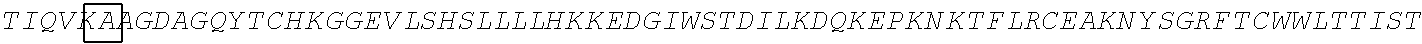

DLTFSVKSSRGSSDPOGVTCGAATLSAERVRGDNKEYEYSVECQEDSACPAAEESLPIEVMVDAVHKLKYENYTSS
FFIRDIIKPDPPKNLOLKPLKNSRQVEVSWEYPDTWSTPHSYFSLTFCVQVQGKSKREKKDRVFTDKTSATVICRK
NASISVRAQDRYYSSSWSEWASVPCSGGGGSGGGGSGGGGSGGGGSGRNLPVATPDPGMFPCLHHSQNLLRA
VSNMLQKARQTLEFYPCTSEEIDHEDITKDKTSTVEACLPLELTKNESCLNSRETSFITNGSCLASRKTSFMMALCL
SSIYEDLKMYQVEFKTMNAKLLMDPKRQIFLDQNMLAVIDELMQALNFNSETVPQKSSLEEPDFYKTKIKLCILLH

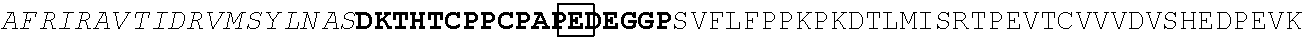

FNWYVDGVEVHNAKTKPREEQYNSTYRVVSVLTVLHQDWLNGKEYKCKVSNKALPAPIEKTISKAKGQP

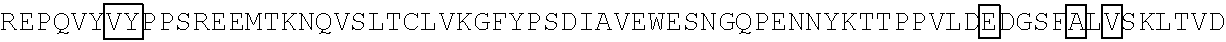

KSRWQQGNVFSCSVMHEALHNHYTQKSLSLSPGK
(anti-CD4 Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined; hinge
is bolded; linker is bolded and underlined; IL-23 subunits are italicized)
SEQ ID NO: 84
QVQLQQSGPEVVKPGASVKMSCKASGYTFTSYVIHWVROKPGQGLDWIGYINPYNDGTDYDEKFKGKAT
LTSDTSTSTAYMELSSLRSEDTAVYYCAREKDNYATGAWFAYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVNHKPS
NTKVDKKVEPKSCDKPGSGIWELKKDVYVVELDWYPDAPGEMVVLTCDTPEEDGITWTLDQSSEVLGSGKTL

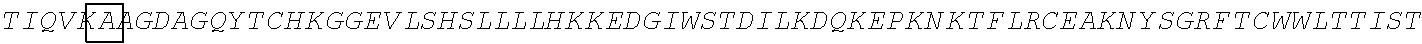

DLTFSVKSSRGSSDPQGVTCGAATLSAERVRGDNKEYEYSVECQEDSACPAAEESLPIEVMVDAVHKLKYENYTSS
FFIRDIIKPDPPKNLOLKPLKNSRQVEVSWEYPDTWSTPHSYFSLTFCVQVQGKSKREKKDRVFTDKTSATVICRK
NASISVRAQDRYYSSSWSEWASVPCSGGGGSGGGGSGGGGSGGGGSGRAVPGGSSPAWTQCQQLSQKLCTLA
WSAHPLVGHMDLREEGDEETTNDVPHIQCGDGCDPQGLRDNSQFCLQRIHQGLIFYEKLLGSDIFTGEPSLLPD
SPVGOLHASLLGLSQLLQPEGHHWETQQIPSLSPSQPWQRLLLRFKILRSLQAFVAVAARVFAHGAATLSPDKTH

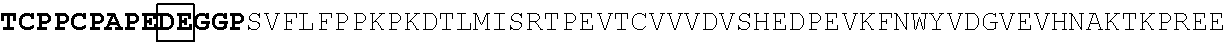

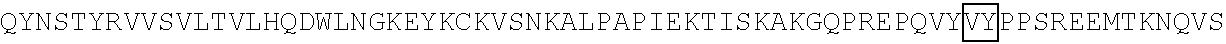

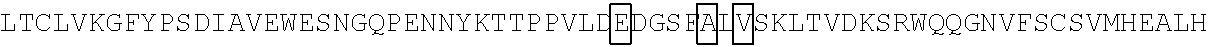

NHYTQKSLSLSPGK
anti-CD3ϵ Ab HC-CDR1)
SEQ ID NO: 85
GFTFSYFWMN
(anti-CD3ϵ Ab HC-CDR2)
SEQ ID NO: 86
EIRLRSDNYATHYAESVKG
(anti-CD3ϵ Ab HC-CDR3)
SEQ ID NO: 87
DWEFTY
(anti-CD3ϵ Ab LC-CDR1)
SEQ ID NO: 88
SASLSVSFMH
(anti-CD3ϵ Ab LC-CDR2)
SEQ ID NO: 89
RTSNLAS
(anti-CD3ϵ Ab LC-CDR3)
SEQ ID NO: 90
QQRSSDPP
(anti-CD3ϵ Ab VH; CDRs are underlined)
SEQ ID NO: 91
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSAS
(anti-CD3ϵ Ab VL; CDRs are underlined)
SEQ ID NO: 92
EIVMTQSPGTLSLSPGESATLSCSASLSVSFMHWFQQKPGHAPRLLIYRTSNLASGVPDRFSGSGSGTDFTLT
ISRLEPEDFAVYYCQQRSSDPPTFGQGTRLEI
(anti-CD3ϵ Ab HC (IgG1 Fc mutant); VH is underlined)
SEQ ID NO: 93
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHEDPEVKFNWYVDG
VEVHNAKTKPREEQYASTYRVVSVLTVLHQDWLNGKEYKCKVSNKALPAPIEKTISKAKGQPREPQVYTL
PPSRDELTKNQVSLTCL VKGFYPSDIAVEWESNGQPENNYKTTPPVLDSDGSFFLYSKLTVDKSRWQQGNV
FSCSVMHEALHNHYTQKSLSLSPGK
(anti-CD3ϵ Ab LC; VL is underlined)
SEQ ID NO: 94
EIVMTQSPGTLSLSPGESATLSCSASLSVSFMHWFQQKPGHAPRLLIYRTSNLASGVPDRFSGSGSGTDFTLT
ISRLEPEDFAVYYCQQRSSDPPTFGQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
(anti-CD3: Ab HC (IgG1 Fc mutant2); VH is underlined; hinge is bolded)
SEQ ID NO: 95
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

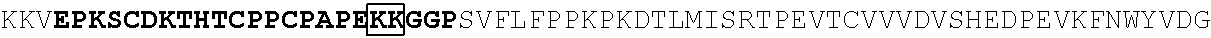

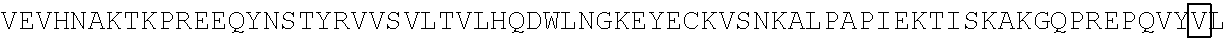

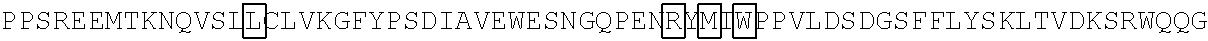

NVFSCSVMHEALHNHYTQKSLSLSPGK
(anti-CD3& Ab VH-CH1-N' hinge portion-IL-2 mutant R38D/K43E/E61R-C' hinge portion-
IgG1 Fc mutant1; VH is underlined; hinge is bolded; IL-2 mutant is italicized)
SEQ ID NO: 96
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

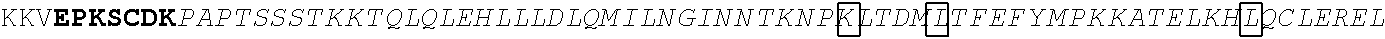

KPLEEVLNLAOSKNFHLRPRDLISNINVIVLELKGSETTFMCEYADETATIVEFLNRWITFCOSIISTLTDKTHTCPP

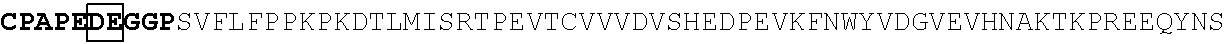

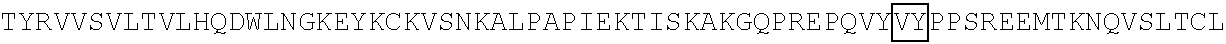

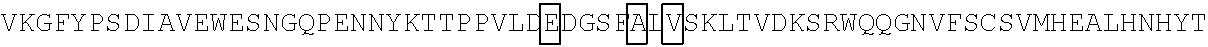

QKSLSLSPGK
(anti-CD3ϵ Ab VH-CH1-N' hinge portion-linker-IFN-a2b mutant L30A-C' hinge portion-
IgG1 Fc mutant1; VH is underlined; hinge is bolded; linker is bolded and underlined; IFN-a2b mutant
is italicized)
SEQ ID NO: 97
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

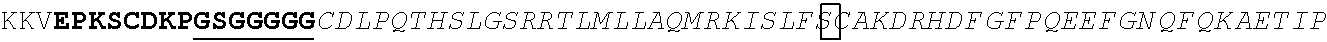

VLHEMIQQIFNLFSTKDSSAAWDETLLDKFYTELYQOLNDLEACVIQGVGVTETPLMKEDSILAVRKYFORITLYLK

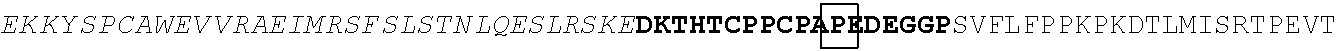

CVVVDVSHEDPEVKFNWYVDGVEVHNAKTKPREEQYNSTYRVVSVLTVLHQDWLNGKEYKCKVSNKAL

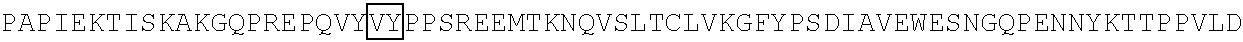

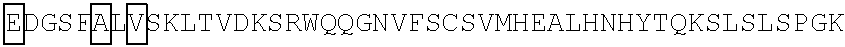

(anti-CD3& Ab VH-CH1-N' hinge portion-linker-single-chain IFN-γ mutant A23V
homodimer-C' hinge portion-IgG1 Fc mutant1; VH is underlined; hinge is bolded; linker is bolded
and underlined; IFN-γ mutant monomer is italicized)
SEQ ID NO: 98
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

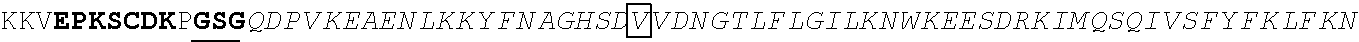

FKDDQSIQKSVETIKEDMNVKFFNSNKKKRDDFEKLTNYSVTDLNVORKAIHELIQVMAELSPAAKTGKRKRSQM

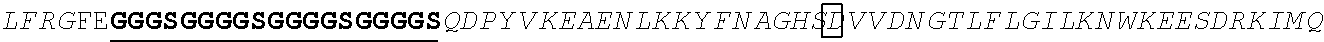

SQIVSFYFKLFKNFKDDQSIQKSVETIKEDMNVKFFNSNKKKRDDFEKLTNYSVTDLNVORKAIHELIQVMAELSP

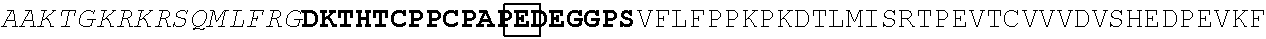

NWYVDGVEVHNAKTKPREEQYNSTYRVVSVLTVLHQDWLNGKEYKCKVSNKALPAPIEKTISKAKGQPR

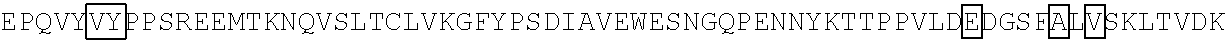

SRWQQGNVFSCSVMHEALHNHYTQKSLSLSPGK
(anti-CD3ϵ Ab VH-CH1-N' hinge portion-linker-single-chain IL-10 mutant R27A
homodimer-C' hinge portion-IgG1 Fc mutant1; VH is underlined; hinge is bolded; linker is bolded
and underlined; IL-10 mutant monomer is italicized)
SEQ ID NO: 99
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

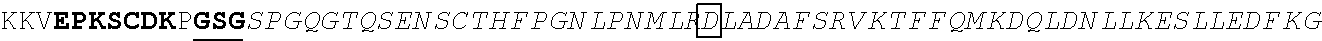

YLGCQALSEMIQFYLEEVMPQAENQDPDIKAHVNSLGENLKTLRLRLRRCHRFLPCENKSKAVEQVKNAFNKLQ
EKGIYKAMSEFDIFINYIEAYMTMKIRNFEGGGSGGGGSGGGGSGGGGSSPGQGTQSENSCTHFPGNLPNML

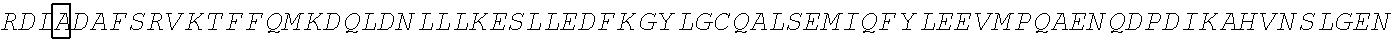

LKTLRLRLRRCHRFLPCENKSKAVEQVKNAFNKLOEKGIYKAMSEFDIFINYIEAYMTMKIRNDKTHTCPPCPAP

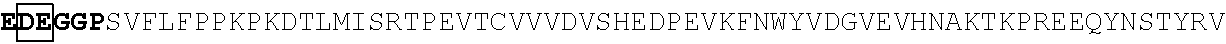

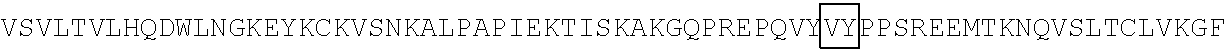

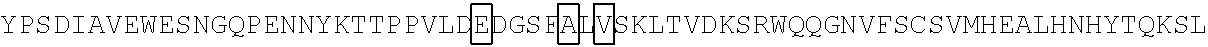

SLSPGK
(anti-CD3& Ab VH-CH1-N' hinge portion-linker-single-chain IL-12 mutant heterodimer IL-
12B (p40 E59A/F60A)-linker-IL-12A (wt p35)-C' hinge portion-IgG1 Fc mutant1; VH is underlined;
hinge is bolded; linker is bolded and underlined; IL-12 subunits are italicized)
SEQ ID NO: 100
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

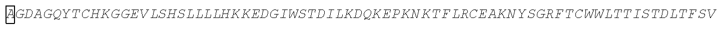

KSSRGSSDPQGVTCGAATLSAERVRGDNKEYEYSVECQEDSACPAAEESLPIEVMVDAVHKLKYENYTSSFFIRDII
KPDPPKNLOLKPLKNSROVEVSWEYPDTWSTPHSYFSLTFCVQVQGKSKREKKDRVFTDKTSATVICRKNASISVR
AQDRYYSSSWSEWASVPCSGGGGSGGGGSGGGGSGGGGSGRNLPVATPDPGMFPCLHHSONLLRAVSNMLQ
KARQTLEFYPCTSEEIDHEDITKDKTSTVEACLPLELTKNESCLNSRETSFITNGSCLASRKTSFMMALCLSSIYEDL
KMYQVEFKTMNAKLLMDPKRQIFLDQNMLAVIDELMQALNFNSETVPQKSSLEEPDFYKTKIKLCILLHAFRIRA

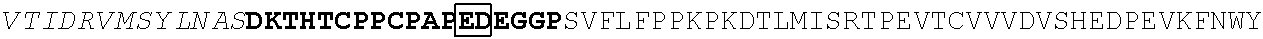

VDGVEVHNAKTKPREEQYNSTYRVVSVLTVLHQDWLNGKEYKCKVSNKALPAPIEKTISKAKGQPREPQV

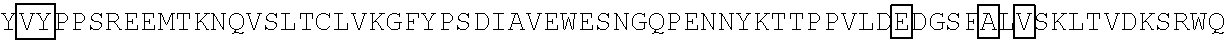

QGNVFSCSVMHEALHNHYTQKSLSLSPGK
(anti-CDϵ Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined;
hinge is bolded; linker is bolded and underlined; IL-23 subunits are italicized)
SEQ ID NO: 101
EVQLVESGGGLVQPGRSLRLSCTASGFTFSYFWMNWVRQAPGKGLEWVGEIRLRSDNYATHYAESVKGR
FTISRDDSKSIAYLQMNSLKTEDTALYYCTRDWEFTYWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVD

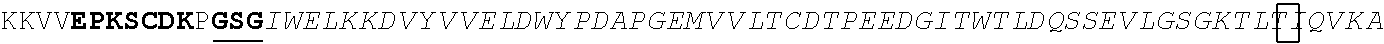

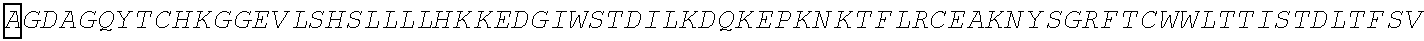

KSSRGSSDPQGVTCGAATLSAERVRGDNKEYEYSVECQEDSACPAAEESLPIEVMVDAVHKLKYENYTSSFFIRDII
KPDPPKNLQLKPLKNSRQVEVSWEYPDTWSTPHSYFSLTFCVQVQGKSKREKKDRVFTDKTSATVICRKNASISVR
AQDRYYSSSWSEWASVPCSGGGGSGGGGSGGGGSGGGGSGRAVPGGSSPAWTQCQQLSQKLCTLAWSAHPL
VGHMDLREEGDEETTNDVPHIQCGDGCDPQGLRDNSQFCLQRIHQGLIFYEKLLGSDIFTGEPSLLPDSPVGQL
HASLLGLSQLLQPEGHHWETQQIPSLSPSQPWQRLLLRFKILRSLQAFVAVAARVFAHGAATLSPDKTHTCPPCP

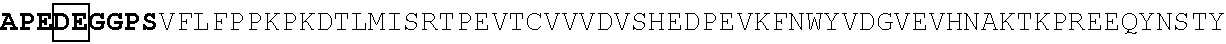

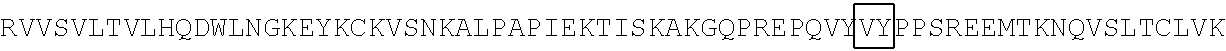

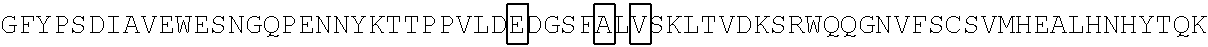

SLSLSPGK
(nivolumab/Opdivo anti-PD-1 Ab VH)
SEQ ID NO: 102
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSAS
(nivolumab/Opdivo anti-PD-1 Ab VL)
SEQ ID NO: 103
EIVLTQSPATLSLSPGERATLSCRASQSVSSYLAWYQQKPGQAPRLLIYDASNRATGIPARFSGSGSGTDFTL
TISSLEPEDFAVYYCQQSSNWPRTFGQGTKVEI
(anti-PD-1 Ab HC (IgG1 Fc mutant); VH is underlined)
SEQ ID NO: 104
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEV
HNAKTKPREEQYASTYRVVSVLTVLHQDWLNGKEYKCKVSNKALPAPIEKTISKAKGQPREPQVYTLPPSR
DELTKNQVSLTCLVKGFYPSDIA VEWESNGQPENNYKTTPPVLDSDGSFFLYSKLTVDKSRWQQGNVFSCS
VMHEALHNHYTQKSLSLSPGK
(nivolumab/Opdivo anti-PD-1 Ab HC; VH is underlined; hinge is bolded)
SEQ ID NO: 105
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPSQEEM
TKNQVSLTCLVKGFYPSDIA VEWESNGQPENNYKTTPPVLDSDGSFFLYSRLTVDKSRWQEGNVFSCSVM
HEALHNHYTQKSLSLSLGK
(nivolumab/Opdivo anti-PD-1 Ab LC; VL is underlined)
SEQ ID NO: 106
EIVLTQSPATLSLSPGERATLSCRASQSVSSYLAWYQQKPGQAPRLLIYDASNRATGIPARFSGSGSGTDFTL
TISSLEPEDFAVYYCQQSSNWPRTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
(anti-PD-1 Ab HC (IgG1 Fc mutant2); VH is underlined; hinge is bolded)
SEQ ID NO: 107
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

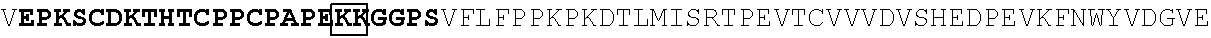

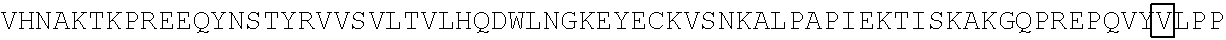

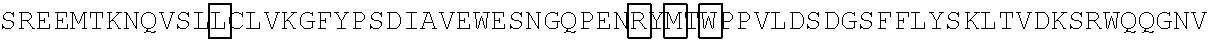

FSCSVMHEALHNHYTQKSLSLSPGK
(anti-PD-1 Ab VH-CH1-N' hinge portion-IL-2 mutant R38D/K43E/E61R-C' hinge portion-
IgG1 Fc mutant1; VH is underlined; hinge is bolded; IL-2 mutant is italicized)
SEQ ID NO: 108
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

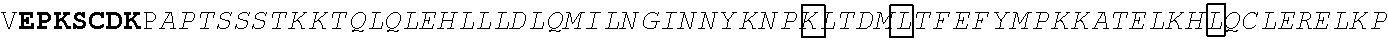

LEEVLNLAQSKNFHLRPRDLISNINVIVLELKGSETTFMCEYADETATIVEFLNRWITFCOSIISTLTDKTHTCPPCP

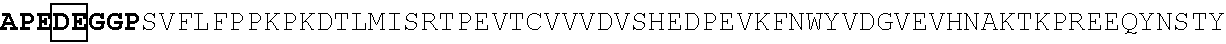

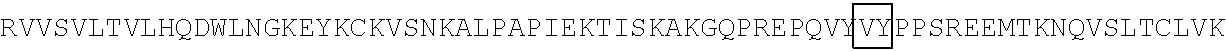

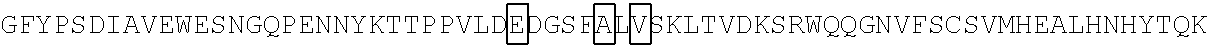

SLSLSPGK
(anti-PD-1 Ab VH-CH1-N' hinge portion-linker-IFN-a2b mutant L30A-C' hinge portion-
IgG1 Fc mutant1; VH is underlined; hinge is bolded; linker is bolded and underlined; IFN-2b
mutant is italicized)
SEQ ID NO: 109
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

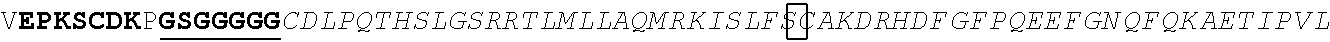

HEMIQQIFNLFSTKDSSAAWDETLLDKFYTELYQQLNDLEACVIQGVGVTETPLMKEDSILAVRKYFQRITLYLKE

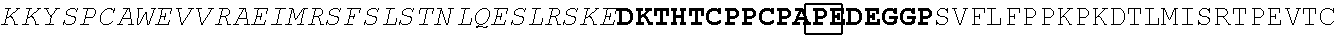

VVVDVSHEDPEVKFNWYVDGVEVHNAKTKPREEQYNSTYRVVSVLTVLHQDWLNGKEYKCKVSNKALP

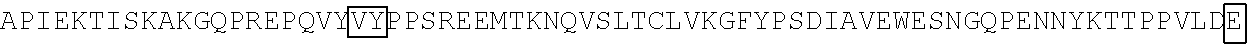

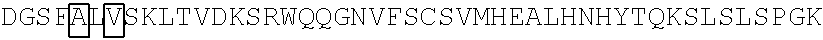

(anti-PD-1 Ab VH-CH1-N' hinge portion-linker-single-chain IFN-γ mutant A23V
homodimer-C' hinge portion-IgG1 Fc mutant1; VH is underlined; hinge is bolded; linker is
bolded and underlined; IFN-γ mutant monomer is italicized)
SEQ ID NO: 110
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

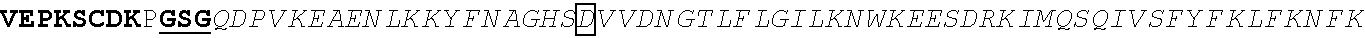

DDQSIQKSVETIKEDMNVKFFNSNKKKRDDFEKLTNYSVTDLNVORKAIHELIQVMAELSPAAKTGKRKRSQMLF

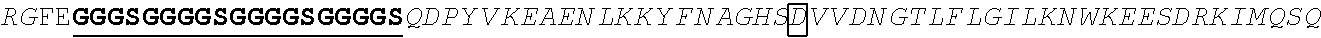

IVSFYFKLFKNFKDDOSIQKSVETIKEDMNVKFFNSNKKKRDDFEKLTNYSVTDLNVORKAIHELIQVMAELSPAA

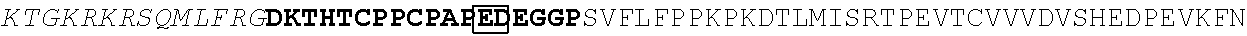

WYVDGVEVHNAKTKPREEQYNSTYRVVSVLTVLHQDWLNGKEYKCKVSNKALPAPIEKTISKAKGQPRE

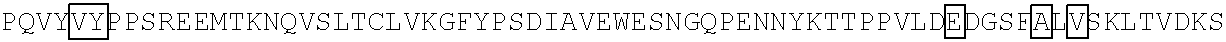

RWQQGNVFSCSVMHEALHNHYTQKSLSLSPGK
(anti-PD-1 Ab VH-CH1-N' hinge portion-linker-single-chain IL-10 mutant R27A
homodimer-C' hinge portion-IgG1 Fc mutant1; VH is underlined; hinge is bolded; linker is
bolded and underlined; IL-10 mutant monomer is italicized)
SEQ ID NO: 111
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

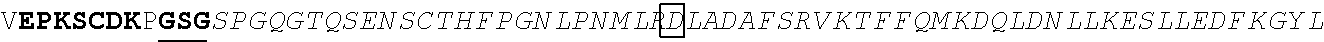

GCQALSEMIQFYLEEVMPQAENQDPDIKAHVNSLGENLKTLRLRLRRCHRFLPCENKSKAVEQVKNAFNKLQEK
GIYKAMSEFDIFINYIEAYMTMKIRNFEGGGSGGGGSGGGGSGGGGSSPGQGTQSENSCTHFPGNLPNMLRD

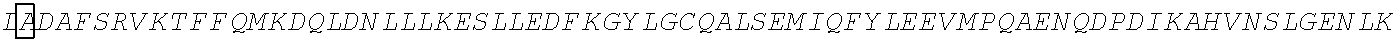

TLRLRLRRCHRFLPCENKSKAVEQVKNAFNKLQEKGIYKAMSEFDIFINYIEAYMTMKIRNDKTHTCPPCPAPE

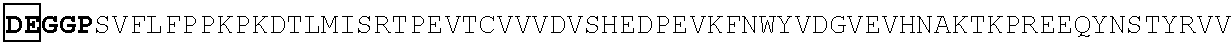

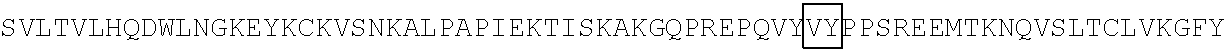

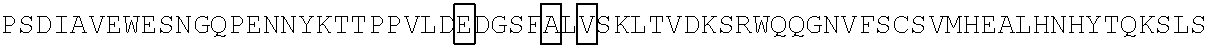

LSPGK
(anti-PD-1 Ab VH-CH1-N' hinge portion-linker-single-chain IL-12 mutant heterodimer IL-
12B (p40 E59A/F60A)-linker-IL-12A (wt p35)-C' hinge portion-IgG1 Fc mutant1; VH is underlined;
hinge is bolded; linker is bolded and underlined; IL-12 subunits are italicized)
SEQ ID NO: 112
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

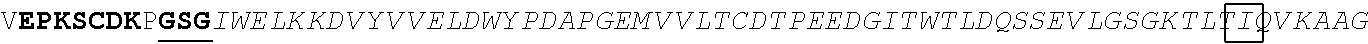

DAGQYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNFNSETVPQKSSLEEPDFYKTKIKLCILLHAFRIRAVTID

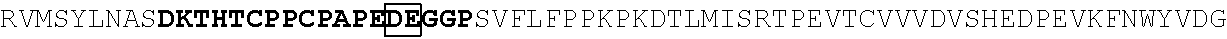

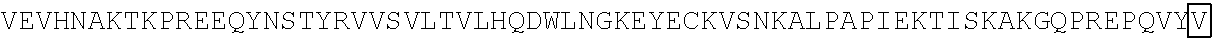

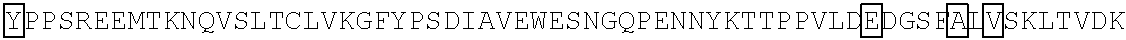

NVFSCSVMHEALHNHYTQKSLSLSPGK
(anti-PD-1 Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined;
hinge is bolded; linker is bolded and underlined; IL-23 subunits are italicized)
SEQ ID NO: 113
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

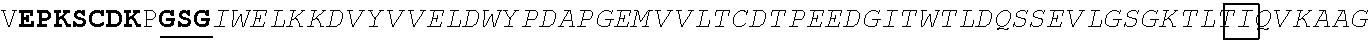

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRAVPGGSSPAWTQCQQLSQKLCTLAWSAHPLVGH
MDLREEGDEETTNDVPHIQCGDGCDPQGLRDNSQFCLORIHQGLIFYEKLLGSDIFTGEPSLLPDSPVGQLHASL
LGLSQLLQPEGHHWETQQIPSLSPSQPWQRLLLRFKILRSLQAFVAVAARVFAHGAATLSPDKTHTCPPCPAPE

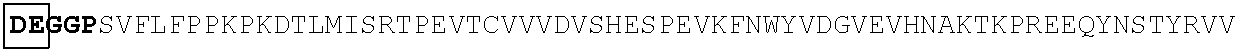

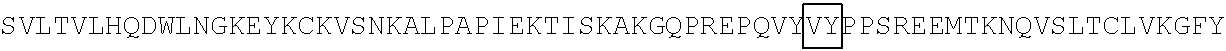

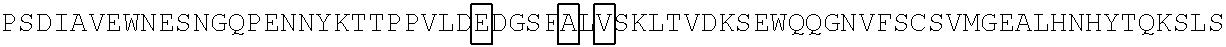

LSPGK
(G10-1 anti-CD8 Ab VH)
SEQ ID NO: 114
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTL VTVSSAS
(G10-1 anti-CD8 Ab VL)
SEQ ID NO: 115
DIQMTQTTSSLSASLGDRVTISCRASQDINNYLNWYQQKSDGTIKLLIYYTSRSYSGVPSRFSGSGSGTDYSL
TISNRRQEDIATYFCQQGKTLPWTFGGGTKLEI
(anti-CD8 Ab HC (IgG1 Fc mutant); VH is underlined; hinge is bolded)
SEQ ID NO: 116
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWY
VDGVEVHNAKTKPREEQYASTYRVVSVLTVLHQDWLNGKEYKCKVSNKALPAPIEKTISKAKGQPREPQV
YTLPPSRDELTKNQVSLTCLVKGFYPSDIA VEWESNGQPENNYKTTPPVLDSDGSFFLYSKLTVDKSRWQQ
GNVFSCSVMHEALHNHYTQKSLSLSPGK
(G10-1 anti-CD8 Ab LC; VL is underlined)
SEQ ID NO: 117
DIQMTOTTSSLSASLGDRVTISCRASQDINNYLNWYQQKSDGTIKLLIYYTSRSYSGVPSRFSGSGSGTDYSL
TISNRRQEDIATYFCQQGKTLPWTFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
(anti-CD8 Ab HC (IgG1 Fc mutant2); VH is underlined; hinge is bolded)
SEQ ID NO: 118
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

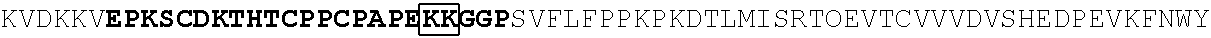

VDGVEVHNAKTKPREEQYNSTYRVVSVLTVLHQDWLNGKEYKCKVSNKALPAPIEKTISKAKGQPREPQV

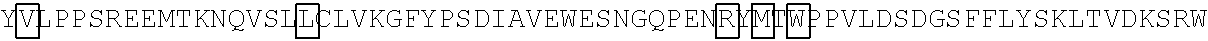

QQGNVFSCSVMHEALHNHYTQKSLSLSPGK
(anti-CD8 Ab VH-CH1-N' hinge portion-IL-2 mutant R38D/K43E/E61R-C' hinge portion-
IgG1 Fc mutant1; VH is underlined; hinge is bolded; IL-2 mutant is italicized)
SEQ ID NO: 119
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

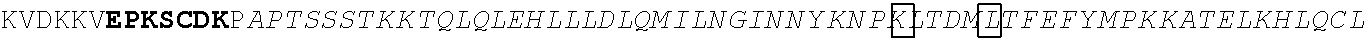

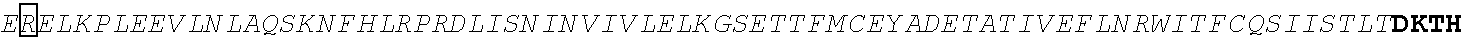

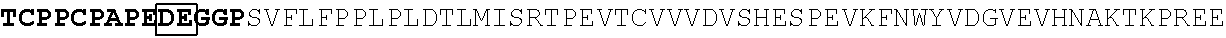

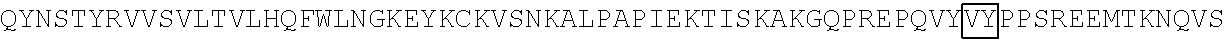

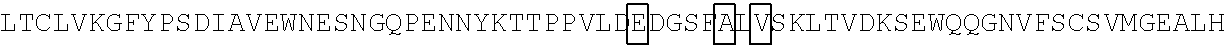

NHYTQKSLSLSPGK
(anti-CD8 Ab VH-CH1-N' hinge portion-linker-IFN-a2b mutant L30A-C' hinge portion-
IgG1 Fc mutant1; VH is underlined; hinge is bolded; linker is bolded and underlined; IFN-α2b
mutant is italicized)
SEQ ID NO: 120
EVQLQQSGPELVKPGASVKMSCKASGYTFTDYYMKWVKQSHGKSLEWIGHINPNNDDTFYTQKFKGRAT
LTVDKSSSTAYMOLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

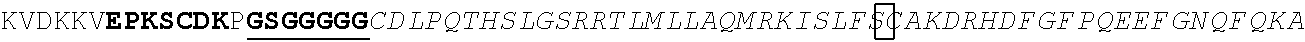

ETIPVLHEMIQQIFNLFSTKDSSAAWDETLLDKFYTELYQQLNDLEACVIQGVGVTETPLMKEDSILAVRKYFQRIT

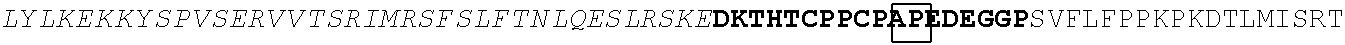

PEVTCVVVDVSHEDPEVKFNWYVDGVEVHNAKTKPREEQYNSTYRVVSVLTVLHQDWLNGKEYKCKVS

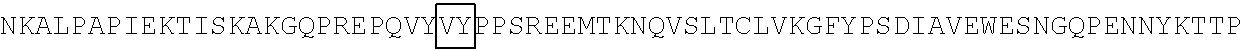

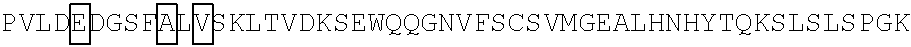

(anti-CD8 Ab VH-CH1-N' hinge portion-linker-single-chain IFN-γ mutant A23V
homodimer-C' hinge portion-IgG1 Fc mutant1; VH is underlined; hinge is bolded; linker is bolded
and underlined; IFN-γ mutant monomer is italicized)
SEQ ID NO: 121
EVQLQQSGPELVKPGASVKMSCKASGYTFTDYYMKWVKOSHGKSLEWIGHINPNNDDTFYTOKFKGRAT
LTVDKSSSTAYMOLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

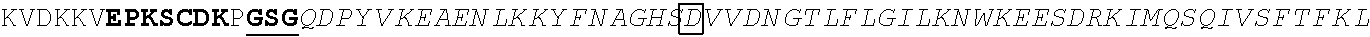

FKNFKDDQSIQKSVETIKEDMNVKFFNSNKKKRDDFEKLTNYSVTDLNVQRKAIHELIOVMAELSPAAKTGKRKR

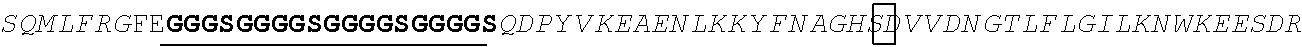

KIMQSQIVSFYFKLFKNFKDDQSIQKSVETIKEDMNVKFFNSNKKKRDDFEKLTNYSVTDLNVORKAIHELIQVMA

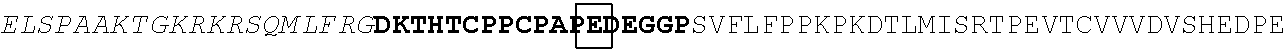

VKFNWYVDGVEVHNAKTKPREEQYNSTYRVVSVLTVLHQDWLNGKEYKCKVSNKALPAPIEKTISKAKG

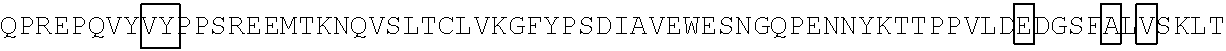

VDKSRWQQGNVFSCSVMHEALHNHYTQKSLSLSPGK
(anti-CD8 Ab VH-CH1-N' hinge portion-linker-single-chain IL-10 mutant R27A
homodimer-C' hinge portion-IgG1 Fc mutant1; VH is underlined; hinge is bolded; linker is bolded
and underlined; IL-10 mutant monomer is italicized)
SEQ ID NO: 122
EVQLQQSGPELVKPGASVKMSCKASGYTFTDYYMKWVKQSHGKSLEWIGHINPNNDDTFYTQKFKGRAT
LTVDKSSSTAYMOLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

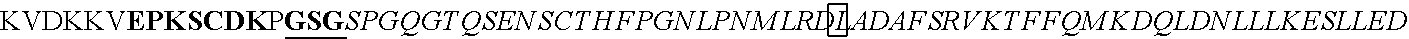

FKGYLGCQALSEMIQFYLEEVMPQAENQDPDIKAHVNSLGENLKTLRLRLRRCHRFLPCENKSKAVEQVKNAFN
KLQEKGIYKAMSEFDIFINYIEAYMTMKIRNFEGGGSGGGGSGGGGSGGGGSSPGQGTQSENSCTHFPGNLPN

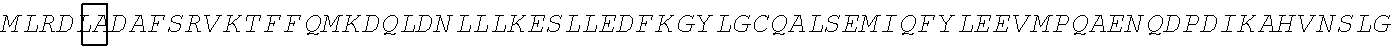

ENLKTLRLRLRRCHRFLPCENKSKAVEQVKNAFNKLOEKGIYKAMSEFDIFINYIEAYMTMKIRNDKTHTCPPCP

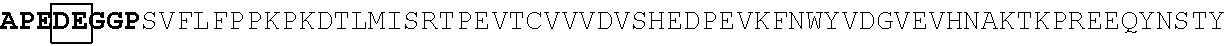

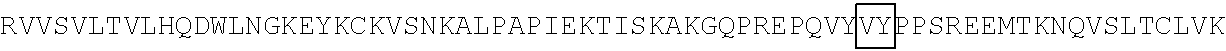

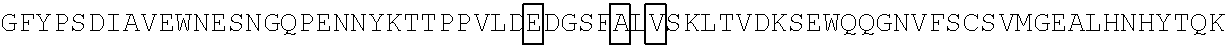

SLSLSPGK
(anti-CD8 Ab VH-CH1-N' hinge portion-linker-single-chain IL-12 mutant heterodimer IL-
12B (p40 E59A/F60A)-linker-IL-12A (wt p35)-C' hinge portion-IgG1 Fc mutant1; VH is underlined;
hinge is bolded; linker is bolded and underlined; IL-12 subunits are italicized)
SEQ ID NO: 123
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKPGSGIWELKKDVYVVELDWYPDAPGEMVVLTCDTPEEDGITWTLDQSSEVLGSGKTLTIQ

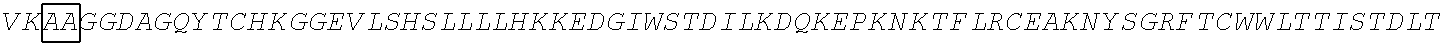

FSVKSSRGSSDPQGVTCGAATLSAERVRGDNKEYEYSVECQEDSACPAAEESLPIEVMVDAVHKLKYENYTSSFFIR
DIIKPDPPKNLOLKPLKNSROVEVSWEYPDTWSTPHSYFSLTFCVQVQGKSKREKKDRVFTDKTSATVICRKNASIS
VRAQDRYYSSSWSEWASVPCSGGGGSGGGGSGGGGSGGGGSGRNLPVATPDPGMFPCLHHSQNLLRAVSNM
LQKARQTLEFYPCTSEEIDHEDITKDKTSTVEACLPLELTKNESCLNSRETSFITNGSCLASRKTSFMMALCLSSIYE
DLKMYQVEFKTMNAKLLMDPKRQIFLDQNMLAVIDELMQALNFNSETVPQKSSLEEPDFYKTKIKLCILLHAFRI

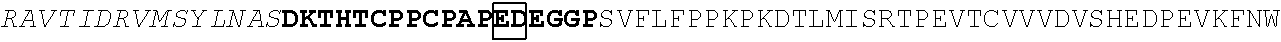

YVDGVEVHNAKTKPREEQYNSTYRVVSVLTVLHQDWLNGKEYKCKVSNKALPAPIEKTISKAKGQPREPQ

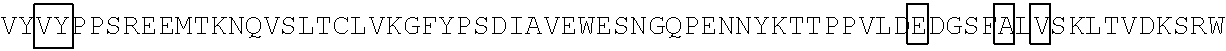

QQGNVFSCSVMHEALHNHYTQKSLSLSPGK
(anti-CD8 Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined;
hinge is bolded; linker is bolded and underlined; IL-23 subunits are italicized)
SEQ ID NO: 124
EVQLQQSGPELVKPGASVKMSCKASGYTFTDYYMKWVKQSHGKSLEWIGHINPNNDDTFYTQKFKGRAT
LTVDKSSSTAYMQLNSLTSEDSAVYYCVRDDYDGGWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKPGSGIWELKKDVYVVELDWYPDAPGEMVVLTCDTPEEDGITWTLDQSSEVLGSGKTLTIQ

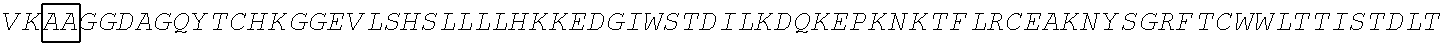

FSVKSSRGSSDPQGVTCGAATLSAERVRGDNKEYEYSVECQEDSACPAAEESLPIEVMVDAVHKLKYENYTSSFFIR
DIIKPDPPKNLQLKPLKNSRQVEVSWEYPDTWSTPHSYFSLTFCVQVQGKSKREKKDRVFTDKTSATVICRKNASIS
VRAQDRYYSSSWSEWASVPCSGGGGSGGGGSGGGGSGGGGSGRAVPGGSSPAWTQCQQLSQKLCTLAWSAH
PLVGHMDLREEGDEETTNDVPHIQCGDGCDPQGLRDNSQFCLORIHQGLIFYEKLLGSDIFTGEPSLLPDSPVG
QLHASLLGLSQLLQPEGHHWETQQIPSLSPSQPWQRLLLRFKILRSLQAFVAVAARVFAHGAATLSPDKTHTCPP

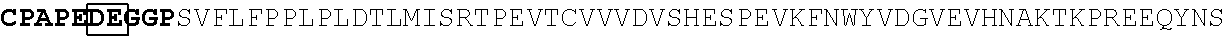

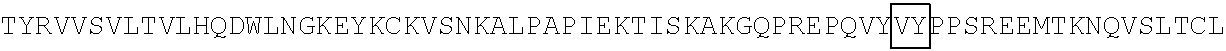

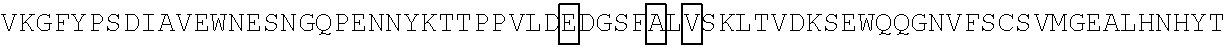

QKSLSLSPGK
(anti-CTLA-4 Ab VH)
SEQ ID NO: 125
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSAS
(ipilimumab/Yervoy anti-CTLA-4 Ab VL)
SEQ ID NO: 126
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEI
(anti-CTLA-4 Ab HC (IgG1 Fc mutant); VH is underlined; hinge is bolded)
SEQ ID NO: 127
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVD
GVEVHNAKTKPREEQYASTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGK
(ipilimumab/Yervoy anti-CTLA-4 Ab HC; VH is underlined; hinge is bolded)
SEQ ID NO: 128
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKRVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGK
(ipilimumab/Yervoy anti-CTLA-4 Ab LC; VL is underlined)
SEQ ID NO: 129
EIVLTQSPGTLSLSPGERATLSCRASQSVGSSYLAWYQQKPGQAPRLLIYGAFSRATGIPDRFSGSGSGTDFT
LTISRLEPEDFAVYYCQQYGSSPWTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
(anti-CTLA-4 Ab HC (IgG1 Fc mutant2); VH is underlined; hinge is bolded)
SEQ ID NO: 130
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV

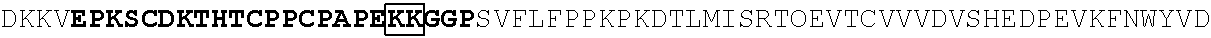

GVEVHNAKTKPREEQYNSTYRVVSVLTVLHQDWLNGKEYKCKVSNKALPAPIEKTISKAKGQPREPQVY

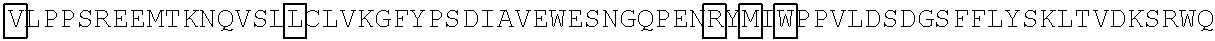

QGNVFSCSVMHEALHNHYTQKSLSLSPGK
(anti-CTLA-4 Ab VH-CH1-N' hinge portion-IL-2 mutant R38D/K43E/E61R-C' hinge
portion-IgG1 Fc mutant1; VH is underlined; hinge is bolded; IL-2 mutant is italicized)
SEQ ID NO: 131
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV

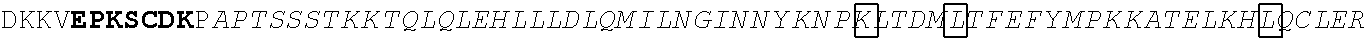

ELKPLEEVLNLAQSKNFHLRPRDLISNINVIVLELKGSETTFMCEYADETATIVEFLNRWITFCQSIISTLTDKTHTC

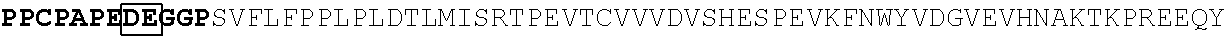

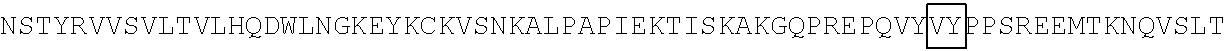

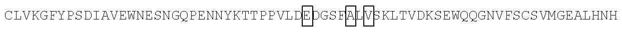

YTQKSLSLSPGK
(anti-CTLA-4 Ab VH-CH1-N' hinge portion-linker-IFN-a2b mutant L30A-C' hinge
portion-IgG1 Fc mutant1; VH is underlined; hinge is bolded; linker is bolded and underlined;
IFN-α2b mutant is italicized)
SEQ ID NO: 132
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV

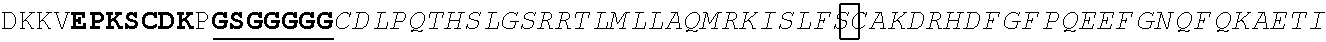

PVLHEMIQQIFNLFSTKDSSAAWDETLLDKFYTELYQQLNDLEACVIQGVGVTETPLMKEDSILAVRKYFQRITLYL

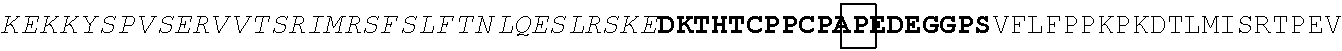

TCVVVDVSHEDPEVKFNWYVDGVEVHNAKTKPREEQYNSTYRVVSVLTVLHQDWLNGKEYKCKVSNKA

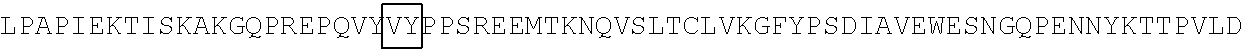

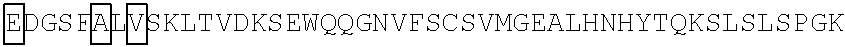

(anti-CTLA-4 Ab VH-CH1-N' hinge portion-linker-single-chain IFN-γ mutant A23V
homodimer-C' hinge portion-IgG1 Fc mutant1; VH is underlined; hinge is bolded; linker is bolded
and underlined; IFN-γ mutant monomer is italicized)
SEQ ID NO: 133
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV

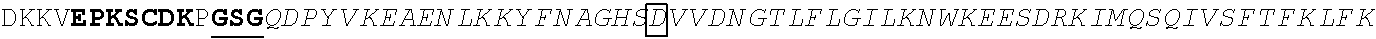

NFKDDQSIQKSVETIKEDMNVKFFNSNKKKRDDFEKLTNYSVTDLNVORKAIHELIQVMAELSPAAKTGKRKRSQ

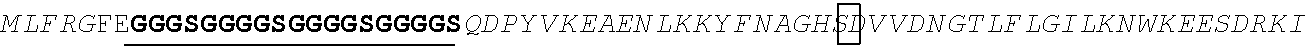

MQSQIVSFYFKLFKNFKDDQSIQKSVETIKEDMNVKFFNSNKKKRDDFEKLTNYSVTDLNVQRKAIHELIQVMAE

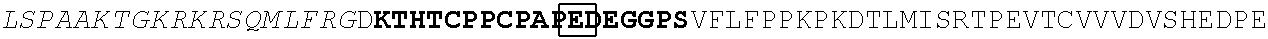

VKFNWYVDGVEVHNAKTKPREEQYNSTYRVVSVLTVLHQDWLNGKEYKCKVSNKALPAPIEKTISKAKG

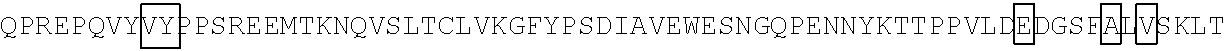

VDKSRWQQGNVFSCSVMHEALHNHYTQKSLSLSPGK
(anti-CTLA-4 Ab VH-CH1-N' hinge portion-linker-single-chain IL-10 mutant R27A
homodimer-C' hinge portion-IgG1 Fc mutant1; VH is underlined; hinge is bolded; linker is
bolded and underlined; IL-10 mutant monomer is italicized)
SEQ ID NO: 134
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV

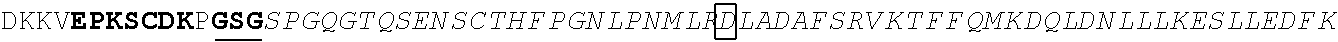

GYLGCQALSEMIOFYLEEVMPQAENODPDIKAHVNSLGENLKTLRLRLRRCHRFLPCENKSKAVEQVKNAFNKL
QEKGIYKAMSEFDIFINYIEAYMTMKIRNFEGGGSGGGGSGGGGSGGGGSSPGQGTQSENSCTHFPGNLPNM

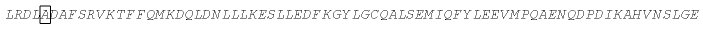

NLKTLRLRLRRCHRFLPCENKSKAVEQVKNAFNKLQEKGIYKAMSEFDIFINYIEAYMTMKIRNDKTHTCPPCPA

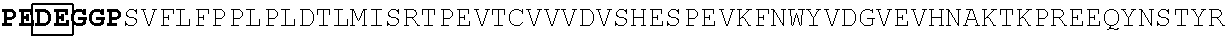

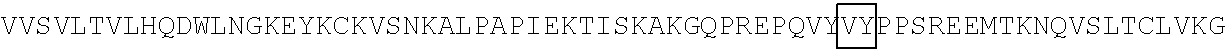

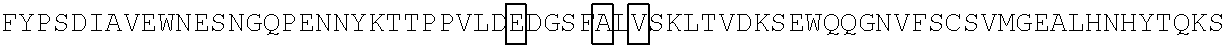

LSLSPGK
(anti-CTLA-4 Ab VH-CH1-N' hinge portion-linker-single-chain IL-12 mutant heterodimer
IL-12B (p40 E59A/F60A)-linker-IL-12A (wt p35)-C' hinge portion-IgG1 Fc mutant1; VH is
underlined; hinge is bolded; linker is bolded and underlined; IL-12 subunits are italicized)
SEQ ID NO: 135
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKKVEPKSCDKPGSGIWELKKDVYVVELDWYPDAPGEMVVLTCDTPEEDGITWTLDQSSEVLGSGKTLTIQVK

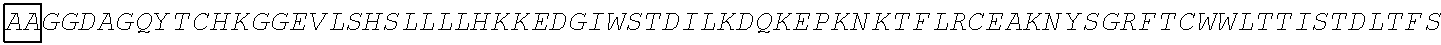

VKSSRGSSDPQGVTCGAATLSAERVRGDNKEYEYSVECQEDSACPAAEESLPIEVMVDAVHKLKYENYTSSFFIRDI
IKPDPPKNLQLKPLKNSRQVEVSWEYPDTWSTPHSYFSLTFCVQVQGKSKREKKDRVFTDKTSATVICRKNASISV
RAQDRYYSSSWSEWASVPCSGGGGSGGGGSGGGGSGGGGSGRNLPVATPDPGMFPCLHHSQNLLRAVSNML
QKARQTLEFYPCTSEEIDHEDITKDKTSTVEACLPLELTKNESCLNSRETSFITNGSCLASRKTSFMMALCLSSIYED
LKMYQVEFKTMNAKLLMDPKRQIFLDQNMLAVIDELMQALNFNSETVPQKSSLEEPDFYKTKIKLCILLHAFRIRA

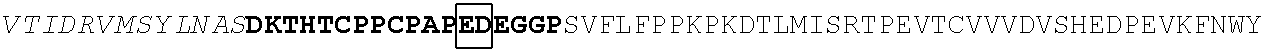

VDGVEVHNAKTKPREEQYNSTYRVVSVLTVLHQDWLNGKEYKCKVSNKALPAPIEKTISKAKGQPREPQV

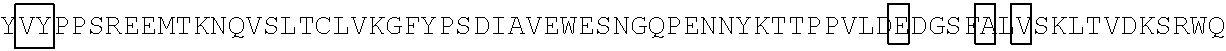

QGNVFSCSVMHEALHNHYTQKSLSLSPGK
(anti-CTLA-4 Ab VH-CH1-N' hinge portion-linker-single-chain IL-23 mutant heterodimer
IL-12B (p40 E59A/F60A)-linker-IL-23A (wt p19)-C' hinge portion-IgG1 Fc mutant1; VH is
underlined; hinge is bolded; linker is bolded and underlined; IL-23 subunits are italicized)
SEQ ID NO: 136
QVQLVESGGGVVQPGRSLRLSCAASGFTFSSYTMHWVRQAPGKGLEWVTFISYDGNNKYYADSVKGRFTI
SRDNSKNTLYLQMNSLRAEDTAIYYCARTGWLGPFDYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKV
DKKVEPKSCDKPGSGIWELKKDVYVVELDWYPDAPGEMVVLTCDTPEEDGITWTLDQSSEVLGSGKTLTIQVK

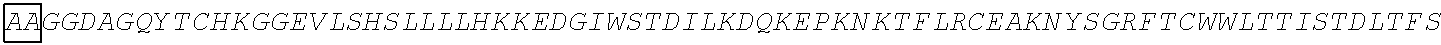

VKSSRGSSDPQGVTCGAATLSAERVRGDNKEYEYSVECQEDSACPAAEESLPIEVMVDAVHKLKYENYTSSFFIRDI
IKPDPPKNLOLKPLKNSROVEVSWEYPDTWSTPHSYFSLTFCVQVQGKSKREKKDRVFTDKTSATVICRKNASISV
RAQDRYYSSSWSEWASVPCSGGGGSGGGGSGGGGSGGGGSGRAVPGGSSPAWTQCQQLSQKLCTLAWSAHP
LVGHMDLREEGDEETTNDVPHIQCGDGCDPQGLRDNSQFCLQRIHQGLIFYEKLLGSDIFTGEPSLLPDSPVGQ
LHASLLGLSQLLQPEGHHWETQQIPSLSPSQPWQRLLLRFKILRSLQAFVAVAARVFAHGAATLSPDKTHTCPPC

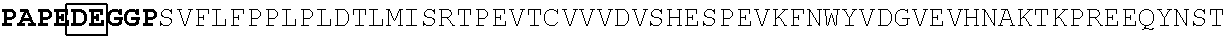

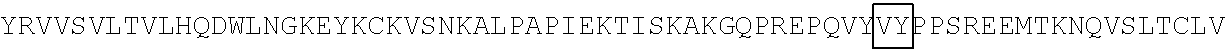

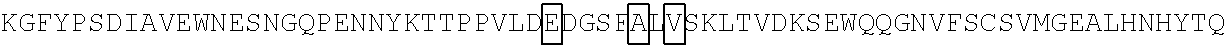

KSLSLSPGK
(anti-PD-L1 Ab HC-CDR1)
SEQ ID NO: 243
DSWIH
(anti-PD-L1 Ab HC-CDR2)
SEQ ID NO: 244
WISPYGGSSY
(anti-PD-L1 Ab HC-CDR3)
SEQ ID NO: 245
RHWPGGF
(anti-PD-L1 Ab LC-CDR1)
SEQ ID NO: 246
VTTAVA
(anti-PD-L1 Ab LC-CDR2)
SEQ ID NO: 247
SASFPY
(anti-PD-L1 Ab LC-CDR3)
SEQ ID NO: 248
QQYMYHPST
(anti-PD-L1 Ab VH; CDRs are underlined)
SEQ ID NO: 137
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSAS
(anti-PD-L1 Ab VL; CDRs are underlined)
SEQ ID NO: 138
DIQMTQSPSSLSASVGDRVTITCSASQDVTTAVAWYQQKPGKAPKLLIYSASFPYRGVPSRFSGSGSGTDFT
LTISSLQPEDFATYYCQQYMYHPSTFGQGTKVEI
(anti-PD-L1 Ab HC (IgG1 Fc mutant); VH is underlined; hinge is bolded)
SEQ ID NO: 139
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVDVSHEDPEVKFNWYV
DGVEVHNAKTKPREEQYASTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK
(anti-PD-L1 Ab LC; VL is underlined)
SEQ ID NO: 140
DIQMTQSPSSLSASVGDRVTITCSASQDVTTAVAWYQQKPGKAPKLLIYSASFPYRGVPSRFSGSGSGTDFT
LTISSLQPEDFATYYCQQYMYHPSTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
(atezolizumab/Tecentriq anti-PD-L1 Ab HC; VH is underlined; hinge is bolded)
SEQ ID NO: 141
EVQLVESGGGLVQPGGSLRLSCAASGFTFSDSWIHWVRQAPGKGLEWVAWISPYGGSTYYADSVKGRFTI
SADTSKNTAYLQMNSLRAEDTAVYYCARRHWPGGFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVDVSHEDPEVKFNWYV
DGVEVHNAKTKPREEQYASTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
(atezolizumab/Tecentriq anti-PD-L1 Ab LC; VL is underlined)
SEQ ID NO: 142
DIQMTQSPSSLSASVGDRVTITCRASQDVSTAVAWYQQKPGKAPKLLIYSASFLYSGVPSRFSGSGSGTDFT
LTISSLQPEDFATYYCQQYLYHPATFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
(anti-PD-L1 Ab HC (IgG1 Fc mutant2); VH is underlined; hinge is bolded)
SEQ ID NO: 143
EVQLVESGGGLVQPGGSLRLSCAASYFPFSDSWIHWVRQAPGKGLEWVAWISPYGGSSYYADSVODRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK

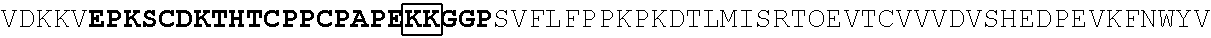

DGVEVHNAKTKPREEQYNSTYRVVSVLTVLHQDWLNGKEYKCKVSNKALPAPIEKTISKAKGQPREPQVY

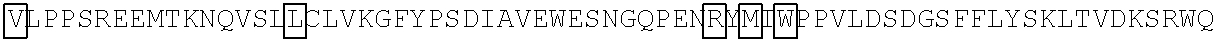

DWPPVLDSDGSFFLYSKLTVDKSRWQ
QGNVFSCSVMHEALHNHYTQKSLSLSPGK
(anti-PD-L1 Ab VH-CH1-N' hinge portion-IL-2 mutant R38D/K43E/E61R-C' hinge
portion-IgG1 Fc mutant1; VH is underlined; hinge is bolded; IL-2 mutant is italicized)
SEQ ID NO: 144
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK

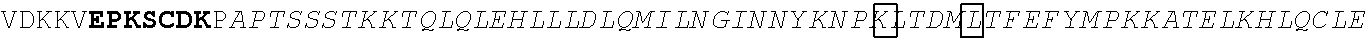

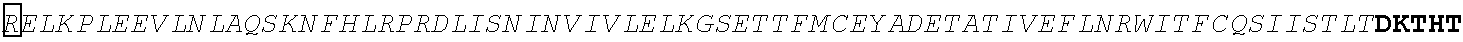

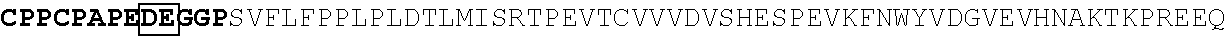

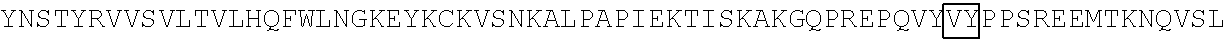

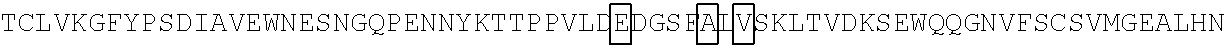

HYTQKSLSLSPGK
(anti-PD-L1 Ab VH-CH1-N' hinge portion-linker-IFN-a2b mutant L30A-C' hinge portion-
IgG1 Fc mutant1; VH is underlined; hinge is bolded; linker is bolded and underlined; IFN-α2b
mutant is italicized)
SEQ ID NO: 145
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK

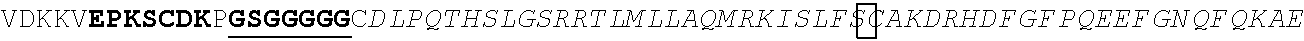

TIPVLHEMIQQIFNLFSTKDSSAAWDETLLDKFYTELYQQLNDLEACVIQGVGVTETPLMKEDSILAVRKYFQRITL

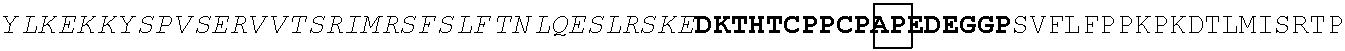

EVTCVVVDVSHEDPEVKFNWYVDGVEVHNAKTKPREEQYNSTYRVVSVLTVLHQDWLNGKEYKCKVSN

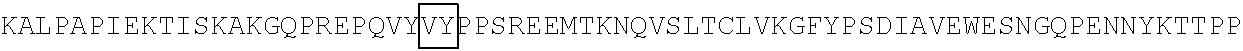

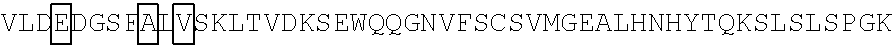

(anti-PD-L1 Ab VH-CH1-N' hinge portion-linker-single-chain IFN-y mutant A23V
homodimer-C' hinge portion-IgG1 Fc mutant1; VH is underlined; hinge is bolded; linker is bolded
and underlined; IFN-γ mutant monomer is italicized)
SEQ ID NO: 146
EVQLVESGGGLVQPGGSLRLSCAASYFPFSDSWIHWVRQAPGKGLEWVAWISPYGGSSYYADSVODRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK

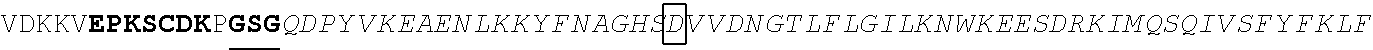

KNFKDDOSIOKSVETIKEDMNVKFFNSNKKKRDDFEKLTNYSVTDLNVORKAIHELIOVMAELSPAAKTGKRKRS

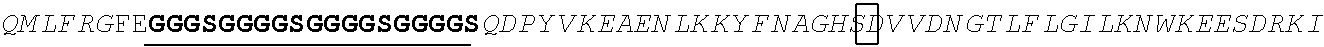

MQSQIVSFYFKLFKNFKDDOSIQKSVETIKEDMNVKFFNSNKKKRDDFEKLTNYSVTDLNVORKAIHELIQVMAE

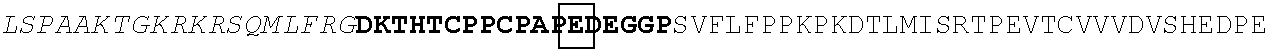

VKFNWYVDGVEVHNAKTKPREEQYNSTYRVVSVLTVLHQDWLNGKEYKCKVSNKALPAPIEKTISKAKG

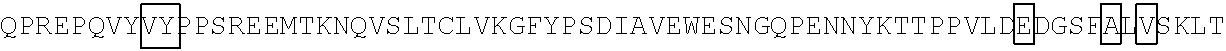

VDKSRWQQGNVFSCSVMHEALHNHYTQKSLSLSPGK
(anti-PD-L1 Ab VH-CH1-N' hinge portion-linker-single-chain IL-10 mutant R27A
homodimer-C' hinge portion-IgG1 Fc mutant1; VH is underlined; hinge is bolded; linker is
bolded and underlined; IL-10 mutant monomer is italicized)
SEQ ID NO: 147
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK

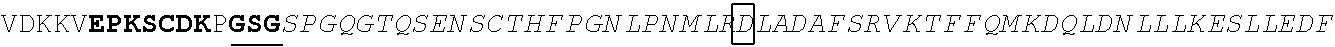

KGYLGCQALSEMIQFYLEEVMPQAENQDPDIKAHVNSLGENLKTLRLRLRRCHRFLPCENKSKAVEQVKNAFNK
LQEKGIYKAMSEFDIFINYIEAYMTMKIRNFEGGGSGGGGSGGGGSGGGGSSPGQGTQSENSCTHFPGNLPN

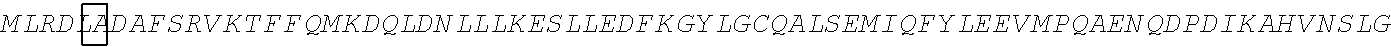

ENLKTLRLRLRRCHRFLPCENKSKAVEQVKNAFNKLQEKGIYKAMSEFDIFINYIEAYMTMKIRNDKTHTCPPCP

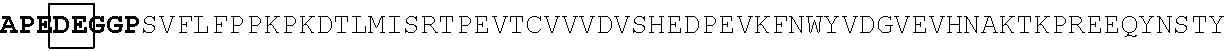

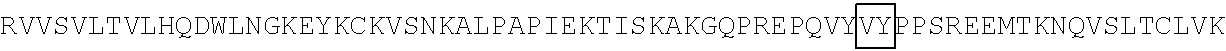

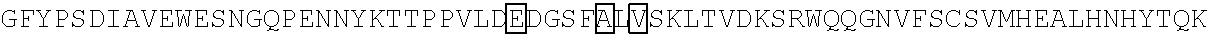

SLSLSPGK
(anti-PD-L1 Ab VH-CH1-N' hinge portion-linker-single-chain IL-12 mutant heterodimer
IL-12B (p40 E59A/F60A)-linker-IL-12A (wt p35)-C' hinge portion-IgG1 Fc mutant1; VH is
underlined; hinge is bolded; linker is bolded and underlined; IL-12 subunits are italicized)
SEQ ID NO: 148
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK
VDKKVEPKSCDKPGSGIWELKKDVYVVELDWYPDAPGEMVVLTCDTPEEDGITWTLDOSSEVLGSGKTLTIQV

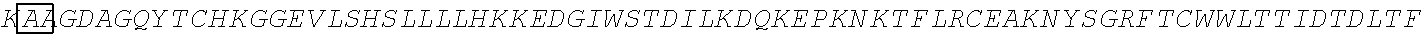

SVKSSRGSSDPQGVTCGAATLSAERVRGDNKEYEYSVECQEDSACPAAEESLPIEVMVDAVHKLKYENYTSSFFIRD
IIKPDPPKNLOLKPLKNSRQVEVSWEYPDTWSTPHSYFSLTFCVQVQGKSKREKKDRVFTDKTSATVICRKNASISV
RAQDRYYSSSWSEWASVPCSGGGGSGGGGSGGGGSGGGGSGRNLPVATPDPGMFPCLHHSONLLRAVSNML
QKARQTLEFYPCTSEEIDHEDITKDKTSTVEACLPLELTKNESCLNSRETSFITNGSCLASRKTSFMMALCLSSIYED
LKMYQVEFKTMNAKLLMDPKRQIFLDQNMLAVIDELMQALNFNSETVPQKSSLEEPDFYKTKIKLCILLHAFRIRA

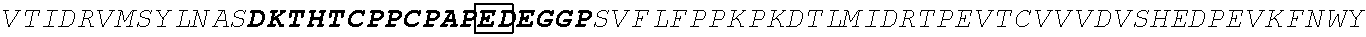

VDGVEVHNAKTKPREEQYNSTYRVVSVLTVLHQDWLNGKEYKCKVSNKALPAPIEKTISKAKGQPREPQV

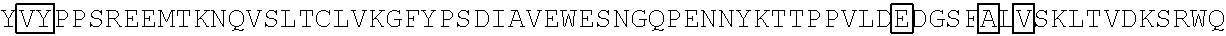

QGNVFSCSVMHEALHNHYTQKSLSLSPGK
(anti-PD-L1 Ab VH-CH1-N' hinge portion-linker-single-chain IL-23 mutant heterodimer
IL-12B (p40 E59A/F60A)-linker-IL-23A (wt p19)-C' hinge portion-IgG1 Fc mutant1; VH is
underlined; hinge is bolded; linker is bolded and underlined; IL-23 subunits are italicized)
SEQ ID NO: 149
EVQLVESGGGLVQPGGSLRLSCAASYFPFSDSWIHWVRQAPGKGLEWVAWISPYGGSSYYADSVQDRFTIS
ADTSKNTAYLQMNSLRAEDTAVYYCAKRHWPGGFDHWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTK
VDKKVEPKSCDKPGSGIWELKKDVYVVELDWYPDAPGEMVVLTCDTPEEDGITWTLDQSSEVLGSGKTLTIQV

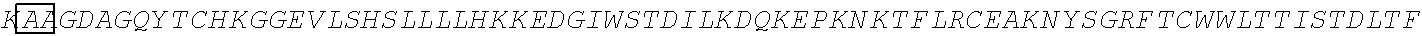

SVKSSRGSSDPQGVTCGAATLSAERVRGDNKEYEYSVECQEDSACPAAEESLPIEVMVDAVHKLKYENYTSSFFIRD
IIKPDPPKNLOLKPLKNSRQVEVSWEYPDTWSTPHSYFSLTFCVQVQGKSKREKKDRVFTDKTSATVICRKNASISV
RAQDRYYSSSWSEWASVPCSGGGGSGGGGSGGGGSGGGGSGRAVPGGSSPAWTQCQQLSQKLCTLAWSAHP
LVGHMDLREEGDEETTNDVPHIQCGDGCDPQGLRDNSQFCLQRIHQGLIFYEKLLGSDIFTGEPSLLPDSPVGQ
LHASLLGLSQLLQPEGHHWETQQIPSLSPSQPWQRLLLRFKILRSLQAFVAVAARVFAHGAATLSPDKTHTCPPC

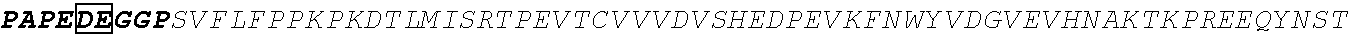

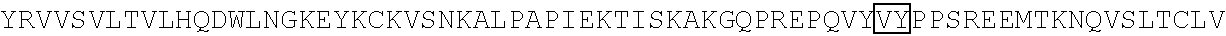

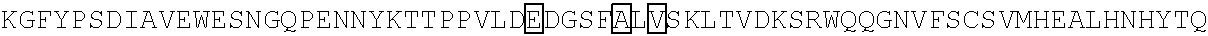

KSLSLSPGK
(trastuzumab/Herceptin anti-HER2 Ab HC-CDR1)
SEQ ID NO: 188
GFNIKDTYIH
(trastuzumab/Herceptin anti-HER2 Ab HC-CDR2)
SEQ ID NO: 189
RIYPTNGYTRYADSVKG
(trastuzumab/Herceptin anti-HER2 Ab HC-CDR3)
SEQ ID NO: 190
WGGDGFYAMDY
(trastuzumab/Herceptin anti-HER2 Ab LC-CDR1)
SEQ ID NO: 191
RASQDVNTAVA
(trastuzumab/Herceptin anti-HER2 Ab LC-CDR2)
SEQ ID NO: 192
SASFLYS
(trastuzumab/Herceptin anti-HER2 Ab LC-CDR3)
SEQ ID NO: 193
QQHYTTPPT
(trastuzumab/Herceptin anti-HER2 Ab VH)
SEQ ID NO: 150
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSAS
(trastuzumab/Herceptin anti-HER2 Ab VL)
SEQ ID NO: 151
DIQMTQSPSSLSASVGDRVTITCRASQDVNTAVAWYQQKPGKAPKLLIYSASFLYSGVPSRFSGSRSGTDFT
LTISSLQPEDFATYYCQQHYTTPPTFGQGTK VEI
(anti-HER2 Ab HC (IgG1 Fc mutant); VH is underlined; hinge is bolded)
SEQ ID NO: 152
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWY
VDGVEVHNAKTKPREEQYASTYRVVSVLTVLHQDWLNGKEYKCKVSNKALPAPIEKTISKAKGQPREPQV
YTLPPSRDELTKNQVSLTCLVKGFYPSDIA VEWESNGQPENNYKTTPPVLDSDGSFFLYSKLTVDKSRWQQ
GNVFSCSVMHEALHNHYTQKSLSLSPGK
(trastuzumab/Herceptin anti-HER2 Ab HC; VH is underlined; hinge is bolded)
SEQ ID NO: 153
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
(trastuzumab/Herceptin anti-HER2 Ab LC; VL is underlined)
SEQ ID NO: 154
DIQMTQSPSSLSASVGDRVTITCRASQDVNTAVAWYQQKPGKAPKLLIYSASFLYSGVPSRFSGSRSGTDFT
LTISSLQPEDFATYYCQQHYTTPPTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
(anti-HER2 Ab HC (IgG1 Fc mutant2); VH is underlined; hinge is bolded)
SEQ ID NO: 155
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

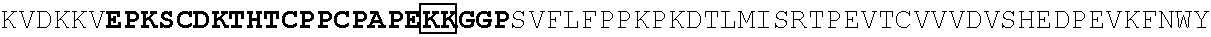

VDGVEVHNAKTKPREEQYNSTYRVVSVLTVLHQDWLNGKEYKCKVSNKALPAPIEKTISKAKGQPREPQV

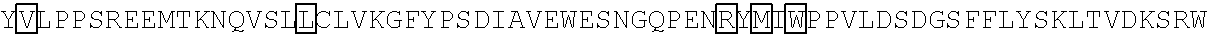

QQGNVFSCSVMHEALHNHYTQKSLSLSPGK
(anti-HER2 Ab VH-CH1-N' hinge portion-IL-2 mutant R38D/K43E/E61R-C' hinge
portion-IgG1 Fc mutant1; VH is underlined; hinge is bolded; IL-2 mutant is italicized)
SEQ ID NO: 156
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

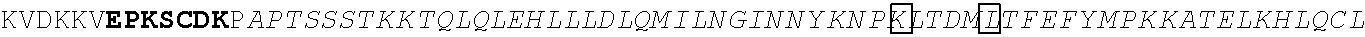

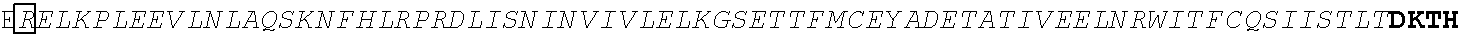

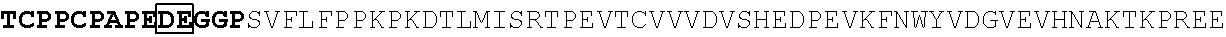

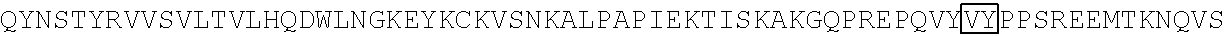

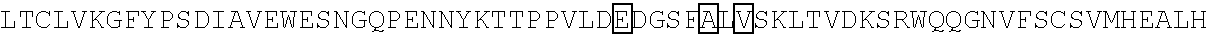

NHYTQKSLSLSPGK
(anti-HER2 Ab VH-CH1-N' hinge portion-linker-IFN-α2b mutant L30A-C' hinge portion-
IgG1 Fc mutant1; VH is underlined; hinge is bolded; linker is bolded and underlined;
IFN-α2b mutant is italicized)
SEQ ID NO: 157
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

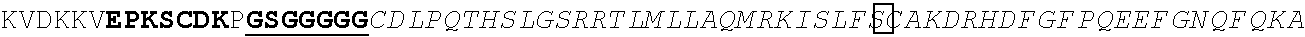

ETIPVLHEMIQQIFNLFSTKDSSAAWDETLLDKFYTELYQQLNDLEACVIQGVGVTETPLMKEDSILAVRKYFQRIT

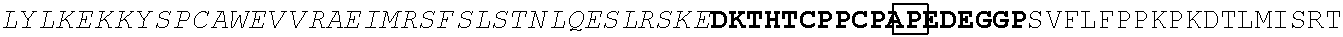

PEVTCVVVDVSHEDPEVKFNWYVDGVEVHNAKTKPREEQYNSTYRVVSVLTVLHQDWLNGKEYKCKVS

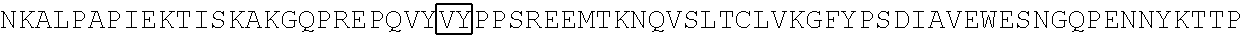

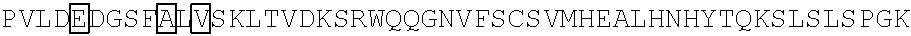

(anti-HER2 Ab VH-CH1-N' hinge portion-linker-single-chain IFN-γ mutant A23V
homodimer-C' hinge portion-IgG1 Fc mutant1; VH is underlined; hinge is bolded; linker is bolded
and underlined; IFN-γ mutant monomer is italicized)
SEQ ID NO: 158
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

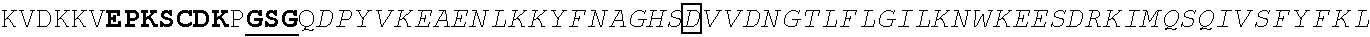

FKNFKDDQSIQKSVETIKEDMNVKFFNSNKKKRDDFEKLTNYSVTDLNVORKAIHELIQVMAELSPAAKTGKRKR

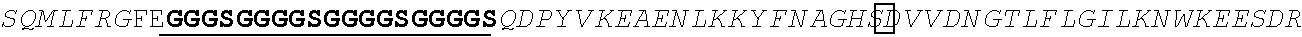

KIMQSQIVSFYFKLFKNFKDDOSIOKSVETIKEDMNVKFFNSNKKKRDDFEKLTNYSVTDLNVQRKAIHELIQVMA

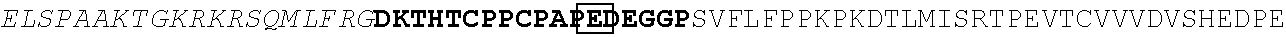

VKFNWYVDGVEVHNAKTKPREEQYNSTYRVVSVLTVLHQDWLNGKEYKCKVSNKALPAPIEKTISKAKG

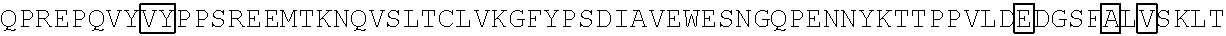

VDKSRWQQGNVFSCSVMHEALHNHYTQKSLSLSPGK
(anti-HER2 Ab VH-CH1-N' hinge portion-linker-single-chain IL-10 mutant R27A
homodimer-C' hinge portion-IgG1 Fc mutant1; VH is underlined; hinge is bolded; linker is
bolded and underlined; IL-10 mutant monomer is italicized)
SEQ ID NO: 159
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT

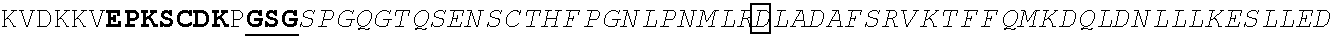

FKGYLGCQALSEMIQFYLEEVMPQAENQDPDIKAHVNSLGENLKTLRLRLRRCHRFLPCENKSKAVEQVKNAFN
KLQEKGIYKAMSEFDIFINYIEAYMTMKIRNFEGGGSGGGGSGGGGSGGGGSSPGQGTQSENSCTHFPGNLPN

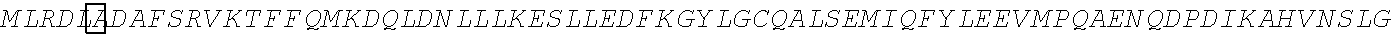

ENLKTLRLRLRRCHRFLPCENKSKAVEQVKNAFNKLQEKGIYKAMSEFDIFINYIEAYMTMKIRNDKTHTCPPCP

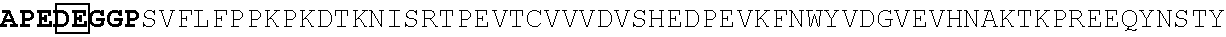

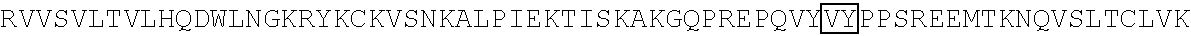

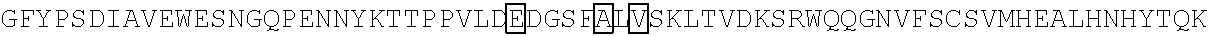

SLSLSPGK
(anti-HER2 Ab VH-CH1-N' hinge portion-linker-single-chain IL-12 mutant heterodimer
IL-12B (p40 E59A/F60A)-linker-IL-12A (wt p35)-C' hinge portion-IgG1 Fc mutant1; VH is
underlined; hinge is bolded; linker is bolded and underlined; IL-12 subunits are italicized)
SEQ ID NO: 160
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKPGSGIWELKKDVYVVELDWYPDAPGEMVVLTCDTPEEDGITWTLDQSSEVLGSGKTLTIQ

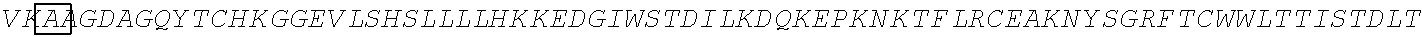

FSVKSSRGSSDPQGVTCGAATLSAERVRGDNKEYEYSVECQEDSACPAAEESLPIEVMVDAVHKLKYENYTSSFFIR
DIIKPDPPKNLOLKPLKNSRQVEVSWEYPDTWSTPHSYFSLTFCVQVQGKSKREKKDRVFTDKTSATVICRKNASIS
VRAQDRYYSSSWSEWASVPCSGGGGSGGGGSGGGGSGGGGSGRNLPVATPDPGMFPCLHHSQNLLRAVSNM
LQKARQTLEFYPCTSEEIDHEDITKDKTSTVEACLPLELTKNESCLNSRETSFITNGSCLASRKTSFMMALCLSSIYE
DLKMYQVEFKTMNAKLLMDPKRQIFLDQNMLAVIDELMQALNFNSETVPQKSSLEEPDFYKTKIKLCILLHAFRI

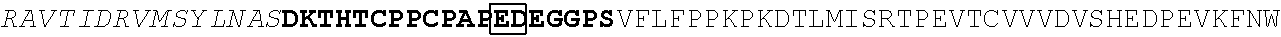

YVDGVEVHNAKTKPREEQYNSTYRVVSVLTVLHQDWLNGKEYKCKVSNKALPAPIEKTISKAKGQPREPQ

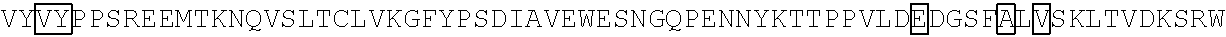

QQGNVFSCSVMHEALHNHYTQKSLSLSPGK
(anti-HER2 Ab VH-CH1-N' hinge portion-linker-single-chain IL-23 mutant heterodimer
IL-12B (p40 E59A/F60A)-linker-IL-23A (wt p19)-C' hinge portion-IgG1 Fc mutant1; VH is
underlined; hinge is bolded; linker is bolded and underlined; IL-23 subunits are italicized)
SEQ ID NO: 161
EVQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSNT
KVDKKVEPKSCDKPGSGIWELKKDVYVVELDWYPDAPGEMVVLTCDTPEEDGITWTLDQSSEVLGSGKTLTIQ

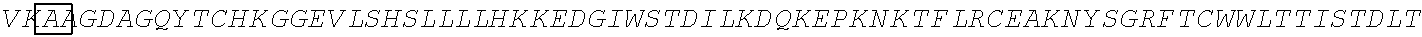

FSVKSSRGSSDPQGVTCGAATLSAERVRGDNKEYEYSVECQEDSACPAAEESLPIEVMVDAVHKLKYENYTSSFFIR
DIIKPDPPKNLQLKPLKNSRQVEVSWEYPDTWSTPHSYFSLTFCVQVQGKSKREKKDRVFTDKTSATVICRKNASIS
VRAQDRYYSSSWSEWASVPCSGGGGSGGGGSGGGGSGGGGSGRAVPGGSSPAWTQCQQLSQKLCTLAWSAH
PLVGHMDLREEGDEETTNDVPHIQCGDGCDPQGLRDNSQFCLORIHQGLIFYEKLLGSDIFTGEPSLLPDSPVG
QLHASLLGLSQLLQPEGHHWETQQIPSLSPSQPWQRLLLRFKILRSLQAFVAVAARVFAHGAATLSPDKTHTCPP

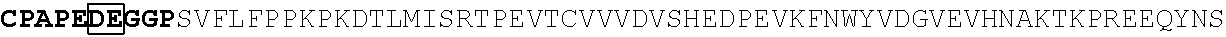

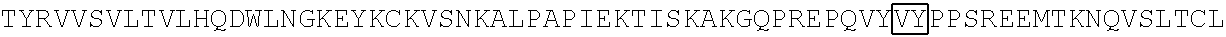

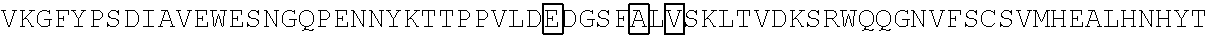

QKSLSLSPGK
(basiliximab/Simulect anti-CD25 Ab VH)
SEQ ID NO: 162
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGQGTTLTVSSAS
(basiliximab/Simulect anti-CD25 Ab VL)
SEQ ID NO: 163
QIVSTQSPAIMSASPGEKVTMTCSASSSRSYMQWYQQKPGTSPKRWIYDTSKLASGVPARFSGSGSGTSYSL
TISSMEAEDAATYYCHQRSSYTFGGGTKLEI
(anti-CD25 Ab HC (IgG1 Fc mutant); VH is underlined; hinge is bolded)
SEQ ID NO: 164
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQGNVFS
CSVMHEALHNHYTQKSLSLSPGK
(basiliximab/Simulect anti-CD25 Ab LC; VL is underlined)
SEQ ID NO: 165
QIVSTQSPAIMSASPGEKVTMTCSASSSRSYMQWYQQKPGTSPKRWIYDTSKLASGVPARFSGSGSGTSYSL
TISSMEAEDAATYYCHQRSSYTFGGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
(basiliximab/Simulect anti-CD25 Ab HC; VH is underlined; hinge is bolded)
SEQ ID NO: 166
QLQQSGTVLARPGASVKMSCKASGYSFTRYWMHWIKQRPGQGLEWIGAIYPGNSDTSYNOKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK
RVEPPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVF
SCSVMHEALHNHYTQKSLSLSPGK
(basiliximab/Simulect anti-CD25 Ab LC; VL is underlined)
SEQ ID NO: 167
QIVSTQSPAIMSASPGEK VTMTCSASSSRSYMQWYQQKPGTSPKRWIYDTSKLASGVPARFSGSGSGTSYSL
TISSMEAEDAATYYCHQRSSYTFGGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGE
(anti-CD25 Ab HC (IgG1 Fc mutant2); VH is underlined; hinge is bolded)
SEQ ID NO: 168
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGOGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

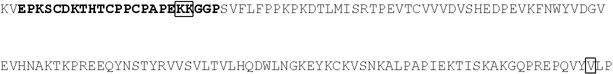

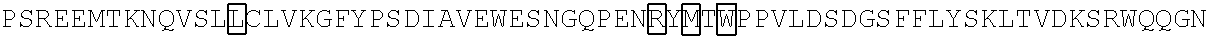

VFSCSVMHEALHNHYTQKSLSLSPGK
(anti-CD25 Ab VH-CH1-N' hinge portion-IL-2 mutant R38D/K43E/E61R-C' hinge portion-
IgG1 Fc mutant1; VH is underlined; hinge is bolded; IL-2 mutant is italicized)
SEQ ID NO: 169
QLQQSGTVLARPGASVKMSCKASGYSFTRYWMHWIKQRPGQGLEWIGAIYPGNSDTSYNOKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

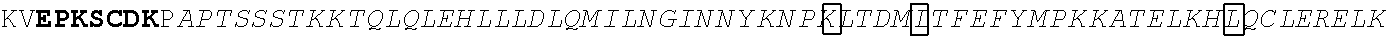

PLEEVLNLAOSKNFHLRPRDLISNINVIVLELKGSETTFMCEYADETATIVEFLNRWITFCOSIISTLTDKTHTCPPC

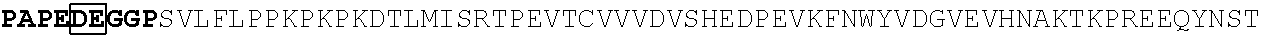

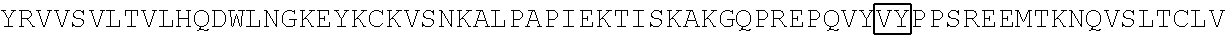

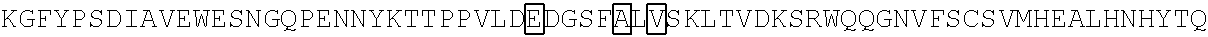

KSLSLSPGK
(anti-CD25 Ab VH-CH1-N' hinge portion-linker-IFN-a2b mutant L30A-C' hinge portion-
IgG1 Fc mutant1; VH is underlined; hinge is bolded; linker is bolded and underlined;
IFN-α2b mutant is italicized)
SEQ ID NO: 170
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGOGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

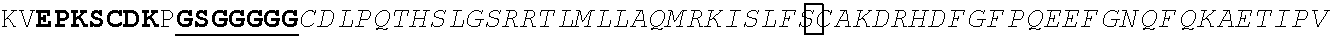

LHEMIQQIFNLFSTKDSSAAWDETLLDKFYTELYQQLNDLEACVIQGVGVTETPLMKEDSILAVRKYFORITLYLKE

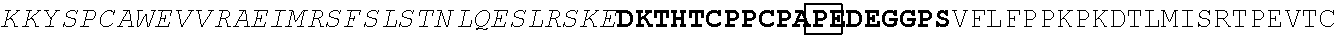

VVVDVSHEDPEVKFNWYVDGVEVHNAKTKPREEQYNSTYRVVSVLTVLHQDWLNGKEYKCKVSNKALP

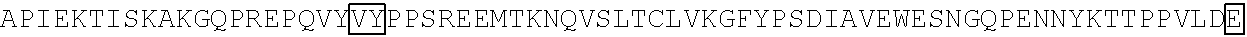

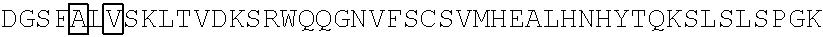

(anti-CD25 Ab VH-CH1-N' hinge portion-linker-single-chain IFN-γ mutant A23V
homodimer-C' hinge portion-IgG1 Fc mutant1; VH is underlined; hinge is bolded; linker is
bolded and underlined; IFN-γ mutant monomer is italicized)
SEQ ID NO: 171
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

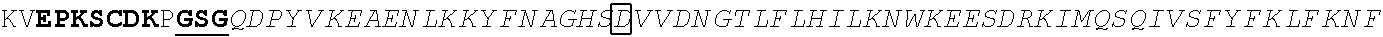

KDDQSIOKSVETIKEDMNVKFFNSNKKKRDDFEKLTNYSVTDLNVORKAIHELIQVMAELSPAAKTGKRKRSQML

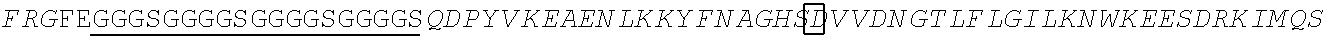

QIVSFYFKLFKNFKDDQSIQKSVETIKEDMNVKFFNSNKKKRDDFEKLTNYSVTDLNVORKAIHELIQVMAELSPA

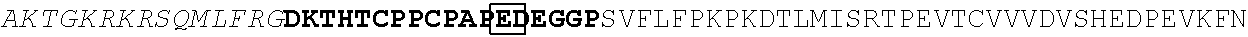

WYVDGVEVHNAKTKPREEQYNSTYRVVSVLTVLHQDWLNGKEYKCKVSNKALPAPIEKTISKAKGQPRE

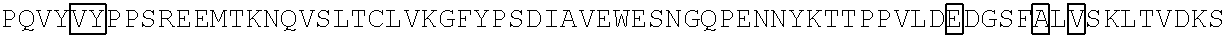

RWQQGNVFSCSVMHEALHNHYTQKSLSLSPGK
(anti-CD25 Ab VH-CH1-N' hinge portion-linker-single-chain IL-10 mutant R27A
homodimer-C' hinge portion-IgG1 Fc mutant1; VH is underlined; hinge is bolded; linker is
bolded and underlined; IL-10 mutant monomer is italicized)
SEQ ID NO: 172
QLQQSGTVLARPGASVKMSCKASGYSFTRYWMHWIKQRPGQGLEWIGAIYPGNSDTSYNOKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

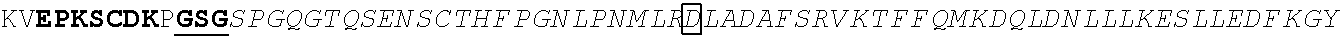

LGCQALSEMIQFYLEEVMPQAENQDPDIKAHVNSLGENLKTLRLRLRRCHRFLPCENKSKAVEQVKNAFNKLQE
KGIYKAMSEFDIFINYIEAYMTMKIRNFEGGGSGGGGSGGGGSGGGGSSPGQGTQSENSCTHFPGNLPNMLR

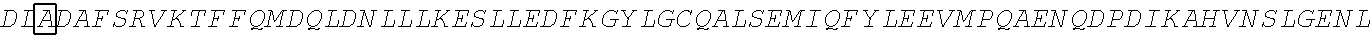

KTLRLRLRRCHRFLPCENKSKAVEQVKNAFNKLQEKGIYKAMSEFDIFINYIEAYMTMKIRNDKTHTCPPCPAPE

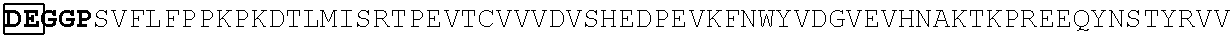

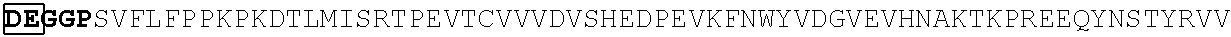

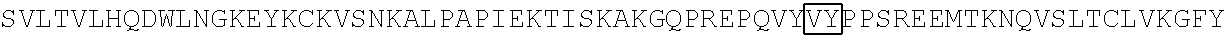

LSPGK
(anti-CD25 Ab VH-CH1-N' hinge portion-linker-single-chain IL-12 mutant heterodimer IL-
12B (p40 E59A/F60A)-linker-IL-12A (wt p35)-C' hinge portion-IgG1 Fc mutant1; VH is underlined;
hinge is bolded; linker is bolded and underlined; IL-12 subunits are italicized)
SEQ ID NO: 173
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

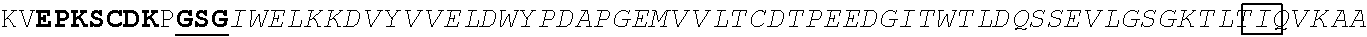

GDAGQYTCHKGGEVLSHSLLLLHKKEDGIWSTDILKDOKEPKNKTFLRCEAKNYSGRFTCWWLTTISTDLTFSVK
SSRGSSDPQGVTCGAATLSAERVRGDNKEYEYSVECOEDSACPAAEESLPIEVMVDAVHKLKYENYTSSFFIRDIIK
PDPPKNLOLKPLKNSROVEVSWEYPDTWSTPHSYFSLTFCVOVOGKSKREKKDRVFTDKTSATVICRKNASISVRA
QDRYYSSSWSEWASVPCSGGGGSGGGGSGGGGSGGGGSGRNLPVATPDPGMFPCLHHSQNLLRAVSNMLQK
ARQTLEFYPCTSEEIDHEDITKDKTSTVEACLPLELTKNESCLNSRETSFITNGSCLASRKTSFMMALCLSSIYEDLK
MYQVEFKTMNAKLLMDPKROIFLDONMLAVIDELMQALNFNSETVPQKSSLEEPDFYKTKIKLCILLHAFRIRAVT

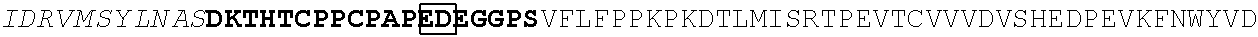

GVEVHNAKTKPREEQYNSTYRVVSVLTVLHQDWLNGKEYKCKVSNKALPAPIEKTISKAKGQPREPQVY

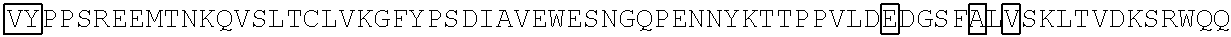

GNVFSCSVMHEALHNHYTQKSLSLSPGK
(anti-CD25 Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined;
hinge is bolded; linker is bolded and underlined; IL-23 subunits are italicized)
SEQ ID NO: 174
QLQQSGTVLARPGASVKMSCKASGYSFTRYWMHWIKQRPGQGLEWIGAIYPGNSDTSYNQKFEGKAKLT
AVTSASTAYMELSSLTHEDSAVYYCSRDYGYYFDFWGQGTTLTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDK

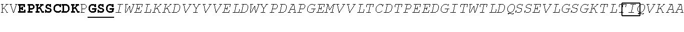

GDAGQYTCHKGGEVLSHSLLLLHKKEDGIWSTDILKDOKEPKNKTFLRCEAKNYSGRFTCWWLTTISTDLTFSVK
SSRGSSDPQGVTCGAATLSAERVRGDNKEYEYSVECQEDSACPAAEESLPIEVMVDAVHKLKYENYTSSFFIRDIIK
PDPPKNLOLKPLKNSRQVEVSWEYPDTWSTPHSYFSLTFCVQVQGKSKREKKDRVFTDKTSATVICRKNASISVRA
QDRYYSSSWSEWASVPCSGGGGSGGGGSGGGGSGGGGSGRAVPGGSSPAWTQCQQLSQKLCTLAWSAHPLV
GHMDLREEGDEETTNDVPHIQCGDGCDPQGLRDNSQFCLQRIHQGLIFYEKLLGSDIFTGEPSLLPDSPVGQLH
ASLLGLSQLLQPEGHHWETQQIPSLSPSQPWQRLLLRFKILRSLQAFVAVAARVFAHGAATLSPDKTHTCPPCPA

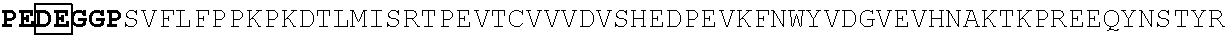

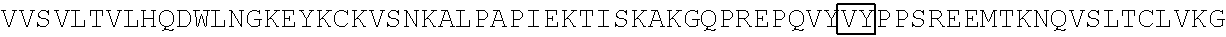

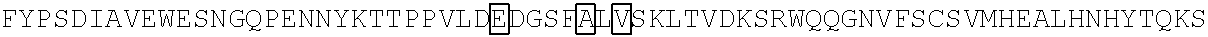

LSLSPGK
(wildtype human PD-L2; signal peptide is italicized; extracellular domain is underlined;
cytoplasmic domain is bolded)
SEQ ID NO: 175
MIFLLLMLSLELQLHQIAALFTVTVPKELYIIEHGSNVTLECNFDTGSHVNLGAITASLQKVENDTSPHRERAT
LLEEQLPLGKASFHIPQVQVRDEGQYQCIIIYGVAWDYKYLTLKVKASYRKINTHILKVPETDEVELTCQAT
GYPLAEVSWPNVSVPANTSHSRTPEGLYQVTSVLRLKPPPGRNFSCVFWNTHVRELTLASIDLQSQMEPRT
HPTWLLHIFIPFCIIAFIFIATVIALRKQLCQKLYSSKDTTKRPVTTTKREVNSAI
(wildtype human PD-L2 extracellular domain)
SEQ ID NO: 176
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
(wildtype human PD-L2 extracellular domain-hinge-IgG1 Fc mutant; PD-L2 is underlined;
hinge is bolded)
SEQ ID NO: 177
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTDKTHTCPPCPAPEL
LGGPSVFLFPPKPKDTLMISRTPEVTCVVVDVSHEDPEVKFNWYVDGVEVHNAKTKPREEQYASTYRVVS
VLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSP
GK
(wildtype human PD-L2 extracellular domain-hinge-IgG1 Fc mutant2; PD-L2 is
underlined; hinge is bolded)
SEQ ID NO: 178
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN

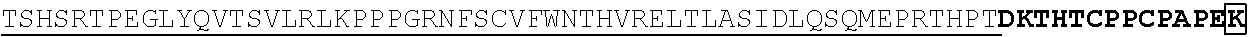

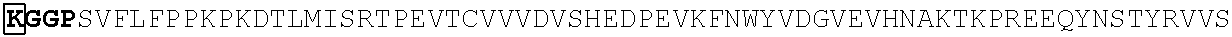

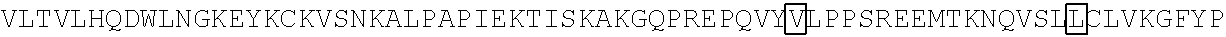

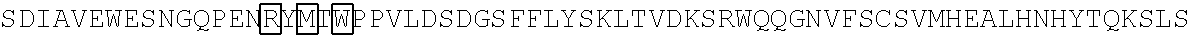

SPGK
(wildtype human PD-L2 extracellular domain-hinge-IgG1 Fc mutant2; PD-L2 is
underlined; linker is bolded and underlined; hinge is bolded)
SEQ ID NO: 179
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSDKTHTCPPC

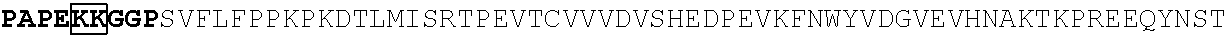

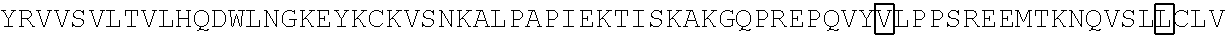

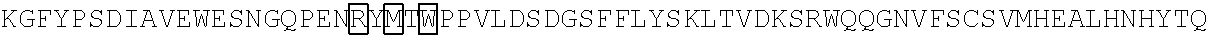

KSLSLSPGK
(wildtype human PD-L2 extracellular domain-linker-IL-2 mutant R38D/K43E/E61R-hinge-
IgG1 Fc mutant1; PD-L2 is underlined; linker is bolded and underlined; hinge is bolded; IL-2
mutant is italicized)
SEQ ID NO: 180
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSAPTSSSTKKT

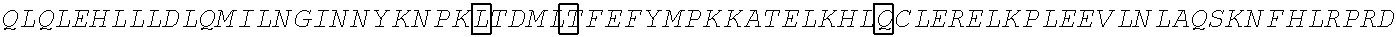

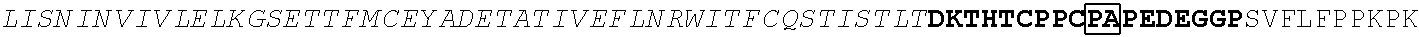

DTLMISRTPEVTCVVVDVSHEDPEVKFNWYVDGVEVHNAKTKPREEQYNSTYRVVSVLTVLHQDWLNGK

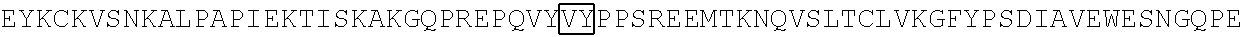

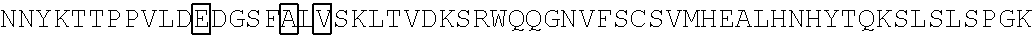

(wildtype human PD-L2 extracellular domain-linker-hinge-IgG1 Fc mutant2; PD-L2 is
underlined; linker is bolded and underlined; hinge is bolded)
SEQ ID NO: 181
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGGGGGDKTHTC

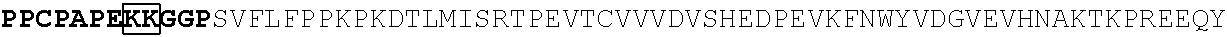

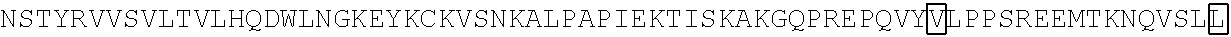

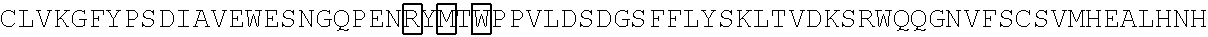

YTQKSLSLSPGK
(wildtype human PD-L2 extracellular domain-linker-IFN-α2b mutant L30A-hinge-IgG1 Fc
mutant1; PD-L2 is underlined; linker is bolded and underlined; hinge is bolded; IFN-α2b mutant
is italicized)
SEQ ID NO: 182
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCOATGYPLAEVSWPNVSVPAN
TSHSRTPEGLYQVTSVLRLKPPPGRNFSCVFWNTHVRELTLASIDLQSQMEPRTHPTGSGGGGGCDLPQTH

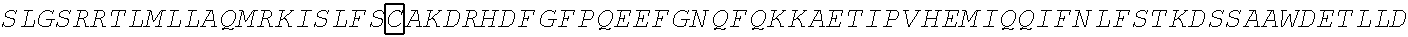

KFYTELYQQLNDLEACVIQGVGVTETPLMKEDSILAVRKYFORITLYLKEKKYSPCAWEVVRAEIMRSFSLSTNLQE

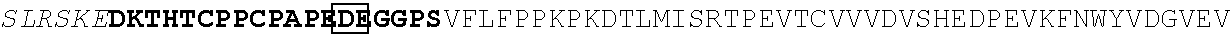

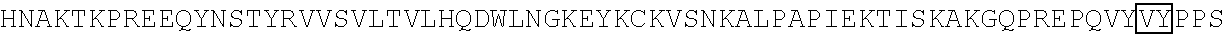

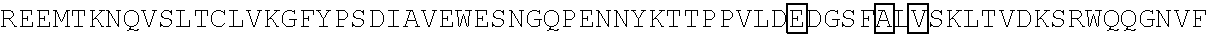

SCSVMHEALHNHYTQKSLSLSPGK
(wildtype human PD-L2 extracellular domain-linker-single-chain IFN-γ mutant A23V
homodimer-hinge-IgG1 Fc mutant1; PD-L2 is underlined; linker is bolded and underlined; hinge is bolded;
IFN-γ mutant monomer is italicized)
SEQ ID NO: 183
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGGGGGQDPYVKE

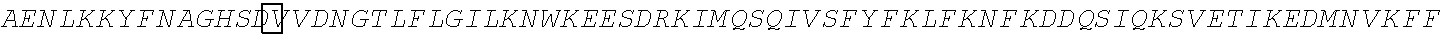

NSNKKKRDDFEKLTNYSVTDLNVQRKAIHELIQVMAELSPAAKTGKRKRSQMLFRGFEGGGSGGGGSGGGGS

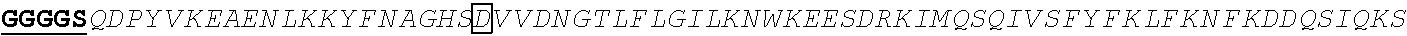

VETIKEDMNVKFFNSNKKKRDDFEKLTNYSVTDLNVORKAIHELIQVMAELSPAAKTGKRKRSQMLFRGDKTHT

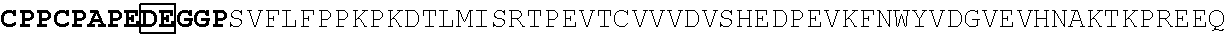

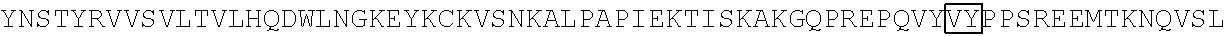

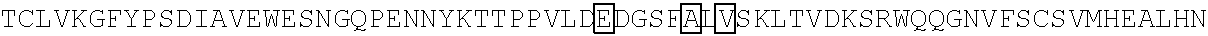

HYTQKSLSLSPGK
(wildtype human PD-L2 extracellular domain-linker-single-chain IL-10 mutant R27A
homodimer-hinge-IgG1 Fc mutant1; PD-L2 is underlined; linker is bolded and underlined; hinge is bolded;
IL-10 mutant monomer is italicized)
SEQ ID NO: 184
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGGGGGSPGQGTQ

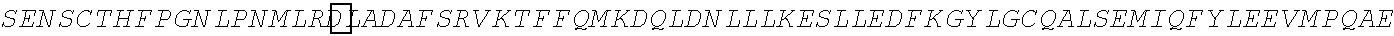

NQDPDIKAHVNSLGENLKTLRLRLRRCHRFLPCENKSKAVEQVKNAFNKLOEKGIYKAMSEFDIFINYIEAYMTM

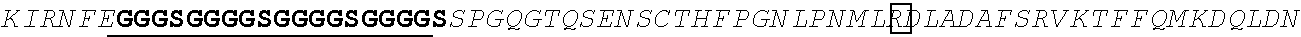

LLLKESLLEDFKGYLGCQALSEMIQFYLEEVMPQAENQDPDIKAHVNSLGENLKTLRLRLRRCHRFLPCENKSKA

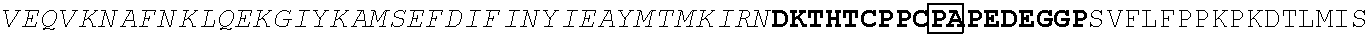

RTPEVTCVVVDVSHEDPEVKFNWYVDGVEVHNAKTKPREEQYNSTYRVVSVLTVLHQDWLNGKEYKCK

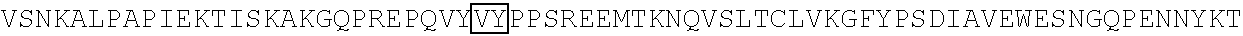

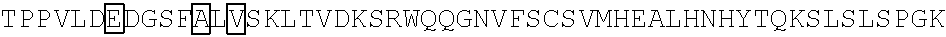

(wildtype human PD-L2 extracellular domain-hinge-IgG1 Fc mutant2; PD-L2 is
underlined; hinge is bolded; linker is bolded and underlined)
SEQ ID NO: 185
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGDKTHTCPPCPA

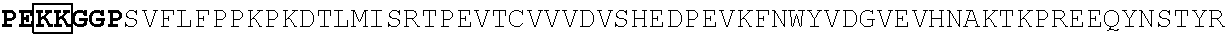

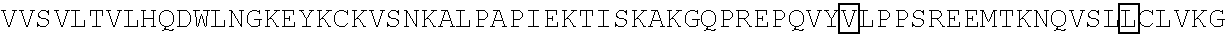

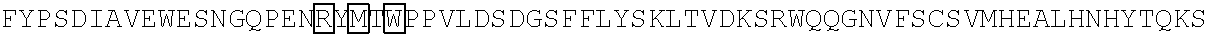

LSLSPGK
(wildtype human PD-L2 extracellular domain-linker-single-chain IL-12 mutant
heterodimer IL-12B (p40 E59A/F60A)-linker-IL-12A (wt p35)-hinge-IgG1 Fc mutant1; PD-L2 is underlined;
linker is bolded and underlined; hinge is bolded; IL-12 subunits are italicized)
SEQ ID NO: 186
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGIWELKKDVYVVEL

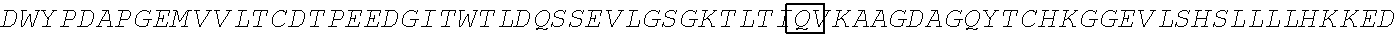

GIWSTDILKDOKEPKNKTFLRCEAKNYSGRFTCWWLTTISTDLTFSVKSSRGSSDPQGVTCGAATLSAERVRGDNK
EYEYSVECQEDSACPAAEESLPIEVMVDAVHKLKYENYTSSFFIRDIIKPDPPKNLQLKPLKNSRQVEVSWEYPDTW
STPHSYFSLTFCVQVQGKSKREKKDRVFTDKTSATVICRKNASISVRAQDRYYSSSWSEWASVPCSGGGGSGGGG
SGGGGSGGGGSGRNLPVATPDPGMFPCLHHSQNLLRAVSNMLQKARQTLEFYPCTSEEIDHEDITKDKTSTVE
ACLPLELTKNESCLNSRETSFITNGSCLASRKTSFMMALCLSSIYEDLKMYQVEFKTMNAKLLMDPKRQIFLDQNM

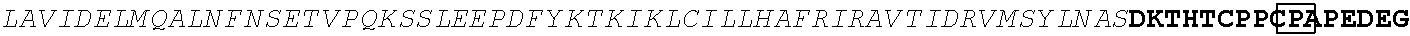

GPSVFLFPPKPKDTLMISRTPEVTCVVVDVSHEDPEVKFNWYVDGVEVHNAKTKPREEQYNSTYRVVSVL

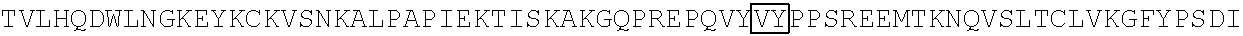

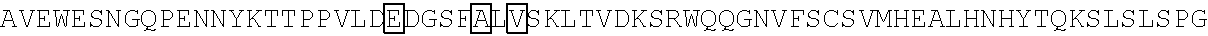

K
(wildtype human PD-L2 extracellular domain-linker-single-chain IL-23 mutant
heterodimer IL-12B (p40 E59A/F60A)-linker-IL-23A (wt p19)-hinge-IgG1 Fc mutant1; PD-L2 is underlined;
linker is bolded and underlined; hinge is bolded; IL-23 subunits are italicized)
SEQ ID NO: 187
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGIWELKKDVYVVEL

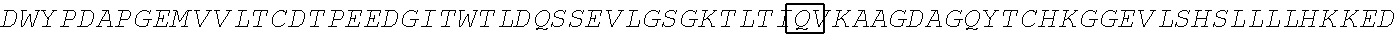

GIWSTDILKDQKEPKNKTFLRCEAKNYSGRFTCWWLTTISTDLTFSVKSSRGSSDPQGVTCGAATLSAERVRGDNK
EYEYSVECQEDSACPAAEESLPIEVMVDAVHKLKYENYTSSFFIRDIIKPDPPKNLOLKPLKNSRQVEVSWEYPDTW
STPHSYFSLTFCVQVQGKSKREKKDRVFTDKTSATVICRKNASISVRAQDRYYSSSWSEWASVPCSGGGGSGGGG
SGGGGSGGGGSGRAVPGGSSPAWTQCQQLSQKLCTLAWSAHPLVGHMDLREEGDEETTNDVPHIQCGDGCD
PQGLRDNSQFCLQRIHQGLIFYEKLLGSDIFTGEPSLLPDSPVGQLHASLLGLSQLLQPEGHHWETQQIPSLSPSQ

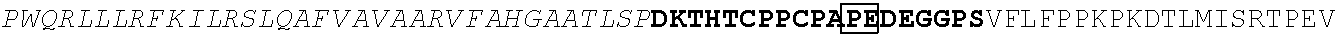

TCVVVDVSHEDPEVKFNWYVDGVEVHNAKTKPREEQYNSTYRVVSVLTVLHQDWLNGKEYKCKVSNKA

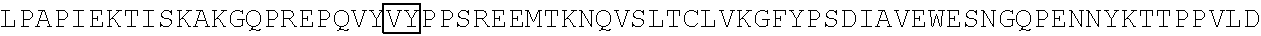

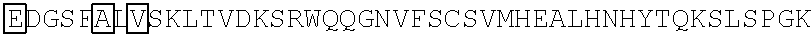

(linker; n is an integer of at least 1)
SEQ ID NO: 194
(G)n
(linker; n is an integer of at least 1)
SEQ ID NO: 195
(GS)n
(linker; n is an integer of at least 1)
SEQ ID NO: 196
(GGS)n
(linker; n is an integer of at least 1)
SEQ ID NO: 197
(GGGS)n
(linker; n is an integer of at least 1)
SEQ ID NO: 198
(GGS)n(GGGS)n
(linker; n is an integer of at least 1)
SEQ ID NO: 199
(GSGGS)n
linker; n is an integer of at least 1)
SEQ ID NO: 200
(GGSGS)ₙ
(linker; n is an integer of at least 1)
SEQ ID NO: 201
(GGGGS)n
(linker)
SEQ ID NO: 202
GG
(linker)
SEQ ID NO: 203
GSG
(linker)
SEQ ID NO: 204
GGSG
(linker)
SEQ ID NO: 205
GGSGG
(linker)
SEQ ID NO: 206
GSGGGGG
(linker)
SEQ ID NO: 207
GSGSG
(linker)
SEQ ID NO: 208
GSGGG
(linker)
SEQ ID NO: 209
GGGSG
(linker)
SEQ ID NO: 210
GSSSG
(linker)
SEQ ID NO: 211
GGSGGS
(linker)
SEQ ID NO: 212
SGGGGS
(linker)
SEQ ID NO: 213
GGGGS
(linker; n is an integer of at least 1)
SEQ ID NO: 214
(GA)n
(linker)
SEQ ID NO: 215
GRAGGGGAGGGG
(linker)
SEQ ID NO: 216
GRAGGG
(linker)
SEQ ID NO: 217
GSGGGSGGGGSGGGGS
(linker)
SEQ ID NO: 218
GGGSGGGGSGGGGS
(linker)
SEQ ID NO: 219
GGGSGGSGGS
(linker)
SEQ ID NO: 220
GGSGGSGGSGGSGGG
(linker)
SEQ ID NO: 221
GGSGGSGGGGSGGGGS
(linker)
SEQ ID NO: 222
GGSGGSGGSGGSGGSGGS
(linker)
SEQ ID NO: 223
GGGGGGSGGGGSGGGGSA
(linker)
SEQ ID NO: 224
GSGGGSGGGGSGGGGSGGGGS
(linker)
SEQ ID NO: 225
KTGGGSGGGS
(linker)
SEQ ID NO: 226
GGPGGGGSGGGSGGGGS
(linker)
SEQ ID NO: 227
GGGSGGGGSGGGGSGGGGS
(linker)
SEQ ID NO: 228
GGGGSGGGGSGGGGSGGGGSG
(linker)
SEQ ID NO: 229
GGGGSGGGGSGGGGS
(linker)
SEQ ID NO: 230
ASTK GP
(linker)
SEQ ID NO: 231
DKP
(linker)
SEQ ID NO: 232
DKPGS
(linker)
SEQ ID NO: 233
PGS
(linker)
SEQ ID NO: 234
GS
(linker)
SEQ ID NO: 235
DKPGSG
(linker)
SEQ ID NO: 236
PGSG
(linker)
SEQ ID NO: 237
DKPGSGS
(linker)
SEQ ID NO: 238
PGSGS
(linker)
SEQ ID NO: 239
GSGS
(linker)
SEQ ID NO: 240
DKPGSGGGGG
(linker)
SEQ ID NO: 241
PGSGGGGG
(linker)
SEQ ID NO: 242
P
(wildtype human PD-L1; signal peptide is italicized; extracellular domain is underlined;
cytoplasmic domain is bolded)
SEQ ID NO: 249
MRIFAVFIFMTYWHLLNAFTVTVPKDLYVVEYGSNMTIECKFPVEKQLDLAALIVYWEMEDKNIIQFVHGEE
DLKVQHSSYRQRARLLKDOLSLGNAALQITDVKLQDAGVYRCMISYGGADYKRITVKVNAPYNKINQRIL
VVDPVTSEHELTCQAEGYPKAEVIWTSSDHQVLSGKTTTTNSKREEKLFNVTSTLRINTTTNEIFYCTFRRL
DPEENHTAELVIPELPLAHPPNERTHLVILGAILLCLGVALTFIFRLRKGRMMDVKKCGIQDTNSKKQSDT
HLEET
(wildtype human PD-L1 extracellular domain)
SEQ ID NO: 250
FTVTVPKDLYVVEYGSNMTIECKFPVEKQLDLAALIVYWEMEDKNIIQFVHGEEDLKVQHSSYRQRARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL
AHPPNER
SEQ ID NO: 251 (IL-2 mutant L18R/Q22E/R38D/K43E/E61R)

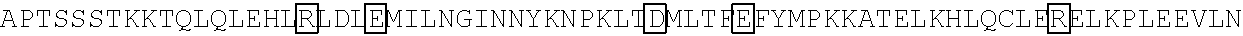

LAQSKNFHLRPRDLISNINVIVLELKGSETTFMCEYADETATIVEFLNRWITFCQSIISTLT
(IL-2 mutant R38D/K43E/E61R/Q126T)
SEQ ID NO: 252

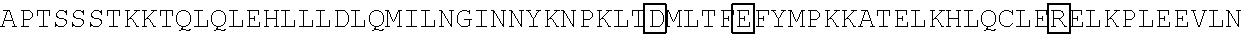

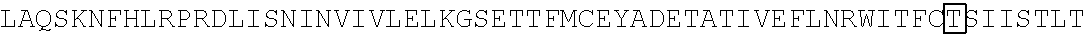

(IL-2 mutant L18R/Q22E/R38D/K43E/E61R/Q126T)
SEQ ID NO: 253

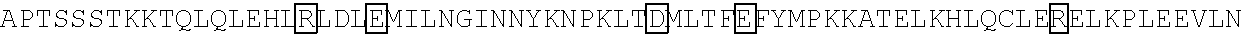

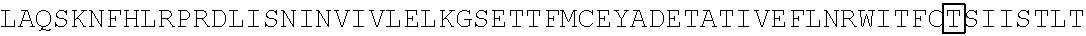

(IL-2 mutant L18R/Q22E/R38D/K43E/E61R/Q126T/S130R)
SEQ ID NO: 254

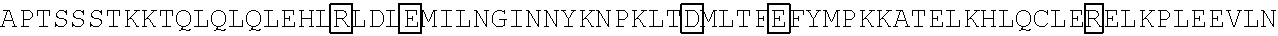

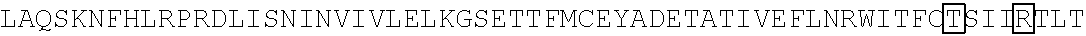

(human PD-L1 extracellular domain mutant1 E58M/R113T/M115L/S117A/G119K)
SEQ ID NO: 255

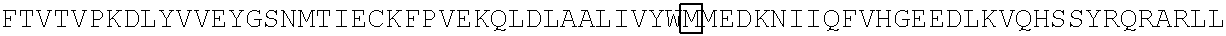

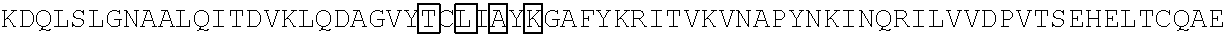

GYPKAEVIWTSSDHQVLSGKTTTTNSKREEKLFNVTSTLRINTTTNEIFYCTFRRLDPEENHTAELVIPELPL
AHPPNER
(human PD-L1 extracellular domain mutant2 I54Q/E58M/R113T/M115L/S117A/G119K)
SEQ ID NO: 256

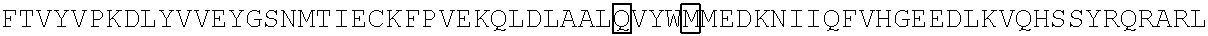

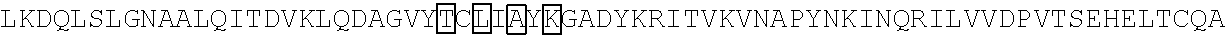

EGYPKAEVIWTSSDHQVLSGKTTTTNSKREEKLFNVTSTLRINTTTNEIFYCTFRRLDPEENHTAEL VIPELPL
AHPPNER
(human PD-L1 extracellular domain mutant3 I54Q/R113T/M115L/S117A/G119K)
SEQ ID NO: 257

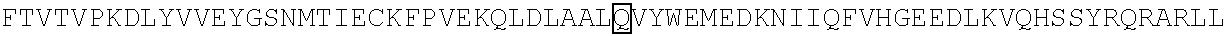

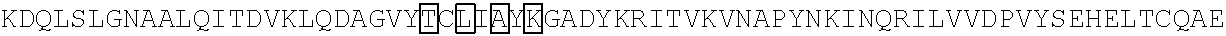

GYPKAEVIWTSSDHQVLSGKTTTTNSKREEKLFNVTSTLRINTTTNEIFYCTFRRLDPEENHTAEL VIPELPL
AHPPNER
(human PD-L1 extracellular domain mutant4 I54Q/E58M/M115L/S117A/G119K)
SEQ ID NO: 258

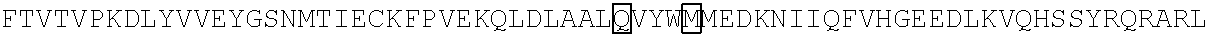

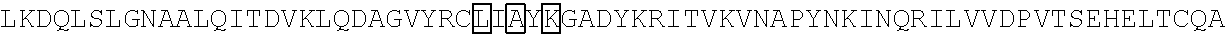

EGYPKAEVIWTSSDHQVLSGKTTTTNSKREEKLFNVTSTLRINTTTNEIFYCTFRRLDPEENHTAELVIPELPL
AHPPNER
(human PD-L1 extracellular domain mutant5 154Q/E58M/R113T/S117A/G119K)
SEQ ID NO: 259

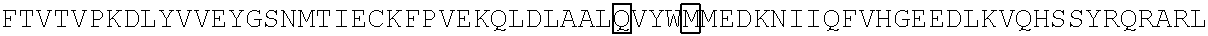

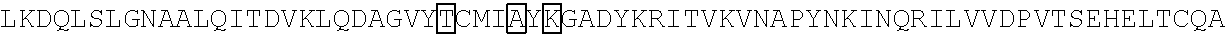

EGYPKAEVIWTSSDHQVLSGKTTTTNSKREEKLFNVTSTLRINTTTNEIFYCTFRRLDPEENHTAELVIPELPL
AHPPNER
(human PD-L1 extracellular domain mutant6 154Q/E58M/R113T/M115L/G119K)
SEQ ID NO: 260

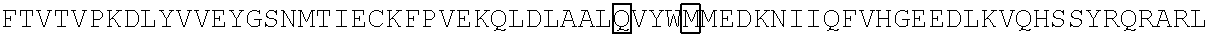

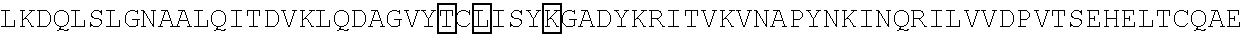

GYPKAEVIWTSSDHQVLSGKTTTTNSKREEKLFNVTSTLRINTTTNEIFYCTFRRLDPEENHTAELVIPELPL
AHPPNER
(human PD-L1 extracellular domain mutant7 154Q/E58M/R113T/M115L/S117A)
SEQ ID NO: 261

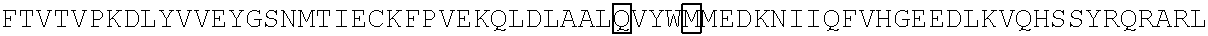

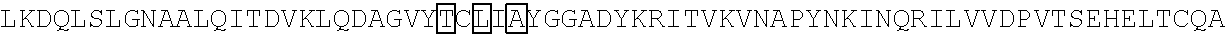

EGYPKAEVIWTSSDHQVLSGKTTTTNSKREEKLFNVTSTLRINTTTNEIFYCTFRRLDPEENHTAELVIPELPL
AHPPNER
(human PD-L2 extracellular domain mutant1 T56V)
SEQ ID NO: 262

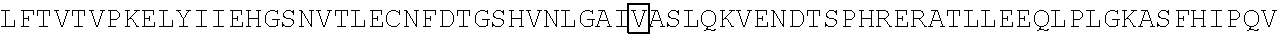

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
(human PD-L2 extracellular domain mutant2 S58V)
SEQ ID NO: 263

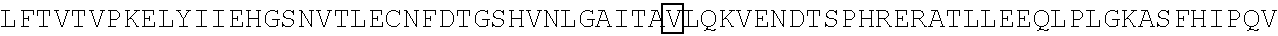

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
(human PD-L2 extracellular domain mutant3 Q60L)
SEQ ID NO: 264

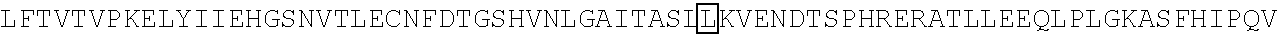

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
(human PD-L2 extracellular domain mutant4 T56V/S58V/Q60L)
SEQ ID NO: 265

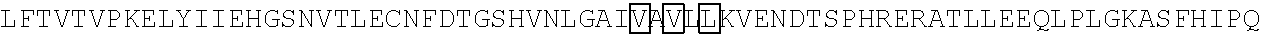

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPT
(Wild-type human CD155)
SEQ ID NO: 266
MARAMAAAWPLLLVALLVLSWPPPGTGDVVVQAPTQVPGFLGDSVTLPCYLQVPNMEVTHVSQLTWAR
HGESGSMAVFHQTQGPSYSESKRLEFVAARLGAELRNASLRMFGLRVEDEGNYTCLFVTFPQGSRSVDIWL
RVLAKPQNTAEVQKVQLTGEPVPMARCVSTGGRPPAQITWHSDLGGMPNTSQVPGFLSGTVTVTSLWILV
PSSQVDGKNVTCKVEHESFEKPQLLTVNLTVYYPPEVSISGYDNNWYLGQNEATLTCDARSNPEPTGYNW
STTMGPLPPFAVAQGAQLLIRPVDKPINTTLICNVTNALGARQAELTVQVKEGPPSEHSGISRNAIIFLVLGIL
VFLILLGIGIYFYWSKCSREVLWHCHLCPSSTEHASASANGHVSYSAVSRENSSSQDPQTEGTR
(Wild-type human CD155 extracellular domain)
SEQ ID NO: 267
WPPPGTGDVVVQAPTQVPGFLGDSVTLPCYLQVPNMEVTHVSQLTWARHGESGSMAVFHQ
TQGPSYSESKRLEFVAARLGAELRNASLRMFGLRVEDEGNYTCLFVTFPQGSRSVDIWLR
VLAKPQNTAEVQKVQLTGEPVPMARCVSTGGRPPAQITWHSDLGGMPNTSQVPGFLSGTV
TVTSLWILVPSSQVDGKNVTCKVEHESFEKPQLLTVNLTVYYPPEVSISGYDNNWYLGQN
EATLTCDARSNPEPTGYNWSTTMGPLPPFAVAQGAQLLIRPVDKPINTTLICNVTNALGA
RQAELTVQVKEGPPSEHSGISRN
(anti-PD-1 Ab VH(D100N)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 268
QVQLVESGGGVVQPGRSLRLDCKASGITFSNSGMHWVRQAPGKGLEWVAVIWYDGSKRYYADSVKGRF

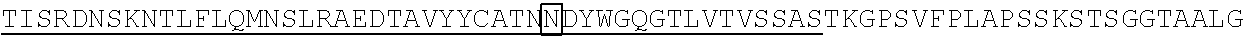

CLVKDYFPEPVTVSWNSGALTSGVHTFPAVLQSSGLYSLSSVVTVPSSSLGTQTYICNVNHKPSNTKVDKK

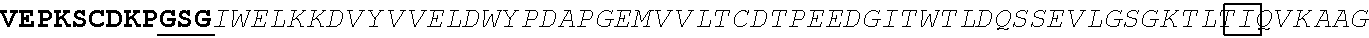

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKROIFLDONMLAVIDELMQALNFNSETVPOKSSLEEPDFYKTKIKLCILLHAFRIRAVTID

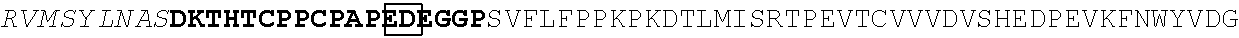

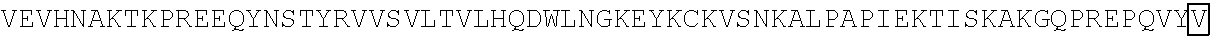

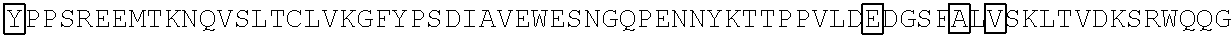

NVFSCSVMHEALHNHYTQKSLSLSPGK
(anti-PD-1 Ab VH(D100G)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 269
QVQLVESGGGVVQPGRSLRLDCKASGITFSNSGMHWVRQAPGKGLEWVAVIWYDGSKRYYADSVKGRF

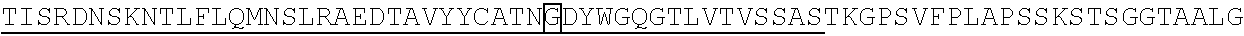

CLVKDYFPEPVTVSWNSGALTSGVHTFPAVLQSSGLYSLSSVVTVPSSSLGTQTYICNVNHKPSNTKVDKK

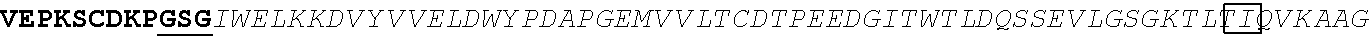

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNFNSETVPQKSSLEEPDFYKTKIKLCILLHAFRIRAVTID

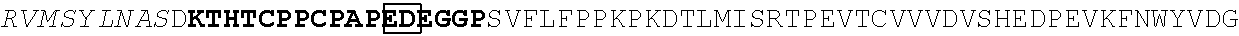

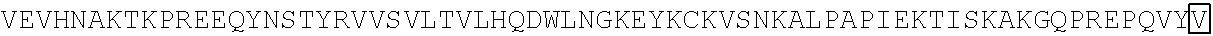

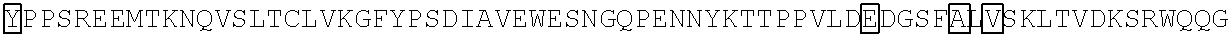

NVFSCSVMHEALHNHYTQKSLSLSPGK
(anti-PD-1 Ab VH(D100R)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 270
QVQLVESGGGVVQPGRSLRLDCKASGITFSNSGMHWVRQAPGKGLEWVAVIWYDGSKRYYADSVKGRF

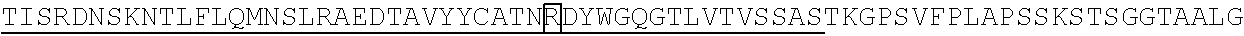

CLVKDYFPEPVTVSWNSGALTSGVHTFPAVLQSSGLYSLSSVVTVPSSSLGTQTYICNVNHKPSNTKVDKK

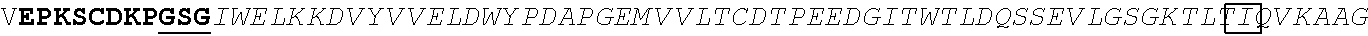

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSROVEVSWEYPDTWSTPHSYFSLTFCVOVOGKSKREKKDRVFTDKTSATVICRKNASISVRAQD
RYYSSSWSEWASVPCSGGGGSGGGGSGGGGSGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNFNSETVPQKSSLEEPDFYKTKIKLCILLHAFRIRAVTID

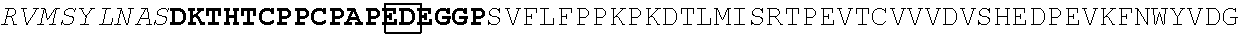

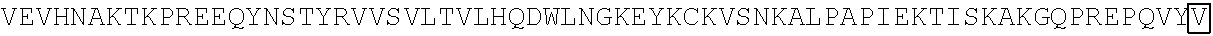

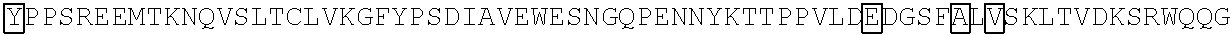

NVFSCSVMHEALHNHYTQKSLSLSPGK
(anti-PD-1 Ab VH(N99G)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 271
QVQLVESGGGVVQPGRSLRLDCKASGITFSNSGMHWVRQAPGKGLEWVAVIWYDGSKRYYADSVKGRF

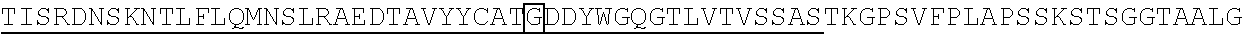

CLVKDYFPEPVTVSWNSGALTSGVHTFPAVLQSSGLYSLSSVVTVPSSSLGTQTYICNVNHKPSNTKVDKK

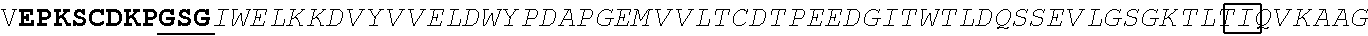

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVOVOGKSKREKKDRVFTDKTSATVICRKNASISVRAQD
RYYSSSWSEWASVPCSGGGGSGGGGSGGGGSGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNFNSETVPQKSSLEEPDFYKTKIKLCILLHAFRIRAVTID

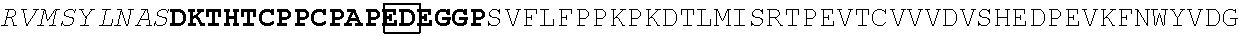

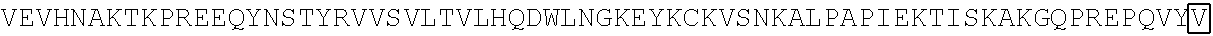

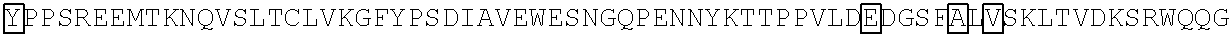

NVFSCSVMHEALHNHYTQKSLSLSPGK
(anti-PD-1 Ab VH(N99A)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 272
QVQLVESGGGVVQPGRSLRLDCKASGITFSNSGMHWVRQAPGKGLEWVAVIWYDGSKRYYADSVKGRF

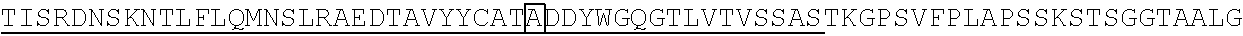

CLVKDYFPEPVTVSWNSGALTSGVHTFPAVLQSSGLYSLSSVVTVPSSSLGTQTYICNVNHKPSNTKVDKK

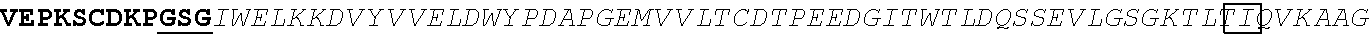

DAGQYTCHKGGEVLSHSLLLLHKKEDGIWSTDILKDOKEPKNKTFLRCEAKNYSGRFTCWWLTTISTDLTFSVKSS
RGSSDPQGVTCGAATLSAERVRGDNKEYEYSVECOEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDONMLAVIDELMQALNFNSETVPQKSSLEEPDFYKTKIKLCILLHAFRIRAVTID

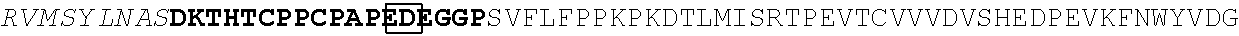

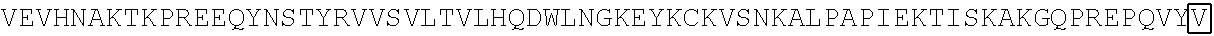

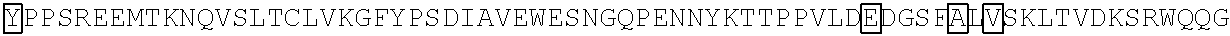

NVFSCSVMHEALHNHYTQKSLSLSPGK
(anti-PD-1 Ab VH(N99M)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 273
QVQLVESGGGVVQPGRSLRLDCKASGITFSNSGMHWVRQAPGKGLEWVAVIWYDGSKRYYADSVKGRF

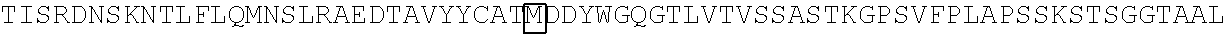

GCLVKDYFPEPVTVSWNSGALTSGVHTFPAVLQSSGLYSLSSVVTVPSSSLGTQTYICNVNHKPSNTKVDK

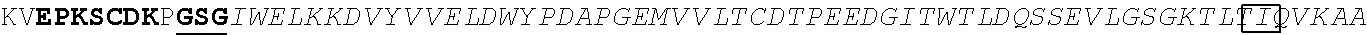

GDAGQYTCHKGGEVLSHSLLLLHKKEDGIWSTDILKDOKEPKNKTFLRCEAKNYSGRFTCWWLTTISTDLTFSVK
SSRGSSDPQGVTCGAATLSAERVRGDNKEYEYSVECOEDSACPAAEESLPIEVMVDAVHKLKYENYTSSFFIRDIIK
PDPPKNLOLKPLKNSROVEVSWEYPDTWSTPHSYFSLTFCVOVOGKSKREKKDRVFTDKTSATVICRKNASISVRA
QDRYYSSSWSEWASVPCSGGGGSGGGGSGGGGSGGGGSGRNLPVATPDPGMFPCLHHSQNLLRAVSNMLQK
ARQTLEFYPCTSEEIDHEDITKDKTSTVEACLPLELTKNESCLNSRETSFITNGSCLASRKTSFMMALCLSSIYEDLK
MYQVEFKTMNAKLLMDPKRQIFLDQNMLAVIDELMQALNFNSETVPQKSSLEEPDFYKTKIKLCILLHAFRIRAVT

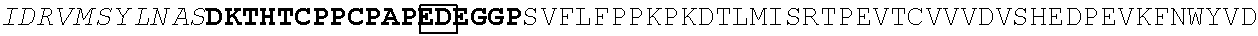

GVEVHNAKTKPREEQYNSTYRVVSVLTVLHQDWLNGKEYKCKVSNKALPAPIEKTISKAKGQPREPQVY

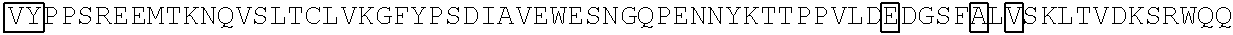

GNVFSCSVMHEALHNHYTQKSLSLSPGK
(human PD-L2 extracellular domain hinge portion-linker-single-chain IL-12 mutant
heterodimer IL-12B (p40 F60A)-linker IL-12A (wt p35)-C' hinge portion-IgG1 Fc mutant1; VH is
underlined; hinge is bolded; linker is bolded and underlined; IL-12 subunits are italicized)
SEQ ID NO: 274
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTGSGIWELKKDVYVVELDWYPDAPGEMVVLTCD

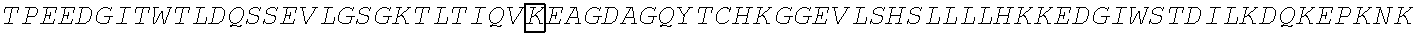

TFLRCEAKNYSGRFTCWWLTTISTDLTFSVKSSRGSSDPQGVTCGAATLSAERVRGDNKEYEYSVECQEDSACPAA
EESLPIEVMVDAVHKLKYENYTSSFFIRDIIKPDPPKNLOLKPLKNSRQVEVSWEYPDTWSTPHSYFSLTFCVQVQG
KSKREKKDRVFTDKTSATVICRKNASISVRAQDRYYSSSWSEWASVPCSGGGGSGGGGSGGGGSGGGGSGRNL
PVATPDPGMFPCLHHSONLLRAVSNMLQKARQTLEFYPCTSEEIDHEDITKDKTSTVEACLPLELTKNESCLNSRE
TSFITNGSCLASRKTSFMMALCLSSIYEDLKMYQVEFKTMNAKLLMDPKRQIFLDQNMLAVIDELMQALNFNSET

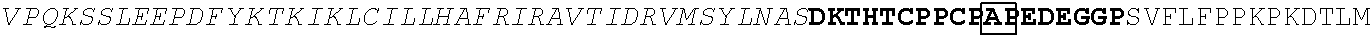

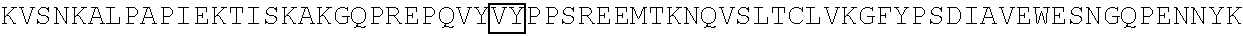

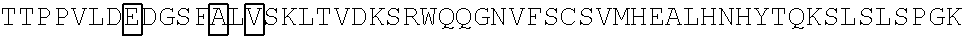

TTPPVLDEDGSFALVSKLTVDKSRWQQGNVFSCSVMHEALHNHYTQKSLSLSPGK
(single-chain IL-12 mutant heterodimer IL-12B (p40 F60A)-linker-IL-12A (wt p35); linker
is bolded; IL-12 subunits are italicized)
SEQ ID NO: 275

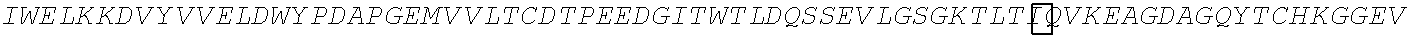

LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKTSTVEACLPLELTKNESCLNSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKLCILLHAFRIRAVTIDRVMSYLNAS
(human PD-L2 extracellular domain-linker-hinge portion-IgG1 Fc mutant1-linker-single-
chain IL-12 mutant heterodimer IL-12B (p40 F60A)-linker IL-12A (wt p35); VH is underlined; hinge is
bolded; linker is bolded and underlined; IL-12 subunits are italicized)
SEQ ID NO: 276
LFTVTVPKELYIIEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN

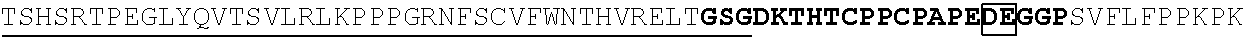

DTLMISRTPEVTCVVVDVSHEDPEVKFNWYVDGVEVHNAKTKPREEQYNSTYRVVSVLTVLHQDWLNGK

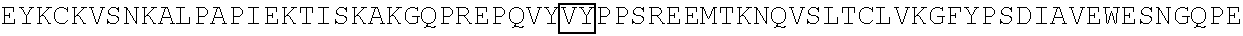

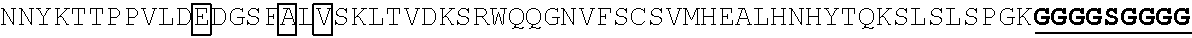

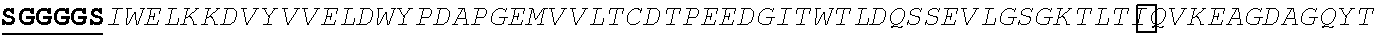

CHKGGEVLSHSLLLLHKKEDGIWSTDILKDOKEPKNKTFLRCEAKNYSGRFTCWWLTTISTDLTFSVKSSRGSSDP
QGVTCGAATLSAERVRGDNKEYEYSVECQEDSACPAAEESLPIEVMVDAVHKLKYENYTSSFFIRDIIKPDPPKNL
QLKPLKNSRQVEVSWEYPDTWSTPHSYFSLTFCVQVQGKSKREKKDRVFTDKTSATVICRKNASISVRAODRYYSSS
WSEWASVPCSGGGGSGGGGSGGGGSGGGGSGRNLPVATPDPGMFPCLHHSQNLLRAVSNMLQKARQTLEF
YPCTSEEIDHEDITKDKTSTVEACLPLELTKNESCLNSRETSFITNGSCLASRKTSFMMALCLSSIYEDLKMYQVEFK
TMNAKLLMDPKRQIFLDQNMLAVIDELMQALNFNSETVPQKSSLEEPDFYKTKIKLCILLHAFRIRAVTIDRVMSY
LNAS
(human PD-L1 extracellular domain mutant2 I54Q/E58M/R113T/M115L/S117A/G119K
extracellular domain-linker-single-chain IL-12 mutant heterodimer IL-12B (p40 E59A/F60A)-linker IL-12A
(wt p35)-hinge-IgG1 Fc mutant1; PD-L1 is underlined; linker is bolded and underlined; hinge is bolded; IL-
12 subunits are italicized)
SEQ ID NO: 277

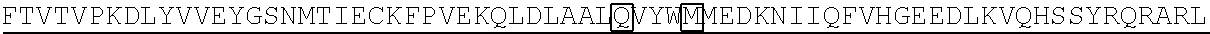

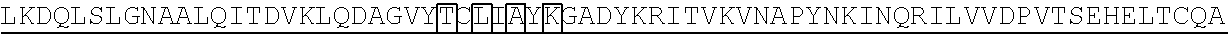

EGYPKAEVIWTSSDHQVLSGKTTTTNSKREEKLFNVTSTLRINTTTNEIFYCTFRRLDPEENHTAELVIPELPL
AHPPNERGGGGSGGGIWELKKDVYVVELDWYPDAPGEMVVLTCDTPEEDGITWTLDQSSEVLGSGKTLTIQVK

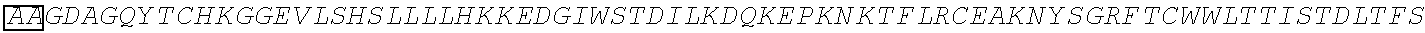

VKSSRGSSDPQGVTCGAATLSAERVRGDNKEYEYSVECQEDSACPAAEESLPIEVMVDAVHKLKYENYTSSFFIRDI
IKPDPPKNLOLKPLKNSRQVEVSWEYPDTWSTPHSYFSLTFCVQVQGKSKREKKDRVFTDKTSATVICRKNASISV
RAQDRYYSSSWSEWASVPCSGGGGSGGGSGGGGSRNLPVATPDPGMFPCLHHSQNLLRAVSNMLQKARQTLE
FYPCTSEEIDHEDITKDKTSTVEACLPLELTKNESCLNSRETSFITNGSCLASRKTSFMMALCLSSIYEDLKMYQVEF
KTMNAKLLMDPKRQIFLDONMLAVIDELMQALNFNSETVPQKSSLEEPDFYKTKIKLCILLHAFRIRAVTIDRVMS

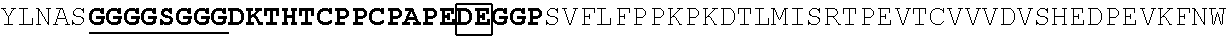

YVDGVEVHNAKTKPREEQYNSTYRVVSVLTVLHQDWLNGKEYKCKVSNKALPAPIEKTISKAKGQPREPQ

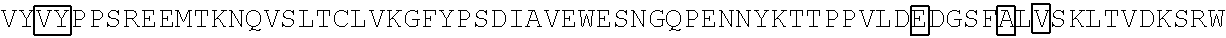

QQGNVFSCSVMHEALHNHYTQKSLSLSPGK
(human PD-L1 extracellular domain mutant7 I54Q/E58M/R113T/M115L/S117A
extracellular domain-linker-single-chain IL-12 mutant heterodimer IL-12B (p40 E59A/F60A)-linker IL-12A
(wt p35)-hinge-IgG1 Fc mutant1; PD-L1 is underlined; linker is bolded and underlined; hinge is bolded; IL-
12 subunits are italicized)
SEQ ID NO: 278

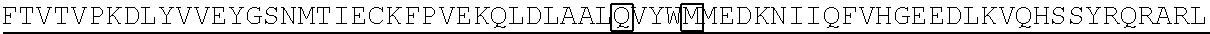

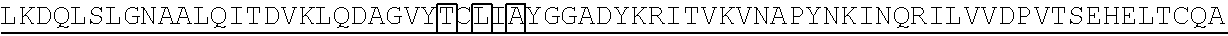

EGYPKAEVIWTSSDHQVLSGKTTTTNSKREEKLFNVTSTLRINTTTNEIFYCTFRRLDPEENHTAELVIPELPL
AHPPNERGGGGSGGGIWELKKDVYVVELDWYPDAPGEMVVLTCDTPEEDGITWTLDQSSEVLGSGKTLTIQVK

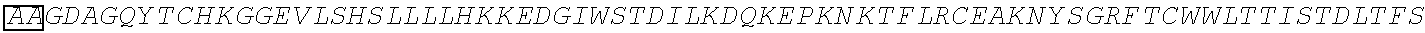

VKSSRGSSDPQGVTCGAATLSAERVRGDNKEYEYSVECQEDSACPAAEESLPIEVMVDAVHKLKYENYTSSFFIRDI
IKPDPPKNLOLKPLKNSROVEVSWEYPDTWSTPHSYFSLTFCVOVOGKSKREKKDRVFTDKTSATVICRKNASISV
RAQDRYYSSSWSEWASVPCSGGGGSGGGSGGGGSRNLPVATPDPGMFPCLHHSQNLLRAVSNMLQKARQTLE
FYPCTSEEIDHEDITKDKTSTVEACLPLELTKNESCLNSRETSFITNGSCLASRKTSFMMALCLSSIYEDLKMYQVEF
KTMNAKLLMDPKRQIFLDONMLAVIDELMQALNFNSETVPQKSSLEEPDFYKTKIKLCILLHAFRIRAVTIDRVMS

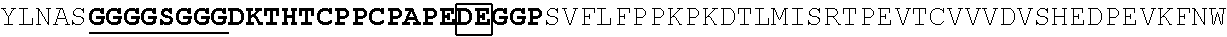

YVDGVEVHNAKTKPREEQYNSTYRVVSVLTVLHQDWLNGKEYKCKVSNKALPAPIEKTISKAKGQPREPQ

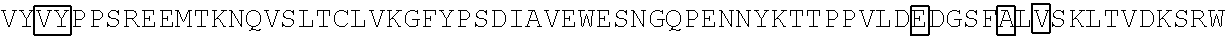

QQGNVFSCSVMHEALHNHYTQKSLSLSPGK
(human PD-L1 extracellular domain mutant2 154Q/E58M/R113T/M115L/S117A/G119K
extracellular domain-linker-hinge-IgG1 Fc mutant1-linker-single-chain IL-12 mutant heterodimer IL-12B
(p40 E59A/F60A)-linker IL-12A (wt p35); PD-L1 is underlined; linker is bolded and underlined; hinge is
bolded; IL-12 subunits are italicized)
SEQ ID NO: 279

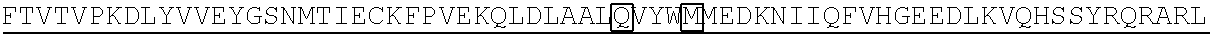

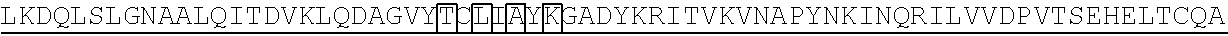

EGYPKAEVIWTSSDHQVLSGKTTTTNSKREEKLFNVTSTLRINTTTNEIFYCTFRRLDPEENHTAELVIPELPL

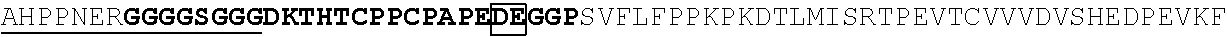

NWYVDGVEVHNAKTKPREEQYNSTYRVVSVLTVLHQDWLNGKEYKCKVSNKALPAPIEKTISKAKGQPR

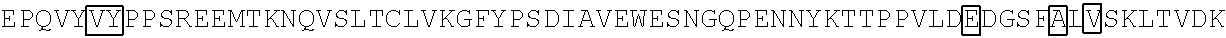

SRWQQGNVFSCSVMHEALHNHYTQKSLSLSPGKGGGGSGGGIWELKKDVYVVELDWYPDAPGEMVVLTCD

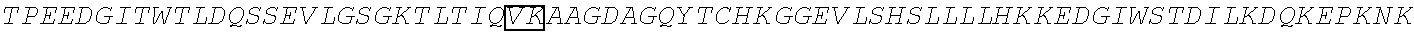

TFLRCEAKNYSGRFTCWWLTTISTDLTFSVKSSRGSSDPQGVTCGAATLSAERVRGDNKEYEYSVECQEDSACPAA
EESLPIEVMVDAVHKLKYENYTSSFFIRDIIKPDPPKNLOLKPLKNSROVEVSWEYPDTWSTPHSYFSLTFCVQVQG
KSKREKKDRVFTDKTSATVICRKNASISVRAQDRYYSSSWSEWASVPCSGGGGSGGGSGGGGSRNLPVATPDPG
MFPCLHHSONLLRAVSNMLQKARQTLEFYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNMLAVIDELMQALNFNSETVPQKSSLEE
PDFYKTKIKLCILLHAFRIRAVTIDRVMSYLNAS
(human PD-L1 extracellular domain mutant7 I54Q/E58M/R113T/M115L/S117A
extracellular domain-linker-hinge-IgG1 Fc mutant1-linker-single-chain IL-12 mutant heterodimer IL-12B
(p40 E59A/F60A)-linker IL-12A (wt p35); PD-L1 is underlined; linker is bolded and underlined; hinge is
bolded; IL-12 subunits are italicized)
SEQ ID NO: 280

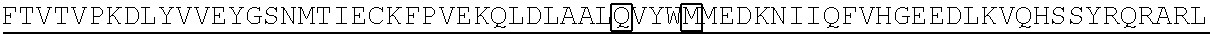

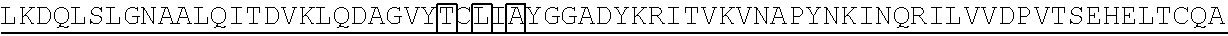

EGYPKAEVIWTSSDHQVLSGKTTTTNSKREEKLFNVTSTLRINTTTNEIFYCTFRRLDPEENHTAELVIPELPL

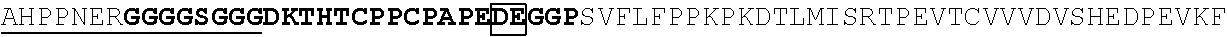

NWYVDGVEVHNAKTKPREEQYNSTYRVVSVLTVLHQDWLNGKEYKCKVSNKALPAPIEKTISKAKGQPR

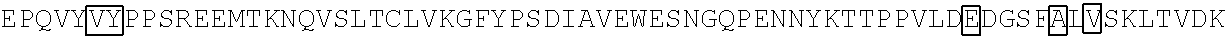

SRWQQGNVFSCSVMHEALHNHYTQKSLSLSPGKGGGGSGGGIWELKKDVYVVELDWYPDAPGEMVVLTCD

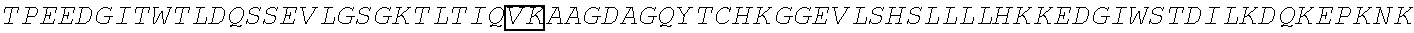

TFLRCEAKNYSGRFTCWWLTTISTDLTFSVKSSRGSSDPQGVTCGAATLSAERVRGDNKEYEYSVECQEDSACPAA
EESLPIEVMVDAVHKLKYENYTSSFFIRDIIKPDPPKNLQLKPLKNSRQVEVSWEYPDTWSTPHSYFSLTFCVQVQG
KSKREKKDRVFTDKTSATVICRKNASISVRAQDRYYSSSWSEWASVPCSGGGGSGGGSGGGGSRNLPVATPDPG
MFPCLHHSONLLRAVSNMLQKARQTLEFYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNMLAVIDELMQALNFNSETVPQKSSLEE
PDFYKTKIKLCILLHAFRIRAVTIDRVMSYLNAS
(human PD-L1 extracellular domain mutant2 154Q/E58M/R113T/M115L/S117A/G119K
extracellular domain-linker-hinge-IgG1 Fc mutant1; PD-L1 is underlined; linker is bolded and underlined;
hinge is bolded;
SEQ ID NO: 281

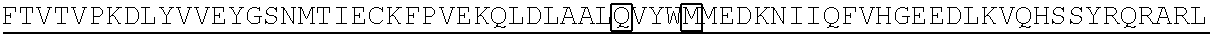

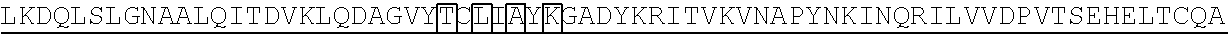

EGYPKAEVIWTSSDHQVLSGKTTTTNSKREEKLFNVTSTLRINTTTNEIFYCTFRRLDPEENHTAELVIPELPL

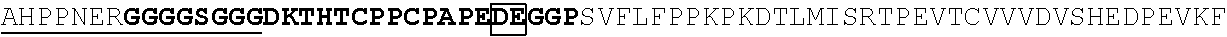

NWYVDGVEVHNAKTKPREEQYNSTYRVVSVLTVLHQDWLNGKEYKCKVSNKALPAPIEKTISKAKGQPR

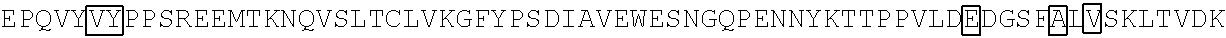

SRWQQGNVFSCSVMHEALHNHYTQKSLSLSPGK
(human PD-L1 extracellular domain mutant7 I54Q/E58M/R113T/M115L/S117A
extracellular domain-linker-hinge-IgG1 Fc mutant1; PD-L1 is underlined; linker is bolded and underlined;
hinge is bolded;
SEQ ID NO: 282

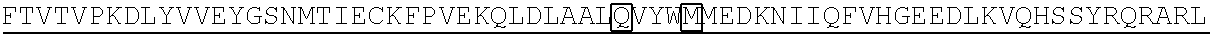

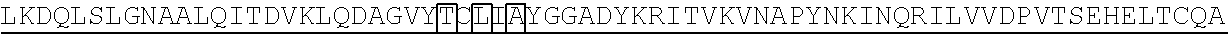

EGYPKAEVIWTSSDHQVLSGKTTTTNSKREEKLFNVTSTLRINTTTNEIFYCTFRRLDPEENHTAELVIPELPL

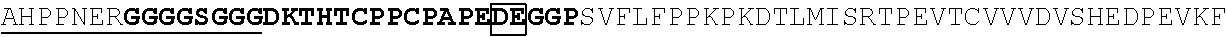

NWYVDGVEVHNAKTKPREEQYNSTYRVVSVLTVLHQDWLNGKEYKCKVSNKALPAPIEKTISKAKGQPR

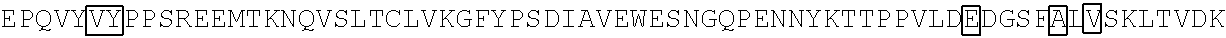

SRWQQGNVFSCSVMHEALHNHYTQKSLSLSPGK
(human PD-L1 extracellular domain mutant2 154Q/E58M/R113T/M115L/S117A/G119K
extracellular domain-linker-hinge-IgG1 Fc mutant2; PD-L1 is underlined; linker is bolded and underlined;
hinge is bolded;
SEQ ID NO: 283

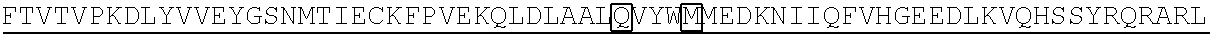

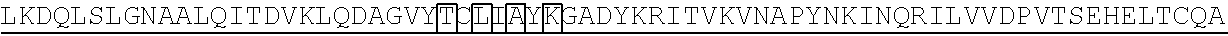

EGYPKAEVIWTSSDHQVLSGKTTTTNSKREEKLFNVTSTLRINTTTNEIFYCTFRRLDPEENHTAELVIPELPL

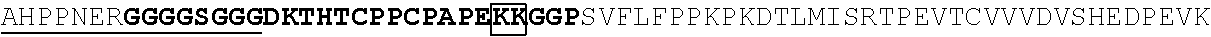

FNWYVDGVEVHNAKTKPREEQYNSTYRVVSVLTVLHQDWLNGKEYKCKVSNKALPAPIEKTISKAKGQP

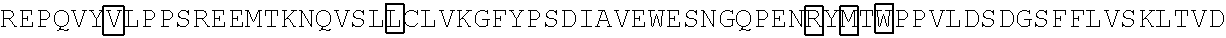

KSRWQQGNVFSCSVMHEALHNHYTQKSLSLSPGK
(human PD-L1 extracellular domain mutant7 154Q/E58M/R113T/M115L/S117A
extracellular domain-linker- hinge-IgG1 Fc mutant2; PD-L1 is underlined; linker is bolded and underlined;
hinge is bolded;
SEQ ID NO: 284

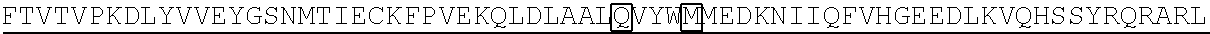

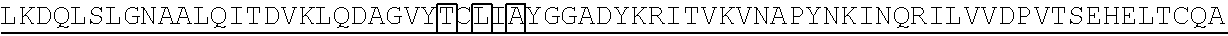

EGYPKAEVIWTSSDHQVLSGKTTTTNSKREEKLFNVTSTLRINTTTNEIFYCTFRRLDPEENHTAELVIPELPL

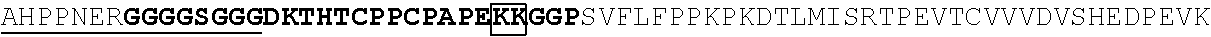

FNWYVDGVEVHNAKTKPREEQYNSTYRVVSVLTVLHQDWLNGKEYKCKVSNKALPAPIEKTISKAKGQP

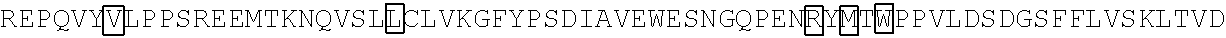

KSRWQQGNVFSCSVMHEALHNHYTQKSLSLSPGK
(human PD-L1 extracellular domain mutant2 154Q/E58M/R113T/M115L/S117A/G119K
extracellular domain-linker- IL-2 mutant L18R/Q22E/R38D/K43E/E61R)-linker-hinge-IgG1 Fc mutant2;
PD-L1 is underlined; linker is bolded and underlined; hinge is bolded; IL-12 subunits are italicized
SEQ ID NO: 285

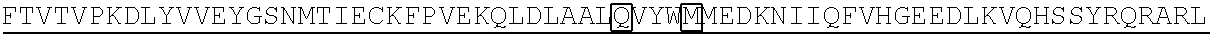

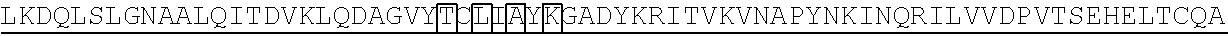

EGYPKAEVIWTSSDHQVLSGKTTTTNSKREEKLFNVTSTLRINTTTNEIFYCTFRRLDPEENHTAELVIPELPL

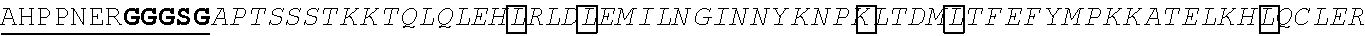

ELKPLEEVLNLAQSKNFHLRPRDLISNINVIVLELKGSETTFMCEYADETATIVEFLNRWITFCOSIISTLT

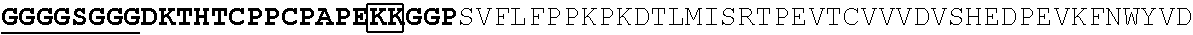

GVEVHNAKTKPREEQYNSTYRVVSVLTVLHQDWLNGKEYKCKVSNKALPAPIEKTISKAKGQPREPQVY

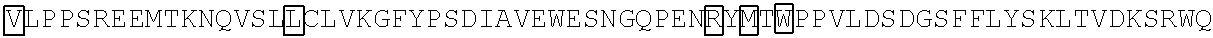

QGNVFSCSVMHEALHNHYTQKSLSLSPGK
(human PD-L1 extracellular domain mutant2 I54Q/E58M/R113T/M115L/S117A/G119K
extracellular domain-linker- IL-2 mutant R38D/K43E/E61R/Q126T)-linker-hinge-IgG1 Fc mutant2; PD-L1
is underlined; linker is bolded and underlined; hinge is bolded; IL-12 subunits are italicized
SEQ ID NO: 286

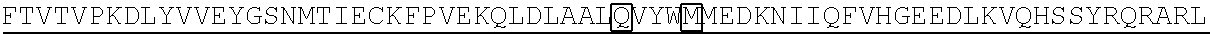

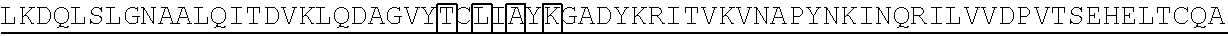

EGYPKAEVIWTSSDHQVLSGKTTTTNSKREEKLFNVTSTLRINTTTNEIFYCTFRRLDPEENHTAELVIPELPL

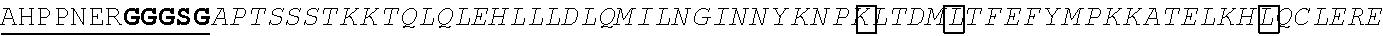

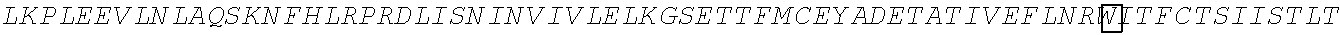

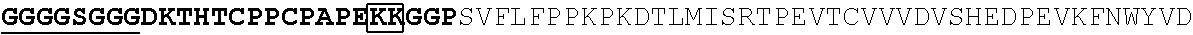

GVEVHNAKTKPREEQYNSTYRVVSVLTVLHQDWLNGKEYKCKVSNKALPAPIEKTISKAKGQPREPQVY

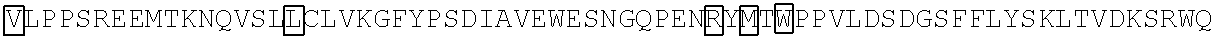

QGNVFSCSVMHEALHNHYTQKSLSLSPGK
(human PD-L1 extracellular domain mutant2 154Q/E58M/R113T/M115L/S117A/G119K
extracellular domain-linker-IL-2 mutant L18R/Q22E/R38D/K43E/E61R/Q126T)-linker-hinge-IgG1 Fc
mutant2; PD-L1 is underlined; linker is bolded and underlined; hinge is bolded; IL-12 subunits are italicized
SEQ ID NO: 287

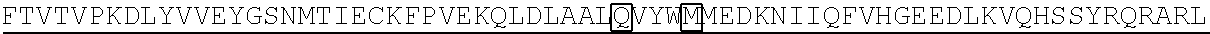

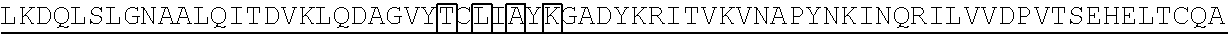

EGYPKAEVIWTSSDHQVLSGKTTTTNSKREEKLFNVTSTLRINTTTNEIFYCTFRRLDPEENHTAELVIPELPL

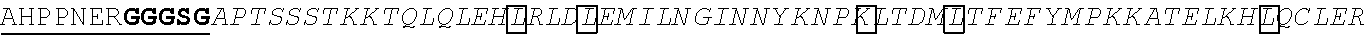

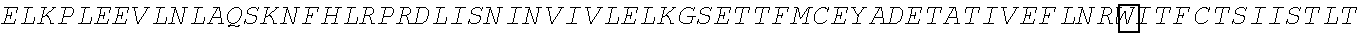

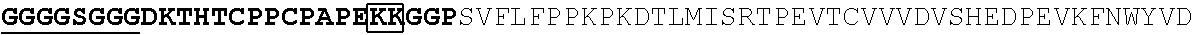

GVEVHNAKTKPREEQYNSTYRVVSVLTVLHQDWLNGKEYKCKVSNKALPAPIEKTISKAKGQPREPQVY

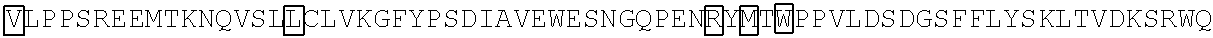

QGNVFSCSVMHEALHNHYTQKSLSLSPGK
(human PD-L1 extracellular domain mutant2 154Q/E58M/R113T/M115L/S117A/G119K
extracellular domain-linker- IL-2 mutant L18R/Q22E/R38D/K43E/E61R/Q126T/S130R)-linker-hinge-IgG1
Fc mutant2; PD-L1 is underlined; linker is bolded and underlined; hinge is bolded; IL-12 subunits are
italicized
SEQ ID NO: 288

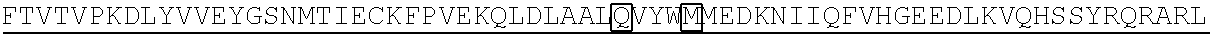

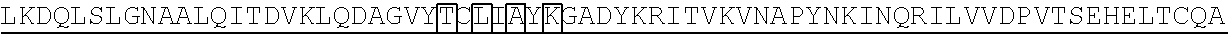

EGYPKAEVIWTSSDHQVLSGKTTTTNSKREEKLFNVTSTLRINTTTNEIFYCTFRRLDPEENHTAELVIPELPL

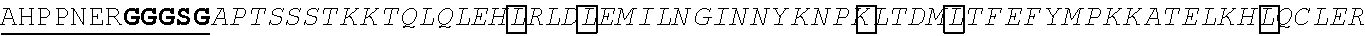

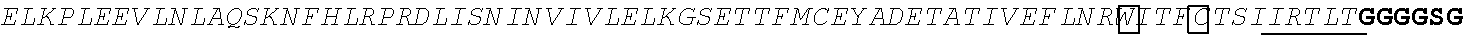

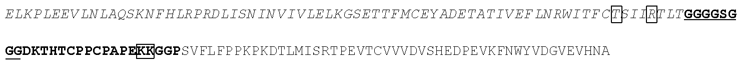

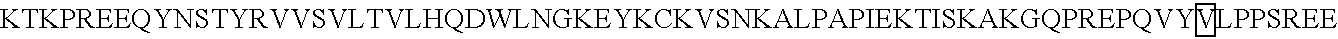

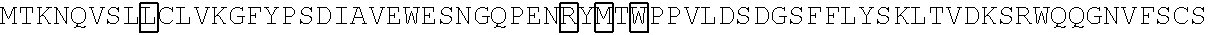

VMHEALHNHYTQKSLSLSPGK
(human PD-L1 extracellular domain mutant7 154Q/E58M/R113T/M115L/S117A/G119K
extracellular domain-linker-IL-2 mutant L18R/Q22E/R38D/K43E/E61R)-linker-hinge-IgG1 Fc mutant2; PD-
L1 is underlined; linker is bolded and underlined; hinge is bolded; IL-12 subunits are italicized
SEQ ID NO: 289

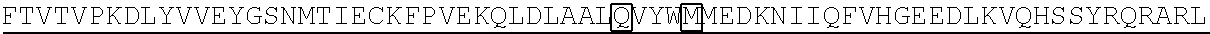

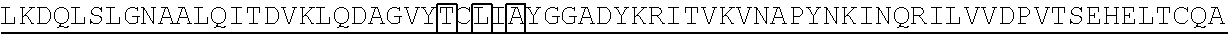

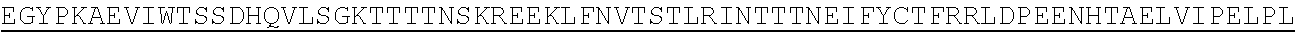

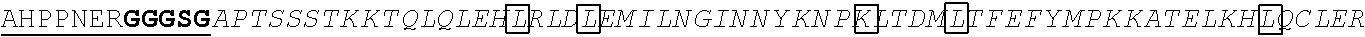

ELKPLEEVLNLAOSKNFHLRPRDLISNINVIVLELKGSETTFMCEYADETATIVEFLNRWITFCQSIISTLT

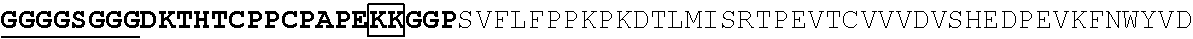

GVEVHNAKTKPREEQYNSTYRVVSVLTVLHQDWLNGKEYKCKVSNKALPAPIEKTISKAKGQPREPQVY

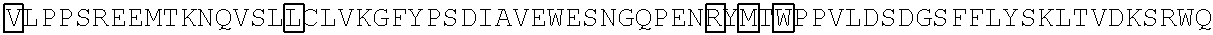

QGNVFSCSVMHEALHNHYTQKSLSLSPGK
(human PD-L1 extracellular domain mutant7 154Q/E58M/R113T/M115L/S117A
extracellular domain-linker-IL-2 mutant R38D/K43E/E61R/Q126T)-linker-hinge-IgG1 Fc mutant2; PD-L1 is
underlined; linker is bolded and underlined; hinge is bolded; IL-12 subunits are italicized
SEQ ID NO: 290

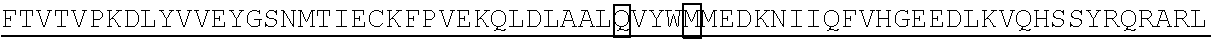

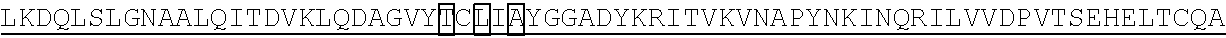

EGYPKAEVIWTSSDHQVLSGKTTTTNSKREEKLFNVTSTLRINTTTNEIFYCTFRRLDPEENHTAELVIPELPL

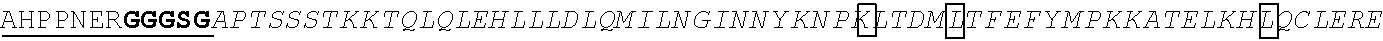

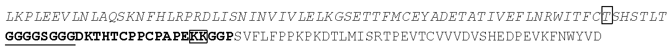

GVEVHNAKTKPREEQYNSTYRVVSVLTVLHQDWLNGKEYKCKVSNKALPAPIEKTISKAKGQPREPQVY

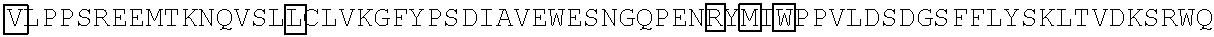

QGNVFSCSVMHEALHNHYTQKSLSLSPGK
(human PD-L1 extracellular domain mutant7 154Q/E58M/R113T/M115L/S117A
extracellular domain-linker- IL-2 mutant L18R/Q22E/R38D/K43E/E61R/Q126T)-linker-hinge-IgG1 Fc
mutant2; PD-L1 is underlined; linker is bolded and underlined; hinge is bolded; IL-12 subunits
are italicized
SEQ ID NO: 291

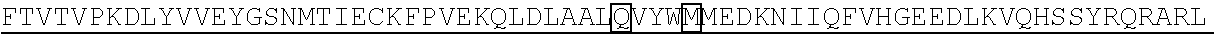

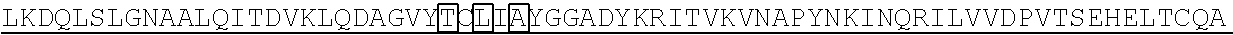

EGYPKAEVIWTSSDHQVLSGKTTTTNSKREEKLFNVTSTLRINTTTNEIFYCTFRRLDPEENHTAELVIPELPL

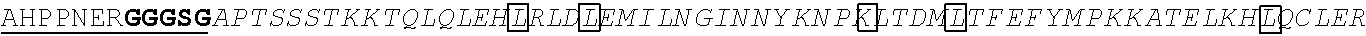

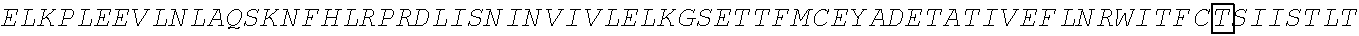

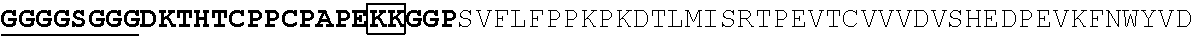

GVEVHNAKTKPREEQYNSTYRVVSVLTVLHQDWLNGKEYKCKVSNKALPAPIEKTISKAKGQPREPQVY

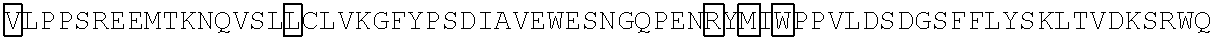

QGNVFSCSVMHEALHNHYTQKSLSLSPGK
(human PD-L1 extracellular domain mutant7 154Q/E58M/R113T/M115L/S117A
extracellular domain-linker-IL-2 mutant L18R/Q22E/R38D/K43E/E61R/Q126T/S130R)-linker-hinge-IgG1
Fc mutant2; PD-L1 is underlined; linker is bolded and underlined; hinge is bolded; IL-12 subunits are
italicized
SEQ ID NO: 292

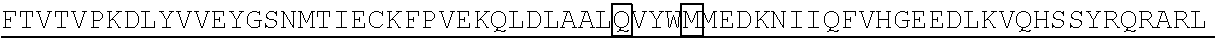

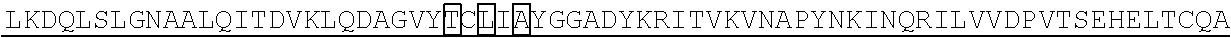

EGYPKAEVIWTSSDHQVLSGKTTTTNSKREEKLFNVTSTLRINTTTNEIFYCTFRRLDPEENHTAELVIPELPL

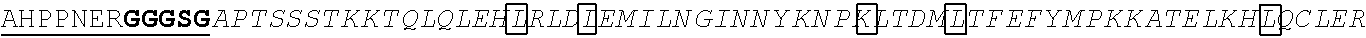

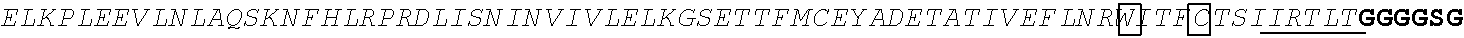

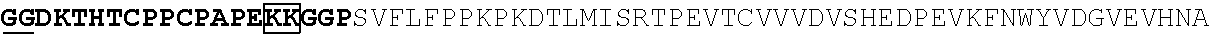

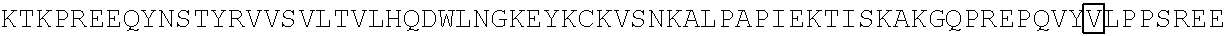

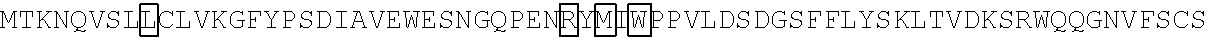

VMHEALHNHYTQKSLSLSPGK
(human PD-L1 extracellular domain mutant7 154Q/E58M/R113T/M115L/S117A
extracellular domain-linker- IL-2 mutant L18R/Q22E/R38D/K43E/E61R/Q126T)-linker-hinge-IgG1 Fc
mutant2; PD-L1 is underlined; linker is bolded and underlined; hinge is bolded; IL-12 subunits
are italicized
SEQ ID NO: 291

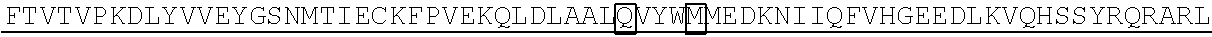

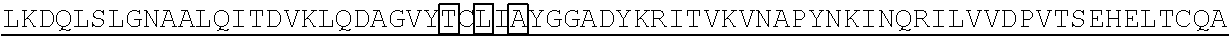

EGYPKAEVIWTSSDHQVLSGKTTTTNSKREEKLFNVTSTLRINTTTNEIFYCTFRRLDPEENHTAELVIPELPL

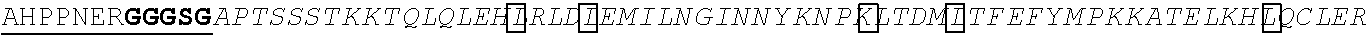

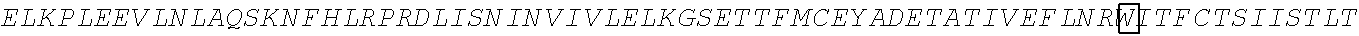

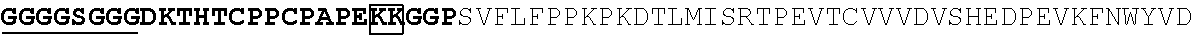

GVEVHNAKTKPREEQYNSTYRVVSVLTVLHQDWLNGKEYKCKVSNKALPAPIEKTISKAKGQPREPQVY

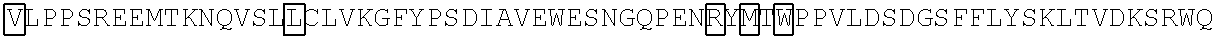

QGNVFSCSVMHEALHNHYTQKSLSLSPGK
(human PD-L1 extracellular domain mutant7 I54Q/E58M/R113T/M115L/S117A
extracellular domain-linker-IL-2 mutant L18R/Q22E/R38D/K43E/E61RQ126T/S130R)-linker-hinge-IgG1 Fc
mutant2; PD-L1 is underlined; linker is bolded and underlined; hinge is bolded; IL-12 subunits
are italicized
SEQ ID NO: 292

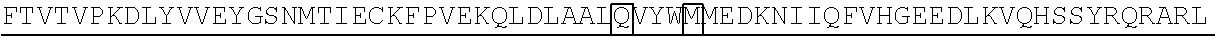

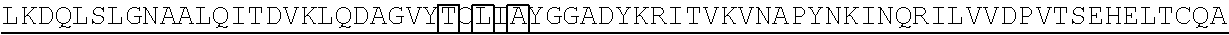

EGYPKAEVIWTSSDHQVLSGKTTTTNSKREEKLFNVTSTLRINTTTNEIFYCTFRRLDPEENHTAELVIPELPL

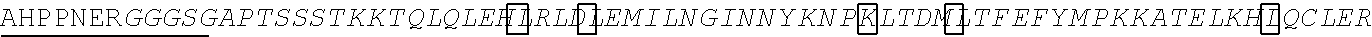

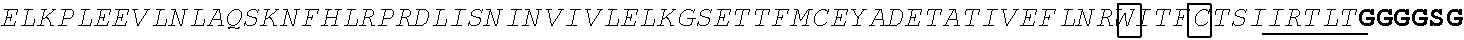

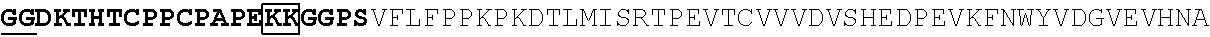

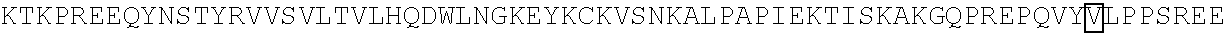

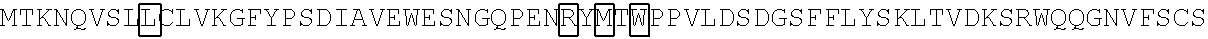

VMHEALHNHYTQKSLSLSPGK
(human PD-L2 extracellular domain mutant2 S58V extracellular domain-linker-single-
chain IL-12 mutant heterodimer IL-12B (p40 E59A/F60A)-linker IL-12A (wt p35)-hinge-IgG1 Fc mutant1;
PD-L2 is underlined; linker is bolded and underlined; hinge is bolded; IL-12 subunits are italicized)
SEQ ID NO: 293

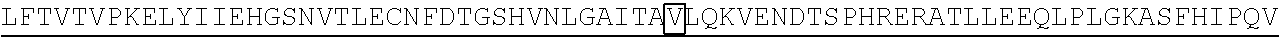

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGIWELKK

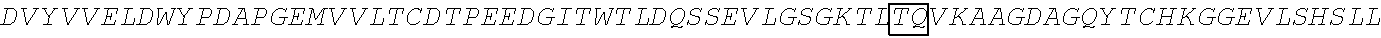

LLHKKEDGIWSTDILKDOKEPKNKTFLRCEAKNYSGRFTCWWLTTISTDLTFSVKSSRGSSDPOGVTCGAATLSAE
RVRGDNKEYEYSVECQEDSACPAAEESLPIEVMVDAVHKLKYENYTSSFFIRDIIKPDPPKNLOLKPLKNSRQVEVS
WEYPDTWSTPHSYFSLTFCVQVQGKSKREKKDRVFTDKTSATVICRKNASISVRAQDRYYSSSWSEWASVPCSGGG
GSGGGSGGGGSRNLPVATPDPGMFPCLHHSONLLRAVSNMLQKARQTLEFYPCTSEEIDHEDITKDKTSTVEAC
LPLELTKNESCLNSRETSFITNGSCLASRKTSFMMALCLSSIYEDLKMYQVEFKTMNAKLLMDPKRQIFLDQNMLA
VIDELMOALNFNSETVPQKSSLEEPDFYKTKIKLCILLHAFRIRAVTIDRVMSYLNASGGGGSGGGDKTHTCPPC

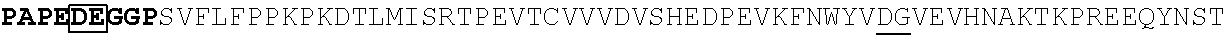

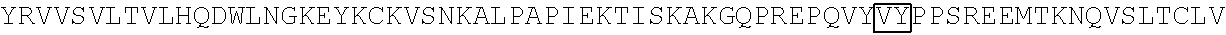

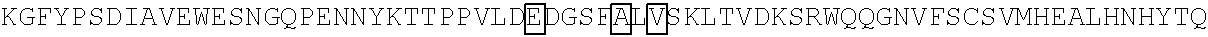

KSLSLSPGK
(human PD-L2 extracellular domain mutant4 T56V/S58V/Q60L extracellular domain-
linker-single-chain IL-12 mutant heterodimer IL-12B (p40 E59A/F60A)-linker IL-12A (wt p35)-hinge-IgG1
Fc mutant1; PD-L2 is underlined; linker is bolded and underlined; hinge is bolded; IL-12 subunits are
italicized)
SEQ ID NO: 294

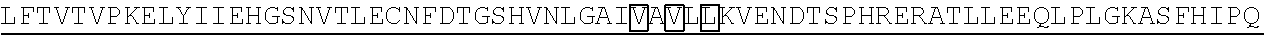

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGIWELK

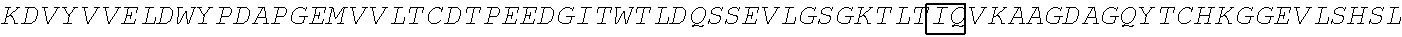

LLLHKKEDGIWSTDILKDOKEPKNKTFLRCEAKNYSGRFTCWWLTTISTDLTFSVKSSRGSSDPQGVTCGAATLSA
ERVRGDNKEYEYSVECOEDSACPAAEESLPIEVMVDAVHKLKYENYTSSFFIRDIIKPDPPKNLOLKPLKNSRQVEV
SWEYPDTWSTPHSYFSLTFCVQVQGKSKREKKDRVFTDKTSATVICRKNASISVRAQDRYYSSSWSEWASVPCSGG
GGSGGGSGGGGSRNLPVATPDPGMFPCLHHSQNLLRAVSNMLQKARQTLEFYPCTSEEIDHEDITKDKTSTVE
ACLPLELTKNESCLNSRETSFITNGSCLASRKTSFMMALCLSSIYEDLKMYQVEFKTMNAKLLMDPKRQIFLDQNM
LAVIDELMQALNFNSETVPQKSSLEEPDFYKTKIKLCILLHAFRIRAVTIDRVMSYLNASGGGGSGGGDKTHTCP

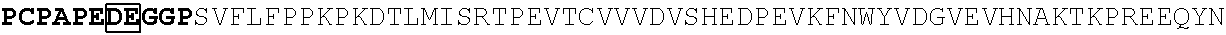

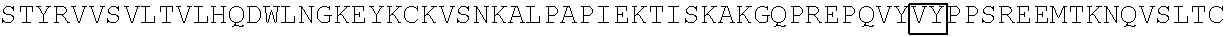

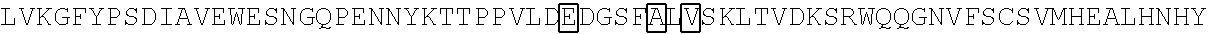

TQKSLSLSPGK
(human PD-L2 extracellular domain mutant2 S58V extracellular domain-linker-hinge-IgG1
Fc mutant1-linker-single-chain IL-12 mutant heterodimer IL-12B (p40 E59A/F60A)-linker IL-12A (wt p35);
PD-L2 is underlined; linker is bolded and underlined; hinge is bolded; IL-12 subunits are italicized)
SEQ ID NO: 295

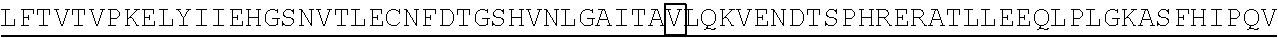

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGDKTHT

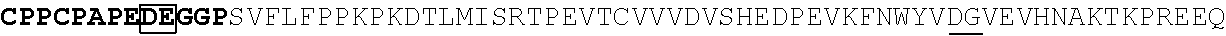

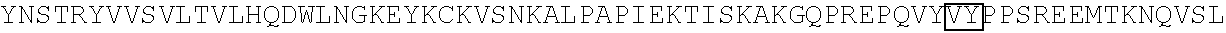

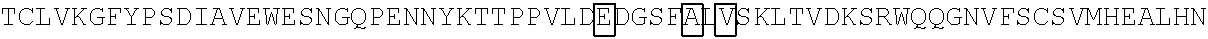

HYTQKSLSLSPGKGGGGSGGGIWELKKDVYVVELDWYPDAPGEMVVLTCDTPEEDGITWTLDQSSEVLGSGK

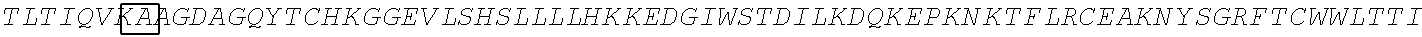

STDLTFSVKSSRGSSDPQGVTCGAATLSAERVRGDNKEYEYSVECQEDSACPAAEESLPIEVMVDAVHKLKYENYT
SSFFIRDIIKPDPPKNLOLKPLKNSRQVEVSWEYPDTWSTPHSYFSLTFCVQVQGKSKREKKDRVFTDKTSATVICR
KNASISVRAQDRYYSSSWSEWASVPCSGGGGSGGGSGGGGSRNLPVATPDPGMFPCLHHSQNLLRAVSNMLQK
ARQTLEFYPCTSEEIDHEDITKDKTSTVEACLPLELTKNESCLNSRETSFITNGSCLASRKTSFMMALCLSSIYEDLK
MYQVEFKTMNAKLLMDPKRQIFLDQNMLAVIDELMQALNFNSETVPQKSSLEEPDFYKTKIKLCILLHAFRIRAVT
IDRVMSYLNAS
(human PD-L2 extracellular domain mutant4 T56V/S58V/Q60L extracellular domain-
linker- hinge-IgG1 Fc mutant1- linker-single-chain IL-12 mutant heterodimer IL-12B (p40 E59A/F60A)-
linker IL-12A (wt p35); PD-L2 is underlined; linker is bolded and underlined; hinge is bolded;
IL-12 subunits are italicized)
SEQ ID NO: 296

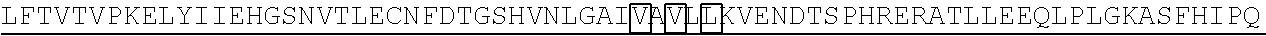

VQVRDEGQYQCIIIYGVAWDYKYLTLKVKASYRKINTHILKVPETDEVELTCOATGYPLAEVSWPNVSVPA
NTSHSRTPEGLYQVTSVLRLKPPPGRNFSCVFWNTHVRELTLASIDLQSQMEPRTHPTGGGGSGGGDKTH

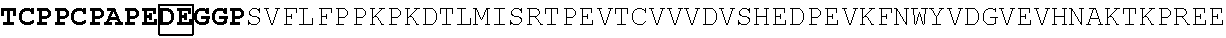

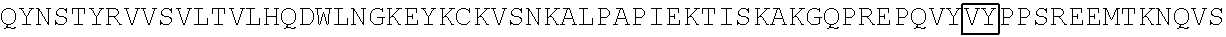

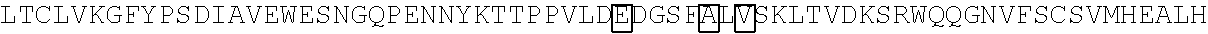

NHYTQKSLSLSPGKGGGGSGGGIWELKKDVYVVELDWYPDAPGEMVVLTCDTPEEDGITWTLDOSSEVLGSG

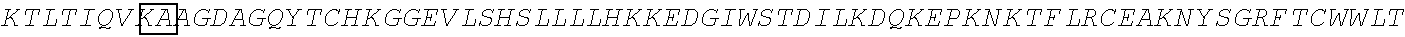

TISTDLTFSVKSSRGSSDPOGVTCGAATLSAERVRGDNKEYEYSVECOEDSACPAAEESLPIEVMVDAVHKLKYENY
TSSFFIRDIIKPDPPKNLOLKPLKNSROVEVSWEYPDTWSTPHSYFSLTFCVOVOGKSKREKKDRVFTDKTSATVIC
RKNASISVRAQDRYYSSSWSEWASVPCSGGGGSGGGSGGGGSRNLPVATPDPGMFPCLHHSQNLLRAVSNMLQ
KARQTLEFYPCTSEEIDHEDITKDKTSTVEACLPLELTKNESCLNSRETSFITNGSCLASRKTSFMMALCLSSIYEDL
KMYQVEFKTMNAKLLMDPKRQIFLDQNMLAVIDELMQALNFNSETVPQKSSLEEPDFYKTKIKLCILLHAFRIRA
VTIDRVMSYLNAS
(human PD-L2 extracellular domain mutant2 S58V extracellular domain-linker-hinge-IgG1
Fc mutant1; PD-L2 is underlined; linker is bolded and underlined; hinge is bolded;
SEQ ID NO: 297

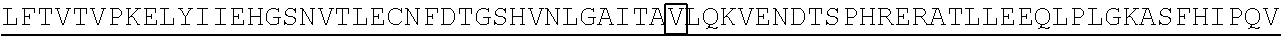

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGDKTHT

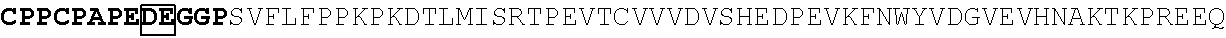

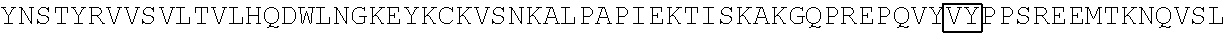

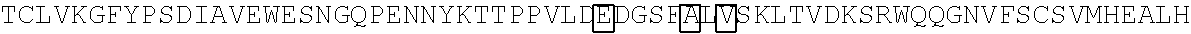

HYTQKSLSLSPGK
(human PD-L2 extracellular domain mutant4 T56V/S58V/Q60L extracellular domain-
linker-hinge-IgG1 Fc mutant1; PD-L2 is underlined; linker is bolded and underlined; hinge is bolded;
SEQ ID NO: 298

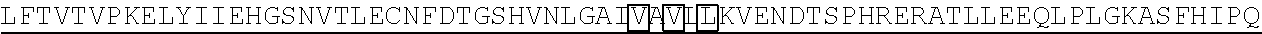

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGDKTH

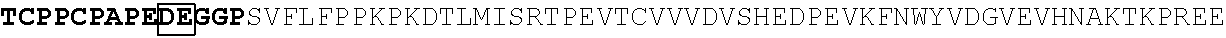

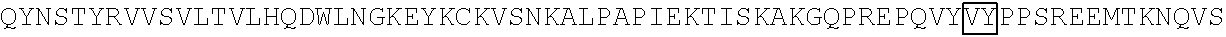

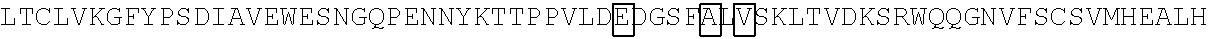

NHYTQKSLSLSPGK
(human PD-L2 extracellular domain mutant2 S58V extracellular domain-linker-hinge-IgG1
Fc mutant2; PD-L2 is underlined; linker is bolded and underlined; hinge is bolded;
SEQ ID NO: 299

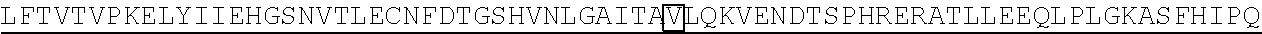

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGDKTHT

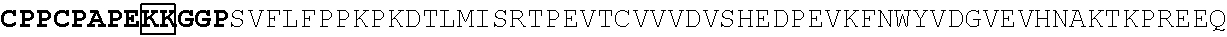

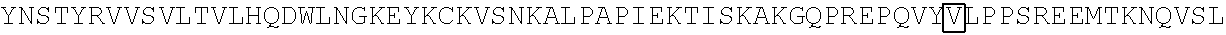

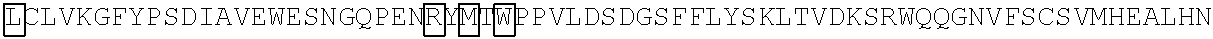

HYTQKSLSLSPGK
(human PD-L2 mutant4 T56V/S58V/Q60L extracellular domain-linker- hinge-IgG1 Fc
mutant2; PD-L2 is underlined; linker is bolded and underlined; hinge is bolded;
SEQ ID NO: 300

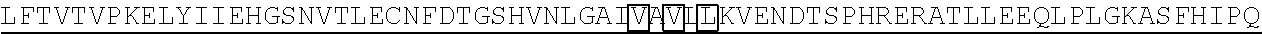

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGDKTH

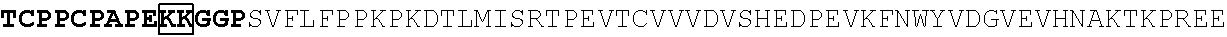

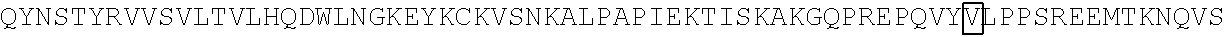

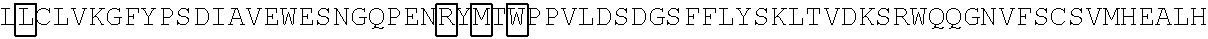

NHYTQKSLSLSPGK
(human PD-L2 extracellular domain mutant2 S58V extracellular domain-linker-IL-2
mutant L18R/Q22E/R38D/K43E/E61R)-linker-hinge-IgG1 Fc mutant2; PD-L2 is underlined; linker is bolded
and underlined; hinge is bolded; IL-12 subunits are italicized
SEQ ID NO: 301

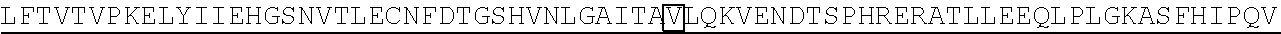

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGSGAPTSSSTKKT

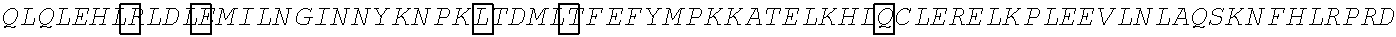

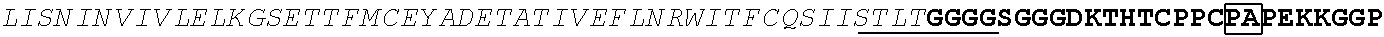

SVFLFPPKPKDTLMISRTPEVTCVVVDVSHEDPEVKFNWYVDGVEVHNAKTKPREEQYNSTYRVVSVLTV

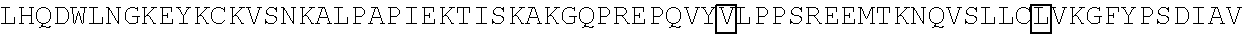

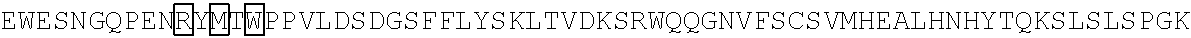

(human PD-L2 extracellular domain mutant2 S58V extracellular domain-linker-IL-2
mutant R38D/K43E/E61R/Q126T)-linker-hinge-IgG1 Fc mutant2; PD-L2 is underlined; linker is bolded and
underlined; hinge is bolded; IL-12 subunits are italicized
SEQ ID NO: 302

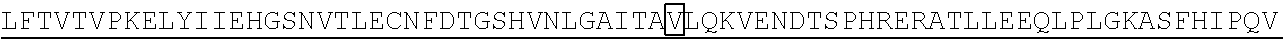

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNFSCVFWNTHVRELTLASIDLQSQMEPRTHPTGGGSGAPTSSSTKKTQL

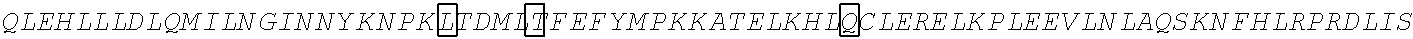

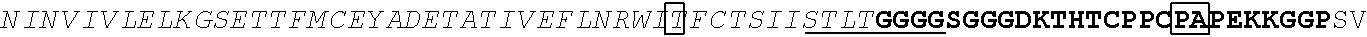

FLFPPKPKDTLMISRTPEVTCVVVDVSHEDPEVKFNWYVDGVEVHNAKTKPREEQYNSTYRVVSVLTVLH

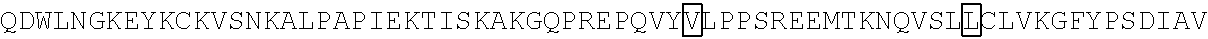

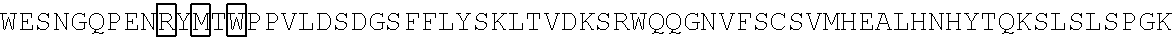

(human PD-L2 extracellular domain mutant2 S58V extracellular domain-linker-IL-2
mutant L18R/Q22E/R38D/K43E/E61R/Q126T)-linker-hinge-IgG1 Fc mutant2; PD-L2 is underlined; linker is
bolded and underlined; hinge is bolded; IL-12 subunits are italicized
SEQ ID NO: 303

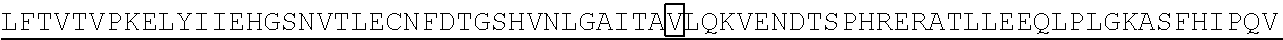

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNFSCVFWNTHVRELTLASIDLQSQMEPRTHPTGGGSGAPTSSSTKKTQL

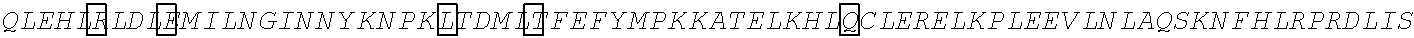

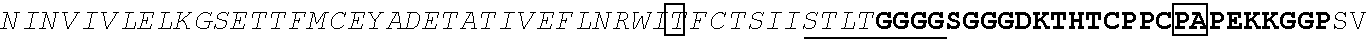

FLFPPKPKDTLMISRTPEVTCVVVDVSHEDPEVKFNWYVDGVEVHNAKTKPREEQYNSTYRVVSVLTVLH

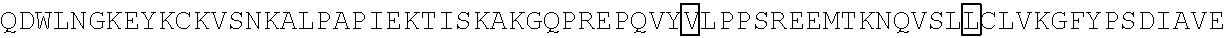

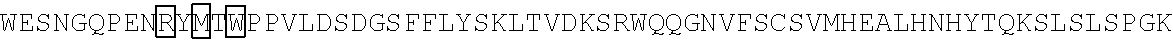

(human PD-L2 extracellular domain mutant2 S58V extracellular domain-linker-IL-2
mutant R38D/K43E/E61R)(L18R/Q22E/Q126T/S130R)-linker-hinge-IgG1 Fc mutant2; PD-L2 is underlined;
linker is bolded and underlined; hinge is bolded; IL-12 subunits are italicized
SEQ ID NO: 304

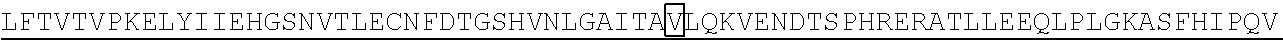

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNFSCVFWNTHVRELTLASIDLQSQMEPRTHPTGGGSGAPTSSSTKKTQL

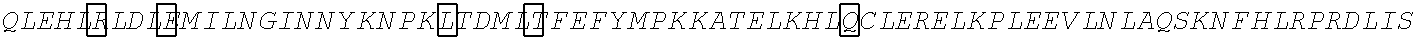

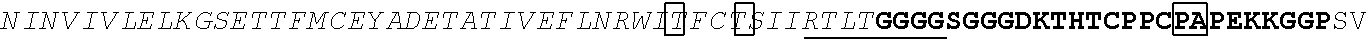

FLFPPKPKDTLMISRTPEVTCVVVDVSHEDPEVKFNWYVDGVEVHNAKTKPREEQYNSTYRVVSVLTVLH

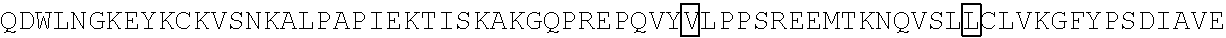

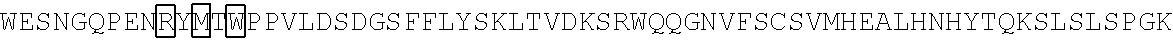

PPVLDSDGSFFLYSKLTVDKSRWQQGNVFSCSVMHEALHNHYTQKSLSLSPGK
(human PD-L2 extracellular domain mutant4 T56V/S58V/Q60L extracellular domain-
linker-IL-2 mutant L18R/Q22E/R38D/K43E/E61R)-linker-hinge-IgG1 Fc mutant2; PD-L2 is underlined;
linker is bolded and underlined; hinge is bolded; IL-12 subunits are italicized
SEQ ID NO: 305

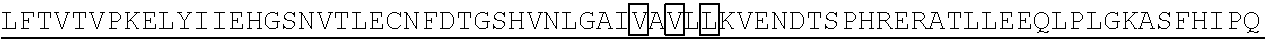

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

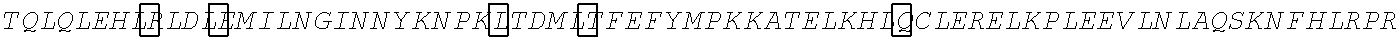

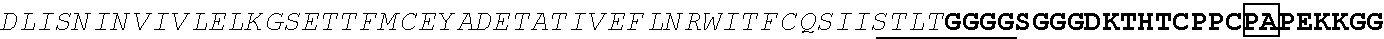

PSVFLFPPKPKDTLMISRTPEVTCVVVDVSHEDPEVKFNWYVDGVEVHNAKTKPREEQYNSTYRVVSVLT

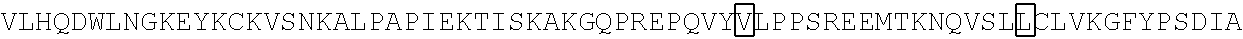

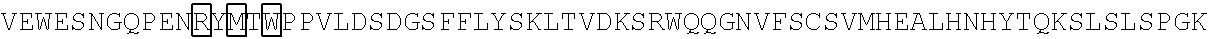

(human PD-L2 extracellular domain mutant4 T56V/S58V/Q60L extracellular domain-
linker-IL-2 mutant R38D/K43E/E61R/Q126T)-linker-hinge-IgG1 Fc mutant2; PD-L2 is underlined; linker is
bolded and underlined; hinge is bolded; IL-12 subunits are italicized
SEQ ID NO: 306

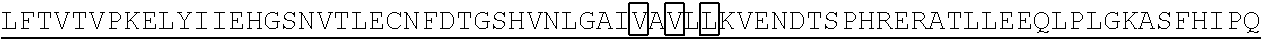

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

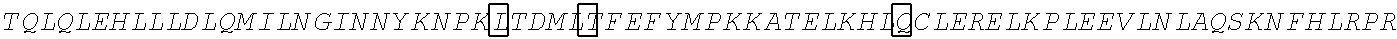

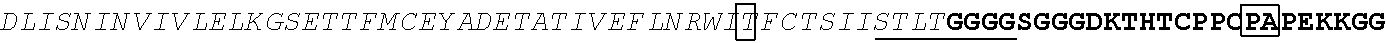

PSVFLFPPKPKDTLMISRTPEVTCVVVDVSHEDPEVKFNWYVDGVEVHNAKTKPREEQYNSTYRVVSVLT

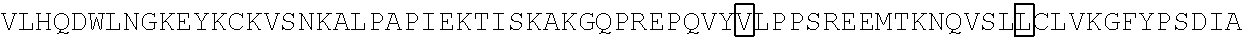

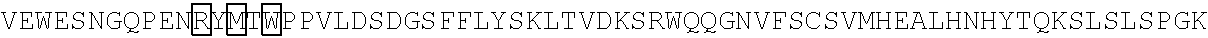

(human PD-L2 mutant4 T56V/S58V/Q60L extracellular domain-linker-IL-2 mutant
L18R/Q22E/R38D/K43E/E61R/Q126T)-linker-hinge-IgG1 Fc mutant2; PD-L2 is underlined; linker is bolded
and underlined; hinge is bolded; IL-12 subunits are italicized
SEQ ID NO: 307

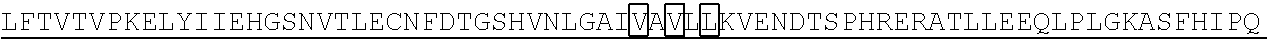

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

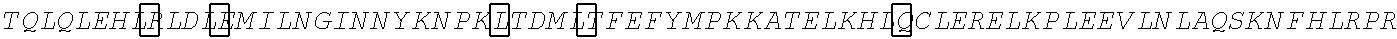

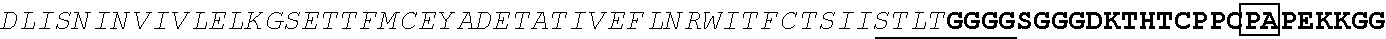

PSVFLFPPKPKDTLMISRTPEVTCVVVDVSHEDPEVKFNWYVDGVEVHNAKTKPREEQYNSTYRVVSVLT

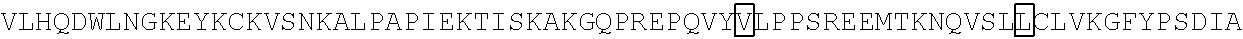

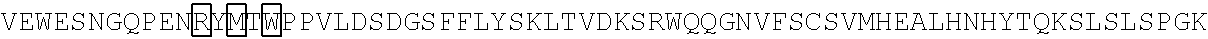

(human PD-L2 mutant4 T56V/S58V/Q60L extracellular domain-linker-IL-2 mutant
L18R/Q22E/R38D/K43E/E61R/Q126T/S130R)-linker-hinge-IgG1 Fc mutant2; PD-L2 is underlined; linker is
bolded and underlined; hinge is bolded; IL-12 subunits are italicized
SEQ ID NO: 308

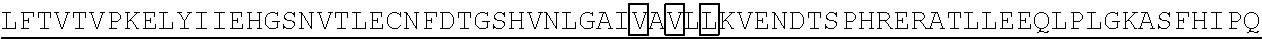

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

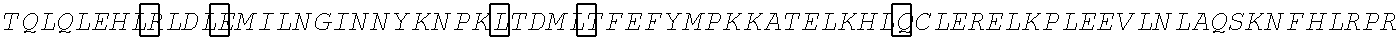

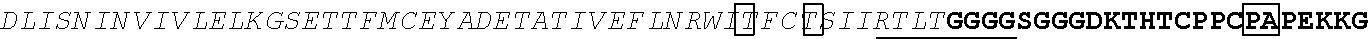

GPSVFLFPPKPKDTLMISRTPEVTCVVVDVSHEDPEVKFNWYVDGVEVHNAKTKPREEQYNSTYRVVSVL

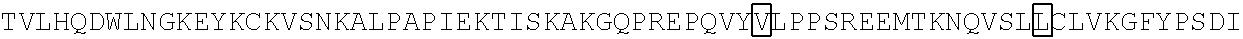

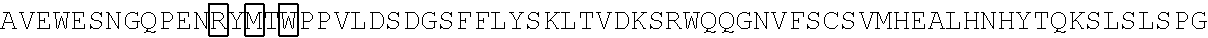

K
(human CD155 extracellular domain-linker-single-chain IL-12 mutant heterodimer IL-12B
(p40 E59A/F60A)-linker IL-12A (wt p35)-hinge-IgG1 Fc mutant1; CD155 is underlined; linker is bolded and
underlined; hinge is bolded; IL-12 subunits are italicized)
SEQ ID NO: 309
WPPPGTGDVVVQAPTQVPGFLGDSVTLPCYLQVPNMEVTHVSQLTWARHGESGSMAVFHQTOGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRNGGGGSGGGIWELKKDVYVVELDWYPDAPG

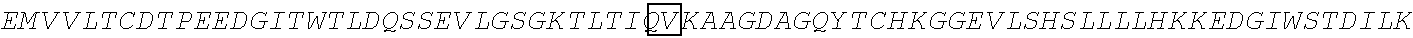

DQKEPKNKTFLRCEAKNYSGRFTCWWLTTISTDLTFSVKSSRGSSDPOGVTCGAATLSAERVRGDNKEYEYSVEC
QEDSACPAAEESLPIEVMVDAVHKLKYENYTSSFFIRDIIKPDPPKNLOLKPLKNSRQVEVSWEYPDTWSTPHSYFS
LTFCVQVQGKSKREKKDRVFTDKTSATVICRKNASISVRAQDRYYSSSWSEWASVPCSGGGGSGGGSGGGGSRN
LPVATPDPGMFPCLHHSONLLRAVSNMLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPKRQIFLDONMLAVIDELMQALNENSE

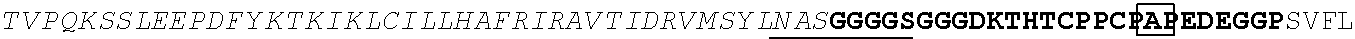

FPPKPKDTLMISRTPEVTCVVVDVSHEDPEVKFNWYVDGVEVHNAKTKPREEQYNSTYRVVSVLTVLHQD

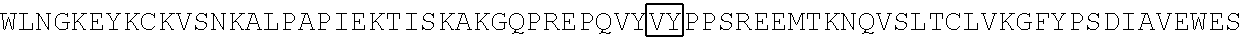

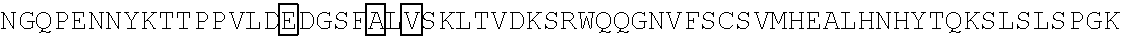

(human CD155 extracellular domain-linker-hinge-IgG1 Fc mutant1-linker-single-chain IL-
12 mutant heterodimer IL-12B (p40 E59A/F60A)-linker IL-12A (wt p35); CD155 is underlined; linker is
bolded and underlined; hinge is bolded; IL-12 subunits are italicized)
SEQ ID NO: 310
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPONTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRN

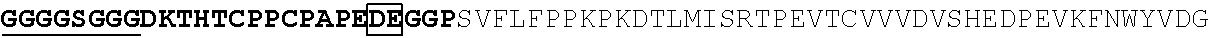

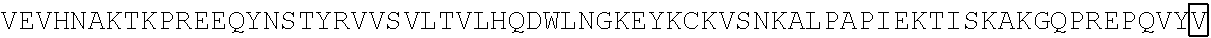

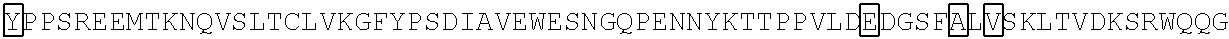

NVFSCSVMHEALHNHYTQKSLSLSPGKGGGGSGGGIWELKKDVYVVELDWYPDAPGEMVVLTCDTPEEDGI

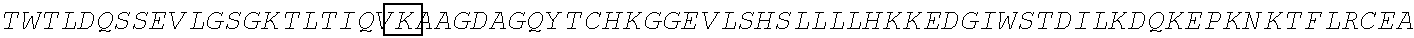

KNYSGRFTCWWLTTISTDLTFSVKSSRGSSDPQGVTCGAATLSAERVRGDNKEYEYSVECQEDSACPAAEESLPIEV
MVDAVHKLKYENYTSSFFIRDIIKPDPPKNLOLKPLKNSROVEVSWEYPDTWSTPHSYFSLTFCVQVQGKSKREKK
DRVFTDKTSATVICRKNASISVRAQDRYYSSSWSEWASVPCSGGGGSGGGSGGGGSRNLPVATPDPGMFPCLHH
SQNLLRAVSNMLQKARQTLEFYPCTSEEIDHEDITKDKTSTVEACLPLELTKNESCLNSRETSFITNGSCLASRKTSF
MMALCLSSIYEDLKMYQVEFKTMNAKLLMDPKRQIFLDQNMLAVIDELMQALNFNSETVPQKSSLEEPDFYKTKI
KLCILLHAFRIRAVTIDRVMSYLNAS
(human CD155 extracellular domain-linker-hinge-IgG1 Fc mutant1; CD155 is underlined;
linker is bolded and underlined; hinge is bolded;
SEQ ID NO: 311
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

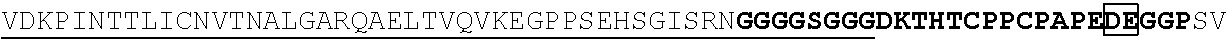

FLFPPKPKDTLMISRTPEVTCVVVDVSHEDPEVKFNWYVDGVEVHNAKTKPREEQYNSTYRVVSVLTVLH

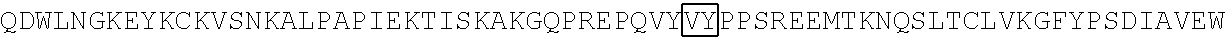

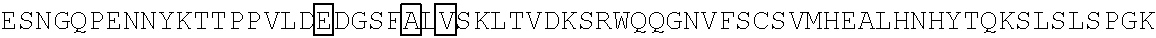

(human CD155 extracellular domain-linker-hinge-IgG1 Fc mutant2; CD155 is underlined;
linker is bolded and underlined; hinge is bolded;
SEQ ID NO: 312
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPONTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGONEATLTCDARSNPEPTGYNWSTTMGPLPPFAVAQGAQLLIRP

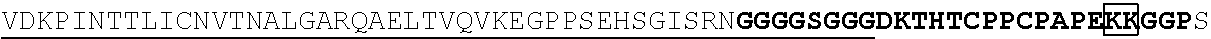

VFLFPPKPKDTLMISRTPEVTCVVVDVSHEDPEVKFNWYVDGVEVHNAKTKPREEQYNSTYRVVSVLTVL

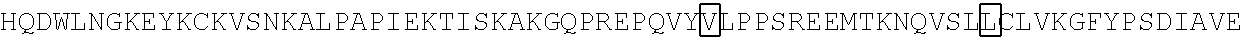

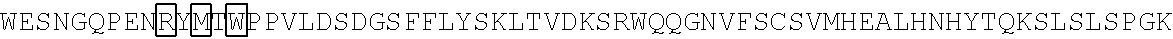

(human CD155 extracellular domain-linker- IL-2 mutant L18R/Q22E/R38D/K43E/E61R)-
linker-hinge-IgG1 Fc mutant2; CD155 is underlined; linker is bolded and underlined; hinge is bolded;
IL-12 subunits are italicized
SEQ ID NO: 313
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

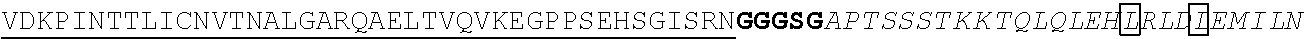

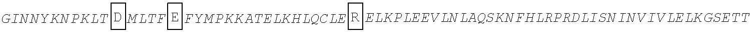

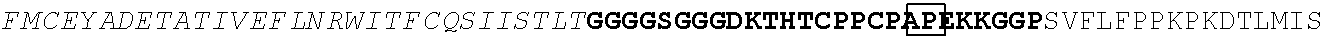

RTPEVTCVVVDVSHEDPEVKFNWYVDGVEVHNAKTKPREEQYNSTYRVVSVLTVLHQDWLNGKEYKCK

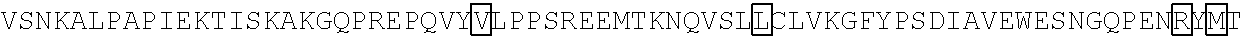

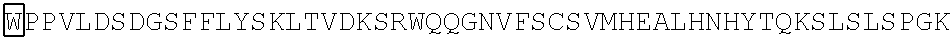

(human CD155 extracellular domain-linker-IL-2 mutant R38D/K43E/E61R/Q126T)-linker-
hinge-IgG1 Fc mutant2; CD155 is underlined; linker is bolded and underlined; hinge is bolded; IL-12
subunits are italicized
SEQ ID NO: 314
WPPPGTGDVVVQAPTQVPGFLGDSVTLPCYLQVPNMEVTHVSQLTWARHGESGSMAVFHQTOGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRNGGGSGAPTSSSTKKTQLQLEHLLLDLQMILN

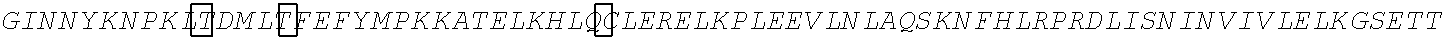

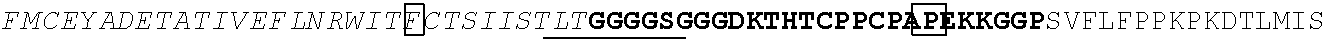

RTPEVTCVVVDVSHEDPEVKFNWYVDGVEVHNAKTKPREEQYNSTYRVVSVLTVLHQDWLNGKEYKCK

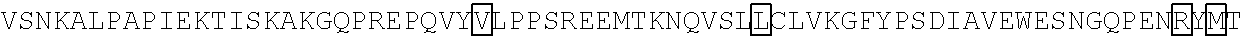

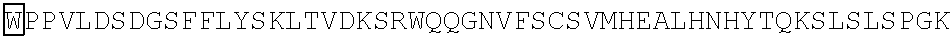

(human CD155 extracellular domain-linker-IL-2 mutant
L18R/Q22E/R38D/K43E/E61R/Q126T)-linker-hinge-IgG1 Fc mutant2; CD155 is underlined; linker is bolded
and underlined; hinge is bolded; IL-12 subunits are italicized
SEQ ID NO: 315
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPONTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

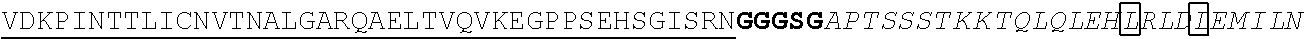

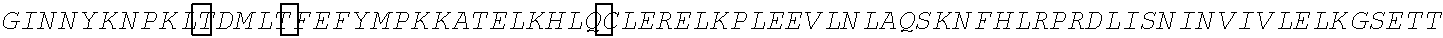

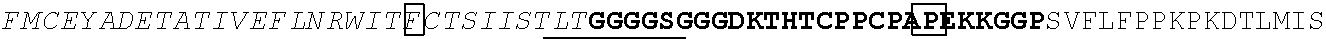

RTPEVTCVVVDVSHEDPEVKFNWYVDGVEVHNAKTKPREEQYNSTYRVVSVLTVLHQDWLNGKEYKCK

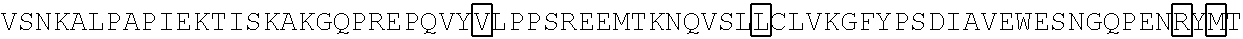

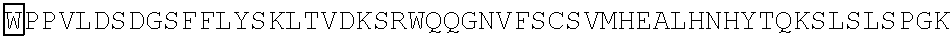

(human CD155 extracellular domain-linker- IL-2 mutant
L18R/Q22E/R38D/K43E/E61RQ126T/S130R)-linker-hinge-IgG1 Fc mutant2; CD155 is underlined; linker is
bolded and underlined; hinge is bolded; IL-12 subunits are italicized
SEQ ID NO: 316
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

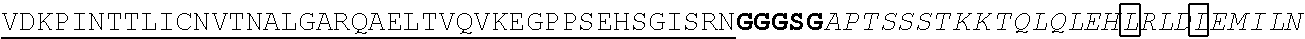

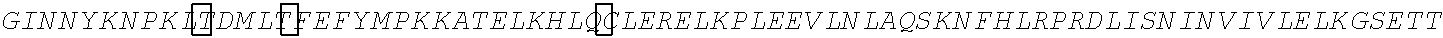

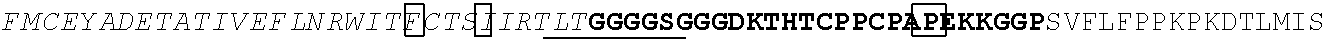

RTPEVTCVVVDVSHEDPEVKFNWYVDGVEVHNAKTKPREEQYNSTYRVVSVLTVLHQDWLNGKEYKCK

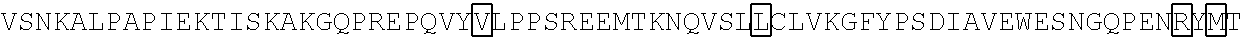

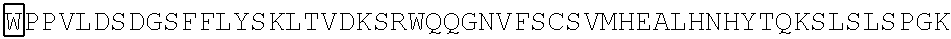

(human PD-L1 extracellular domain mutant2 154Q/E58M/R113T/M115L/S117A/G119K
extracellular domain-linker-single-chain mutant homodimer IFN-Y (A23V/A23V)-linker hinge-IgG1 Fc
mutant1; PD-L1 is underlined; linker is bolded and underlined; hinge is bolded; IFN-y is italicized)
SEQ ID NO: 317

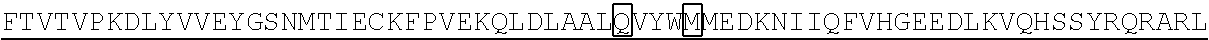

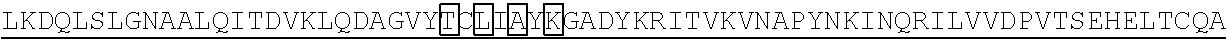

EGYPKAEVIWTSSDHQVLSGKTTTTNSKREEKLFNVTSTLRINTTTNEIFYCTFRRLDPEENHTAELVIPELPL

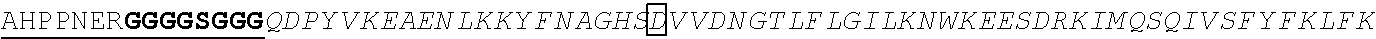

NFKDDOSIQKSVETIKEDMNVKFFNSNKKKRDDFEKLTNYSVTDLNVORKAIHELIQVMAELSPAAKTGKRKRSQ

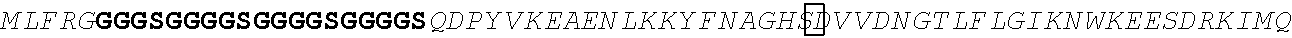

SQIVSFYFKLFKNFKDDOSIQKSVETIKEDMNVKFFNSNKKKRDDFEKLTNYSVTDLNVORKAIHELIQVMAELSP

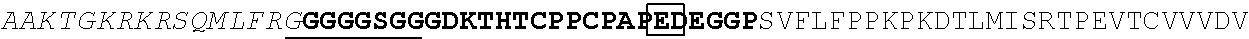

SHEDPEVKFNWYVDGVEVHNAKTKPREEQYNSTYRVVSVLTVLHQDWLNGKEYKCKVSNKALPAPIEKT

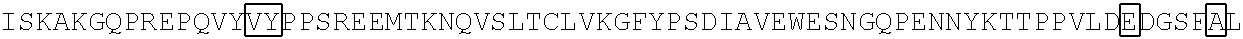

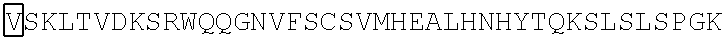

(human PD-L1 extracellular domain mutant7 154Q/E58M/R113T/M115L/S117A
extracellular domain-linker- single-chain mutant homodimer IFN-γ (A23V/A23V)-linker hinge-IgG1 Fc
mutant1; PD-L1 is underlined; linker is bolded and underlined; hinge is bolded; IFN-γ is italicized)
SEQ ID NO: 318

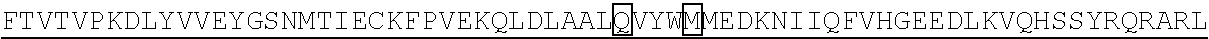

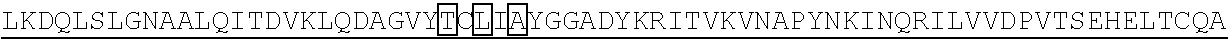

EGYPKAEVIWTSSDHQVLSGKTTTTNSKREEKLFNVTSTLRINTTTNEIFYCTFRRLDPEENHTAELVIPELPL

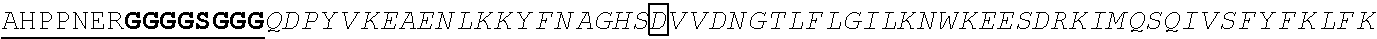

NFKDDOSIQKSVETIKEDMNVKFFNSNKKKRDDFEKLTNYSVTDLNVORKAIHELIQVMAELSPAAKTGKRKRSQ

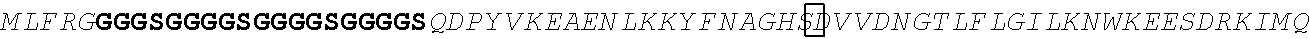

SQIVSFYFKLFKNFKDDQSIQKSVETIKEDMNVKFFNSNKKKRDDFEKLTNYSVTDLNVORKAIHELIQVMAELSP

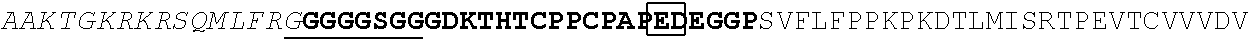

SHEDPEVKFNWYVDGVEVHNAKTKPREEQYNSTYRVVSVLTVLHQDWLNGKEYKCKVSNKALPAPIEKT

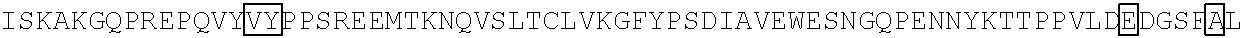

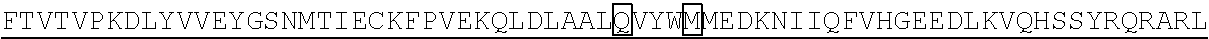

(human PD-L1 extracellular domain mutant2 154Q/E58M/R113T/M115L/S117A/G119K
extracellular domain-linker-single-chain mutant homodimer IFN-γ (A23V/A23V)-linker-hinge-IgG1 Fc
mutant2; PD-L1 is underlined; linker is shaded; hinge is bolded; IFN-γ is italicized
SEQ ID NO: 319

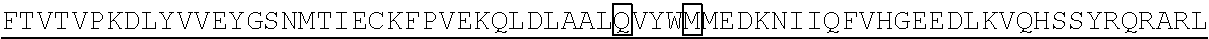

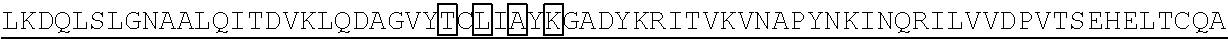

EGYPKAEVIWTSSDHQVLSGKTTTTNSKREEKLFNVTSTLRINTTTNEIFYCTFRRLDPEENHTAELVIPELPL

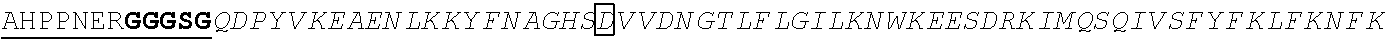

DDQSIQKSVETIKEDMNVKFFNSNKKKRDDFEKLTNYSVTDLNVQRKAIHELIQVMAELSPAAKTGKRKRSQMLF

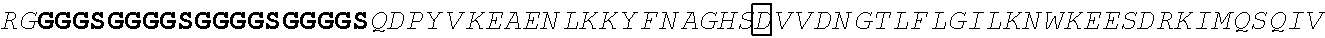

SFYFKLFKNFKDDQSIQKSVETIKEDMNVKFFNSNKKKRDDFEKLTNYSVTDLNVQRKAIHELIQVMAELSPAAKT

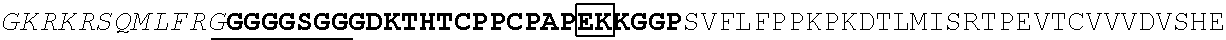

DPEVKFNWYVDGVEVHNAKTKPREEQYNSTYRVVSVLTVLHQDWLNGKEYKCKVSNKALPAPIEKTISK

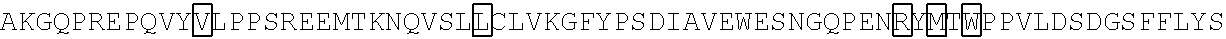

KLTVDKSRWQQGNVFSCSVMHEALHNHYTQKSLSLSPGK
(human PD-L1 extracellular domain mutant7 I54Q/E58M/R113T/M115L/S117A/G119K
extracellular domain-linker- single-chain mutant homodimer IFN-γ (A23V/A23V)-linker -hinge-IgG1 Fc
mutant2; PD-L1 is underlined; linker is bolded and underlined; hinge is bolded; IFN-γ is italicized
SEQ ID NO: 320

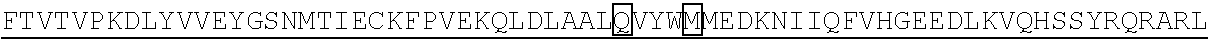

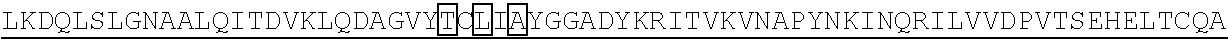

EGYPKAEVIWTSSDHQVLSGKTTTTNSKREEKLFNVTSTLRINTTTNEIFYCTFRRLDPEENHTAELVIPELPL

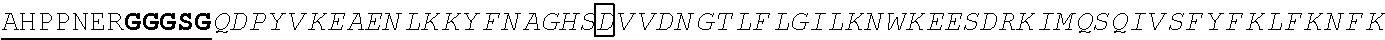

DDQSIQKSVETIKEDMNVKFFNSNKKKRDDFEKLTNYSVTDLNVQRKAIHELIQVMAELSPAAKTGKRKRSQMLF

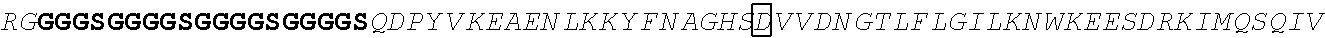

SFYFKLFKNFKDDQSIQKSVETIKEDMNVKFFNSNKKKRDDFEKLTNYSVTDLNVORKAIHELIQVMAELSPAAKT

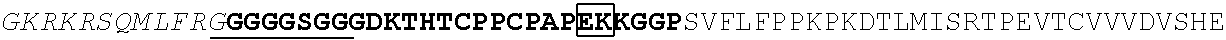

DPEVKFNWYVDGVEVHNAKTKPREEQYNSTYRVVSVLTVLHQDWLNGKEYKCKVSNKALPAPIEKTISK

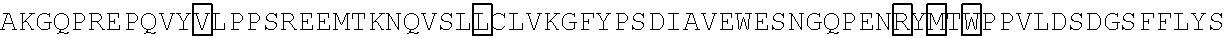

KLTVDKSRWQQGNVFSCSVMHEALHNHYTQKSLSLSPGK
(human PD-L2 extracellular domain mutant2 S58V extracellular domain-linker-single-
chain mutant homodimer IFN-γ (A23V/A23V)-linker-hinge-IgG1 Fc mutant1; PD-L2 is underlined; linker is
bolded and underlined; hinge is bolded; IFN-γ is italicized)
SEQ ID NO: 321

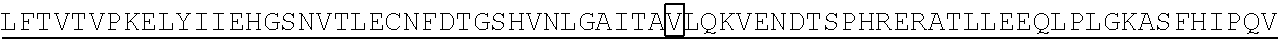

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGQDPYVK

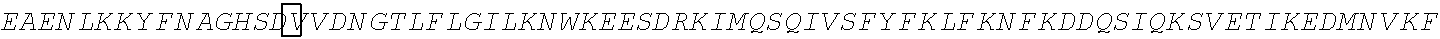

FNSNKKKRDDFEKLTNYSVTDLNVQRKAIHELIQVMAELSPAAKTGKRKRSQMLFRGGGGSGGGGSGGGGSG

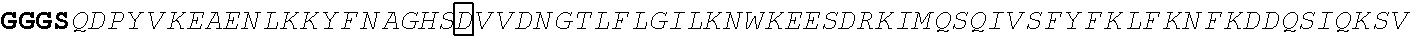

ETIKEDMNVKFFNSNKKKRDDFEKLTNYSVTDLNVORKAIHELIQVMAELSPAAKTGKRKRSQMLFRGGGGGSG

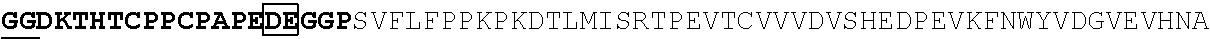

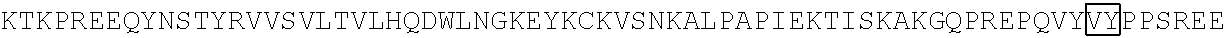

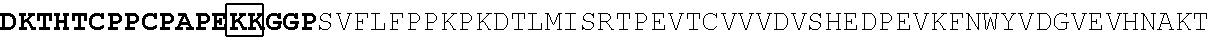

VMHEALHNHYTQKSLSLSPGK
(human PD-L2 extracellular domain mutant4 T56V/S58V/Q60L extracellular domain-
linker- single-chain mutant homodimer IFN-y (A23V/A23V)-linker -hinge-IgG1 Fc mutant1; PD-L2 is
underlined; linker is bolded and underlined; hinge is bolded; IFN-y is italicized)
SEQ ID NO: 322

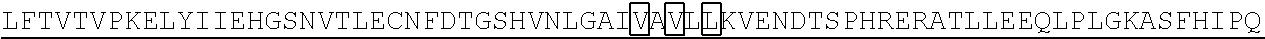

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGQDPYV

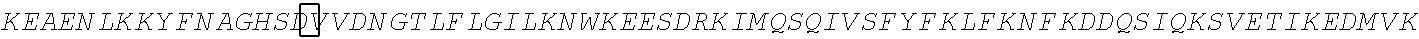

FFNSNKKKRDDFEKLTNYSVTDLNVORKAIHELIQVMAELSPAAKTGKRKRSOMLFRGGGGSGGGGSGGGGS

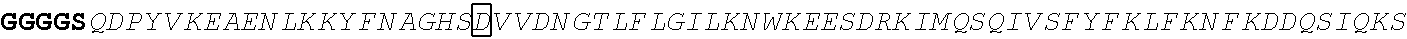

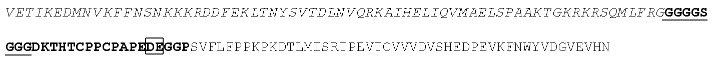

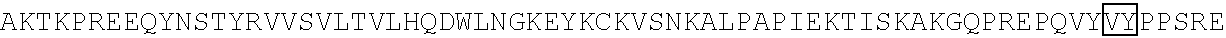

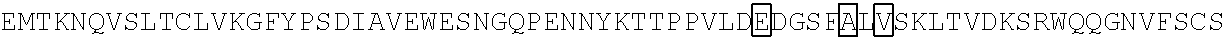

VMHEALHNHYTQKSLSLSPGK
(human PD-L2 extracellular domain mutant2 S58V extracellular domain-linker-single-
chain mutant homodimer IFN-y (A23V/A23V)-linker-hinge-IgG1 Fc mutant2; PD-L2 is underlined; linker is
bolded and underlined; hinge is bolded; IFN-y is italicized
SEQ ID NO: 323

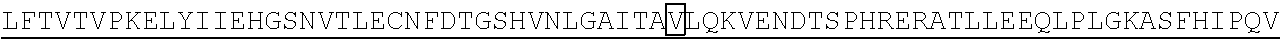

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLOSQMEPRTHPTGGGSGQDPYVKEAEN

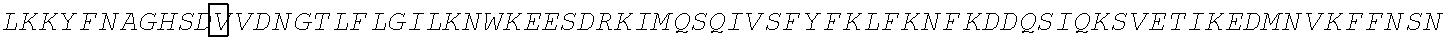

KKKRDDFEKLTNYSVTDLNVQRKAIHELIQVMAELSPAAKTGKRKRSQMLFRGGGGSGGGGSGGGGSGGGG

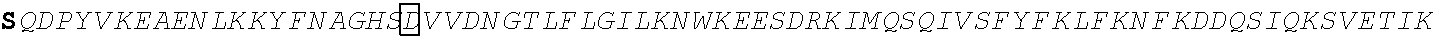

EDMNVKFFNSNKKKRDDFEKLTNYSVTDLNVQRKAIHELIQVMAELSPAAKTGKRKRSQMLFRGGGGGSGGG

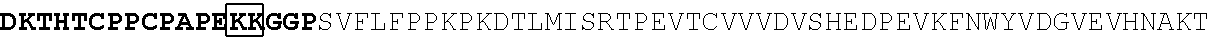

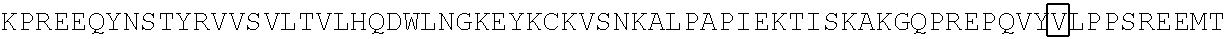

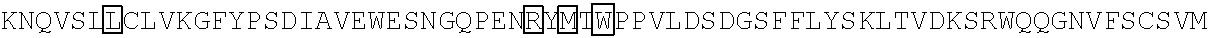

HEALHNHYTQKSLSLSPGK
(human PD-L2 extracellular domain mutant4 T56V/S58V/Q60L extracellular domain-
linker-single-chain mutant homodimer IFN-γ (A23V/A23V)-linker-hinge-IgG1 Fc mutant2; PD-L2 is
underlined; linker is bolded and underlined; hinge is bolded; IFN-γ is italicized
SEQ ID NO: 324

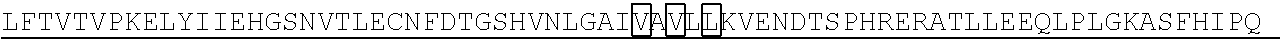

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGQDPYVKEA

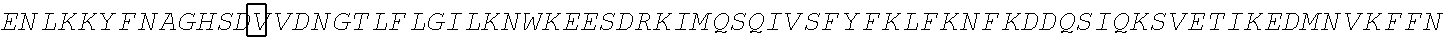

SNKKKRDDFEKLTNYSVTDLNVQRKAIHELIQVMAELSPAAKTGKRKRSQMLFRGGGGSGGGGSGGGGSGGG

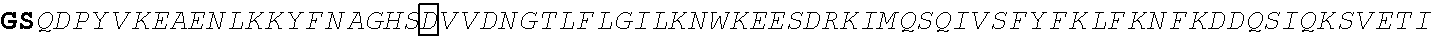

KEDMNVKFFNSNKKKRDDFEKLTNYSVTDLNVQRKAIHELIQVMAELSPAAKTGKRKRSQMLFRGGGGGSGG

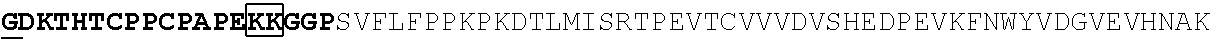

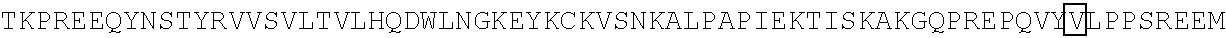

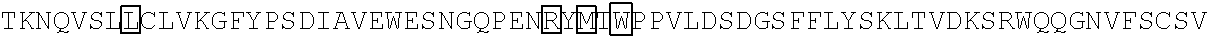

MHEALHNHYTQKSLSLSPGK
(human CD155 extracellular domain-linker-single-chain mutant homodimer IFN-γ
(A23V/A23V)-linker hinge-IgG1 Fc mutant1; CD155 is underlined; linker is bolded and underlined; hinge is
bolded; IFN-γ is italicized)
SEQ ID NO: 325
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVOLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRNGGGGSGGGQDPYVKEAENLKKYFNAGHSD

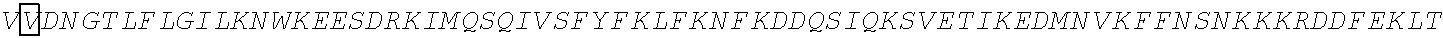

NYSVTDLNVQRKAIHELIQVMAELSPAAKTGKRKRSQMLFRGGGGSGGGGSGGGGSGGGGSQDPYVKEAENL

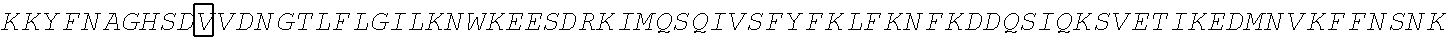

KKRDDFEKLTNYSVTDLNVQRKAIHELIQVMAELSPAAKTGKRKRSQMLFRGGGGGSGGGDKTHTCPPCPAP

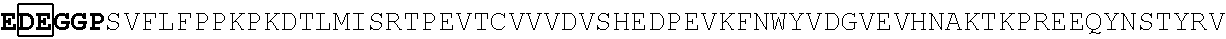

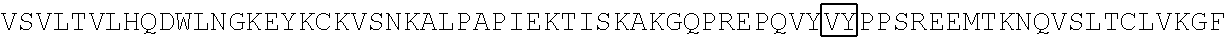

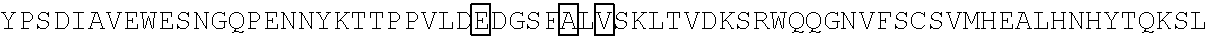

SLSPGK
(human CD155 extracellular domain-linker-single-chain mutant homodimer IFN-γ
(A23V/A23V)-linker-hinge-IgG1 Fc mutant2; CD155 is underlined; linker is bolded and underlined; hinge is
bolded; IFN-γ is italicized
SEQ ID NO: 326
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

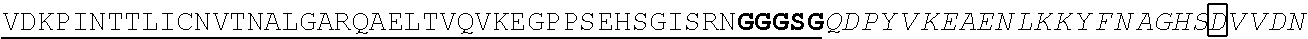

GTLFLGILKNWKEESDRKIMOSQIVSFYFKLFKNFKDDQSIOKSVETIKEDMNVKFFNSNKKKRDDFEKLTNYSVT
DLNVQRKAIHELIQVMAELSPAAKTGKRKRSQMLFRGGGGSGGGGSGGGGSGGGGSQDPYVKEAENLKKYF

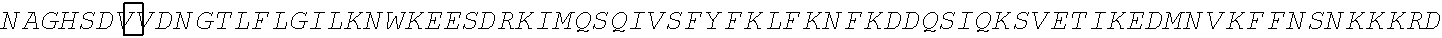

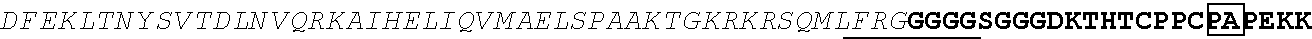

GGPSVFLFPPKPKDTLMISRTPEVTCVVVDVSHEDPEVKFNWYVDGVEVHNAKTKPREEQYNSTYRVVSV

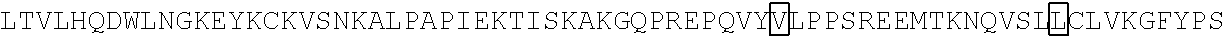

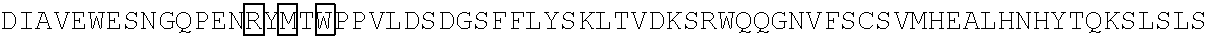

PGK
(human PD-L1 mutant2 154Q/E58M/R113T/M115L/S117A/G119K extracellular domain-
linker-IL-2 mutant R38D/K43E/E61R) linker-hinge-IgG1 Fc mutant2; PD-L1 is underlined; linker is bolded
and underlined; hinge is bolded; IL-12 subunits are italicized
SEQ ID NO: 327

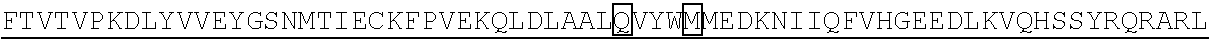

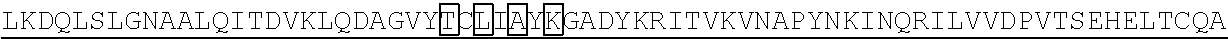

EGYPKAEVIWTSSDHQVLSGKTTTTNSKREEKLFNVTSTLRINTTTNEIFYCTFRRLDPEENHTAELVIPELPL

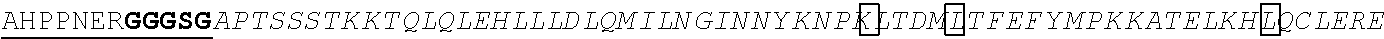

LKPLEEVLNLAOSKNFHLRPRDLISNINVIVLELKGSETTFMCEYADETATIVEFLNRWITFCOSIISTLT

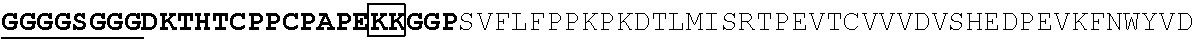

GVEVHNAKTKPREEQYNSTYRVVSVLTVLHQDWLNGKEYKCKVSNKALPAPIEKTISKAKGQPREPQVY

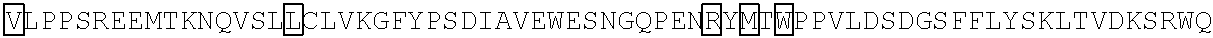

QGNVFSCSVMHEALHNHYTQKSLSLSPGK
(human PD-L1 extracellular domain mutant2 154Q/E58M/R113T/M115L/S117A/G119K
extracellular domain-linker-single-chain IL-12 mutant heterodimer IL-12B (p40 E59A/F60A)-linker IL-12A
(wt p35)-hinge-IgG1 Fc mutant1; PD-L1 is underlined; linker is bolded and underlined; hinge is bolded;
mouse IL-12 subunits are italicized)
SEQ ID NO: 328

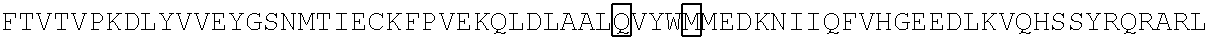

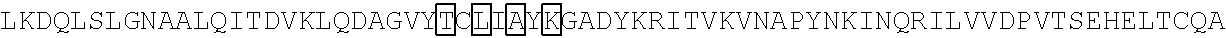

EGYPKAEVIWTSSDHQVLSGKTTTTNSKREEKLFNVTSTLRINTTTNEIFYCTFRRLDPEENHTAELVIPELPL
AHPPNERGGGGSGGGMWELEKDVYVVEVDWTPDAPGETVNLTCDTPEEDDITWTSDORHGVIGSGKTLTITV

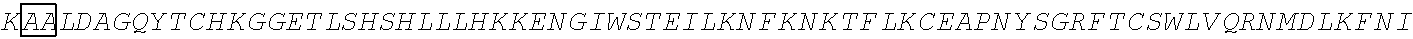

KSSSSSPDSRAVTCGMASLSAEKVTLDORDYEKYSVSCOEDVTCPTAEETLPIELALEAROONKYENYSTSFFIRDII
KPDPPKNLOMKPLKNSOVEVSWEYPDSWSTPHSYFSLKFFVRIORKKEKMKETEEGCNQKGAFLVEKTSTEVQC
KGGNVCVQAQDRYYNSSCSKWACVPCRVRSGGPGGGGSGGGSGGGGSGRVIPVSGPARCLSOSRNLLKTTDD
MVKTAREKLKHYSCTAEDIDHEDITRDOTSTLKTCLPLELHKNESCLATRETSSTTRGSCLPPQKTSLMMTLCLGSI
YEDLKMYQTEFQAINAALQNHNHQQIILDKGMLVAIDELMOSLNHNGETLROKPPVGEADPYRVKMKLCILLHA

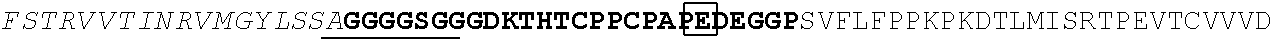

VSHEDPEVKFNWYVDGVEVHNAKTKPREEQYNSTYRVVSVLTVLHQDWLNGKEYKCKVSNKALPAPIEK

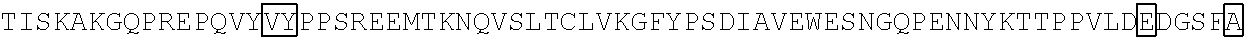

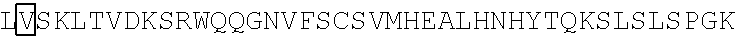

(human PD-L1 extracellular domain mutant2 I54Q/E58M/R113T/M115L/S117A/G119K
extracellular domain-linker-hinge-IgG1 Fc mutant1- linker-single-chain IL-12 mutant heterodimer IL-12B
(p40 E59A/F60A)-linker IL-12A (wt p35); PD-L1 is underlined; linker is bolded and underlined; hinge is
bolded; mouse IL-12 subunits are italicized)
SEQ ID NO: 329

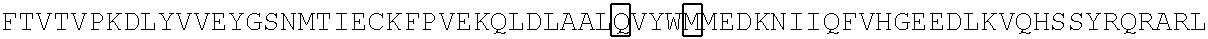

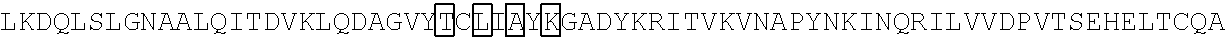

EGYPKAEVIWTSSDHQVLSGKTTTTNSKREEKLFNVTSTLRINTTTNEIFYCTFRRLDPEENHTAELVIPELPL

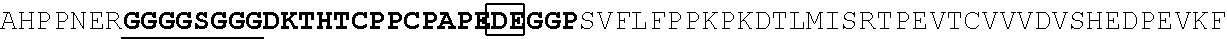

NWYVDGVEVHNAKTKPREEQYNSTYRVVSVLTVLHQDWLNGKEYKCKVSNKALPAPIEKTISKAKGQPR

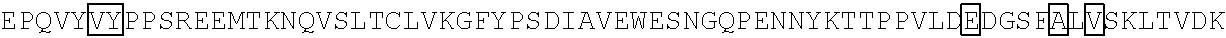

SRWQQGNVFSCSVMHEALHNHYTQKSLSLSPGKGGGGSGGGMWELEKDVYVVEVDWTPDAPGETVNLTC

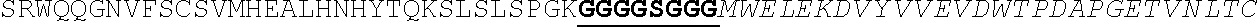

KCEAPNYSGRFTCSWLVQRNMDLKFNIKSSSSSPDSRAVTCGMASLSAEKVTLDQRDYEKYSVSCQEDVTCPTAEE
TLPIELALEARQQNKYENYSTSFFIRDIIKPDPPKNLQMKPLKNSQVEVSWEYPDSWSTPHSYFSLKFFVRIQRKKE
KMKETEEGCNQKGAFLVEKTSTEVQCKGGNVCVQAQDRYYNSSCSKWACVPCRVRSGGPGGGGSGGGSGGG
GSGRVIPVSGPARCLSQSRNLLKTTDDMVKTAREKLKHYSCTAEDIDHEDITRDOTSTLKTCLPLELHKNESCLAT
RETSSTTRGSCLPPQKTSLMMTLCLGSIYEDLKMYQTEFQAINAALQNHNHQQIILDKGMLVAIDELMQSLNHNG
ETLROKPPVGEADPYRVKMKLCILLHAFSTRVVTINRVMGYLSSA
(mouse IL-12 E59A/F60A; linker is bolded and underlined; mouse IL-12 subunits are
italicized)
SEQ ID NO: 330

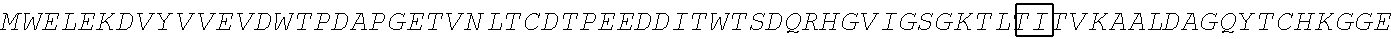

TLSHSHLLLHKKENGIWSTEILKNFKNKTFLKCEAPNYSGRFTCSWLVORNMDLKFNIKSSSSSPDSRAVTCGMAS
LSAEKVTLDORDYEKYSVSCQEDVTCPTAEETLPIELALEARQONKYENYSTSFFIRDIIKPDPPKNLQMKPLKNSQ
VEVSWEYPDSWSTPHSYFSLKFFVRIQRKKEKMKETEEGCNQKGAFLVEKTSTEVQCKGGNVCVQAQDRYYNSS
CSKWACVPCRVRSGGPGGGGSGGGSGGGGSGRVIPVSGPARCLSQSRNLLKTTDDMVKTAREKLKHYSCTAE
DIDHEDITRDQTSTLKTCLPLELHKNESCLATRETSSTTRGSCLPPQKTSLMMTLCLGSIYEDLKMYQTEFQAINAA
LQNHNHQQIILDKGMLVAIDELMOSLNHNGETLRQKPPVGEADPYRVKMKLCILLHAFSTRVVTINRVMGYLSSA
(single-chain IL-12 mutant heterodimer IL-12B (p40 E59A)-linker-IL-12A (wt p35); linker
is bolded and underlined; IL-12 subunits are italicized)
SEQ ID NO: 331

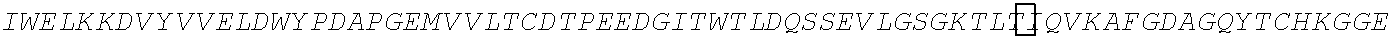

VLSHSLLLLHKKEDGIWSTDILKDOKEPKNKTFLRCEAKNYSGRFTCWWLTTISTDLTFSVKSSRGSSDPQGVTCG
AATLSAERVRGDNKEYEYSVECQEDSACPAAEESLPIEVMVDAVHKLKYENYTSSFFIRDIIKPDPPKNLQLKPLKN
SRQVEVSWEYPDTWSTPHSYFSLTFCVQVQGKSKREKKDRVFTDKTSATVICRKNASISVRAQDRYYSSSWSEWAS
VPCSGGGGSGGGGSGGGGSGGGGSGRNLPVATPDPGMFPCLHHSQNLLRAVSNMLQKARQTLEFYPCTSEE
IDHEDITKDKTSTVEACLPLELTKNESCLNSRETSFITNGSCLASRKTSFMMALCLSSIYEDLKMYQVEFKTMNAKL
LMDPKRQIFLDQNMLAVIDELMQALNFNSETVPQKSSLEEPDFYKTKIKLCILLHAFRIRAVTIDRVMSYLNAS
(single-chain IL-12 mutant heterodimer IL-12B (p40 G64A)-linker-IL-12A (wt p35); linker
is bolded and underlined; IL-12 subunits are italicized)
SEQ ID NO: 332

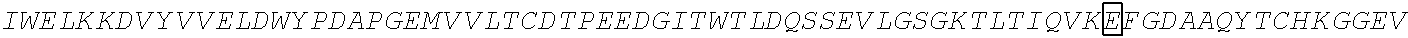

LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECOEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKTSTVEACLPLELTKNESCLNSRETSFITNGSCLASRKTSFMMALCLSSIYEDLKMYQVEFKTMNAKLL
MDPKRQIFLDQNMLAVIDELMQALNFNSETVPQKSSLEEPDFYKTKIKLCILLHAFRIRAVTIDRVMSYLNAS
SEQ ID NO: 333
GGGGSGGGSGGGG
SEQ ID NO: 334
GGGGSGGG
SEQ ID NO: 335
GGPGGGGSGGGSGGGG

1. An immunocytokine comprising: a) an antigen-binding protein specifically recognizing a target antigen; and b) a cytokine or variant thereof, wherein the antigen-binding protein comprises an antigen-binding polypeptide comprising from N′ to C′: an antigen-binding fragment, a hinge region, and an Fc domain subunit or portion thereof, and wherein the cytokine or variant thereof is positioned at the hinge region;
and wherein the target antigen is PD-1:
and the antigen-binding protein specifically binds the target antigen with a KD within a range of 10⁻⁸M<KD≤10⁻⁶M.

2. The immunocytokine of claim 1, wherein in the presence of binding of the antigen-binding protein to the target antigen, the activity of the cytokine or variant thereof increases at least about 20% compared to that in the absence of binding of the antigen-binding protein to the target antigen.

3. The immunocytokine of claim 1, wherein in the absence of binding of the antigen-binding protein to the target antigen, the activity of the cytokine or variant thereof positioned at the hinge region is no more than about 70% of that of a corresponding cytokine or variant thereof in a free state.

4. The immunocytokine of claim 1, wherein the cytokine or variant thereof is a cytokine variant, and wherein the activity of the cytokine variant in a free state is no more than about 80% of that of a corresponding wildtype cytokine in a free state.

5. The immunocytokine of claim 1, wherein the antigen-binding protein comprises two antigen-binding polypeptides each comprising a hinge region, and wherein only one antigen-binding polypeptide comprises the cytokine or variant thereof positioned at the hinge region.

6. The immunocytokine of claim 1, wherein the antigen-binding protein comprises two antigen-binding polypeptides each comprising a hinge region, and wherein each antigen-binding polypeptide comprises a cytokine or variant thereof positioned at the hinge region, and the two cytokines or variants thereof are the same or different.

7. (canceled)

8. The immunocytokine of claim 1, wherein the cytokine or variant thereof is a monomeric or dimeric cytokine or variant thereof, and when the cytokine or variant thereof is dimeric, both subunits of the dimeric cytokine or variant thereof are positioned in tandem at the hinge region of the antigen-binding polypeptide.

9-15. (canceled)

16. The immunocytokine of claim 1, wherein the antigen-binding fragment is L2 or PD-L1.

17. The immunocytokine of claim 1, wherein the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, VEGF, erythropoietin, thrombopoietin, G-CSF, M-CSF, SCF, and GM-CSF.

18. (canceled)

19. The immunocytokine of claim 17, wherein the cytokine or variant thereof is IL-2 or variant thereof.

20. The immunocytokine of claim 19, wherein the IL-2 variant comprises one or more mutations at a position selected from the group consisting of L18, Q22, F24, K35, R38, F42, K43, E61, P65, Q126, and S130 relative to a wildtype IL-2 comprising the sequence of SEQ ID NO: 1.

21-47. (canceled)

48. The immunocytokine of claim 17, wherein the cytokine or variant thereof is IL-12 or variant thereof.

49. The immunocytokine of claim 48, wherein the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit comprising the sequence of SEQ ID NO: 30.

50-51. (canceled)

52. The immunocytokine of claim 49, wherein the IL-12 variant comprises an E59A/F60A mutation or an E59A mutation or an F60A mutation within the p40 subunit relative to a wildtype p40 subunit comprising the sequence of SEQ ID NO: 30.

53. The immunocytokine of claim 52, wherein the p40 subunit of the IL-12 variant comprises the sequence of SEQ ID NO: 31, 32 or 33.

54. The immunocytokine of claim 48, wherein the p40 subunit and the p35 subunit of the IL-12 or variant thereof are connected by a linker.

55. The immunocytokine of claim 54, wherein the IL-12 variant comprises the sequence of SEQ ID NO: 36, 275, 331, or 332.

56-66. (canceled)

67. The immunocytokine of claim 1, wherein the antigen-binding protein is an antibody specifically recognizing PD1, wherein the antigen-binding polypeptide comprising the hinge region is a heavy chain of the antibody, and wherein the cytokine or variant thereof is positioned at the hinge region of the heavy chain.

68-99. (canceled)

100. The immunocytokine of claim 67, wherein the antibody is an anti-PD-1 antibody comprising VH-CDR1 and VH-CDR2, and VH-CDR3 of a VH comprising the sequence of SEQ ID NO: 102, and VL-CDR1, VL-CDR2, and VL-CDR3 of a VL comprising the sequence of SEQ ID NO: 103, and wherein the VH-CDR3 comprises any one of following mutations relative to SEQ ID NO: 102: D100N, D100G, D100R, N99G, N99A, or N99M.

101. The immunocytokine of claim 100, wherein the antibody comprises a light chain comprising the sequence of SEQ ID NO: 106, wherein the cytokine or variant thereof is an IL-12 variant comprising the sequence of SEQ ID NO: 36, and wherein the heavy chain comprising the IL-12 variant positioned at the hinge region comprises the sequence of any one of SEQ ID NOs: 268-273.

102-103. (canceled)

104. The immunocytokine of claim 16, wherein the PD-L2 or PD-L1 comprises the sequence of SEQ ID NO: 176, SEQ ID NO:250, any of SEQ ID NOs: 255-261 or any of SEQ ID NOs: 262-265.

105. The immunocytokine of claim 104,
(i) wherein the cytokine or variant thereof is an IL-2 variant comprising the sequence of SEQ ID NO: 2, and wherein the antigen-binding polypeptide comprising the IL-2 variant positioned at the hinge region comprises the sequence of SEQ ID NO: 180;
(ii) wherein the cytokine or variant thereof is an IFN-α variant comprising the sequence of SEQ ID NO: 4, and wherein the antigen-binding polypeptide comprising the IFN-α variant positioned at the hinge region comprises the sequence of SEQ ID NO: 182;
(iii) wherein the cytokine or variant thereof is an INF-7 variant comprising the sequence of SEQ ID NO: 19, and wherein the antigen-binding polypeptide comprising the IFN-γ variant positioned at the hinge region comprises the sequence of SEQ ID NO: 183;
(iv) wherein the cytokine or variant thereof is an IL-10 variant comprising the sequence of SEQ ID NO: 28, and wherein the antigen-binding polypeptide comprising the IL-10 variant positioned at the hinge region comprises the sequence of SEQ ID NO: 184;
(v) wherein the cytokine or variant thereof is an IL-12 variant comprising the sequence of SEQ ID NO: 36, and wherein the antigen-binding polypeptide comprising the IL-12 variant positioned at the hinge region comprises the sequence of SEQ ID NO: 186;
(vi) wherein the cytokine or variant thereof is an IL-12 variant comprising the sequence of SEQ ID NO: 275, and wherein the antigen-binding polypeptide comprising the IL-12 variant positioned at the hinge region comprises the sequence of SEQ ID NO: 274; or
(vii) wherein the cytokine or variant thereof is an IL-23 variant comprising the sequence of SEQ ID NO: 39, and wherein the antigen-binding polypeptide comprising the IL-23 variant positioned at the hinge region comprises the sequence of SEQ ID NO: 187.

106. The immunocytokine of claim 105, wherein the antigen-binding protein comprises a second antigen-binding polypeptide not comprising the cytokine or variant thereof positioned at the hinge region, and wherein the second antigen-binding polypeptide comprises the sequence of any of SEQ ID NOs: 178, 179, 181, and 185.

107-118. (canceled)

119. An isolated nucleic acid encoding the immunocytokine of claim 1.

120-123. (canceled)

124. A pharmaceutical composition comprising the immunocytokine of claim 1, and optionally a pharmaceutical acceptable carrier.

125. A method of treating or preventing a disease or disorder in an individual, comprising administering to the individual an effective amount of the immunocytokine of claim 1.

126-128. (canceled)

129. The method of claim 125, wherein the disease or disorder is a cancer.

130. The method of claim 129, wherein the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer.

131-134. (canceled)
